(19) United States
(12) Patent Application Publication
(10) Pub. No.: US 2025/0127801 A1
(43) Pub. Date: Apr. 24, 2025

(54) BRIDGED TRICYCLIC CARBAMOYLPYRIDONE COMPOUNDS AND USES THEREOF

(71) Applicant: Gilead Sciences, Inc., Foster City, CA (US)

(72) Inventors: Megan K. Armstrong, San Francisco, CA (US); Chienhung Chou, Dublin, CA (US); Michael O. Clarke, Redwood City, CA (US); Ana Z. Gonzalez Buenrostro, San Mateo, CA (US); Xiaochun Han, San Jose, CA (US); Lan Jiang, Foster City, CA (US); Jiayao Li, Foster City, CA (US); Gregg M. Schwarzwalder, Redwood City, CA (US); Qiaoyin Wu, Foster City, CA (US); Hai Yang, San Mateo, CA (US)

(21) Appl. No.: 18/911,325

(22) Filed: Oct. 10, 2024

Related U.S. Application Data
(60) Provisional application No. 63/543,702, filed on Oct. 11, 2023.

(51) Int. Cl. A61K 31/675; A61K 31/55; A61K 45/06; A61P 31/18; C07D 519/00; C07F 9/6561

(52) U.S. Cl. CPC A61K 31/675; A61K 31/55; A61K 45/06; A61P 31/18; C07D 519/00; C07F 9/6561

(57) ABSTRACT

The present disclosure relates generally to compounds, of Formula I:

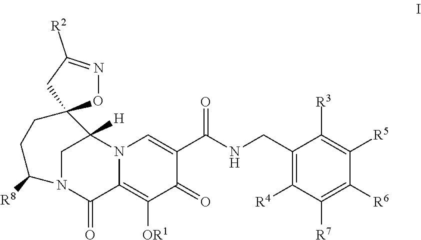

Also disclosed are pharmaceutical compositions comprising said compounds and methods of making said compounds. The compounds of the disclosure are useful in treating or preventing human immunodeficiency virus (HIV) infection.

### CROSS REFERENCE TO RELATED APPLICATIONS

This application claims priority to U.S. Provisional Application No. 63/543,702, filed Oct. 11, 2023, the entire content of which application is hereby incorporated by references in its entirety.

### FIELD

Compounds, compositions, and methods that may be used for treating or preventing human immunodeficiency virus (HIV) infection are disclosed. In particular, novel spirocyclic substituted bridged tricyclic carbamoylpyridone compounds and methods for their preparation and use as therapeutic or prophylactic agents are disclosed.

### BACKGROUND

Human immunodeficiency virus infection and related diseases are a major public health problem worldwide. Human immunodeficiency virus encodes three enzymes which are required for viral replication, reverse transcriptase, protease, and integrase. Although drugs targeting reverse transcriptase and protease are in wide use and have shown effectiveness, particularly when employed in combination, toxicity and development of resistant strains may limit their usefulness (Palella, et al. N. Engl. J Med. (1998) 338:853-860, Richman, D. D. Nature (2001) 410:995-1001). Accordingly, there is a need for new agents that inhibit the replication of HIV.

A goal of antiretroviral therapy is to achieve viral suppression in the HIV infected patient. Current treatment guidelines published by the United States Department of Health and Human Services provide that achievement of viral suppression requires the use of combination therapies, i.e., several drugs from at least two or more drug classes (Panel on Antiretroviral Guidelines for Adults and Adolescents. Guidelines for the Use of Antiretroviral Agents in Adults and Adolescents Living with HIV. Department of Health and Human Services. Available at http://www.aidsinfo.nih.gov/ContentFiles/AdultandAdolescentGL.pdf. Accessed Feb. 12, 2019). In addition, decisions regarding the treatment of HIV infected patients are complicated when the patient requires treatment for other medical conditions (Id. at F-8). Because the standard of care requires the use of multiple different drugs to suppress HIV, as well as to treat other conditions the patient may be experiencing, the potential for drug interaction is a criterion for selection of a drug regimen. As such, there is a need for antiretroviral therapies having a decreased potential for drug interactions.

In addition, the HIV virus is known to mutate in infected subjects (Tang, et al. Drugs (2012) 72 (9) e1-e25). Because of the proclivity of the HIV virus to mutate, there is a need for anti-HIV drugs to be effective against a range of known HIV variants (Hurt, et al. HIV/AIDS CID (2014) 58, 423-431).

For certain patients, for example, those with difficult or limited access to health care, adherence to daily oral treatment or prophylactic regimens can be challenging. Drugs that offer favorable pharmaceutical properties (for example, improved potency, long-acting pharmacokinetics, low clearance, and/or other properties) are amenable to less frequent administration and provide for better patient compliance. Such improvements can, in turn, optimize drug exposure and limit the emergence of drug resistance.

### SUMMARY

The present disclosure is directed to novel compounds having antiviral activity and pharmaceutically acceptable salts thereof. In some embodiments, the compounds may be used to treat HIV infections, to inhibit the activity of HIV integrase and/or to reduce HIV replication. In some embodiments, compounds disclosed herein may be effective against a range of known drug-resistant HIV mutants. In some embodiments, compounds disclosed herein may have a decreased propensity to cause drug-drug interactions when co-administered with other drugs. In some embodiments, compounds disclosed herein may be administered with less than daily frequency, for example, at weekly, monthly, once every three months, once every six months, or longer intervals.

In one embodiment, the disclosure provides a compound of Formula I:

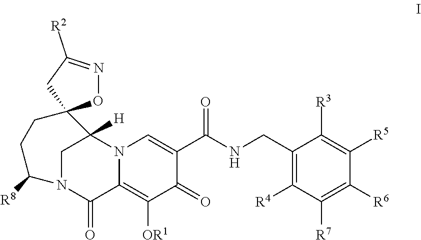

or a pharmaceutically acceptable salt thereof, wherein:
R¹ is —(CR^{1A}R^{1B}O)ₐ(Y)_{b}(CR^{1C}R^{1D})_{d}X;
   wherein a is 0 or 1;
   b is 0 or 1;
   d is 0, 1, 2, or 3;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl;
   Y is —C(O)—, —C(O)O—, —C(O)NH—, —C(O)NR^{1L}, or —P(O)(OH)O—;
      R^{1L} is C₁₋₄alkyl optionally substituted with one or two substituents independently selected from the group consisting of —COOH, —OH, —NH₂ and —CONH₂;
   X is selected from the group consisting of:
      (a) phenyl or pyridyl; wherein the phenyl or pyridyl is optionally substituted with one, two or three substituents independently selected from the group consisting of —OP(O)(OR^{1E})₂, R^{1F}, —COOH, —OCO—C₁₋₂₀alkyl, —CONR^{1X}R^{1Y}, and —NHCO—C₁₋₂₀alkyl,
         wherein each R^{1E} is independently H or phenyl;
         each R^{1F} is independently a C₁₋₃alkyl optionally substituted with one or two substituents independently selected from the group consisting of —COOH, —P(O)(OH)₂, —OP(O)(OR^{1E})₂, —NR^{1G}R^{1H}, —CONR^{1G}R^{1H}, —OCOR^{1I}, and —NHCOR^{1I};
         each R^{1G} is independently H, —COO(CR^{1N}R^{1O})_{c}OP(O)(OH)₂, or C₁₋₄alkyl; wherein the C₁₋₄alkyl is optionally substituted with one, two, or three substituents independently selected from a group consisting of halo, —COOR^{1M}, —OR^{1W}, —P(O)(OH)₂, —NH₂, —NR^{1J}R^{1K}, and —CONR^{1J}R^{1K};
         each R^{1H} is independently H, —COO(CR^{1N}R^{1O})_{c}OP(O)(OH)₂, or C₁₋₄alkyl; wherein the C₁₋₄ alkyl is optionally substituted with one, two, or three substituents independently selected from a group consisting of halo, —COOR^{1M}, —OR^{1W}, —P(O)(OH)₂, —NH₂, —NR^{1J}R^{1K}, and —CONR^{1J}R^{1K}; or
         optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
         each R^{1I} is independently C₁₋₂₀alkyl optionally substituted with one or two substituents selected independently from —P(O)(OH)₂, —COOH and —NR^{1J}R^{1K};
         each R^{1J} is independently H or C₁₋₃alkyl, wherein the C₁₋₃alkyl is optionally substituted with one or two —COOH;
         each R^{1K} is independently H or C₁₋₃alkyl, wherein the C₁₋₃alkyl is optionally substituted with one or two —COOH;
         each R^{1M} is independently H or C₁₋₄alkyl, wherein the C₁₋₄alkyl is optionally substituted with phenyl:
         each R^{1N} is independently H or C₁₋₃alkyl;
         each R^{1O} is independently H or C₁₋₃alkyl;
         each R^{1W} is independently H or C₁₋₃alkyl;
         e is 1, 2, or 3;
         each R^{1X} is H or C₁₋₄alkyl; wherein the C₁₋₄alkyl is optionally substituted with one or two —COOH; and
         each R^{1Y} is H or C₁₋₄alkyl; wherein the C₁₋₄alkyl is optionally substituted with one or two —COOH;
      (b) 4 to 7 membered heterocyclyl comprising 1, 2 or 3 heteroatoms selected from N, O, and S; wherein the heterocyclyl is optionally substituted with one, two or three substituents independently selected from the group consisting of C₁₋₄alkyl, —COOC₁₋₄alkyl, oxo, and —COOCH₂OP(O)(OH)₂, wherein the C₁₋₄alkyl is optionally substituted with —OP(O)(OH)₂ or —NR^{1Z}R^{1AA};
         wherein R^{1Z} is H, —COO(CR^{1AB}R^{1AC}))ₕOP(O)(OH)₂ or C₁₋₄alkyl; wherein the C₁₋₄ alkyl is optionally substituted with one or two —COOH;
         R^{1AB} is H, —COO(CR^{1AD}R^{1AE})ᵢOP(O)(OH)₂ or C₁₋₄alkyl; wherein the C₁₋₄ alkyl is optionally substituted with one or two —COOH;
         each R^{1AB} is independently H or C₁₋₃alkyl,
         each R^{1AC} is independently H or C₁₋₃alkyl;
         each R^{1AD} is independently H or C₁₋₃alkyl;
         each R^{1AE} is independently H or C₁₋₃alkyl;
         h is 1, 2, or 3; and
         i is 1, 2, or 3; and
      (c) C₃₋₇ cycloalkyl optionally substituted with one, two or three substituents independently selected from the group consisting of C₁₋₄alkyl, —COOC₁₋₄alkyl, oxo, —COOR^{1P}, and —OP(O)(OH)₂, wherein the C₁₋₄alkyl is optionally substituted with —OP(O)(OH)₂ or —NR^{1Q}R^{1R};
         wherein R^{1P} is H or C₁₋₃ alkyl,
         R^{1Q} is H, —COO(CR^{1S}R^{1T})ᵢOP(O)(OH)₂, or C₁₋₄alkyl, wherein the C₁₋₄alkyl is optionally substituted with —COOH;
         R^{1R} is H, —COO(CR^{1U}R^{1V})_{g}OP(O)(OH)₂, or C₁₋₄alkyl, wherein the C₁₋₄alkyl is optionally substituted with —COOH;
         each R^{1S}, R^{1T}, R^{1U}, and R^{1V} is independently H or C₁₋₃alkyl;
         f is 1, 2, or 3; and
         g is 1, 2, or 3;
R² is C₁₋₃ alkyl or C₁₋₃ alkoxy;
each R³, R⁴, R³, R⁶ and R⁷ is independently H or halo; and
R⁸ is H or C₁₋₃alkyl.

In one embodiment, the disclosure provides a compound of Formula I, or a pharmaceutically acceptable salt thereof, wherein:
R¹ is —(CR^{1A}R^{1B}O)ₐ(Y)_{b}(CR^{1C}R^{1D})_{d}X;
   wherein a is 0 or 1;
   b is 0 or 1;
   d is 0, 1, 2, or 3;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl;
   Y is —C(O)—, —C(O)O—, —C(O)NH—, —C(O)NR^{1L}—, or —P(O)(OH)O—;
      R^{1L} is C₁₋₄alkyl optionally substituted with one or two substituents independently selected from the group consisting of —COOH, —OH, —NH and —CONH₂;
   X is selected from the group consisting of:
      (a) phenyl or pyridyl; wherein the phenyl or pyridyl is optionally substituted with one, two or three substituents independently selected from the group consisting of —OP(O)(OR^{1E})₂, R^{1F}, —COOH, —OCO—C₁₋₂₀alkyl, and —NHCO—C₁₋₂₀alkyl,
         wherein each R^{1E} is independently H or phenyl;
         each R^{1F} is independently a C₁₋₃alkyl optionally substituted with one or two substituents independently selected from the group consisting of —COOH, —P(O)(OH)₂, —OP(O)(OR^{1E})₂, —NR^{1G}R^{1H}, —CONR^{1G}R^{1H}, —OCOR^{1I}, and —NHCOR^{1I};
         each R^{1G} is independently H, —COO(CR^{1N}R^{1O})ₑOP(O)(OH)₂, or C₁₋₄alkyl; wherein the C₁₋₄ alkyl is optionally substituted with one or two substituents independently selected from a group consisting of —COOR^{1M}, —OH, —NH₂, —NR^{1J}R^{1K}, and —CONR^{1J}R^{1K};
         each R^{1H} is independently H, —COO(CR^{1N}R^{1O})ₑOP(O)(OH)₂, or C₁₋₄alkyl; wherein the C₁₋₄ alkyl is optionally substituted with one or two substituents independently selected from a group consisting of —COOR^{1M}, —OH, —NH₂, —NR^{1J}R^{1K}, and —CONR^{1J}R^{1K}; or
         optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
         each R^{1I} is independently C₁₋₂₀alkyl optionally substituted with one or two substituents selected independently from —COOH and —NR^{1J}R^{1K};
         each R^{1J} is independently H or C₁₋₃alkyl, wherein the C₁₋₃alkyl is optionally substituted with one or two —COOH;
         each R^{1K} is independently H or C₁₋₃alkyl, wherein the C₁₋₃alkyl is optionally substituted with one or two —COOH;
         each R^{1M} is independently H or C₁₋₄alkyl, wherein the C₁₋₄alkyl is optionally substituted with phenyl;
         each R^{1N} is independently H or C₁₋₃alkyl;
         each R^{1O} is independently H or C₁₋₃alkyl;
         e is 1, 2, or 3;
      (b) 4 to 7 membered heterocyclyl comprising 1, 2 or 3 heteroatoms selected from N, O, and S; wherein the heterocyclyl is optionally substituted with one, two or three substituents independently selected from the group consisting of C₁₋₄alkyl, —COOC₁₋₄alkyl, oxo, and —COOCH₂OP(O)(OH)₂, wherein the C₁₋₄alkyl is optionally substituted with —OP(O)(OH)₂; and
      (c) C₃₋₆ cycloalkyl optionally substituted with one, two or three substituents independently selected from the group consisting of C₁₋₄alkyl, —COOC₁₋₄alkyl, oxo, and —OP(O)(OH)₂, wherein the C₁₋₄alkyl is optionally substituted with —OP(O)(OH)₂;
R² is C₁₋₃ alkyl or C₁₋₃ alkoxy;
each R³, R⁴, R³, R⁶ and R⁷ is independently H or halo; and
R⁸ is H or C₁₋₃alkyl.

In one embodiment, a pharmaceutical composition is provided comprising a therapeutically effective amount of a compound of Formula I or a pharmaceutically acceptable salt thereof, and a pharmaceutically acceptable excipient.

In another embodiment, a kit or an article of manufacture is provided comprising a compound of Formula I, or a pharmaceutically acceptable salt thereof, and instructions for use.

In another embodiment, a method of treating an HIV infection in a human having or at risk of having the infection, by administering to the human a therapeutically effective amount of a compound of Formula I or a pharmaceutically acceptable salt thereof, or a pharmaceutical composition of a compound of Formula I, or a pharmaceutically acceptable salt thereof, is provided.

In another embodiment, use of a compound of Formula I, or a pharmaceutically acceptable salt thereof, or a pharmaceutical composition of a compound of Formula I or a pharmaceutically acceptable salt thereof, for treating an HIV infection in a human having or at risk of having the infection is provided.

In another embodiment, use of a compound of Formula I, or a pharmaceutically acceptable salt thereof, or a pharmaceutical composition of a compound of Formula I or a pharmaceutically acceptable salt thereof, in the manufacture of a medicament for treating an HIV infection in a human having or at risk of having the infection is provided.

In another embodiment, a compound of Formula I or a pharmaceutically acceptable salt thereof, or a pharmaceutical composition of a compound of formula I or a pharmaceutically acceptable salt thereof, for use in medical therapy is provided.

In another embodiment, a compound of Formula I or a pharmaceutically acceptable salt thereof, or a pharmaceutical composition of a compound of formula I or a pharmaceutically acceptable salt thereof, for use in treating an HIV infection is provided.

Other embodiments, objects, features, and advantages may be set forth in the detailed description of the embodiments that follows, and in part may be apparent from the description, or may be learned by practice, of the claimed embodiments. These objects and advantages may be realized and attained by the processes and compositions particularly pointed out in the description and claims thereof. The foregoing Summary has been made with the understanding that it is to be considered as a brief and general synopsis of some of the embodiments disclosed herein, is provided for the benefit and convenience of the reader, and is not intended to limit in any manner the scope, or range of equivalents, to which the appended claims are lawfully entitled.

### DETAILED DESCRIPTION

In the following description, certain specific details are set forth in order to provide a thorough understanding of various embodiments disclosed herein. However, one skilled in the art will understand that the embodiments disclosed herein may be practiced without these details. The description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### I. Definitions

Unless the context requires otherwise, throughout the present disclosure and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment disclosed herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Amino” refers to the —NH₂ radical.

“Hydroxy” or “hydroxyl” refers to the —OH radical.

The term “C₁₋ₙ alkyl” as used herein, wherein n is an integer, either alone or in combination with another radical, is intended to mean acyclic, straight or branched chain alkyl radicals containing from 1 to n carbon atoms. “C₁₋₆ alkyl” includes, but is not limited to, methyl, ethyl, propyl (n-propyl), butyl (n-butyl), 1methylethyl (isopropyl), 1methylpropyl (sec-butyl), 2methylpropyl (iso-butyl), 1,1dimethylethyl (tertbutyl), pentyl and hexyl. The abbreviation Me denotes a methyl group; Et denotes an ethyl group, Pr denotes a propyl group, iPr denotes a 1-methylethyl group, Bu denotes a butyl group and tBu denotes a 1,1-dimethylethyl group.

“Alkyl” is hydrocarbon containing normal, secondary or tertiary atoms. For example, an alkyl group can have 1 to 20 carbon atoms (i.e., C₁₋₂₀ alkyl), 1 to 10 carbon atoms (i.e., C₁₋₁₀ alkyl), 1 to 8 carbon atoms (i.e., C₁₋₈ alkyl) or 1 to 6 carbon atoms (i.e., C₁₋₆ alkyl). Examples of suitable alkyl groups include, but are not limited to, methyl (Me, —CH₃), ethyl (Et, CH₂CH₃), 1-propyl (n-Pr, n-propyl, —CH₂CH₂CH₃), 2-propyl (i-Pr, ipropyl, CH(CH₃)₂), 1-butyl (n-Bu, n-butyl, —CH₂CH₂CH₂CH₃), 2methyl1-propyl (i-Bu, ibutyl, —CH₂CH(CH₃)₂), 2butyl (I-Bu, s-butyl, CH(CH₃)CH₂CH₃), 2-methyl-2-propyl (t-Bu, tbutyl, —C(CH₃)₃), 1-pentyl (n-pentyl, CH₂CH₂CH₂CH₂CH₃), 2-pentyl (CH(CH₃)CH₂CH₂CH₃), 3pentyl (CH(CH₂CH₃)₂), 2-methyl-2-butyl (C(CH₃)₂CH₂CH₃), 3methyl2-butyl (CH(CH₃)CH(CH₃)₂), 3methyl1butyl (CH₂CH₂CH(CH₃)₂), 2-methyl-1-butyl (CH₂CH(CH₃)CH₂CH₃), 1-hexyl (CH₂CH₂CH₂CH₂CH₂CH₃), 2-hexyl (CH(CH₃)CH₂CH₂CH₂CH₃), 3hexyl (CH(CH₂CH;)(CH₂CH₂CH₃)), 2methyl-2-pentyl (C(CH₃)₂CH₂CH₂CH₃), 3methyl2pentyl (CH(CH₃)CH(CH₃)CH₂CH₃), 4-methyl-2-pentyl (CH(CH₃)CH₂CH(CH₃)₂), 3-methyl-3-pentyl (—C(CH₃)(CH₂CH₃)₂), 2methyl-3-pentyl (—CH(CH₂CH₃)CH(CH₃)₂), 2,3-dimethyl-2-butyl (C(CH₃)₂CH(CH₃)₂), 3,3dimethyl2butyl (—CH(CH₃)C(CH₃)₃, and octyl ((CH₂)₇CH₃). “Alkyl” also refers to a saturated, branched or straight chain hydrocarbon radical having two monovalent radical centers derived by the removal of two hydrogen atoms from the same or two different carbon atoms of a parent alkane. For example, an alkyl group can have 1 to 10 carbon atoms (i.e., C₁₋₁₀ alkyl), or 1 to 6 carbon atoms (i.e., C₁₋₆ alkyl) or 1-3 carbon atoms (i.e., C₁₋₃ alkyl). Typical alkyl radicals include, but are not limited to, methylene (CH₂), 1,1ethyl (CH(CH₃)), 1,2ethyl (CH₂CH₂), 1,1-propyl (CH(CH₂CH₃)), 1,2-propyl (CH₂CH(CH₃)), 1,3propyl (CH₂CH₂CH₂), 1,4-butyl (CH₂CH₂CH₂CH₂), and the like.

The term “alkenyl” as used herein refers to a straight or branched hydrocarbon containing normal, secondary or tertiary carbon atoms with at least one site of unsaturation, i.e., a carbon-carbon, sp² double bond. For example, an alkenyl group can have 2 to 20 carbon atoms (i.e., C₂-C₂₀ alkenyl, or C₂₋₂₀ alkenyl), 2 to 8 carbon atoms (i.e., C₂-C₈ alkenyl or C₂₋₈), or 2 to 6 carbon atoms (i.e., C₂-C₆ alkenyl, or C₂₋₆ alkenyl). Examples of suitable alkenyl groups include, but are not limited to, ethylene or vinyl (CH═CH₂), allyl (CH₂CH═CH₂), cyclopentenyl (C₅H₇), and 5-hexenyl (CH₂CH₂CH₂CH₂CH═CH₂).

The term “C₂₋ₙalkenyl”, as used herein, wherein n is an integer, either alone or in combination with another radical, is intended to mean an unsaturated, acyclic straight or branched chain radical containing two to n carbon atoms, at least two of which are bonded to each other by a double bond. Examples of such radicals include, but are not limited to, ethenyl (vinyl), 1propenyl, 2propenyl, and 1butenyl. Unless specified otherwise, the term “C₂₋ₙalkenyl” is understood to encompass individual stereoisomers where possible, including but not limited to (E) and (Z) isomers, and mixtures thereof. When a C₂₋ₙalkenyl group is substituted, it is understood to be substituted on any carbon atom thereof which would otherwise bear a hydrogen atom, unless specified otherwise, such that the substitution would give rise to a chemically stable compound, such as are recognized by those skilled in the art.

The term “halo” or “halogen” as used herein refers to fluoro, chloro, bromo and iodo.

The term “heterocyclyl” or “heterocycle” as used herein refers to a single saturated or partially unsaturated ring or a multiple condensed ring. The term includes single saturated or partially unsaturated ring (e.g. 3, 4, 5, 6 or 7-membered ring) from about 1 to 6 carbon atoms and from about 1 to 3 heteroatoms selected from the group consisting of oxygen, nitrogen and sulfur in the ring. The ring may be substituted with one or more (e.g. 1, 2 or 3) oxo groups and the sulfur and nitrogen atoms may also be present in their oxidized forms. Such rings include but are not limited to azetidinyl, tetrahydrofuranyl or piperidinyl. The term also includes multiple condensed ring systems (e.g. ring systems comprising 2 or 3 rings) wherein a heterocycle group (as defined above) can be connected to two adjacent atoms (fused heterocycle) with one or more heterocycles (e.g. decahydronapthyridinyl), heteroaryls (e.g. 1,2,3,4-tetrahydronaphthyridinyl), carbocycles (e.g. decahydroquinolyl) or aryls. It is to be understood that the point of attachment of a heterocycle multiple condensed ring, as defined above, can be at any position of the ring including a heterocyle, heteroaryl, aryl or a carbocycle portion of the ring. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, and 1,3-dioxol-2-onyl.

The term “ring” as used herein refers to cycloalkyl or heterocyclyl group. The term ring as used herein include, but are not limited, to spiro, bridged and fused rings. The ring can be fully saturated or partially unsaturated.

The term “C₃₋ₘ cycloalkyl” as used herein, wherein m is an integer, either alone or in combination with another radical, is intended to mean a cycloalkyl substituent containing from 3 to m carbon atoms and includes, but is not limited to, cyclopropyl, cyclobutyl, cyclopentyl, cyclohexyl and cycloheptyl. The term includes fully saturated as well as partially unsaturated rings.

It is to be understood that when a variable is substituted, for example, as described by the phrase “C₁₋₆ alkyl, either alone or as part of a group, is optionally substituted”, the phrase means that the variable C₁₋₆ alkyl can be substituted when it is alone and that it can also be substituted when the variable “C₁₋₆ alkyl” is part of a larger group. Similarly, when stated, other variables can also be substituted “either alone or as part of a group.”

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers or axes of chirality and whose molecules are not mirror images of one another. Diastereomers typically have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

The term “treatment” or “treating,” to the extent it relates to a disease or condition includes preventing the disease or condition from occurring, inhibiting or ameliorating the disease or condition (e.g., arresting or slowing its development), eliminating the disease or condition (e.g., causing regression or cure of the disease or condition), and/or relieving one or more symptoms of the disease or condition. In the case of HIV, treatment includes reducing the level of HIV viral load in a patient.

In some embodiments, the term “treatment” refers to the administration of a compound or composition according to the present invention to alleviate or eliminate symptoms of HIV infection and/or to reduce viral load in a patient. The term “treatment” also encompasses the administration of a compound or composition according to the present invention before the exposure of the individual to the virus, postexposure of the individual to the virus but before the appearance of symptoms of the disease, and/or prior to the detection of the virus in the blood, to prevent the appearance of symptoms of the disease and/or to prevent the virus from reaching detectible levels in the blood, and the administration of a compound or composition according to the present invention to prevent perinatal transmission of HIV from mother to baby, by administration to the mother before giving birth and to the child within the first days of life.

“Protecting group” refers to a moiety of a compound that masks or alters the properties of a functional group or the properties of the compound as a whole. Chemical protecting groups and strategies for protection/deprotection are well known in the art. See e.g., Protective Groups in Organic Chemistry, Theodora W. Greene, John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection. e.g., alcohols, be physiologically acceptable, although in general it is more desirable if the products are pharmacologically innocuous.

Protecting groups are available, commonly known and used, and are optionally used to prevent side reactions with the protected group during synthetic procedures. i.e., routes or methods to prepare the compounds of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis. PGs do not need to be, and generally are not, the same if the compound is substituted with multiple PG. In general, PG will be used to protect functional groups such as carboxyl, hydroxyl, thio, or amino groups and to thus prevent side reactions or to otherwise facilitate the synthetic efficiency. The order of deprotection to yield free deprotected groups is dependent upon the intended direction of the synthesis and the reaction conditions to be encountered, and may occur in any order as determined by the artisan.

Various functional groups of the compounds of the invention may be protected. For example, protecting groups for —OH groups (whether hydroxyl, carboxylic acid, phosphonic acid, or other functions) include “ether- or ester-forming groups”. Ether- or ester-forming groups are capable of functioning as chemical protecting groups in the synthetic schemes set forth herein. However, some hydroxyl and thio protecting groups are neither ether- nor ester-forming groups, as will be understood by those skilled in the art, and are included with amides, discussed below.

A very large number of hydroxyl protecting groups and amide-forming groups and corresponding chemical cleavage reactions are described in Protective Groups in Organic Synthesis, Theodora W. Greene (John Wiley & Sons, Inc., New York, 1991, ISBN 0-471-62301-6) (“Greene”). See also Kocienski, Philip J.; Protecting Groups (Georg Thieme Verlag Stuttgart, New York, 1994), which is incorporated by reference in its entirety herein. In particular Chapter 1, Protecting Groups: An Overview, pages 1-20, Chapter 2, Hydroxyl Protecting Groups, pages 21-94, Chapter 3, Diol Protecting Groups, pages 95-117, Chapter 4, Carboxyl Protecting Groups, pages 118-154, Chapter 5, Carbonyl Protecting Groups, pages 155-184. For protecting groups for carboxylic acid, phosphonic acid, phosphonate, sulfonic acid and other protecting groups for acids see Greene as set forth below.

The term “solvate” refers to a crystalline solid containing amounts of a solvent incorporated within the crystal structure. As used herein, the term “solvate” includes hydrates.

The term “non-solvate” refers to a crystalline solid in which no solvent molecules occupy a specific crystallographic site.

The term “pharmaceutically acceptable” with respect to a substance as used herein means that substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

The term “pharmaceutically acceptable salt” as used herein is intended to mean a salt of a compound according to the invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. Lists of suitable salts are found in, for example, S. M. Birge et al., J. Pharm. Sci., 1977, 6, pp. 1-19.

The term “pharmaceutically-acceptable acid addition salt” as used herein is intended to mean those salts which retain the biological effectiveness and properties of the free bases and which are not biologically or otherwise undesirable, formed with inorganic acids including but not limited to hydrochloric acid, hydrobromic acid, sulfuric acid, sulfamic acid, nitric acid, phosphoric acid and the like, and organic acids including but not limited to acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutanic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid and the like.

The term “pharmaceutically-acceptable base addition salt” as used herein is intended to mean those salts which retain the biological effectiveness and properties of the free acids and which are not biologically or otherwise undesirable, formed with inorganic bases including but not limited to ammonia or the hydroxide, carbonate, or bicarbonate of ammonium or a metal cation such as sodium, potassium, lithium, calcium, magnesium, iron, zinc, copper, manganese, aluminum and the like. Particularly preferred are the ammonium, potassium, sodium, calcium, and magnesium salts. Salts derived from pharmaceutically-acceptable organic nontoxic bases include but are not limited to salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine. N-methylmorpholine, dicyclohexylamine, dibenzylamine. N,N-dibenzylphenethylamine, 1-ephenamine, N,N′-dibenzylethylenediamine, polyamine resins and the like. Particularly preferred organic nontoxic bases are isopropylamine, diethylamine, ethanolamine, trimethylamine, dicyclohexylamine, choline, and caffeine.

The term “antiviral agent” as used herein is intended to mean an agent that is effective to inhibit the formation and/or replication of a virus in a mammal, including but not limited to agents that interfere with either host or viral mechanisms necessary for the formation and/or replication of a virus in a mammal. The term “antiviral agent” includes, for example, an HIV integrase catalytic site inhibitor selected from the group consisting: raltegravir (ISENTRESS®; Merck); elvitegravir (Gilead); soltegravir (GSK; ViiV); GSK 1265744 (GSK; ViiV) and dolutegravir; an HIV nucleoside reverse transcriptase inhibitor selected from the group consisting of: abacavir (ZIAGEN®; GSK); didanosine (VIDEX®; BMS); tenofovir (VIREAD®; Gilead); emtricitabine (EMTRIVA®; Gilead); lamivudine (EPIVIR®; GSK/Shire); stavudine (ZERIT®; BMS); zidovudine (RETROVIR®; GSK); elvucitabine (Achillion); and festinavir (Oncolys); an HIV non-nucleoside reverse transcriptase inhibitor selected from the group consisting of: nevirapine (VIRAMUNE®; BI); efavirenz (SUSTIVA®; BMS); etravirine (INTELENCE®; J&J); rilpivirine (TMC278, R278474; J&J); fosdevirine (GSK/ViiV); and lersivirine (Pfizer ViiV); an HIV protease inhibitor selected from the group consisting of: atazanavir (REYATAZ®; BMS); darunavir (PREZISTA®; J&J); indinavir (CRIXIVAN®; Merck); lopinavir (KELETRA®; Abbott); nelfinavir (VIRACEPT®; Pfizer); saquinavir (INVIRASE®; Hoffmann-LaRoche); tipranavir (APTIVUS®; BI); ritonavir (NORVIR®; Abbott); and fosamprenavir (LEXIVA); GSK/Vertex); an HIV entry inhibitor selected from: maraviroc (SELZENTRY®; Pfizer); enfuvirtide (FUZEON®; Trimeris); and BMS-663068 (BMS); and an HIV maturation inhibitor selected from: bevirimat (Myriad Genetics).

The term “inhibitor of HIV replication” as used herein is intended to mean an agent capable of reducing or eliminating the ability of HIV to replicate in a host cell, whether in vitro, ex vivo or in vivo.

The term “substituent”, as used herein and unless specified otherwise, is intended to mean an atom, radical or group which may be bonded to a carbon atom, a heteroatom or any other atom which may form part of a molecule or fragment thereof, which would otherwise be bonded to at least one hydrogen atom. Substituents contemplated in the context of a specific molecule or fragment thereof are those which give rise to chemically stable compounds, such as are recognized by those skilled in the art.

The term “heteroatom” as used herein is intended to mean 0, S or N.

The terms “O—C₁₋ₙ alkyl” or “C₁₋ₙ alkoxy” as used herein interchangeably, wherein n is an integer, either alone or in combination with another radical, is intended to mean an oxygen atom further bonded to an alkyl radical having 1 to n carbon atoms as defined above. Examples of C₁₋ₙ alkoxy include but are not limited to methoxy (CH₃O—), ethoxy (CH₃CH₂O—), propoxy (CH₃CH₂CH₂O—), 1-methylethoxy (iso-propoxy; (CH₃)₂CHO) and 1,1-dimethylethoxy (tert-butoxy; (CH₃)₃CO). When an C₁₋ₙ alkoxy is substituted, it is understood to be substituted on the alkyl portion thereof, such that the substitution would give rise to a chemically stable compound, such as are recognized by those skilled in the art.

The term “mammal” as used herein is intended to encompass humans, as well as non-human mammals which are susceptible to infection by HIV. Non-human mammals include but are not limited to domestic animals, such as cows, pigs, horses, dogs, cats, rabbits, rats and mice, and non-domestic animals.

The embodiments disclosed herein are also meant to encompass all pharmaceutically acceptable compounds of Formula I being isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷I, ¹⁸O, ³¹P, ³²P, ³⁵S, ¹⁸F, ³⁶Cl, ¹²¹I, and ¹²⁵I, respectively. In certain embodiments, these radiolabeled compounds are useful to help determine or measure the effectiveness of the compounds, by characterizing, for example, the site or mode of action, or binding affinity to pharmacologically important site of action. Certain isotopically-labeled compounds of Formula I, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

In certain embodiments, substitution with heavier isotopes such as deuterium. i.e., ²H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as ¹¹C, ¹⁸F, ¹⁵O, and ¹³N, can be useful in Positron Emission Topography (PET) studies for examining substrate receptor occupancy. Isotopically-labeled compounds of the compounds disclosed herein can be prepared by techniques known to those skilled in the art or by processes analogous to those described in the Examples as set out below using an appropriate isotopically-labeled reagent in place of the non-labeled reagent previously employed.

The methods, compositions, kits and articles of manufacture provided herein use or include compounds (e.g., a compound of Formula I) or pharmaceutically acceptable salts thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds increase resistance to metabolism, and thus are useful for increasing the half-life of compounds or pharmaceutically acceptable salts thereof, when administered to a mammal. See, e.g., Foster, “Deuterium Isotope Effects in Studies of Drug Metabolism”. Trends Pharmacol. Sci., 5(12):524-527 (1984). Such compounds can be synthesized by means known in the art, for example by employing starting materials in which one or more hydrogen atoms have been replaced by deuterium.

The embodiments disclosed herein are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the embodiments disclosed herein include compounds produced by a process comprising administering a compound according to the embodiments disclosed herein to a mammal for a period of time sufficient to yield a metabolic product thereof. Such products are typically identified by administering a radiolabeled compound according to the embodiments disclosed herein in a detectable dose to an animal, such as rat, mouse, guinea pig, monkey, or to human, allowing sufficient time for metabolism to occur, and isolating its conversion products from the urine, blood or other biological samples.

The compounds disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic, scalemic, and optically pure forms. Optically active (+) and (−), (R)- and (S)-, or (D)- and (L)-isomers may be prepared using chiral synthons or chiral reagents, or resolved using methods such as chromatography and fractional crystallization. Techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Optional” or “optionally” means that the subsequently described event or circumstances may or may not occur, and that the description includes instances where said event or circumstance occurs and instances in which it does not. For example. “optionally substituted heterocyclyl” means that the heterocyclyl radical may or may not be substituted and that the description includes both substituted heterocyclyl radicals and heterocyclyl radicals having no substitution.

### II. Compounds

In some embodiments, the disclosure provides a compound of Formula I:

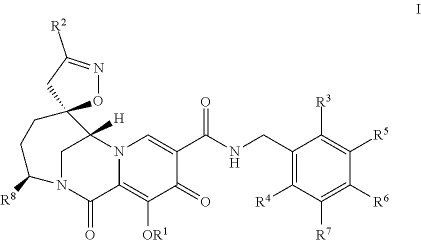

or a pharmaceutically acceptable salt thereof, wherein:
R¹ is (CR^{1A}R^{1B}O)ₐ(Y)_{b}(CR^{1C}R^{1D})_{d}X;
   wherein a is 0 or 1;
   b is 0 or 1;
   d is 0, 1, 2, or 3
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl;
   Y is —C(O)—, —C(O)O—, —C(O)NH—, —C(O)NR^{1L}, or —P(O)(OH)O—;
      R^{1L} is C₁₋₄alkyl optionally substituted with one or two substituents independently selected from the group consisting of —COOH, —OH, —NH₂ and —CONH₂;
   X is selected from the group consisting of:
      (a) phenyl or pyridyl; wherein the phenyl or pyridyl is optionally substituted with one, two or three substituents independently selected from the group consisting of —OP(O)(OR^{1E})₂, R^{1F}, —COOH, —OCO—C₁₋₂₀alkyl, —CONR^{1X}R^{1Y}, and —NHCO—C₁₋₂₀alkyl,
         wherein each RE is independently H or phenyl;
         each R^{1F} is independently a Ct-3alkyl optionally substituted with one or two substituents independently selected from the group consisting of —COOH, —P(O)(OH)₂, —OP(O)(OR^{1E})₂, —NR^{1G}R^{1H}, —CONR^{1G}R^{1H}, —OCOR^{1I}, and —NHCOR^{1I};
         each R^{1G} is independently H, —COO(CR^{1N}NR^{1O})ₑOP(O)(OH)₂, or C₁₋₄alkyl; wherein the C₁₋₄ alkyl is optionally substituted with one, two, or three substituents independently selected from a group consisting of halo, —COOR^{1M}, —OR^{1W}, —P(O)(OH)₂, —NH₂, —NR^{1J}R^{1K}, and —CONR^{1J}R^{1K};
         each R^{1H} is independently H, —COO(CR^{1N}NR^{1O})ₑOP(O)(OH)₂, or C₁₋₄alkyl; wherein the C₁₋₄ alkyl is optionally substituted with one, two, or three substituents independently selected from a group consisting of halo, —COOR^{1M}, —OR^{1W}, —P(O)(OH)₂, —NH₂, —NR^{1J}R^{1K}, and —CONR^{1J}R^{1K}; or
         optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
         each R^{1I} is independently C₁₋₂₀alkyl optionally substituted with one or two substituents selected independently from —P(O)(OH)₂, —COOH and —NR^{1J}R^{1K}
         each R^{1J} is independently H or C₁₋₃alkyl, wherein the C₁₋₃alkyl is optionally substituted with one or two —COOH;
         each R^{1K} is independently H or C₁₋₃alkyl, wherein the C₁₋₃alkyl is optionally substituted with one or two —COOH;
         each R^{1M} is independently H or C₁₋₄alkyl, wherein the C₁₋₄alkyl is optionally substituted with phenyl;
         each R^{1N} is independently H or C₁₋₃alkyl;
         each R^{1O} is independently H or C₁₋₃alkyl;
         each R^{1W} is independently H or C₁₋₃alkyl;
         e is 1, 2, or 3;
         each R^{1X} is H or C₁₋₄alkyl; wherein the C₁₋₄alkyl is optionally substituted with one or two —COOH; and
         each R^{1Y} is H or C₁₋₄alkyl; wherein the C₁₋₄alkyl is optionally substituted with one or two —COOH;
      (b) 4 to 7 membered heterocyclyl comprising 1, 2 or 3 heteroatoms selected from N, O, and S; wherein the heterocyclyl is optionally substituted with one, two or three substituents independently selected from the group consisting of C₁₋₄alkyl, —COOC₁₋₄alkyl, oxo, and —COOCH₂OP(O)(OH)₂, wherein the C₁₋₄alkyl is optionally substituted with —OP(O)(OH)₂ or —NR^{1Z}R^{1AA};
         wherein R^{1Z} is H, —COO(CR^{1AB}R^{1AC})ₕOP(O)(OH)₂ or C₁₋₄alkyl; wherein the C₁₋₄ alkyl is optionally substituted with one or two —COOH;
         R^{1AA} is H, —COO(CR^{1AD}R^{1AE})ᵢOP(O)(OH)₂ or C₁₋₄alkyl; wherein the C₁₋₄ alkyl is optionally substituted with one or two —COOH;
         each R^{1AB} is independently H or C₁₋₃alkyl;
         each R^{1AC} is independently H or C₁₋₃alkyl;
         each R^{1AD} is independently H or C₁₋₃alkyl,
         each R^{1AE} is independently H or C₁₋₃all;
         h is 1, 2, or 3; and
         i is 1, 2, or 3; and
      (c) C₃₋₇ cycloalkyl optionally substituted with one, two or three substituents independently selected from the group consisting of C₄alkyl, —COOC₁₋₄alkyl, oxo, —COOR^{1P}, and —OP(O)(OH)₂, wherein the C₁₋₄alkyl is optionally substituted with —OP(O)(OH)₂ or —NR^{1Q}R^{1R},
         wherein R^{1P} is H or C₁₋₃ alkyl;
         R^{1Q} is H, —COO(CR^{1S}R^{1T})_{f}OP(O)(OH)₂, or C₁₋₄alkyl, wherein the C₁₋₄alkyl is optionally substituted with —COOH;
         R^{1R} is H, —COO(CR^{1U}R^{1V})_{g}OP(O)(OH)₂, or C₁₋₄alkyl, wherein the C₁₋₄alkyl is optionally substituted with —COOH;
         each R^{1S}, R^{1T}, R^{1U}, and R^{1V} is independently H or C₁₋₃alkyl;
         f is 1, 2, or 3; and
         g is 1, 2, or 3;
R² is C₁₋₃ alkyl or C₁₋₃ alkoxy;
each R³, R⁴, R⁵, R⁶ and R⁷ is independently H or halo; and
   R⁸ is H or C₁₋₃alkyl.

In some embodiments, the disclosure provides a compound of Formula I, or a pharmaceutically acceptable salt thereof, wherein:
R¹ is —(CR^{1A}R^{1B}O)ₐ(Y)_{b}(CR^{1C}R^{1D})_{d}X;
   wherein a is 0 or 1;
   b is 0 or 1;
   d is 0, 1, 2, or 3;
   R^{1A} is H or C₁₋₃alkyl;
   R^{1B} is H or C₁₋₃alkyl,
   each R^{1C} is independently H or C₁₋₃alkyl;
   each R^{1D} is independently H or C₁₋₃alkyl;
   Y is —C(O)—, —C(O)O—, —C(O)NH—, —C(O)NR^{1L}, or —P(O)(OH)O—;
      R^{1L} is C₁₋₄alkyl optionally substituted with one or two substituents independently selected from the group consisting of —COOH, —OH, —NH₂ and —CONH₂;
   X is selected from the group consisting of:
      (a) phenyl or pyridyl; wherein the phenyl or pyridyl is optionally substituted with one, two or three substituents independently selected from the group consisting of —OP(O)(OR^{1E})₂, R^{1F}, —COOH, —OCO—C₁₋₂₀alkyl, and —NHCO—C₁₋₂₀alkyl,
         wherein each R^{1E} is independently H or phenyl;
         each R^{1F} is independently a C₁₋₃alkyl optionally substituted with one or two substituents independently selected from the group consisting of —COOH, —P(O)(OH)₂, —OP(O)(OR^{1E})₂, —NR^{1G}R^{1H}, —CONR^{1G}R^{1H}, —OCOR^{1I}, and —NHCOR^{1I};
         each R^{1G} is independently H, —COO(CR^{1N}R^{1O})ₑOP(O)(OH)₂, or C₁₋₄alkyl; wherein the C₁₋₄ alkyl is optionally substituted with one or two substituents independently selected from a group consisting of —COOR^{1M}, —OH, —NH₂, —NR^{1J}R^{1K}, and —CONR^{1J}R^{1K};
         each R^{1H} is independently H, —COO(CR^{1N}R^{1O})ₑOP(O)(OH)₂, or C₁₋₄alkyl; wherein the C₁₋₄ alkyl is optionally substituted with one or two substituents independently selected from a group consisting of —COOR^{1M}, —OH, —NH₂, —NR^{1J}R^{1K}, and —CONR^{1J}R^{1K}; or
         optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
         each R^{1I} is independently C₁₋₂₀alkyl optionally substituted with one or two substituents selected independently from —COOH and —NR^{1J}R^{1K};
         each R^{1J} is independently H or C₁₋₃alkyl, wherein the C₁₋₃alkyl is optionally substituted with one or two —COOH;
         each R^{1K} is independently H or C₁₋₃alkyl, wherein the C₁₋₃alkyl is optionally substituted with one or two —COOH;
         each R^{1N} is independently H or C₁₋₄alkyl, wherein the C₁₋₄alkyl is optionally substituted with phenyl;
         each R^{1N} is independently H or C₁₋₃alkyl;
         each R^{1O} is independently H or C₁₋₃alkyl;
         e is 1, 2, or 3;
      (b) 4 to 7 membered heterocyclyl comprising 1, 2 or 3 heteroatoms selected from N, O, and S; wherein the heterocyclyl is optionally substituted with one, two or three substituents independently selected from the group consisting of C₁₋₄alkyl, —COOC₁₋₄alkyl, oxo, and —COOCH₂OP(O)(OH)₂, wherein the C₁₋₄alkyl is optionally substituted with —OP(O)(OH)₂; and
      (c) C₃₋₆ cycloalkyl optionally substituted with one, two or three substituents independently selected from the group consisting of C₁₋₄alkyl, —COOC₁₋₄alkyl, oxo, and —OP(O)(OH)₂, wherein the C₁₋₄alkyl is optionally substituted with —OP(O)(OH)₂;
R² is C₁₋₃ alkyl or C₁₋₃ alkoxy;
each R³, R⁴, R⁵, R⁶ and R⁷ is independently H or halo, and
R⁸ is H or C₁₋₃alkyl.

In some embodiments, a is 0. In some embodiments, a is 1.

In some embodiments, b is 0. In some embodiments, b is 1.

In some embodiments, d is 0. In some embodiments, d is 1. In some embodiments, d is 2. In some embodiments, d is 3.

In some embodiments, each R^{1A} is H. In some embodiments, each R^{1B} is H. In some embodiments, each R^{1A} and R^{1B} is H.

In some embodiments, each R^{1C} is H. In some embodiments, each R^{1D} is H. In some embodiments, each R^{1C} and R^{1D} is H. In some embodiments, each R^{1C} is independently H or methyl. In some embodiments, each R^{1D} is independently H or methyl.

In some embodiments, Y is —C(O)—, —C(O)O—, —C(O)NH—, or —C(O)NR¹. In some embodiments, R^{1L} is —CH₃. In some embodiments, Y is —C(O)—, —C(O)O—, —C(O)NH—, —C(O)NCH₃—, or —P(O)(OH)O—. In some embodiments, Y is —C(O)—, —C(O)O— or —C(O)NCH₃—. In some embodiments, Y is —C(O)—. In some embodiments, Y is —C(O)O—. In some embodiments, Y is —C(O)NR^{1L}—. In some embodiments, Y is —C(O)NCH₃—. In some embodiments, Y is P(O)(OH)O—.

In some embodiments, X is phenyl or pyridyl; wherein the phenyl or pyridyl is optionally substituted with one, two or three substituents independently selected from the group consisting of —OP(O)(OR^{1F})₂, R^{1F}, —COOH, —OCO—C₁₋₂₀alkyl, —CONR^{1X}R^{1Y}, and —NHCO—C₁₋₂₀alkyl;
wherein each R^{1E} is independently H or phenyl;
each R^{1F} is independently a C₁₋₃alkyl optionally substituted with one or two substituents independently selected from the group consisting of —COOH, —P(O)(OH)₂, —OP(O)(OR^{1E})₂, —NR^{1G}R^{1H}, —CONR^{1G}R^{1H}, —OCOR^{1I}, and —NHCOR^{1I};
   each R^{1G} is independently H, —COO(CR^{1N}R^{1O})ₑOP(O)(OH)₂, or C₁₋₄alkyl; wherein the C₁₋₄ alkyl is optionally substituted with one, two, or three substituents independently selected from a group consisting of halo, —COOR^{1M}, —OR^{1W}, —P(O)(OH)₂, —NH₂, —NR^{1J}R^{1K}, and —CONR^{1J}R^{1K};
   each R^{1H} is independently H, —COO(CR^{1N}R^{1O})ₑOP(O)(OH)₂, or C₁₋₄alkyl; wherein the C₁₋₄ alkyl is optionally substituted with one, two, or three substituents independently selected from a group consisting of halo, —COOR^{1M}, —OR^{1W}, —P(O)(OH)₂, —NH₂, —NR^{1J}R^{1K}, and —CONR^{1J}R^{1K}; or
   optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
   each R^{1I} is independently C₁₋₂₀alkyl optionally substituted with one or two substituents selected independently from —P(O)(OH)₂, —COOH and —NR^{1J}R^{1K};
      each R^{1J} is independently H or C₁₋₃alkyl, wherein the C₁₋₃alkyl is optionally substituted with one or two —COOH;
      each R^{1K} is independently H or C₁₋₃alkyl, wherein the C₁₋₃alkyl is optionally substituted with one or two —COOH;
      each R^{1M} is independently H or C₁₋₄alkyl, wherein the C₁₋₄alkyl is optionally substituted with phenyl;
      each R^{1N} is independently H or C₁₋₃alkyl;
      each R^{1O} is independently H or C₁₋₃alkyl;
      each R^{1W} is independently H or C₁₋₃alkyl;
      e is 1, 2, or 3;
each R^{1X} is H or C₁₋₄alkyl; wherein the C₁₋₄alkyl is optionally substituted with one or two —COOH; and
each R^{1Y} is H or C₁₋₄alkyl; wherein the C₁₋₄alkyl is optionally substituted with one or two —COOH.

In some embodiments, X is phenyl or pyridyl; wherein the phenyl or pyridyl is optionally substituted with one, two or three substituents independently selected from the group consisting of —OP(O)(OR^{1E})₂, R^{1F}, —COOH, —OCO—C₁₋₂₀alkyl, —NHCO—C₁₋₂₀alkyl,
wherein each R^{1E} is independently H or phenyl;
each R^{1F} is independently a C₁₋₃alkyl optionally substituted with one or two substituents independently selected from the group consisting of —COOH, —P(O)(OH)₂, —OP(O)(OR^{1E})₂, —NR^{1G}R^{1H}, —CONR^{1G}R^{1H}, —OCOR^{1I}, and —NHCOR^{1I};
   each R^{1G} is independently H, —COO(CR^{1N}R^{1O})ₑOP(O)(OH)₂, or C₁₋₄alkyl; wherein the C₁₋₄ alkyl is optionally substituted with one or two substituents independently selected from a group consisting of —COOR^{1M}, —OH, —NH₂, —NR^{1J}R^{1K}, and —CONR^{1J}R^{1K}; each R^{1H} is independently H, —COO(CR^{1N}R^{1O})ₑOP(O)(OH)₂, or C₁₋₄alkyl; wherein the C₁₋₄ alkyl is optionally substituted with one or two substituents independently selected from a group consisting of —COOR^{1M}, —OH, —NH₂, —NR^{1J}R^{1K}, and —CONR^{1J}R^{1K}; or
   optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
   each R^{1I} is independently C₁₋₂₀alkyl optionally substituted with one or two substituents selected independently from —COOH and —NR^{1J}R^{1K};
      each R^{1J} is independently H or C₁₋₃alkyl, wherein the C₁₋₃alkyl is optionally substituted with one or two —COOH;
      each R^{1K} is independently H or C₁₋₃alkyl, wherein the C₁₋₃alkyl is optionally substituted with one or two —COOH;
      each R^{1M} is independently H or C₁₋₄alkyl, wherein the C₁₋₃alkyl is optionally substituted with phenyl;
      each R^{1N} is independently H or C₁₋₃alkyl;
      each R^{1O} is independently H or C₁₋₃alkyl; and
      e is 1, 2, or 3.

In some embodiments, X is phenyl; wherein the phenyl is optionally substituted with one, two or three substituents independently selected from the group consisting of —OP(O)(OR^{1E})₂, R^{1F}, —COOH, —OCO—C₁₋₂₀alkyl, CONR^{1X}R^{1Y}, and —NHCO—C₁₋₂₀alkyl,
wherein each R^{1E} is independently H or phenyl;
each R^{1F} is independently a C₁₋₃alkyl optionally substituted with one or two substituents independently selected from the group consisting of —COOH, —P(O)(OH)₂, —OP(O)(OR^{1E})₂, —NR^{1G}R^{1H}, —CONR^{1G}R^{1H}, —OCOR^{1I}, and —NHCOR^{1I};
   each R^{1G} is independently H, —COOCH₂OP(O)(OH)₂, or C₁₋₄alkyl; wherein the C₁₋₄ alkyl is optionally substituted with one, two, or three substituents independently selected from a group consisting of halo, —COOH, —OR^{1W}, —P(O)(OH)₂, —NH₂, —CONH₂ and —NR^{1J}R^{1K};
   each R^{1H} is independently H, —COOCH₂OP(O)(OH)₂, or C₁₋₄alkyl; wherein the C₁₋₄ alkyl is optionally substituted with one, two, or three substituents independently selected from a group consisting of halo, —COOH, —OR^{1W}, —P(O)(OH)₂, —NH₂, —CONH₂ and —NR^{1J}R^{1K}; or
   optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
   each R^{1I} is independently C₁₋₂₀alkyl optionally substituted with one or two substituents selected independently from —P(O)(OH)₂, —COOH and —NR^{1J}R^{1K};
      each R^{1J} is independently H or C₁₋₃alkyl wherein the C₁₋₃alkyl is optionally substituted with one or two —COOH;
      each R^{1K} is independently H or C₁₋₃alkyl wherein the C₁₋₃alkyl is optionally substituted with one or two —COOH;
      each R^{1W} is independently H or C₁₋₃alkyl;
each R^{1X} is H or C₁₋₄alkyl; wherein the C₁₋₄alkyl is optionally substituted with one or two —COOH; and
each R^{1Y} is H or C₁₋₄alkyl; wherein the C₁₋₄alkyl is optionally substituted with one or two —COOH.

In some embodiments, X is phenyl; wherein the phenyl is optionally substituted with one, two or three substituents independently selected from the group consisting of —OP(O)(OR^{1E})₂, R^{1F}, —COOH, —OCO—C₁₋₂₀alkyl, —NHCO—C₁₋₂₀alkyl,
wherein each R^{1E} is independently H or phenyl;
each R^{1F} is independently a C₁₋₃alkyl optionally substituted with one or two substituents independently selected from the group consisting of —COOH, —P(O)(OH)₂, —OP(O)(OR^{1E})₂, —NR^{1G}R^{1H}, —CONR^{1G}R^{1H}, —OCOR^{1I}, and —NHCOR^{1I};
   each R^{1G} is independently H, —COOCH₂OP(O)(OH)₂, or C₁₋₄alkyl; wherein the C₁₋₄ alkyl is optionally substituted with one or two substituents independently selected from a group consisting of —COOH, —OH, —NH₂, —CONH₂ and —NR^{1J}R^{1K};
   each R^{1H} is independently H, —COOCH₂OP(O)(OH)₂, or C₁₋₄alkyl; wherein the C₁₋₄ alkyl is optionally substituted with one or two substituents independently selected from a group consisting of —COOH, —OH, —NH₂, —CONH₂ and —NR^{1J}R^{1K}; or
   optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
   each R^{1I} is independently C₁₋₂₀alkyl optionally substituted with one or two substituents selected independently from —COOH and —NR^{1J}R^{1K};
      each R^{1J} is independently H or C₁₋₃alkyl; and
      each R^{1K} is independently H or C₁₋₃alkyl.

In some embodiments, X is phenyl; wherein the phenyl is optionally substituted with one, two or three substituents independently selected from the group consisting of —OP(O)(OR^{1E})₂, R^{1F}, —COOH, —OCO—C₁₋₂₀alkyl, —NHCO—C₁₋₂˜alkyl;
each R^{1F} is independently a —CH₃ optionally substituted with one or two substituents independently selected from the group consisting of —COOH, —P(O)(OH)₂, —OP(O)(OR^{1E}), —NR^{1G}R^{1H}, —CONR^{1G}R^{1H}, —OCOR^{1I}, and —NHCOR^{1I};
   each R^{1G} is independently H, —COOCH₂OP(O)(OH)₂, or C₁₋₄alkyl; wherein the C₁₋₄ alkyl is optionally substituted with one or two substituents independently selected from a group consisting of —COOH, —OH, —NH₂, —CONH₂ and —NR^{1J}R^{1K};
   each R^{1H} is independently H, —COOCH₂OP(O)(OH)₂, or C₁₋₄alkyl; wherein the C₁₋₄alkyl is optionally substituted with one or two substituents independently selected from a group consisting of —COOH, —OH, —NH₂, —CONH₂ and —NR^{1J}R^{1K}; or
   optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
   each R^{1I} is independently C₁₋₂₀alkyl optionally substituted with one or two substituents selected independently from —COOH and —NR^{1J}R^{1K};
      each R^{1J} is independently H or CH₃; and
      each R^{1K} is independently H or CH₃.

In some embodiments, X is phenyl; wherein the phenyl is optionally substituted with one, two or three substituents independently selected from the group consisting of —OP(O)(OH)₂, R^{1F} and —COOH;
each R^{1F} is independently a —CH₃ optionally substituted with one or two substituents independently selected from the group consisting of —COOH, —P(O)(OH)₂, —OP(O)(OH), —NR^{1G}R^{1H}, —CONR^{1G}R^{1H} and —OCOR^{1I};
   each R^{1G} is independently H or —COOCH₂OP(O)(OH)₂;
   each R^{1H} is independently C₁₋₄alkyl; wherein the C₁₋₄ alkyl is optionally substituted with one or two —COOH groups;
   each R^{1I} is independently C₁₋₆alkyl optionally substituted with one or two substituents selected independently from —COOH and —NR^{1J}R^{1K};
      each R^{1J} is independently H or CH₃; and
      each R^{1K} is independently H or CH₃.

In some embodiments, X is phenyl; wherein the phenyl is optionally substituted with one, two or three substituents independently selected from the group consisting —OP(O)(OH)₂, R^{1F}, —OCO—C₁₋₂₀ alkyl, —COOH, and —NHCO—C₁₋₂₀alkyl.
each R^{1F} is independently a —CH₃ optionally substituted with one or two substituents independently selected from the group consisting of —COOH, —CONR^{1G}R^{1H}, —OCOR^{1I}, and —NHCOR^{1I};
each R^{1G} is independently H;
each R^{1H} is independently C₁₋₄alkyl; wherein the C₁₋₄ alkyl is optionally substituted with one or two substituents independently selected from a group consisting of —COOH and —P(O)(OH)₂; or
optionally R^{1G} and R^{1H} are joined to form a 5 to 6 membered heterocycle comprising 1 or 2 heteroatoms selected from N and O; wherein the 4 to 6 membered heterocycle is optionally substituted with —COOH; and
each R^{1I} is independently C₁₋₂₀ alkyl.

In some embodiments, X is phenyl; w % herein the phenyl is optionally substituted with one, two or three substituents independently selected from the group consisting —OP(O)(OH)₂, R^{1F}, and —COOH,
each R^{1F} is independently a —CH₃ optionally substituted with one or two substituents independently selected from the group consisting of —COOH and —CONR^{1G}R^{1H};
   each R^{1G} is independently H; and
   each R^{1H} is independently C₁₋₄alkyl; wherein the C₁₋₄ alkyl is optionally substituted with one or two substituents independently selected from a group consisting of —COOH and —P(O)(OH)₂; or
   optionally R^{1G} and R^{1H} are joined to form a 5 to 6 membered heterocycle comprising 1 or 2 heteroatoms selected from N and O; wherein the 4 to 6 membered heterocycle is optionally substituted with —COOH.

In some embodiments, X is phenyl.

In some embodiments, X is pyridyl; wherein the pyridyl is optionally substituted with one, two or three substituents independently selected from the group consisting of —OP(O)(OH)₂, R^{1F}—COOH, and —CONR^{1X}R^{1Y};
each R^{1F} is independently a C₁₋₃alkyl optionally substituted with one or two substituents independently selected from the group consisting of —COOH, —P(O)(OH)₂, —OP(O)(OH)₂, —NR^{1G}R^{1H}, —CONR^{1G}R^{1H} and —OCOR^{1I},
   each R^{1G} is independently H or —COO(CR^{1N}R^{1O})ₑOP(O)(OH)₂;
   each R^{1H} is independently CH₃ optionally substituted with one or two substituents independently selected from a group consisting of halo, —COOR^{1M}, —OR^{1W}, and —CONR^{1J}R^{1K};
   each R^{1I} is independently C₁₋₆alkyl optionally substituted with one or two substituents selected independently from —COOH and —NR^{1J}R^{1K},
      each R^{1J} is independently H or CH₃;
      each R^{1K} is independently H or C₁₋₃alkyl, wherein the C₁₋₃alkyl is optionally substituted with one or two —COOH;
      each R^{1M} is independently H, CH₃, tert-butyl, or benzyl;
      each R^{1N} is independently H or CH₃;
      each R^{1O} is independently H or CH₃;
      each R^{1W} is independently H or CH₃;
      e is 1 or 2;
each R^{1X} is H or C₁₋₄alkyl; wherein the C₁₋₄alkyl is optionally substituted with one or two —COOH, and
each R^{1Y} is H or C₁₋₄alkyl; wherein the C₁₋₄alkyl is optionally substituted with one or two —COOH.

In some embodiments, X is pyridyl; wherein the pyridyl is optionally substituted with one or two substituents independently selected from the group consisting of —OP(O)(OH)₂, R^{1F} and —COOH, and —CONR^{1X}R^{1Y};
each R^{1F} is independently a —CH₃ optionally substituted with one or two substituents independently selected from the group consisting of —OP(O)(OH)₂, —NR^{1G}R^{1H}, and —OCOR^{1I};
   each R^{1G} is independently H or —COOCH₂OP(O)(OH)₂;
   each R^{1H} is independently CH₃ optionally substituted with one or two substituents independently selected from a group consisting of halo, —COOR^{1M}, —OR^{1W}, and —CONR^{1J}R^{1K};
   each R^{1I} is independently C₁₋₆alkyl optionally substituted with one or two substituents selected independently from —COOH and —NR^{1J}R^{1K};
      each R^{1J} is independently H;
      each R^{1K} is independently H or C₁₋₃alkyl, wherein the C₁₋₃alkyl is optionally substituted with one or two —COOH;
      each R^{1M} is independently H, CH₃, tert-butyl, or benzyl;
      each R^{1W} is independently H or CH₃;
each R^{1X} is H; and
each R^{1Y} is C₁₋₄alkyl; wherein the C₁₋₄alkyl is optionally substituted with one or two —COOH.

In some embodiments, X is pyridyl; wherein the pyridyl is optionally substituted with one, two or three substituents independently selected from the group consisting of —OP(O)(OH)₂, R^{1F} and —COOH;
each R^{1F} is independently a —CH₃ optionally substituted with one or two substituents independently selected from the group consisting of —COOH, —P(O)(OH)₂, —OP(O)(OH)₂, —NR^{1G}R^{1H}, —CONR^{1G}R^{1H} and —OCOR^{1I};
   each R^{1G} is independently H or —COO(CR^{1N}R^{1O})ₑOP(O)(OH)₂;
   each R^{1H} is independently CH₃ optionally substituted with one or two —COOR^{1M} groups;
   each R^{1I} is independently C₁₋₆alkyl optionally substituted with one or two substituents selected independently from —COOH and —NR^{1J}R^{1K};
      each R^{1J} is independently H or CH₃;
      each R^{1K} is independently H or CH₃;
      each R^{1M} is independently H, CH₃, tert-butyl, or benzyl; and
      each R^{1N} is independently H or CH₃;
      each R^{1O} is independently H or CH₃; and
      e is 1 or 2.

In some embodiments, X is pyridyl; wherein the pyridyl is optionally substituted with one, two or three substituents independently selected from the group consisting of —OP(O)(OH)₂, RIF and —COOH;
each R^{1F} is independently a —CH₃ optionally substituted with one or two substituents independently selected from the group consisting of —COOH, —P(O)(OH)₂, —OP(O)(OH)₂, —NR^{1G}R^{1H}, —CONR^{1G}R^{1H} and —OCOR^{1I};
   each R^{1G} is independently H, —COO(CR^{1N}R^{1O})ₑOP(O)(OH)₂, or C₁₋₄alkyl; wherein the C₁₋₄ alkyl is optionally substituted with one or two substituents independently selected from a group consisting of —COOR^{1M}, —OH, —NH₂, —NR^{1J}R^{1K}, and —CONR^{1J}R^{1K};
   each R^{1H} is independently H, —COO(CR^{1N}R^{1O})ₑOP(O)(OH)₂, or C₁₋₄alkyl; wherein the C₁₋₄ alkyl is optionally substituted with one or two substituents independently selected from a group consisting of —COOR^{1M}, —OH, —NH₂, —NR^{1J}R^{1K}, and —CONR^{1J}R^{1K};
   each R^{1I} is independently C₁₋₆alkyl optionally substituted with one or two substituents selected independently from —COOH and —NR^{1J}R^{1K};
      each R^{1J} is independently H, CH₃, or —CH(CH₂COOH)₂;
      each R^{1K} is independently H, CH₃, or —CH(CH₂COOH)₂;
      each R^{1M} is independently H, CH₃, tert-butyl, or benzyl;
      each R^{1N} is independently H or CH₃;
      each R^{1O} is independently H or CH₃; and
      e is 1 or 2.

In some embodiments, X is pyridyl; wherein the pyridyl is optionally substituted with one, two or three substituents independently selected from the group consisting of —OP(O)(OH)₂, R^{1F} and —COOH;
each R^{1F} is independently a —CH₃ optionally substituted with one or two substituents independently selected from the group consisting of —COOH, —P(O)(OH)₂, —OP(O)(OH)₂, —NR^{1G}R^{1H}, —CONR^{1G}R^{1H} and —OCOR^{1I};
   each R^{1G} is independently H or —COOCH₂OP(O)(OH)₂;
   each R^{1H} is independently C₁₋₄alkyl; wherein the C₁₋₄ alkyl is optionally substituted with one or two —COOR^{1M} groups;
   each R^{1I} is independently C₁₋₆alkyl optionally substituted with one or two substituents selected independently from —COOH and —NR^{1J}R^{1K};
      each R^{1J} is independently H or CH₃; and
      each R^{1K} is independently H or CH₃; and
      each R^{1M} is independently H, CH₃, tert-butyl, or benzyl.

In some embodiments, X is pyridyl; wherein the pyridyl is optionally substituted with one, two or three substituents independently selected from the group consisting of —OP(O)(OH)₂, RIF and —COOH,
each R^{1F} is independently a —CH₃ optionally substituted with one or two substituents independently selected from the group consisting of —COOH, —P(O)(OH)₂, —OP(O)(OH)₂, —NR^{1G}R^{1H}, —CONR^{1G}R^{1H} and —OCOR^{1I};
   each R^{1G} is independently H or —COOCH₂OP(O)(OH)₂;
   each R^{1H} is independently CH₃ optionally substituted with one or two —COOR^{1M} groups;
   each R^{1I} is independently C₁₋₆alkyl optionally substituted with one or two substituents selected independently from —COOH and —NR^{1J}R^{1K};
      each R^{1J} is independently H or CH₃;
      each R^{1K} is independently H or CH₃; and
      each R^{1M} is independently H, CH₃, tert-butyl, or benzyl.

In some embodiments, X is 4 to 7 membered heterocyclyl comprising 1, 2 or 3 heteroatoms selected from N, O, and S; wherein the heterocyclyl is optionally substituted with one, two or three substituents independently selected from the group consisting of C₁₋₄alkyl, —COOC₁₋₄alkyl, oxo, and —COOCH₂OP(O)(OH)₂, wherein the C₁₋₄alkyl is optionally substituted with —OP(O)(OH)₂ or —NR^{1Z}R^{1AA};
wherein R^{1Z} is H, —COO(CR^{1AB}R^{1AC})ₕOP(O)(OH)₂ or C₁₋₄alkyl wherein the C₁₋₄ alkyl is optionally substituted with one or two —COOH;
R^{1AA} is H, —COO(CR^{1AD}R^{1AE})ᵢOP(O)(OH)₂ or C₁₋₄alkyl wherein the C₁₋₄ alkyl is optionally substituted with one or two —COOH;
   each R^{1AB} is independently H or C₁₋₃alkyl;
   each R^{1AC} is independently H or C₁₋₃alkyl;
   each R^{1AD} is independently H or C₁₋₃alkyl;
   each R^{1AE} is independently H or C₁₋₃alkyl;
   h is 1, 2, or 3; and
   i is 1, 2, or 3.

In some embodiments, X is a 4 to 7 membered heterocyclyl comprising 1, 2 or 3 heteroatoms selected from N and O; wherein the heterocyclyl is optionally substituted with one, two or three substituents independently selected from the group consisting of C₁₋₄alkyl, —COOC₁₋₄alkyl, oxo, and —COOCH₂OP(O)(OH)₂, wherein the C₁₋₄alkyl is optionally substituted with —OP(O)(OH)₂ or —NR^{1Z}R^{1AA};
wherein R^{1Z} is H or C₁₋₄alkyl, wherein the C₁₋₄alkyl is optionally substituted with one or two —COOH; and
R^{1AA} is —COOCH₂OP(O)(OH)₂.

In some embodiments, X is a 4 to 6 membered heterocyclyl comprising 1, 2 or 3 heteroatoms selected from N and O; wherein the heterocyclyl is optionally substituted with one or two substituents independently selected from the group consisting of —CH₃, oxo, and —COOCH₂OP(O)(OH)₂, wherein the —CH₃ is optionally substituted with —OP(O)(OH)₂ or —NR^{1Z}R^{1AA};
wherein R^{1Z} is CH₂COOH; and
R^{1AA} is —COOCH₂OP(O)(OH)₂.

In some embodiments, X is 4 to 7 membered heterocyclyl comprising 1, 2 or 3 heteroatoms selected from N, O, and S; wherein the heterocyclyl is optionally substituted with one, two or three substituents independently selected from the group consisting of C₁₋₄alkyl, —COOC₁₋₄alkyl, oxo, and —COOCH₂OP(O)(OH)₂, wherein the C₁₋₄alkyl is optionally substituted with —OP(O)(OH)₂. In some embodiments. X is a 4 to 7 membered heterocyclyl comprising 1, 2 or 3 heteroatoms selected from N and O; wherein the heterocyclyl is optionally substituted with one, two or three substituents independently selected from the group consisting of C₁₋₄ alkyl, —COOC₁₋₄alkyl; oxo and —COOCH₂OP(O)(OH)₂, wherein the C₁₋₄alkyl is optionally substituted with —OP(O)(OH)₂.

In some embodiments, X is a 5 membered heterocyclyl comprising 1, 2 or 3 heteroatoms selected from N and O; wherein the heterocyclyl is optionally substituted with one, two or three substituents independently selected from the group consisting of C₁₋₃alkyl, oxo and —COOCH₂OP(O)(OH)₂. In some embodiments, X is a 5 membered heterocyclyl comprising 1 or 2 heteroatoms selected from N and O; wherein the heterocyclyl is optionally substituted with one, two or three substituents independently selected from the group consisting of C₁₋₃alkyl, oxo and —COOCH₂OP(O)(OH)₂.

In some embodiments, X is 4 to 7 membered heterocyclyl comprising 1, 2 or 3 heteroatoms selected from N, O, and S; wherein the heterocyclyl is optionally substituted with one, two or three substituents independently selected from the group consisting of —CH₃, oxo, —COOCH₂OP(O)(OH)₂, and —CH₂OP(O)(OH)₂. In some embodiments, X is a 4 to 7 membered heterocyclyl comprising 1, 2 or 3 heteroatoms selected from N and O; wherein the heterocyclyl is optionally substituted with one, two or three substituents independently selected from the group consisting of —CH₃, oxo, —COOCH₂OP(O)(OH)₂, and —CH₂OP(O)(OH)₂.

In some embodiments, X is a 5 membered heterocyclyl comprising 1, 2 or 3 heteroatoms selected from N and O; wherein the heterocyclyl is optionally substituted with one, two or three substituents independently selected from the group consisting of —CH₃, oxo and —COOCH₂OP(O)(OH)₂. In some embodiments, X is a 5 membered heterocyclyl comprising 1 or 2 heteroatoms selected from N and O; wherein the heterocyclyl is optionally substituted with one, two or three substituents independently selected from the group consisting of —CH₃, oxo and —COOCH₂OP(O)(OH)₂.

In some embodiments, X is a 4 to 6 membered heterocyclyl comprising 1 or 2 heteroatoms selected from N, O, and S; wherein the heterocyclyl is optionally substituted with one, two, or three substituents independently selected from the group consisting of C₁₋₄alkyl, —COOC₁₋₄alkyl, oxo, and —CH₂OP(O)(OH)₂. In some embodiments, X is a 6 membered heterocyclyl comprising 1 or 2 heteroatoms selected from N and O; wherein the heterocyclyl is optionally substituted with —CH₂OP(O)(OH)₂.

In some embodiments, X is a 4 to 6 membered heterocyclyl comprising 1 or 2 heteroatoms selected from N and O; wherein the heterocyclyl is optionally substituted with one, two, or three substituents independently selected from the group consisting of C₁₋₁₄alkyl, —COOC₁₋₄alkyl, oxo, —COOCH₂OP(O)(OH)₂, —CH₂OP(O)(OH)₂, and —N(CH₂COOH)(—COOCH₂OP(O)(OH)₂.

In some embodiments, X is 5 to 7 membered heterocyclyl comprising 1, 2 or 3 heteroatoms selected from N, O, and S. In some embodiments, X is a 5 to 7 membered heterocyclyl comprising 1, 2 or 3 heteroatoms selected from N and 0.

In some embodiments, X is a 5 membered heterocyclyl comprising 1, 2 or 3 heteroatoms selected from N and O. In some embodiments, X is a 5 membered heterocyclyl comprising 1 or 2 heteroatoms selected from N and 0.

In some embodiments, X is C₃₋₇ cycloalkyl optionally substituted with one, two or three substituents independently selected from the group consisting of C₁₋₄alkyl, —COOC₁₋₄alkyl, oxo, —COOR^{1P}, and —OP(O)(OH)₂, wherein the C₁₋₄alkyl is optionally substituted with —OP(O)(OH)₂ or —NR^{1Q}R^{1R};
wherein R^{1P} is H or C₁₋₃ alkyl;
R^{1Q} is H, —COO(CR^{1S}R^{1T})_{f}OP(O)(OH)₂, or C₁₋₄alkyl; wherein the C₁₋₄alkyl is optionally substituted with —COOH;
R^{1R} is H, —COO(CR^{1U}R^{1V})_{g}OP(O)(OH)₂, or C₁₋₄alkyl; wherein the C₁₋₄alkyl is optionally substituted with —COOH;
   each R^{1S}, R^{1T}, R^{1U}, and R^{1V} is independently H or C₁₋₃alkyl;
   f is 1, 2, or 3; and
   g is 1, 2, or 3.

In some embodiments, X is C₃₋₇ cycloalkyl optionally substituted with one, two or three substituents independently selected from the group consisting of C₁₋₄alkyl, —COOC₁₋₄alkyl, oxo, —COOH, and —OP(O)(OH)₂, wherein the C₁₋₄alkyl is optionally substituted with —OP(O)(OH)₂ or —NR^{1Q}R^{1R};
wherein R^{1Q} is H, or C₁₋₄alkyl; wherein the C₁₋₄alkyl is optionally substituted with —COOH; and
R^{1R} is —COOCH₂OP(O)(OH)₂.

In some embodiments, X is C₃₋₇ cycloalkyl optionally substituted with one or two substituents independently selected from the group consisting of —CH₃, —COOH, and —OP(O)(OH)₂, wherein the —CH₃ is optionally substituted with —OP(O)(OH)₂ or —NR^{1Q}R^{1R},
wherein R^{1Q} is —CH₂COOH; and
R^{1R} is —COOCH₂OP(O)(OH)₂.

In some embodiments, X is C₃₋₆ cycloalkyl optionally substituted with one, two or three substituents independently selected from the group consisting of C₁₋₄alkyl, —COOC₁₋₄alkyl, oxo, and —OP(O)(OH)₂, wherein the C₁₋₄alkyl is optionally substituted with —OP(O)(OH)₂.

In some embodiments, each R^{1E} is H.

In some embodiments, each R^{1F} is independently a —CH₃ optionally substituted with one or two substituents independently selected from the group consisting of —COOH, —P(O)(OH)₂, —OP(O)(OH)₂, —NR^{1G}R^{1H}, —CONR^{1G}R^{1H}, —OCOR^{1I}, and —NHCOR^{1I}. In some embodiments, each R^{1F} is independently a —CH₃ optionally substituted with one or two substituents independently selected from the group consisting of —COOH, —P(O)(OH)₂, —OP(O)(OH)₂, —NR^{1G}R^{1H}, —CONR^{1G}R^{1H} and —OCOR^{1I}.

In some embodiments, R^{1F} is —CH₂—OCO—C₁₋₂₀alkyl. In some embodiments, R^{1F} is —CH₂—NHCO—C₁₋₂₀alkyl.

In some embodiments, R^{1G} and R^{1H} are each independently H, —COO(CR^{1N}R^{1O})ₑOP(O)(OH)₂, or C₁₋₂alkyl, wherein the C₁₋₂alkyl is optionally substituted with one, two, or three substitutents independently selected from a group consisting of halo, —COOR^{1M}, —OR^{1W}, —P(O)(OH)₂, and —CONR^{1J}R^{1K}.

In some embodiments, each R^{1G} is independently H, —COO(CR^{1N}R^{1O})ₑOP(O)(OH)₂, —CONR^{1J}R^{1K}, or C₁₋₄alkyl substituted with one or two —COOH. In some embodiments, each R^{1G} is independently H, —COOCH₂OP(O)(OH)₂, —CONHCH(CH₂COOH)₂, or C₂₋₃alkyl substituted with two —COOH.

In some embodiments, each R^{1G} is independently H or —COO(CR^{1N}R^{1O})ₑOP(O)(OH)₂. In some embodiments, each R^{1G} is independently H or —COOCH₂OP(O)(OH)₂.

In some embodiments, each R^{1H} is independently C₁₋₄alkyl; wherein the C₁₋₄ alkyl is optionally substituted with one or two substituents independently selected from the group consisting of —COOR^{1M} and —OH. In some embodiments, each R^{1H} is independently CH₃ optionally substituted with one or two substituents independently selected from the group consisting of —COOR^{1M} and —OH.

In some embodiments, each R^{1H} is independently C₁₋₄alkyl; wherein the C₁₋₄ alkyl is optionally substituted with one or two —COOR^{1M} groups. In some embodiments, each R^{1H} is independently CH₃ optionally substituted with one or two —COOR^{1M} groups.

In some embodiments, each R^{1H} is independently C₁₋₄alkyl; wherein the C₁₋₄ alkyl is optionally substituted with one or two —COOH groups. In some embodiments, each R^{1H} is independently CH₃ optionally substituted with one or two —COOH groups.

In some embodiments, R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH. In some embodiments, the 4 to 6 membered heterocycle comprises at least one N heteroatom.

In some embodiments, R^{1G} and R^{1H} are joined to form a 4 to 6 membered heterocycle comprising 1, 2, or 3 heteroatoms selected from N and O; wherein the 4 to 6 membered heterocycle is optionally substituted with one or two substituents independently selected from the group consisting of —OH and —COOH. In some embodiments, R^{1G} and R^{1H} are joined to form a 5 to 6 membered heterocycle comprising 1 or 2 heteroatoms selected from N and O; wherein the 5 to 6 membered heterocycle is optionally substituted with one or two substituents independently selected from the group consisting of —OH and —COOH.

In some embodiments, each R^{1I} is independently C₁₋₂₀alkyl. In some embodiments, R^{1I} is C₁₀₋₂₀alkyl. In some embodiments, R^{1I} is C₁₅₋₂₀alkyl.

In some embodiments, each R^{1J} is independently H or CH₃.

In some embodiments, each R^{1K} is independently H or CH₃.

In some embodiments, each R^{1J} is independently H or CH₃ and each R^{1K} is independently H or CH₃.

In some embodiments, each R^{1M} is independently H, CH₃, tert-butyl, or benzyl. In some embodiments, each R^{1M} is H. In some embodiments, each R^{1M} is CH₃. In some embodiments, each R^{1M} is tert-butyl. In some embodiments, each R^{1M} is benzyl.

In some embodiments, each R^{1N} is independently H or CH₃.

In some embodiments, each R^{1O} is independently H or CH₃.

In some embodiments, each R^{1N} is independently H or CH₃ and each R^{1O} is independently H or CH₃. In some embodiments, each R^{1N} is H or CH₃ and each R^{1O} is H.

In some embodiments, each R^{1W} is independently H or CH₃.

In some embodiments, R^{1X} is H.

In some embodiments, R^{1Y} is C₁₋₄alkyl, wherein the C₁₋₄alkyl is optionally substituted with one or two —COOH.

In some embodiments, R^{1Z} is H or C₁₋₄alkyl, wherein the C₁₋₄alkyl is optionally substituted with one or two —COOH. In some embodiments, R^{1Z} is CH₂COOH.

In some embodiments, R^{1AA} is —COOCH₂OP(O)(OH)₂.

In some embodiments, each R^{1AB} is independently H or CH₃. In some embodiments, each R^{1AB} is H.

In some embodiments, each R^{1AC} is independently H or CH₃. In some embodiments, each R^{1AC} is H.

In some embodiments, each R^{1AD} is independently H or CH₃. In some embodiments, each R^{1AD} is H.

In some embodiments, each R^{1AE} is independently H or CH₃. In some embodiments, each R^{1AE} is H.

In some embodiments, each R^{1P} is H or CH₃. In some embodiments, each R^{1P} is H.

In some embodiments, each R^{1Q} is H or C₁₋₄alkyl, wherein the C₁₋₄alkyl is optionally substituted with —COOH. In some embodiments, R^{1Q} is —CH₂COOH.

In some embodiments, each R^{1R} is H or —COOCH₂OP(O)(OH)₂. In some embodiments, R^{1R} is —COOCH₂OP(O)(OH)₂.

In some embodiments, each R^{1S}, R^{1T}, R^{1U}, and R^{1V} is H.

In some embodiments, R¹ is selected from the group consisting of:

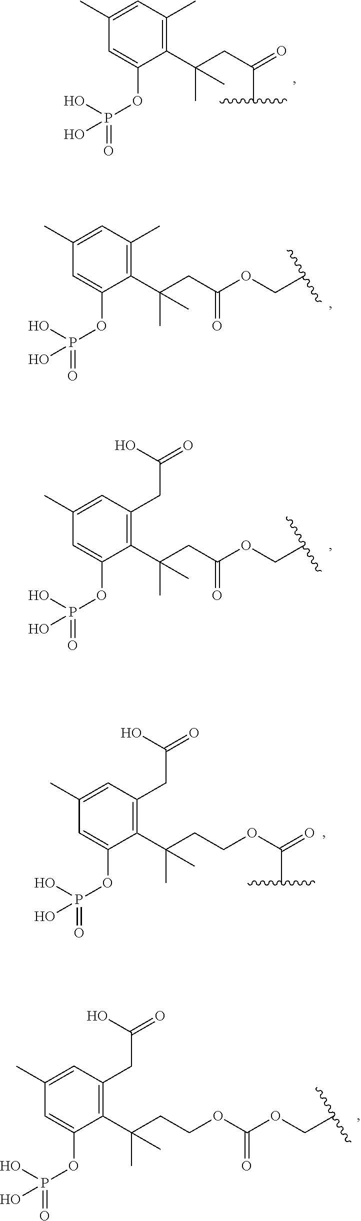 [SMILES: *C(=O)CC(C)(C)c1c(C)cc(C)cc1OP(=O)(O)O ; *COC(=O)CC(C)(C)c1c(C)cc(C)cc1OP(=O)(O)O ; *COC(=O)CC(C)(C)c1c(CC(=O)O)cc(C)cc1OP(=O)(O)O ; *C(=O)OCCC(C)(C)c1c(CC(=O)O)cc(C)cc1OP(=O)(O)O ; *COC(=O)OCCC(C)(C)c1c(CC(=O)O)cc(C)cc1OP(=O)(O)O]

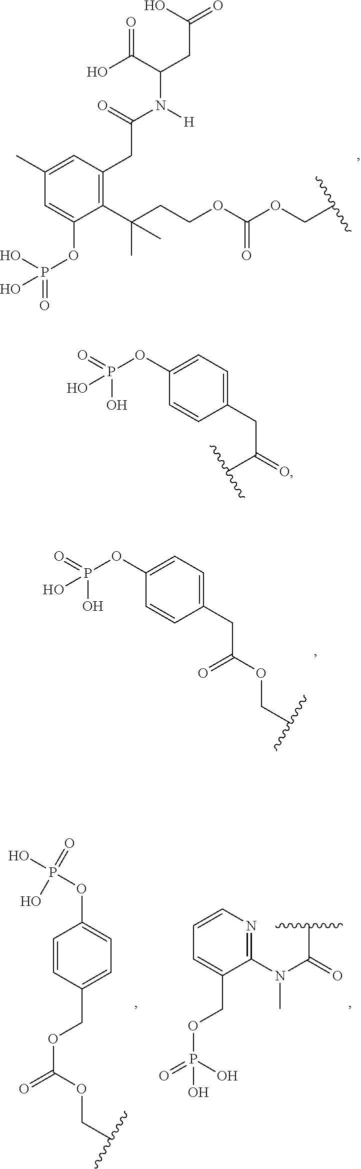 [SMILES: *COC(=O)OCCC(C)(C)c1c(CC(=O)NC(CC(=O)O)C(=O)O)cc(C)cc1OP(=O)(O)O ; *C(=O)Cc1ccc(OP(=O)(O)O)cc1 ; *COC(=O)Cc1ccc(OP(=O)(O)O)cc1 ; *COC(=O)OCc1ccc(OP(=O)(O)O)cc1 ; *C(=O)N(C)c1ncccc1COP(=O)(O)O]

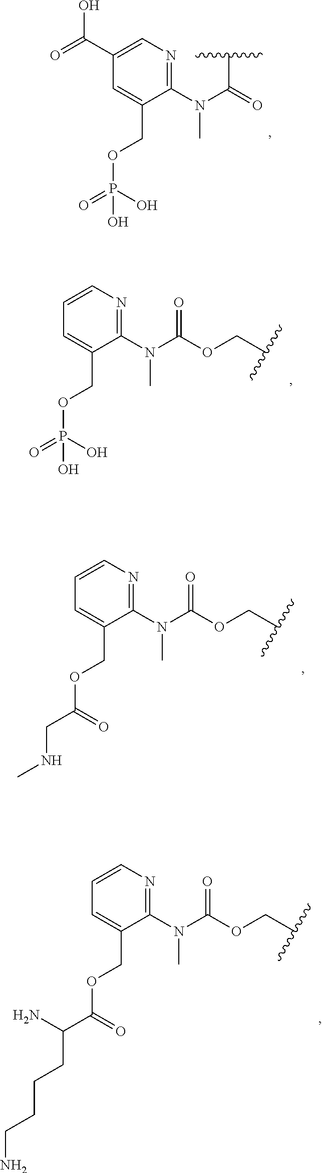 [SMILES: *C(=O)N(C)c1ncc(C(=O)O)cc1COP(=O)(O)O ; *COC(=O)N(C)c1ncccc1COP(=O)(O)O ; *COC(=O)N(C)c1ncccc1COC(=O)CNC ; *COC(=O)N(C)c1ncccc1COC(=O)C(N)CCCCN]

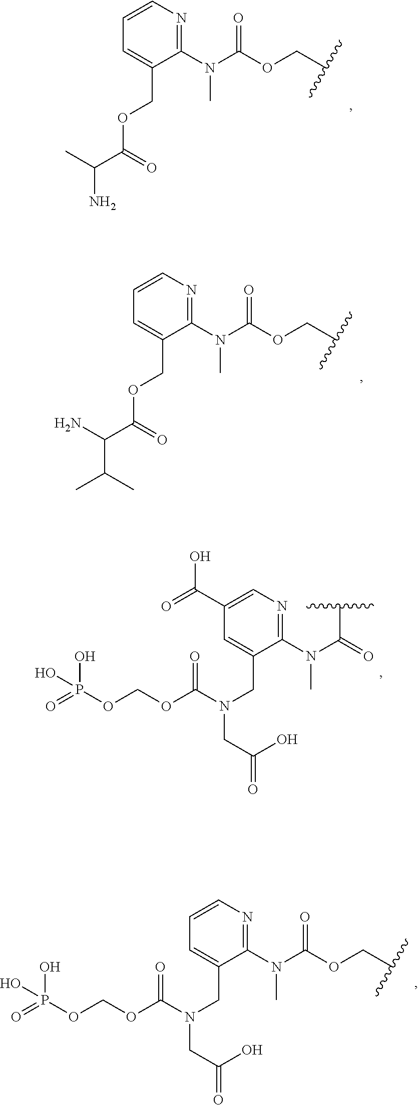 [SMILES: *COC(=O)N(C)c1ncccc1COC(=O)C(C)N ; *COC(=O)N(C)c1ncccc1COC(=O)C(N)C(C)C ; *C(=O)N(C)c1ncc(C(=O)O)cc1CN(CC(=O)O)C(=O)OCOP(=O)(O)O ; *COC(=O)N(C)c1ncccc1CN(CC(=O)O)C(=O)OCOP(=O)(O)O]

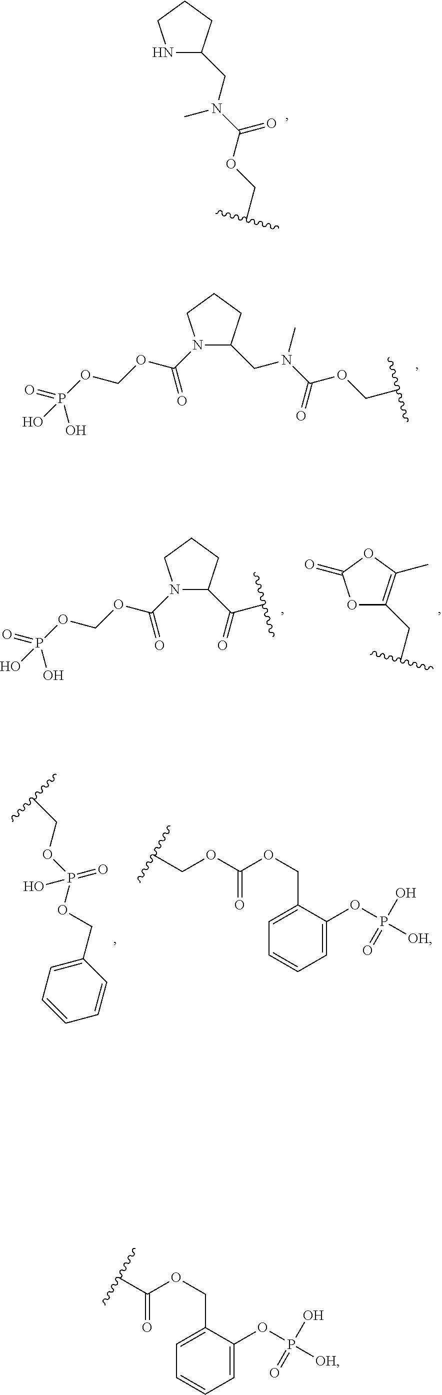

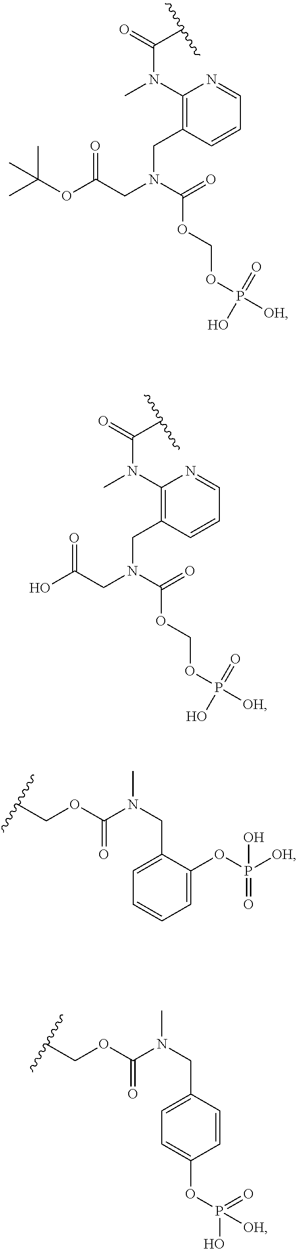 [SMILES: *C(=O)N(C)c1ncccc1CN(CC(=O)OC(C)(C)C)C(=O)OCOP(=O)(O)O ; *C(=O)N(C)c1ncccc1CN(CC(=O)O)C(=O)OCOP(=O)(O)O ; *COC(=O)N(C)Cc1ccccc1OP(=O)(O)O ; *COC(=O)N(C)Cc1ccc(OP(=O)(O)O)cc1]

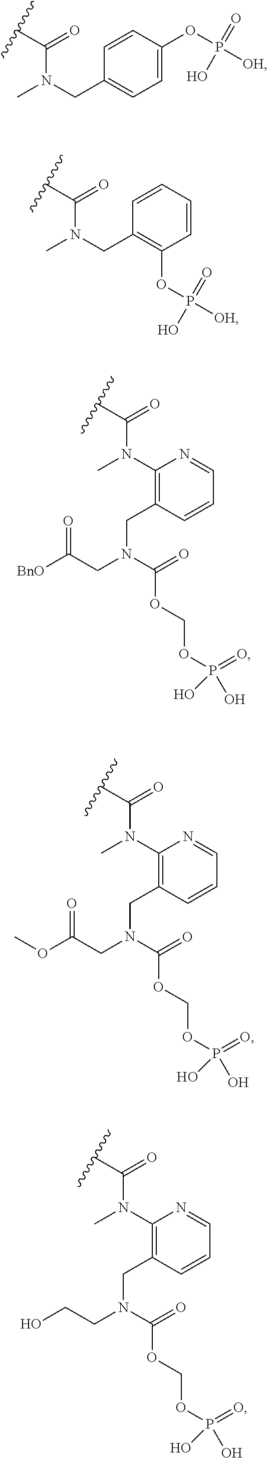 [SMILES: *C(=O)N(C)Cc1ccc(OP(=O)(O)O)cc1 ; *C(=O)N(C)Cc1ccccc1OP(=O)(O)O ; *C(=O)N(C)c1ncccc1CN(CC(=O)OCc1ccccc1)C(=O)OCOP(=O)(O)O ; *C(=O)N(C)c1ncccc1CN(CC(=O)OC)C(=O)OCOP(=O)(O)O ; *C(=O)N(C)c1ncccc1CN(CCO)C(=O)OCOP(=O)(O)O]

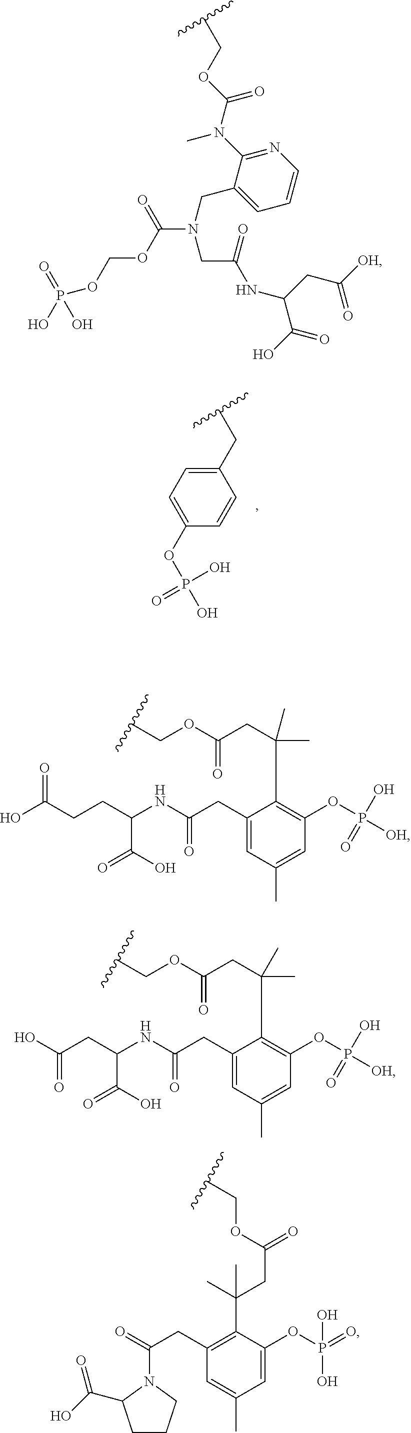

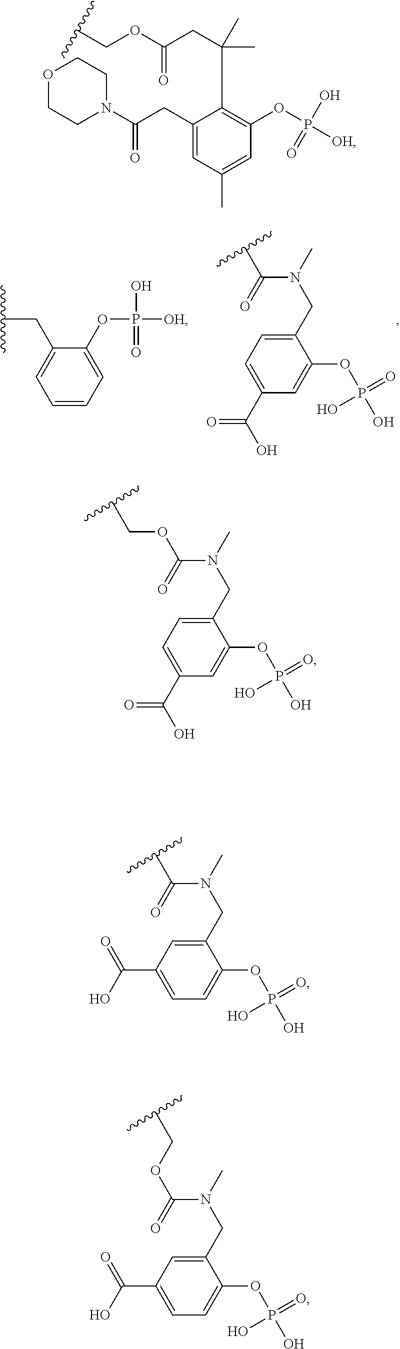 [SMILES: *COC(=O)CC(C)(C)c1c(CC(=O)N2CCOCC2)cc(C)cc1OP(=O)(O)O ; *Cc1ccccc1OP(=O)(O)O ; *C(=O)N(C)Cc1ccc(C(=O)O)cc1OP(=O)(O)O ; *COC(=O)N(C)Cc1ccc(C(=O)O)cc1OP(=O)(O)O ; *C(=O)N(C)Cc1cc(C(=O)O)ccc1OP(=O)(O)O ; *COC(=O)N(C)Cc1cc(C(=O)O)ccc1OP(=O)(O)O]

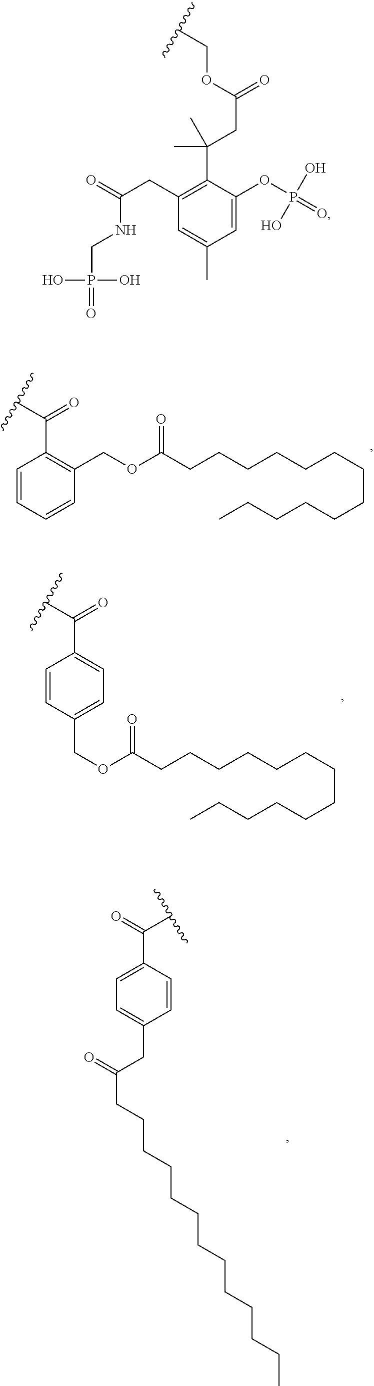 [SMILES: *COC(=O)CC(C)(C)c1c(CC(=O)NCP(=O)(O)O)cc(C)cc1OP(=O)(O)O ; *C(=O)c1ccccc1COC(=O)CCCCCCCCCCCCC ; *C(=O)c1ccc(COC(=O)CCCCCCCCCCCCC)cc1 ; *C(=O)c1ccc(CC(=O)CCCCCCCCCCCCC)cc1]

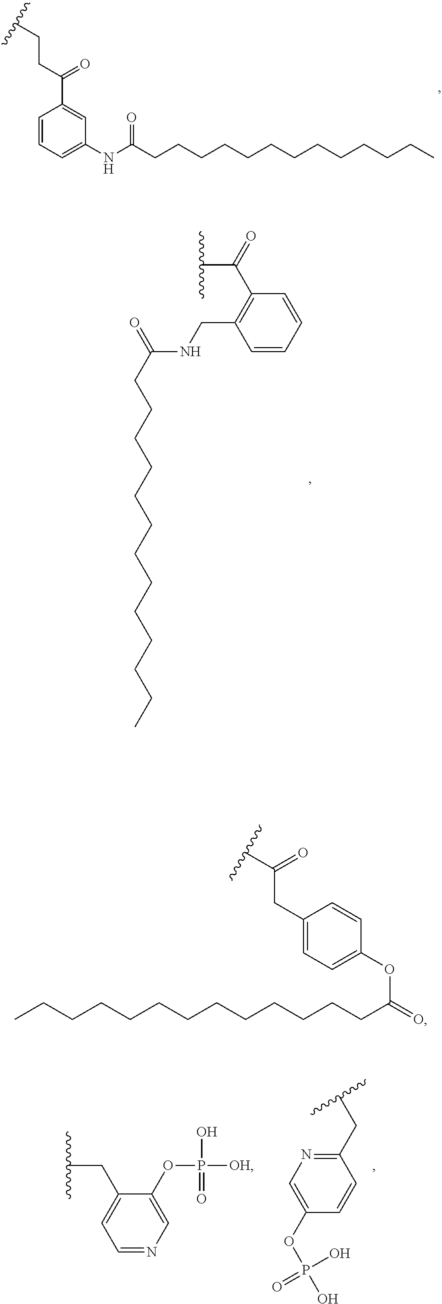

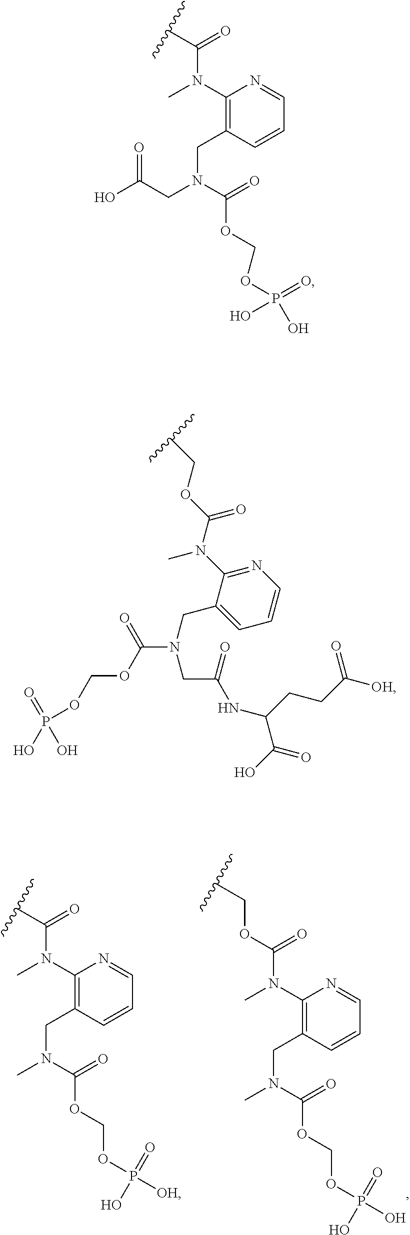 [SMILES: *C(=O)N(C)c1ncccc1CN(CC(=O)O)C(=O)OCOP(=O)(O)O ; *COC(=O)N(C)c1ncccc1CN(CC(=O)NC(CCC(=O)O)C(=O)O)C(=O)OCOP(=O)(O)O ; *C(=O)N(C)c1ncccc1CN(C)C(=O)OCOP(=O)(O)O ; *COC(=O)N(C)c1ncccc1CN(C)C(=O)OCOP(=O)(O)O]

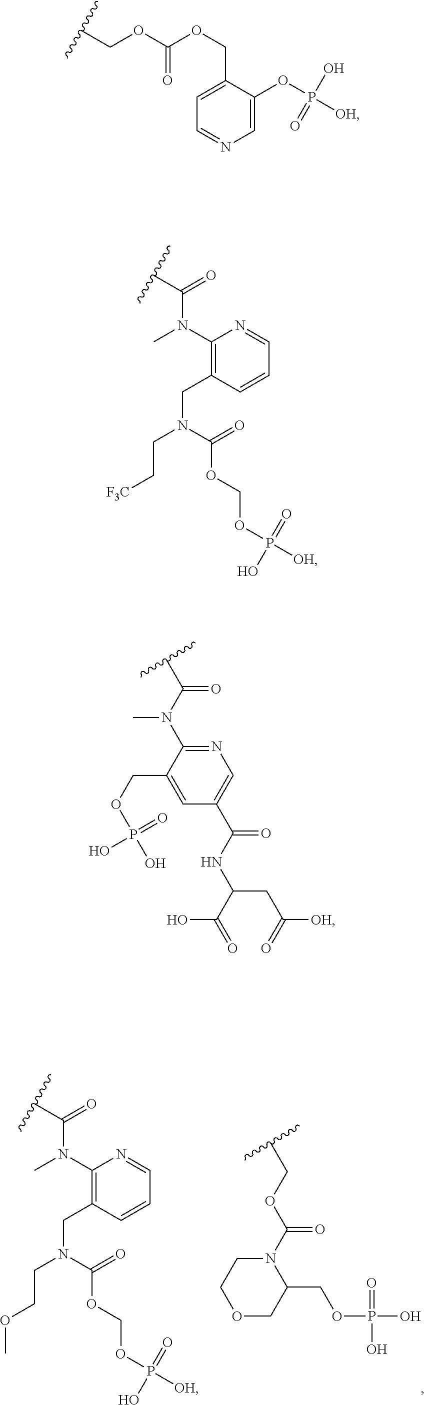 [SMILES: *COC(=O)OCc1ccncc1OP(=O)(O)O ; *C(=O)N(C)c1ncccc1CN(CCC(F)(F)F)C(=O)OCOP(=O)(O)O ; *C(=O)N(C)c1ncc(C(=O)NC(CC(=O)O)C(=O)O)cc1COP(=O)(O)O ; *C(=O)N(C)c1ncccc1CN(CCOC)C(=O)OCOP(=O)(O)O ; *COC(=O)N1CCOCC1COP(=O)(O)O]

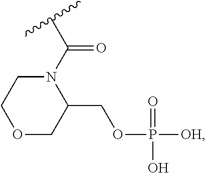 [SMILES: *C(=O)N1CCOCC1COP(=O)(O)O]

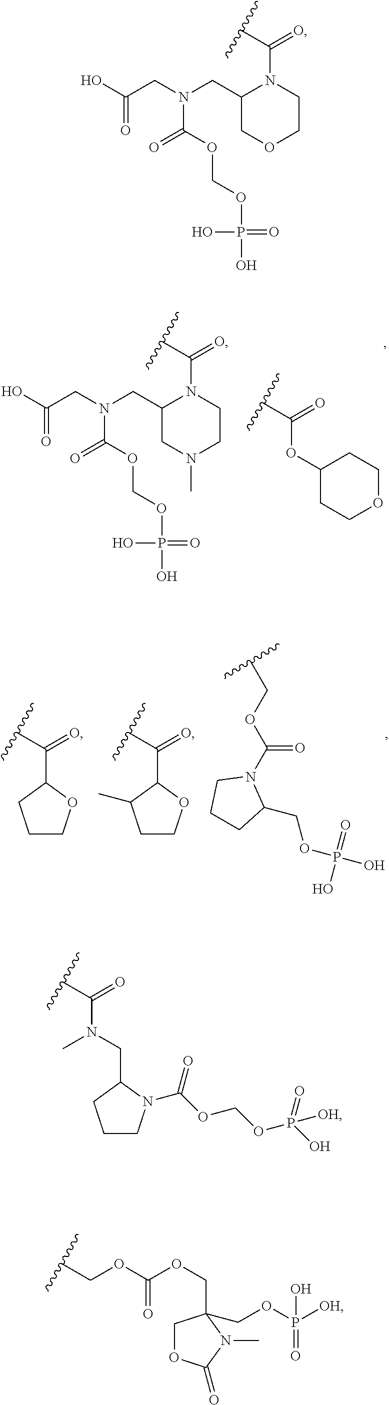

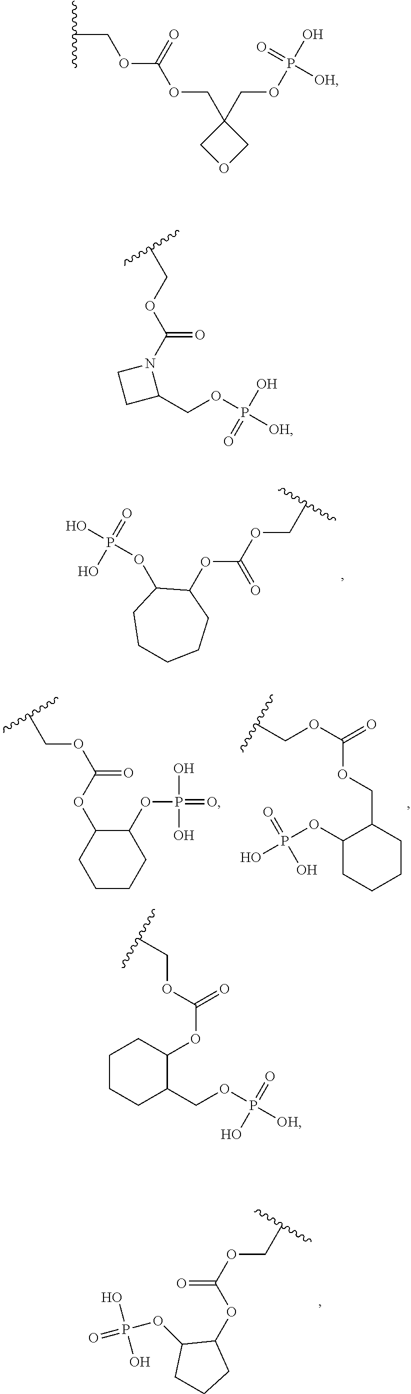

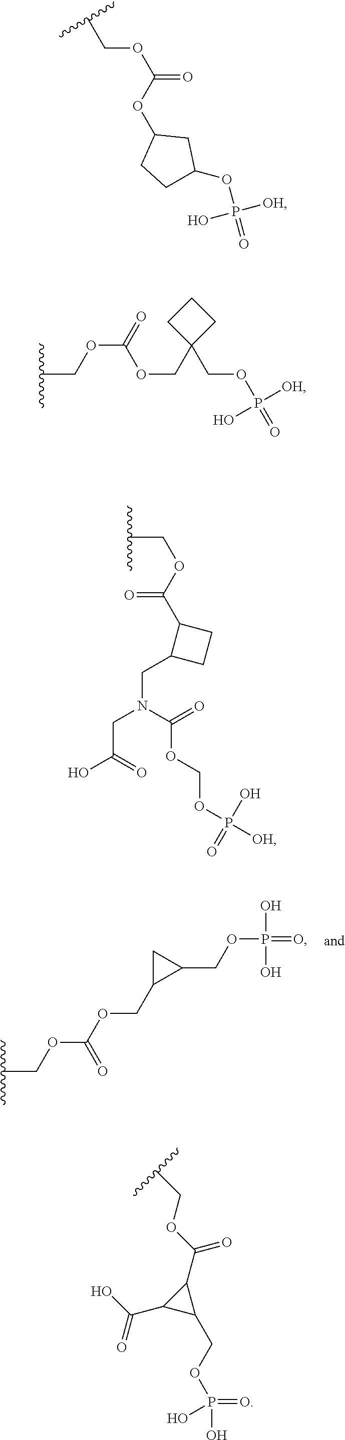 [SMILES: *COC(=O)OC1CCC(OP(=O)(O)O)C1 ; *COC(=O)OCC1(COP(=O)(O)O)CCC1 ; *COC(=O)C1CCC1CN(CC(=O)O)C(=O)OCOP(=O)(O)O ; *COC(=O)OCC1CC1COP(=O)(O)O ; *COC(=O)C1C(COP(=O)(O)O)C1C(=O)O]

In some embodiments, R is selected from the group consisting of:

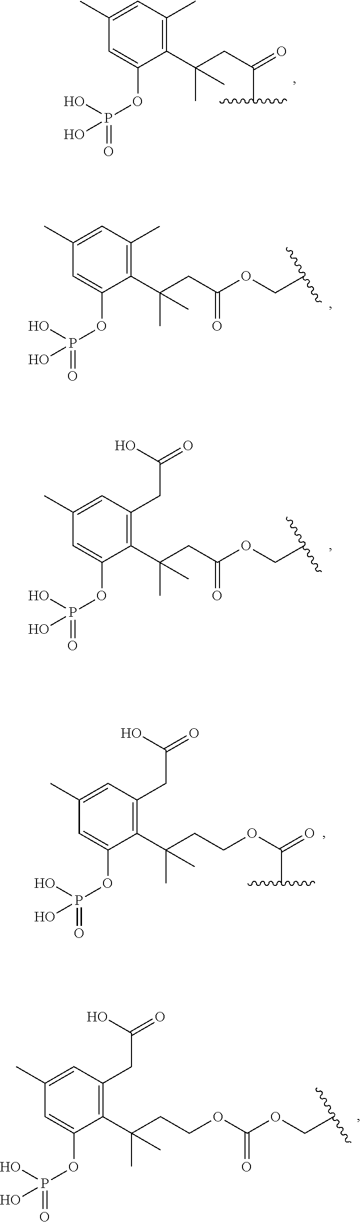 [SMILES: *C(=O)CC(C)(C)c1c(C)cc(C)cc1OP(=O)(O)O ; *COC(=O)CC(C)(C)c1c(C)cc(C)cc1OP(=O)(O)O ; *COC(=O)CC(C)(C)c1c(CC(=O)O)cc(C)cc1OP(=O)(O)O ; *C(=O)OCCC(C)(C)c1c(CC(=O)O)cc(C)cc1OP(=O)(O)O ; *COC(=O)OCCC(C)(C)c1c(CC(=O)O)cc(C)cc1OP(=O)(O)O]

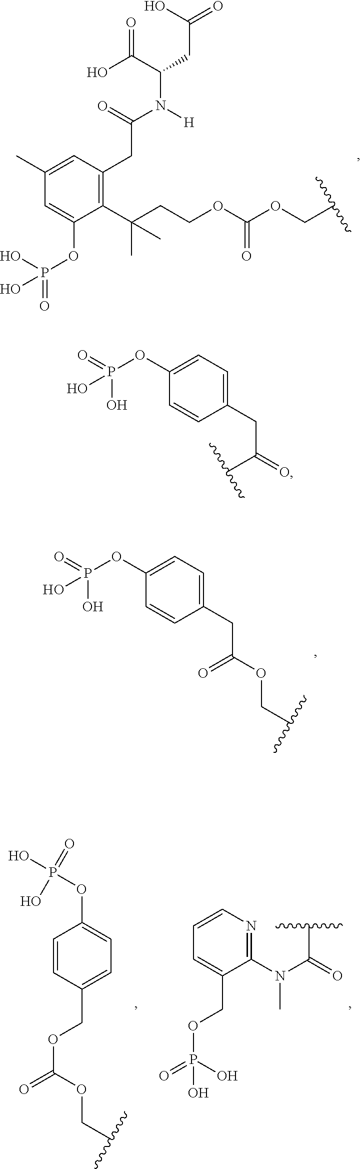 [SMILES: *COC(=O)OCCC(C)(C)c1c(CC(=O)N[C@@H](CC(=O)O)C(=O)O)cc(C)cc1OP(=O)(O)O ; *C(=O)Cc1ccc(OP(=O)(O)O)cc1 ; *COC(=O)Cc1ccc(OP(=O)(O)O)cc1 ; *COC(=O)OCc1ccc(OP(=O)(O)O)cc1 ; *C(=O)N(C)c1ncccc1COP(=O)(O)O]

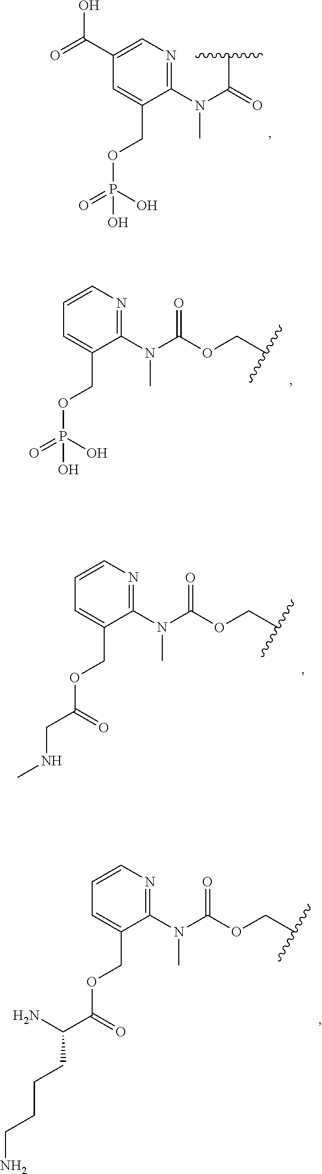 [SMILES: *C(=O)N(C)c1ncc(C(=O)O)cc1COP(=O)(O)O ; *COC(=O)N(C)c1ncccc1COP(=O)(O)O ; *COC(=O)N(C)c1ncccc1COC(=O)CNC ; *COC(=O)N(C)c1ncccc1COC(=O)[C@@H](N)CCCCN]

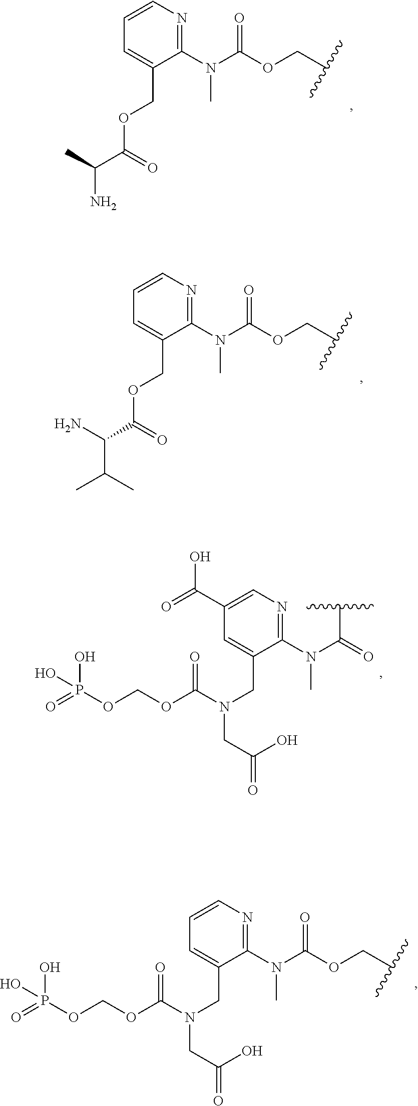 [SMILES: *COC(=O)N(C)c1ncccc1COC(=O)[C@H](C)N ; *COC(=O)N(C)c1ncccc1COC(=O)[C@@H](N)C(C)C ; *C(=O)N(C)c1ncc(C(=O)O)cc1CN(CC(=O)O)C(=O)OCOP(=O)(O)O ; *COC(=O)N(C)c1ncccc1CN(CC(=O)O)C(=O)OCOP(=O)(O)O]

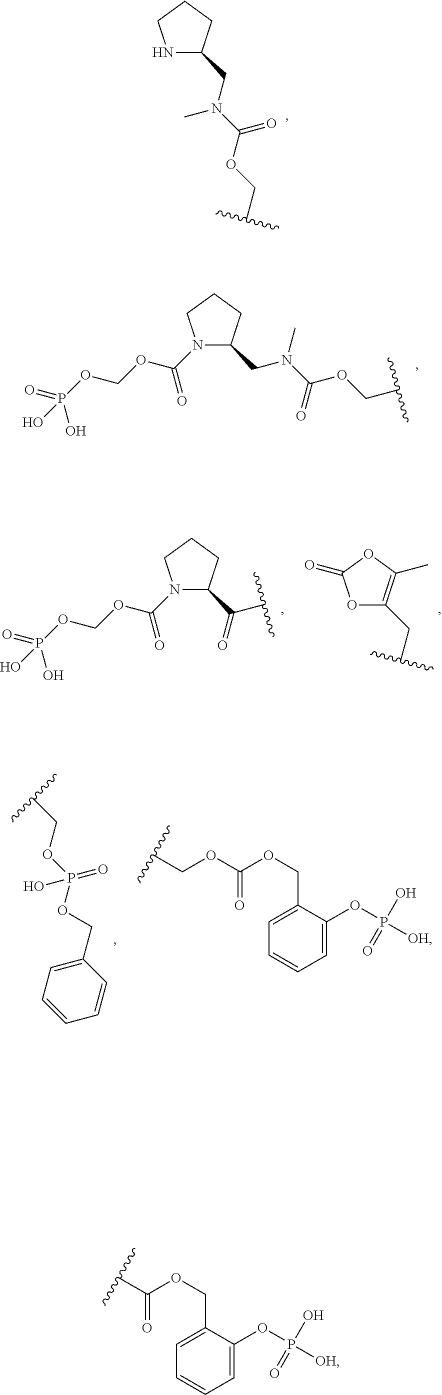

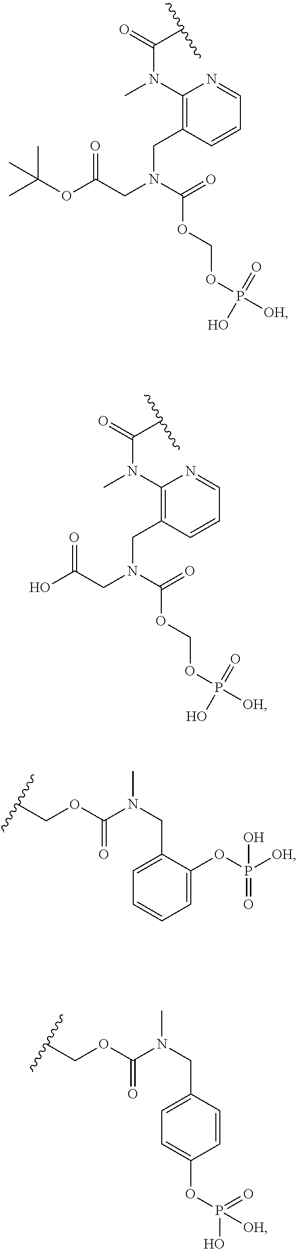 [SMILES: *C(=O)N(C)c1ncccc1CN(CC(=O)OC(C)(C)C)C(=O)OCOP(=O)(O)O ; *C(=O)N(C)c1ncccc1CN(CC(=O)O)C(=O)OCOP(=O)(O)O ; *COC(=O)N(C)Cc1ccccc1OP(=O)(O)O ; *COC(=O)N(C)Cc1ccc(OP(=O)(O)O)cc1]

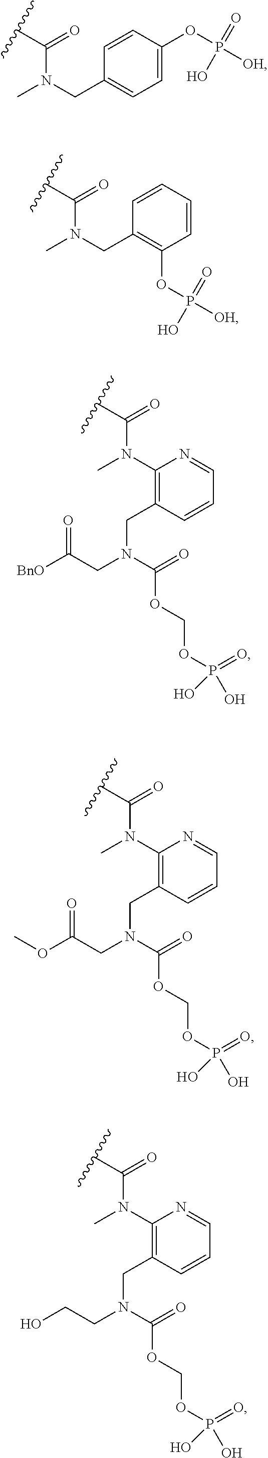 [SMILES: *C(=O)N(C)Cc1ccc(OP(=O)(O)O)cc1 ; *C(=O)N(C)Cc1ccccc1OP(=O)(O)O ; *C(=O)N(C)c1ncccc1CN(CC(=O)OCc1ccccc1)C(=O)OCOP(=O)(O)O ; *C(=O)N(C)c1ncccc1CN(CC(=O)OC)C(=O)OCOP(=O)(O)O ; *C(=O)N(C)c1ncccc1CN(CCO)C(=O)OCOP(=O)(O)O]

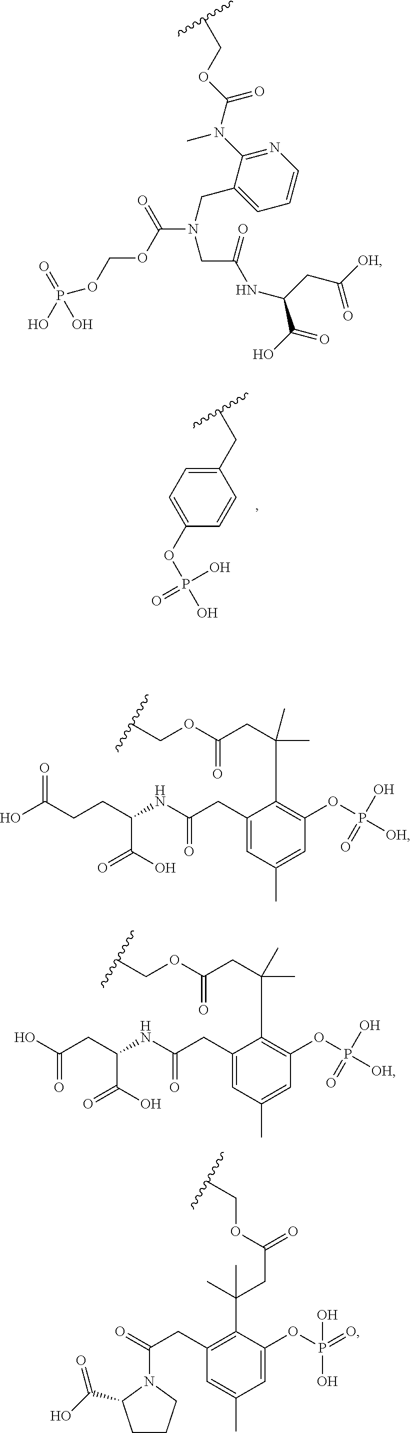

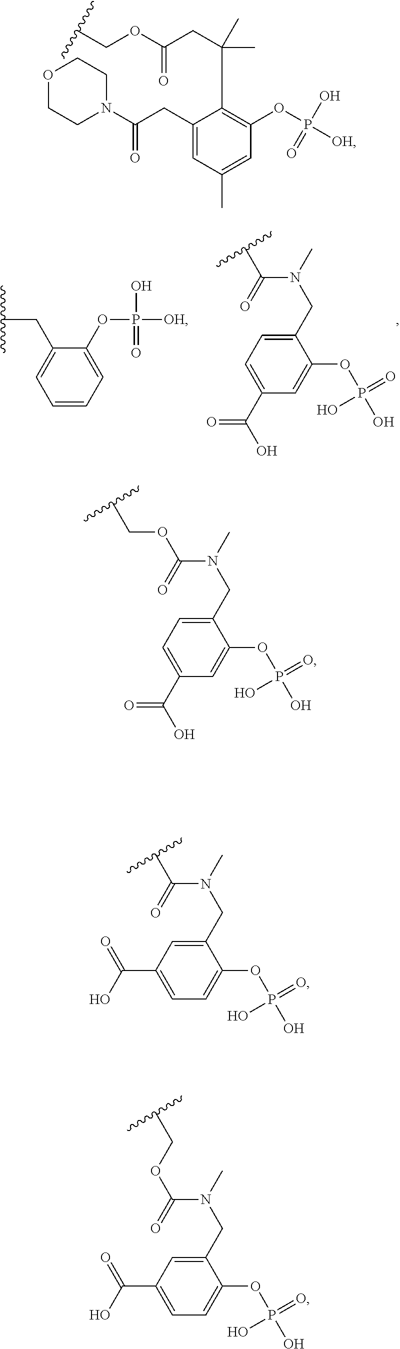 [SMILES: *COC(=O)CC(C)(C)c1c(CC(=O)N2CCOCC2)cc(C)cc1OP(=O)(O)O ; *Cc1ccccc1OP(=O)(O)O ; *C(=O)N(C)Cc1ccc(C(=O)O)cc1OP(=O)(O)O ; *COC(=O)N(C)Cc1ccc(C(=O)O)cc1OP(=O)(O)O ; *C(=O)N(C)Cc1cc(C(=O)O)ccc1OP(=O)(O)O ; *COC(=O)N(C)Cc1cc(C(=O)O)ccc1OP(=O)(O)O]

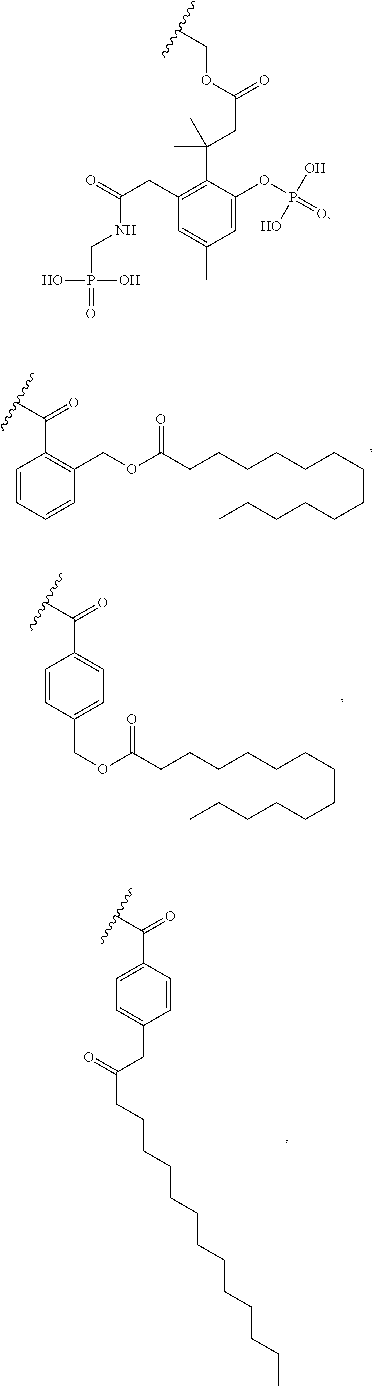 [SMILES: *COC(=O)CC(C)(C)c1c(CC(=O)NCP(=O)(O)O)cc(C)cc1OP(=O)(O)O ; *C(=O)c1ccccc1COC(=O)CCCCCCCCCCCCC ; *C(=O)c1ccc(COC(=O)CCCCCCCCCCCCC)cc1 ; *C(=O)c1ccc(CC(=O)CCCCCCCCCCCCC)cc1]

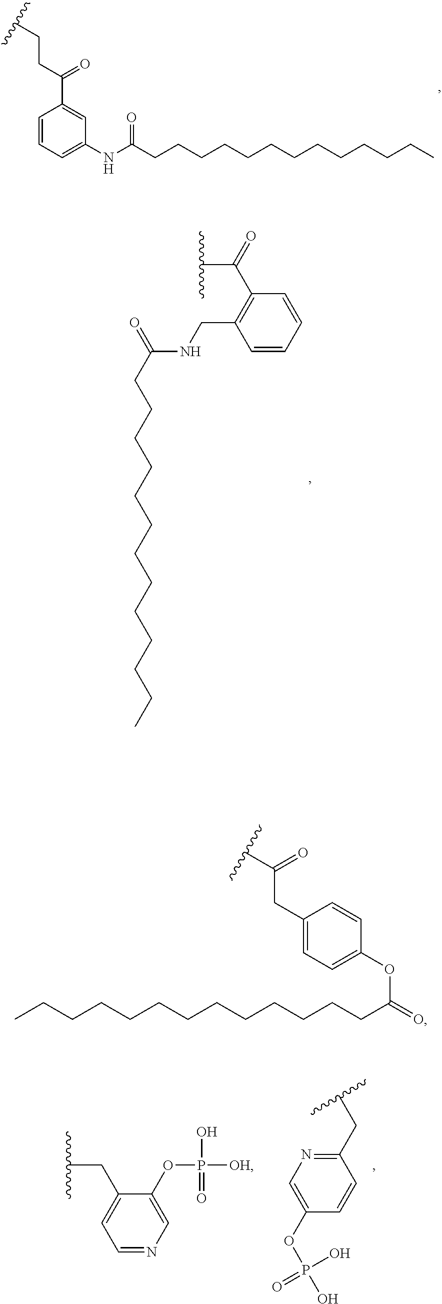

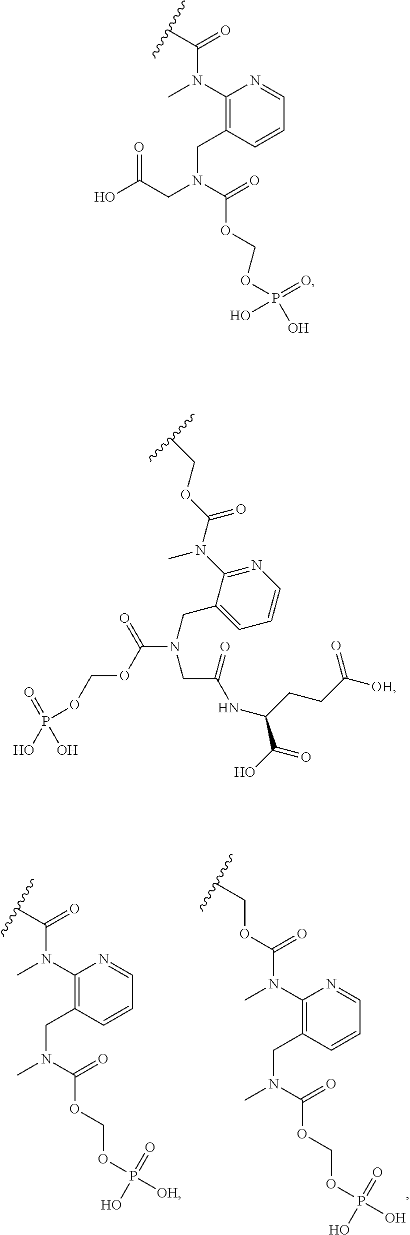 [SMILES: *C(=O)N(C)c1ncccc1CN(CC(=O)O)C(=O)OCOP(=O)(O)O ; *COC(=O)N(C)c1ncccc1CN(CC(=O)N[C@@H](CCC(=O)O)C(=O)O)C(=O)OCOP(=O)(O)O ; *C(=O)N(C)c1ncccc1CN(C)C(=O)OCOP(=O)(O)O ; *COC(=O)N(C)c1ncccc1CN(C)C(=O)OCOP(=O)(O)O]

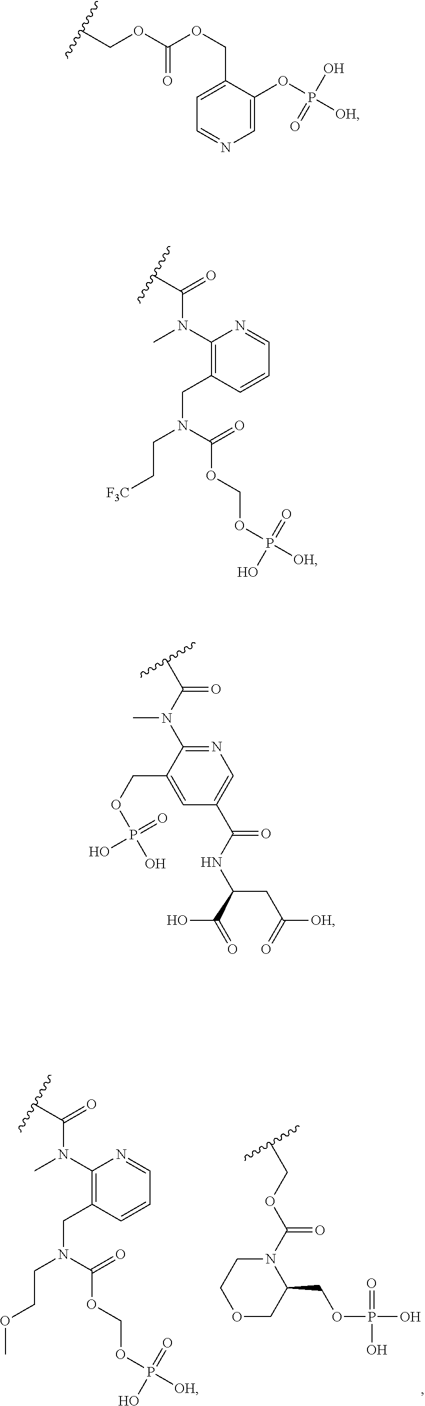 [SMILES: *COC(=O)OCc1ccncc1OP(=O)(O)O ; *C(=O)N(C)c1ncccc1CN(CCC(F)(F)F)C(=O)OCOP(=O)(O)O ; *C(=O)N(C)c1ncc(C(=O)N[C@@H](CC(=O)O)C(=O)O)cc1COP(=O)(O)O ; *C(=O)N(C)c1ncccc1CN(CCOC)C(=O)OCOP(=O)(O)O ; *COC(=O)N1CCOC[C@@H]1COP(=O)(O)O]

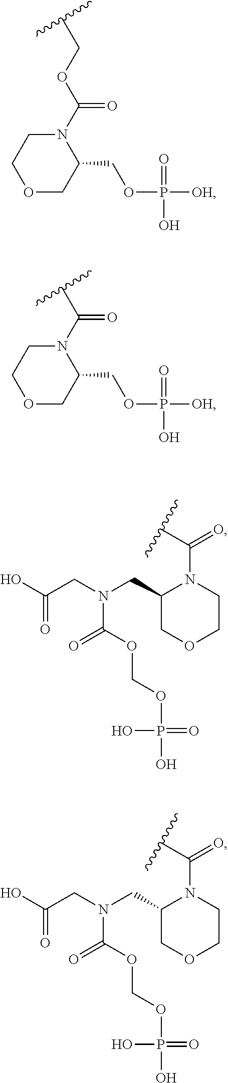 [SMILES: *COC(=O)N1CCOC[C@H]1COP(=O)(O)O ; *C(=O)N1CCOC[C@H]1COP(=O)(O)O ; *C(=O)N1CCOC[C@H]1CN(CC(=O)O)C(=O)OCOP(=O)(O)O ; *C(=O)N1CCOC[C@@H]1CN(CC(=O)O)C(=O)OCOP(=O)(O)O]

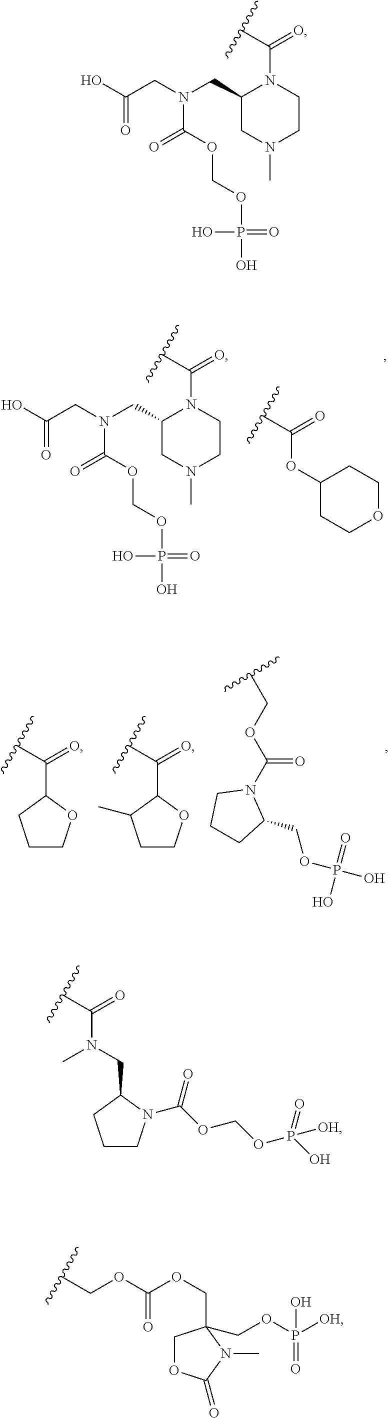

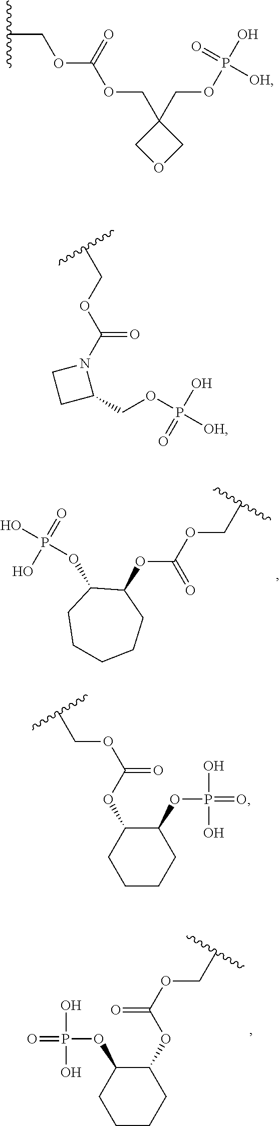 [SMILES: *COC(=O)OCC1(COP(=O)(O)O)COC1 ; *COC(=O)N1CC[C@H]1COP(=O)(O)O ; *COC(=O)O[C@H]1CCCCC[C@@H]1OP(=O)(O)O ; *COC(=O)O[C@H]1CCCC[C@@H]1OP(=O)(O)O ; *COC(=O)O[C@@H]1CCCC[C@H]1OP(=O)(O)O]

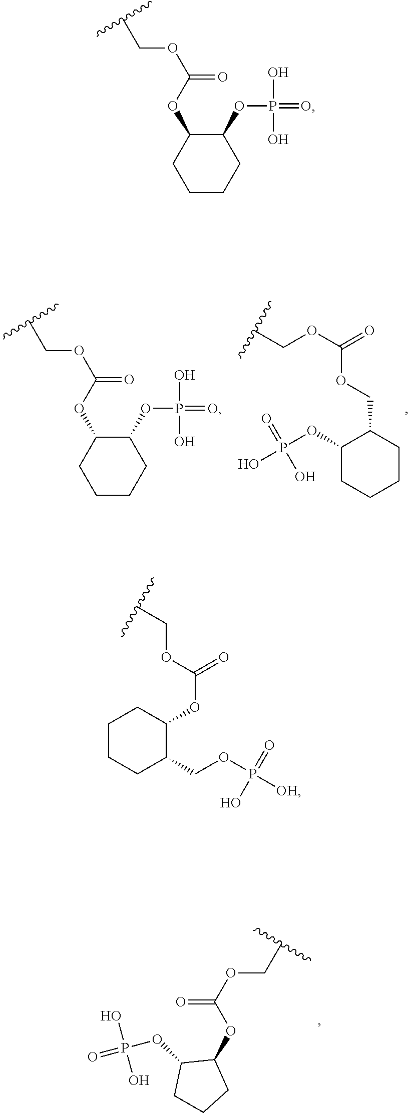 [SMILES: *COC(=O)O[C@@H]1CCCC[C@@H]1OP(=O)(O)O ; *COC(=O)O[C@H]1CCCC[C@H]1OP(=O)(O)O ; *COC(=O)OC[C@@H]1CCCC[C@@H]1OP(=O)(O)O ; *COC(=O)O[C@H]1CCCC[C@H]1COP(=O)(O)O ; *COC(=O)O[C@H]1CCC[C@@H]1OP(=O)(O)O]

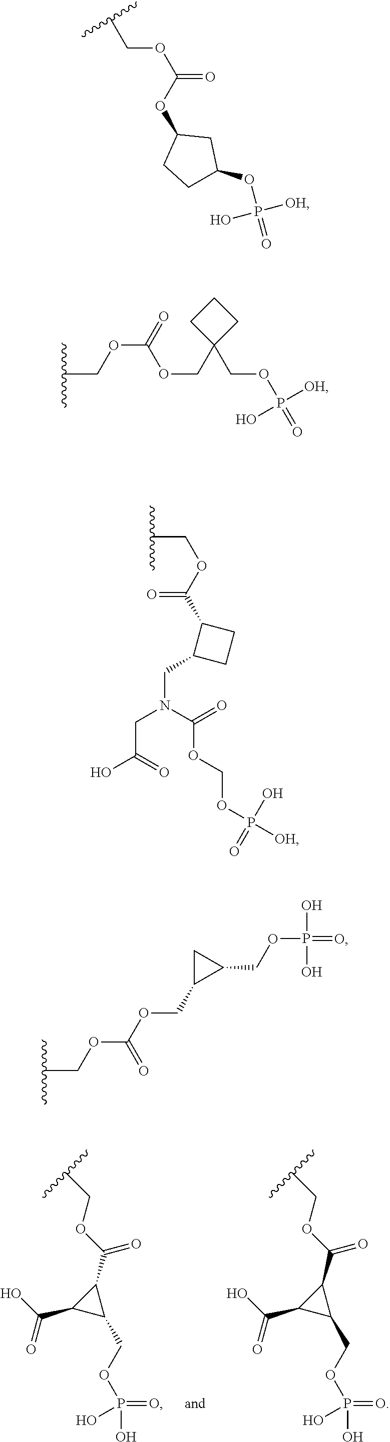 [SMILES: *COC(=O)O[C@@H]1CC[C@H](OP(=O)(O)O)C1 ; *COC(=O)OCC1(COP(=O)(O)O)CCC1 ; *COC(=O)[C@@H]1CC[C@@H]1CN(CC(=O)O)C(=O)OCOP(=O)(O)O ; *COC(=O)OC[C@@H]1C[C@@H]1COP(=O)(O)O ; *COC(=O)[C@@H]1[C@H](COP(=O)(O)O)[C@H]1C(=O)O ; *COC(=O)[C@H]1[C@@H](COP(=O)(O)O)[C@H]1C(=O)O]

In some embodiments, R¹ is selected from the group consisting of:

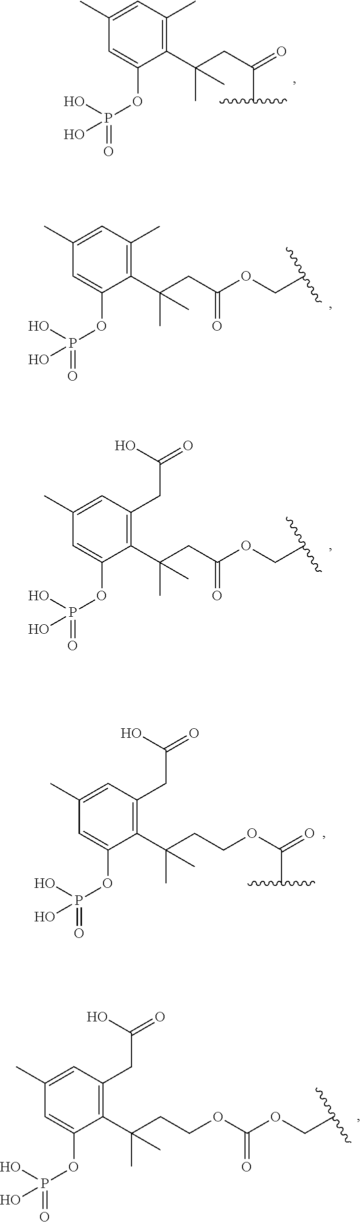 [SMILES: *C(=O)CC(C)(C)c1c(C)cc(C)cc1OP(=O)(O)O ; *COC(=O)CC(C)(C)c1c(C)cc(C)cc1OP(=O)(O)O ; *COC(=O)CC(C)(C)c1c(CC(=O)O)cc(C)cc1OP(=O)(O)O ; *C(=O)OCCC(C)(C)c1c(CC(=O)O)cc(C)cc1OP(=O)(O)O ; *COC(=O)OCCC(C)(C)c1c(CC(=O)O)cc(C)cc1OP(=O)(O)O]

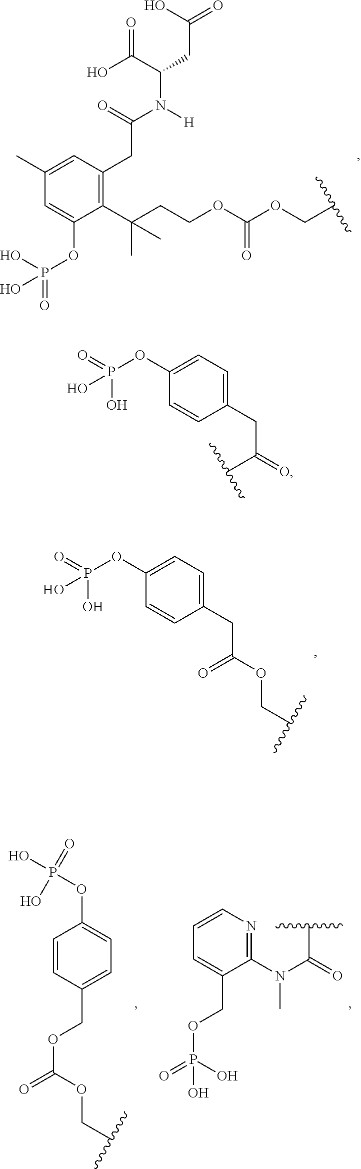 [SMILES: *COC(=O)OCCC(C)(C)c1c(CC(=O)N[C@@H](CC(=O)O)C(=O)O)cc(C)cc1OP(=O)(O)O ; *C(=O)Cc1ccc(OP(=O)(O)O)cc1 ; *COC(=O)Cc1ccc(OP(=O)(O)O)cc1 ; *COC(=O)OCc1ccc(OP(=O)(O)O)cc1 ; *C(=O)N(C)c1ncccc1COP(=O)(O)O]

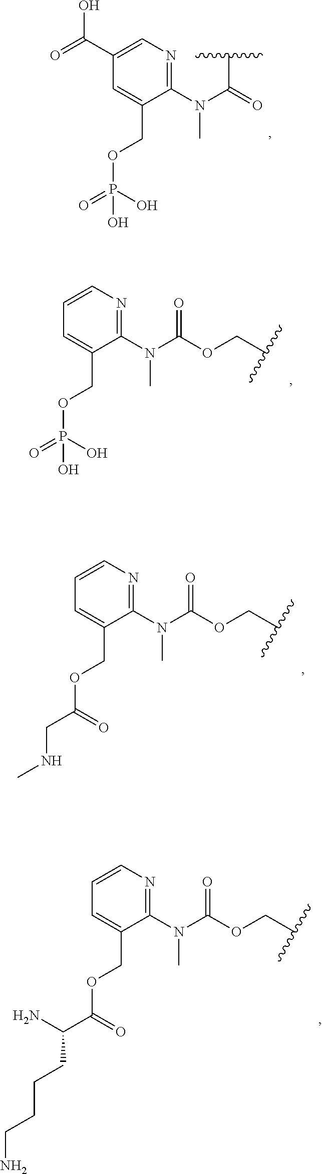 [SMILES: *C(=O)N(C)c1ncc(C(=O)O)cc1COP(=O)(O)O ; *COC(=O)N(C)c1ncccc1COP(=O)(O)O ; *COC(=O)N(C)c1ncccc1COC(=O)CNC ; *COC(=O)N(C)c1ncccc1COC(=O)[C@@H](N)CCCCN]

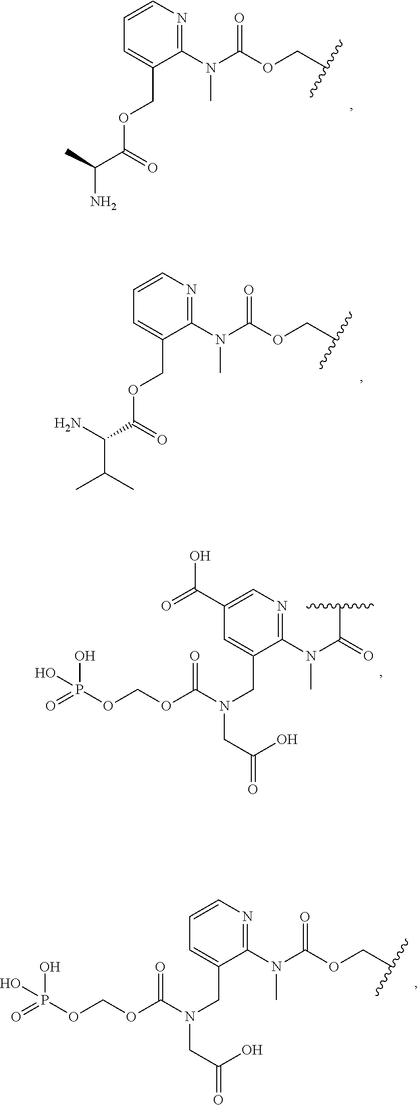 [SMILES: *COC(=O)N(C)c1ncccc1COC(=O)[C@H](C)N ; *COC(=O)N(C)c1ncccc1COC(=O)[C@@H](N)C(C)C ; *C(=O)N(C)c1ncc(C(=O)O)cc1CN(CC(=O)O)C(=O)OCOP(=O)(O)O ; *COC(=O)N(C)c1ncccc1CN(CC(=O)O)C(=O)OCOP(=O)(O)O]

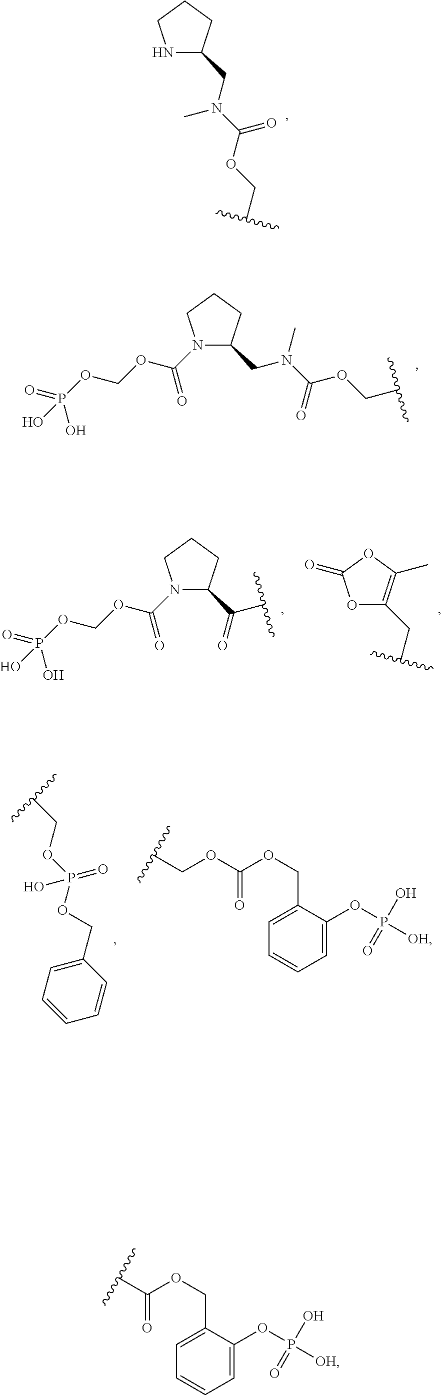

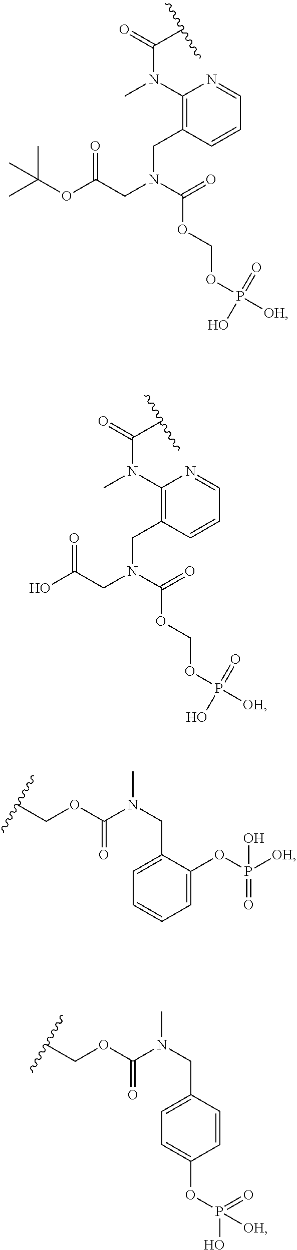 [SMILES: *C(=O)N(C)c1ncccc1CN(CC(=O)OC(C)(C)C)C(=O)OCOP(=O)(O)O ; *C(=O)N(C)c1ncccc1CN(CC(=O)O)C(=O)OCOP(=O)(O)O ; *COC(=O)N(C)Cc1ccccc1OP(=O)(O)O ; *COC(=O)N(C)Cc1ccc(OP(=O)(O)O)cc1]

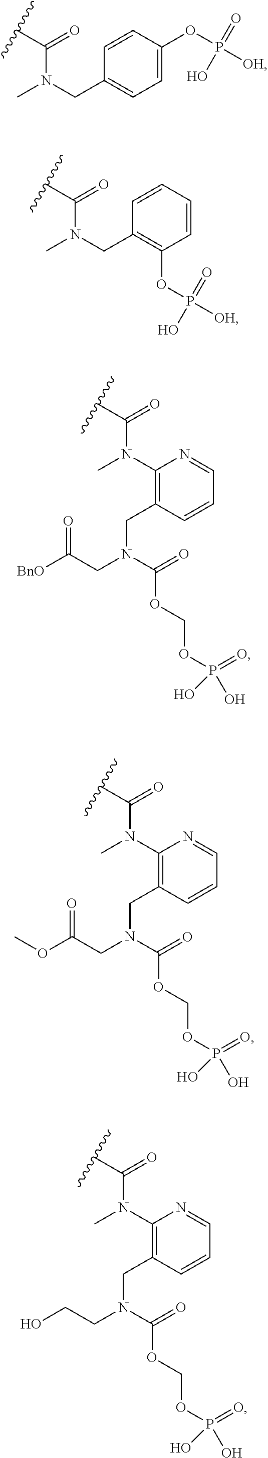 [SMILES: *C(=O)N(C)Cc1ccc(OP(=O)(O)O)cc1 ; *C(=O)N(C)Cc1ccccc1OP(=O)(O)O ; *C(=O)N(C)c1ncccc1CN(CC(=O)OCc1ccccc1)C(=O)OCOP(=O)(O)O ; *C(=O)N(C)c1ncccc1CN(CC(=O)OC)C(=O)OCOP(=O)(O)O ; *C(=O)N(C)c1ncccc1CN(CCO)C(=O)OCOP(=O)(O)O]

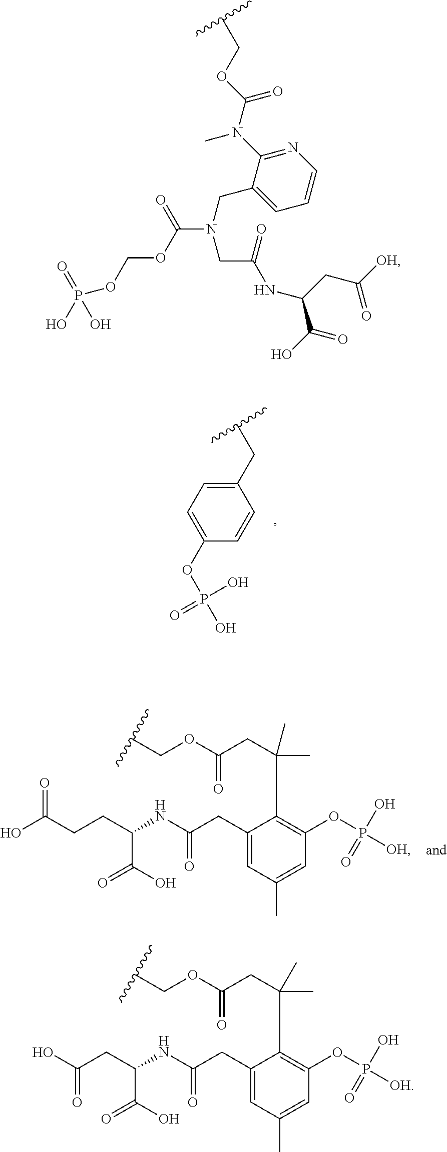 [SMILES: *COC(=O)N(C)c1ncccc1CN(CC(=O)N[C@@H](CC(=O)O)C(=O)O)C(=O)OCOP(=O)(O)O ; *Cc1ccc(OP(=O)(O)O)cc1 ; *COC(=O)CC(C)(C)c1c(CC(=O)N[C@@H](CCC(=O)O)C(=O)O)cc(C)cc1OP(=O)(O)O ; *COC(=O)CC(C)(C)c1c(CC(=O)N[C@@H](CC(=O)O)C(=O)O)cc(C)cc1OP(=O)(O)O]

In some embodiments, R² is methyl or methoxy. In some embodiments, R² is methyl. In some embodiments, R² is methoxy.

In some embodiments, R³ and R⁶ are each independently a halo. In some embodiments, R³ and R⁶ are each F.

In some embodiments, R⁴, R⁵ and R⁷ are each H.

In some embodiments, R⁸ is C₁₋₃alkyl. In some embodiments, R⁸ is methyl.

In some embodiments of the compound of Formula I, the compound is selected from the group consisting of:

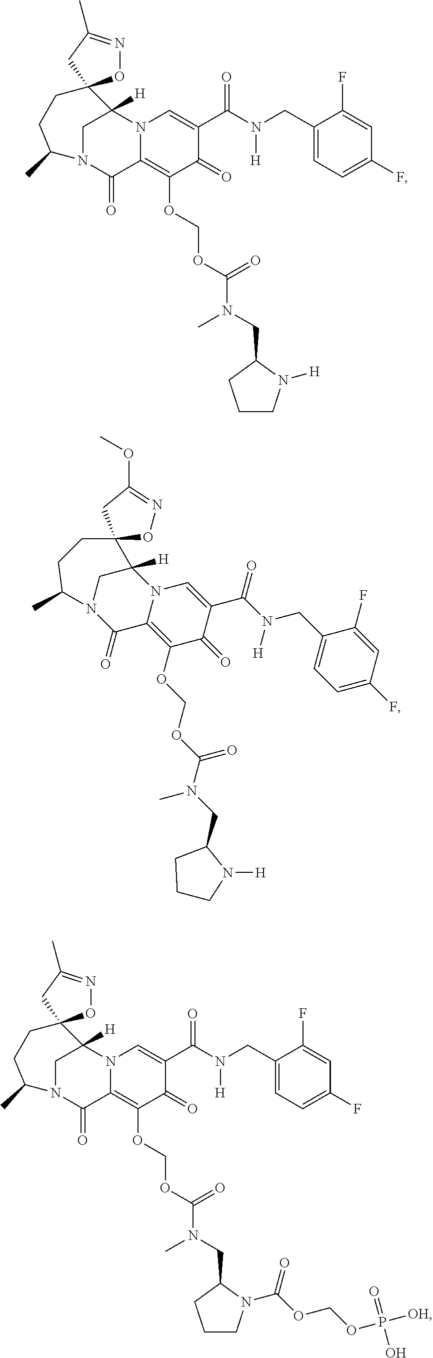 [SMILES: CC1=NO[C@@]2(CC[C@H](C)N3C[C@H]2n2cc(C(=O)NCc4ccc(F)cc4F)c(=O)c(OCOC(=O)N(C)C[C@@H]4CCCN4)c2C3=O)C1 ; COC1=NO[C@@]2(CC[C@H](C)N3C[C@H]2n2cc(C(=O)NCc4ccc(F)cc4F)c(=O)c(OCOC(=O)N(C)C[C@@H]4CCCN4)c2C3=O)C1 ; CC1=NO[C@@]2(CC[C@H](C)N3C[C@H]2n2cc(C(=O)NCc4ccc(F)cc4F)c(=O)c(OCOC(=O)N(C)C[C@@H]4CCCN4C(=O)OCOP(=O)(O)O)c2C3=O)C1]

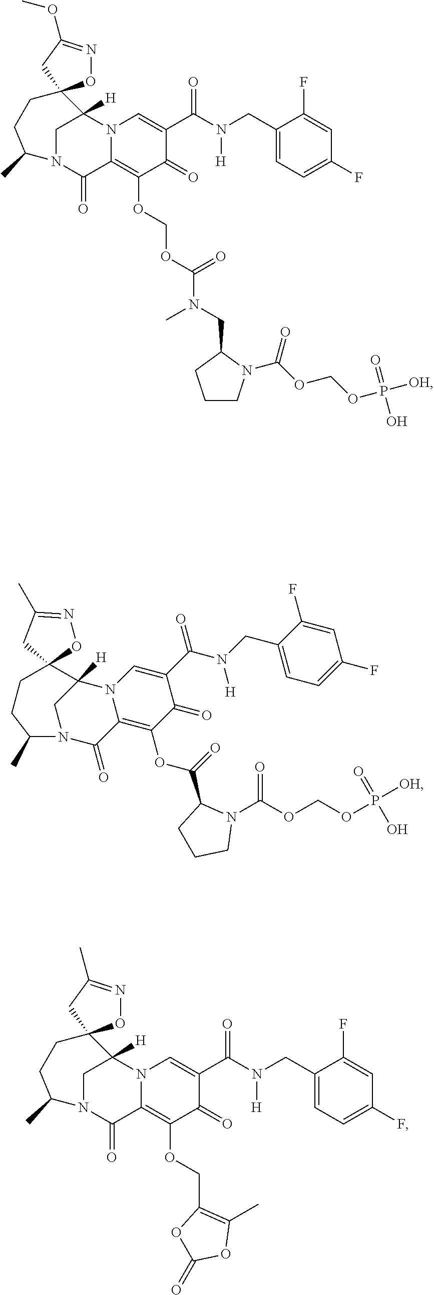 [SMILES: COC1=NO[C@@]2(CC[C@H](C)N3C[C@H]2n2cc(C(=O)NCc4ccc(F)cc4F)c(=O)c(OCOC(=O)N(C)C[C@@H]4CCCN4C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)[C@@H]4CCCN4C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCc4oc(=O)oc4C)c2C3=O)C1]

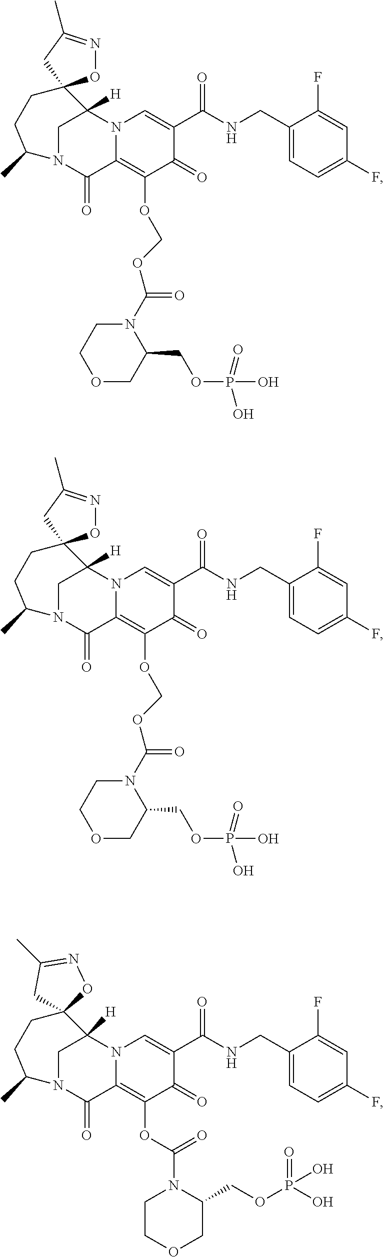 [SMILES: CC1=NO[C@@]2(CC[C@H](C)N3C[C@H]2n2cc(C(=O)NCc4ccc(F)cc4F)c(=O)c(OCOC(=O)N4CCOC[C@@H]4COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)N4CCOC[C@H]4COP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N4CCOC[C@H]4COP(=O)(O)O)c2C3=O)C1]

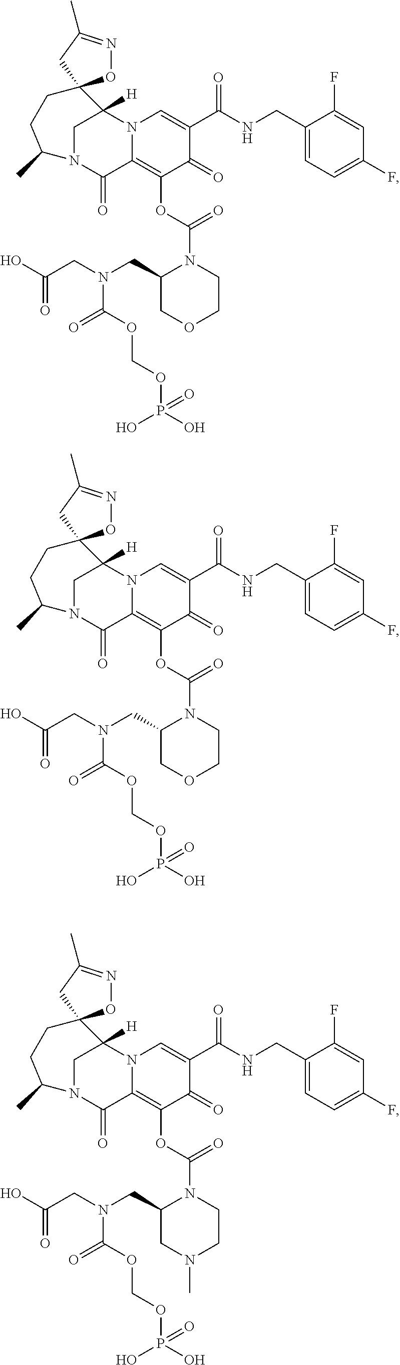 [SMILES: CC1=NO[C@@]2(CC[C@H](C)N3C[C@H]2n2cc(C(=O)NCc4ccc(F)cc4F)c(=O)c(OC(=O)N4CCOC[C@H]4CN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N4CCOC[C@@H]4CN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N4CCN(C)C[C@H]4CN(CC(=O)O)C(=O)OCOP(=O)(O)O)c2C3=O)C1]

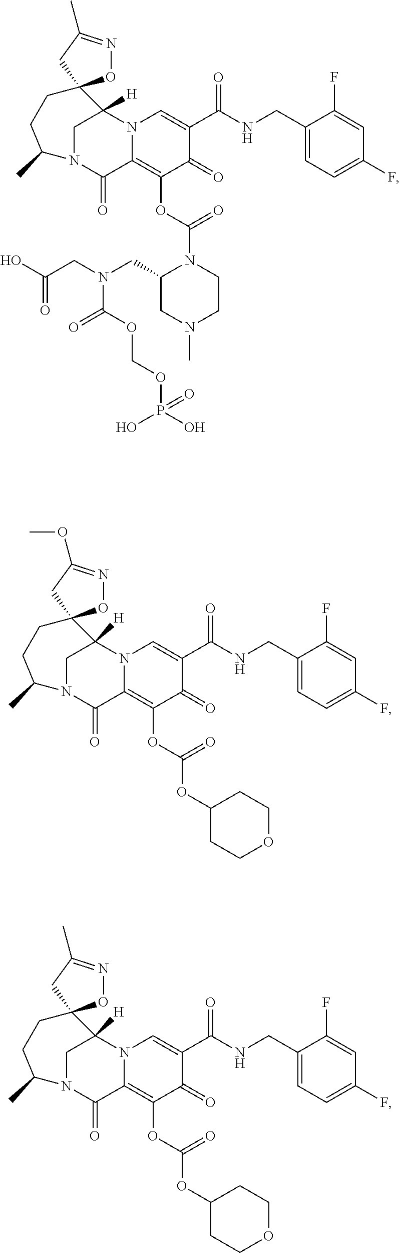 [SMILES: CC1=NO[C@@]2(CC[C@H](C)N3C[C@H]2n2cc(C(=O)NCc4ccc(F)cc4F)c(=O)c(OC(=O)N4CCN(C)C[C@@H]4CN(CC(=O)O)C(=O)OCOP(=O)(O)O)c2C3=O)C1 ; COC1=NO[C@@]2(CC[C@H](C)N3C[C@H]2n2cc(C(=O)NCc4ccc(F)cc4F)c(=O)c(OC(=O)OC4CCOCC4)c2C3=O)C1 ; CC1=NO[C@@]2(CC[C@H](C)N3C[C@H]2n2cc(C(=O)NCc4ccc(F)cc4F)c(=O)c(OC(=O)OC4CCOCC4)c2C3=O)C1]

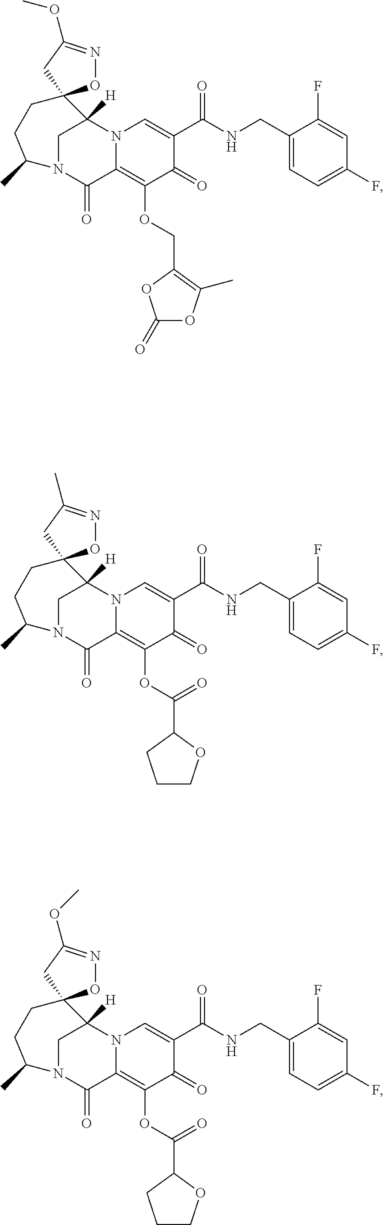 [SMILES: COC1=NO[C@@]2(CC[C@H](C)N3C[C@H]2n2cc(C(=O)NCc4ccc(F)cc4F)c(=O)c(OCc4oc(=O)oc4C)c2C3=O)C1 ; CC1=NO[C@@]2(CC[C@H](C)N3C[C@H]2n2cc(C(=O)NCc4ccc(F)cc4F)c(=O)c(OC(=O)C4CCCO4)c2C3=O)C1 ; COC1=NO[C@@]2(CC[C@H](C)N3C[C@H]2n2cc(C(=O)NCc4ccc(F)cc4F)c(=O)c(OC(=O)C4CCCO4)c2C3=O)C1]

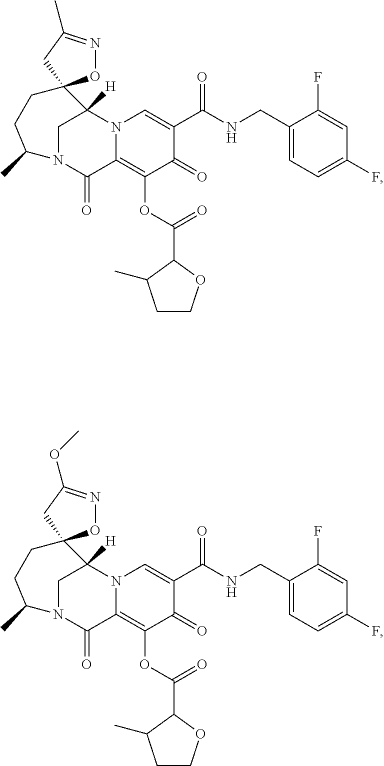 [SMILES: CC1=NO[C@@]2(CC[C@H](C)N3C[C@H]2n2cc(C(=O)NCc4ccc(F)cc4F)c(=O)c(OC(=O)C4OCCC4C)c2C3=O)C1 ; COC1=NO[C@@]2(CC[C@H](C)N3C[C@H]2n2cc(C(=O)NCc4ccc(F)cc4F)c(=O)c(OC(=O)C4OCCC4C)c2C3=O)C1]

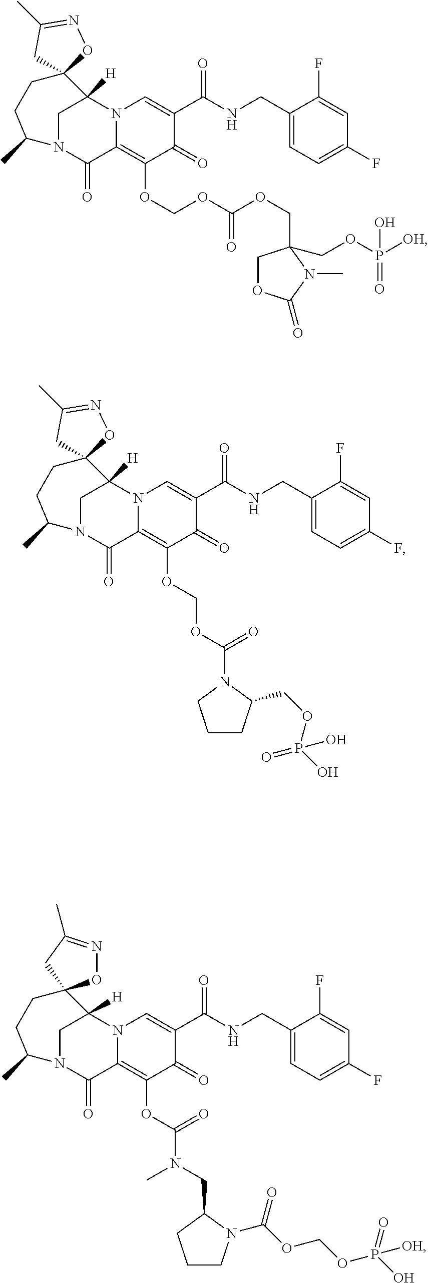 [SMILES: CC1=NO[C@@]2(CC[C@H](C)N3C[C@H]2n2cc(C(=O)NCc4ccc(F)cc4F)c(=O)c(OCOC(=O)OCC4(COP(=O)(O)O)COC(=O)N4C)c2C3=O)C1 ; CC1=NO[C@@]2(CC[C@H](C)N3C[C@H]2n2cc(C(=O)NCc4ccc(F)cc4F)c(=O)c(OCOC(=O)N4CCC[C@H]4COP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(C)C[C@@H]4CCCN4C(=O)OCOP(=O)(O)O)c2C3=O)C1]

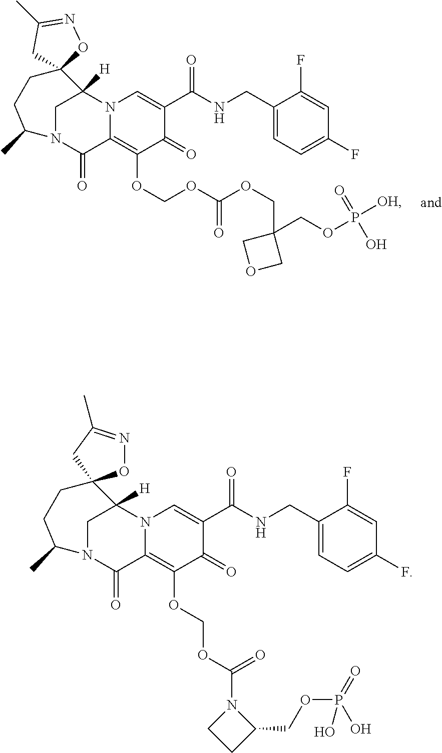 [SMILES: CC1=NO[C@@]2(CC[C@H](C)N3C[C@H]2n2cc(C(=O)NCc4ccc(F)cc4F)c(=O)c(OCOC(=O)OCC4(COP(=O)(O)O)COC4)c2C3=O)C1 ; CC1=NO[C@@]2(CC[C@H](C)N3C[C@H]2n2cc(C(=O)NCc4ccc(F)cc4F)c(=O)c(OCOC(=O)N4CC[C@H]4COP(=O)(O)O)c2C3=O)C1]

In some embodiments of the compound of Formula I, the compound is selected from the group consisting of:

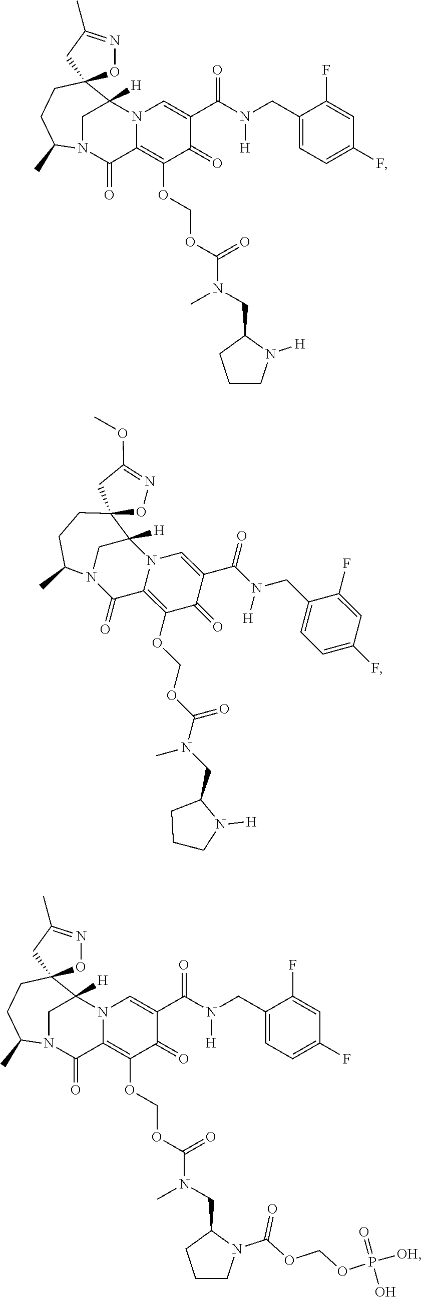 [SMILES: CC1=NO[C@@]2(CC[C@H](C)N3C[C@H]2n2cc(C(=O)NCc4ccc(F)cc4F)c(=O)c(OCOC(=O)N(C)C[C@@H]4CCCN4)c2C3=O)C1 ; COC1=NO[C@@]2(CC[C@H](C)N3C[C@H]2n2cc(C(=O)NCc4ccc(F)cc4F)c(=O)c(OCOC(=O)N(C)C[C@@H]4CCCN4)c2C3=O)C1 ; CC1=NO[C@@]2(CC[C@H](C)N3C[C@H]2n2cc(C(=O)NCc4ccc(F)cc4F)c(=O)c(OCOC(=O)N(C)C[C@@H]4CCCN4C(=O)OCOP(=O)(O)O)c2C3=O)C1]

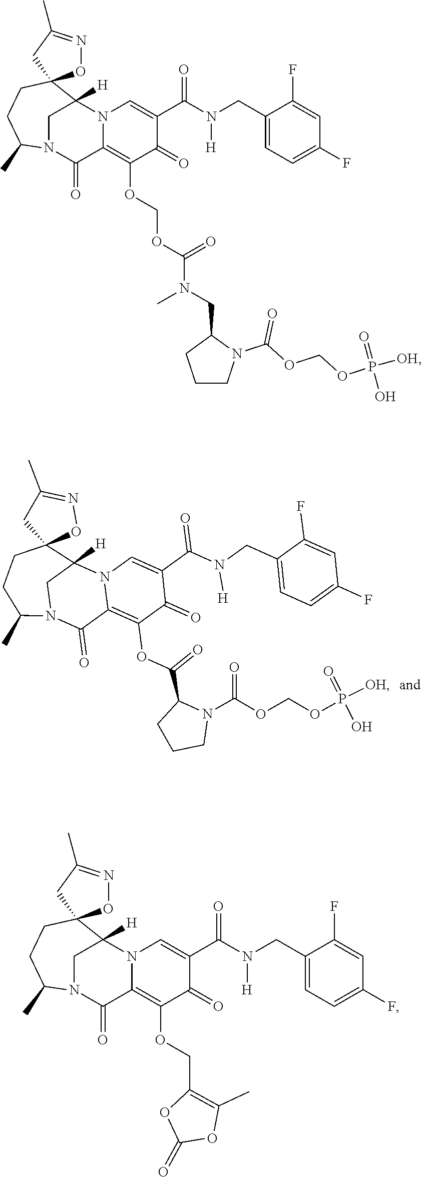 [SMILES: CC1=NO[C@@]2(CC[C@H](C)N3C[C@H]2n2cc(C(=O)NCc4ccc(F)cc4F)c(=O)c(OCOC(=O)N(C)C[C@@H]4CCCN4C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)[C@@H]4CCCN4C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCc4oc(=O)oc4C)c2C3=O)C1]

or a pharmaceutically acceptable salt thereof.

In some embodiments of the compound of Formula I, the compound is selected from the group consisting of:

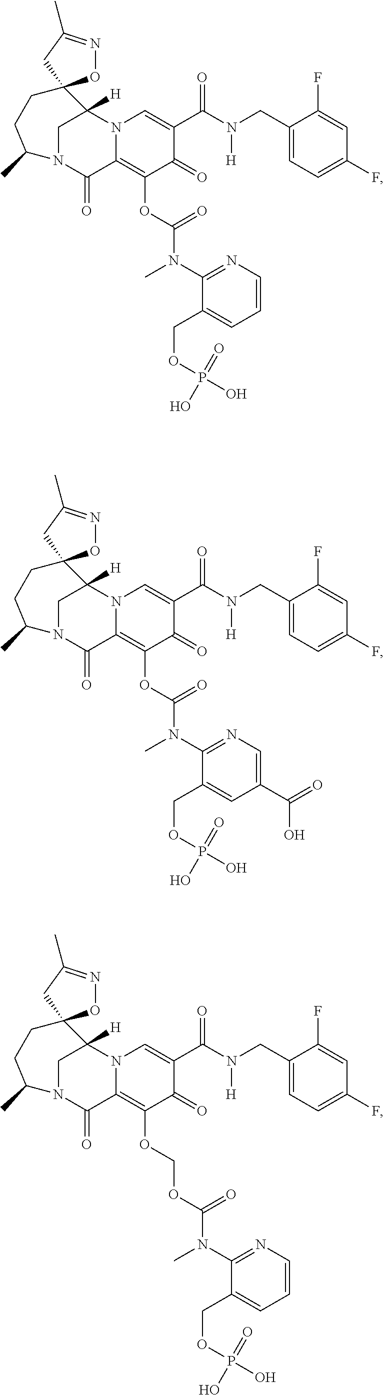 [SMILES: CC1=NO[C@@]2(CC[C@H](C)N3C[C@H]2n2cc(C(=O)NCc4ccc(F)cc4F)c(=O)c(OC(=O)N(C)c4ncccc4COP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(C)c4ncc(C(=O)O)cc4COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)N(C)c4ncccc4COP(=O)(O)O)c2C3=O)C1]

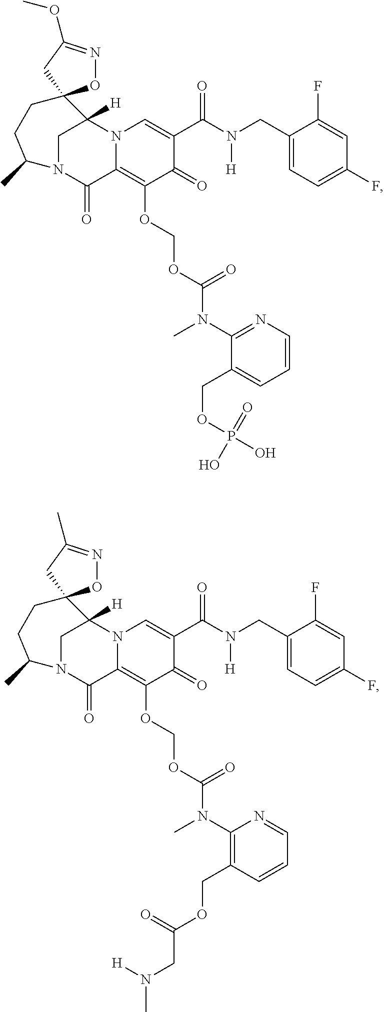 [SMILES: COC1=NO[C@@]2(CC[C@H](C)N3C[C@H]2n2cc(C(=O)NCc4ccc(F)cc4F)c(=O)c(OCOC(=O)N(C)c4ncccc4COP(=O)(O)O)c2C3=O)C1 ; CNCC(=O)OCc1cccnc1N(C)C(=O)OCOc1c2n(cc(C(=O)NCc3ccc(F)cc3F)c1=O)[C@@H]1CN(C2=O)[C@@H](C)CC[C@]12CC(C)=NO2]

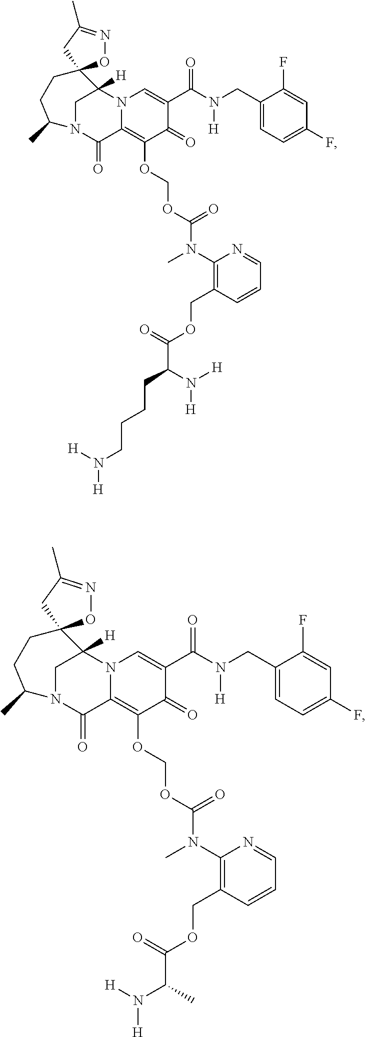 [SMILES: CC1=NO[C@@]2(CC[C@H](C)N3C[C@H]2n2cc(C(=O)NCc4ccc(F)cc4F)c(=O)c(OCOC(=O)N(C)c4ncccc4COC(=O)[C@@H](N)CCCCN)c2C3=O)C1 ; CC1=NO[C@@]2(CC[C@H](C)N3C[C@H]2n2cc(C(=O)NCc4ccc(F)cc4F)c(=O)c(OCOC(=O)N(C)c4ncccc4COC(=O)[C@H](C)N)c2C3=O)C1]

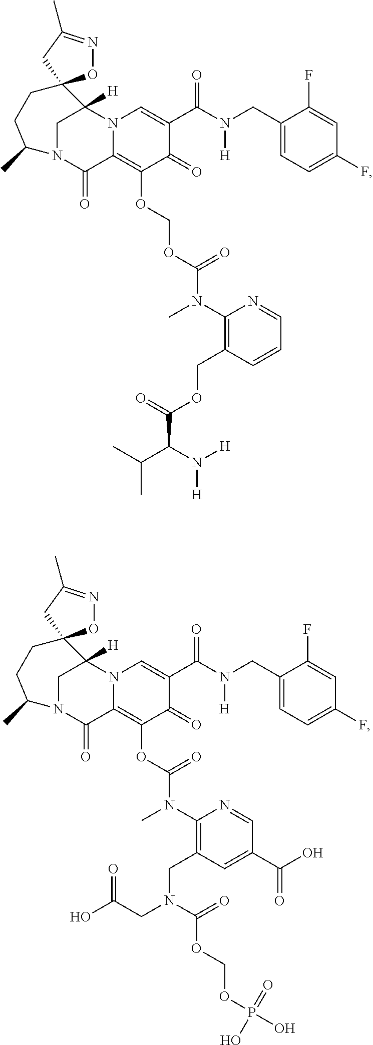 [SMILES: CC1=NO[C@@]2(CC[C@H](C)N3C[C@H]2n2cc(C(=O)NCc4ccc(F)cc4F)c(=O)c(OCOC(=O)N(C)c4ncccc4COC(=O)[C@@H](N)C(C)C)c2C3=O)C1 ; CC1=NO[C@@]2(CC[C@H](C)N3C[C@H]2n2cc(C(=O)NCc4ccc(F)cc4F)c(=O)c(OC(=O)N(C)c4ncc(C(=O)O)cc4CN(CC(=O)O)C(=O)OCOP(=O)(O)O)c2C3=O)C1]

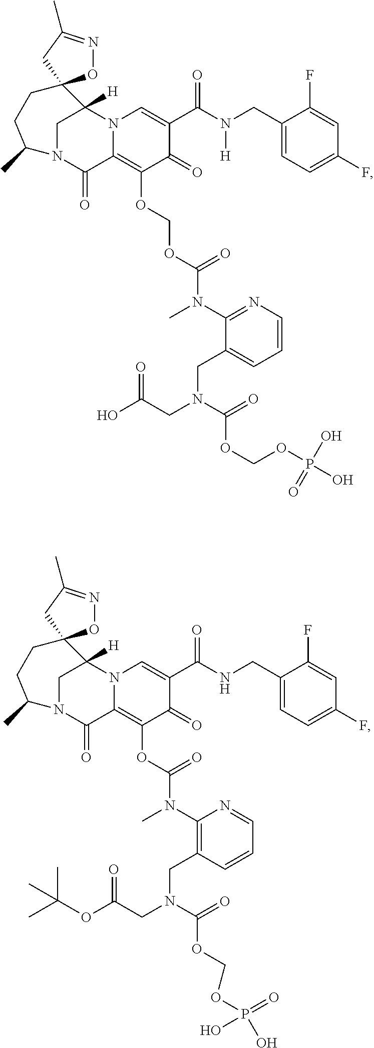 [SMILES: CC1=NO[C@@]2(CC[C@H](C)N3C[C@H]2n2cc(C(=O)NCc4ccc(F)cc4F)c(=O)c(OCOC(=O)N(C)c4ncccc4CN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(C)c4ncccc4CN(CC(=O)OC(C)(C)C)C(=O)OCOP(=O)(O)O)c2C3=O)C1]

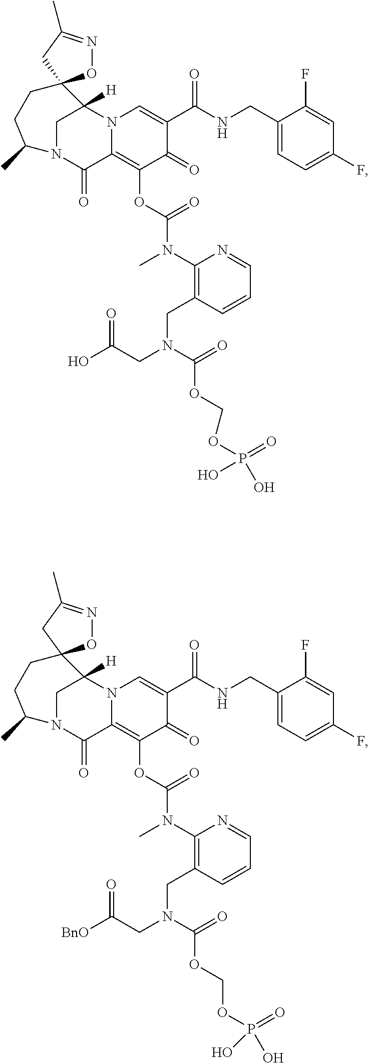 [SMILES: CC1=NO[C@@]2(CC[C@H](C)N3C[C@H]2n2cc(C(=O)NCc4ccc(F)cc4F)c(=O)c(OC(=O)N(C)c4ncccc4CN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(C)c4ncccc4CN(CC(=O)OCc4ccccc4)C(=O)OCOP(=O)(O)O)c2C3=O)C1]

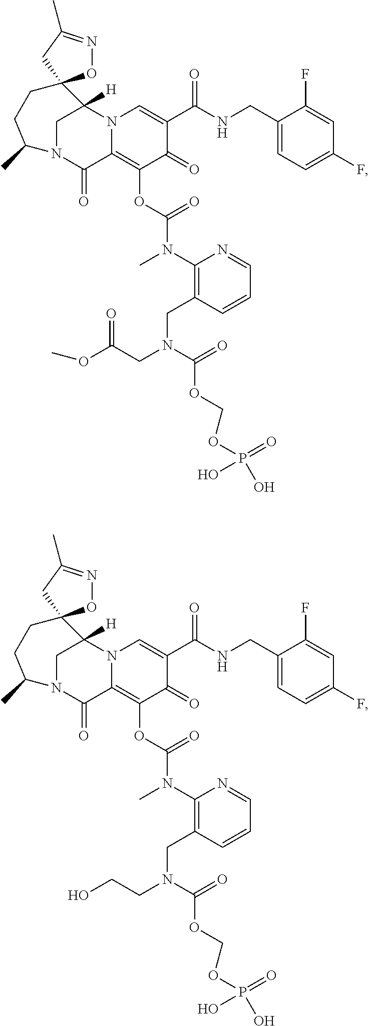 [SMILES: COC(=O)CN(Cc1cccnc1N(C)C(=O)Oc1c2n(cc(C(=O)NCc3ccc(F)cc3F)c1=O)[C@@H]1CN(C2=O)[C@@H](C)CC[C@]12CC(C)=NO2)C(=O)OCOP(=O)(O)O ; CC1=NO[C@@]2(CC[C@H](C)N3C[C@H]2n2cc(C(=O)NCc4ccc(F)cc4F)c(=O)c(OC(=O)N(C)c4ncccc4CN(CCO)C(=O)OCOP(=O)(O)O)c2C3=O)C1]

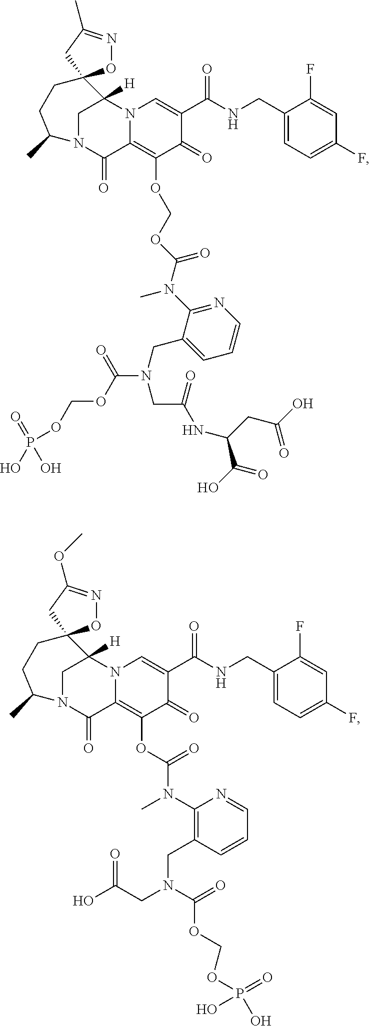 [SMILES: CC1=NO[C@@]2(CC[C@H](C)N3C[C@H]2n2cc(C(=O)NCc4ccc(F)cc4F)c(=O)c(OCOC(=O)N(C)c4ncccc4CN(CC(=O)N[C@@H](CC(=O)O)C(=O)O)C(=O)OCOP(=O)(O)O)c2C3=O)C1 ; COC1=NO[C@@]2(CC[C@H](C)N3C[C@H]2n2cc(C(=O)NCc4ccc(F)cc4F)c(=O)c(OC(=O)N(C)c4ncccc4CN(CC(=O)O)C(=O)OCOP(=O)(O)O)c2C3=O)C1]

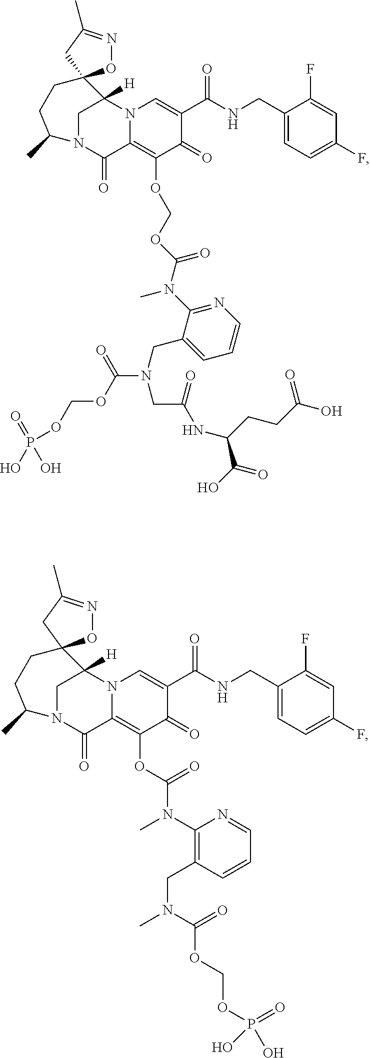 [SMILES: CC1=NO[C@@]2(CC[C@H](C)N3C[C@H]2n2cc(C(=O)NCc4ccc(F)cc4F)c(=O)c(OCOC(=O)N(C)c4ncccc4CN(CC(=O)N[C@@H](CCC(=O)O)C(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(C)c4ncccc4CN(C)C(=O)OCOP(=O)(O)O)c2C3=O)C1]

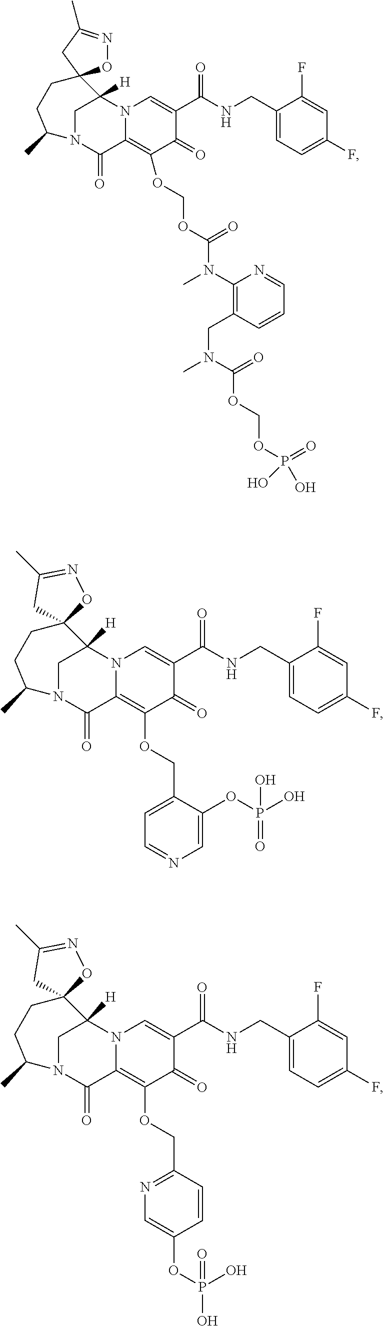 [SMILES: CC1=NO[C@@]2(CC[C@H](C)N3C[C@H]2n2cc(C(=O)NCc4ccc(F)cc4F)c(=O)c(OCOC(=O)N(C)c4ncccc4CN(C)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCc4ccncc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCc4ccc(OP(=O)(O)O)cn4)c2C3=O)C1]

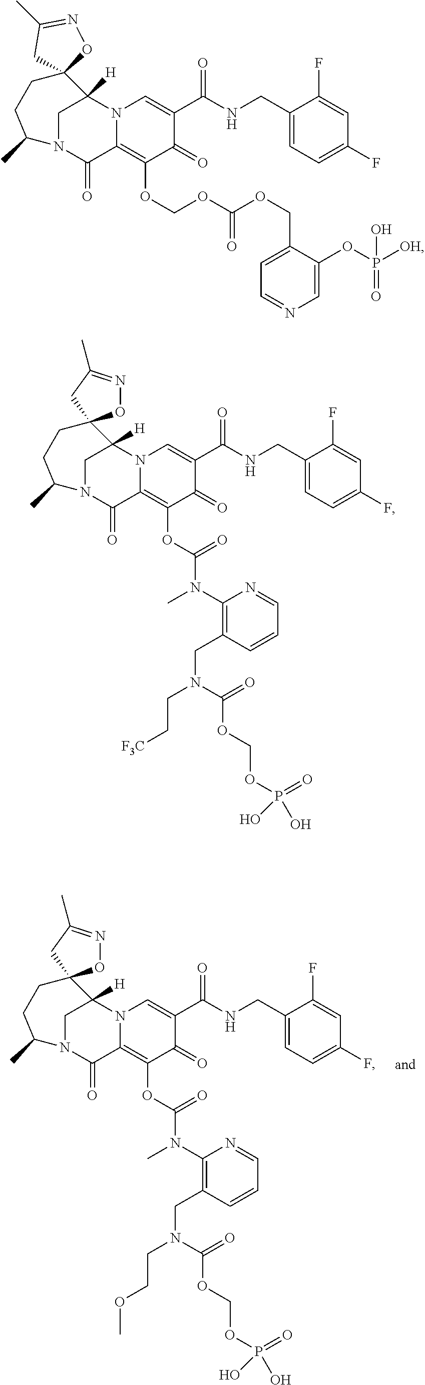 [SMILES: CC1=NO[C@@]2(CC[C@H](C)N3C[C@H]2n2cc(C(=O)NCc4ccc(F)cc4F)c(=O)c(OCOC(=O)OCc4ccncc4OP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(C)c4ncccc4CN(CCC(F)(F)F)C(=O)OCOP(=O)(O)O)c2C3=O)C1 ; COCCN(Cc1cccnc1N(C)C(=O)Oc1c2n(cc(C(=O)NCc3ccc(F)cc3F)c1=O)[C@@H]1CN(C2=O)[C@@H](C)CC[C@]12CC(C)=NO2)C(=O)OCOP(=O)(O)O]

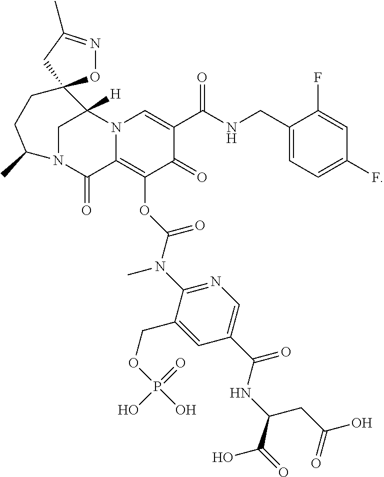 [SMILES: CC1=NO[C@@]2(CC[C@H](C)N3C[C@H]2n2cc(C(=O)NCc4ccc(F)cc4F)c(=O)c(OC(=O)N(C)c4ncc(C(=O)N[C@@H](CC(=O)O)C(=O)O)cc4COP(=O)(O)O)c2C3=O)C1]

In some embodiments of the compound of Formula I, the compound is selected from the group consisting of:

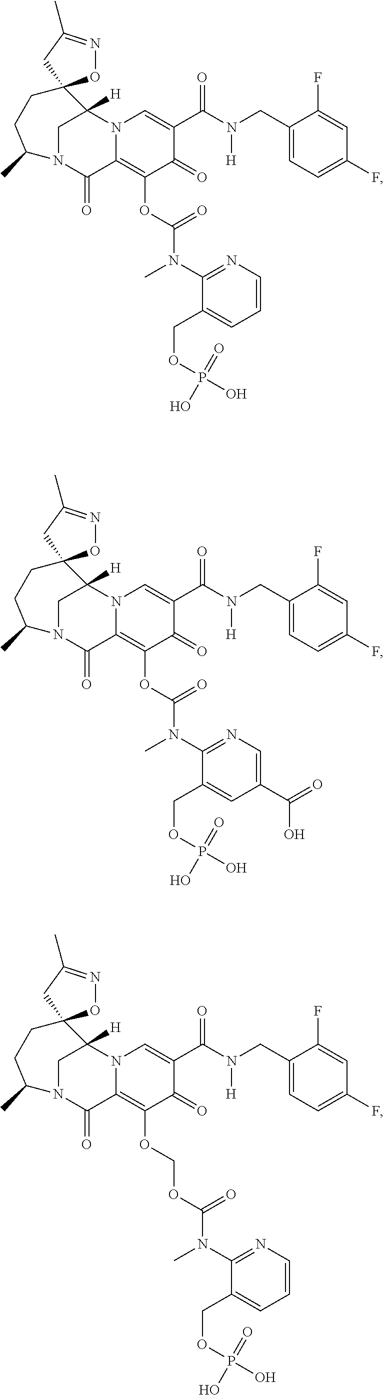 [SMILES: CC1=NO[C@@]2(CC[C@H](C)N3C[C@H]2n2cc(C(=O)NCc4ccc(F)cc4F)c(=O)c(OC(=O)N(C)c4ncccc4COP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(C)c4ncc(C(=O)O)cc4COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)N(C)c4ncccc4COP(=O)(O)O)c2C3=O)C1]

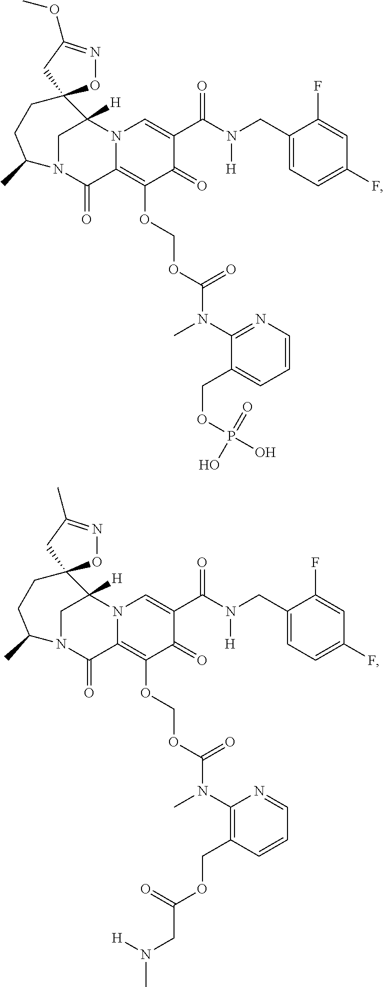 [SMILES: COC1=NO[C@@]2(CC[C@H](C)N3C[C@H]2n2cc(C(=O)NCc4ccc(F)cc4F)c(=O)c(OCOC(=O)N(C)c4ncccc4COP(=O)(O)O)c2C3=O)C1 ; CNCC(=O)OCc1cccnc1N(C)C(=O)OCOc1c2n(cc(C(=O)NCc3ccc(F)cc3F)c1=O)[C@@H]1CN(C2=O)[C@@H](C)CC[C@]12CC(C)=NO2]

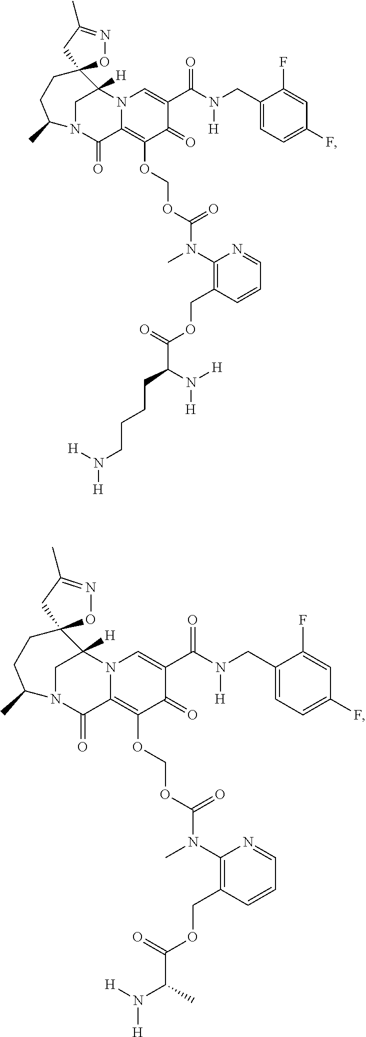 [SMILES: CC1=NO[C@@]2(CC[C@H](C)N3C[C@H]2n2cc(C(=O)NCc4ccc(F)cc4F)c(=O)c(OCOC(=O)N(C)c4ncccc4COC(=O)[C@@H](N)CCCCN)c2C3=O)C1 ; CC1=NO[C@@]2(CC[C@H](C)N3C[C@H]2n2cc(C(=O)NCc4ccc(F)cc4F)c(=O)c(OCOC(=O)N(C)c4ncccc4COC(=O)[C@H](C)N)c2C3=O)C1]

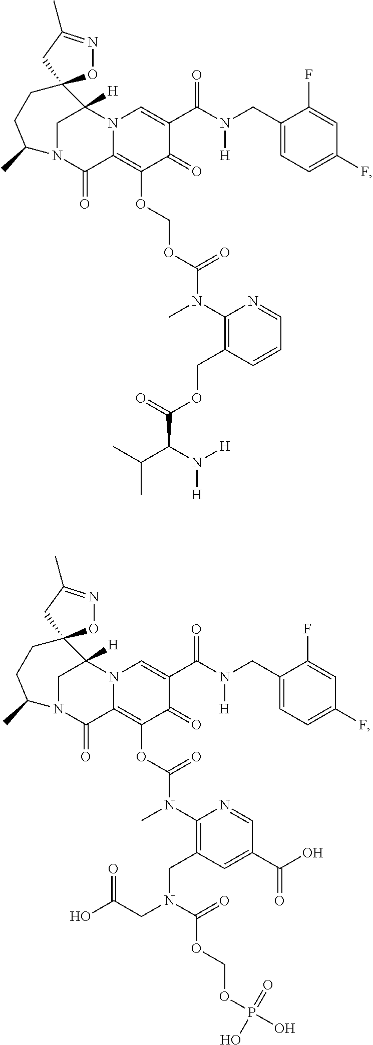 [SMILES: CC1=NO[C@@]2(CC[C@H](C)N3C[C@H]2n2cc(C(=O)NCc4ccc(F)cc4F)c(=O)c(OCOC(=O)N(C)c4ncccc4COC(=O)[C@@H](N)C(C)C)c2C3=O)C1 ; CC1=NO[C@@]2(CC[C@H](C)N3C[C@H]2n2cc(C(=O)NCc4ccc(F)cc4F)c(=O)c(OC(=O)N(C)c4ncc(C(=O)O)cc4CN(CC(=O)O)C(=O)OCOP(=O)(O)O)c2C3=O)C1]

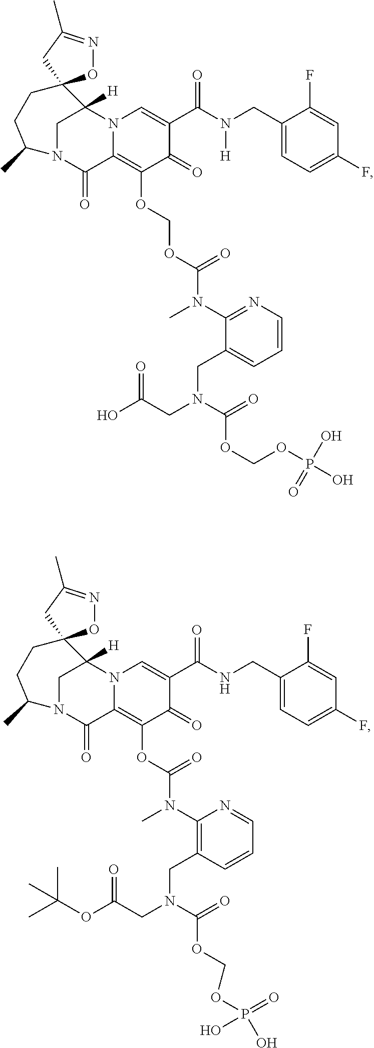 [SMILES: CC1=NO[C@@]2(CC[C@H](C)N3C[C@H]2n2cc(C(=O)NCc4ccc(F)cc4F)c(=O)c(OCOC(=O)N(C)c4ncccc4CN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(C)c4ncccc4CN(CC(=O)OC(C)(C)C)C(=O)OCOP(=O)(O)O)c2C3=O)C1]

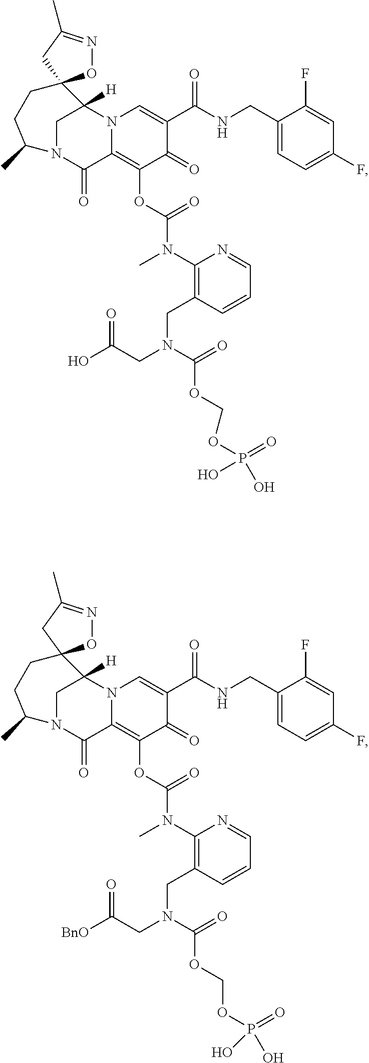 [SMILES: CC1=NO[C@@]2(CC[C@H](C)N3C[C@H]2n2cc(C(=O)NCc4ccc(F)cc4F)c(=O)c(OC(=O)N(C)c4ncccc4CN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(C)c4ncccc4CN(CC(=O)OCc4ccccc4)C(=O)OCOP(=O)(O)O)c2C3=O)C1]

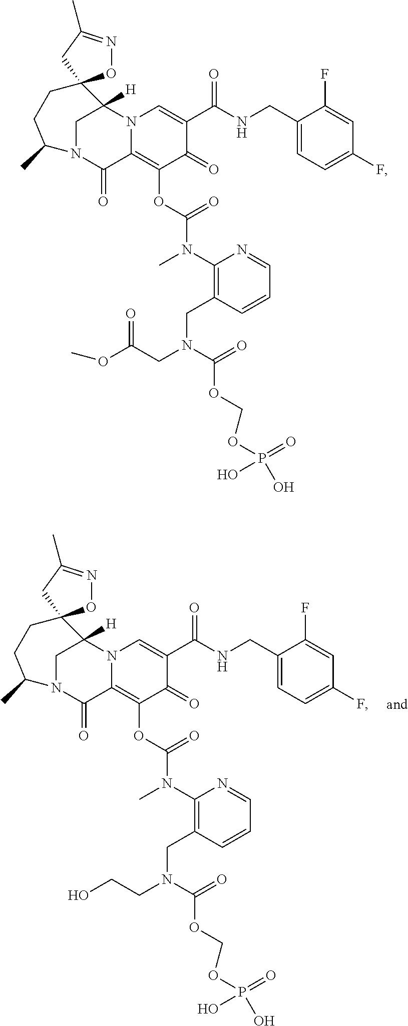 [SMILES: COC(=O)CN(Cc1cccnc1N(C)C(=O)Oc1c2n(cc(C(=O)NCc3ccc(F)cc3F)c1=O)[C@@H]1CN(C2=O)[C@@H](C)CC[C@]12CC(C)=NO2)C(=O)OCOP(=O)(O)O ; CC1=NO[C@@]2(CC[C@H](C)N3C[C@H]2n2cc(C(=O)NCc4ccc(F)cc4F)c(=O)c(OC(=O)N(C)c4ncccc4CN(CCO)C(=O)OCOP(=O)(O)O)c2C3=O)C1]

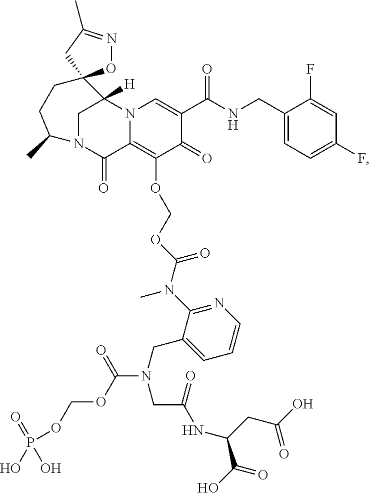 [SMILES: CC1=NO[C@@]2(CC[C@H](C)N3C[C@H]2n2cc(C(=O)NCc4ccc(F)cc4F)c(=O)c(OCOC(=O)N(C)c4ncccc4CN(CC(=O)N[C@@H](CC(=O)O)C(=O)O)C(=O)OCOP(=O)(O)O)c2C3=O)C1]

or a pharmaceutically acceptable salt thereof.

In some embodiments of the compound of Formula I, the compound is selected from the group consisting of:

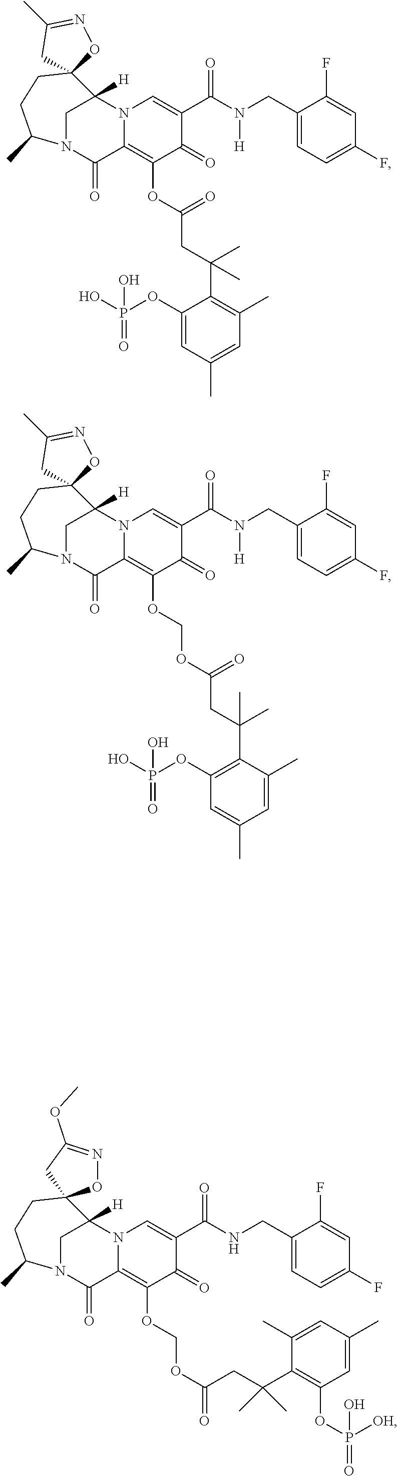 [SMILES: CC1=NO[C@@]2(CC[C@H](C)N3C[C@H]2n2cc(C(=O)NCc4ccc(F)cc4F)c(=O)c(OC(=O)CC(C)(C)c4c(C)cc(C)cc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)c4c(C)cc(C)cc4OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)CC(C)(C)c4c(C)cc(C)cc4OP(=O)(O)O)c2C3=O)C1]

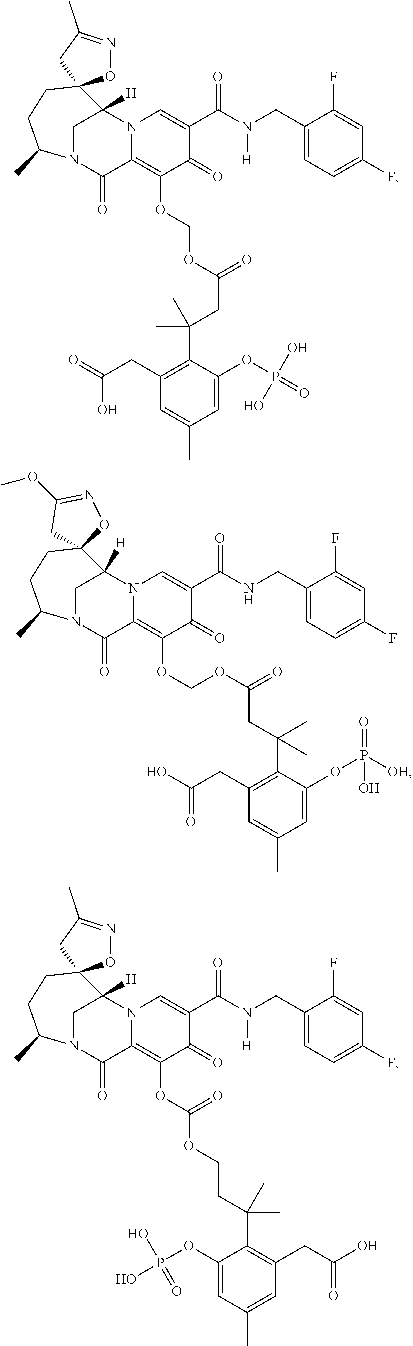 [SMILES: CC1=NO[C@@]2(CC[C@H](C)N3C[C@H]2n2cc(C(=O)NCc4ccc(F)cc4F)c(=O)c(OCOC(=O)CC(C)(C)c4c(CC(=O)O)cc(C)cc4OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)CC(C)(C)c4c(CC(=O)O)cc(C)cc4OP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)OCCC(C)(C)c4c(CC(=O)O)cc(C)cc4OP(=O)(O)O)c2C3=O)C1]

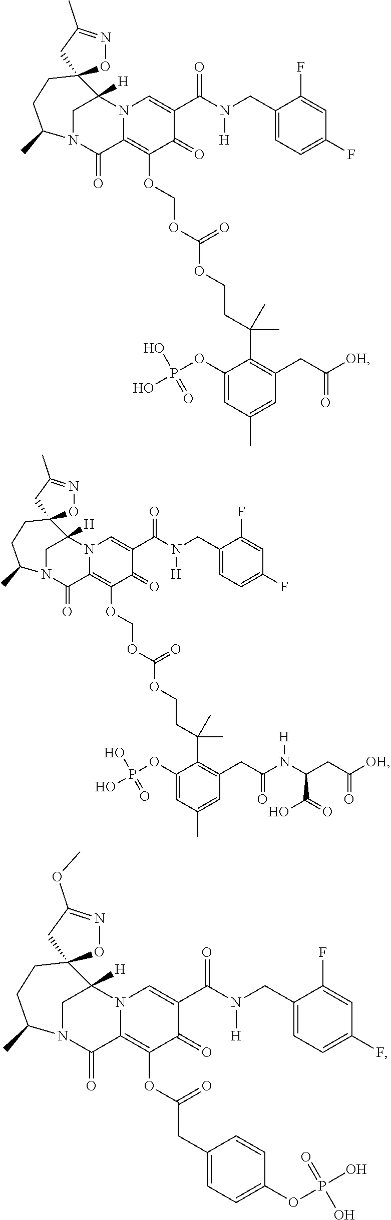 [SMILES: CC1=NO[C@@]2(CC[C@H](C)N3C[C@H]2n2cc(C(=O)NCc4ccc(F)cc4F)c(=O)c(OCOC(=O)OCCC(C)(C)c4c(CC(=O)O)cc(C)cc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCCC(C)(C)c4c(CC(=O)N[C@@H](CC(=O)O)C(=O)O)cc(C)cc4OP(=O)(O)O)c2C3=O)C1 ; COC1=NO[C@@]2(CC[C@H](C)N3C[C@H]2n2cc(C(=O)NCc4ccc(F)cc4F)c(=O)c(OC(=O)Cc4ccc(OP(=O)(O)O)cc4)c2C3=O)C1]

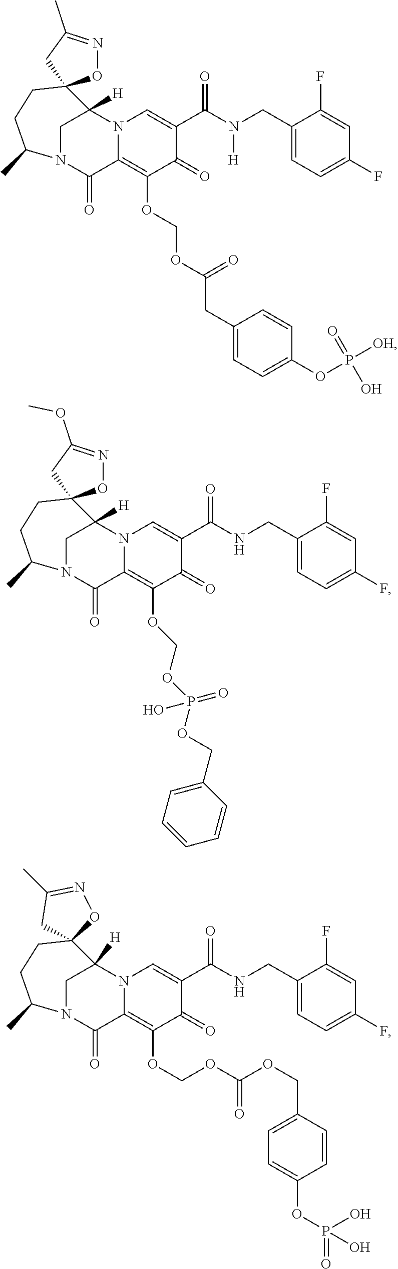 [SMILES: CC1=NO[C@@]2(CC[C@H](C)N3C[C@H]2n2cc(C(=O)NCc4ccc(F)cc4F)c(=O)c(OCOC(=O)Cc4ccc(OP(=O)(O)O)cc4)c2C3=O)C1 ; COC1=NO[C@@]2(CC[C@H](C)N3C[C@H]2n2cc(C(=O)NCc4ccc(F)cc4F)c(=O)c(OCOP(=O)(O)OCc4ccccc4)c2C3=O)C1 ; CC1=NO[C@@]2(CC[C@H](C)N3C[C@H]2n2cc(C(=O)NCc4ccc(F)cc4F)c(=O)c(OCOC(=O)OCc4ccc(OP(=O)(O)O)cc4)c2C3=O)C1]

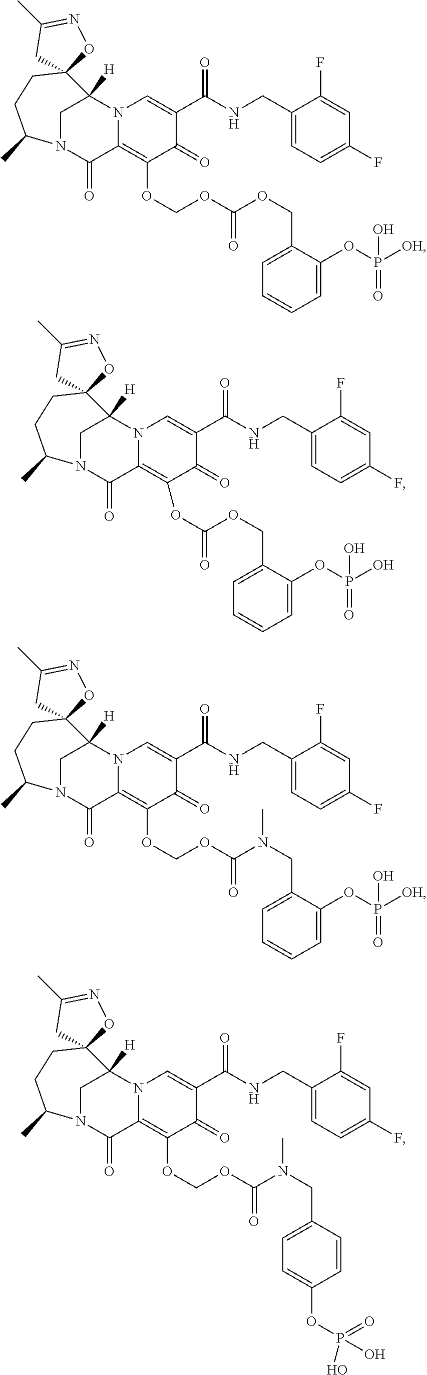 [SMILES: CC1=NO[C@@]2(CC[C@H](C)N3C[C@H]2n2cc(C(=O)NCc4ccc(F)cc4F)c(=O)c(OCOC(=O)OCc4ccccc4OP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)OCc4ccccc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)N(C)Cc4ccccc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)N(C)Cc4ccc(OP(=O)(O)O)cc4)c2C3=O)C1]

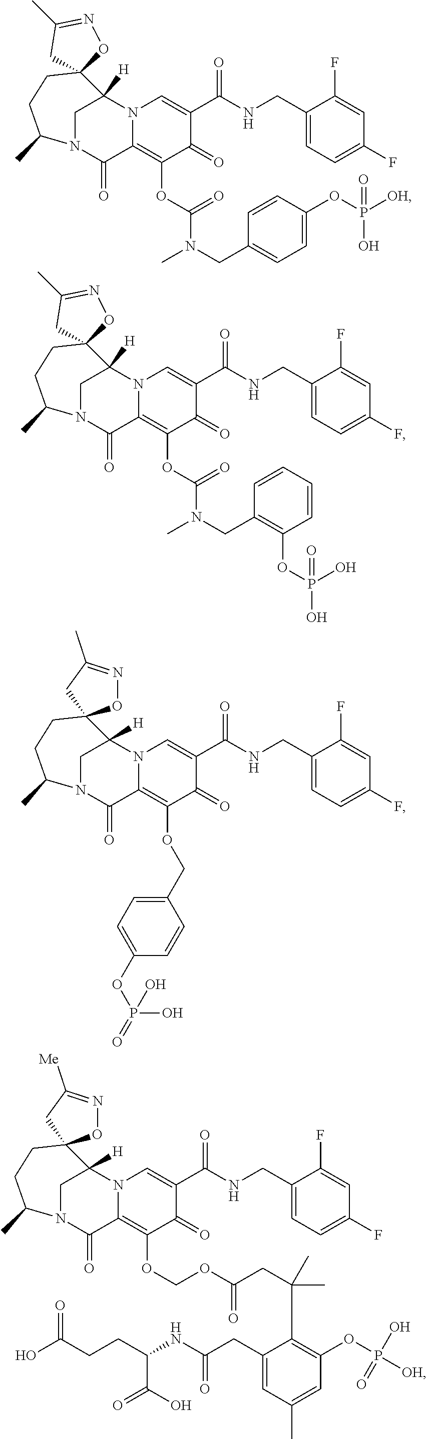 [SMILES: CC1=NO[C@@]2(CC[C@H](C)N3C[C@H]2n2cc(C(=O)NCc4ccc(F)cc4F)c(=O)c(OC(=O)N(C)Cc4ccc(OP(=O)(O)O)cc4)c2C3=O)C1 ; CC1=NO[C@@]2(CC[C@H](C)N3C[C@H]2n2cc(C(=O)NCc4ccc(F)cc4F)c(=O)c(OC(=O)N(C)Cc4ccccc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCc4ccc(OP(=O)(O)O)cc4)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)N[C@@H](CCC(=O)O)C(=O)O)cc(C)cc4OP(=O)(O)O)c2C3=O)C1]

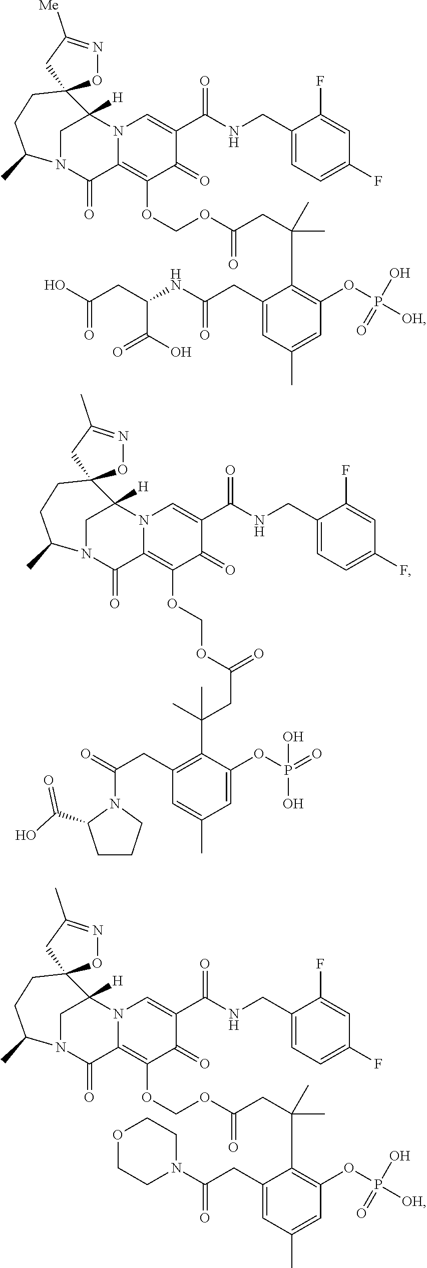 [SMILES: CC1=NO[C@@]2(CC[C@H](C)N3C[C@H]2n2cc(C(=O)NCc4ccc(F)cc4F)c(=O)c(OCOC(=O)CC(C)(C)c4c(CC(=O)N[C@@H](CC(=O)O)C(=O)O)cc(C)cc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)N5CCC[C@@H]5C(=O)O)cc(C)cc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)N5CCOCC5)cc(C)cc4OP(=O)(O)O)c2C3=O)C1]

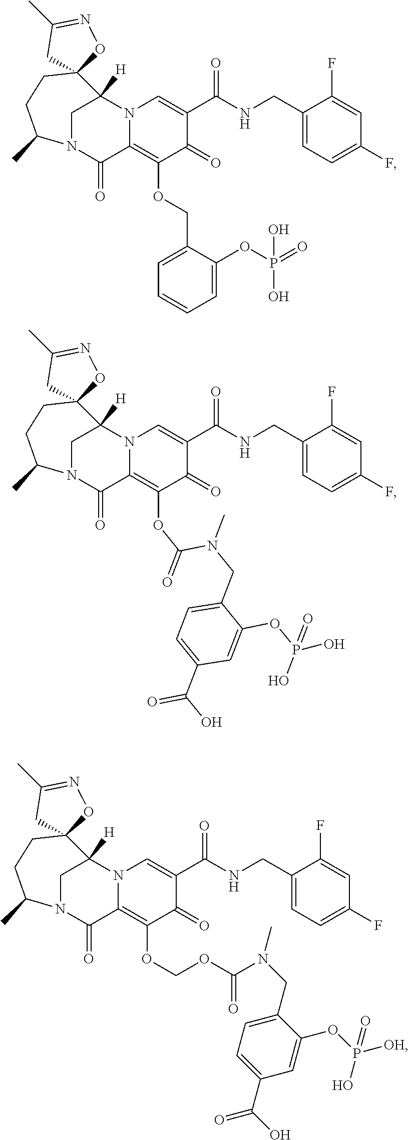 [SMILES: CC1=NO[C@@]2(CC[C@H](C)N3C[C@H]2n2cc(C(=O)NCc4ccc(F)cc4F)c(=O)c(OCc4ccccc4OP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(C)Cc4ccc(C(=O)O)cc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)N(C)Cc4ccc(C(=O)O)cc4OP(=O)(O)O)c2C3=O)C1]

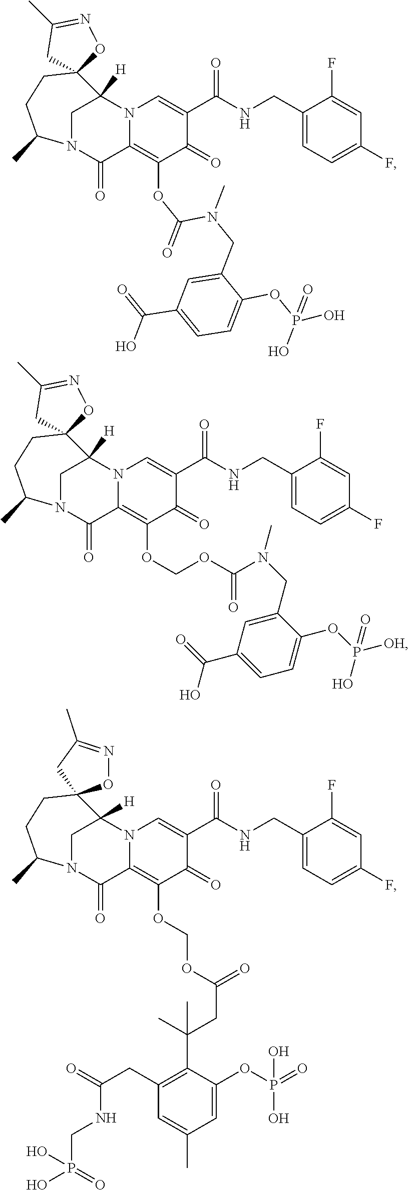 [SMILES: CC1=NO[C@@]2(CC[C@H](C)N3C[C@H]2n2cc(C(=O)NCc4ccc(F)cc4F)c(=O)c(OC(=O)N(C)Cc4cc(C(=O)O)ccc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)N(C)Cc4cc(C(=O)O)ccc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)NCP(=O)(O)O)cc(C)cc4OP(=O)(O)O)c2C3=O)C1]

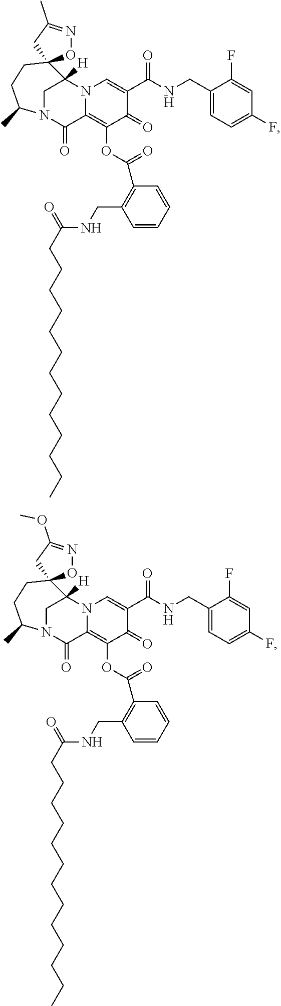

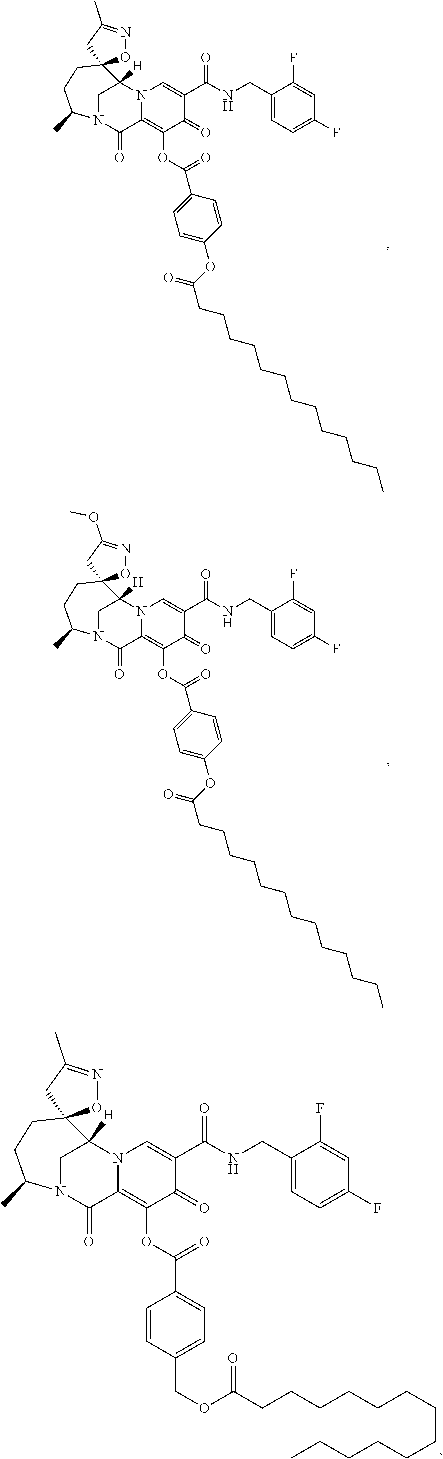

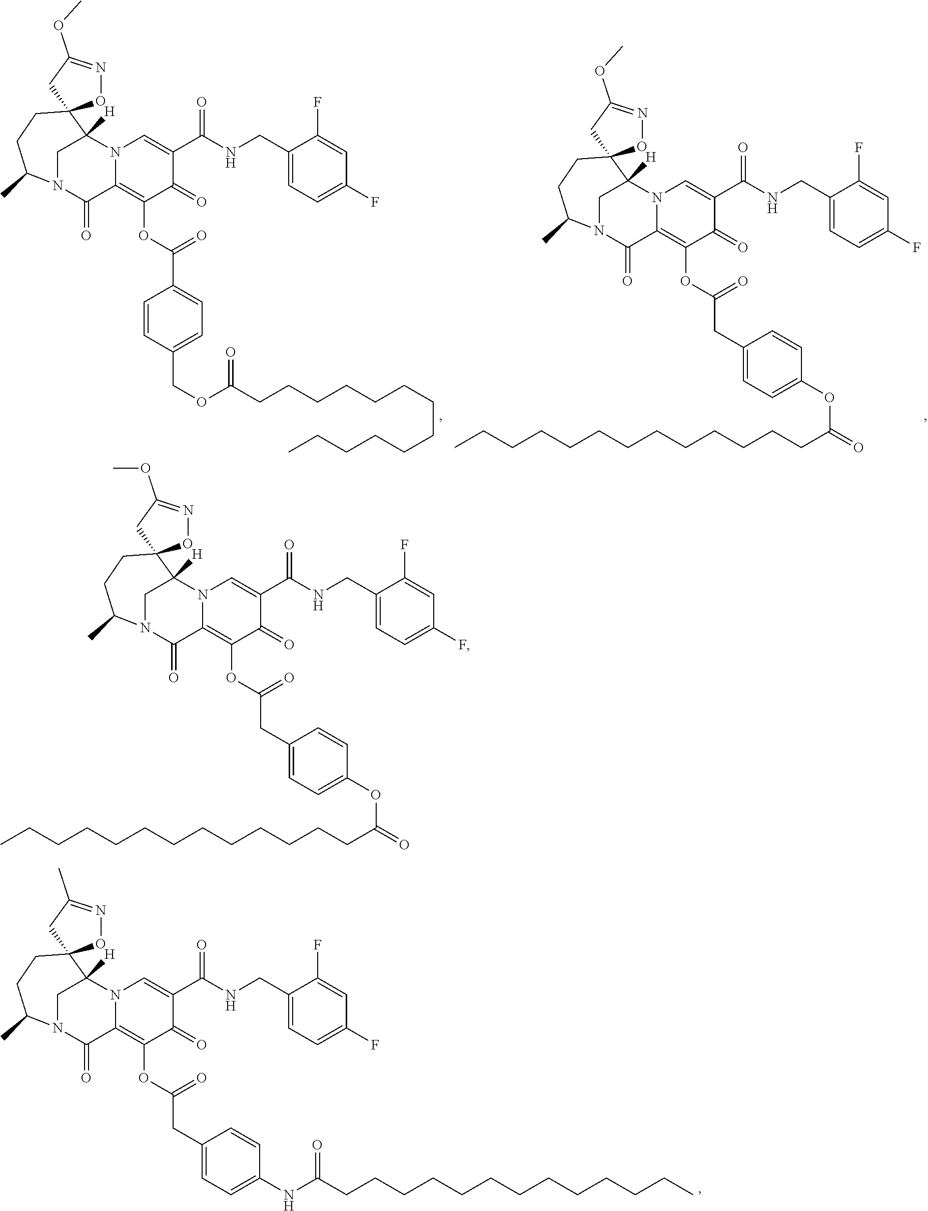

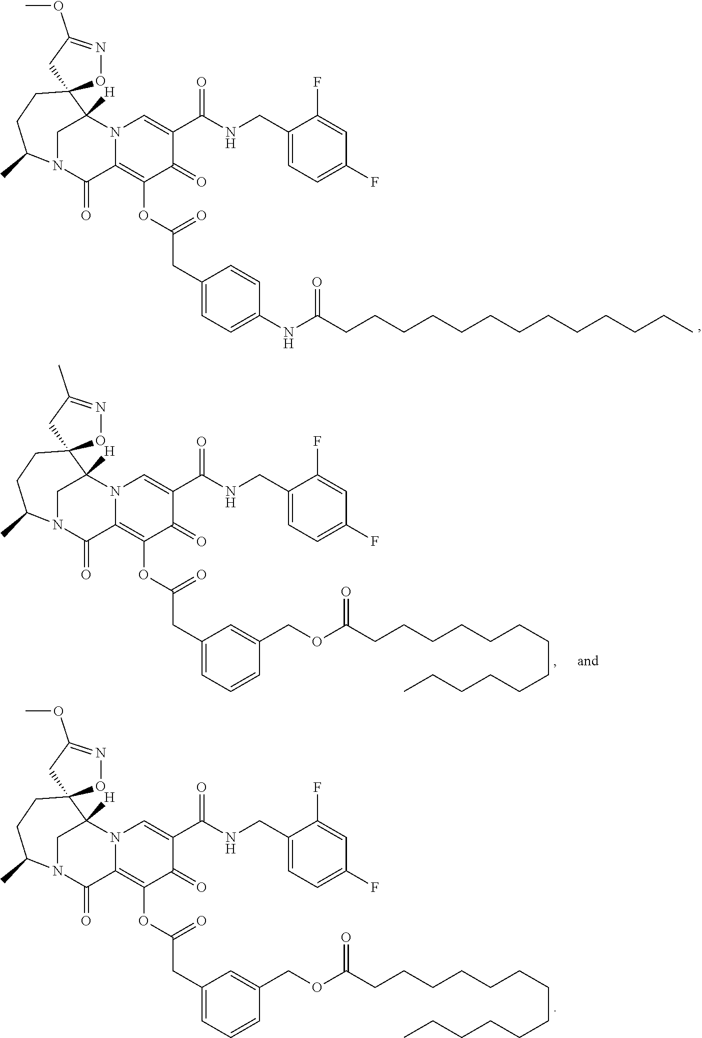

In some embodiments of the compound of Formula I, the compound is selected from the group consisting of:

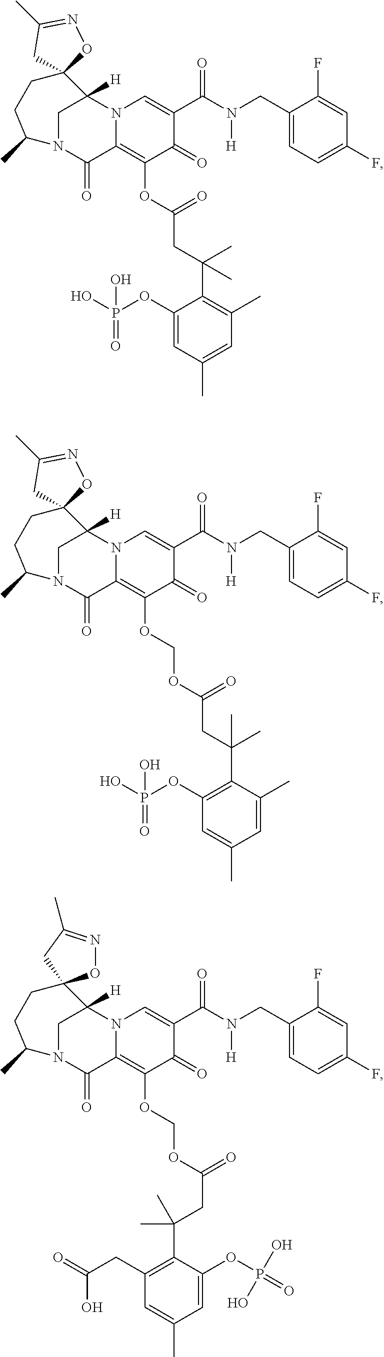 [SMILES: CC1=NO[C@@]2(CC[C@H](C)N3C[C@H]2n2cc(C(=O)NCc4ccc(F)cc4F)c(=O)c(OC(=O)CC(C)(C)c4c(C)cc(C)cc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)c4c(C)cc(C)cc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)O)cc(C)cc4OP(=O)(O)O)c2C3=O)C1]

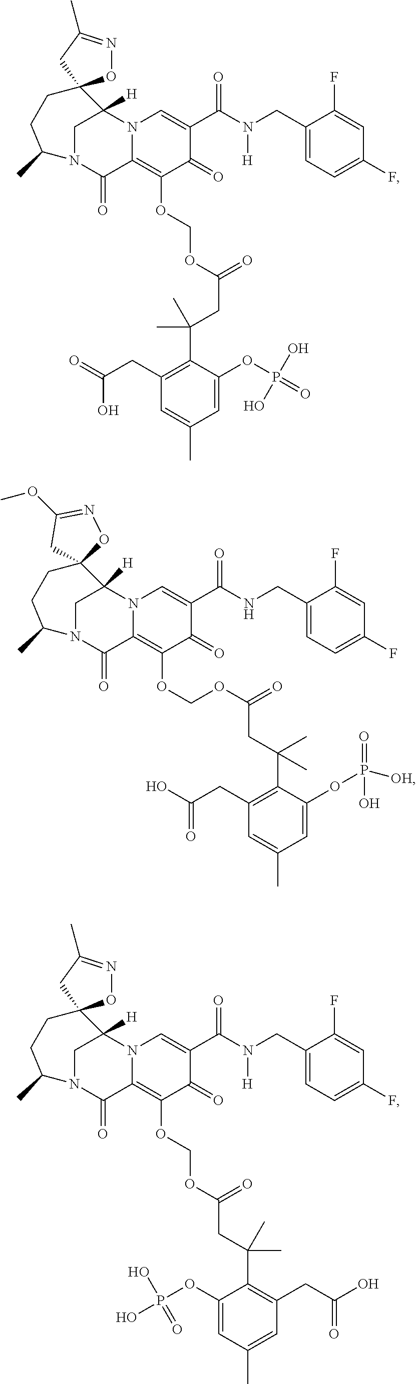 [SMILES: CC1=NO[C@@]2(CC[C@H](C)N3C[C@H]2n2cc(C(=O)NCc4ccc(F)cc4F)c(=O)c(OCOC(=O)CC(C)(C)c4c(CC(=O)O)cc(C)cc4OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)CC(C)(C)c4c(CC(=O)O)cc(C)cc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)O)cc(C)cc4OP(=O)(O)O)c2C3=O)C1]

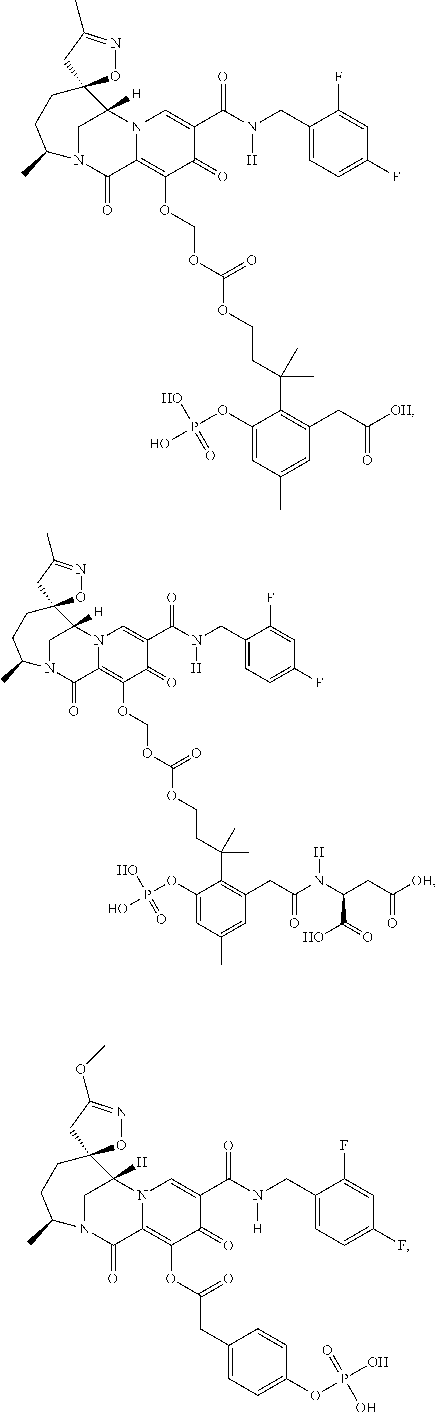 [SMILES: CC1=NO[C@@]2(CC[C@H](C)N3C[C@H]2n2cc(C(=O)NCc4ccc(F)cc4F)c(=O)c(OCOC(=O)OCCC(C)(C)c4c(CC(=O)O)cc(C)cc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCCC(C)(C)c4c(CC(=O)N[C@@H](CC(=O)O)C(=O)O)cc(C)cc4OP(=O)(O)O)c2C3=O)C1 ; COC1=NO[C@@]2(CC[C@H](C)N3C[C@H]2n2cc(C(=O)NCc4ccc(F)cc4F)c(=O)c(OC(=O)Cc4ccc(OP(=O)(O)O)cc4)c2C3=O)C1]

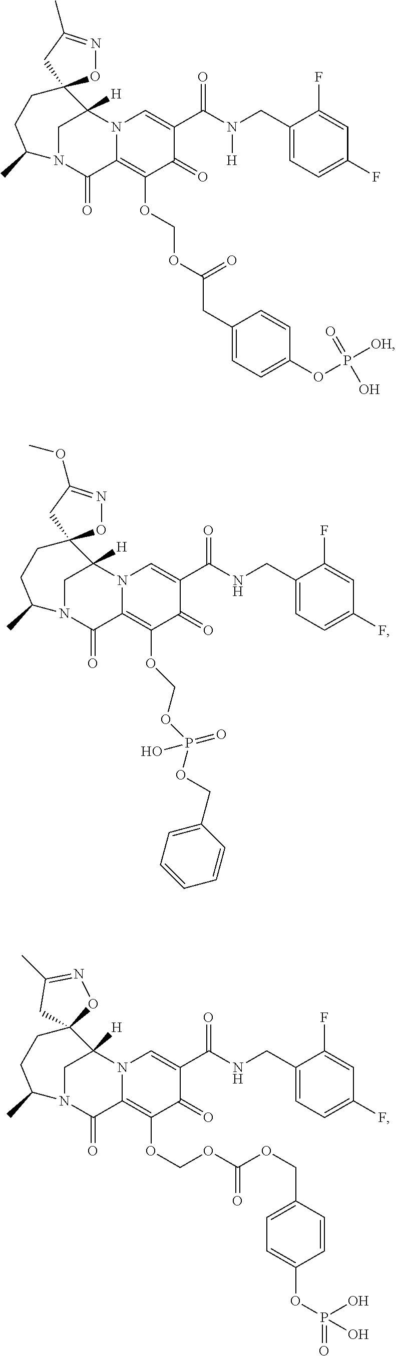 [SMILES: CC1=NO[C@@]2(CC[C@H](C)N3C[C@H]2n2cc(C(=O)NCc4ccc(F)cc4F)c(=O)c(OCOC(=O)Cc4ccc(OP(=O)(O)O)cc4)c2C3=O)C1 ; COC1=NO[C@@]2(CC[C@H](C)N3C[C@H]2n2cc(C(=O)NCc4ccc(F)cc4F)c(=O)c(OCOP(=O)(O)OCc4ccccc4)c2C3=O)C1 ; CC1=NO[C@@]2(CC[C@H](C)N3C[C@H]2n2cc(C(=O)NCc4ccc(F)cc4F)c(=O)c(OCOC(=O)OCc4ccc(OP(=O)(O)O)cc4)c2C3=O)C1]

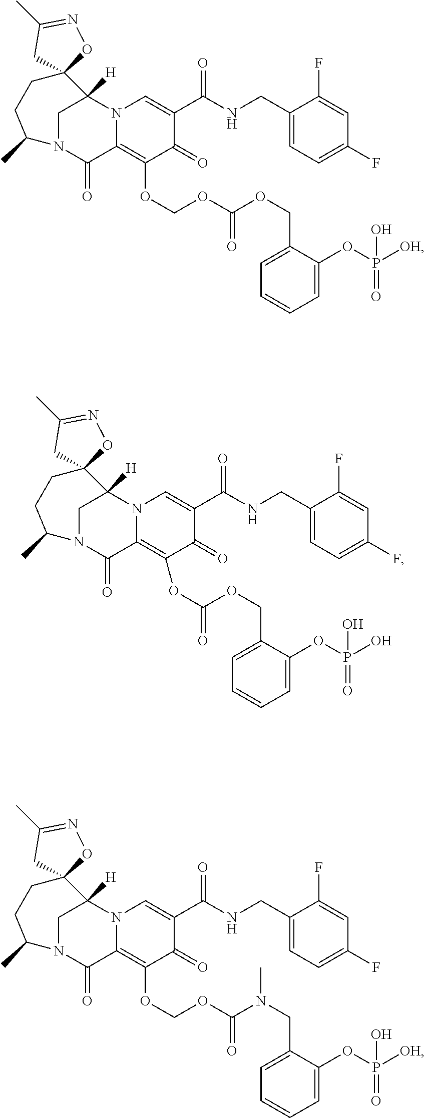 [SMILES: CC1=NO[C@@]2(CC[C@H](C)N3C[C@H]2n2cc(C(=O)NCc4ccc(F)cc4F)c(=O)c(OCOC(=O)OCc4ccccc4OP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)OCc4ccccc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)N(C)Cc4ccccc4OP(=O)(O)O)c2C3=O)C1]

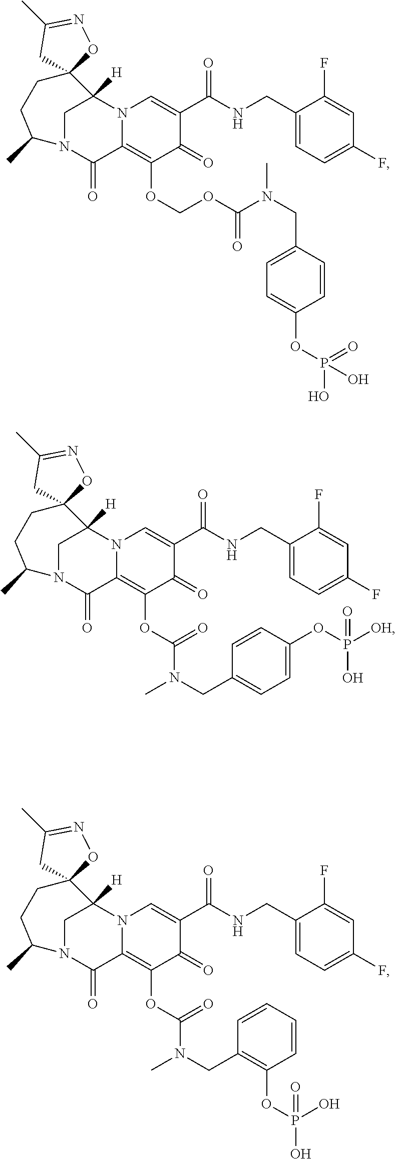 [SMILES: CC1=NO[C@@]2(CC[C@H](C)N3C[C@H]2n2cc(C(=O)NCc4ccc(F)cc4F)c(=O)c(OCOC(=O)N(C)Cc4ccc(OP(=O)(O)O)cc4)c2C3=O)C1 ; CC1=NO[C@@]2(CC[C@H](C)N3C[C@H]2n2cc(C(=O)NCc4ccc(F)cc4F)c(=O)c(OC(=O)N(C)Cc4ccc(OP(=O)(O)O)cc4)c2C3=O)C1 ; CC1=NO[C@@]2(CC[C@H](C)N3C[C@H]2n2cc(C(=O)NCc4ccc(F)cc4F)c(=O)c(OC(=O)N(C)Cc4ccccc4OP(=O)(O)O)c2C3=O)C1]

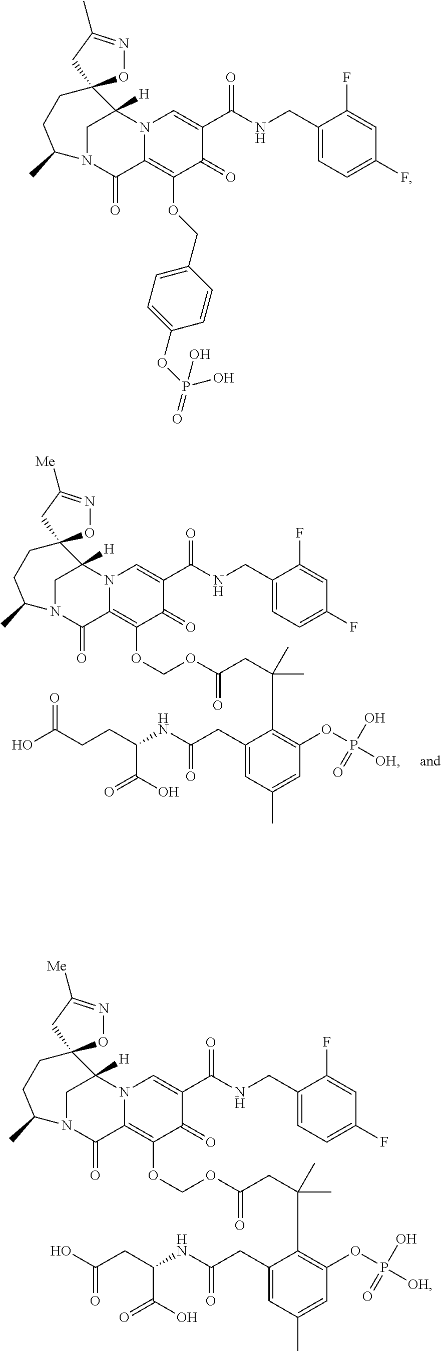 [SMILES: CC1=NO[C@@]2(CC[C@H](C)N3C[C@H]2n2cc(C(=O)NCc4ccc(F)cc4F)c(=O)c(OCc4ccc(OP(=O)(O)O)cc4)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)N[C@@H](CCC(=O)O)C(=O)O)cc(C)cc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)N[C@@H](CC(=O)O)C(=O)O)cc(C)cc4OP(=O)(O)O)c2C3=O)C1]

or a pharmaceutically acceptable salt thereof.

In some embodiments of the compound of Formula I, the compound is selected from the group consisting of:

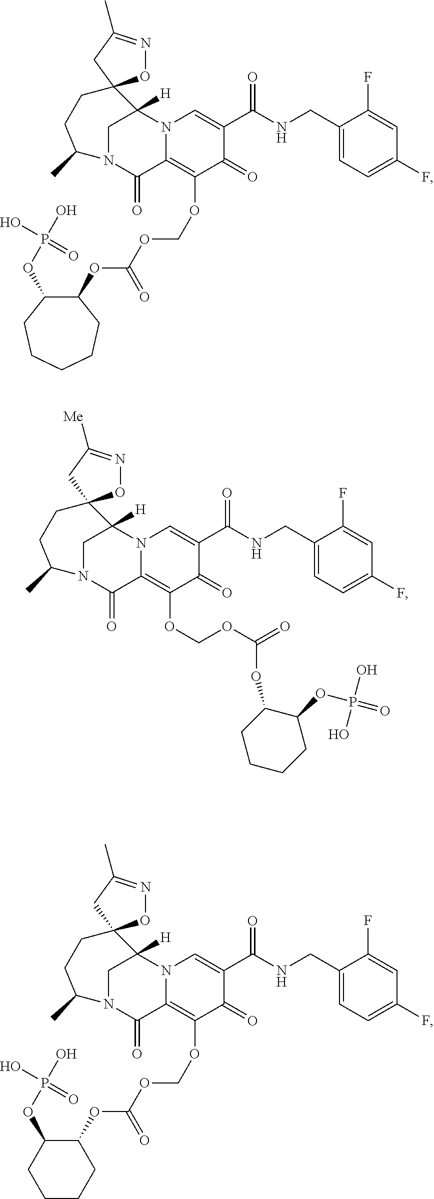 [SMILES: CC1=NO[C@@]2(CC[C@H](C)N3C[C@H]2n2cc(C(=O)NCc4ccc(F)cc4F)c(=O)c(OCOC(=O)O[C@H]4CCCCC[C@@H]4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H]4CCCC[C@@H]4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@@H]4CCCC[C@H]4OP(=O)(O)O)c2C3=O)C1]

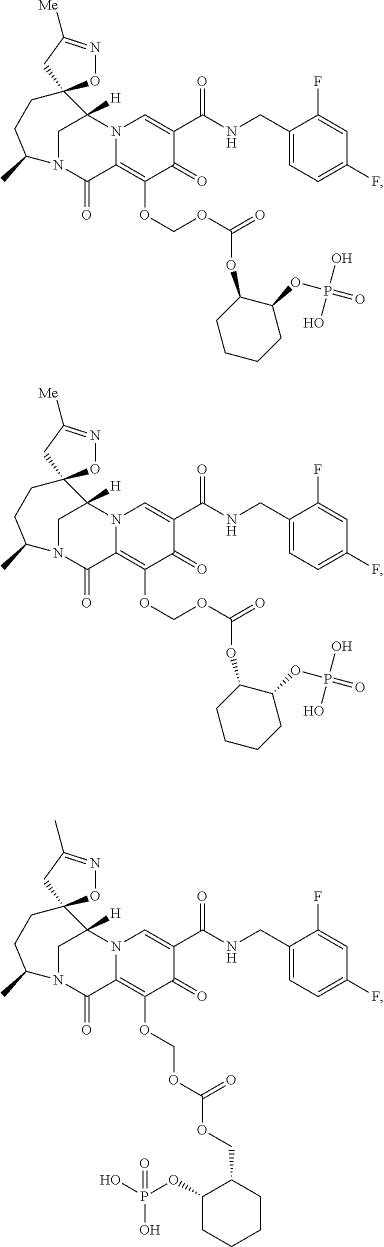 [SMILES: CC1=NO[C@@]2(CC[C@H](C)N3C[C@H]2n2cc(C(=O)NCc4ccc(F)cc4F)c(=O)c(OCOC(=O)O[C@@H]4CCCC[C@@H]4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H]4CCCC[C@H]4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC[C@@H]4CCCC[C@@H]4OP(=O)(O)O)c2C3=O)C1]

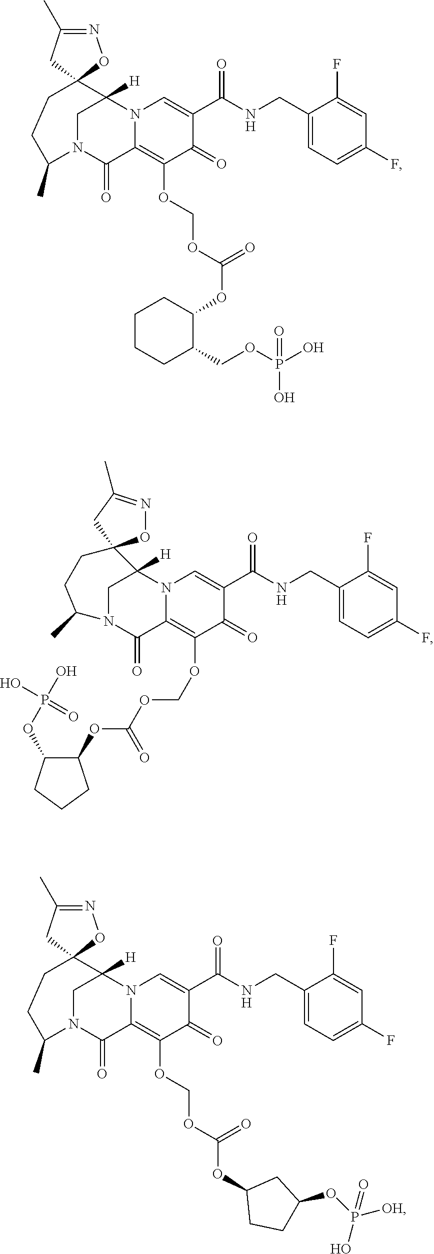 [SMILES: CC1=NO[C@@]2(CC[C@H](C)N3C[C@H]2n2cc(C(=O)NCc4ccc(F)cc4F)c(=O)c(OCOC(=O)O[C@H]4CCCC[C@H]4COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H]4CCC[C@@H]4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@@H]4CC[C@H](OP(=O)(O)O)C4)c2C3=O)C1]

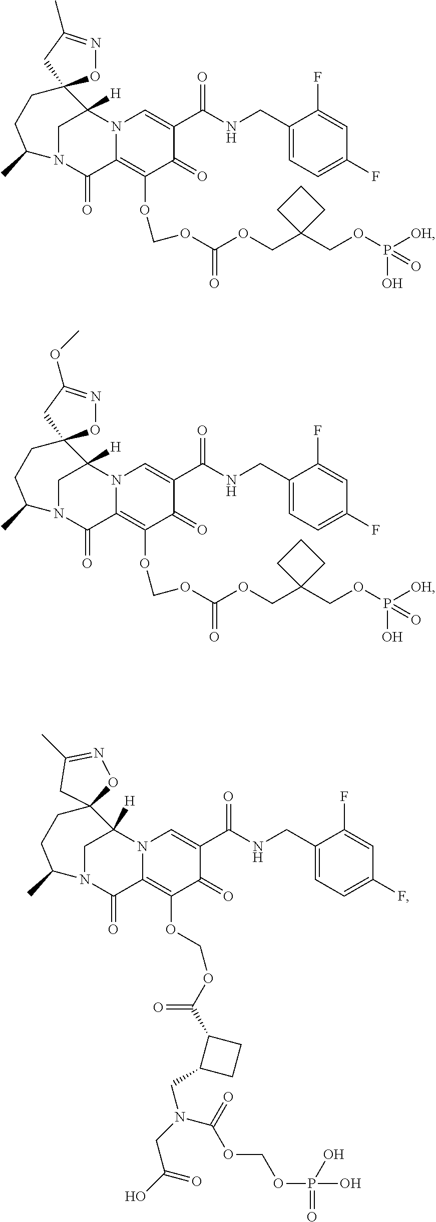 [SMILES: CC1=NO[C@@]2(CC[C@H](C)N3C[C@H]2n2cc(C(=O)NCc4ccc(F)cc4F)c(=O)c(OCOC(=O)OCC4(COP(=O)(O)O)CCC4)c2C3=O)C1 ; COC1=NO[C@@]2(CC[C@H](C)N3C[C@H]2n2cc(C(=O)NCc4ccc(F)cc4F)c(=O)c(OCOC(=O)OCC4(COP(=O)(O)O)CCC4)c2C3=O)C1 ; CC1=NO[C@@]2(CC[C@H](C)N3C[C@H]2n2cc(C(=O)NCc4ccc(F)cc4F)c(=O)c(OCOC(=O)[C@@H]4CC[C@@H]4CN(CC(=O)O)C(=O)OCOP(=O)(O)O)c2C3=O)C1]

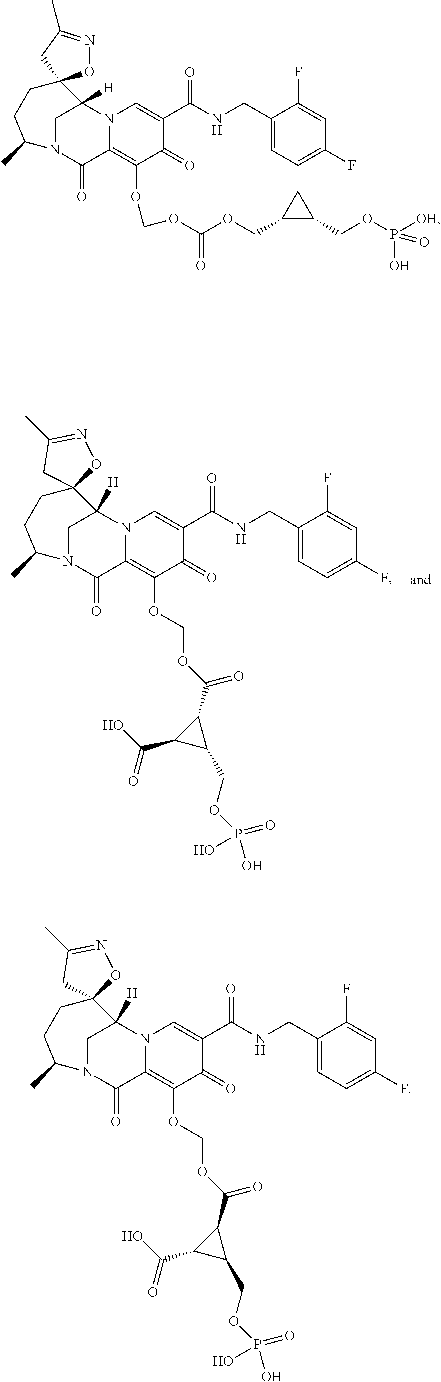 [SMILES: CC1=NO[C@@]2(CC[C@H](C)N3C[C@H]2n2cc(C(=O)NCc4ccc(F)cc4F)c(=O)c(OCOC(=O)OC[C@@H]4C[C@@H]4COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)[C@@H]4[C@H](COP(=O)(O)O)[C@H]4C(=O)O)c2C3=O)C1 ; CC1=NO[C@@]2(CC[C@H](C)N3C[C@H]2n2cc(C(=O)NCc4ccc(F)cc4F)c(=O)c(OCOC(=O)[C@H]4[C@@H](COP(=O)(O)O)[C@@H]4C(=O)O)c2C3=O)C1]

In some embodiments, the disclosure provides a compound of Formula I, or a pharmaceutically acceptable salt thereof, wherein:
R¹ is —(CR^{1A}R^{1B}O)ₐ(Y)_{b}(CR^{1C}R^{1D})_{d}X;
   wherein a is 0 or 1;
   b is 0 or 1;
   d is 0, 1, 2, or 3;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl;
   Y is —C(O)—, —C(O)O—, —C(O)NH—, —C(O)NR^{1L}—, or —P(O)(OH)O—;
      R^{1L} is C₁₋₄alkyl optionally substituted with one or two substituents independently selected from the group consisting of —COOH, —OH, —NH₂ and —CONH₂;
   X is selected from the group consisting of:
      (a) phenyl or pyridyl; wherein the phenyl or pyridyl is optionally substituted with one, two or three substituents independently selected from the group consisting of —OP(O)(OR^{1F})₂, R^{1F}, —COOH, —OCO—C₁₋₂₀alkyl, —NHCO—C₁₋₂₀alkyl,
         wherein each R^{1E} is independently H or phenyl;
         each R^{1F} is independently a C₁₋₃alkyl optionally substituted with one or two substituents independently selected from the group consisting of —COOH, —P(O)(OH)₂, —OP(O)(OR^{1E})₂, —NR^{1G}R^{1H}, —CONR^{1G}R^{1H}—OCOR^{1I}, and —NHCOR^{1I},
         each R^{1G} is independently H, —COOCH₂OP(O)(OH)₂, or C₁₋₄alkyl; wherein the C₁₋₄ alkyl is optionally substituted with one or two substituents independently selected from a group consisting of —COOR^{1M}, —OH, —NH₂, —NR^{1J}R^{1K}, and —CONR^{1J}R^{1K};
         each R^{1H} is independently H, —COOCH₂OP(O)(OH)₂, or C₁₋₄alkyl; wherein the C₁₋₄ alkyl is optionally substituted with one or two substituents independently selected from a group consisting of —COOR^{1M}, —OH, —NH₂, —NR^{1J}R^{1K}, and —CONR^{1J}R^{1K}; or
         optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
         each R^{1I} is independently C₁₋₂₀alkyl optionally substituted with one or two substituents selected independently from —COOH and —NR^{1J}R^{1K};
         each R^{1J} is independently H or C₁₋₃alkyl, wherein the C₁₋₃alkyl is optionally substituted with one or two —COOH;
         each R^{1K} is independently H or C₁₋₃alkyl, wherein the C₁₋₃alkyl is optionally substituted with one or two —COOH; and
         each R^{1M} is independently H or C₁₋₄alkyl, wherein the C₁₋₄alkyl is optionally substituted with phenyl:
      (b) 4 to 7 membered heterocyclyl comprising 1, 2 or 3 heteroatoms selected from N, O, and S; wherein the heterocyclyl is optionally substituted with one, two or three substituents independently selected from the group consisting of C₁₋₄alkyl, —COOC₁₋₄alkyl, oxo, and —COOCH₂OP(O)(OH)₂, wherein the C₁₋₄alkyl is optionally substituted with —OP(O)(OH)₂; and
      (c) C₃₋₆ cycloalkyl optionally substituted with one, two or three substituents independently selected from the group consisting of C₁₋₄alkyl, —COOC₁₋₄alkyl, oxo, and —OP(O)(OH)₂, wherein the C₁₋₄alkyl is optionally substituted with —OP(O)(OH)₂;
R² is C₁₋₃ alkyl or C₁₋₃ alkoxy;
each R³, R⁴, R⁵, R⁶ and R⁷ is independently H or halo; and
R⁸ is H or C₁₋₃alkyl.

In some embodiments, the compounds disclosed herein have therapeutic activity. In some embodiments, the compounds disclosed herein are prodrugs, which upon administration to the human body can be converted to compounds having therapeutic activity. In some embodiments, the compounds disclosed herein are prodrugs of certain compounds disclosed in U.S. application Ser. Nos. 18/296,285, 18/334,588, and 18/334,611, and PCT Application No. PCT/US2023/065401, the disclosures of which are incorporated herein by reference in their entireties. For example, the compounds disclosed herein can be converted to compounds of Formula II:

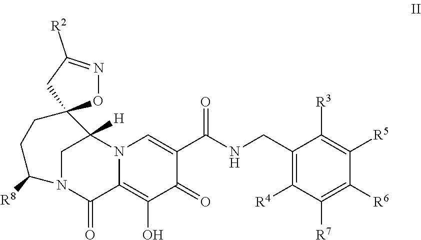

or pharmaceutically acceptable salts thereof, wherein R², R³, R⁴, R⁵, R⁶, R⁷, and R⁸ are as defined according to any other embodiment described herein. Accordingly, the present invention includes a method of converting a compound of Formula I, or a pharmaceutically acceptable salt thereof, to Formula II, or a pharmaceutically acceptable salt thereof, by (1) contacting the compound of Formula I, or a pharmaceutically acceptable salt thereof, with cell-containing aqueous media capable of converting —OR¹ to —OH (e.g., either enzymatically or chemically through acid or base hydrolysis); or (2) administering the compound to a patient whereby the compound of Formula I, or pharmaceutically acceptable salt thereof, is converted to a compound of Formula II, or a pharmaceutically acceptable salt thereof, through biologic pathways (e.g., enzymes) or through contact with biologic fluids and/or tissues conducive to converting —OR¹ to —OH (e.g., through acid or base hydrolysis).

The present invention further provides the treatment or prophylaxis of HIV infection in a patient in need thereof by contacting the patient with a compound of Formula II, or a pharmaceutically acceptable salt thereof, whereby the compound of Formula II, or a pharmaceutically acceptable salt thereof, is generated within the patient upon administration to the patient of a compound of Formula I, or a pharmaceutically acceptable salt thereof. In some embodiments, the compound of Formula II that is generated within the patient is Intermediate B or Intermediate C, as described below in the Example section.

### III. Pharmaceutical Compositions

Compounds provided herein are usually administered in the form of pharmaceutical compositions. Thus, provided herein are also pharmaceutical compositions that comprise one or more of the compounds provided herein or pharmaceutically acceptable salts, isomer, or a mixture thereof and one or more pharmaceutically acceptable vehicles selected from carriers, adjuvants and excipients. The compounds provided herein may be the sole active ingredient or one of the active ingredients of the pharmaceutical compositions. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See. e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In one aspect, provided herein are pharmaceutical compositions comprising a compound provided herein (e.g., a compound of Formula I), or a pharmaceutically acceptable salt thereof, and a pharmaceutically acceptable excipient or carrier. In some embodiments, the pharmaceutical compositions comprise a therapeutically effective amount of a compound provided herein, or a pharmaceutically acceptable salt thereof, and a pharmaceutically acceptable excipient or carrier.

In some embodiments, the pharmaceutical compositions provided herein further comprise one or more (e.g., one, two, three, four, one or two, one to three, or one to four) additional therapeutic agents, or a pharmaceutically acceptable salt thereof. In some embodiments, the pharmaceutical compositions further comprise a therapeutically effective amount of the one or more (e.g., one, two, three, four, one or two, one to three, or one to four) additional therapeutic agents, or a pharmaceutically acceptable salt thereof. In some embodiments, the pharmaceutical compositions further comprise one, tow, three, or four additional therapeutic agents.

The pharmaceutical compositions may be administered in either single or multiple doses. The pharmaceutical compositions may be administered by various methods including, for example, rectal, buccal, intranasal and transdermal routes. In some embodiments, the pharmaceutical compositions may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds provided herein. Administration may be via, for example, capsule or enteric coated tablets. In making the pharmaceutical compositions that include at least one compound provided herein or pharmaceutically acceptable salts, isomer, or a mixture thereof, the active ingredient (such as a compound provided herein) is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose or any combinations thereof. The pharmaceutical compositions can additionally include lubricating agents such as talc, magnesium stearate, and mineral oil; wetting agents; emulsifying and suspending agents; preserving agents such as methyl and propylhydroxy-benzoates; sweetening agents; and flavoring agents; or any combinations thereof.

The pharmaceutical compositions that include at least one compound described herein or pharmaceutically acceptable salts, isomer, or a mixture thereof can be formulated so as to provide quick, sustained or delayed release of the active ingredient (such as a compound provided herein) after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the compounds provided herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or pharmaceutically acceptable salts, isomer, or a mixture thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with materials such as shellac, cetyl alcohol, and cellulose acetate.

Pharmaceutical compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### IV. Methods of Treatment

In one embodiment, methods of treating an HIV (e.g., HIV-1 and/or HIV-2) infection in a human having or at risk of having the infection comprising administering to the human a therapeutically effective amount of a compound of Formula I, or a pharmaceutically acceptable salt thereof, or a pharmaceutical composition of a compound of Formula I, or a pharmaceutically acceptable salt thereof, are provided.

In some embodiments, the methods further comprise administering to the human a therapeutically effective amount of one, two, three, or four additional therapeutic agents. In certain embodiments, the additional therapeutic agent or agents are anti-HIV agents. In particular embodiments, the additional therapeutic agent or agents are HIV protease inhibitors. HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gp120 inhibitors, CCR5 inhibitors, latency reversing agents, capsid polymerization inhibitors, HIV bNAbs (broadly neutralizing HIV antibodies), TLR7 agonists, pharmacokinetic enhancers, other drugs for treating HIV, or combinations thereof.

In some embodiments, the additional therapeutic agent or agents are abacavir, tenofovir alafenamide, tenofovir disoproxil, N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide, or a pharmaceutically acceptable salt thereof. In one embodiment, the additional therapeutic agent or agents are abacavir, tenofovir alafenamide, tenofovir disoproxil, lenacapavir, or a pharmaceutically acceptable salt thereof. In one embodiment, the additional therapeutic agent or agents are abacavir, tenofovir alafenamide, tenofovir disoproxil, lenacapavir, GS-5894, islatravir, or a pharmaceutically acceptable salt thereof. In some embodiments, the additional therapeutic agent or agents are lenacapavir, islatravir. In some embodiments, the additional therapeutic agent is lenacapavir. In some embodiments, the additional therapeutic agent is islatravir.

In another embodiment, a use of a compound of Formula I, or a pharmaceutically acceptable salt thereof, or a pharmaceutical composition of a compound of Formula I, or a pharmaceutically acceptable salt thereof, for treating an HIV (e.g., HIV-1 and/or HIV-2) infection in a human having or at risk of having the infection is provided.

In another embodiment, a compound of Formula I, or a pharmaceutically acceptable salt thereof, or a pharmaceutical composition of a compound of Formula I, or a pharmaceutically acceptable salt thereof, for use in medical therapy is provided.

In another embodiment, a compound of Formula I, or a pharmaceutically acceptable salt thereof, or a pharmaceutical composition of Formula I, or pharmaceutically acceptable salt thereof, for use in treating an HIV infection is provided.

In another embodiment, a compound of Formula I, or a pharmaceutically acceptable salt thereof, or a pharmaceutical composition of a compound of Formula I, or a pharmaceutically acceptable salt thereof for use in a method of treating an HIV infection in a human having or at risk of having the infection, is provided.

In another embodiment, a compound of Formula I, or a pharmaceutically acceptable salt thereof, or a pharmaceutical composition of a compound of Formula I, or a pharmaceutically acceptable salt thereof for use in a method of treating an HIV infection in a human having or at risk of having the infection, is provided wherein said method further comprises administering to the human one, two, three, or four additional therapeutic agents.

In another embodiment, a compound of I, or a pharmaceutically acceptable salt thereof, or a pharmaceutical composition of a compound of Formula I, or a pharmaceutically acceptable salt thereof for use in a method of treating an HIV infection in a human having or at risk of having the infection, is provided wherein said method further comprises administering to the human one, two, three, or four additional therapeutic agents selected from the group consisting of HIV protease inhibitors, HIV non-nucleoside or non-nucleotide inhibitors of reverse transcriptase. HIV nucleoside or nucleotide inhibitors of reverse transcriptase, HIV capsid inhibitors, gp41 inhibitors, CXCR4 inhibitors, gp120 inhibitors, CCR5 inhibitors, latency reversing agents, capsid polymerization inhibitors, HIV bNAbs, TLR7 agonists, pharmacokinetic enhancers, other drugs for treating HIV, or combinations thereof. In one embodiment, the one, two, three, or four additional therapeutic agents are selected from HIV protease inhibitors, HIV non-nucleoside inhibitors of reverse transcriptase, HIV nucleoside or nucleotide inhibitors of reverse transcriptase, latency reversing agents, HIV capsid inhibitors, HIV bNAbs, TLR7 agonists, and combinations thereof.

In another embodiment, a compound of Formula I, or a pharmaceutically acceptable salt thereof, or a pharmaceutical composition of a compound of Formula I, or a pharmaceutically acceptable salt thereof for use in a method of treating an HIV infection in a human having or at risk of having the infection, is provided wherein said method further comprises administering to the human a therapeutically effective amount of tenofovir disoproxil and emtricitabine.

In another embodiment, a compound of Formula I, or a pharmaceutically acceptable salt thereof, or a pharmaceutical composition of a compound of Formula I, or a pharmaceutically acceptable salt thereof for use in a method of treating an HIV infection in a human having or at risk of having the infection, is provided wherein said method further comprises administering to the human a therapeutically effective amount of tenofovir alafenamide and emtricitabine.

In another embodiment, a compound of Formula I, or a pharmaceutically acceptable salt thereof, or a pharmaceutical composition of a compound of I, or a pharmaceutically acceptable salt thereof for use in a method of treating an HIV infection in a human having or at risk of having the infection, is provided wherein said method further comprises administering to the human a therapeutically effective amount of tenofovir disoproxil.

In another embodiment, a compound of Formula I, or a pharmaceutically acceptable salt thereof, or a pharmaceutical composition of a compound of Formula I, or a pharmaceutically acceptable salt thereof for use in a method of treating an HIV infection in a human having or at risk of having the infection, is provided wherein said method further comprises administering to the human a therapeutically effective amount of tenofovir alafenamide.

In another embodiment, a method of using a compound of Formula I, in therapy is provided. In particular, a method of treating the proliferation of the HIV virus, treating AIDS, or delaying the onset of AIDS or ARC symptoms in a mammal (e.g., a human) is provided, comprising administering to the mammal a compound of Formula I, or a pharmaceutically acceptable salt thereof, and a pharmaceutically acceptable excipient.

In another embodiment, a composition comprising a compound of Formula I, or pharmaceutically acceptable salt thereof, and a pharmaceutically acceptable excipient, for use in a method of treating the proliferation of the HIV virus, treating AIDS, or delaying the onset of AIDS or ARC symptoms in a mammal (e.g., a human) is provided.

In one embodiment, a compound of Formula I, or a pharmaceutically acceptable salt thereof, or a pharmaceutical composition of a compound of Formula I, or a pharmaceutically acceptable salt thereof, is provided for use in preventing HIV infection.

For example, in one embodiment, a compound of Formula I, or a pharmaceutically acceptable salt thereof, or a pharmaceutical composition of a compound of Formula I, or a pharmaceutically acceptable salt thereof, is provided for use in pre-exposure prophylaxis (PrEP), i.e., before the exposure of the individual to the HIV virus to prevent HIV infection from taking hold if the individual is exposed to the virus and/or to keep the virus from establishing a permanent infection and/or to prevent the appearance of symptoms of the disease and/or to prevent the virus from reaching detectable levels in the blood.

In another embodiment, the use of a compound of Formula I, or a pharmaceutically acceptable salt thereof, for the manufacture of a medicament for treating an HIV infection in a human being having or at risk of having the infection is disclosed.

In another embodiment, the use of a compound of Formula I, or a pharmaceutically acceptable salt thereof, as a research tool is disclosed.

In another embodiment, an article of manufacture comprising a composition effective to treat an HIV infection, and packaging material comprising a label which indicates that the composition can be used to treat infection by HIV is disclosed. Exemplary compositions comprise a compound of Formula I, or a pharmaceutically acceptable salt thereof.

In still another embodiment, a method of inhibiting the replication of HIV is disclosed. The method comprises exposing the virus to an effective amount of the compound of Formula I, or a pharmaceutically acceptable salt thereof, under conditions where replication of HIV is inhibited.

In another embodiment, the use of a compound of Formula I, or a pharmaceutically acceptable salt thereof, to inhibit the activity of the HIV integrase enzyme is disclosed.

In another embodiment, the use of a compound of Formula I, or a pharmaceutically acceptable salt thereof, to inhibit the replication of HIV is disclosed.

### V. Administration

The compounds of the present disclosure (for example, a compound of Formula I) can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. In some embodiments, the administration is oral, intravenous, subcutaneous, or intramuscular. It will be appreciated that the preferred route may vary with, for example, the condition of the recipient. An advantage of certain compounds disclosed herein is that they are orally bioavailable and can be dosed orally.

A compound of the present disclosure may be administered to an individual in accordance with an effective dosing regimen for a desired period of time or duration, such as at least about one month, at least about 2 months, at least about 3 months, at least about 6 months, or at least about 12 months or longer. In some embodiments, the compound is administered on a daily or intermittent schedule for the duration of the individual's life.

The specific dose level of a compound of the present disclosure for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The dosage may also be described as a total amount of a compound described herein administered per dose. Dosage of a compound of Formula I, or a pharmaceutically acceptable salt or pharmaceutically acceptable tautomer thereof, may be between about 1 mg and 4,000 mg, between about 2,000 to 4,000 mg, between about 1 to 2,000 mg, between about 1 to 1,000 mg, between about 10 to 500 mg, between about 20 to 500 mg, between about 50 to 300 mg, between about 75 to 200 mg, or between about 15 to 150 mg.

The dosage or dosing frequency of a compound of the present disclosure may be adjusted over the course of the treatment, based on the judgment of the administering physician.

The compounds of the present disclosure may be administered to an individual (e.g., a human) in a therapeutically effective amount. In some embodiments, the compound is administered once daily. In some embodiments, the compound is administered once every week. In some embodiments, the compound is administered once every month. In some embodiments, the compound is administered every two months. In some embodiments, the compound is administered every three months. In some embodiments, the compound is administered every four months. In some embodiments, the compound is administered every five months. In some embodiments, the compound is administered every six months. In some embodiments, the compound is administered every seven months. In some embodiments, the compound is administered every eight months. In some embodiments, the compound is administered every nine months. In some embodiments, the compound is administered every ten months. In some embodiments, the compound is administered every eleven months. In some embodiments, the compound is administered every year.

The compounds provided herein can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the compounds provided herein include from about 0.3 mg to about 30 mg per dose, or from about 30 mg to about 300 mg per dose, or from about 0.3 sg to about 30 mg per dose, or from about 30 μg to about 300 μg per dose.

A compound of the present disclosure may be combined with one or more additional therapeutic agents in any dosage amount of the compound of the present disclosure (e.g., from 1 mg to 1000 mg of compound). Therapeutically effective amounts may include from about 0.1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose, or such as from about 0.01 mg per dose to about 1000 mg per dose, or such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose, or such as from about 1 mg per dose to about 1000 mg per dose. Other therapeutically effective amounts of the compound of Formula I are about 1 mg per dose, or about 2, 3, 4, 5, 6, 7, 8, 9, 10, 15, 20, 25, 30, 35, 40, 45, 50, 55, 60, 65, 70, 75, 80, 85, 90, 95, or about 100 mg per dose. Other therapeutically effective amounts of the compound of the present disclosure are about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or about 1000 mg per dose.

In some embodiments, the methods described herein comprise administering to the subject an initial daily dose of about 1 to 500 mg of a compound p herein and increasing the dose by increments until clinical efficacy is achieved. Increments of about 5, 10, 25, 50, or 100 mg can be used to increase the dose. The dosage can be increased daily, every other day, twice per week, once per week, once every two weeks, once every three weeks, or once a month.

When administered orally, the total daily dosage for a human subject may be between about 1 mg and 1,000 mg, between about 10-500 mg/day, between about 50-300 mg/day, between about 75-200 mg/day, or between about 100-150 mg/day. In some embodiments, the total daily dosage for a human subject may be about 100, 200, 300, 400, 500, 600, 700, 800, 900, or 1000 mg/day administered in a single dose. In some embodiments, the total daily dosage for a human subject may be about 200, 30), 400, 500, 600, 70), or 800 mg/day administered in a single dose. In some embodiments, the total daily dosage for a human subject may be about 300, 400, 500, or 600 mg/day administered in a single dose.

In some embodiments, the total daily dosage for a human subject may be about 100 mg/day administered in a single dose. In some embodiments, the total daily dosage for a human subject may be about 150 mg/day administered in a single dose. In some embodiments, the total daily dosage for a human subject may be about 200 mg/day administered in a single dose. In some embodiments, the total daily dosage for a human subject may be about 250 mg/day administered in a single dose. In some embodiments, the total daily dosage for a human subject may be about 300 mg/day administered in a single dose. In some embodiments, the total daily dosage for a human subject may be about 350 mg/day administered in a single dose. In some embodiments, the total daily dosage for a human subject may be about 400 mg/day administered in a single dose. In some embodiments, the total daily dosage for a human subject may be about 450 mg/day administered in a single dose. In some embodiments, the total daily dosage for a human subject may be about 500 mg/day administered in a single dose. In some embodiments, the total daily dosage for a human subject may be about 550 mg/day administered in a single dose. In some embodiments, the total daily dosage for a human subject may be about 600 mg/day administered in a single dose. In some embodiments, the total daily dosage for a human subject may be about 650 mg/day administered in a single dose. In some embodiments, the total daily dosage for a human subject may be about 700 mg/day administered in a single dose. In some embodiments, the total daily dosage for a human subject may be about 750 mg/day administered in a single dose. In some embodiments, the total daily dosage for a human subject may be about 800 mg/day administered in a single dose. In some embodiments, the total daily dosage for a human subject may be about 850 mg/day administered in a single dose. In some embodiments, the total daily dosage for a human subject may be about 900 mg/day administered in a single dose. In some embodiments, the total daily dosage for a human subject may be about 950 mg/day administered in a single dose. In some embodiments, the total daily dosage for a human subject may be about 1000 mg/day administered in a single dose.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a compound disclosed herein is administered once daily in a method disclosed herein. In some embodiments, a compound disclosed herein is administered twice daily in a method disclosed herein.

In some embodiments, a compound disclosed herein is administered once every 10 days. In some embodiments, a compound disclosed herein is administered once every 15 days. In some embodiments, a compound disclosed herein is administered once every 20 days. In some embodiments, a compound disclosed herein is administered once every 10-15 days. In some embodiments, a compound disclosed herein is administered once every 15-20 days. In some embodiments, a compound disclosed herein is administered once every 10-20 days. In some embodiments, a compound disclosed herein is administered once every month. In some embodiments, a compound disclosed herein is administered once every 2 months. In some embodiments, a compound disclosed herein is administered once every 3 months. In some embodiments, a compound disclosed herein is administered once every 4 months. In some embodiments, a compound disclosed herein is administered once every 5 months. In some embodiments, a compound disclosed herein is administered once every 6 months. In some embodiments, a compound disclosed herein is administered once every 8 months. In some embodiments, a compound disclosed herein is administered once every 10 months. In some embodiments, a compound disclosed herein is administered once every year.

The frequency of dosage of the compound of the present disclosure will be determined by the needs of the individual patient and can be, for example, once per day, once per week, once per two weeks, once per month, once per two months, once per three months, once per four months, once per six months, or less. Administration of the compound continues for as long as necessary to treat the HIV infection.

### VI. Kits and Articles of Manufacture

In one aspect, provided herein are kits that comprise a compound provided herein, (e.g., a compound of Formula I), or a pharmaceutically acceptable salt, stereoisomer, prodrug, or solvate thereof, and suitable packaging. In some embodiments, the kit further comprises instructions for use. In some embodiments, the kit comprises a compound provided herein (e.g., a compound of Formula I), or a pharmaceutically acceptable salt, stereoisomer, prodrug, or solvate thereof, and a label and/or instructions for use of the compounds in the treatment of the indications, including the diseases or conditions, described herein.

In some embodiments, the kits further comprise one or more (e.g., one, two, three, four, one or two, one to three, or one to four) additional therapeutic agents, or a pharmaceutically acceptable salt thereof.

In one aspect, provided herein are articles of manufacture that comprise a compound described herein or pharmaceutically acceptable salts, isomer, or a mixture thereof in a suitable container. In some embodiments, the container may be a vial, jar, ampoule, preloaded syringe, or intravenous bag.

### VII. Combination Therapy

In certain embodiments, a method for treating an HIV infection is provided, comprising administering to the human a therapeutically effective amount of a compound disclosed herein, or a pharmaceutically acceptable salt thereof, in combination with a therapeutically effective amount of one, two, three, or four additional therapeutic agents. In one embodiment, a method for treating an HIV infection is provided, comprising administering to the human a therapeutically effective amount of a compound disclosed herein, or a pharmaceutically acceptable salt thereof, in combination with a therapeutically effective amount of one, two, three, or four additional therapeutic agents.

In one embodiment, pharmaceutical compositions comprising a compound disclosed herein, or a pharmaceutically acceptable salt thereof, in combination with one, two, three, or four additional therapeutic agents, and a pharmaceutically acceptable carrier, diluent, or excipient are provided.

In certain embodiments, the present disclosure provides a method for treating an HIV infection, comprising administering to a subject in need thereof a therapeutically effective amount of a compound disclosed herein, or a pharmaceutically acceptable salt thereof, in combination with a therapeutically effective amount of one, two, three, or four additional therapeutic agents which are suitable for treating an HIV infection.

In certain embodiments, a compound disclosed herein, or a pharmaceutically acceptable salt thereof, is combined with one, two, three, four, or more additional therapeutic agents. In certain embodiments, a compound disclosed herein, or a pharmaceutically acceptable salt thereof, is combined with one, two, three, or four additional therapeutic agents. In certain embodiments, a compound disclosed herein, or a pharmaceutically acceptable salt thereof, is combined with two additional therapeutic agents. In other embodiments, a compound disclosed herein, or a pharmaceutically acceptable salt thereof, is combined with three additional therapeutic agents. In further embodiments, a compound disclosed herein, or a pharmaceutically acceptable salt thereof, is combined with four additional therapeutic agents. The one, two, three, four, or more additional therapeutic agents can be different therapeutic agents selected from the same class of therapeutic agents, and/or they can be selected from different classes of therapeutic agents.

### Administration of HIV Combination Therapy

In certain embodiments, a compound disclosed herein is administered with one, two, three, or four additional therapeutic agents. Co-administration of a compound disclosed herein with one, two, three, or four additional therapeutic agents generally refers to simultaneous or sequential administration of a compound disclosed herein and one, two, three, or four additional therapeutic agents, such that therapeutically effective amounts of the compound disclosed herein and the one, two, three, or four additional therapeutic agents are both present in the body of the patient. When administered sequentially, the combination may be administered in two or more administrations.

Co-administration includes administration of unit dosages of the compounds disclosed herein before or after administration of unit dosages of one, two, three, or four additional therapeutic agents. For example, the compound disclosed herein may be administered within seconds, minutes, or hours of the administration of the one, two, three, or four additional therapeutic agents. In some embodiments, a unit dose of a compound disclosed herein is administered first, followed within seconds or minutes by administration of a unit dose of one, two, three, or four additional therapeutic agents. Alternatively, a unit dose of one, two, three, or four additional therapeutic agents is administered first, followed by administration of a unit dose of a compound disclosed herein within seconds or minutes. In other embodiments, a unit dose of a compound disclosed herein is administered first, followed, after a period of hours (e.g., 1-12 hours), by administration of a unit dose of one, two, three, or four additional therapeutic agents. In yet other embodiments, a unit dose of one, two, three, or four additional therapeutic agents is administered first, followed, after a period of hours (e.g., 1-12 hours), by administration of a unit dose of a compound disclosed herein.

In certain embodiments, a kit comprising a compound disclosed herein (e.g., a compound of Formula I, or a pharmaceutically acceptable salt thereof, and one or more (e.g., one, two, three, or four) additional therapeutic agents is provided.

In a specific embodiment, the kit includes a compound disclosed herein, or a pharmaceutically acceptable salt thereof, an HIV nucleoside or nucleotide inhibitor of reverse transcriptase and an HIV capsid inhibitor or an HIV capsid polymerization inhibitor.

### HIV Combination Therapy

In the above embodiments, the additional therapeutic agent or agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors. HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell. CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors. Prolyl endopeptidase inhibitors, Phospholipase A2 inhibitors, pharmacokinetic enhancers, HIV gene therapy, HIV vaccines, anti-HIV peptides, and combinations thereof.

In some embodiments, the additional therapeutic agent or agents are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are chosen from HIV protease inhibitors. HIV non-nucleoside or non-nucleotide inhibitors of reverse transcriptase. HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents are chosen from dolutegravir, cabotegravir, islatravir, darunavir, bictegravir, elsulfavirine, rilpivirine, and lenacapavir, and combinations thereof.

### HIV Combination Drugs

Examples of combination drugs include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenanide), DOVATO® (dolutegravir+lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, lenacapavir+islatravir (oral, injectable), and dual-target HIV-1 reverse transcriptase/nucleocapsid protein 7 inhibitors.

### Other HIV Drugs

Examples of other drugs for treating HIV include, but are not limited to, aspemigrin C, acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ, AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937, GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, MK-8591 (islatravir), NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, xl-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169, BL-008, MK-8122, TMB-607, GRL-02031, and TMC-310911.

Additional examples of HIV protease inhibitors are described, e.g., in U.S. Pat. No. 10,294,234, and U.S. Patent Application Publication Nos. US2020030327 and US2019210978.

### HIV Gag Protein Inhibitors

Examples of HIV Gag protein inhibitors include, but are not limited to, HRF-10071.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, efavirenz, etravirine, lentinan, nevirapine, rilpivirine, ACC-007, ACC-008. AIC-292, F-18, KM-023, PC-1005, M1-TFV, M2-TFV, VM-1500A-LAI, PF-3450074, elsulfavirine (sustained release oral, HIV infection), doravirine+islatravir (fixed dose combination/oral tablet formulation, HIV-1 infection), elsulfavirine (long acting injectable nanosuspension, HIV infection), and elsulfavirine (VM-1500).

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine. OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, islatravir, MK-8583, VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T169, STP-0404, VM-3500, XVIR-110, and ACC-017.

Examples of HIV non-catalytic site, or allosteric, integrase inhibitors (NCINI) include, but are not limited to, CX-05045, CX-05168, and CX-14442.

Additional examples of HIV capsid inhibitors include, but are not limited to, those described in U.S. Patent Application Publication Nos. US20200317689, US20210284642, US2014221356 and US2016016973.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

Examples of gp41 inhibitors include, but are not limited to, albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, C13hmAb, lipuvirtide, PIE-12 trimer and sifuvirtide.

Examples of CD4 attachment inhibitors include, but are not limited to, ibalizumab and CADA analogs.

Examples of gp120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188, N15 peptide, and vMIP (Haimipu).

### HIV Maturation Inhibitors

Examples of HIV maturation inhibitors include, but are not limited to, BMS-955176, GSK-3640254 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators. Smyd2 inhibitors, BET-bromodomain 4 (BRD4) inhibitors (such as ZL-0580, apabetalone), ionomycin, IAP antagonists (inhibitor of apoptosis proteins, such as APG-1387. LBW-242), SMAC mimetics (including TL32711, LCL161, GDC-0917, HGS1029, AT-4)₆, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B, and ubiquitin inhibitors such as largazole analogs, APH-0812, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL4)₀₁), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide. HIV p24 capsid protein inhibitors, lenacapavir (GS-6207), GS-CAI, AVI-621, AVI-101, AVI-201, AVI-301, and AVI-CAN1-15 series, PF-3450074, HIV-1 capsid inhibitors (HIV-1 infection. Shandong University), and compounds described in (GSK WO2019/087016).

Additional examples of capsid inhibitors include, but not limited to, those described in U.S. Patent Application Publication Nos. US2018051005 and US2016108030.

### Cytochrome P450 3 Inhibitors

Examples of Cytochrome P450 3 inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L 1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD 112); CD226 (DNAM-1); Poliovirus receptor (PVR) cell adhesion molecule (PVR, CD155); PVR related immunoglobulin domain containing (PVRIG, CDI 12R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL 16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CDI 12R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD 112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL I); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4). KN-046 (PD-1/CTLA4). MEDI-5752 (CTLA4/PD-1), XmAb-20717 (PD-1/CTLA4), and AK-104 (CTLA4/PD-1).

Examples of inhibitors of PD-L 1 (CD274) or PD-1 (PDCD1) that can be co-administered include without limitation pembrolizumab, nivolumab, cemiplimab, pidilizumab, AMP-224, MED10680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001). BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137. NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF 11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504). TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MEDI6469, MEDI6383, MED10562 (tavolixizumab), MOXR0916, PF-04518600, RG-7888, GSK-3174998, INCAGN1949, BMS-986178, GBR-8383, ABBV-368, and those described in WO2016179517, WO20170%179, WO20170%182, WO20170%281, and WO201808%28.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO201808%28.

### Bi- and Tri-Specific Natural Killer (NK)-Cell Engagers

In various embodiments, the agents as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2EH and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346; Fang et al., Semin Immunol. (2017) 31:37-54. Examples of trispecific NK cell engagers (TRiKE) include, but are not limited to, OXS-3550, HIV-TriKE, and CD16-IL-15-B7H3 TriKe.

Indoleamine-pyrrole-2,3-dioxygenase (IDO1) Inhibitors

In various embodiments, the agents as described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the agents as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-050), GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen). US20140350031 (Janssen). WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, S-540956, litenimod. MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-1CLC, RIBOXXON®, Apoxxim, RIBOXXIM®, IPH-33, MCT-465, MCT-475, and ND-1.1. TLR4 agonists include, but are not limited to, G-100 and GSK-1795091.

### CDK Inhibitors or Antagonists

In some embodiments, the agents described herein are combined with an inhibitor or antagonist of CDK. In some embodiments, the CDK inhibitor or antagonist is selected from the group consisting of VS2-370.

### STING Agonists, RIG-I and NOD2 Modulators

In some embodiments, the agents described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

### LAG-3 and TIM-3 Inhibitors

In certain embodiments, the agents as described herein are combined with an anti-TIM-3 antibody, such as TSR-022, LY-3321367, MBG-453, INCAGN-2390.

In certain embodiments, the antibodies or antigen-binding fragments described herein are combined with an anti LAG-3 (Lymphocyte-activation) antibody, such as relatlimab (ONO-4482), LAG-525, MK-4280, REGN-3767, INCAGN2385.

### Interleukin Agonists

In certain embodiments, the agents described herein are combined with an interleukin agonist, such as IL-2, IL-7, IL-15. IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO-C101, IL-15 Synthorin (pegylated 11-15), P-22339, and a IL-15-PD-1 fusion protein N-809, examples of IL-7 include without limitation CYT-107.

Examples of additional immune-based therapies that can be combined with an agent of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, peginterferon alfa-2b, and RPI-MN.

Phosphatidylinositol 3-kinase (PI3K) INHIBITORS

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530. RV-1729, SAR-245409, SAR-260301, SF-1126, TGR-1202, UCB-5857, VS-5584, XL-765, and ZSTK-474.

### Alpha-4/Beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360. TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, ibalizumab, ibalizumab (second generation), Immuglo, MB-66, clone 3 human monoclonal antibody targeting KLIC (HIV infection), GS-9721, BG-HIV, VRC-HIVMAB091-00-AB.

Various bNAbs may be used. Examples include, but are not limited to, those described in U.S. Pat. Nos. 8,673,307, 9,493,549, 9,783,594, 10,239,935. US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, 1B2530, INC9, 8ANC195, 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al, Nucleic Acids Res., 42 (Database issue): D1 133-9 (2014), Mascola et al., Immunol Rev., 254(1):225-44 (2013), such as 2F5, 4E10, M66.6, CAP206-CH12, 10E81 (all of which bind the MPER of gp41); PG9, PG16, CH01-04 (all of which bind V1V2-glycan), 2G12 (which binds to outer domain glycan); b12, HJ16, CH₁₀₃-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9,783,594; and WO 2012/154312: WO2012/158948: WO 2013/086533; WO 2013/142324; WO2014/063059: WO 2014/089152, WO 2015/048462; WO 2015/103549: WO 2015/117008; WO2016/014484; WO 2016/154003; WO 2016/196975: WO 2016/149710; WO2017/096221; WO 2017/133639: WO 2017/133640, which are hereby incorporated herein by reference in their entireties for all purposes. Additional examples include, but are not limited to, those described in Sajadi et al., Cell. (2018) 173(7):1783-1795; Sajadi et al., J Infect Dis. (2016) 213(1):156-64; Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al., Nucleic Acids Res., 42 (Database issue): D1 133-9 (2014), Mascola et al., Immunol Rev., 254(1):225-44 (2013), such as 2F5, 4E10, M66.6, CAP206-CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1, BiIA-SG, CH01, CH₅₉, C2F5, C4E10, C2F5+C2G12+C4E10, CAP256V2LS, 3BNC117, 3BNC117-LS, 3BNC60, DH270.1, DH270.6, D1D2, 10-1074-LS, C13hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7, VRC-HIVMAB080-00-AB, VRC-HIVMABO60-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include without limitation MGD014, B12BiTe, BiIA-SG, TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J Exp. Med. 2019, 1301).

### Pharmacokinetic Enhancers

Examples of pharmacokinetic enhancers include, but are not limited to, cobicistat and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines. DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e., rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AE-298p, anti-CD40.Env-gp140 vaccine, Ad4-EnvC150, BG505 SOSIP.664 gp140 adjuvanted vaccine, BG505 SOSIP.GT1.1 gp140 adjuvanted vaccine, ChAdOx1.tHIVconsv1 vaccine, CMV-MVA triplex vaccine, ChAdOx1.HTI, Chimigen HIV vaccine, ConM SOSIP.v7 gp140, ALVAC HIV (vCP1521), AIDSVAX BE (gp120), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140[delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PT1, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines. ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C₁₋₆₉₈₀, eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, arenavirus vector-based vaccines (Vaxwave, TheraT), MVA-BN HIV-1 vaccine regimen, mRNA based prophylactic vaccines, VPI-211, multimeric HIV gp120 vaccine (Fred Hutchinson cancer center), TBL-1203HI, CH505 TF chTrimer, CD40.HIVRI.Env vaccine, Drep-HIV-PT-1, mRNA-1644, and mRNA-1574.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a compound disclosed herein, or a pharmaceutically acceptable salt thereof, is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®, lopinavir and ritonavir); COMBIVIR® (zidovudine and lamivudine; AZT+3TC); EPZICOM® (KIVEXA®; abacavir sulfate and lamivudine; ABC+3TC); TRIZIVIR® (abacavir sulfate, zidovudine, and lanivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir, saquinavir mesylate; aldesleukin, zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, an agent disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, an agent disclosed herein, or a pharmaceutical composition thereof, is combined with a first additional therapeutic agent chosen from dolutegravir, cabotegravir, islatravir, darunavir, bictegravir, elsulfavirine, rilpivirine, and lenacapavir and a second additional therapeutic agent chosen from emtricitabine and lamivudine.

In some embodiments, an agent disclosed herein, or a pharmaceutical composition thereof, is combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestreno, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the agents described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-B01, HSPCs overexpressing ALDH1 (LV-800, HIV infection), AGT103-T, and SupT1 cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the agents described herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T Cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy. CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR T-Cell Therapy

In certain embodiments, the agents described herein are combined with a population of TCR-T-cells. TCR-T-cells are engineered to target HIV derived peptides present on the surface of virus-infected cells, for example, ImmTAV.

### B-Cell Therapy

In certain embodiments, the antibodies or antigen-binding fragments described herein are combined with a population of B cells genetically modified to express broadly neutralizing antibodies, such as 3BNC117 (Hartweger et al., J. Exp. Med. 2019, 1301, Moffett et al., Sci. Immunol. 4, eaax0644 (2019) 17 May 2019.

A compound as disclosed herein (e.g., any compound of Formula I) may be combined with one, two, three, or four additional therapeutic agents in any dosage amount of the compound of Formula I (e.g., from 1 mg to 500 mg of compound).

In one embodiment, kits comprising a compound disclosed herein, or a pharmaceutically acceptable salt thereof, in combination with one or more (e.g., one, two, three, one or two, or one to three) additional therapeutic agents are provided.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell. CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MLK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies. PI3K inhibitors, HIV antibodies, and bispecific antibodies, and “antibody-like” therapeutic proteins, and combinations thereof.

In a specific embodiment, the kit includes a compound disclosed herein, or a pharmaceutically acceptable salt thereof, and an HIV nucleoside or nucleotide inhibitor of reverse transcriptase. In a specific embodiment, the kit includes a compound disclosed herein, or a pharmaceutically acceptable salt thereof, and an HIV nucleoside or nucleotide inhibitor of reverse transcriptase and an HIV non-nucleoside inhibitor of reverse transcriptase. In another specific embodiment, the kit includes a compound disclosed herein, or a pharmaceutically acceptable salt thereof, and an HIV nucleoside or nucleotide inhibitor of reverse transcriptase, and an HIV protease inhibiting compound. In an additional embodiment, the kit includes a compound disclosed herein, or a pharmaceutically acceptable salt thereof, an HIV nucleoside or nucleotide inhibitor of reverse transcriptase, an HIV non-nucleoside inhibitor of reverse transcriptase, and a pharmacokinetic enhancer. In certain embodiments, the kit includes a compound disclosed herein, or a pharmaceutically acceptable salt thereof, at least one HIV nucleoside inhibitor of reverse transcriptase, an integrase inhibitor, and a pharmacokinetic enhancer. In another embodiment, the kit includes a compound disclosed herein, or a pharmaceutically acceptable salt thereof, and two HIV nucleoside or nucleotide inhibitors of reverse transcriptase. In a specific embodiment, the kit includes a compound disclosed herein, or a pharmaceutically acceptable salt thereof, an HIV nucleoside or nucleotide inhibitor of reverse transcriptase and an HIV capsid inhibitor. In a specific embodiment, the kit includes a compound disclosed herein, or a pharmaceutically acceptable salt thereof, an HIV nucleoside inhibitor of reverse transcriptase and an HIV capsid inhibitor. In a specific embodiment, the kit includes a compound disclosed herein, or a pharmaceutically acceptable salt thereof, and an HIV capsid inhibitor. In a specific embodiment, the kit includes a compound disclosed herein, or a pharmaceutically acceptable salt thereof, and one, two, three or four HIV bNAbs. In a specific embodiment, the kit includes a compound disclosed herein, or a pharmaceutically acceptable salt thereof, one, two, three or four HIV bNAbs and an HIV capsid inhibitor. In a specific embodiment, the kit includes a compound disclosed herein, or a pharmaceutically acceptable salt thereof, one, two, three or four HIV bNAbs, an HIV capsid inhibitor, and an HIV nucleoside inhibitor of reverse transcriptase.

### HIV Long Acting Therapy

Examples of drugs that are being developed as long acting regimens include, but are not limited to, cabotegravir, rilpivirine, any integrase LA, VM-1500 LAI, maraviroc (LAI), tenofovir implant, islatravir implant, doravirine, raltegravir, and long acting dolutegravir.

### PGP Inhibitors

In some embodiments, the compounds described herein can be used in combination with a P-glycoprotein (PGP) inhibitor. Examples of PGP inhibitors include, but are not limited to, verapamil, dexverapamil, cyclosporine, zosuquidar, laniquidar, elacridar, tariquidar, and encequidar. In some embodiments, the compounds described herein can be used in combination with encequidar. In some embodiments, the compounds described herein can be used in combination with a pharmaceutically acceptable salt of encequidar. In some embodiments, the compounds described herein can be used in combination with a mesylate salt of encequidar.

In some embodiments, the compounds described herein can be co-administered with a P-glycoprotein (PGP) inhibitor. Examples of PGP inhibitors include, but are not limited to, verapamil, dexverapamil, cyclosporine, zosuquidar, laniquidar, elacridar, tariquidar, and encequidar. In some embodiments, the compounds described herein can be co-administered with encequidar. In some embodiments, the compounds described herein can be co-administered with a pharmaceutically acceptable salt of encequidar. In some embodiments, the compounds described herein can be co-administered with a mesylate salt of encequidar.

### VII. Examples

### Intermediate A: (3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide (A)

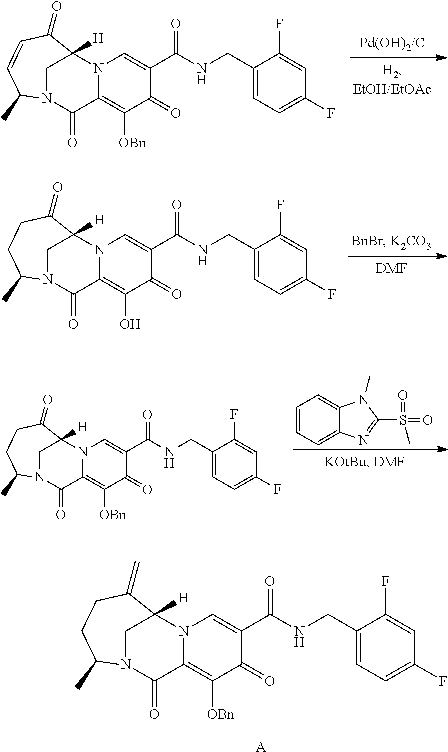

### Step 1: Synthesis of (3S,7R)—N-(2,4-difluorobenzyl)-12-hydroxy-3-methyl-1,6,11-trioxo-1,4,5,6,7,11-hexahydro-3H-2,7-methanopyrido[1,2-a][1,4]diazonine-10-carboxamide

To a solution of (3S,7R)-12-(benzyloxy)-N-(2,4-difluorobenzyl)-3-methyl-1,6,11-trioxo-1,6,7,11-tetrahydro-3H-2,7-methanopyrido[1,2-a][1,4]diazonine-10-carboxamide (17 g, 32.7 mmol) in EtOAc (200 mL) at room temperature was added EtOH (400 mL) followed by 20% Pd(OH)₂/C (50 wt % water, 7.6 g). The resulting mixture was degassed and flushed with nitrogen three times and then degassed and flushed with hydrogen three times before it was hydrogenated under hydrogen balloon for 4 hours. The reaction was then degassed and flushed with nitrogen, diluted with DCM, filtered through Celite®, concentrated and used directly in next step. MS (m/z) 432.124 [M+H]⁺.

### Step 2: Synthesis of (3S,7R)-12-(benzyloxy)-N-(2,4-difluorobenzyl)-3-methyl-1,6,11-trioxo-1,4,5,6,7,11-hexahydro-3H-2,7-methanopyrido[1,2-a][1,4]diazonine-10-carboxamide

(3S,7R)—N-(2,4-difluorobenzyl)-12-hydroxy-3-methyl-1,6,11-trioxo-1,4,5,6,7,11-hexahydro-3H-2,7-methanopyrido[1,2-a][1,4]diazonine-10-carboxamide (33.1 g, 76.7 mmol) from Step 1 was dissolved in DMF (300 mL) at room temperature and K₂CO₃ (16.0 g, 115.0 mmol) and benzyl bromide (13.1 g, 76.7 mmol) were added. The resulting mixture was then heated to 50° C. for 4.5 hours and then cooled to room temperature. The mixture was filtered through a pad of Celite® and the filter cake was rinsed with DMF (100 mL). Combined filtrate was carried directly into the next step. MS (m/z) 554.086 [M+H+MeOH]⁺.

### Step 3: Synthesis of (3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide (A)

The solution of (3S,7R)-12-(benzyloxy)-N-(2,4-difluorobenzyl)-3-methyl-1,6,11-trioxo-1,4,5,6,7,11-hexahydro-3H-2,7-methanopyrido[1,2-a][1,4]diazonine-10-carboxamide (39.7 g, 76.1 mmol) in DMF (350 mL) was immersed into a room temperature water bath. 1-methyl-2-(methylsulfonyl)-1H-benzo[d]imidazole (20.81 g, 99.0 mmol) was added in one portion followed by potassium tert-butoxide (21.36 g, 190 mmol) in 5 portions. The reaction was removed from the water bath and stirred at room temperature for 1.5 hours. The reaction was then quenched slowly with 0.5N HCl in water (180 mL) and extracted with EtOAc three times. The combined organic layer was washed with water and brine, dried over sodium sulfate, filtered and concentrated. The residue was purified by normal phase silica gel chromatography, eluting with 0-100% EtOAc/hexane, to afford intermediate A. MS (m/z) 520.060 [M+H]⁴.

### Intermediate B: Preparation of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (B)

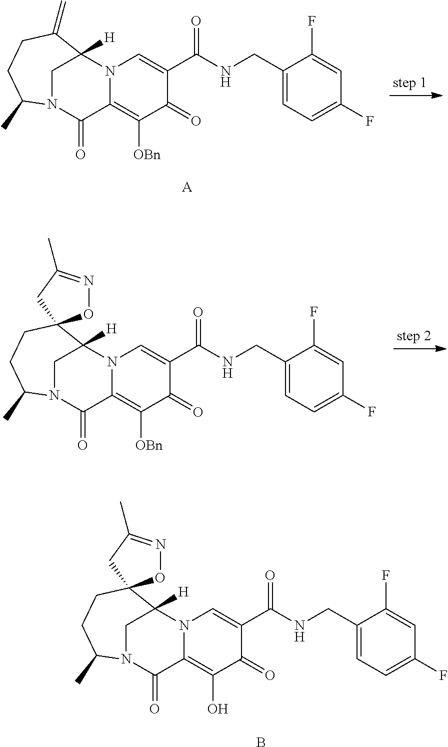

### Step 1: Preparation of (3′S,5S,7′R)-12′-(benzyloxy)-N-(2,4-difluorobenzyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

Into the solution of acetaldehyde oxime (666 mg, 11.3 mmol) in DMF (50 ml) was added N-chlorosuccinimide (1.51 g, 11.3 mmol) at room temperature, then heated to 60° C. for 1 h. After cooling to room temperature, (3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide (Intermediate A) (1.58 g, 3.04 mmol) and triethylamine (1.539 g, 15.2 mmol) were added at room temperature. After stirring at room temperature overnight, the reaction was quenched by adding sat. NaHCO₃ solution. The mixture was extracted with EtOAc, the organic phase was separated and dried over MgSO₄, filtered, concentrated down and purified by silica gel chromatography column (eluting with 0-100% EtOAc/hexane). MS (m/z) 577.135 [M+H]+

### Step 2: Preparation of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (B)

To a solution of (3′S,5S,7′R)-12′-(benzyloxy)-N-(2,4-difluorobenzyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (64.7 mg, 0.112 mmol) in toluene (2 mL) was added TFA (0.5 mL). The reaction mixture was stirred at room temperature overnight. The reaction mixture was concentrated down, and the residue was purified by reverse phase HPLC, eluting with 10-90% acetonitrile in water to give intermediate B. MS (m/z) 487.12 [M+H]⁺. ¹H NMR (400 MHz, Chloroform-d) δ 10.53 (s, 1H), 8.43 (s, 1H), 7.37 (td, J=8.6, 6.3 Hz, 1H), 6.92-6.78 (m, 2H), 4.82-4.70 (m, 1H), 4.67 (t, J=4.8 Hz, 2H), 4.18 (d, J=2.2 Hz, 1H), 3.86 (dd, J=14.9, 1.9 Hz, 1H), 3.72 (dd, J=14.9, 2.7 Hz, 1H), 2.94 (d, J=17.8 Hz, 1H), 2.53 (d, J=17.7 Hz, 1H), 2.06 (s, 3H), 2.04-1.88 (m, 3H), 1.56 (dd, J=14.3, 11.2 Hz, 1H), 1.32 (d, J=6.6 Hz, 3H).

### Intermediate C: Synthesis of (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

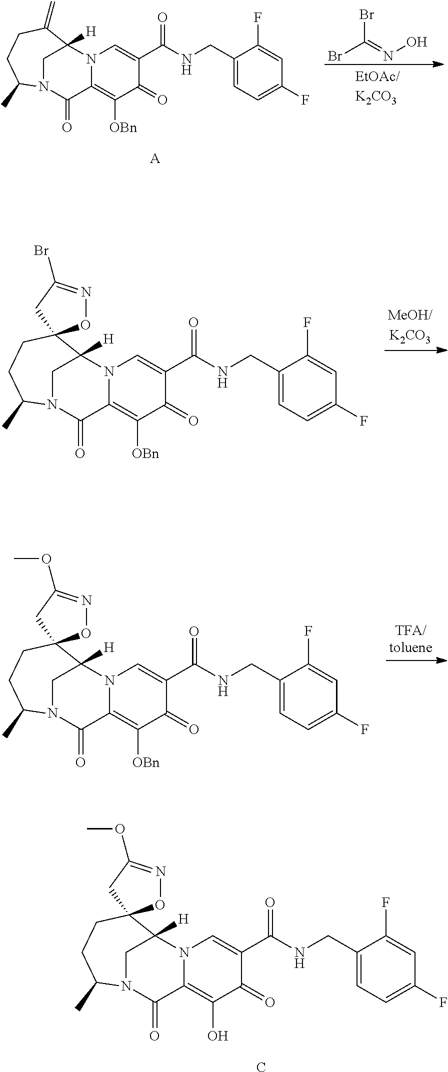

### Step 1: Synthesis of (3′S,5S,7′R)-12′-(benzyloxy)-3-bromo-N-(2,4-difluorobenzyl)-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

To a solution of (3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide (intermediate A) (18.1 g, 34.8 mmol) in EtOAc (350 mL) at 0° C. was added dibromomethanone oxime (21.199 g, 105 mmol) followed by potassium carbonate (28.846 g, 209 mmol). The resulting mixture was stirred overnight before it was diluted with water (300 mL). The layers were separated. The aqueous layer was extracted with EtOAc (200 mL) and the combined organic phase was washed with brine, dried over sodium sulfate, filtered and concentrated. The residue was purified by normal phase silica gel chromatography, eluting with 0-80% EtOAc/Hexane to afford the title compound. MS (m/z) 640.904 [M+H]⁺. ¹H NMR (400 MHz, DMSO) δ 10.41 (t, J=6.0 Hz, 1H), 8.89 (s, 1H), 7.54-7.21 (m, 7H), 7.13-7.05 (m, 1H), 5.30 (d, J=10.5 Hz, 1H), 5.04 (d, J=10.5 Hz, 1H), 4.78 (s, 1H), 4.72-4.54 (m, 3H), 3.75-3.62 (m, 2H), 3.39 (d, J=17.6 Hz, 1H), 3.07 (d, J=17.7 Hz, 1H), 1.92-1.73 (m, 3H), 1.41 (t, J=12.9 Hz, 1H), 1.15 (d, J=6.7 Hz, 3H).

### Step 2: Synthesis of (3′S,5S,7′R)-12′-(benzyloxy-N-(2,4-difluorobenzyl-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

To a mixture of (3′S,5S,7′R)-12′-(benzyloxy)-3-bromo-N-(2,4-difluorobenzyl)-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (23.2 g, 36.2 mmol) in MeOH (450 mL) and DMF (100 mL) at 55° C. was added potassium carbonate (12.4% g, 90.4 mmol). The reaction was stirred for 3 hrs and then cooled to room temperature. To this stirred mixture was added water (1100 mL) and stirring continued for 30 minutes. The mixture was filtered and the filter cake was rinsed with water, collected, and dried to give the title compound. MS (m/z) 593.207 [M+H]⁺. 1H NMR (400 MHz, DMSO) δ 10.41 (t, J=6.0 Hz, 1H), 8.73 (s, 1H), 7.53-7.48 (m, 2H), 7.47-7.30 (m, 4H), 7.25 (ddd, J=10.5, 9.3, 2.6 Hz, 1H), 7.08 (tdd, J=8.5, 2.6, 1.0 Hz, 1H), 5.30 (d, J=10.5 Hz, 1H), 5.03 (d, J=10.5 Hz, 1H), 4.73-4.63 (m, 2H), 4.63-4.49 (m, 2H), 3.82 (s, 3H), 3.65 (d, J=2.0 Hz, 2H), 3.08 (d, J=16.8 Hz, 1H), 2.77 (d, J=16.9 Hz, 1H), 1.96-1.71 (m, 3H), 1.38 (dd, J=15.5, 10.2 Hz, 1H), 1.15 (d, J=6.6 Hz, 3H).

### Step 3: Synthesis of (3′,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (C)

To a solution of (3′S,5S,7′R)-12′-(benzyloxy)-N-(2,4-difluorobenzyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (19 g, 32.1 mmol) in toluene (64 mL) at room temperature was added TFA (32 mL) and stirred for 16 hrs. The solvents were removed by rotatory evaporator. The resulting residue was treated with EtOAc (60 mL) and concentrated, repeating three times. The residue was stirred with a mixture of toluene (42 mL)/MeOH (42 mL)/EtOAc (42 mL) at 70° C. for 1 hr and room temperature for 2 hours. The mixture was then filtered and the filter cake was rinsed with EtOAc, collected and dried to give title compound. MS (m/z) 503.263 [M+H]⁺. 1H NMR (400 MHz, DMSO) δ 10.99 (s, 1H), 10.34 (t, 3=5.9 Hz, 1H), 8.64 (s, 1H), 7.42 (td, J=8.7, 6.6 Hz, 1H), 7.25 (ddd, J=10.5, 9.3, 2.6 Hz, 1H), 7.08 (tdd, J=8.6, 2.6, 1.0 Hz, 1H), 4.74 (s, 1H), 4.63-4.47 (m, 3H), 3.81 (s, 3H), 3.80-3.67 (m, 2H), 2.93 (d, J=16.9 Hz, 1H), 2.70 (d, J=16.9 Hz, 1H), 1.95-1.76 (m, 3H), 1.41-1.31 (m, 1H), 1.19 (d, J=6.8 Hz, 3H).

### Intermediate D: tert-butyl N-((((di-tert-butoxyphosphoryl)oxy)methoxy)carbonyl)-N-((2-(methylamino)pyridin-3-yl)methyl)glycinate

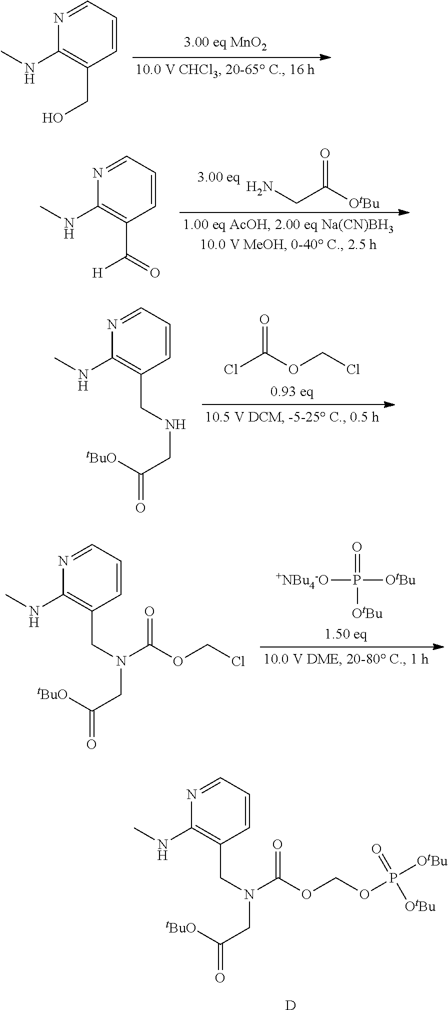

### Step 1: Synthesis of 2-(methylamino)nicotinaldehyde

To a solution of (2-(methylamino)pyridin-3-yl)methanol (24.0 g, 173 mmol) in CHCl₃ (192 mL) at room temperature under nitrogen atmosphere was added manganese dioxide (45.3 g, 521 mmol) and the resulting mixture was heated at 60° C. for 16 hours. The reaction was then cooled to room temperature and filtered, the filter cake was washed with EtOAc (3M) mL), the combined filtrate was washed with a solution of Na₂SO₃ (20.0 g) in 200 mL water. The aqueous layer was then extracted with EtOAc (2×300 mL) and the combined organic layer was dried over sodium sulfate, filtered, concentrated, and used directly in next step. LCMS-ESI+ (m/z): calcd H+ for C7H8N2O, Theoretical: 136.06, Found: 137.032.

### Step 2: Synthesis of tert-butyl ((2-(methylamino)pyridin-3-yl)methyl)glycinate

To a solution of 2-(methylamino)nicotinaldehyde (21.2 g, 155 mmol) in MeOH (630 mL) at room temperature was added tert-butyl glycinate (61.2 g, 467 mmol) followed by AcOH (9.35 g, 155 mmol). The mixture was then heated at 40° C. for 30 minutes before it was cooled to 0° C. Into this cold mixture was added NaBH₃CN (19.5 g, 311 mmol). The reaction was then removed from cooling bath and stirred at room temperature for 2 hours. The reaction mixture was concentrated, redissolved in sat. aqueous NaHCO₃ (200 mL) and extracted with DCM (3×300 mL). The combined organic layer was dried over sodium sulfate, filtered, concentrated and purified by normal phase chromatography on silica gel. LCMS-ESI+ (m/z): calcd H+ for C13H21N3O2, Theoretical: 251.16, Found: 251.971.

### Step 3: Synthesis of tert-butyl N-((chloromethoxy)carbonyl)-N-((2-(methylamino)pyridin-3-yl)methyl)glycinate

To a solution of tert-butyl ((2-(methylamino)pyridin-3-yl)methyl)glycinate (10.0 g, 39.1 mmol) in DCM (105 mL) at 0° C. was added a solution of chloromethyl carbonochloridate (4.73 g, 36.6 mmol) and stirred for 0.5 hour. The reaction was quenched with sat. NaHCO₃ (200 mL) at 0° C., and stirred for 10 minutes. The mixture was extracted with EtOAc (3×300 mL) and the combined organic layer was washed with sat. NaHCO₃ (3×200 mL), dried over sodium sulfate, filtered, concentrated and purified by normal phase chromatography on silica gel. LCMS-ESI+ (m/z): calcd H+ for C15H22ClN3O4, Theoretical: 343.13, Found: 343.950.

### Step 4: Synthesis of tert-butyl N-((((di-tert-butoxyphosphoryl)oxy)methoxy)carbonyl)-N-((2-(methylamino)pyridin-3-yl)methyl)glycinate (Intermediate D)

To a solution of tert-butyl N-((chloromethoxy)carbonyl)-N-((2-(methylamino)pyridin-3-yl)methyl)glycinate (6.0 g, 16.5 mmol) in DME (60 mL) at room temperature was added tetra-n-butylammonium di-tert-butylphosphate (11.2 g, 24.8 mmol). The reaction was heated at 80° C. for 1 hour and then cooled to room temperature. The reaction was concentrated, redissolved in 4:1 EtOAc/Hexane (50 mL), washed sequentially with brine (2×50.0 mL), with water (50.0 mL) and brine (50.0 mL), dried over sodium sulfate, filtered, concentrated and purified by normal phase chromatography on silica gel. 1H NMR (400 MHz, DMSO-d6) δ 8.01-7.94 (m, 1H), 7.31-7.14 (m, 1H), 6.55-6.45 (m, 1H), 6.10-5.86 (m, 1H), 5.54-5.46 (m, 2H), 4.31 (s, 2H), 3.96-3.88 (m, 2H), 2.82 (d, J=4.6 Hz, 3H), 1.45-1.37 (m, 18H), 1.33 (s, 9H). LCMS-ESI+ (m/z): calcd H+ for C23H40N3O8P, Theoretical: 517.26, Found: 518.30.

### Intermediate E: Synthesis of tert-butyl 5-(((2-(tert-butoxy)-2-oxoethyl((((di-tert-butoxyphosphoryl)oxy)methoxy)carbonyl)amino)methyl)-6-(methylamino)nicotinate (Intermediate E)

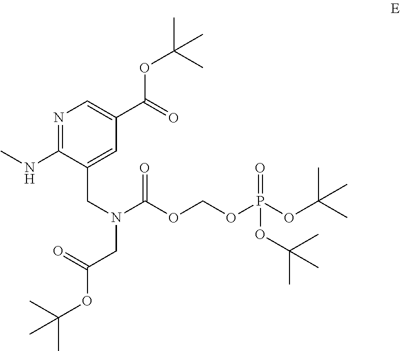 [SMILES: CNc1ncc(C(=O)OC(C)(C)C)cc1CN(CC(=O)OC(C)(C)C)C(=O)OCOP(=O)(OC(C)(C)C)OC(C)(C)C]

The title compound was prepared following similar method for tert-butyl N-((((di-tert-butoxyphosphoryl)oxy)methoxy)carbonyl)-N-((2-(methylamino)pyridin-3-yl)methyl)glycinate (Intermediate D) using tert-butyl 5-(hydroxymethyl)-6-(methylamino)nicotinate (prepared according to WO 2023102239) instead of (2-(methylamino)pyridin-3-yl)methanol in step 1. MS (m/z) 617.831 [M+H]⁺.

### Intermediate F: Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate

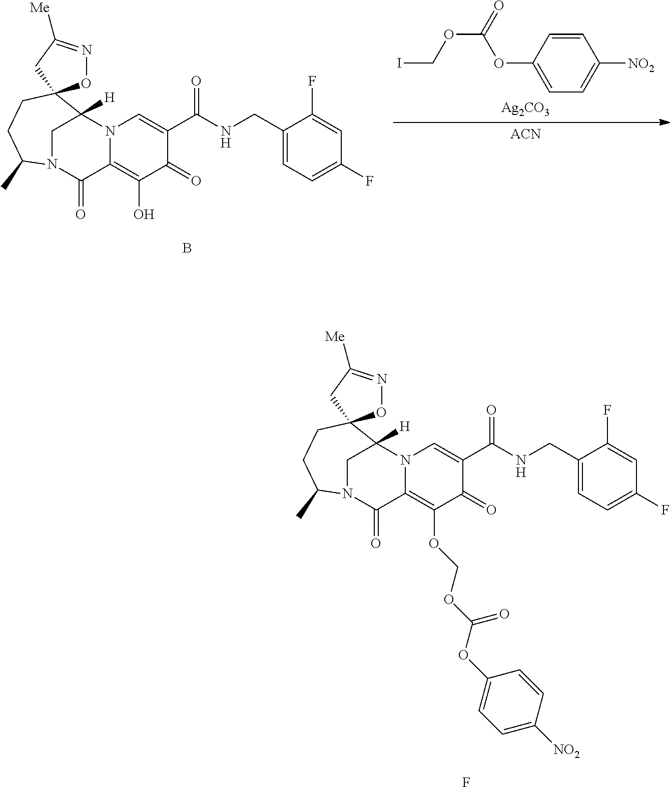

(3′S,5S,7′R)—N-(2,4-difluorobenzyl)-12′-hydroxy-3,3-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 3.06 g, 6.29 mol) and iodomethyl (4-nitrophenyl) carbonate (4.23 g, 13.1 mmol), prepared according to WO2010011812, were mixed with acetonitrile (73 mL). Ag₂CO₃ (5.29 g, 19.2 mmol) was added in one portion. The slurry was stirred at room temperature overnight. The reaction mixture was filtered through celite. The filtrate was collected and concentrated to dryness. The residue was purified on silica gel column with 0-20% MeOH in DCM to afford the crude material upon concentration. The crude product was then dissolved in EtOAc (200 ml) and was treated with water (200 mL) with agitation. Organic phase was separated and dried over Na₂SO₄ and was filtered. The filtrate was concentrated to afford Intermediate F. Calculated for C₃₂H₂₉F₂N₅O₁₀: 681.19, Found MS (ESI+): 682.01 [M+H]+.

### Example 1: Synthesis of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl methyl(3-((phosphonooxy)methyl)pyridin-2-yl)carbamate (1)

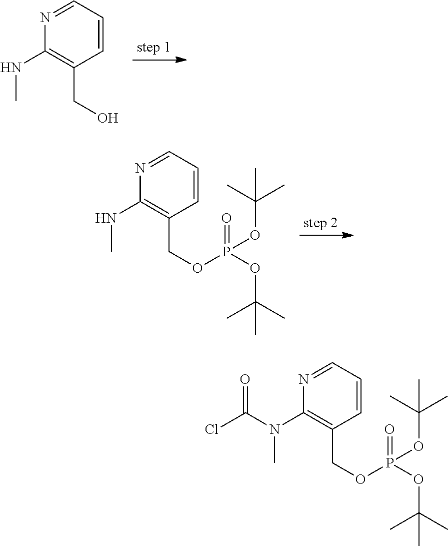

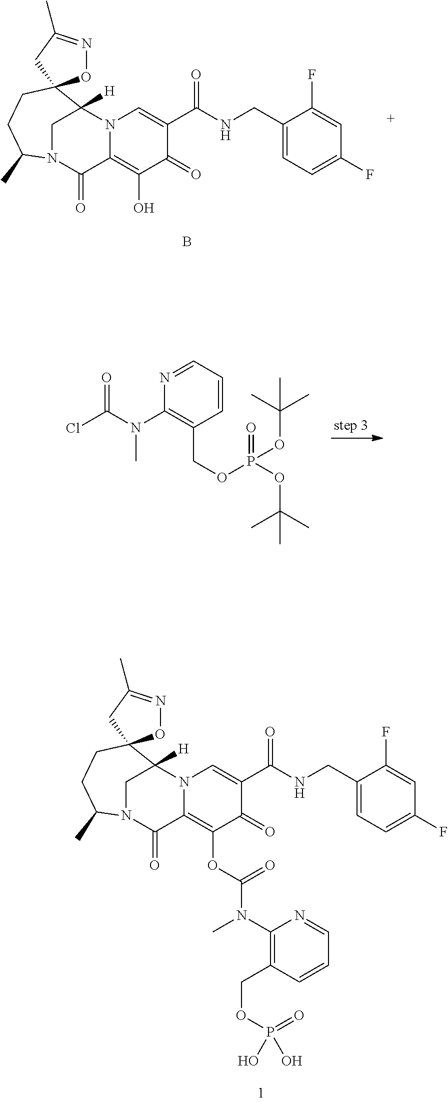

### Step 1: Synthesis of ° di-tert-butyl ((2-(methylamino)pyridin-3-yl)methyl)phosphate

To a solution of (2-(methylamino)pyridin-3-yl)methanol (2.0 g, 14.5 mmol) in DMF (50 mL) at room temperature was added imidazole (0.986 g, 14.5 mmol) and imidazole-HCl (2.28 g, 21.7 mmol) followed by N-di-tert-butoxyphosphanyl-N-isopropyl-propan-2-amine (8.02 g, 29.0 mmol). The resulting mixture was cooled to 0° C. before a solution of TBHP (3.26 g, 36.2 mmol) in decane (5-6 M) was added. The reaction was then removed from cooling bath and stirred at room temperature for one hour. The reaction was cooled back to 0° C., and quenched with IN sodium thiosulfate in water. After being stirred vigorously for 10 min, the reaction was extracted with EtOAc, organic layer was washed with brine, dried over sodium sulfate, filtered and concentrated, the residue was purified by normal phase silica gel chromatography. LCMS-ESI+ (m/z): calcd H+ for C15H27N2O4P, Theoretical: 330.17. Found: 330.846.

### Step 2: Synthesis of di-tert-butyl ((2-((chlorocarbonyl)(methylamino)pyridin-3-yl)methyl) phosphate

To a solution of di-tert-butyl ((2-(methylamino)pyridin-3-yl)methyl) phosphate (633 mg, 1.92 mmol) in THF (6.0 mL) at 0° C. was added pyridine (151 mg, 1.92 mmol) followed by bis(trichloromethyl) carbonate (569 mg, 1.92 mmol). The resulting mixture was removed from cooling bath and stirred at room temperature for two hours before it was diluted with EtOAc, washed with 1N HCl, water, brine, dried over sodium sulfate, filtered and concentrated to give title compound, which was used directly in next step. LCMS-ESI+ (m/z): calcd H+ for C16H26ClN2O5P, Theoretical: 392.13, Found: 392.588.

### Step 3: Synthesis of (3′S,5S,7′R)-10′-((2,4-difluorobenzyl)carbamoyl)-3,3′-dimethyl-1′11′-dioxo-1′,4′,5′,11′-tetrahydro-3′H,4H,7′H-spiro[isoxazole-5,6′-[2,7]methanopyrido[1,2-a][1,4]diazonin]-12′-yl methyl(3-((phosphonooxy)methyl)pyridin-2-yl)carbamate (1)

To a solution of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 550 mg, 1.13 mmol) in DCE (10.0 mL) at room temperature was added N,N-diisopropylethylamine (190 mg, 1.47 mmol) followed by di-tert-butyl ((2-((chlorocarbonyl)(methyl)amino)pyridin-3-yl)methyl) phosphate (711 mg, 1.81 mmol) and DMAP (41.4 mg, 0.339 mmol). The reaction was stirred for thirty minutes and then diluted with DCM, washed with IN HCl, water, brine, dried over sodium sulfate, filtered and concentrated. The residue was purified by normal phase silica gel chromatography. The residue obtained was dissolved in DCM (15.0 mL) at 0° C., and treated with TFA (3.75 mL) dropwise. After being stirred at 0° C. for ten minutes, the reaction was removed from cooling bath and stirred at room temperature for twenty minutes. The reaction was then concentrated, diluted with EtOAc and concentrated again. The residue was dissolved in DMF (6 mL), filtered and purified by reverse phase chromatography (5-100% MeCN/H₂O w/ 0.1% TFA) to afford the title compound. 1H NMR (400 MHz, DMSO-d6) δ 10.29-10.08 (m, 1H), 8.95-8.66 (m, 1H), 8.48 (dd, J=4.7, 1.8 Hz, 1H), 8.03-7.89 (m, 1H), 7.51-7.36 (m, 2H), 7.29-7.17 (m, 1H), 7.12-7.03 (m, 1H), 5.38-4.80 (m, 2H), 4.75-4.44 (m, 4H), 3.94-3.59 (m, 2H), 3.53-3.16 (m, 3H), 3.09-2.91 (m, 1H), 2.65 (d, J=15.5 Hz, 1H), 1.94 (s, 3H), 1.87-1.71 (m, 3H), 1.31-1.04 (m, 4H). LCMS-ESI+ (m/z): calcd H+ for C32H33F2N6O10P, Theoretical: 730.20, Found: 730.82.

### Example 2. Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl(3-((phosphonooxy)methyl)pyridin-2-yl)carbamate (2)

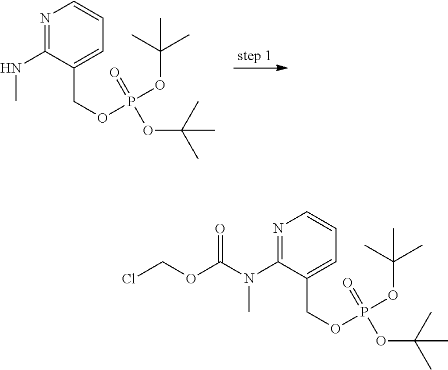 [SMILES: CNc1ncccc1COP(=O)(OC(C)(C)C)OC(C)(C)C ; CN(C(=O)OCCl)c1ncccc1COP(=O)(OC(C)(C)C)OC(C)(C)C]

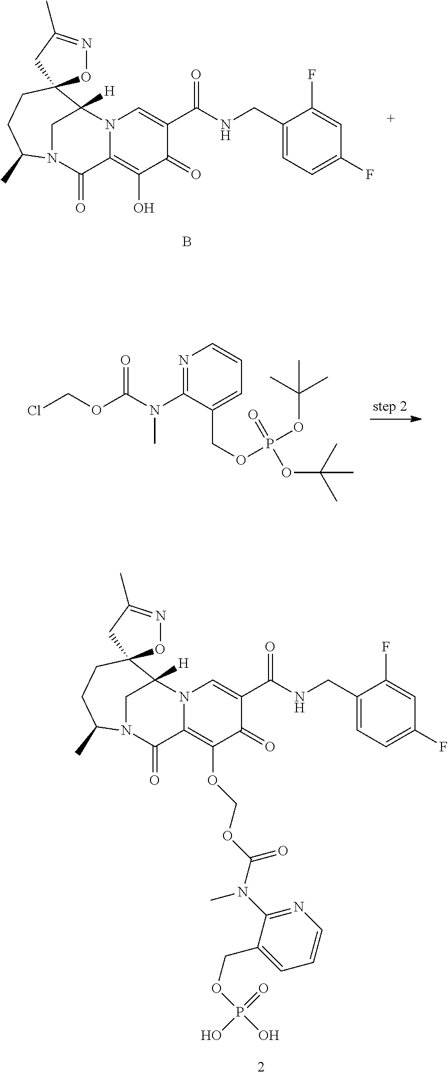

### Step 1: Synthesis of chloromethyl (3-(((di-tert-butoxyphosphoryl)oxy)methyl)pyridin-2-yl)(methyl)carbamate

To a solution of di-tert-butyl [2-(methylamino)-3-pyridyl]methyl phosphate (76 mg, 0.23 mmol) in DCM (3.0 mL) at 0° C. was added N,N-diisopropylethylamine (44.6 mg, 0.345 mmol) followed by chloromethyl carbonochloridate (38.6 mg, 0.299 mmol). The resulting mixture was then removed from cooling bath and stirred at room temperature for one hour, additional portions of chloromethyl carbonochloridate (29.6 mg, 0.23 mmol) and DIPEA (29.7 mg, 0.23 mmol) were added and stirred for one hour. The reaction was then diluted with DCM, washed with IN HCl, water, brine, dried over sodium sulfate, filtered, and concentrated. The residue was purified by normal phase silicagel chromatography. LCMS-ESI+ (m/z): calcd H+ for C17H28C1N2O6P, Theoretical: 422.14. Found: 422.527.

### Step 2: Synthesis of (3′S,5S,7′R)-10′-((2,4-difluorobenzyl)carbamoyl-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl(3-((phosphonooxy)methyl)pyridin-2-yl)carbamate (2)

To a solution of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 360 mg, 0.74 mmol) and chloromethyl (3-(((di-tert-butoxyphosphoryl)oxy)methyl)pyridin-2-yl)(methyl)carbamate (437 mg, 1.03 mmol) in DMF (6.0 mL) at room temperature was added potassium carbonate (184 mg, 1.33 mmol). The resulting mixture was heated at 50° C. for 12 hours. The reaction was then cooled to room temperature, diluted with EtOAc, washed with water, brine, dried over sodium sulfate, filtered and concentrated. The residue was purified by normal phase silica gel chromatography. The residue obtained was then dissolved in DCM (5.0 mL) at 0° C., and treated with TFA (2.0 mL) slowly. The mixture was then removed from cooling bath after addition and stirred at room temperature for 1 hr. The mixture was concentrated, dissolved in DMF, filtered, and purified by reverse phase preparative HPLC (5-100% MeCN/H₂O w/ 0.1% TFA) to afford the title compound. 1H NMR (400 MHz, Acetone-d6) S 8.60 (s, 1H), 8.47-8.39 (m, 1H), 8.02-7.95 (m, 1H), 7.49 (td, J=8.5, 6.5 Hz, 1H), 7.42-7.34 (m, 1H), 7.07-6.89 (m, 2H), 5.95-5.70 (m, 1H), 5.07 (d, J=49.0 Hz, 5H), 4.62 (d, J=5.4 Hz, 3H), 3.93 (dd, J=15.2, 2.6 Hz, 1H), 3.87-3.76 (m, 1H), 3.38-3.13 (m, 4H), 2.73 (d, J=17.7 Hz, 1H), 1.99 (s, 3H), 1.96-1.84 (m, 3H), 1.65-1.51 (m, 1H), 1.28 (d, J=6.5 Hz, 3H). LCMS-ESI+ (m/z): calcd H+ for C33H35F2N6O11 P, Theoretical: 760.21, Found: 760.923.

### Example 3: (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methylmethyl(3-((phosphonooxy)methyl)pyridin-2-yl)carbamate (3)

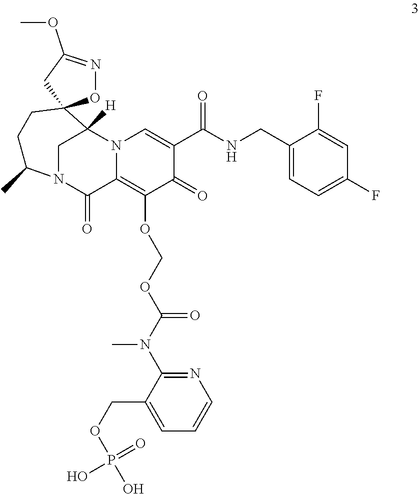 [SMILES: COC1=NO[C@@]2(CC[C@H](C)N3C[C@H]2n2cc(C(=O)NCc4ccc(F)cc4F)c(=O)c(OCOC(=O)N(C)c4ncccc4COP(=O)(O)O)c2C3=O)C1]

The title compound was synthesized following the same method for the preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl(3-((phosphonooxy)methyl)pyridin-2-yl)carbamate (Example 2, step 2) except (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B) was replaced by (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate C). 1H NMR (400 MHz, Acetone-d6) S 8.65 (s, 1H), 8.43 (dd, J=4.7, 1.8 Hz, 1H), 7.98 (dd, J=7.7, 1.8 Hz, 1H), 7.49 (td, J=8.6, 6.5 Hz, 1H), 7.38 (dd, J=7.7, 4.7 Hz, 1H), 7.07-6.85 (m, 2H), 5.95-5.70 (m, 1H), 5.22-4.81 (m, 3H), 4.76 (s, 1H), 4.62 (d, J=5.8 Hz, 2H), 3.85 (s, 7H), 3.35-3.14 (m, 4H), 2.81 (d, J=17.1 Hz, 1H), 2.03-1.86 (m, 3H), 1.70-1.58 (m, 1H), 1.28 (d, J=6.7 Hz, 3H). LCMS-ESI+ (m/z): calcd H+ for C33H35F2N6O12P, Theoretical: 776.20, Found: 776.76.

### Example 4: Synthesis of 6-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)-5-((phosphonooxy)methyl)nicotinic acid (4)

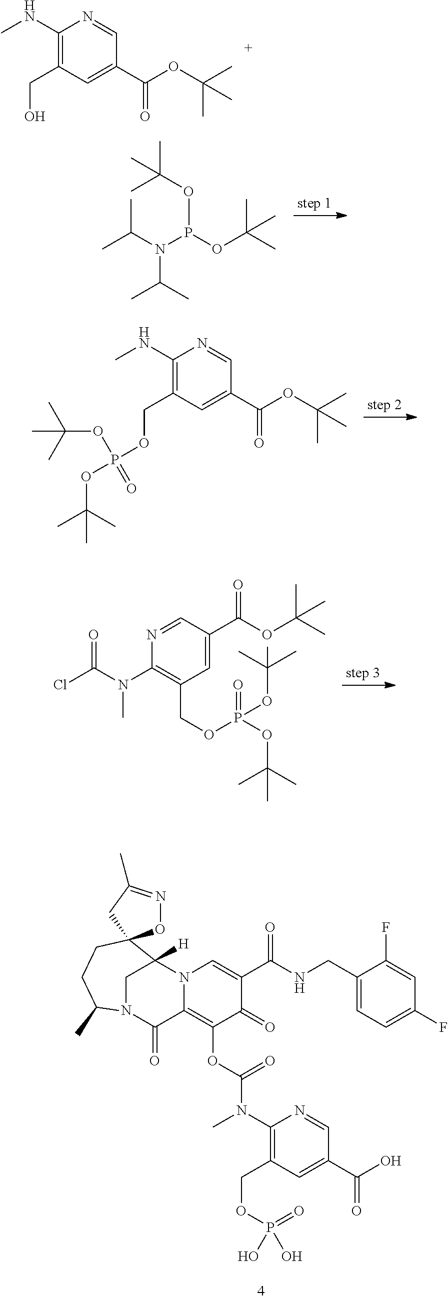

### Step 1: Synthesis of tert-butyl S-(((di-tert-butoxyphosphoryl)oxy)methyl)-6-(methylamino)nicotinate

To a solution of tert-butyl 5-(hydroxymethyl)-6-(methylamino)nicotinate (prepared according to WO 2023102239) (0.1 g, 0.42 mmol) in DMF (50 mL) at room temperature was added di-tert-butyl diisopropylphosphoramidite (349 mg, 1.26 mmol) and 1H-tetrazole (103 mg, 1.47 mmol). The resulting mixture was heated at 50° C. for 2 hours. The reaction was then cooled to 0° C. before 30 wt % hydrogen peroxide (190 mg, 1.68 mmol) was added. The resulting mixture was then removed from cooling bath and stirred at room temperature for 16 hours. The reaction was then quenched with IN sodium thiosulfate, extracted with EtOAc, organic layer was washed with brine, dried over sodium sulfate, filtered and concentrated, the residue was purified by normal phase silica gel chromatography. LCMS-ESI+ (m/z): calcd H+ for C20H35N2O6P. Theoretical: 430.22, Found: 430.917.

### Step 2: Synthesis of tert-butyl 6-((chlorocarbonyl)(methyl)amino)-5-(((di-tert-butoxyphosphoryl)oxy)methyl)nicotinate

This synthesis followed the same method for the preparation of di-tert-butyl ((2-((chlorocarbonyl)(methyl)amino)pyridin-3-yl)methyl) phosphate (Example 1, step 2) except di-tert-butyl ((2-(methylamino)pyridin-3-yl)methyl) phosphate was replaced by tert-butyl 5-(((di-tert-butoxyphosphoryl)oxy)methyl)-6-(methylamino)nicotinate. The product was used directly in next step.

### Step 3: Synthesis of 6-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)-5-((phosphonooxy)methyl)nicotinic acid

This synthesis followed the same method for the preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl methyl(3-((phosphonooxy)methyl)pyridin-2-yl)carbamate (Example 1, step 3) except di-tert-butyl ((2-((chlorocarbonyl)(methyl)amino)pyridin-3-yl)methyl) phosphate was replaced by tert-butyl 6-((chlorocarbonyl)(methyl)amino)-5-(((di-tert-butoxyphosphoryl)oxy)methyl)nicotinate. 1H NMR (400 MHz, Acetone-d6) S 10.23 (s, 1H), 9.01 (d, J=2.0 Hz, 1H), 8.73-8.62 (m, 1H), 8.60-8.52 (m, 1H), 7.49 (q, J=9.0, 8.4 Hz, 1H), 7.07-6.90 (m, 2H), 5.41-5.13 (m, 1H), 4.89-4.71 (n, 1H), 4.69-4.52 (m, 3H), 3.9-3.8 (m, 3H), 3.65-3.40 (m, 3H), 3.21 (d, J=17.6 Hz, 11H), 2.73 (d, J=18.3 Hz, 11H), 2.00 (s, 3H), 1.96-1.85 (m, 3H), 1.64-1.47 (m, 11H), 1.26 (d, J=6.5 Hz, 3H). LCMS-ESI+ (m/z): calcd H+ for C33H33F2N6O12P, Theoretical: 774.19, Found: 774.805.

### Example 5 and Example 6: Synthesis of tert-butyl N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)-N-(((phosphonooxy)methoxy)carbonyl)glycinate (5) and N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)-N-(((phosphonooxy)methoxy)carbonyl)glycine (6)

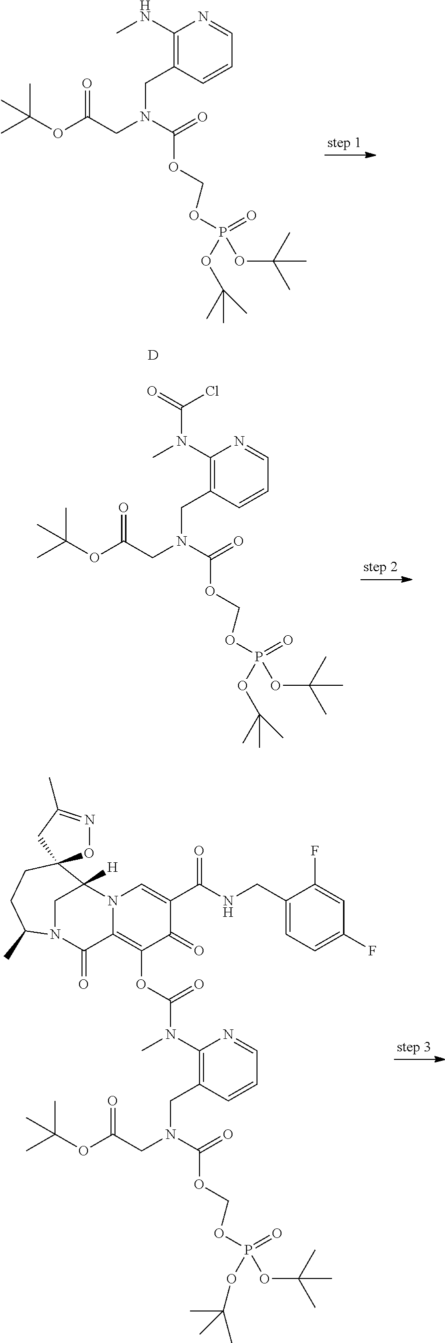

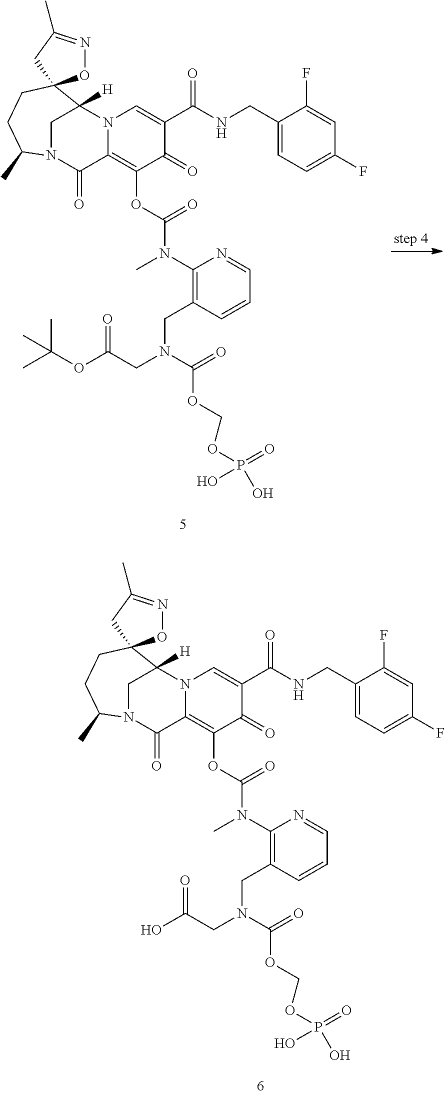 [SMILES: CC1=NO[C@@]2(CC[C@H](C)N3C[C@H]2n2cc(C(=O)NCc4ccc(F)cc4F)c(=O)c(OC(=O)N(C)c4ncccc4CN(CC(=O)OC(C)(C)C)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(C)c4ncccc4CN(CC(=O)O)C(=O)OCOP(=O)(O)O)c2C3=O)C1]

### Step 1. Synthesis of tert-butyl N-((2-((chlorocarbonyl)methyl)amino)pyridin-3-yl)methyl)-N-((((di-tert-butoxyphosphoryl)oxy)methoxycarbonyl)glycinate

Followed the same method for the preparation of di-tert-butyl ((2-((chlorocarbonyl)(methyl)amino)pyridin-3-yl)methyl) phosphate (Example 1, step 2) using tert-butyl N-((((di-tert-butoxyphosphoryl)oxy)methoxy)carbonyl)-N-((2-(methylamino)pyridin-3-yl)methyl)glycinate (Intermediate D) instead of di-tert-butyl ((2-(methylamino)pyridin-3-yl)methyl) phosphate. MS (m/z): [M+Na]+601.91.

### Step 2: Synthesis of tert-butyl N-((((di-tert-butoxyphosphoryl)oxy)methoxy)carbonyl)-N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)glycinate

To a mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 700 mg, 1.44 mmol) and tert-butyl N-((2-((chlorocarbonyl)(methyl)amino)pyridin-3-yl)methyl)-N-((((di-tert-butoxyphosphoryl)oxy)methoxy)carbonyl)glycinate (1.335 g, 2.30 mmol) in DMF (7.0 mL) at room temperature was added triethylamine (437 mg, 4.32 mmol) and DMAP (105 mg, 0.863 mmol). The reaction was stirred for 2 hours before it was diluted with EtOAc, washed with IN HCl, water, brine, dried over sodium sulfate, filtered and concentrated, purified by normal phase silica gel chromatography. MS (m/z): 1029.36 [M]⁺

### Step 3: Synthesis of tert-butyl N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-ylmethyl)-N-(((phosphonooxy)methoxy)carbonyl)glycinate (5)

Tert-butyl N-((((di-tert-butoxyphosphoryl)oxy)methoxy)carbonyl)-N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)glycinate (50 mg, 0.0485 mmol) was dissolved in DCM (1 mL) at 0° C., and TFA (0.2 mL) was added. After 30 minutes, the reaction was concentrated, redissolved in EtOAc and concentrated again. The residue was dissolved in DMF, filtered and purified by reverse phase preparative HPLC. 1H NMR (400 MHz, Methanol-d4) δ 8.70-8.57 (m, 1H), 8.53-8.41 (m, 1H), 8.20-7.96 (m, 1H), 7.60-7.39 (m, 2H), 6.97 (q, J=10.2, 9.1 Hz, 2H), 5.75-5.42 (m, 2H), 5.15-4.37 (m, 6H), 4.31-3.95 (m, 2H), 3.91-3.68 (m, 2H), 3.64-3.47 (m, 1H), 3.42-3.34 (m, 2H), 3.22-3.07 (m, 1H), 2.69 (dd, J=17.4, 6.9 Hz, 1H), 2.05 (d, J=3.2 Hz, 3H), 2.01-1.83 (m, 3H), 1.42 (d, J=17.1 Hz, 10H), 1.24 (d, J=16.3 Hz, 3H). LCMS-ESI+ (m/z): calcd H+ for C40H46F2N7O14P, Theoretical: 917.28, Found: 917.86.

### Step 4: Synthesis of N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl(methyl)amino)pyridin-3-yl)methyl)-N-(((phosphonooxy)methoxycarbonyl)glycine (6)

Tert-butyl N-((((di-tert-butoxyphosphoryl)oxy)methoxy)carbonyl)-N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)glycinate (1.143 g, 1.11 mmol) was dissolved in DCM (12 mL) at 0° C., and TFA (3.0 mL) was added. The resulting mixture was then removed from cooling bath and stirred at room temperature for 3 hours. The reaction was concentrated, redissolved in EtOAc and concentrated again. The residue was dissolved in DMF, filtered and purified by reverse phase preparative HPLC (5-100% MeCN/H₂O w/ 0.1% TFA). 1H NMR (400 MHz, Methanol-d4) δ 8.72-8.55 (m, 1H), 8.45 (s, 1H), 8.13-7.96 (m, 1H), 7.55-7.37 (m, 2H), 7.04-6.90 (m, 2H), 5.73-5.45 (m, 2H), 5.15-4.00 (m, 7H), 3.92-3.67 (m, 2H), 3.66-3.47 (m, 1H), 3.37 (s, 3H), 3.22-3.06 (m, 1H), 2.76-2.59 (m, 1H), 2.05 (d, J=5.6 Hz, 3H), 2.01-1.79 (m, 3H), 1.67-1.40 (m, 1H), 1.35-1.16 (m, 3H). LCMS-ESI+ (m/z): calcd H+ for C36H38F2N7O14P, Theoretical: 861.22, Found: 861.843.

### Example 7: Synthesis of N-((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)-pyridin-3-yl)methyl)-N-(((phosphonooxy)methoxy)carbonyl)glycine (7)

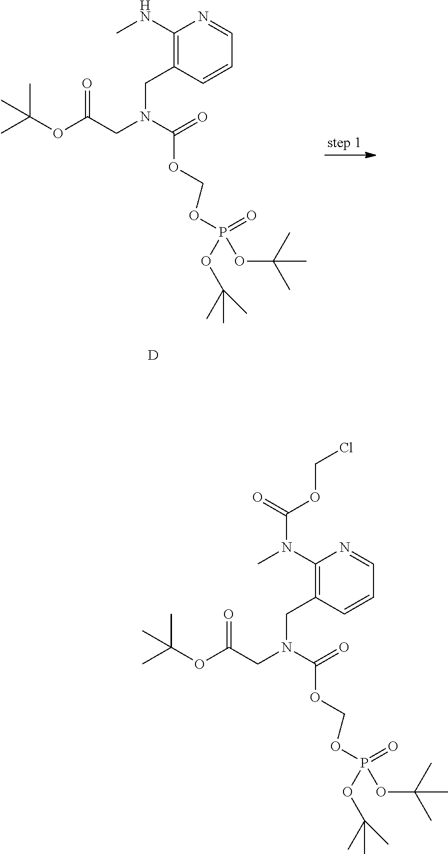

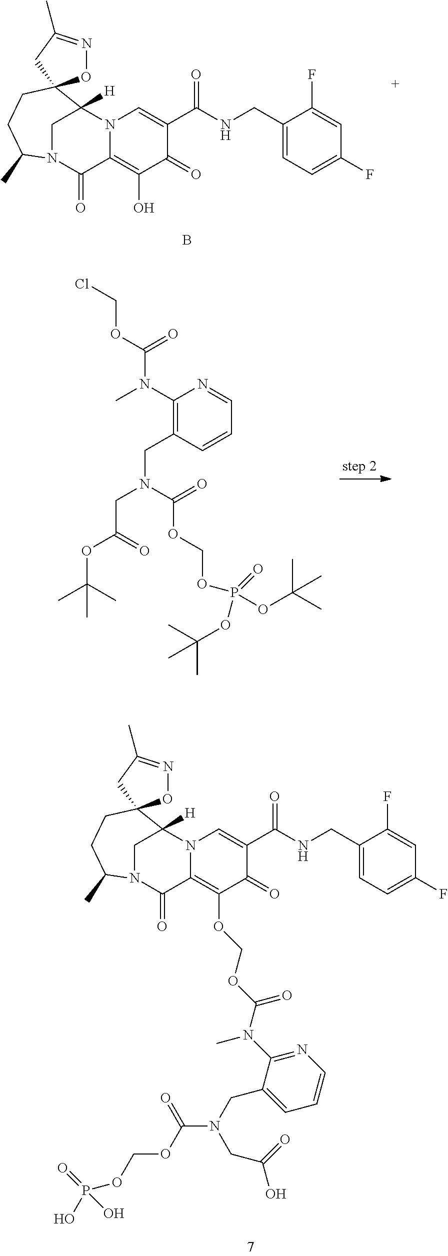

### Step 1: Synthesis of tert-butyl N-((2-(((chloromethoxy)carbonyl)(methyl)amino)pyridin-3-yl)methyl)-N-((((di-tert-butoxyhosphoryl)oxy)methoxy)carbonyli)glycinate

To a solution of tert-butyl N-((((di-tert-butoxyphosphoryl)oxy)methoxy)carbonyl)-N-((2-(methylamino)pyridin-3-yl)methyl)glycinate (Intermediate D) (1135 mg, 2.19 mmol) in DCM (20.0 mL) at 0° C. was added N,N-diisopropylethylamine (567 mg, 4.39 mmol) followed by chloromethyl carbonochloridate (509 mg, 3.95 mmol). The resulting mixture was stirred at 0° C. for 20 minutes and then at room temperature for 20 minutes. Additional chloromethyl carbonochloridate (141.4 mg, 1.1 mmol) and DIPEA (141.75 mg, 1.1 mmol) were added and stirred for 30 minutes. The reaction was then diluted with DCM, washed with IN HCl, water, brine, dried over sodium sulfate, filtered and concentrated. The residue was purified by normal phase silica gel chromatography. MS (m/z): 631.87 [M+Na]⁺.

### Step 2: Synthesis of N-((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)-pyridin-3-yl)methyl)-N-(((phosphonooxy)methoxy)carbonyl)glycine (7)

To a solution of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 650 mg, 1.34 mmol) and tert-butyl N-((2-(((chloromethoxy)carbonyl) (methyl)amino)pyridin-3-yl)methyl)-N-((((di-tert-butoxyphosphoryl)oxy)methoxy) carbonyl)-glycinate (1060 mg, 1.74 mmol) in DMF (7.0 mL) at room temperature was added potassium carbonate (369 mg, 2.67 mmol) and tetrabutylammonium iodide (494 mg, 1.34 mmol). The resulting mixture was heated at 55° C. for four hours. The reaction was then cooled to room temperature, diluted with EtOAc, washed with water and brine, dried over sodium sulfate, filtered, and concentrated. The residue was purified by normal phase silica gel chromatography. The obtained residue was then dissolved DCM (12 mL) at 0° C., treated with TFA (4.0 mL). The mixture was stirred at 0° C. for 20 minutes and then at room temperature for 4 hours. Then it was concentrated, dissolved in DMF, filtered and purified by reverse phase preparative HPLC (5-100% MeCN/H₂O w/ 0.1% TFA). ¹H NMR (400 MHz, Methanol-d4) δ 8.54 (s, 1H), 8.44-8.33 (m, 1H), 8.05-7.88 (m, 1H), 7.52-7.35 (m, 2H), 7.05-6.87 (m, 2H), 6.03-5.49 (m, 4H), 4.87-4.40 (m, 6H), 4.20-3.94 (m, 2H), 3.90-3.73 (m, 2H), 3.32-3.09 (m, 4H), 2.79-2.59 (m, 1H), 2.06 (d, J=2.8 Hz, 3H), 2.00-1.83 (m, 3H), 1.62-1.49 (m, 1H), 1.28 (d, J=6.6 Hz, 3H). LCMS-ESI+ (m/z): calcd H+ for C₃₋₇H40F2N7O15P, Theoretical: 891.23, Found: 891.856.

### Example 8: Synthesis of 5-(((carboxymethyl)(((phosphonooxy)methoxy)carbonyl)amino) methyl)-6-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)nicotinic acid (8)

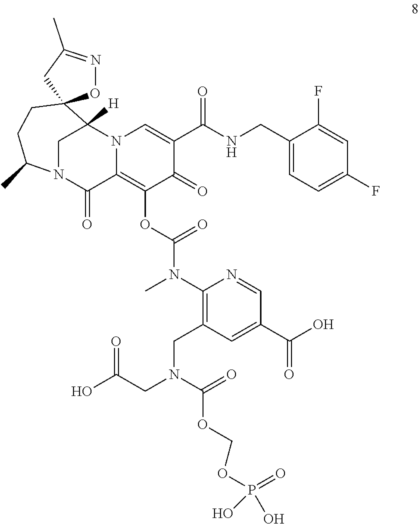 [SMILES: CC1=NO[C@@]2(CC[C@H](C)N3C[C@H]2n2cc(C(=O)NCc4ccc(F)cc4F)c(=O)c(OC(=O)N(C)c4ncc(C(=O)O)cc4CN(CC(=O)O)C(=O)OCOP(=O)(O)O)c2C3=O)C1]

The title compound was synthesized following the similar method for the preparation of N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)-N-(((phosphonooxy)methoxy)carbonyl)glycine (Example 6, Steps 1, 2 and 4) except in step 1 using tert-butyl 5-(((2-(tert-butoxy)-2-oxoethyl)((((di-tert-butoxyphosphoryl)oxy)methoxy) carbonyl)amino)methyl)-6-(methylamino)nicotinate (Intermediate E) to replace tert-butyl N-((((di-tert-butoxyphosphoryl)oxy)methoxy)carbonyl)-N-((2-(methylamino)pyridin-3-yl)methyl)glycinate (Intermediate D) and using DCE instead of DMF in step 4. 1 H NMR (400 MHz, Methanol-d4) δ 8.99 (d, J=2.1 Hz, 1H), 8.74-8.39 (m, 2H), 7.51-7.38 (m, 1H), 7.05-6.87 (m, 2H), 5.73-5.39 (m, 2H), 5.18-4.09 (m, 9H), 3.92-3.50 (m, 3H), 3.47-3.36 (m, 1H), 3.22-3.04 (m, 1H), 2.78-2.58 (m, 1H), 2.05 (d, J=2.6 Hz, 3H), 2.00-1.77 (m, 3H), 1.68-1.43 (m, 1H), 1.36-1.13 (m, 3H). LCMS-ESI+ (m/z): calcd H+ for C₃₋₇H38F2N7O16P, Theoretical: 905.21, Found: 905.942.

### Example 9: Synthesis of (2-((((((3′S,5S,7′R)-10′-((2,4-difluorobenzyl)carbamoyl)-3,3′-dimethyl-1′11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino) pyridin-3-yl)methyl methylglycinate (9)

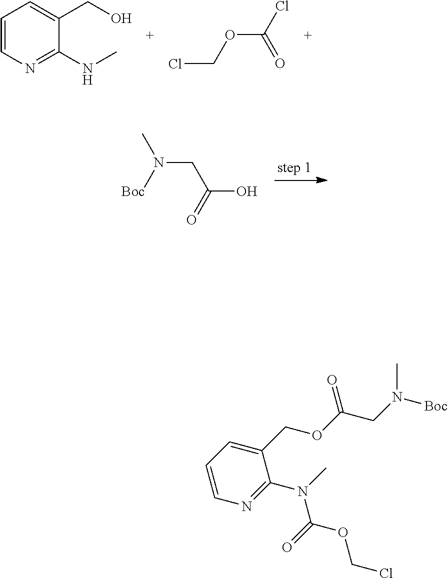

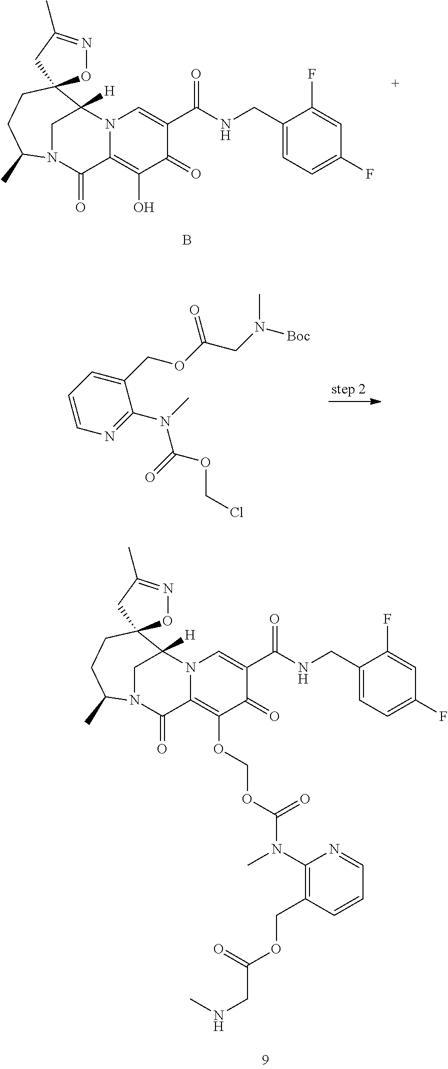

### Step 1: Synthesis of (Z-(((chloromethoxy)carbonyl)(methyl)amino)pyridin-3-yl)methyl N-(tert-butoxycarbonyl)-N-methylglycinate

To a solution of [2-(methylamino)-3-pyridyl]methanol (100 mg, 0.724 mmol) in DCM (4.0 mL) at 0° C. was added N,N-diisopropylethylamine (112 mg, 0.869 mmol) followed by chloromethyl carbonochloridate (103 mg, 0.796 mmol) dropwise. The resulting mixture was stirred at 0° C. for 30 min before 2-[tert-butoxycarbonyl(methyl)amino]acetic acid (137 mg, 0.724 mmol) was added. 1-Ethyl-3-(3-dimethylaminopropyl)carbodiimide hydrochloride (138 mg, 0.724 mmol) and DMAP (88.3 mg, 0.724 mmol) were added afterwards. After stirring for 30 minutes, the reaction was diluted with DCM, washed with water and brine, dried over sodium sulfate, filtered, concentrated and purified by normal phase silica gel chromatography. LCMS-ESI+ (m/z): calcd H+ for C17H24C1N3O6, Theoretical: 401.14, Found: 401.698.

### Step 2: Synthesis of (2-((((((3′S,5S,7′R)-10′-((2,4-difluorobenzyl)carbamoyl-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methylamino)pyridin-3-yl)methyl methylglycinate (9)

Followed the similar method for the preparation of N-((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)-N-(((phosphonooxy)methoxy)carbonyl)glycine (Example 7, step 2) except tert-butyl N-((2-(((chloromethoxy)carbonyl)(methyl)amino)pyridin-3-yl)methyl)-N-((((di-tert-butoxyphosphoryl)oxy)methoxy)carbonyl)glycinate was replaced by (2-(((chloromethoxy)carbonyl)(methyl)amino)pyridin-3-yl)methyl N-(tert-butoxycarbonyl)-N-methylglycinate. 1H NMR (400 MHz, DMSO-d6) δ 10.30 (s, 1H), 8.93 (s, 2H), 8.68 (s, 1H), 8.52-8.42 (m, 1H), 7.92 (dd, J=7.7, 1.8 Hz, 1H), 7.49-7.38 (m, 2H), 7.25 (t, J=9.9 Hz, 1H), 7.07 (t, J=8.5 Hz, 1H), 5.98-5.63 (m, 1H), 5.35-5.14 (m, 2H), 4.74-4.61 (m, 2H), 4.61-4.48 (m, 2H), 4.07 (s, 2H), 3.70 (s, 2H), 3.27-3.09 (m, 3H), 2.99 (d, J=17.6 Hz, 1H), 2.65-2.56 (m, 4H), 1.95 (s, 3H), 1.88-1.68 (m, 3H), 1.40-1.24 (m, 1H), 1.19 (d, J=6.6 Hz, 3H). LCMS-ESI+ (m/z): calcd H+ for C36H39F2N7O9. Theoretical: 751.28, Found: 752.097.

### Example 10: (2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl L-lysinate (10)

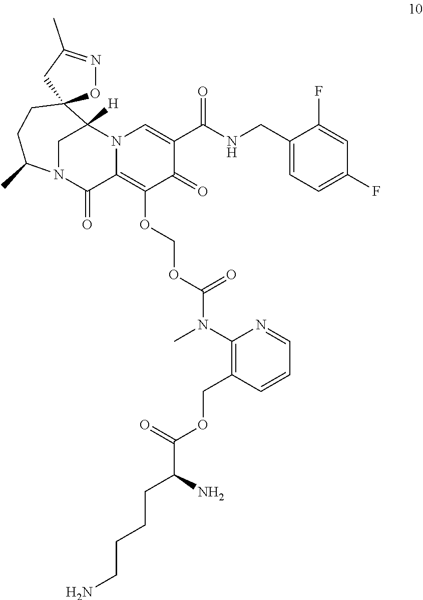 [SMILES: CC1=NO[C@@]2(CC[C@H](C)N3C[C@H]2n2cc(C(=O)NCc4ccc(F)cc4F)c(=O)c(OCOC(=O)N(C)c4ncccc4COC(=O)[C@@H](N)CCCCN)c2C3=O)C1]

The title compound was synthesized following the same method for the preparation of (2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl methylglycinate (Example 9) except in step 1 using N2,N6-bis(tert-butoxycarbonyl)-L-lysine instead of 2-[tert-butoxycarbonyl(methyl)amino]acetic acid. 1H NMR (400 MHz, Methanol-d4) δ 10.62-10.37 (m, 1H), 8.60 (s, 1H), 8.54-8.43 (m, 1H), 8.06-7.97 (m, 1H), 7.51-7.41 (m, 2H), 7.05-6.89 (m, 2H), 6.21-5.12 (m, 3H), 4.94-4.8 (m, 2H), 4.66 (q, J=15.1 Hz, 2H), 4.58-4.45 (m, 1H), 4.24-4.06 (m, 1H), 3.92-3.74 (m, 2H), 3.32-3.10 (m, 4H), 2.94 (t, J=7.7 Hz, 2H), 2.68 (d, J=18.0 Hz, 1H), 2.10-1.86 (m, 8H), 1.78-1.65 (m, 2H), 1.63-1.46 (m, 3H), 1.29 (d, J=6.8 Hz, 3H). LCMS-ESI+ (m/z): calcd H+ for C39H46F2N809, Theoretical: 808.34, Found: 809.167.

### Example 11: (2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl L-alaninate (11)

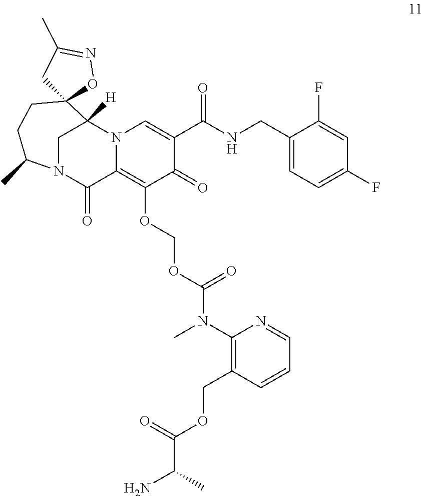 [SMILES: CC1=NO[C@@]2(CC[C@H](C)N3C[C@H]2n2cc(C(=O)NCc4ccc(F)cc4F)c(=O)c(OCOC(=O)N(C)c4ncccc4COC(=O)[C@H](C)N)c2C3=O)C1]

The title compound was synthesized following the same method for the preparation of (2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl methylglycinate (Example 9) except in step 1 using (tert-butoxycarbonyl)-L-alanine instead of 2-[tert-butoxycarbonyl(methyl)amino]acetic acid. 1H NMR (400 MHz, Methanol-d4) δ 8.57 (s, 1H), 8.53-8.41 (m, 1H), 8.03-7.92 (m, 1H), 7.52-7.41 (m, 2H), 7.05-6.88 (m, 2H), 6.02-5.38 (m, 1H), 5.34-5.19 (m, 1H), 4.76-4.59 (m, 2H), 4.48 (s, 1H), 4.23-4.12 (m, 1H), 3.93-3.74 (m, 2H), 3.32-3.04 (m, 7H), 2.75-2.61 (m, 1H), 2.06 (s, 3H), 2.02-1.85 (m, 3H), 1.55 (d, J=7.4 Hz, 4H), 1.29 (d, J=6.5 Hz, 3H). LCMS-ESI+ (m/z): calcd H+ for C36H39F2N7O9, Theoretical; 751.28, Found: 752.001.

### Example 12: (2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl L-valinate (12)

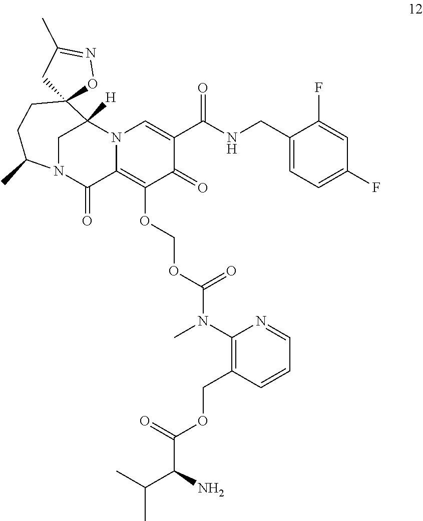 [SMILES: CC1=NO[C@@]2(CC[C@H](C)N3C[C@H]2n2cc(C(=O)NCc4ccc(F)cc4F)c(=O)c(OCOC(=O)N(C)c4ncccc4COC(=O)[C@@H](N)C(C)C)c2C3=O)C1]

The title compound was synthesized following the same method for the preparation of (2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl methylglycinate (Example 9) except in step 1 using by (tert-butoxycarbonyl)-D-valine instead of 2-[tert-butoxycarbonyl(methyl)amino]acetic acid. 1H NMR (400 MHz, Methanol-d4) δ 8.58 (s, 1H), 8.48 (s, 1H), 8.07-7.94 (m, 1H), 7.53-7.40 (m, 2H), 7.05-6.90 (m, 2H), 6.04-5.70 (m, 1H), 5.60-5.16 (m, 1H), 4.78-4.86 (m, 1H), 4.74-4.57 (m, 2H), 4.56-4.43 (m, 1H), 3.96 (s, 1H), 3.90-3.74 (m, 2H), 3.32-3.09 (m, 6H), 2.76-2.60 (nm, 1H), 2.37-2.22 (m, 1H), 2.06 (s, 3H), 2.02-1.85 (m, 3H), 1.62-1.46 (m, 1H), 1.29 (d, J=6.9 Hz, 3H), 1.16-0.98 (m, 6H). LCMS-ESI+(m/z): calcd H+ for C38H43F2N7O9, Theoretical: 779.31, Found: 780.055.

### Example 13 and 14: Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl(((S)-pyrrolidin-2-yl)methyl)carbamate (13) and (phosphonooxy)methyl (2S)-2-(((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)methyl)pyrrolidine-1-carboxylate (14)

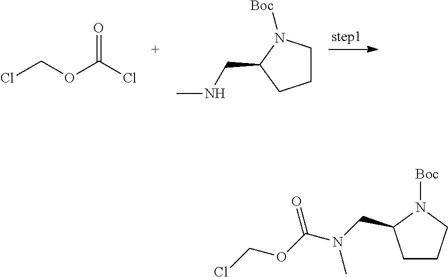 [SMILES: O=C(Cl)OCCl ; CNC[C@@H]1CCCN1C(=O)OC(C)(C)C ; CN(C[C@@H]1CCCN1C(=O)OC(C)(C)C)C(=O)OCCl]

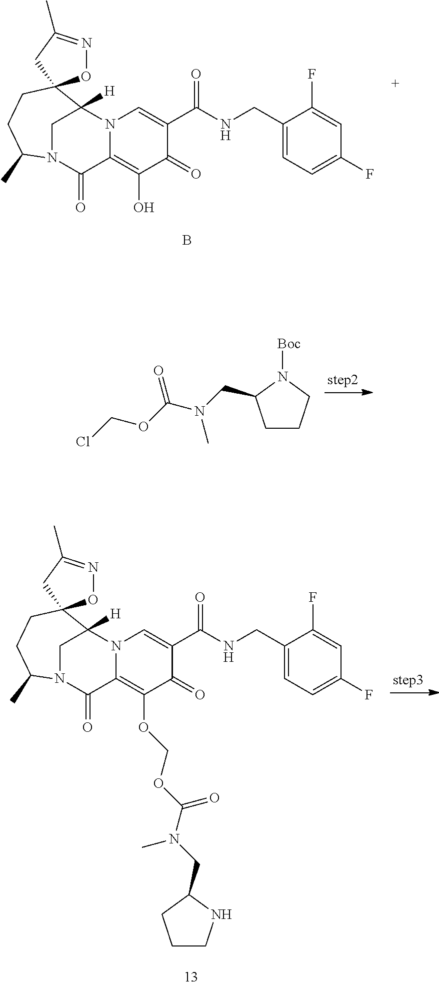

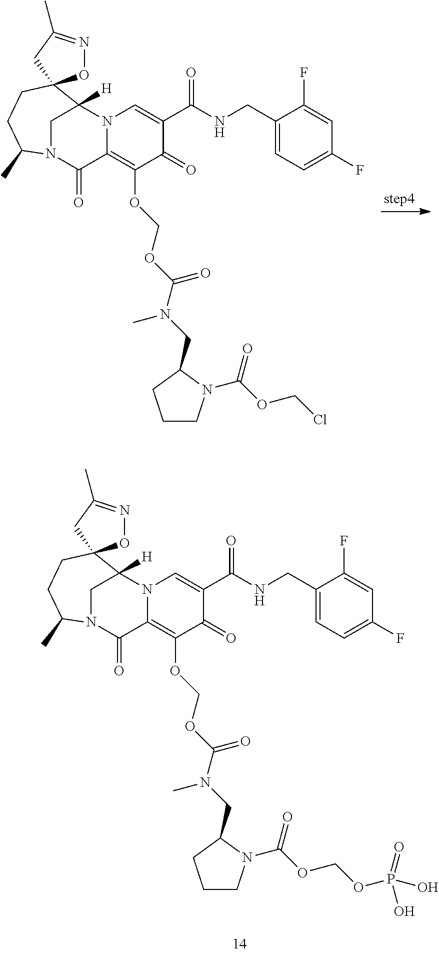 [SMILES: CC1=NO[C@@]2(CC[C@H](C)N3C[C@H]2n2cc(C(=O)NCc4ccc(F)cc4F)c(=O)c(OCOC(=O)N(C)C[C@@H]4CCCN4C(=O)OCCl)c2C3=O)C1 ; CC1=NO[C@@]2(CC[C@H](C)N3C[C@H]2n2cc(C(=O)NCc4ccc(F)cc4F)c(=O)c(OCOC(=O)N(C)C[C@@H]4CCCN4C(=O)OCOP(=O)(O)O)c2C3=O)C1]

### Step 1: Synthesis of tert-butyl (S)-2-((((chloromethoxy)carbonyl)(methyl)amino)methylpyrrolidine-1-carboxylate

To a solution of tert-butyl (S)-2-((methylamino)methyl)pyrrolidine-1-carboxylate (1000 mg, 4.67 mmol) in DCM (60.0 mL) at 0° C. was added N,N-diisopropylethylamine (154.0 mg, 1.19 mmol) followed by chloromethyl carbonochloridate (1327 mg, 10.3 mmol). The resulting mixture was stirred at 0° C. for 30 minutes and then room temperature for 30 minutes. The reaction was then diluted with DCM, washed with IN HCl, water and brine, dried over sodium sulfate, filtered, concentrated and purified by normal phase silica gel chromatography. MS (m/z): [M+H-Boc]⁺ 207.06.

### Step 2: Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl(((S)-pyrrolidin-2-yl)methyl)carbamate (13)

Followed the same method for the preparation of N-((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)-N-(((phosphonooxy)methoxy)carbonyl)glycine (Example 7, step 2) except tert-butyl N-((2-(((chloromethoxy)carbonyl)(methyl)amino)pyridin-3-yl)methyl)-N-((((di-tert-butoxyphosphoryl)oxy)methoxy)carbonyl)glycinate was replaced by tert-butyl (S)-2-((((chloromethoxy)carbonyl)(methyl)amino)methyl)pyrrolidine-1-carboxylate. Title compound obtained as bis TFA salt after reverse phase preparative HPLC. 1H NMR (400 MHz, Methanol-d4) δ 8.69-8.56 (m, 1H), 7.47 (td, J=8.6, 6.3 Hz, 1H), 7.10-6.92 (m, 2H), 5.96-5.88 (m, 1H), 5.72-5.63 (m, 1H), 4.88-4.76 (m, 1H), 4.76-4.46 (m, 3H), 4.00-3.71 (m, 4H), 3.55-3.36 (m, 2H), 3.31-3.23 (m, 2H), 3.16 (d, J=18.1 Hz, 1H), 3.01-2.86 (m, 3H), 2.78-2.65 (m, 1H), 2.32-2.10 (m, 2H), 2.08-2.05 (m, 3H), 2.03-1.88 (m, 3H), 1.84-1.69 (m, 1H), 1.65-1.50 (m, 1H), 1.28 (d, J=6.7 Hz, 3H). LCMS-ESI+ (m/z): calcd H+ for C32H38F2N6O7, Theoretical: 656.28, Found: 657.154.

### Step 3: Synthesis of chloromethyl (2S)-2-(((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)methyl)pyrrolidine-1-carboxylate

To a solution of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl(((S)-pyrrolidin-2-yl)methyl)carbamate bis TFA salt from step 2 (772 mg, 0.873 mmol) in DCM (60.0 mL) at 0° C. was added triethylamine (265.0 mg, 2.62 mmol) followed by chloromethyl carbonochloridate (169 mg, 1.31 mmol). The resulting mixture was stirred at 0° C. for 30 minutes before it was diluted with DCM, washed with IN HCl, water, and brine, dried over sodium sulfate, filtered, concentrated and purified by normal phase silica gel chromatography. LCMS-ESI+ (m/z): calcd H+ for C34H39ClF2N6O9, Theoretical: 748.24. Found: 748.772.

### Step 4: Synthesis of (phosphonooxy)methyl (2S)-2-(((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)methyl)pyrrolidine-1-carboxylate (14)

To a solution of chloromethyl (2S)-2-(((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)methyl)pyrrolidine-1-carboxylate (821.0 mg, 1.10 mmol) in DME (5.5 mL) at room temperature was added tetra-n-butylammonium di-tert-butylphosphate (695 mg, 1.53 mmol) and tetrabutylammonium iodide (405 mg, 1.10 mmol). The resulting mixture was heated at 60° C. for one hour. The reaction was then cooled to room temperature, diluted with EtOAc, washed with water and brine, dried over sodium sulfate, filtered, concentrated, and purified by normal phase silica gel chromatography. The obtained residue was then dissolved in DCM (1.2 mL) at 0° C., and treated with TFA (0.3 mL) for 1 hour. The reaction was then concentrated, dissolved in DMF, filtered and purified by reverse phase preparative HPLC (5-100% MeCN/H₂O w/ 0.1% TFA). 1H NMR (400 MHz, Methanol-d4) δ 8.57 (d, J=3.1 Hz, 1H), 7.46 (q, J=7.9 Hz, 1H), 6.98 (dt, J=14.5, 9.2 Hz, 2H), 5.88-5.70 (m, 2H), 5.67-5.49 (m, 2H), 4.87-4.77 (m, 1H), 4.71-4.57 (m, 2H), 4.50 (s, 1H), 4.27-4.00 (m, 1H), 3.89-3.70 (m, 2H), 3.51-3.26 (m, 5H), 3.16 (d, J=17.9 Hz, 1H), 2.97 (d, J=7.6 Hz, 1H), 2.90 (d, J=4.5 Hz, 1H), 2.69 (dd, J=18.4, 5.5 Hz, 1H), 2.06 (s, 3H), 2.00-1.75 (m, 7H), 1.61-1.49 (m, 1H), 1.26 (d, J=6.7 Hz, 3H). LCMS-ESI+ (m/z): calcd H+ for C34H41F2N6O13P, Theoretical: 810.24, Found: 810.83.

### Example 15: (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl(((S)-pyrrolidin-2-yl)methyl)carbamate (15)

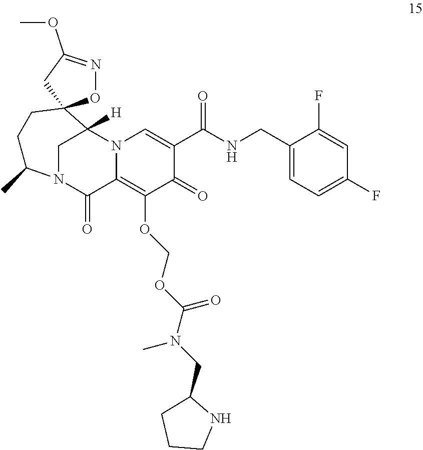 [SMILES: COC1=NO[C@@]2(CC[C@H](C)N3C[C@H]2n2cc(C(=O)NCc4ccc(F)cc4F)c(=O)c(OCOC(=O)N(C)C[C@@H]4CCCN4)c2C3=O)C1]

The title compound was synthesized following the same method for the preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl(((S)-pyrrolidin-2-yl)methyl)carbamate (Example 13) except in step 2, (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide was replaced by (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide. 1H NMR (400 MHz, Methanol-d4) δ 10.51-10.21 (m, 1H), 8.72-8.62 (m, 1H), 7.52-7.41 (m, 1H), 7.10-6.92 (m, 2H), 5.92 (d, J=6.8 Hz, 1H), 5.68 (dd, J=9.1, 6.6 Hz, 1H), 4.85-4.57 (m, 4H), 4.00-3.89 (m, 4H), 3.89-3.71 (m, 3H), 3.55-3.35 (m, 2H), 3.31-3.23 (m, 1H), 3.18 (dd, J=17.1, 4.8 Hz, 1H), 3.02-2.87 (m, 3H), 2.78 (dd, J=17.1, 6.5 Hz, 1H), 2.31-1.90 (m, 6H), 1.83-1.49 (m, 2H), 1.28 (d, J=6.7 Hz, 3H). LCMS-ESI+ (m/z): calcd H+ for C32H38F2N6O8. Theoretical: 672.27, Found: 673.119.

### Example 16: Synthesis of (phosphonooxy)methyl (2S)-2-(((((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)methyl)pyrrolidine-1-carboxylate (16)

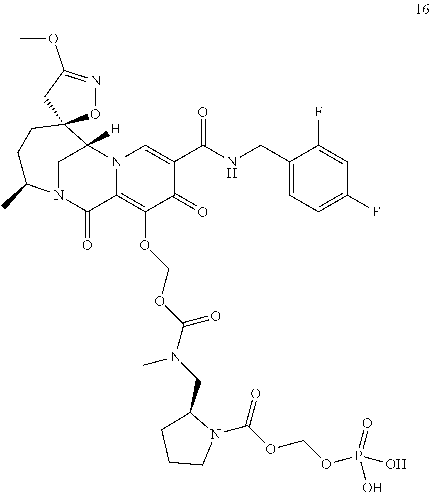 [SMILES: COC1=NO[C@@]2(CC[C@H](C)N3C[C@H]2n2cc(C(=O)NCc4ccc(F)cc4F)c(=O)c(OCOC(=O)N(C)C[C@@H]4CCCN4C(=O)OCOP(=O)(O)O)c2C3=O)C1]

The title compound was synthesized following the same method for the preparation of (phosphonooxy)methyl (2S)-2-(((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)methyl)pyrrolidine-1-carboxylate (Example 14) except (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide was replaced by (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide. 1H NMR (400 MHz, Methanol-d4) δ 8.62 (d, J=2.2 Hz, 1H), 7.46 (q, J=8.2 Hz, 1H), 6.98 (dt, J=14.2, 9.5 Hz, 2H), 5.87-5.70 (m, 2H), 5.66-5.48 (m, 2H), 4.76-4.83 (m, 1H), 4.67-4.58 (m, 3H), 4.28-4.00 (m, 1H), 3.90 (s, 3H), 3.87-3.70 (m, 2H), 3.48-3.28 (m, 5H), 3.18 (d, J=17.1 Hz, 1H), 2.97 (d, J=7.4 Hz, 1H), 2.90 (d, J=4.7 Hz, 1H), 2.76 (dd, J=17.1, 4.2 Hz, 1H), 2.10-1.76 (m, 7H), 1.61-1.50 (m, 1H), 1.26 (d, J=6.7 Hz, 3H). LCMS-ESI+ (m/z): calcd H+ for C34H41F2N6O14P, Theoretical: 826.24, Found: 826.802.

### Example 17: Synthesis of 2-((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) 1-((phosphonooxy)methyl) (2S)-pyrrolidine-1,2-dicarboxylate (17)

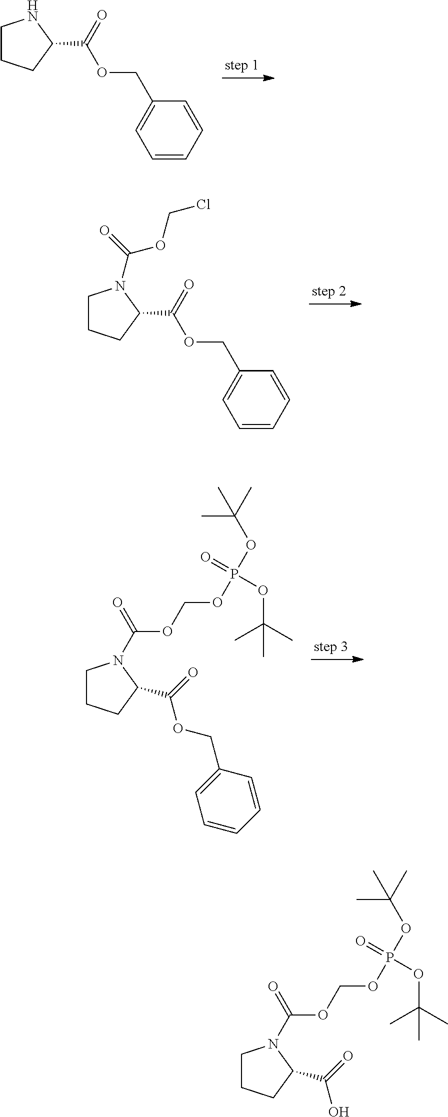 [SMILES: O=C(OCc1ccccc1)[C@@H]1CCCN1 ; O=C(OCc1ccccc1)[C@@H]1CCCN1C(=O)OCCl ; CC(C)(C)OP(=O)(OCOC(=O)N1CCC[C@H]1C(=O)OCc1ccccc1)OC(C)(C)C ; CC(C)(C)OP(=O)(OCOC(=O)N1CCC[C@H]1C(=O)O)OC(C)(C)C]

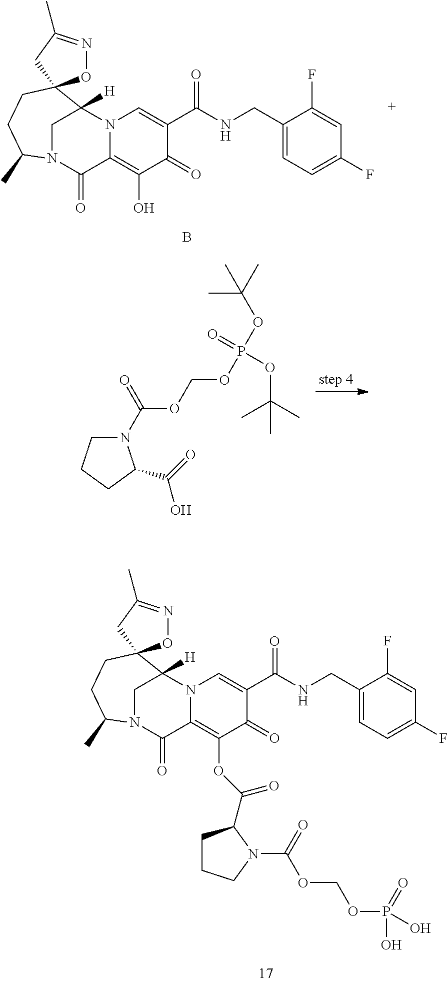

### Step 1: Synthesis of 2-benzyl 1-chloromethyl) (S))-pyrrolidine-1,2-dicarboxylate

Followed the same method for the preparation of tert-butyl (S)-2-((((chloromethoxy)carbonyl)(methyl)amino)methyl)pyrrolidine-1-carboxylate (Example 13, step 1) except tert-butyl (S)-2-((methylamino)methyl)pyrrolidine-1-carboxylate was replaced by benzyl L-prolinate and the reaction was kept at 0° C. for 10 minutes before quenching. Used directly in next step.

### Step 2: Synthesis of 2-benzyl 1-(((di-tert-butoxyphosphoryl)oxy)methyl) (S)-pyrrolidine-1,2-dicarboxylate

To a solution of 2-benzyl 1-(chloromethyl) (S)-pyrrolidine-1,2-dicarboxylate (290 mg, 0.974 mmol) in DME (10 mL) at room temperature was added tetra-n-butylammonium di-tert-butylphosphate (617 mg, 1.36 mmol) and tetrabutylammonium iodide (360 mg, 0.974 mmol). The resulting mixture was heated at 60° C. for one hour. The reaction was then cooled to room temperature, diluted with EtOAc, washed with water and brine, dried over sodium sulfate, filtered, concentrated and purified by normal phase silica gel chromatography. MS (m/z): 493.93 [M+Na]⁺.

### Step 3: Synthesis of ((((di-tert-butoxyphosphoryl)oxy)methoxy)carbonyl)-L-proline

The solution of 2-benzyl 1-(((di-tert-butoxyphosphoryl)oxy)methyl) (S)-pyrrolidine-1,2-dicarboxylate (236 mg, 0.501 mmol) in THF (10.0 mL) at room temperature was treated with 10% Pd/C (23.0 mg). The resulting mixture was degassed and flushed with nitrogen three times and then degassed and flushed with hydrogen three times before it was hydrogenated under hydrogen balloon for 1.5 hours. The reaction was then degassed and flushed with nitrogen, filtered, concentrated, and used directly in next step.

### Step 4: Synthesis of 2-((3′S,5S,7′R)-10′-((2,4-difluorobenzyl)carbamoyl)-3,3′-dimethyl-1′,11′-dioxo-1′,4′,5S′11′-tetrahydro-3′H,4H,7′H-spiro[isoxazole-5,6′-[2,7]methanopyrido[1,2-a][1,4]diazonin]-12′-yl) I-((phosphonooxy)methyl) (2S)-pyrrolidine-1,2-dicarboxylate (17)

To a mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (40 mg, 0.082 mmol) and ((((di-tert-butoxyphosphoryl)oxy)methoxy)carbonyl)-L-proline (40.8 mg, 0.107 mmol) in DMF (0.5 mL) at room temperature was added 1-ethyl-3-(3-dimethylaminopropyl)carbodiimide hydrochloride (20.4 mg, 0.107 mmol) and DMAP (13.0 mg, 0.107 mmol), followed by triethylamine (16.6 mg, 0.164 mmol). After stirring for 1 hour, the reaction was diluted with EtOAc, washed with 0.5 N HCl, water and brine, dried over sodium sulfate, filtered, concentrated, and purified by normal phase silica gel chromatography. The obtained residue was then dissolved in DCM (2.0 mL) at 0° C., and treated with TFA (0.03 mL). The reaction was removed from cooling bath after TFA was added and stirred at room temperature for 1.5 hour. The reaction was then concentrated, dissolved in DMF, filtered, and purified by reverse phase preparative HPLC (5-100% MeCN/H₂O w/ 0.1% TFA). 1H NMR (400 MHz, Methanol-d4) δ 8.66 (d, J=2.8 Hz, 1H), 7.50-7.39 (m, 1H), 7.03-6.91 (m, 2H), 5.84-5.64 (m, 1H), 5.57-5.43 (m, 1H), 4.87-4.72 (m, 2H), 4.70-4.59 (m, 2H), 4.51 (s, 1H), 3.92-3.75 (m, 2H), 3.67-3.48 (m, 2H), 3.16 (d, J=17.6 Hz, 1H), 2.70 (dd, J=17.9, 1.2 Hz, 1H), 2.66-2.56 (m, 1H), 2.47-2.27 (m, 1H), 2.18-1.85 (m, 8H), 1.63-1.48 (m, 1H), 1.26 (dd, J=6.7, 2.2 Hz, 3H). LCMS-ESI+ (m/z): calcd H+ for C31H34F2N5012P, Theoretical: 737.19, Found: 737.609.

### Example 18: Synthesis of benzyl ((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) hydrogen phosphate (18)

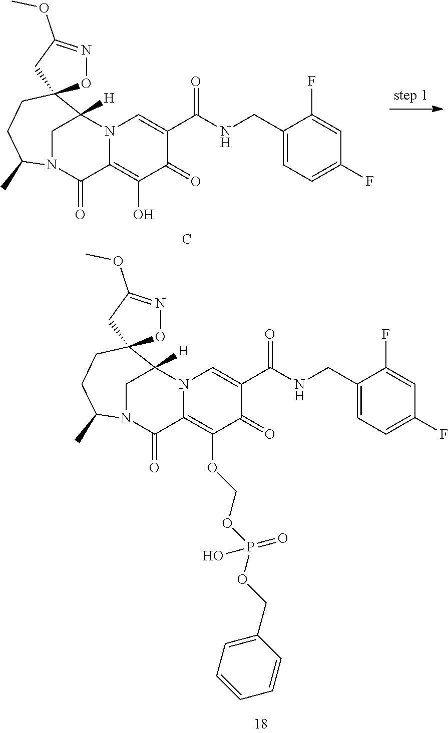

To a solution of (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate C, 30 mg, 0.0597 mmol) in DMF (0.5 mL) at room temperature was added tetrabutylammonium iodide (14.7 mg, 0.0398 mmol) and potassium carbonate (11.0 mg, 0.0796 mmol). The resulting mixture was heated at 63° C. Dibenzyl chloromethyl phosphate (19.5 mg, 0.0597 mmol) was added to the hot mixture and the newly formed mixture was stirred for 30 minutes. The reaction was then cooled to room temperature and filtered, and the filtrate was purified by reverse phase preparative chromatography (5-100% MeCN/H₂O w/ 0.1% TFA). 1H NMR (400 MHz, DMSO-d6) S 10.27 (t, J=6.0 Hz, 1H), 8.76 (s, 1H), 7.47-7.38 (m, 1H), 7.38-7.29 (m, 4H), 7.24 (ddd, J=10.5, 9.3, 2.6 Hz, 1H), 7.06 (tdd, J=8.6, 2.6, 1.0 Hz, 1H), 5.64 (dd, J=12.1, 5.1 Hz, 1H), 5.53 (dd, J=13.1, 5.0 Hz, 1H), 4.92 (d, J=7.3 Hz, 2H), 4.74 (d, J=2.1 Hz, 1H), 4.69-4.59 (m, 1H), 4.55 (d, J=5.9 Hz, 2H), 3.82 (s, 3H), 3.74-3.61 (m, 2H), 3.21-3.13 (m, 1H), 3.06 (d, J=16.9 Hz, 1H), 2.73 (d, J=17.0 Hz, 1H), 1.92-1.71 (m, 3H), 1.33-1.28 (m, 1H), 1.10 (d, J=6.7 Hz, 3H). LCMS-ESI+ (m/z): calcd H+ for C32H33F2N4010P, Theoretical: 702.19, Found: 702.722.

### Example 19: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-(phosphonooxy)benzyl) carbonate

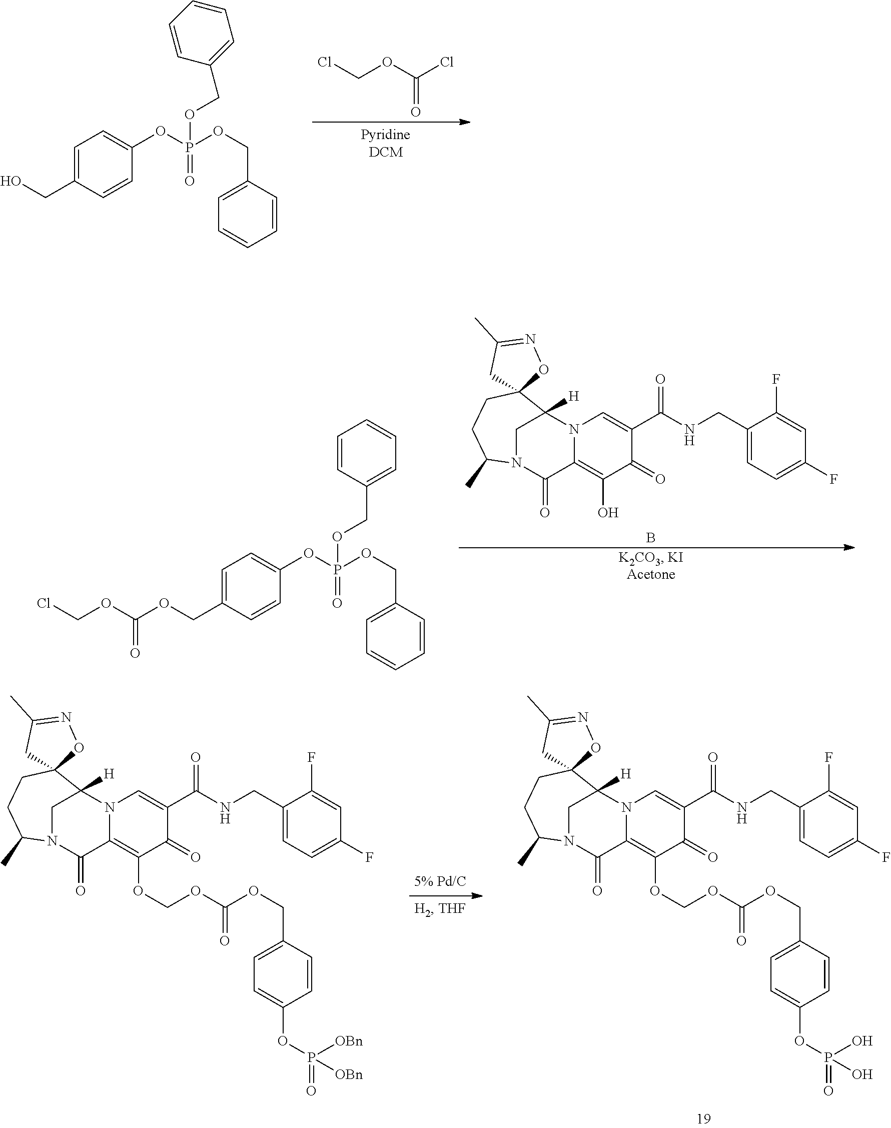

### Step 1: 4-((bis(benzyloxy)phosphoryl)oxy)benzyl (chloromethyl) carbonate

Into the mixture of dibenzyl (4-(hydroxymethyl)phenyl) phosphate (prepared according to Chem. Commun. 2020, 56, 8456-8459) (760 mg, 1.98 mmol) and chloromethyl carbonochloridate (382 mg, 2.97 mmol) in DCM (30 mL) was added pyridine (235 mg, 2.97 mmol) at ice-bath cooling. After warming to rt for 2 h, the reaction mixture was extracted with EtOAc (50 ml). The organic layer was washed with brine, dried with MgSO₄, and concentrated. The residue was purified by column chromatography on silica gel to afford the title compound. MS (m/z) 477.569 [M]⁺.

### Step 2: 4-((bis(benzyloxy)phosphoryl)oxy)benzyl ((((3′S,5S,7′R)-10′-(2,4-difluorobenzyl)carbamoyl)-3,3′-<1methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

Into the mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 200 mg, 0.411 mmol) and chloromethyl (4-dibenzyloxyphosphoryloxyphenyl)methyl carbonate (235 mg, 0.493 mmol) in acetone (20 mL) was added K₂CO₃ (114 mg, 0.822 mmol) and KI (81.9 mg, 0.493 mmol) at rt. After 3 days at rt, the reaction mixture was extracted with EtOAc (100 ml) and washed with brine. After drying with MgSO₄, the solvent was removed and the resulting residue was purified by column chromatography on silica gel to afford the title compound. MS (m/z) 926.938 [M+H].

### Step 3: (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-(phosphonooxy)benzyl) carbonate (19)

Into the solution of 4-((bis(benzyloxy)phosphoryl)oxy)benzyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (59 mg, 0.0637 mmol) in THF (12 mL) was added 5% palladium on carbon (13.5 mg), then the reaction mixture was stirred under H2 atmosphere for 1 h. The reaction mixture was filtered through Celite® and washed with DCM. Then the solution was concentrated to obtain title compound. MS (m/z) 746.956 [M+H]⁺. ¹H NMR (400 MHz, DMSO-d6) δ 10.28 (t, J=6.0 Hz, 1H), 8.70 (s, 1H), 7.42 (d, J=7.7 Hz, 1H), 7.33 (d, J=8.1 Hz, 2H), 7.23 (d, J=8.7 Hz, 1H), 7.15 (d, J=8.2 Hz, 2H), 7.07 (d, J=2.4 Hz, 1H), 5.85 (d, J=6.5 Hz, 1H), 5.66 (d, J=6.5 Hz, 1H), 5.07 (t, J=10.7 Hz, 2H), 4.62 (s, 2H), 4.55 (t, J=6.9 Hz, 2H), 3.67 (s, 2H), 2.99 (d, J=17.5 Hz, 1H), 2.62 (d, J=17.4 Hz, 1H), 1.94 (s, 3H), 1.84-1.68 (m, 3H), 1.26 (s, 1H), 1.12 (d, J=6.7 Hz, 3H).

### Example 20: Preparation of 2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetic acid

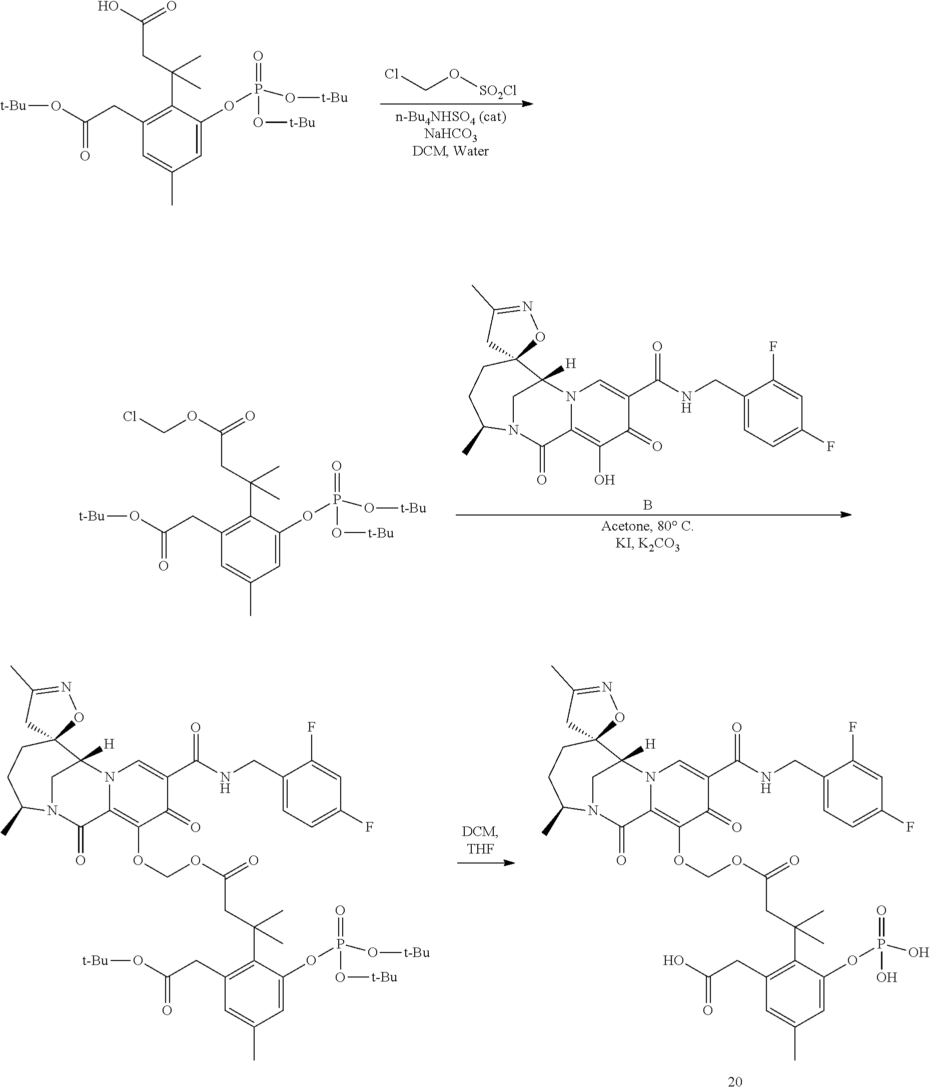

### Step 1: Chloromethyl 3-(2-(2-(tert-butoxy)-2-oxo-ethyl)-6-((di-tert-butoxyphosphoryl)oxy)-4-methylphenyl)-3-methylbutanoate

Into the mixture of 3-(2-(2-(tert-butoxy)-2-oxoethyl)-6-((di-tert-butoxyphosphoryl)oxy)-4-methylphenyl)-3-methylbutanoic acid (prepared according to WO2023102239) (275 mg, 0.534 mmol), NaHCO₃ (130 mg, 2.14 mmol) in 1:2 DCM/Water (6 mL) was added tetra-n-butyl ammonium hydrogen sulfate (18.1 mg, 0.0534 mmol). Then, chloro(chlorosulfonyloxy)methane (106 mg, 0.641 mmol) was added under vigorous stirring at ice-bath cooling. The reaction was allowed to warm to rt after addition. After stirring for 4 h at rt, the reaction mixture was extracted with EtOAc (50 ml) and the organic layer was washed with brine, dried with MgSO₄, and concentrated. The residue was purified by column chromatography on silica gel to afford the title compound. 1H NMR (400 MHz, Chloroform-d) δ 7.36 (dt, J=1.9, 0.9 Hz, 1H), 6.71 (d, J=2.0 Hz, 1H), 5.59 (s, 2H), 3.86 (s, 2H), 3.09 (s, 2H), 2.26 (s, 3H), 1.62 (s, 6H), 1.56-1.42 (m, 27H).

### Step 2: (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3-(2-(2-(tert-butoxy)-2-oxoethyl)-6-((di-tert-butoxyphosphoryl)oxy)-4-methylphenyl)-3-methylbutanoate

Into the solution of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 150 mg, 0.208 mmol) in acetone (30 ml), was added chloromethyl 3-(2-(2-(tert-butoxy)-2-oxoethyl)-6-((di-tert-butoxyphosphoryl)oxy)-4-methylphenyl)-3-methylbutanoate (208 mg, 0.37 mmol), K₂CO₃ (85.2 mg, 0.617 mmol), and KI (61.4 mg, 0.37 mmol). Then, the reaction mixture was heated to 80° C. for 16 h. The reaction mixture was extracted with EtOAc (100 ml) and the organic layer was washed with brine, dried with MgSO₄, and concentrated. The residue was purified by column chromatography on silica gel to afford the title compound. MS (m/z) 1012.795 [M+H].

### Step 3: 2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetic acid (20)

Into the solution of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3-(2-(2-(tert-butoxy)-2-oxoethyl)-6-((di-tert-butoxyphosphoryl)oxy)-4-methylphenyl)-3-methylbutanoate (55 mg, 0.0651 mmol) in DCM (3 mL) was added TFA (0.7 mL) at rt. After 4 h, the solvent and TFA were removed and the title product was precipitated from ethyl ether. The precipitated solid was collected by filtration, washed with ethyl ether, and dried to afford the title compound. MS (m/z) 845.806 [M+H]⁺. 1H NMR (400 MHz, DMSO-d6) δ 10.29 (t, J=6.0 Hz, 1H), 8.68 (s, 1H), 7.42 (td, J=8.6, 6.5 Hz, 1H), 7.24 (td, J=9.9, 2.6 Hz, 1H), 7.16-6.98 (m, 2H), 6.65 (d, J=2.0 Hz, 1H), 5.70 (d, J=6.3 Hz, 1H), 5.46 (d, J=6.3 Hz, 1H), 4.79-4.40 (m, 4H), 3.90-3.74 (m, 2H), 3.71-3.59 (m, 2H), 3.01 (dd, J=17.2, 11.7 Hz, 2H), 2.88 (d, J=16.9 Hz, 1H), 2.62 (d, J=17.6 Hz, 1H), 2.16 (s, 3H), 1.95 (s, 3H), 1.85-1.70 (m, 3H), 1.45 (d, J=4.7 Hz, 6H), 1.30 (q, J=4.2, 3.0 Hz, 1H), 1.15 (d, J=6.6 Hz, 3H).

### Example 21: Preparation of 2-(2-(4-((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetic acid

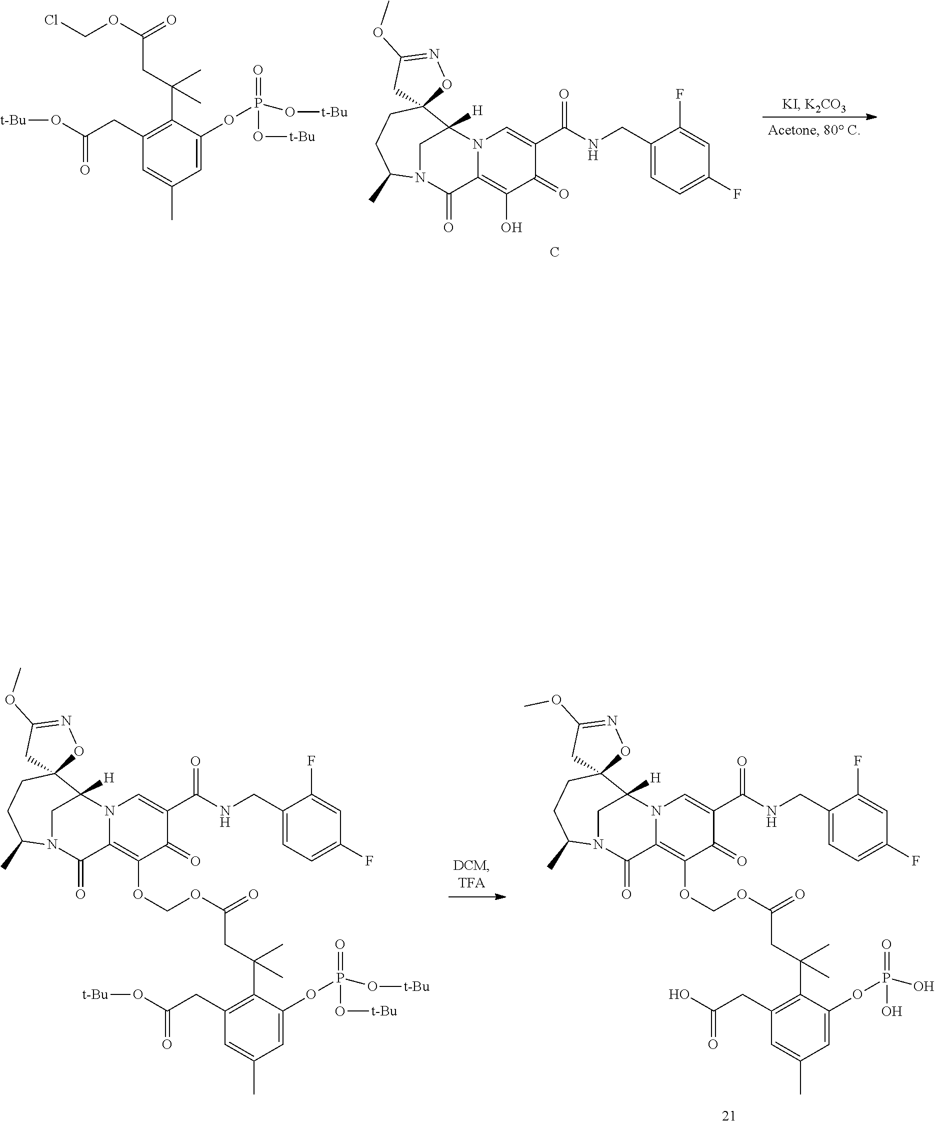

### Step 1: (((3′S,5S,7′R)-10′-((2,4-difluorobenzyl)carbamoyl)-3-methoxy-3′-methyl-1′,11′-dioxo-1′,4′,5′,11′-tetrahydro-3′H,4H7′H-spiro[isoxazole-5,6′-[2,7]methanopyrido[1,2-a][1,4]diazonin]-12′-yl)oxy)methyl 3-(2-(2-(tert-butoxy)-2-oxoethyl)-6-((di-tert-butoxyphosphoryl)oxy)-4-melhylphenyl)-3-methylbutanoate

The title compound was synthesized by the same procedure as Example 20 step 2, except starting from (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate C, 362 mg, 0.719 mmol) instead of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B). MS (m/z) 1029.896 [M+H]⁺.

### Step 2: 2-(2-(4-((((3′S,5S,7′R)-10′-((2,4-difluorobenzyl)carbamoyl)-3-methoxy-3′-methyl-1′,11′-dioxo-11′,4′,5′,11′-tetrahydro-3′H,4H,7′H-spiro[isoxazole-5,6′-[2,7]methanopyrido[1,2-a][1,4]diazonin]-12′-yl)oxy)methoxy-2-methyl-4-oxobutan-2-yl)-5-methyl-3-(phosphonooxy)phenyl)acetic acid (21)

The title compound was synthesized by the same procedure as Example 20 step 3, except using (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3-(2-(2-(tert-butoxy)-2-oxoethyl)-6-((di-tert-butoxyphosphoryl)oxy)-4-methylphenyl)-3-methylbutanoate instead of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3-(2-(2-(tert-butoxy)-2-oxoethyl)-6-((di-tert-butoxyphosphoryl)oxy)-4-methylphenyl)-3-methylbutanoate. MS (m/z) 860.91 [M+H]⁺. 1 H NMR (400 MHz, DMSO-d6) δ 10.29 (t, J=5.9 Hz, 1H), 8.72 (s, 1H), 7.41 (td, J=8.7, 6.6 Hz, 1H), 7.26 (d, J=2.6 Hz, 1H), 7.17-7.01 (m, 2H), 6.65 (d, J=2.0 Hz, 1H), 5.67 (d, J=6.2 Hz, 1H), 5.46 (d, J=6.2 Hz, 1H), 4.70 (d, J=2.2 Hz, 1H), 4.69-4.59 (m, 1H), 4.54 (t, J=5.0 Hz, 2H), 3.82 (d, J=4.4 Hz, 5H), 3.66 (d, J=2.3 Hz, 2H), 3.03 (dd, J=16.8, 13.6 Hz, 2H), 2.89 (d, J=16.7 Hz, 1H), 2.74 (d, J=16.9 Hz, 1H), 2.16 (s, 3H), 1.95-1.71 (m, 3H), 1.53-1.39 (m, 6H), 1.34 (dd, J=15.1, 11.3 Hz, 1H), 1.15 (d, J=6.7 Hz, 3H).

### Example 22: Preparation of (3′S,5S,7′R)—N-(2,4-difluorobenzyl)-3,3′-dimethyl-12′-((5-methyl-2-oxo-1,3-dioxol-4-yl)methoxy)-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (22)

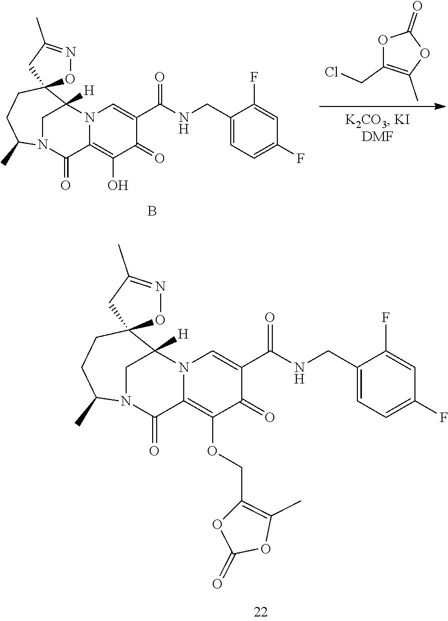

To a mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.050 g, 0.103 mmol) in DMF (1 mL) was added potassium carbonate (0.071 g, 0.514 mmol), potassium iodide (0.017 g, 0.103 mmol), and 4-(chloromethyl)-5-methyl-1,3-dioxol-2-one (0.061 g, 0.411 mmol). The mixture was stirred at room temperature overnight and filtered, rinsing the filter cake with MeCN. The filtrate was purified by reverse phase prep HPLC (5-100% MeCN/H₂O w/ 0.1% TFA) and lyophilized to afford the title compound. MS (m/z) 598.67 [M+H]+. 1H NMR (400 MHz, DMSO-d6) δ 10.32 (t, J=6.0 Hz, 1H), 8.70 (s, 1H), 7.42 (td, J=8.7, 6.6 Hz, 1H), 7.25 (ddd, J=10.5, 9.3, 2.6 Hz, 1H), 7.07 (td, J=8.6, 2.6 Hz, 1H), 5.09 (d, J=13.1 Hz, 1H), 4.92 (d, J=13.1 Hz, 1H), 4.71-4.60 (m, 2H), 4.60-4.48 (m, 2H), 3.79-3.57 (m, 2H), 3.01 (d, J=17.5 Hz, 1H), 2.62 (d, J=17.5 Hz, 1H), 2.11 (s, 3H), 1.94 (s, 3H), 1.87-1.67 (m, 3H), 1.29 (dd, J=15.2, 11.0 Hz, 1H), 1.15 (d, J=6.7 Hz, 3H).

### Example 23: Preparation 2-(2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methyl-3-(phosphonooxy)phenyl)acetic acid (23)

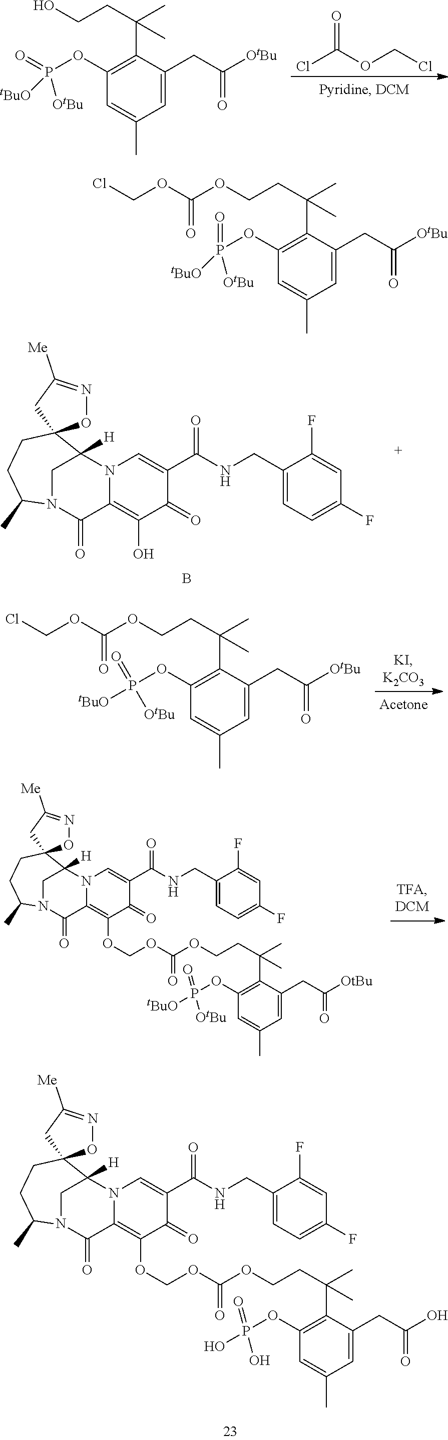

### Step 1: Preparation tert-butyl 2-(2-(4-(((chloromethoxy)carbonyl)oxy)-2-methylbutan-2-yl)-3-((di-tert-butoxyphosphoryl)oxy)-5-methylphenyl)acetate

The title compound was made following the same method as Example 19 step 1, except using tert-butyl 2-(3-((di-tert-butoxyphosphoryl)oxy)-2-(4-hydroxy-2-methylbutan-2-yl)-5-methylphenyl)acetate (prepared according to WO2023102239) instead of dibenzyl (4-(hydroxymethyl)phenyl) phosphate. MS (m/z) 593.9 [M+H]+.

### Step 2: Preparation tert-butyl 2-(3-((di-tert-butoxyphosphoryl)oxy-2-(4-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methylphenyl)acetate

The title compound was made following the same method as Example 19 step 2, except using tert-butyl 2-(2-(4-(((chloromethoxy)carbonyl)oxy)-2-methylbutan-2-yl)-3-((di-tert-butoxyphosphoryl)oxy)-5-methylphenyl)acetate instead of 4-((bis(benzyloxy)phosphoryl)oxy)benzyl (chloromethyl) carbonate. MS (m/z) 1043.9 [M+H]+.

### Step 3: Preparation 2-(2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yloxy)methoxy)carbonyloxy)-2-methylbutan-2-yl)-5-methyl-3-(phosphonooxy)phenyl)acetic acid (23)

To a mixture of tert-butyl 2-(3-((di-tert-butoxyphosphoryl)oxy)-2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methylphenyl)acetate (0.78 g, 0.75 mmol) in DCM (6 mL) at 0° C. was added TFA (0.8 mL). The resulting mixture was stirred at room temperature for 2 h. Solvent was removed under vacuum and the residue was purified by reverse phase preparative HPLC to afford the title compound. MS (m/z) 874.9 [M+H]+. 1H NMR (400 MHz, DMSO-d6) δ 10.27 (t, J=6.0 Hz, 1H), 8.69 (s, 1H), 7.47-7.36 (m, 1H), 7.30-7.15 (m, 2H), 7.12-7.02 (m, 1H), 6.69 (d, J=2.0 Hz, 1H), 5.77 (d, J=6.5 Hz, 1H), 5.58 (d, J=6.5 Hz, 1H), 4.62-4.55 (t, J=7.1 Hz, 4H), 3.88-3.77 (m, 4H), 3.66 (d, J=2.1 Hz, 2H), 2.99 (d, J=17.5 Hz, 1H), 2.61 (d, J=17.5 Hz, 1H), 2.18 (m, 5H), 1.95 (s, 3H), 1.75 (q, J=16.9, 13.2 Hz, 3H), 1.44 (d, J=2.0 Hz, 6H), 1.30-1.16 (m, 1H), 1.13 (m, 3H).

### Example 24: Preparation (2-(2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methyl-3-(phosphonooxy)phenyl)acetyl)-L-aspartic acid

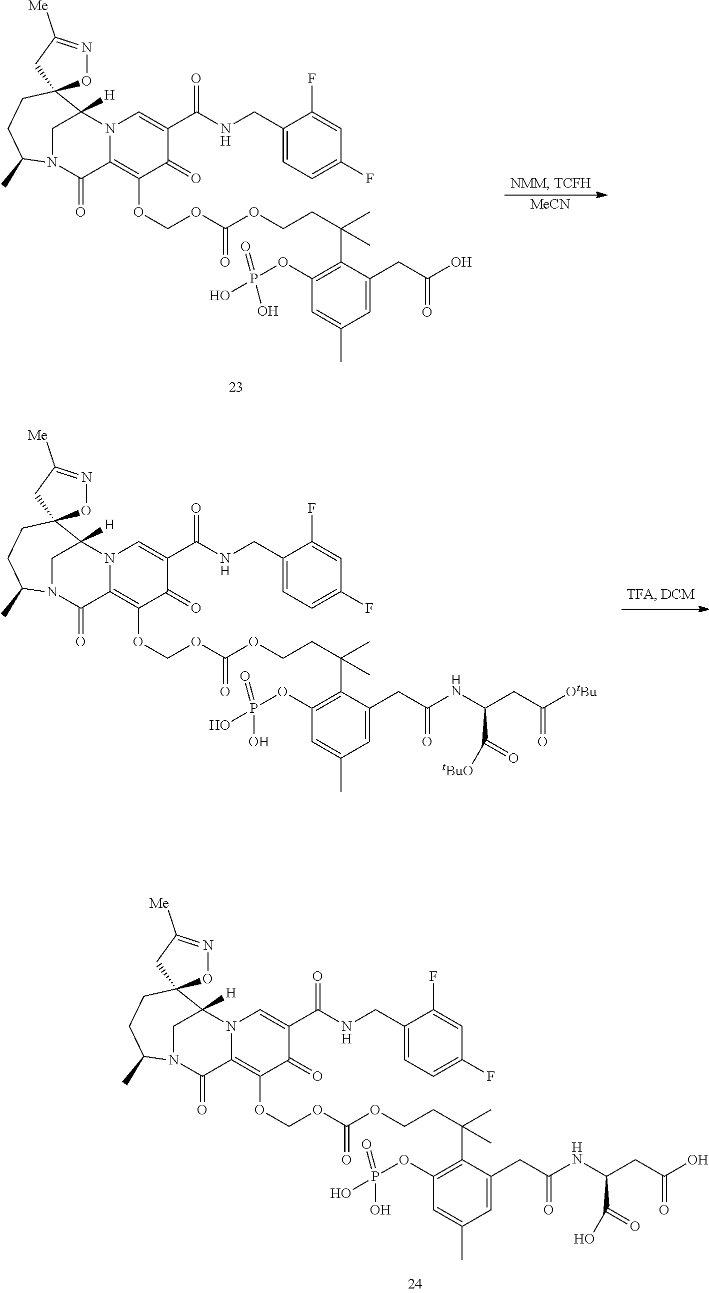

### Step 1: Preparation di-tert-butyl (2-(2-(4-((((3′S 5S,7′R)-10′-(2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methyl-3-(phosphonooxy)phenyl)acetyl)-L-aspartate

To a mixture of 2-(2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methyl-3-(phosphonooxy)phenyl)acetic acid (Example 23, 0.1 g, 0.114 mmol) and H-Asp(OtBu)-OtBu HCl (0.048 g, 0.17 mmol) in ACN (1 mL) at 0° C. was added N-methylmorpholine (0.017 g, 0.17 mmol) followed by TCFH (0.038 g, 0.137 mmol). The mixture was stirred at 0° C. for 30 minutes and then stirred at room temperature for 30 minutes. Then it was concentrated and the resulting residue was diluted with EtOAc, and treated with 0.1N HCl (until pH ˜2). The aqueous layer was extracted with EtOAc (3×) and the combined organic layer was washed with a 1:1 solution of brine/water and brine. The organic phase was dried over Na₂SO₄, filtered, concentrated and used directly in next step. MS (m/z) 1102.9 [M+H]+.

### Step 2: Preparation (2-(2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyloxy)-2-methylbutan-2-yl)-5-methyl-3-(phosphonooxy)phenyl)acetyl)-L-aspartic acid (24)

The title compound was made following the same method as Example 23 step 3, except using di-tert-butyl (2-(2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methyl-3-(phosphonooxy)phenyl)acetyl)-L-aspartate instead of tert-butyl 2-(3-((di-tert-butoxyphosphoryl)oxy)-2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methylphenyl)acetate. MS (m/z) 989.9 [M+H]+. 1H NMR (400 MHz, DMSO-d6) δ 10.27 (t, J=5.9 Hz, 1H), 8.68 (s, 1H), 8.18 (d, J=8.0 Hz, 1H), 7.42 (td, J=8.6, 6.5 Hz, 1H), 7.24 (ddd, J=10.5, 9.3, 2.6 Hz, 1H), 7.15 (d, J=1.8 Hz, 1H), 7.12-7.02 (m, 1H), 6.68 (d, J=2.0 Hz, 1H), 5.77 (d, J=6.4 Hz, 1H), 5.58 (d, J=6.4 Hz, 1H), 4.66-4.46 (m, 5H), 3.91-3.81 (m, 2H), 3.72 (s, 2H), 3.66 (d, J=2.3 Hz, 2H), 2.99 (d, J=17.6 Hz, 1H), 2.76-2.66 (m, 1H), 2.60 (dd, J=16.8, 7.0 Hz, 2H), 2.17 (s, 4H), 2.17 (d, J=14.9 Hz, 1H), 1.95 (s, 3H), 1.81-1.67 (m, 3H), 1.44 (d, J=4.2 Hz, 6H), 1.26 (t, J=12.9 Hz, 1H), 1.13 (d, J=6.7 Hz, 3H).

### Example 25: Preparation 2-(2-(4-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)-2-methylbutan-2-yl)-5-methyl-3-(phosphonooxy)phenyl)acetic acid (25)

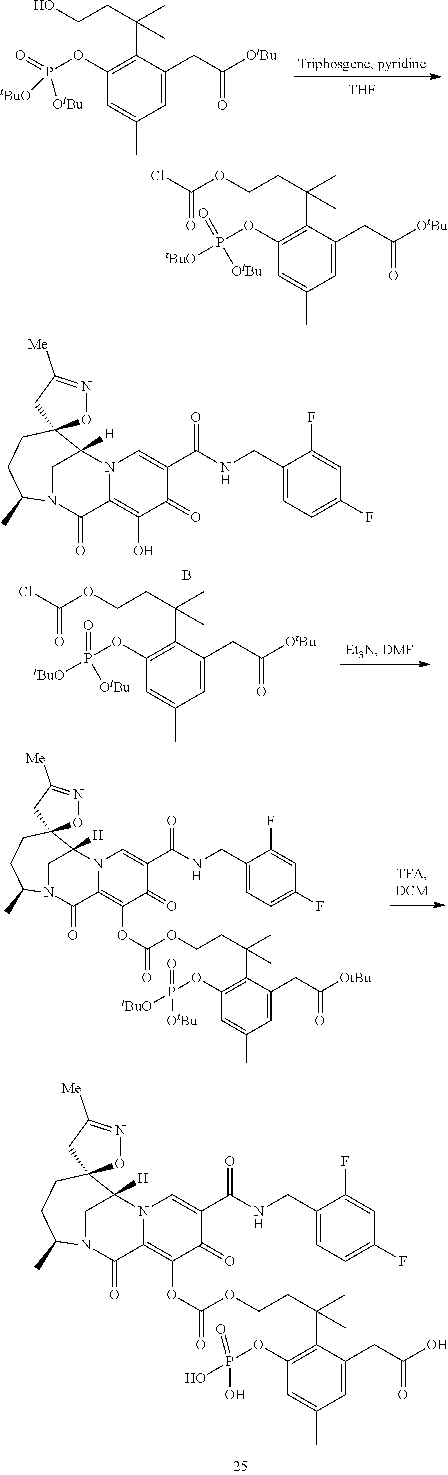

### Step 1: Preparation tert-butyl 2-(2-(4-((chlorocarbonyl)oxy)-2-methylbutan-2-yl)-3-((di-tert-butoxyphosphoryl)oxy)-5-methylphenyl)acetate

The title compound was made following the same method as Example 1 step 2, except using tert-butyl 2-(3-((di-tert-butoxyphosphoryl)oxy)-2-(4-hydroxy-2-methylbutan-2-yl)-5-methylphenyl)acetate (prepared according to WO2023102239) instead of di-tert-butyl ((2-(methylamino)pyridin-3-yl)methyl) phosphate. MS (m/z) 563.9 [M+H]+.

### Step 2. Preparation of tert-butyl 2-(3-((di-tert-butoxyphosphoryl oxy)-2-(4-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)-2-methylbutan-2-yl)-5-methylphenyl)acetate

The title compound was made following a similar procedure as Example 1 step 3, except using tert-butyl 2-(2-(4-((chlorocarbonyl)oxy)-2-methylbutan-2-yl)-3-((di-tert-butoxyphosphoryl)oxy)-5-methylphenyl)acetate instead of di-tert-butyl ((2-((chlorocarbonyl)(methyl)amino)pyridin-3-yl)methyl) phosphate. DMF was used as solvent instead of DCE and triethylamine was used instead of N,N-diisopropylethylamine and no TFA treatment. MS (m/z) 1013.96 [M+H]+.

### Step 3: Preparation 2-(2-(4-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)-2-methylbutan-2-yl)-5-methyl-3-(phosphonooxy)phenyl)acetic acid

The title compound was made following the same method as Example 23, step 3, except using tert-butyl 2-(3-((di-tert-butoxyphosphoryl)oxy)-2-(4-(((((3′S,5S,7′R)-1′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)-2-methylbutan-2-yl)-5-methylphenyl)acetate instead of tert-butyl 2-(3-((di-tert-butoxyphosphoryl)oxy)-2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methylphenyl)acetate. MS (m/z) 844.77 [M+H]+. 1H NMR (400 MHz, DMSO-d6) δ 10.11 (t, J=6.0 Hz, 1H), 8.83 (s, 1H), 7.41 (td, J=8.7, 6.6 Hz, 1H), 7.29-7.17 (m, 2H), 7.12-7.02 (m, 1H), 6.70 (d, J=2.0 Hz, 1H), 4.70 (d, J=2.4 Hz, 1H), 4.55 (tq, J=14.8, 7.6, 5.8 Hz, 3H), 3.95 (t, J=7.4 Hz, 2H), 3.78 (d, J=2.6 Hz, 2H), 3.70 (d, J=15.7 Hz, 1H), 3.01 (d, J=17.5 Hz, 1H), 2.64 (d, J=17.6 Hz, 1H), 2.32-2.17 (m, 6H), 2.09 (d, J=4.6 Hz, 1H), 1.94 (s, 3H), 1.82-1.72 (m, 3H), 1.46 (s, 6H), 1.27-1.13 (m, 3H).

### Example 26: Preparation (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 3-(2,4-dimethyl-6-(phosphonooxy)phenyl)-3-methylbutanoate (26)

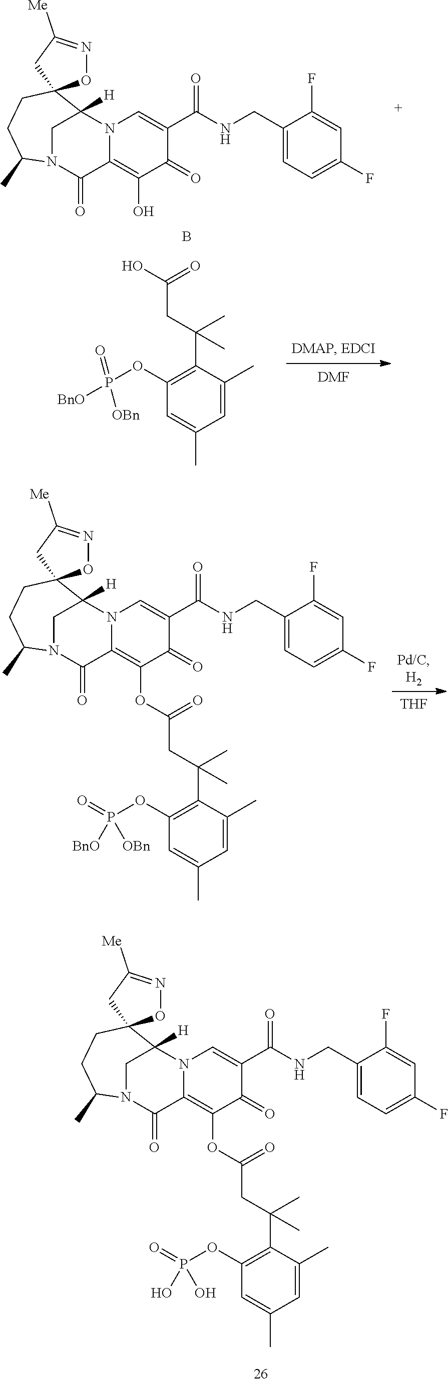

### Step 1: Preparation of (3′,5S,7′R)-10′-((2,4-difluorobenzyl)carbamoyl)-3,3′-dimethyl-1′,11′-dioxo-1′,4′,5′,11″-tetrahydro-3′H,4H,7′H-spiro[isoxazole-5,6′-[2,7]methanopyrido[1,2-a][1,4]diazonin]-12′-yl 3-(2-((bis(benzyloxy)phosphoryl)oxy)-4.64dimethylphenyl)-3-methylbutanoate

To mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (0.55 g, 1.13 mmol) in DMF was added 3-(2-dibenzyloxyphosphoryloxy-4,6-dimethyl-phenyl)-3-methyl-butanoic acid (prepared according to Chem. Sci. 2021, 12, 10076) (0.818 g, 1.7 mmol), 4-dimethylaminopyridine (0.173 g, 1.4 mmol) and followed by EDC HCl (0.32 g, 1.7 mmol). The mixture was stirred at rt for overnight. The mixture was diluted with EtOAc, and washed with LiCl (5%) and brine, and dried over MgSO₄. The solvent was removed under vacuo and the resulting residue was purified by silica gel column to afford the title compound. MS (m/z) 951.4[M+H]+.

### Step 2: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-vi 3-(2,4-dimethyl-6-(phosphonooxy)phenyl)-3-methylbutanoate (26)

The title compound was made following the same method as Example 19, step 3, except using (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 3-(2-((bis(benzyloxy)phosphoryl)oxy)-4,6-dimethylphenyl)-3-methylbutanoate instead of 4-((bis(benzyloxy)phosphoryl)oxy)benzyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate. MS (m/z) 770.6 [M+H]+. 1H NMR (400 MHz, DMSO-d6) δ 10.14 (t, J=6.0 Hz, 1H), 8.78 (s, 1H), 7.39 (td, J=8.7, 6.6 Hz, 1H), 7.24 (td, J=9.8, 2.6 Hz, 1H), 7.12-7.02 (m, 2H), 6.63 (d, J=2.0 Hz, 1H), 4.67 (s, 1H), 4.54 (qd, J=14.7, 5.6 Hz, 3H), 3.69 (d, J=14.2 Hz, 1H), 3.16 (s, 3H), 3.00 (d, J=17.4 Hz, 1H), 2.64 (d, J=17.3 Hz, 1H), 2.48 (s, 3H), 2.16 (s, 3H), 1.94 (s, 3H), 1.79 (s, 3H), 1.75 (q, J=6.9, 5.1 Hz, 1H), 1.63 (d, J=5.9 Hz, 6H), 1.16 (d, J=6.7 Hz, 3H).

### Example 27: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3-(2,4-dimethyl-64phosphonooxy)phenyl)-3-methylbutanoate (27)

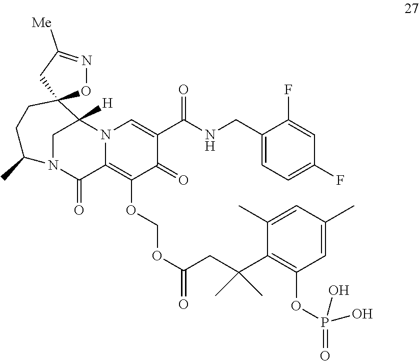 [SMILES: CC1=NO[C@@]2(CC[C@H](C)N3C[C@H]2n2cc(C(=O)NCc4ccc(F)cc4F)c(=O)c(OCOC(=O)CC(C)(C)c4c(C)cc(C)cc4OP(=O)(O)O)c2C3=O)C1]

The title compound was prepared following the same method as Example 20, except using 3-(2-dibenzyloxy phosphoryloxy-4,6-dimethyl-phenyl)-3-methyl-butanoic acid (prepared according to Chem. Sci. 2021, 12, 10076) instead of 3-(2-(2-(tert-butoxy)-2-oxoethyl)-6-((di-tert-butoxyphosphoryl)oxy)-4-methylphenyl)-3-methylbutanoic acid in step 1. MS (m/z) 800.85 [M+H]+. 1H NMR (400 MHz, DMSO-d6) δ 10.29 (t, J=5.9 Hz, 1H), 8.67 (s, 1H), 7.42 (td, J=8.6, 6.6 Hz, 1H), 7.25 (ddd, J=10.3, 9.3, 2.6 Hz, 1H), 7.12-7.01 (m, 2H), 6.62 (d, J=1.9 Hz, 1H), 5.68 (d, J=6.2 Hz, 1H), 5.47 (d, J=6.2 Hz, 1H), 4.70-4.56 (m, 2H), 4.59-4.47 (m, 2H), 3.68 (s, 2H), 2.99 (dd, J=17.2, 2.9 Hz, 2H), 2.87 (d, J=17.0 Hz, 1H), 2.61 (d, J=17.6 Hz, 1H), 2.42 (s, 3H), 2.14 (s, 3H), 1.95 (s, 3H), 1.82 (q, J=6.5, 5.5 Hz, 1H), 1.81-1.69 (m, 2H), 1.48 (d, J=4.9 Hz, 6H), 1.36-1.24 (m, 1H), 1.16 (d, J=6.7 Hz, 3H).

### Example 28: Preparation (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3-(2,4-dimethyl-6-(phosphonooxy)phenyl)-3-methylbutanoate (28)

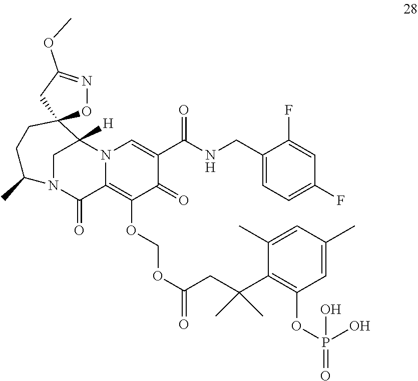 [SMILES: COC1=NO[C@@]2(CC[C@H](C)N3C[C@H]2n2cc(C(=O)NCc4ccc(F)cc4F)c(=O)c(OCOC(=O)CC(C)(C)c4c(C)cc(C)cc4OP(=O)(O)O)c2C3=O)C1]

The title compound was made in a similar manner as Example 27, except using (3′S,5S,7′R)—N-(2,4-difluorobenzyl)-12′-hydroxy-3-methoxy-3′-methyl-1′,11′-dioxo-1′,4′,5′,11″-tetrahydro-3′H,4H,7′H-spiro[isoxazole-5,6′-[2,7]methanopyrido[1,2-a][1,4]diazonine]-10′-carboxamide instead of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide. MS (m/z) 816.97 [M+H]+. 1H NMR (400 MHz, DMSO-d6) δ 10.29 (t, J=5.9 Hz, 1H), 8.71 (s, 1H), 7.42 (td, J=8.7, 6.6 Hz, 1H), 7.24 (ddd, J=10.5, 9.3, 2.6 Hz, 1H), 7.12-7.01 (m, 2H), 6.67-6.58 (m, 1H), 5.67 (d, J=6.2 Hz, 1H), 5.46 (d, J=6.2 Hz, 11H), 4.70 (d, J=2.1 Hz, 11H), 4.64 (ddd, J=16.9, 14.9, 8.2 Hz, 1H), 4.58-4.47 (m, 2H), 3.82 (s, 3H), 3.02 (t, J=16.6 Hz, 2H), 2.87 (d, J=16.9 Hz, 1H), 2.73 (d, J=16.9 Hz, 1H), 2.42 (s, 3H), 2.16 (d, J=22.0 Hz, 4H), 1.89 (dd, J=15.4, 6.3 Hz, 1H), 1.87-1.69 (m, 2H), 1.63 (d, J=6.3 Hz, 1H), 1.48 (d, J=4.8 Hz, 6H), 1.36-1.12 (m, 1H), 1.16 (d, J=6.7 Hz, 3H).

### Example 29: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-(4-(phosphonooxy)phenyl)acetate (29)

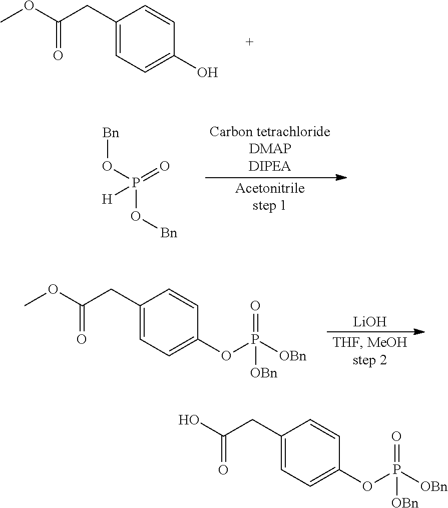

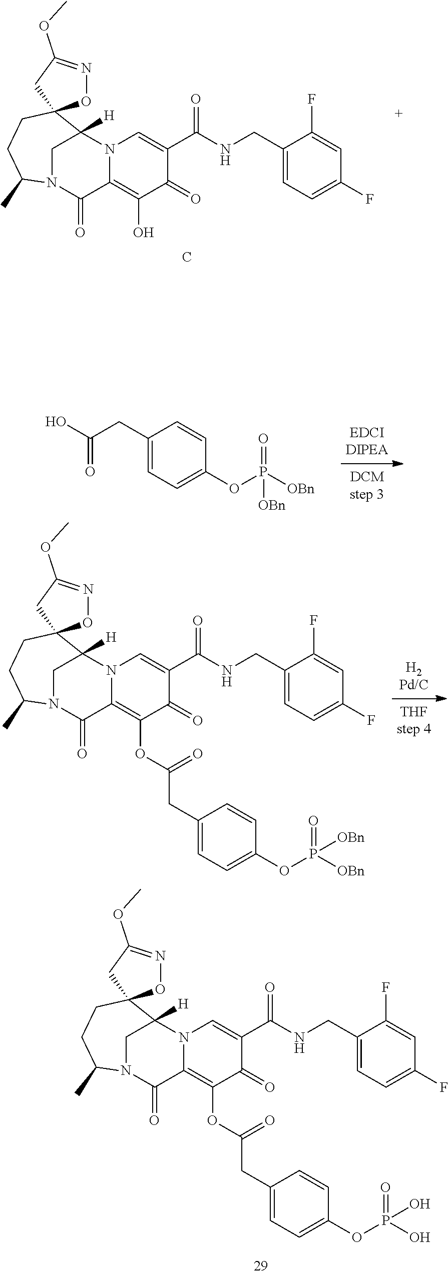

### Step 1: Synthesis of Methyl 2-(4-((bis(benzyloxy)phosphoryl)oxy)phenyl)acetate

In a 500 mL flask, to a solution of methyl 2-(4-hydroxyphenyl)acetate (1.89 g, 11.4 mmol) in acetonitrile (90 mL) at −10° C. was added CCl₄ (5.5 mL, 57 mmol), DIPEA (4.16 mL, 23.9 mmol) and DMAP (139 mg, 1.14 mmol). After stirring for 10 mins, to the mixture was added dibenzyl phosphite (3.7 mL, 16.5 mmol) slowly to keep the temperature below −10° C. The reaction was stirred at −10° C. for 1 h. the reaction was quenched with aqueous K₂HPO₄ (0.5 M) in acetonitrile (90 mL) and allowed to stir at room temperature for 15 min. The mixture was extracted with ethyl acetate (3×150 mL). The combined organic layers were washed with water and brine, dried over Na₂SO₄, filtered and concentrated under vacuum. The residue was purified on silica gel chromatography, eluting with 30-50% EtOAc/hexane to afford the title compound. MS calculated for C₂₃H₂₃O₆P 426.12, Found MS (ESI+): 426.23 [M]+.

### Step 2: Synthesis of 2-(4-((bis(benzyloxy)phosphoryl)oxy)phenyl)acetic acid

LiOH (1N, 9 mL, 5 mmol) was added into a mixture of methyl 2-(4-(bis(benzyloxy)phosphoryloxy)phenyl)acetate (1.53 g, 3.59 mmol) in THF (15 mL) and methanol (5 mL) at 0° C. The reaction was stirred at 0° C. for 3.5 h, then stirred for 0.5 h at room temperature. The mixture was cooled back to 0° C., and acidified by IN HCl to pH=2. The mixture was concentrated and extracted by ethyl acetate (3×50 mL). The extraction was dried over Na₂SO₄ and concentrated to give the title compound. MS calculated for C₂₂H₂₁O₆P: 412.11, Found MS (ESI+): 412.88 [M+H].

### Step 3: Synthesis of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-(4-((bis(benzyloxy)phosphoryl)oxy)phenyl)acetate

(3′S,5S,7′R)—N-(2,4-Difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate C, 450 mg, 0.896 mmol) and 2-(4-((bis(benzyloxy)phosphoryl)oxy)phenyl)acetic acid (823 mg, 2 mmol) were dissolved in DCM (10 mL) at room temperature. DIPEA (347 mg, 2.69 mmol) and DMAP (87.5 mg, 0.7 mmol) were added sequentially, then EDCI (400 mg, 0.21 mmol) was added. The reaction mixture was stirred at rt for 17 hr and the reaction mixture was concentrated. The residue was partitioned between EtOAc (50 mL) and aqueous NH₄Cl solution (saturated) (50 mL). The organic phase was separated and washed with water (50 mL) and brine (50 mL) and then was concentrated to dryness. The residue was purified on silica gel column with 0-100% EtOAc/hexane to afford title compound. MS calculated for C₄₆H₄₃F₂N₄O₁₁P: 896.26, Found MS (ESI+): 896.77 [M+H].

### Step 4: Synthesis of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) 2-(4-(phosphonooxy)phenyl)acetate

(3′S,5S,7′R)-10′-((2,4-Difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-(4-((bis(benzyloxy)phosphoryl)oxy)phenyl)acetate (189 mg, 0.211 mmol) was dissolved in THF (21 mL) at rt. Pd/C (5%) (44.5 mg, 0.0211 mmol) was added with stirring. Hydrogenolysis was undertaken with supply of hydrogen (g) balloon for 2 hrs. The reaction mixture was then filtered to remove Pd catalyst. The filtrate was concentrated to dryness to afford the title compound (29). MS calculated for C₃₂H₃₁F₂N₄O₁₁P: 716.17, Found MS (ESI+): 717.10 [M+H]. ¹H NMR (400 MHz, CD3CN) δ 10.19 (s, 1H), 8.56 (s, 1H), 7.48-7.38 (m, 1H), 7.35 (d, J=8.3 Hz, 2H), 7.18 (d, J=8.2 Hz, 2H), 7.02-6.92 (m, 2H), 4.81-4.63 (m, 1H), 4.59 (d, J=6.0 Hz, 2H), 4.40 (s, 1H), 3.91 (d, J=4.9 Hz, 2H), 3.88 (s, 3H), 3.74 (s, 2H), 3.01 (d, J=17.2 Hz, 1H), 2.71 (d, J=17.3 Hz, 1H), 1.95-1.78 (m, 3H), 1.54-1.43 (m, 1H), 1.21 (d, J=6.7 Hz, 3H).

### Example 30: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 2-(4-(phosphonooxy)phenyl)acetate (30)

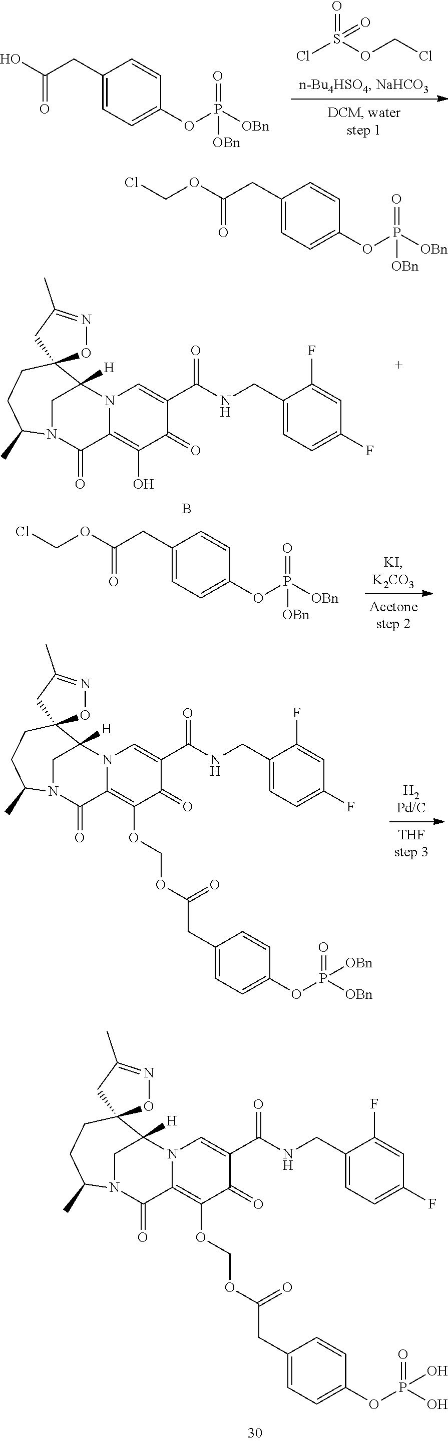

### Step 1: Synthesis of Chloromethyl 2-(4-((bis(benzyloxy)phosphoryl)oxy)phenyl)acetate

2-(4-((bis(benzyloxy)phosphoryl)oxy)phenyl)acetic acid (710 mg, 1.72 mmol) was mixed with DCM (10 mL) and water (5 mL). NaHCO₃ (525 mg, 8.61 mmol) and tetrabutylammonium hydrogen sulfate (58.5 mg, 0.172 mmol) were added sequentially. Then chloromethyl sulfurochloridate (639 mg, 3.87 mmol) was added last. The resulting reaction mixture was stirred vigorously overnight. EtOAc (10 mL) and water (10 mL) were added and the organic phase was separated and concentrated to dryness. The residue was purified on silica gel column eluted with 0-100% EtOAc/Hexane to afford title compound. MS calculated for C₂₃H₂₂ClO₆P: 460.08, Found MS (ESI+): 460.89 [M+H].

### Step 2: Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,41H,7′H-spiro[isoxazole-5,6′-[2,7]methanopyrido[1,2-a][1,4]diazonin]-12′-yl)oxy)methyl 2-(4-((bis(benzyloxy)phosphoryl)oxy)phenyl)acetate

(3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 110 mg, 0.226 mmol), K₂CO₃ (62.5 mg, 0.452 mmol) and KI (48.8 mg, 0.294 mmol) were mixed with acetone (10 mL) at room temperature. Then a solution of chloromethyl 2-(4-((bis(benzyloxy)phosphoryl)oxy)phenyl)acetate (125 mg, 0.271 mmol) in acetone (0.2 mL) was added. The reaction mixture was stirred at rt for 17 hr. The reaction mixture was partitioned between EtOAc (10 mL) and aqueous saturated NH₄Cl (10 mL). The organic layer was separated and concentrated to dryness. The residue was purified with silica gel column chromatography, eluting with 0-100% EtOAc/hexane and then reverse phase preparative HPLC, eluting with acetonitrile and water (neural mobile phase) to afford title compound. MS calculated for C₄₇H₄₅F₂N₄O₁₁P: 910.28, Found MS (ESI+): 911.01 [M+H].

### Step 3: Preparation of ((3′S,5S,7′R)-10′-((2,4-difluorobenzyl carbamoyl)-3,3′-dimethyl-1′11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 2-(4-(phosphonooxy)phenyl)acetate (30)

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 2-(4-((bis(benzyloxy)phosphoryl)oxy)phenyl)acetate (10 mg, 0.011 mmol) was dissolved in THF (5 mL). Pd/C (5%) (2.34 mg, 0.0011 mmol) was added. The hydrogenolysis was undertaken with H₂ gas balloon for 2 hrs. The reaction mixture was filtered to remove Pd catalyst. The filtrate was concentrated to afford the title compound (30). MS calculated for C33H33F2N40P: 730.19, Found MS (ESI+): 730.80 [M+H]. ¹H NMR (400 MHz, CD₃CN) δ 10.21 (s, 1H), 8.60 (s, 1H), 7.50-7.35 (m, 1H), 7.30 (d, J=8.3 Hz, 2H), 7.20 (d, J=8.2 Hz, 2H), 7.00-6.90 (m, 2H), 5.88 (d, J=6.5 Hz, 1H), 5.83 (d, J=6.6 Hz, 1H), 4.80-4.60 (m, 1H), 4.59 (d, J=6.0 Hz, 2H), 4.38 (s, 1H), 3.89 (d, J=4.9 Hz, 2H), 3.71 (s, 2H), 3.05 (d, J=17.2 Hz, 1H), 2.73 (d, J=17.3 Hz, 1H), 1.99 (s, 3H), 1.95-1.80 (m, 3H), 1.55-1.41 (m, 1H), 1.22 (d, J=6.7 Hz, 3H).

### Example 31: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-(phosphonooxy)benzyl) carbonate (31)

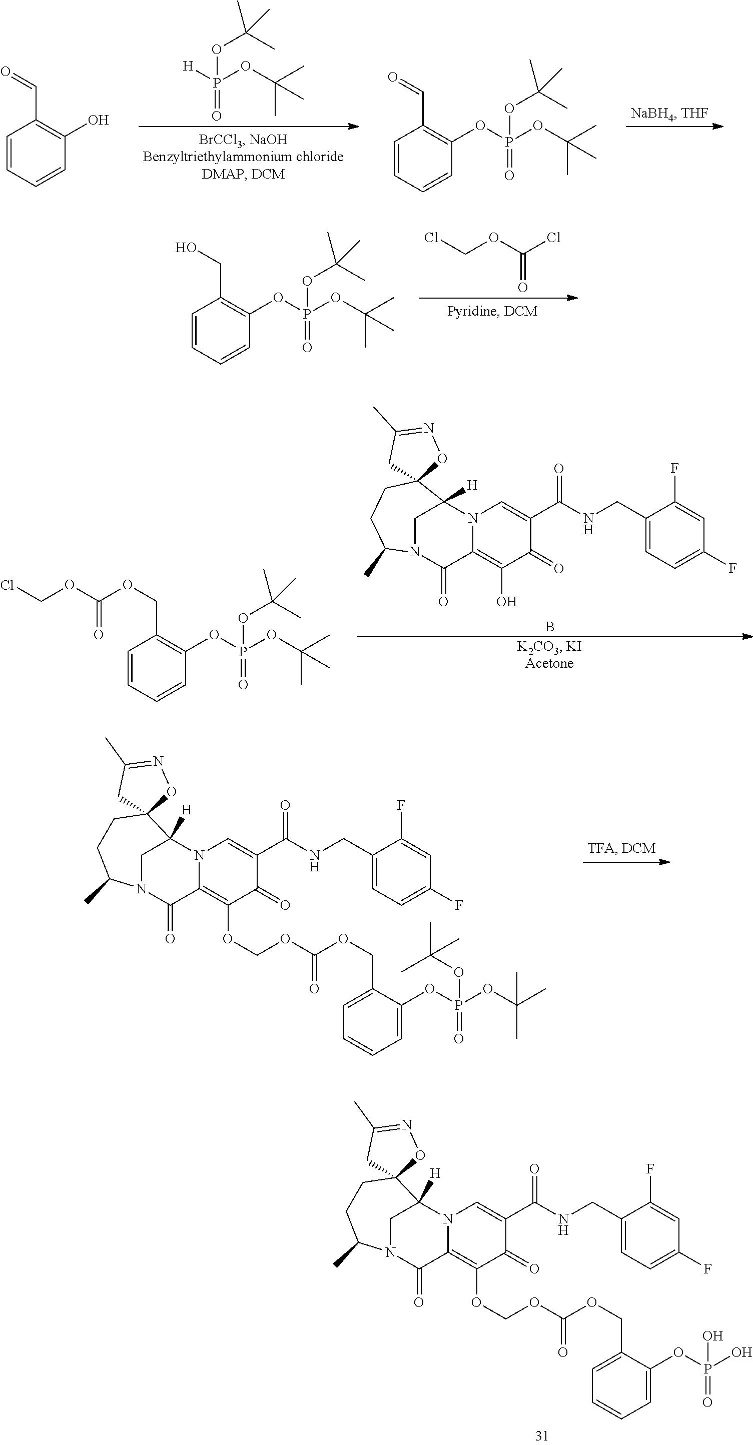

### Step 1: Synthesis of Di-tert-butyl (2-formylphenyl) phosphate

Benzyltriethylammonium chloride (400 mg, 1.76 mmol), DCM (30 mL), and bromotrichloromethane (3.25 g, 16.4 mmol), were added to a solution of sodium hydroxide (4.7 g) in water (30 mL). To this biphasic mixture, vigorously stirred at 0° C., was added a solution of 2-tert-butoxyphosphonoyloxy-2-methyl-propane (2.956 g, 15.2 mmol) in DCM (30 mL) dropwise over 5 min. The reaction mixture was then allowed to warm to room temperature while stirring vigorously for 2 h. at which point, a solution of 2-hydroxybenzaldehyde (1.43 g, 11.7 mmol) in DCM (30 mL) and DMAP (138 mg, 1.17 mmol) were added. The mixture was stirred for 1 h. The organic layer was then separated and washed with 10% aqueous citric acid solution and dried over anhydrous MgSO₄. After removing the solvent, the residue was purified by column chromatography on silica gel to provide title product. MS (m/z) 336.79 [M+Na]⁺.

### Step 2: Synthesis of Di-tert-butyl (2-(hydroxymethyl)phenyl) phosphate

Di-tert-butyl (2-formylphenyl) phosphate (3.5 g, 11.1 mmol) was dissolved in THF (25 mL) and cooled to 0° C. followed by addition of a solution of NaBH₄ (421 mg, 11.1 mmol) in water (5 mL). The resulting reaction mixture was stirred for 30 min then diluted with water (25 ml). The reaction mixture was extracted with EtOAc, washed with saturated aqueous NaHCO₃, and dried over anhydrous MgSO₄. After removal of the solvent, the residue was purified by column chromatography on silica gel to provide title product. MS (m/z) 338.875 [M+Na]⁺.

### Step 3: Synthesis of Chloromethyl (2-((di-tert-butoxyphosphoryl)oxy)benzyl) carbonate

The title compound was synthesized by the same procedure as Example 19, step 1, except starting from di-tert-butyl (2-(hydroxymethyl)phenyl) phosphate (1.99 g, 6.29 mmol) instead of dibenzyl (4-(hydroxymethyl)phenyl) phosphate. MS (m/z) 430.791 [M+Na].

### Step 4: Synthesis of 2-di-tert-butoxyphosphoryl)oxy)benzyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

The title compound was synthesized by the same procedure as Example 19, step 2, except starting from chloromethyl (2-((di-tert-butoxyphosphoryl)oxy)benzyl) carbonate (504 mg, 1.23 mmol) instead of chloromethyl (4-dibenzyloxyphosphoryloxyphenyl)methyl carbonate. MS (m/z) 859.74 [M+H]⁺.

### Step 5: Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-(phosphonooxy)benzyl) carbonate (31)

The title compound was synthesized by the same procedure as Example 20, step 3, except starting from 2-((di-tert-butoxyphosphoryl)oxy)benzyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (55 mg, 0.064 mmol) instead of (((3′S,5S,7′R)-10′-((2,4-difluorobenzyl)carbamoyl)-3,3′-dimethyl-1′,11′-dioxo-1′,4′,5′,11′-tetrahydro-3′H,4H,7H-spiro[isoxazole-5,6′-[2,7]methanopyrido[1,2-a][1,4]diazonin]-12′-yl)oxy)methyl 3-(2-(2-(tert-butoxy)-2-oxoethyl)-6-((di-tert-butoxyphosphoryl)oxy)-4-methylphenyl)-3-methylbutanoate. MS (m/z) 747.84 [M+H]⁺. ¹H NMR (400 MHz, DMSO-d6) δ 10.28 (s, 1H), 8.70 (s, 1H), 7.48-7.24 (m, 5H), 7.17-7.00 (m, 2H), 5.87 (d, J=6.5 Hz, 1H), 5.68 (d, J=6.6 Hz, 1H), 5.20 (s, 2H), 4.59 (d, J=26.2 Hz, 4H), 3.75-3.63 (m, 2H), 3.00 (s, 1H), 2.63 (s, 1H), 1.94 (s, 3H), 1.74 (d, J=19.7 Hz, 3H), 1.26 (s, 1H), 1.11 (d, J=6.6 Hz, 3H).

### Example 32: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2-(phosphonooxy)benzyl) carbonate (32)

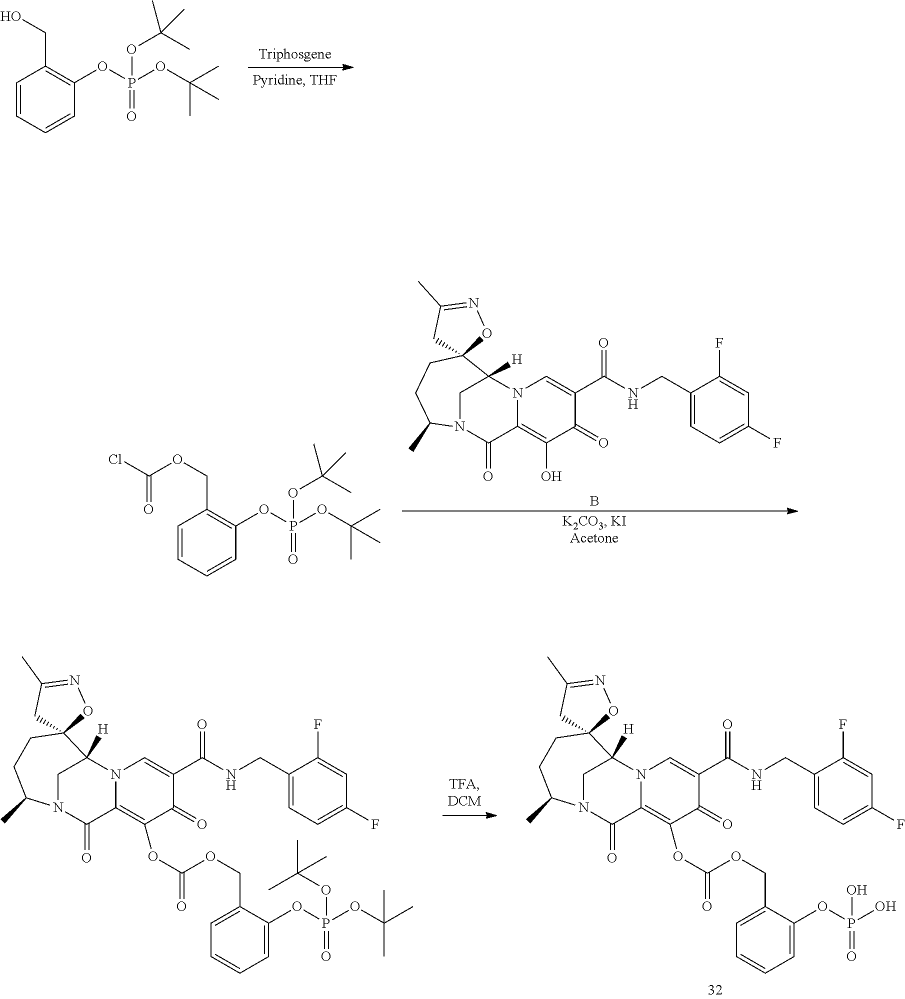

### Step 1: Synthesis of 2-((di-tert-butoxyphosphoryl)oxy)benzyl carbonochloridate

The title compound was synthesized following the same procedure as Example 25, step 1, except using di-tert-butyl (2-(hydroxymethyl)phenyl) phosphate (prepared in Example 31) (550 mg, 1.74 mmol) instead of tert-butyl 2-(3-((di-tert-butoxyphosphoryl)oxy)-2-(4-hydroxy-2-methylbutan-2-yl)-5-methylphenyl)acetate.

### Step 2: Synthesis of 2-((di-tert-butoxyphosphoryl)oxy)benzyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate

The title compound was synthesized followed the same procedure as Example 25, step 2, except using 2-((di-tert-butoxyphosphoryl)oxy)benzyl carbonochloridate instead of tert-butyl 2-(2-(4-((chlorocarbonyl)oxy)-2-methylbutan-2-yl)-3-((di-tert-butoxyphosphoryl)oxy)-5-methylphenyl)acetate. MS (m/z) 829.739 [M+H]1.

### Step 3: Synthesis of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2-(phosphonooxy)benzyl) carbonate (32)

The title compound was synthesized following the same procedure as Example 20, step 3, except starting from 2-((di-tert-butoxyphosphoryl)oxy)benzyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate (100 mg, 0.121 mmol) instead of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3-(2-(2-(tert-butoxy)-2-oxoethyl)-6-((di-tert-butoxyphosphoryl)oxy)-4-methylphenyl)-3-methylbutanoate. MS (m/z) 717.753 [M+H]¹. ¹H NMR (400 MHz, DMSO-d6) δ 10.12 (t, J=6.0 Hz, 1H), 8.86 (s, 1H), 7.53-7.28 (m, 4H), 7.29-7.16 (m, 2H), 7.14-7.04 (m, 1H), 5.33 (s, 2H), 4.72 (s, 1H), 4.66-4.46 (m, 3H), 3.83 (dd, J=15.3, 2.6 Hz, 1H), 3.72 (dd, J=15.1, 1.9 Hz, 1H), 3.03 (d, J=17.4 Hz, 1H), 2.68 (s, 1H), 1.95 (s, 3H), 1.87-1.73 (m, 3H), 1.31-1.22 (m, 1H), 1.17 (d, J=6.7 Hz, 3H).

### Example 33: Preparation of benzyl N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)-N-(((phosphonooxy)methoxy)carbonyl)glycinate (33)

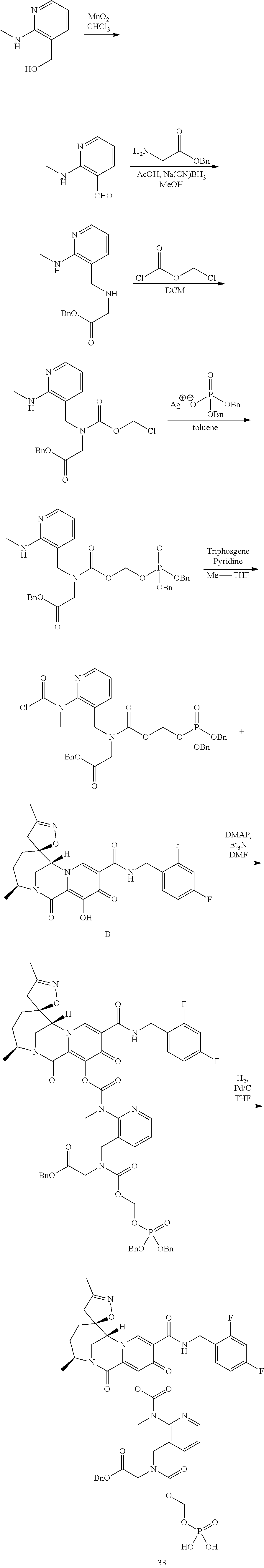

### Step 1: Preparation of 2-(methyl)amino)nicotinaldehyde

In a 1 liter reaction flask, (2-(methylamino)pyridin-3-yl)methanol (50.0 g, 632 mmol) was mixed with CHCl₃ (500 mL) at rt. MnO₂ (157 g, 1810 mmol) was added under N₂. Reaction mixture was warmed to 60-65 TC and stirred at 60-65 TC for 16 hrs. Reaction mixture was cooled to rt. The resulting reaction mixture was filtered. Filtrate was treated with a solution of Na₂SO₃ (40.0 g) in 400 mL water. The solid on filter was washed with DCM (1 liter). The aqueous layer was extracted with DCM (300 mL×2). The combined organic layers were dried over Na₂SO₄ and was filtered. The filtrate was concentrated to dryness to afford the product, 2-(methylamino)nicotinaldehyde, without the need of further purification. MS (m/z): calculated for C₇H₈N₂O: 136.06; found (+ESI): 136.87 [M+H]+.

### Step 2: Preparation of benzyl ((2-(methylamino)pyridin-3-yl)methyl)glycinate

In a 1 liter reaction flask, 2-(methylamino)nicotinaldehyde (40 g, 293 mmol) and benzyl glycinate (102 g, 771 mmol) were mixed with AcOH (17.6 g, 293 mmol) and MeOH (400 mL) at rt. Reaction mixture was warmed to 35-40° C. with stirring. In 30 min, the reaction mixture was cooled down to 0° C. NaBH₃CN (36.9 g, 587 mmol) was added at that temperature. Reaction mixture was then warmed up to rt and was stirred at that temperature for 2 hours. Reaction mixture was then concentrated to dryness. The residue was then portioned between DCM (300 ml) and the sat. NaHCO₃ (600 mL). Organic phase was separated. The aqueous phase was extracted with DCM (2×300 mL). The combined organic layers was dried over Na₂SO₄ and was filtered. The filtrate was concentrated to afford the crude product. Further purification on silica gel column with 0-50% EtOAc in Hexane afford product benzyl ((2-(methylamino)pyridin-3-yl)methyl)glycinate. MS (m/z): calculated for C₁₆H₁₉N₃O₂: 285.15; found (+ESI): 285.90 [M+H]+.

### Step 3: Preparation of benzyl N-((chloromethoxy)carbonyl)-N-((2-(methylamino)pyridin-3-yl)methyl)glycinate

In a 500 mL reaction flask benzyl ((2-(methylamino)pyridin-3-yl)methyl)glycinate (30 g, 89.3 mmol) was dissolved in DCM (315 mL) at rt. The solution was cooled to −5-0° C. Chloromethyl carbonochloridate (10.7 g, 83.1 mmol) was added dropwise at the same temperature. The reaction mixture was then stirred at the same temperature for 30 min. Sat. NaHCO₃ (400 mL) was then added at 0° C. The resulting reaction mixture was stirred at 0° C. for 10 mins. DCM (3×300 mL) was used for extraction. The combined organic layers were washed with sat. NaHCO₃ (3×200 mL) and was then dried over Na₂SO₄. The filtrate was concentrated to afford product benzyl N-((chloromethoxy)carbonyl)-N-((2-(methylamino)pyridin-3-yl)methyl)glycinate, which can be used without purification. MS (m/z): calculated for C18H20C1N3O4: 377.11; found (+ESI): 377.89 [M+H]+.

### Step 4: Preparation of benzyl N-((((bis(benzyloxy)phosphoryl)oxy)methoxy)carbonyl)-N-((2-(methylamino)pyridin-3-yl)methyl)glycinate

In a 1 liter reaction flask, benzyl N-((chloromethoxy)carbonyl)-N-((2-(methylamino)pyridin-3-yl)methyl)glycinate (36.0 g, 95.2 mmol) and silver(I) dibenzyl phosphate (110 g, 285 mmol) were mixed with toluene (26W mL) at rt. The resulting reaction mixture was heated up to 75-80° C. with stirring for 1 hr. The reaction mixture was then diluted with EtOAc (200 mL) and was then treated with brine (80 mL). The aqueous layer was extracted with EtOAc (2×200 mL). The combined organic layers were washed with brine (80 mL) and was then dried over Na₂SO₄. Filtrate after filtration was then concentrated to dryness. The residue was purified on silica gel column with 0-50% EtOAc/Hexane to afford product, benzyl N-((((bis(benzyloxy)phosphoryl)oxy)methoxy)carbonyl)-N-((2-(methylamino)pyridin-3-yl)methyl)glycinate. MS (m/z): calculated for C32H34N3O8P: 619.21; found (+ESI): 620.01 [M+H]. 1H NMR: (DMSO-d6 400 MHz): δ 7.97-7.95 (m, 1H), 7.37-7.23 (m, 16H), 6.46-6.42 (m, 1H), 6.01-5.90 (m, 1H), 5.61 (t, J=4 Hz, 2H), 5.13-4.97 (m, 6H), 4.35-4.33 (m, 2H), 4.12-4.05 (m, 2H), 2.82-2.78 (m, 3H).

### Step 5: Preparation of benzyl N-((((bis(benzyloxy)phosphoryl)oxy)methoxycarbonyl)-N-((2-((chlorocarbonyl)(methyl)amino)pyridin-3-yl)methyl)glycinate

In a 500 mL flask, benzyl N-((((bis(benzyloxy)phosphoryl)oxy)methoxy)carbonyl)-N-((2-(methylamino)pyridin-3-yl)methyl)glycinate (8.55 g, 13.8 mmol) was dissolved in Me-THF (100 mL) at rt. Under argon balloon, the solution was cooled down to 0° C. Then pyridine (1.417 g, 17.9 mmol) was added. 10 min later, triphosgene (3.76 g, 12.4 mmol) was added in one portion as solid. The cold bath was removed and the reaction mixture was stirred overnight. The reaction mixture was filtered and the solid was washed with Me-THF (100 mL) and EtOAc (100 mL). The filtrate was treated with HCl (IN) (200 mL) and brine (200 mL). The organic phase was then dried with Na₂SO₄ and was then concentrated to afford the crude product, benzyl N-((((bis(benzyloxy)phosphoryl)oxy)methoxy)carbonyl)-N-((2-((chlorocarbonyl)(methyl)amino)pyridin-3-yl)methyl)glycinate. MS (m/z): calculated for C33H33C1N₃O₉P: 681.16; found (+ESI): 681.86 [M+H]+.

### Step 6: Preparation of benzyl N-((((bis(benzyloxy)phosphoryl)oxy)methoxy)carbonyl)-N-((2-(((((3′S,5S,7′R)-10′-((2,4-<difluorobenzyl)carbamoyl)-3,3′-dimethyl-1′,11′-dioxo-1′,4′,5′,11′-tetrahydro-3′H,4H,7′H-spiro[isoxazole-5,6′-(2.71methanopyrido[1,2-a][1,4]diazonin]-12′-yl)oxy)carbonyl)(methyl)amino)pyridin-3-yl)methyl)glycinate

(3′S,5S,7′R)—N-(2,4-Difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 4.2 g, 8.63 mmol) and benzyl N-((((bis(benzyloxy)phosphoryl)oxy)methoxy)carbonyl)-N-((2-((chlorocarbonyl)(methyl)amino)pyridin-3-yl)methyl)glycinate (9.42 g, 13.8 mmol) were mixed in DMF (60 mL) at rt. Et₃N (2.62 g, 25.9 mmol) and DMAP (0.63 g, 5.18 mmol) were added sequentially. The reaction mixture was then stirred at rt for 2 hrs. DMF (40 mL) was added. The reaction mixture was stirred at rt for 17 hrs. The reaction mixture was then diluted with EtOAc (200 mL) and was washed with a mixture of sat NH₄Cl (100 mL), water (100 mL) and IN HCl (50 mL). The organic phase was separated and was treated with water (100 mL) and brine (100 mL) sequentially. After the removal of solvent, the crude product was purified by silica gel column chromatography, eluting with 0-100% EtOAc/hexane, to afford benzyl N-((((bis(benzyloxy)phosphoryl)oxy)methoxy)carbonyl)-N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)glycinate. MS (m/z): calculated for C₅₇H₅₆F₂N7O14P: 1131.36; found (+ESI): 1131.69 [M+H]+.

### Step 7: Preparation of benzyl N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)-N-(((phosphonooxy)methoxy)carbonyl)glycinate (33)

Benzyl N-((((bis(benzyloxy)phosphoryl)oxy)methoxy)carbonyl)-N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)glycinate (6.3 g, 5.57 mmol) was dissolved in THF (500 mL). Pd/C (5% loading) (1.23 g, 0.578 mmol) was added. The reaction mixture was purged with H₂ (gas) via vacuum-fill cycles 5 times, followed by bubbling with H₂ for 30 min. The reaction mixture was stirred placed under H₂ atmosphere for 4 hr. The reaction mixture was then filtered to remove Pd catalyst. The filtrate was concentrated to dryness. The residue was purified with reversed phase prep-HPLC on C18 column with water (containing 0.1% TFA) and acetonitrile (containing 0.1% TFA) to afford the product benzyl N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)-N-(((phosphonooxy)methoxy)carbonyl)glycinate after lyophilization MS (m/z): calculated for C₄₃H₄₄F₂N₇O₁₄P: 951.27; found (+ESI): 952.27 [M+H]+. ¹H NMR (400 MHz, DMSO-d₆) δ 10.17 (d, J=41.4 Hz, 1H), 8.78 (d, J=26.8 Hz, 1H), 8.43 (s, 1H), 7.99-7.70 (m, 1H), 7.50-7.15 (m, 8H), 7.05 (d, J=9.7 Hz, 1H), 5.62-5.26 (m, br, 2H), 5.25-4.97 (m, br, 2H), 4.96-4.38 (m, br, 6H), 4.37-3.99 (m, br, 2H), 3.95-3.53 (m, 2H), 3.51-3.09 (m, br, 3H), 3.09-2.77 (m, br, 1H), 2.73-2.56 (m, br, 1H), 1.93 (s, 3H), 1.86-1.57 (m, br, 3H), 1.49-0.96 (m, br, 4H).

### Example 34: Preparation of methyl N-((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)-N-(((phosphonooxy)methoxy)carbonyl)glycinate (34)

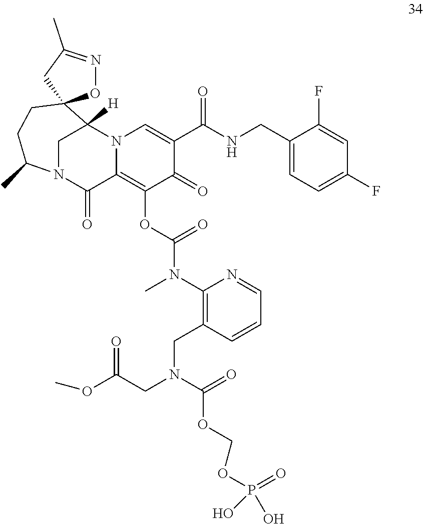 [SMILES: COC(=O)CN(Cc1cccnc1N(C)C(=O)Oc1c2n(cc(C(=O)NCc3ccc(F)cc3F)c1=O)[C@@H]1CN(C2=O)[C@@H](C)CC[C@]12CC(C)=NO2)C(=O)OCOP(=O)(O)O]

The title compound was prepared following the similar method for the preparation of Intermediate D steps 2 to 4 except using methyl glycinate hydrochloride in step 2 instead of tert-butyl glycinate and then following similar method for the preparation of Example 6 steps 1, 2 and 4. The resulting residue was purified by reverse phase preparative HPLC (5-100% MeCN. H₂O w/ 0.1% TFA). LCMS-ESI+ (m/z): calcd H+ for C₃₋₇H40F2N7O14P, Theoretical: 875.23, Found: 876.035. 1H NMR (400 MHz, Methanol-d4) δ 8.63 (d, J=28.6 Hz, 1H), 8.45 (s, 1H), 8.11-7.94 (m, 1H), 7.53-7.40 (m, 2H), 6.97 (q, J=9.6, 9.0 Hz, 2H), 5.71-5.43 (m, 2H), 5.13-4.04 (m, 11H), 3.90-3.60 (m, 4H), 3.56-3.47 (m, 1H), 3.40-3.34 (m, 1H), 3.21-3.05 (m, 1H), 2.69 (dd, J=18.0, 10.3 Hz, 1H), 2.11-2.01 (m, 3H), 1.92 (dd, J=24.6, 9.8 Hz, 3H), 1.24 (d, J=19.5 Hz, 3H).

### Example 35: Preparation of (phosphonooxy)methyl ((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)(2-hydroxyethyl)carbamate (35)

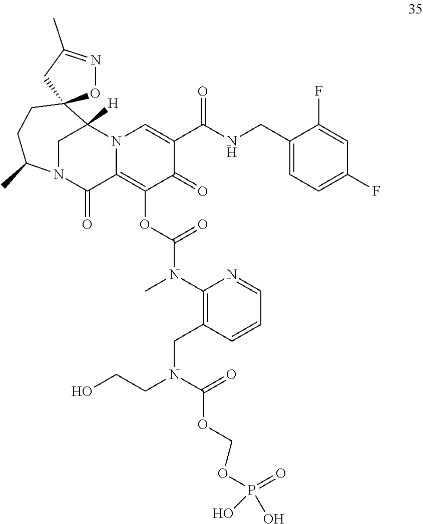 [SMILES: CC1=NO[C@@]2(CC[C@H](C)N3C[C@H]2n2cc(C(=O)NCc4ccc(F)cc4F)c(=O)c(OC(=O)N(C)c4ncccc4CN(CCO)C(=O)OCOP(=O)(O)O)c2C3=O)C1]

The title compound was prepared following the similar method for the preparation of Intermediate D steps 2 to 4 except using 2-((tert-butyldimethylsilyl)oxy)ethan-1-amine in step 2 instead of tert-butyl glycinate and then following similar method for the preparation of Example 6 steps 1, 2 and 4. The resulting residue was purified by reverse phase preparative HPLC (5-100% MeCN/H₂O w/ 0.1% TFA). LCMS-ESI+ (m/z): calcd H+ for C36H40F2N7O13P, Theoretical: 847.24. Found: 848.038. 1H NMR (400 MHz, Methanol-d4) δ 8.63 (d, J=30.9 Hz, 1H), 8.51-8.36 (m, 1H), 7.98-7.81 (m, 1H), 7.45 (d, J=6.9 Hz, 2H), 7.06-6.89 (m, 2H), 5.66 (dd, J=14.9, 7.9 Hz, 2H), 5.18-4.39 (m, 8H), 3.91-3.44 (m, 6H), 3.39 (d, J=6.8 Hz, 1H), 3.14 (dd, J=19.0, 10.2 Hz, 1H), 2.69 (dd, J=17.6, 7.9 Hz, 1H), 2.09-2.01 (m, 3H), 2.00-1.81 (m, 3H), 1.52-1.66 (m, 1H), 1.26 (td, J=15.6, 14.0, 7.1 Hz, 3H).

### Example 36: Preparation N-((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)-N-(((phosphonooxy)methoxy)carbonyl)glycyl-L-aspartic acid (36)

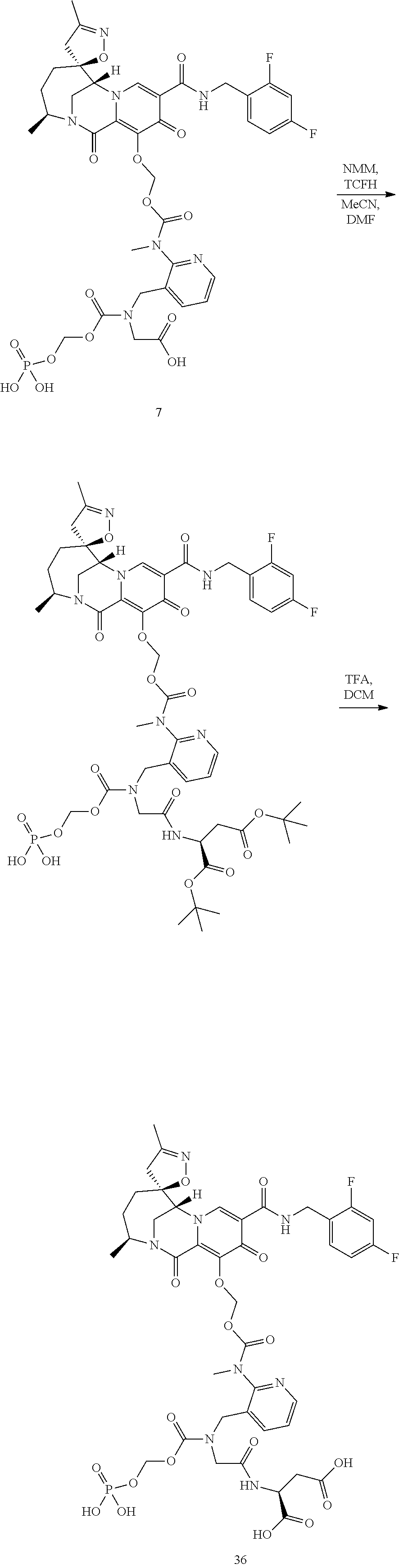

### Step 1: Preparation of di-tert-butyl N-((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)-N-(((phosphonooxy)methoxy)carbonyl)glycyl-L-aspartate

To a mixture of N-((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)-N-(((phosphonooxy)methoxy)carbonyl)glycine (Example 7, 0.053 g, 0.06 mmol) and H-Asp(OtBu)-OtBu HCl (0.025 g, 0.09 mmol) in ACN (1 mL) and DMF (0.5 mL) at 0° C. was added N-methylmorpholine (0.018 g, 0.17 mmol) followed by TCFH (0.02 g, 0.07 mmol). The mixture was stirred at 0° C. for 30 minutes and then stirred at room temperature for 30 minutes. Then it was concentrated and the resulting residue was purified by column chromatography to afford di-tert-butyl N-((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)-N-(((phosphonooxy)methoxy)carbonyl)glycyl-L-aspartate. MS (m/z) 1118.7 [M+H]+.

### Step 2: Preparation of N-((2-((((((3′S,5S,7′R)-10′-((2,4-difluorobenzyl)carbamoyl)-3,3′-dimethyl-1′,11′-dioxo-1′4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)-N-(((phosphonooxy)methoxy)carbonyl)glycyl-L-aspartic acid (36)

The title compound was made following the same method as Example 23 step 3, except using di-tert-butyl N-((2-((((((3′S,5S,7R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)-N-(((phosphonooxy)methoxy)carbonyl)glycyl-L-aspartate instead of tert-butyl 2-(3-((di-tert-butoxyphosphoryl)oxy)-2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methylphenyl)acetate. MS (m/z) 1006.60 [M+H]+. 1H NMR (400 MHz, Methanol-d4) δ 8.55 (s, 1H), 8.48-8.27 (m, 1H), 7.89 (d, J=8.6 Hz, 1H), 7.59-7.28 (m, 2H), 6.96 (s, 2H), 6.04-5.47 (m, 4H), 4.77-4.41 (m, 6H), 4.17-3.90 (m, 2H), 3.81 (s, 2H), 3.16 (d, J=21.8 Hz, 4H), 2.73 (d, J=32.2 Hz, 3H), 2.20-1.74 (m, 7H), 1.55 (dd, J=50.6, 36.4 Hz, 1H), 1.29 (d, J=6.7 Hz, 3H).

### Example 37: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl(2-(phosphonooxy)benzyl)carbamate (37)

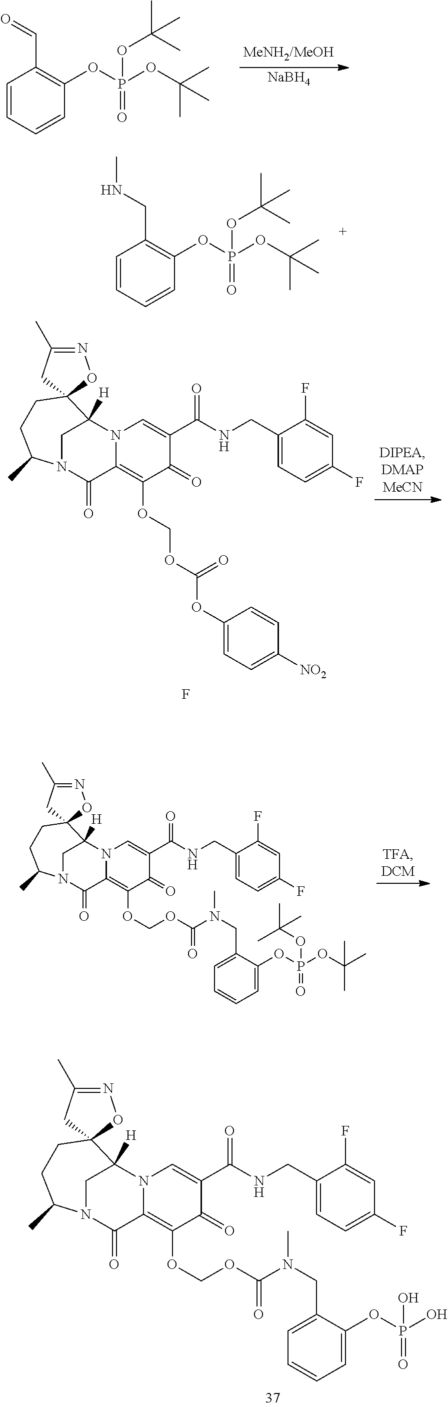

### Step 1: Preparation of di-tert-butyl (2-((methylamino)methyl)phenyl) phosphate

Di-tert-butyl (2-formylphenyl) phosphate (1.98 g, 6.3 mmol) was dissolved in MeOH (30 mL) followed by addition of a methylamine in MeOH solution (235 mg, 7.56 mmol) at room temperature. After 30 min, the reaction was cooled to 0° C. followed by addition of sodium borohydride (286 mg, 7.56 mmol). After 1 h at rt, the reaction was quenched by adding water. The reaction mixture was extracted with ethyl acetate and washed with brine. After drying with anhydrous MgSO₄, the solvent was removed and the residue was purified by column chromatography on silica gel to provide title product. MS (m/z) 329.858 [M+H]⁺.

### Step 2: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-((di-tert-butoxyphosphoryl)oxy)benzyl)(methyl)carbamate

Into a mixture of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate F, 150 mg, 0.22 mmol) and di-tert-butyl (2-((methylamino)methyl)phenyl) phosphate (109 mg, 0.33 mmol) in MeCN (10 mL) was added DIPEA (42.7 mg, 0.33 mmol) and 4-dimethylaminopyridine (53.8 mg, 0.033 mmol) at rt. After 2 h, the reaction mixture was extracted with ethyl acetate and washed with brine. After drying with anhydrous MgSO₄, the solvent was removed and the residue was purified by column chromatography on silica gel to provide title product. MS (m/z) 871.843 [M+H]⁺.

### Step 3: Preparation of ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl(2-(phosphonooxy)benzyl)carbamate (37)

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl(2-(phosphonooxy)benzyl)carbamate was synthesized followed the same procedure as Example 32, except using (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-((di-tert-butoxyphosphoryl)oxy)benzyl)(methyl)carbamate instead of 2-((di-tert-butoxyphosphoryl)oxy)benzyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate in Step 3. MS (m/z) 759.924 [M+H]⁺. ¹H NMR (400 MHz, DMSO-d6) δ 10.37-10.30 (m, 1H), 8.69 (d, J=12.9 Hz, 1H), 7.42 (d, J=7.3 Hz, 1H), 7.26 (dd, J=8.2, 2.2 Hz, 3H), 7.20-7.01 (m, 3H), 5.78 (dd, J=49.0, 6.3 Hz, 1H), 5.63 (d, J=6.3 Hz, 1H), 4.62 (s, 2H), 4.55 (t, J=5.2 Hz, 2H), 4.50-4.37 (m, 2H), 3.66 (d, J=9.8 Hz, 2H), 3.00 (d, J=17.5 Hz, 1H), 2.77 (d, J=13.4 Hz, 3H), 2.62 (dd, J=17.6, 7.6 Hz, 1H), 1.94 (d, J=2.4 Hz, 3H), 1.77 (dd, J=22.0, 10.2 Hz, 3H), 1.27 (dd, J=13.9, 8.1 Hz, 1H), 1.13-1.04 (m, 3H).

### Example 38: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl methyl(2-(phosphonooxy)benzyl)carbamate (38)

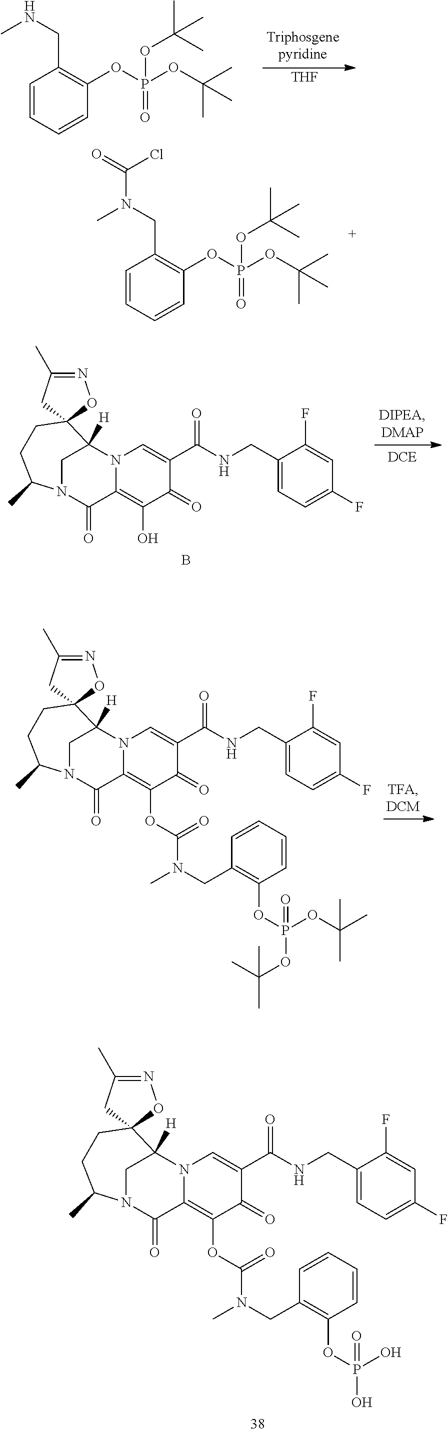

(3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl methyl(2-(phosphonooxy)benzyl)carbamate was synthesized following the same procedure as Steps 2-3 of Example 1, except starting from di-tert-butyl (2-((methylamino)methyl)phenyl) phosphate, prepared in Example 37 instead of di-tert-butyl ((2-(methylamino)pyridin-3-yl)methyl) phosphate. MS (m/z) 729.883 [M+H]+. 1H NMR (400 MHz, DMSO-d6) δ 10.30-10.12 (n, 1H), 8.81 (s, 1H), 7.43 (dd, J=11.0, 4.7 Hz, 2H), 7.36-7.21 (m, 3H), 7.21-7.11 (m, 1H), 7.13-7.03 (m, 1H), 4.69 (s, 2H), 4.55 (dt, J=15.6, 7.3 Hz, 3H), 3.70 (s, 2H), 3.39 (q, J=7.2 Hz, 2H), 3.03 (d, J=18.0 Hz, 2H), 2.84 (s, 1H), 2.71 (s, 1H), 1.95 (s, 3H), 1.82 (s, 3H), 1.24 (s, 1H), 1.18 (d, J=6.6 Hz, 3H).

### Example 39: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl(4-(phosphonooxy)benzyl)carbamate (39)

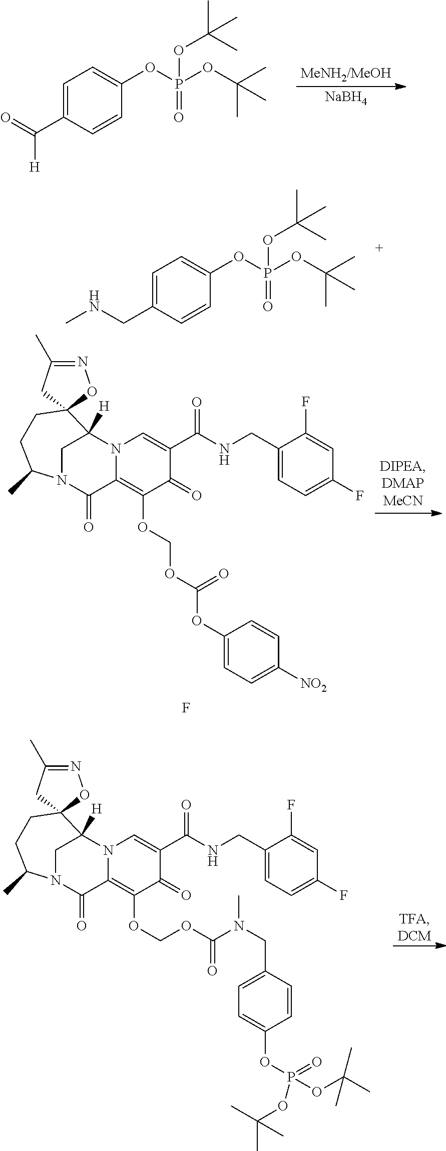

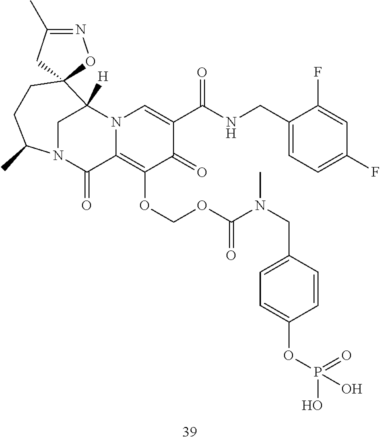 [SMILES: CC1=NO[C@@]2(CC[C@H](C)N3C[C@H]2n2cc(C(=O)NCc4ccc(F)cc4F)c(=O)c(OCOC(=O)N(C)Cc4ccc(OP(=O)(O)O)cc4)c2C3=O)C1]

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl(4-(phosphonooxy)benzyl)carbamate was synthesized followed the same procedure as Example 37, except starting from di-tert-butyl (4-formylphenyl) phosphate instead of di-tert-butyl (2-formylphenyl) phosphate in Step 1. MS (m/z) 759.985 [M+H]⁺. ¹H NMR (400 MHz, DMSO-d6) δ 10.33 (d, J=5.6 Hz, 1H), 8.68 (d, J=7.7 Hz, 1H), 7.42 (d, J=7.7 Hz, 1H), 7.25 (tt, J=7.1, 3.1 Hz, 3H), 7.18-7.01 (m, 3H), 5.91-5.70 (m, 1H), 5.70-5.58 (m, 1H), 4.62 (s, 2H), 4.56 (d, J=5.9 Hz, 2H), 4.50-4.37 (m, 1H), 4.34-4.24 (m, 1H), 3.65 (s, 2H), 3.02 (s, 1H), 2.72 (d, J=8.9 Hz, 3H), 2.64 (s, 1H), 1.94 (s, 3H), 1.75 (d, J=15.5 Hz, 3H), 1.32 (s, 1H), 1.08 (dt, J=13.5, 6.8 Hz, 3H).

### Example 40: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl methyl(4-(phosphonooxy)benzyl)carbamate (40)

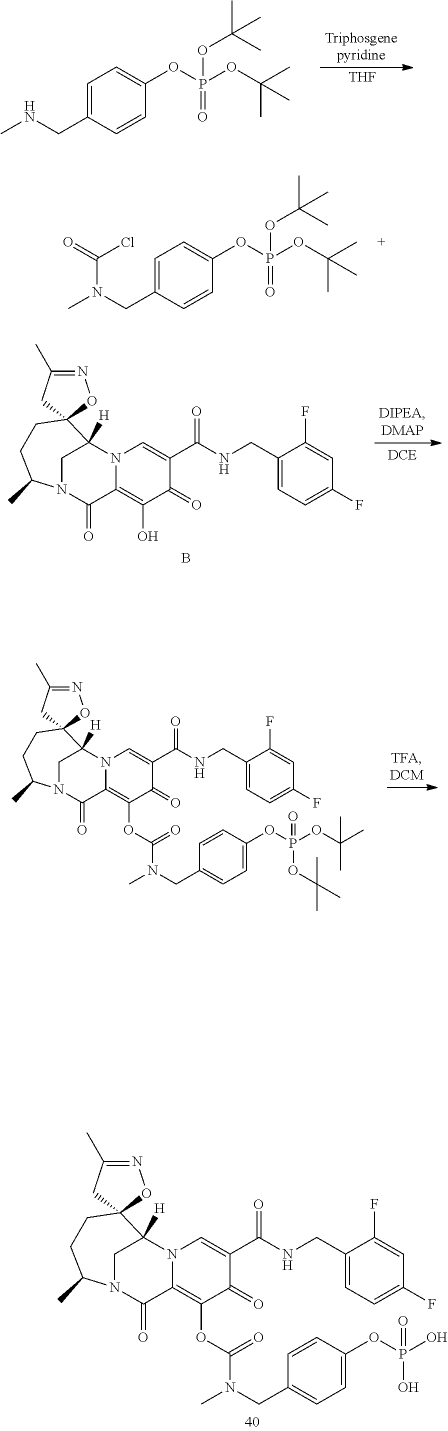

(3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl methyl(4-(phosphonooxy)benzyl)carbamate was synthesized following the same procedure as Steps 2-3 of Example 1, except starting from di-tert-butyl (4-((methylamino)methyl)phenyl) phosphate instead of di-tert-butyl ((2-(methylamino)pyridin-3-yl)methyl) phosphate. MS (m/z) 729.931 [M+H]⁺. ¹H NMR (400 MHz, DMSO-d6) δ 10.24 (d, J=6.3 Hz, 1H), 8.80 (s, 1H), 7.60-7.40 (m, 2H), 7.39-7.23 (m, 2H), 7.20-7.00 (m, 3H), 4.80-4.44 (m, 5H), 3.72 (s, 2H), 3.39 (d, J=7.0 Hz, 2H), 3.05-2.93 (m, 2H), 2.78 (s, 1H), 2.70 (d, J=8.3 Hz, 1H), 1.96 (s, 3H), 1.83 (d, J=9.4 Hz, 3H), 1.24 (s, 1H), 1.18 (d, J=6.6 Hz, 3H).

### Example 41: Synthesis of 4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl)phenyl dihydrogen phosphate (41)

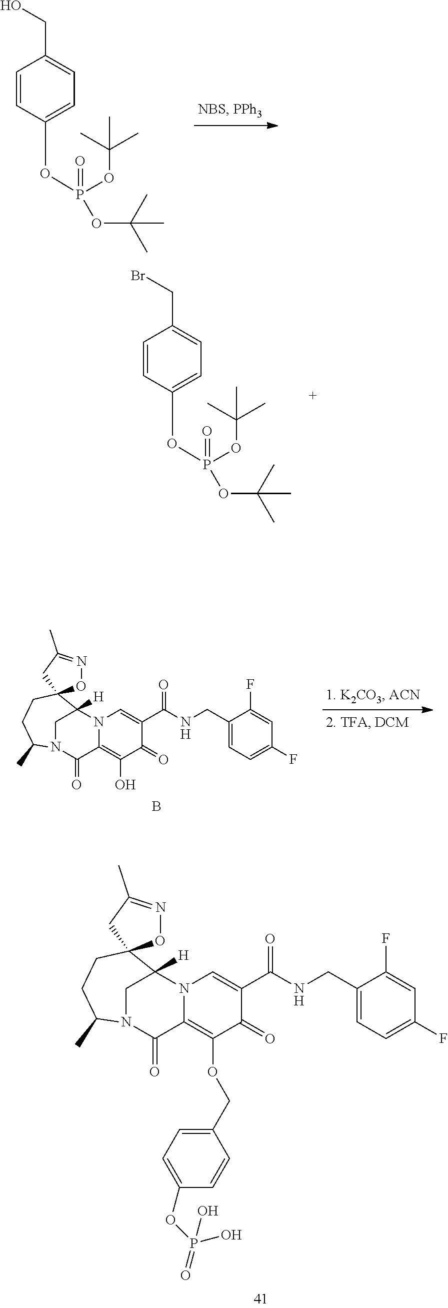

### Step 1: Synthesis of 4-(bromomethyl)phenyl di-tert-butyl phosphate

To a solution of di-tert-butyl (4-(hydroxymethyl)phenyl) phosphate (144 mg, 0.46 mmol), PPh₃ (119 mg, 0.46 mmol) in 3 mL dry CH₂Cl₂, was added N-bromosuccinimide (NBS, 85.1 mg, 0.48 mmol) in several portions at 0° C. The mixture was stirred for overnight at room temperature. After that, the solid was filtered through celite. The solvent was removed by vacuum, and the residue was purified by column chromatography to obtain the title compound. MS (m/z) 380.2 [M+H]+.

### Step 2: Synthesis of 4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl)phenyl dihydrogen phosphate (41)

To a solution of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (intermediate B, 100 mg, 0.21 mmol) and 4-(bromomethyl)phenyl di-tert-butyl phosphate (85.8 mg, 0.23 mmol) in 3 mL of ACN, was added potassium bicarbonate (42.6 mg, 0.31 mmol). The reaction mixture was heated to 70° C. for 2 hours. Filtered through celite and concentrated. The crude product obtained was then dissolved in DCM (1.0 mL) at 0° C., and treated with TFA (0.4 mL) in DCM (0.6 mL) slowly. The mixture was then removed from cooling bath after addition and stirred at room temperature for 4 h. The mixture was concentrated and dissolved in DMF. The mixture was purified by reverse phase preparative HPLC (5-100% MeCN/H₂O w/ 0.1% TFA) to afford the title compound. MS (m/z) 673.1 [M+H]+. 1H NMR (400 MHz, Methanol-d4) δ 8.51 (s, 1H), 7.54-7.35 (m, 3H), 7.29-7.12 (m, 2H), 7.11-6.89 (m, 2H), 5.30 (d, J=10.6 Hz, 1H), 5.16 (d, J=10.6 Hz, 1H), 4.74-4.56 (m, 2H), 4.38 (s, 1H), 3.72 (dd, J=15.3, 1.9 Hz, 1H), 3.47 (dd, J=15.2, 2.7 Hz, 1H), 3.21-3.02 (m, 1H), 2.68 (dd, J=17.9, 1.2 Hz, 1H), 2.05 (t, J=0.9 Hz, 3H), 1.99-1.81 (m, 3H), 1.24 (d, J=6.7 Hz, 3H).

### Example 42: Preparation of (2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetyl)-L-glutamic acid (42)

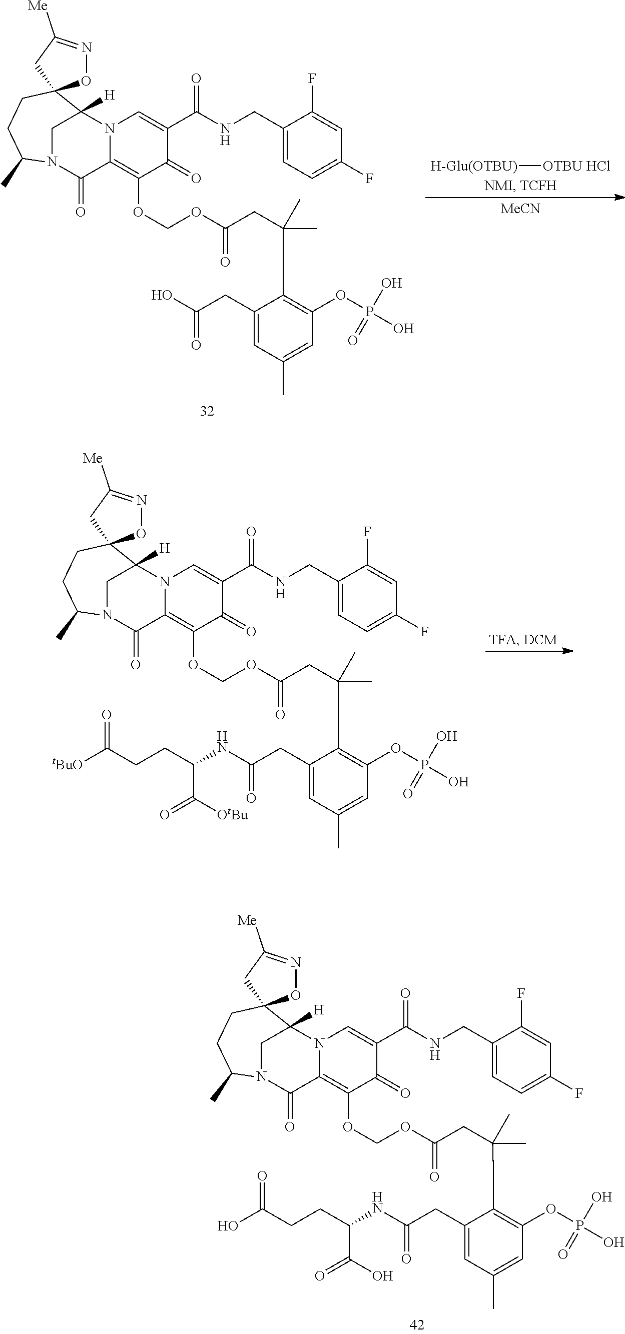

### Step 1: Preparation di-tert-butyl (2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetyl)-L-glutamate

To a mixture of 2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetic acid (Example 32, 0.1 g, 0.118 mmol) and H-Glu(OtBu)-OtBu HCl (0.046 g, 0.16 mmol) in ACN (3 mL) at 0° C. was added N-methylimidazole (0.024 g, 0.30 mmol) followed by TCFH (0.040 g, 0.142 mmol). The mixture was stirred at 0° C. for 30 minutes and then stirred at room temperature for 30 minutes. Then it was concentrated and the resulting residue was diluted with EtOAc and washed with water. The aqueous layer was extracted with EtOAc (3×) and the combined organic layer was washed with a 1:1 solution of brine/water and brine. The organic phase was dried over Na₂SO₄, filtered, concentrated and purified by silica gel chromatography. MS (m/z) 1086.3 [M+H]+.

### Step 2: Preparation (2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetyl)-L-glutamic acid (42)

The title compound was made following the same method as Example 23 Step 3, except using di-tert-butyl (2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetyl)-L-glutamate instead of tert-butyl 2-(3-((di-tert-butoxyphosphoryl)oxy)-2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methylphenyl)acetate. MS (m/z) 974.2 [M+H]+. 1H NMR (400 MHz, DMSO-d6) δ 10.29 (t, J=5.9 Hz, 1H), 8.67 (s, 1H), 8.14 (d, J=7.7 Hz, 1H), 7.59-7.32 (m, 2H), 7.32-7.15 (m, 1H), 7.15-7.02 (m, 2H), 6.66 (s, 1H), 5.69 (d, J=6.3 Hz, 1H), 5.45 (d, J=6.2 Hz, 1H), 4.94 (d, J=7.6 Hz, 1H), 4.62 (d, J=7.8 Hz, 2H), 4.54 (t, J=5.8 Hz, 2H), 4.27-4.11 (m, 1H), 3.84-3.69 (m, 2H), 3.68 (s, 2H), 3.06-2.86 (m, 3H), 2.70-2.59 (m, 2H), 2.36-2.25 (m, 3H), 2.15 (s, 3H), 1.95 (s, 3H), 1.77 (d, J=11.5 Hz, 3H), 1.46 (d, J=2.6 Hz, 5H), 1.15 (d, J=6.7 Hz, 3H).

### Example 43: Preparation of (2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetyl)-L-aspartic acid (43)

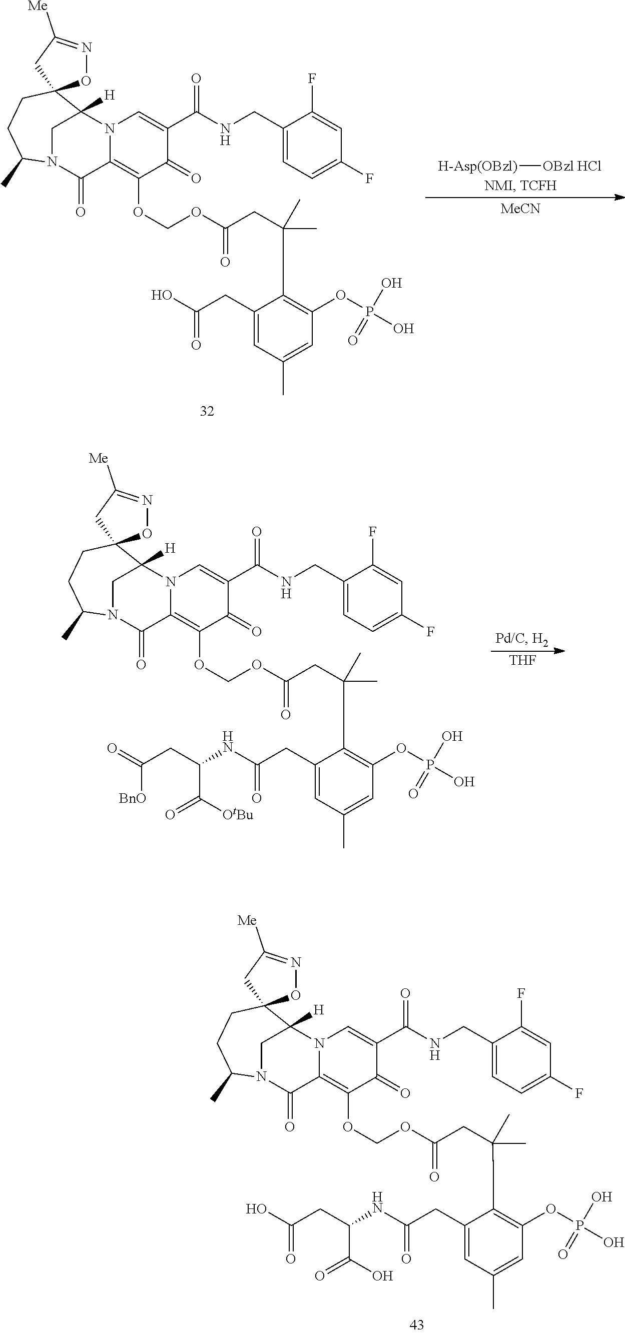

### Step 1: Preparation dibenzyl (2-(2-(4-((((3′S,5S,7′R)-10′-((2,4-difluorobenzyl)carbamoyl)-3,3′-dimethyl-1′,11′-dioxo-1′,4′,5′,11′I-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetyl-L-aspartate

To a mixture of 2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetic acid (Example 32, 0.1 g, 0.118 mmol) and H-Asp(OBzl)-OBzl HCl (0.050 g, 0.14 mmol) in ACN (3 mL) at 0° C. was added N-methylimidazole (0.024 g, 0.30 mmol) followed by TCFH (0.040 g, 0.142 mmol). The mixture was stirred at 0° C. for 30 minutes and then stirred at room temperature for 30 minutes. Then it was concentrated and the resulting residue was diluted with EtOAc and washed with water. The aqueous layer was extracted with EtOAc (3×) and the combined organic layer was washed with a 1:1 solution of brine/water and brine. The organic phase was dried over Na₂SO₄, filtered, concentrated and purified by silica gel chromatography. MS (m/z) 1141.2 [M+H]+.

### Step 2: Preparation (2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetyl)-L-aspartic acid (43)

The title compound was made following the same method as Example 19 Step 3, except using dibenzyl (2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetyl)-L-aspartate instead of 4-((bis(benzyloxy)phosphoryl)oxy)benzyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate. MS (m/z) 960.1 [M+H]+. 1H NMR (400 MHz, DMSO-d6) δ 10.29 (t, J=6.0 Hz, 11H), 8.67 (s, 11H), 8.11 (d, J=8.2 Hz, 1H), 7.54-7.33 (m, 1H), 7.33-7.18 (m, 1H), 7.18-6.97 (m, 2H), 6.59 (d, J=46.8 Hz, 2H), 5.70 (d, J=6.2 Hz, 1H), 5.45 (d, J=6.2 Hz, 1H), 4.71-4.44 (m, 5H), 3.73 (s, 1H), 3.67 (s, 2H), 3.07-2.91 (m, 2H), 2.75-2.61 (m, 3H), 2.16 (d, J=5.1 Hz, 3H), 1.95 (s, 3H), 1.90-1.67 (m, 3H), 1.45 (s, 6H), 1.28 (dd, J=23.7, 10.8 Hz, 1H), 1.15 (d, J=6.6 Hz, 3H).

### Example 44: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((1S,2S)-2-(phosphonooxy)cyclohexyl) carbonate (44)

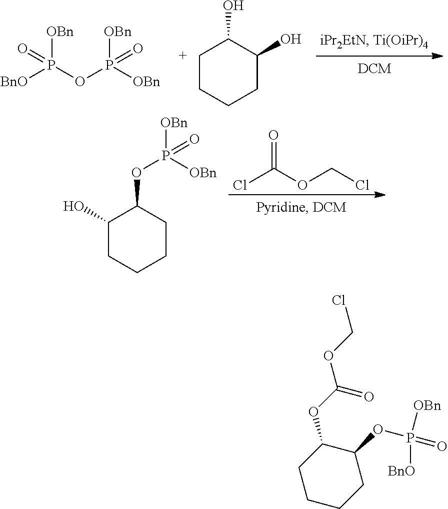

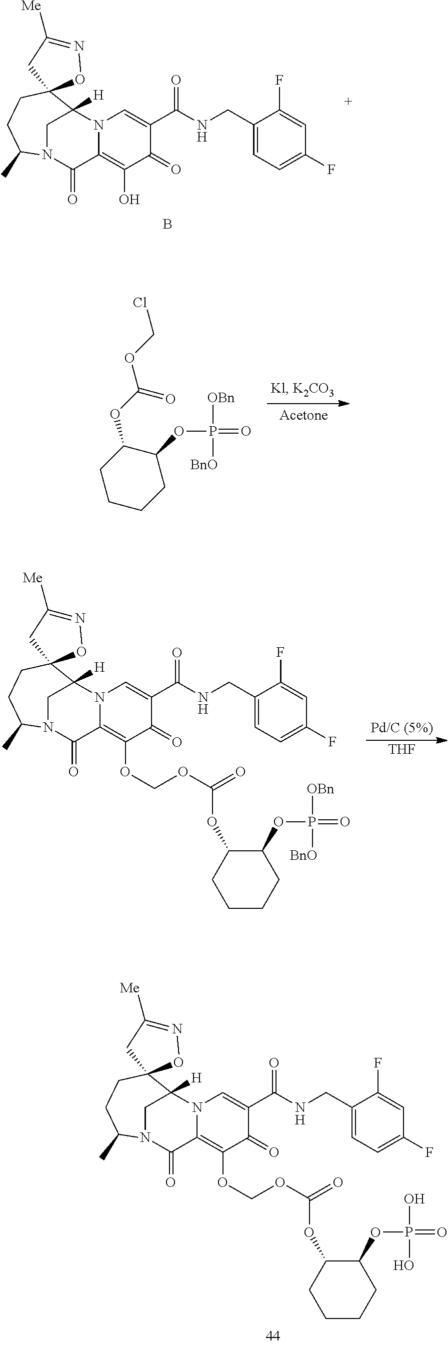

### Step 1: Preparation of dibenzyl ((1S,2S)-2-hydroxycyclohexyl) phosphate

To the solution of (1S,2S)-cyclohexane-1,2-diol (1.0 g, 8.61 mmol) was added N,N-diisopropylethylamine (2.25 ml, 12.9 mmol) followed by dibenzyl dibenzyloxyphosphoryl phosphate (2.78 g, 5.17 mmol) and titanium(IV) isopropoxide (0.245 g, 0.861 mmol) sequentially. The resulting mixture was stirred at room temperature for 4 hrs. Then the reaction mixture was filtered through a pad of silica gel/magnesium sulfate mixture (20:1), the solid was rinsed with ethyl acetate/hexanes (75%, 50 ml). The filtrate was dried over sodium sulfate, filtered, concentrated under reduced pressure. The residue was purified by normal phase chromatography on a silica gel (eluting with 0-100% EtOAc) to afford the title compound. MS (m/z) 377.2 [M+H]⁺.

### Step 2: Preparation of (1S,2S)-2-((bis(benzyloxy)phosphoryl)oxy)cyclohexyl (chloromethyl) carbonate

Dibenzyl ((1S,2S)-2-hydroxycyclohexyl) phosphate (2.1 g, 5.58 mmol) was added to a stirred mixture of chloromethyl chloroformate (0.563 ml, 0.614 mmol) in dichloromethane (20 mL) then followed by the addition of pyridine (0.14 ml, 1.87 mmol) drop-wisely at room temperature. The reaction was stirred for overnight before it was diluted with DCM and washed sequentially with HCl (0.5M) and water. The organic layer was dried over magnesium sulfate, filtered and evaporate to obtain the title compound, and used directly without further purification. MS (m/z) 469.1 [M+H]+.

### Step 3: Preparation of (1S,2S)-2-((bis(benzyloxy)phosphoryl)oxy)cyclohexyl ((((3′S,5S,7′R)-10′-((2,4-difluorobenzyl)carbamoyl)-3,3′,7′-trimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To the solution of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.1 g, 0.206 mmol) in Acetone (2 ml) was added KI (0.0444 g, 0.267 mmol) and K₂CO₃ (0.0426 g, 0.308 mmol) followed by (1S,2S)-2-((bis(benzyloxy)phosphoryl)oxy)cyclohexyl (chloromethyl) carbonate (0.193 g, 0.411 mmol). The resulting mixture was left to stir at room temperature for overnight and then was concentrated. The residue was purified by silica gel column chromatography (0-100% EtOAc/Hexane) to afford the title compound. MS (m/z) 919.7 [M+H]+.

### Step 4: Preparation of (((3′S,5S,7′R)-10′-((2,4-difluorobenzyl)carbamoyl)-3,3′,7′-trimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((1S,2S)-2-(phosphonooxy)cyclohexyl) carbonate (44)

To the solution of (1S,2S)-2-((bis(benzyloxy)phosphoryl)oxy)cyclohexyl ((((3′S,5S,7′R)-10′-((2,4-difluorobenzyl)carbamoyl)-3,3′,7′-trimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (0.189 g, 0.206 mmol) in THF (15 ml) was added Pd/C (5 wt %) (0.0219 g, 0.0206 mmol). The resulting mixture was purged with hydrogen three times before it was stirred under hydrogen for 1 h. The reaction was then filtered through Celite®, and concentrated. The resulting residue was purified by reverse phase preparative HPLC (5-100% MeCN/H₂O w/ 0.1% TFA) to afford the title compound. MS (m/z) 739.2 [M+H]+. ¹H NMR (400 MHz, DMSO) δ 10.28 (t, J=6.0 Hz, 1H), 8.70 (s, 1H), 7.50-7.36 (m, 1H), 7.34-7.19 (m, 1H), 7.19-7.02 (m, 1H), 5.84-5.68 (m, 2H), 4.71-4.49 (m, 5H), 4.20-4.02 (m, 2H), 3.70-3.67 (m, 2H), 3.57 (s, 2H), 3.00 (d, J=17.6 Hz, 1H), 2.66 (d, J=17.5 Hz, 1H), 1.95 (s, 4H), 1.88-1.72 (m, 3H), 1.65-1.49 (m, 2H), 1.50-1.36 (m, 2H), 1.35-1.18 (m, 3H), 1.15 (d, J=6.7 Hz, 3H).

### Example 45: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((1R,2R)-2-(phosphonooxy)cyclohexyl) carbonate (45)

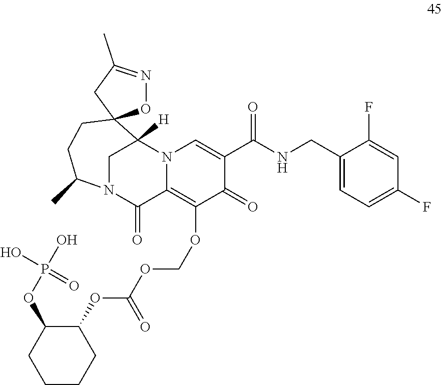 [SMILES: CC1=NO[C@@]2(CC[C@H](C)N3C[C@H]2n2cc(C(=O)NCc4ccc(F)cc4F)c(=O)c(OCOC(=O)O[C@@H]4CCCC[C@H]4OP(=O)(O)O)c2C3=O)C1]

The title compound was made following the same method as Example 44, except in step 1, (1S,2S)-cyclohexane-1,2-diol was replaced by (1R,2R)-cyclohexane-1,2-diol. MS (m/z) 739.2 [M+H]+. ¹H NMR (400 MHz, DMSO) δ 10.29 (t, J=5.9 Hz, 1H), 8.71 (s, 1H), 7.51-7.35 (m, 1H), 7.35-7.21 (m, 1H), 7.18-6.99 (m, 1H), 5.97-5.83 (m, 1H), 5.66-5.48 (m, 1H), 4.70-4.48 (m, 7H), 4.21-4.08 (m, 1H), 3.77-3.71 (m, 1H), 3.73-3.62 (m, 3H), 3.00 (d, J=17.5 Hz, 1H), 2.63 (d, J=17.5 Hz, 1H), 1.95 (s, 4H), 1.86-1.72 (m, 3H), 1.66-1.35 (m, 3H), 1.34-1.20 (m, 3H), 1.16 (d, J=6.6 Hz, 3H).

### Example 46: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((1S,2S)-2-(phosphonooxy)cyclopentyl) carbonate (46)

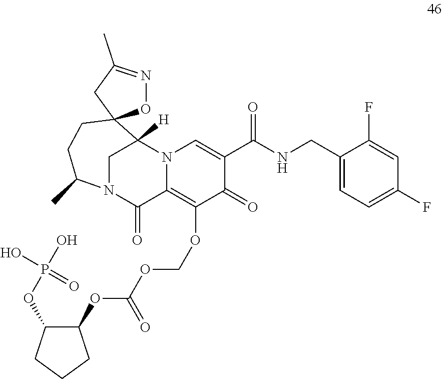 [SMILES: CC1=NO[C@@]2(CC[C@H](C)N3C[C@H]2n2cc(C(=O)NCc4ccc(F)cc4F)c(=O)c(OCOC(=O)O[C@H]4CCC[C@@H]4OP(=O)(O)O)c2C3=O)C1]

The title compound was made following the same method as Example 44, except in step 1, (1S,2S)-cyclohexane-1,2-diol was replaced by (1S,2S)-cyclopentane-1,2-diol. MS (m/z) 725.9 [M+H]+.

¹H NMR (400 MHz, DMSO) δ 10.27 (t, J=6.0 Hz, 1H), 8.71 (s, 1H), 7.53-7.31 (m, 1H), 7.31-7.19 (m, 1H), 7.19-6.97 (m, 1H), 5.80 (d, J=6.5 Hz, 1H), 5.68 (d, J=6.6 Hz, 1H), 4.94-4.79 (m, 2H), 4.79-4.37 (m, 6H), 3.80-3.57 (m, 2H), 3.00 (d, J=17.5 Hz, 1H), 2.67 (d, J=17.5 Hz, 1H), 2.04-1.49 (m, 12H), 1.41-1.20 (m, 1H), 1.16 (d, J=6.7 Hz, 3H).

### Example 47: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((1-((phosphonooxy)methyl)cyclobutyl)methyl) carbonate (47)

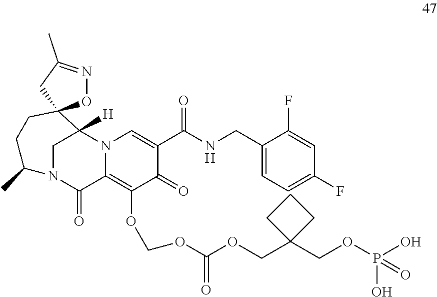 [SMILES: CC1=NO[C@@]2(CC[C@H](C)N3C[C@H]2n2cc(C(=O)NCc4ccc(F)cc4F)c(=O)c(OCOC(=O)OCC4(COP(=O)(O)O)CCC4)c2C3=O)C1]

The title compound was made following the same method as Example 44, except in step 1, (1S,2S)-cyclohexane-1,2-diol was replaced by cyclobutane-1,1-diyldimethanol. And in step 3 (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide was replaced by (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide. MS (m/z) 738.93 [M+H+]. ¹H NMR (400 MHz, CDCl₃) δ 10.44 (t, J=6.0 Hz, 114), 8.56 (s, 114), 7.43-7.31 (m, 11H), 6.93-6.72 (m, 2H), 5.85 (d, J=6.6 Hz, 1H), 5.74 (d, J=6.6 Hz, 114), 4.91-4.74 (m, 114), 4.74-4.52 (m, 2H), 4.37-3.96 (m, 5H), 3.93-3.67 (m, 2H), 3.09 (d, J=17.9 Hz, 1H), 2.59 (d, J=17.9 Hz, 2H), 2.14-1.80 (m, 13H), 1.60-1.46 (m, 1H), 1.26 (d, J=6.7 Hz, 3H). z

### Example 48: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((1-((phosphonooxy)methyl)cyclobutyl)methyl) carbonate (48)

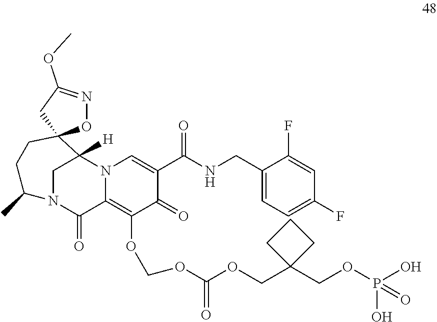 [SMILES: COC1=NO[C@@]2(CC[C@H](C)N3C[C@H]2n2cc(C(=O)NCc4ccc(F)cc4F)c(=O)c(OCOC(=O)OCC4(COP(=O)(O)O)CCC4)c2C3=O)C1]

The title compound was made following the same method as Example 44, except in step 1, (1S,2S)-cyclohexane-1,2-diol was replaced by cyclobutane-1,1-diyldimethanol and in Step 4 (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide instead of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide. MS (m/z) 754.9 [M+H]+. ¹H NMR (400 MHz, CDCl₃) δ 10.28 (t, J=6.0 Hz, 1H), 8.58 (s, 1H), 7.44-7.34 (m, 1H), 6.93-6.61 (m, 2H), 5.95 (d, J=6.7 Hz, 1H), 5.79 (d, J=6.7 Hz, 1H), 4.90-4.80 (m, 4H), 4.72-4.54 (m, 1H), 4.41 (s, 1H), 4.29-4.20 (m, 1H), 4.18-4.11 (m, 1H), 4.09-3.96 (m, 2H), 3.91 (s, 3H), 3.81-3.73 (m, 2H), 3.04 (d, J=17.0 Hz, 1H), 2.69 (d, J=17.1 Hz, 1H), 2.14-1.76 (m, 9H), 1.76-1.47 (m, 1H), 1.23-1.20 (m, 3H).

### Example 49: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (((1R,2S)-2-((phosphonooxy)methyl)cyclopropyl)methyl) carbonate (49)

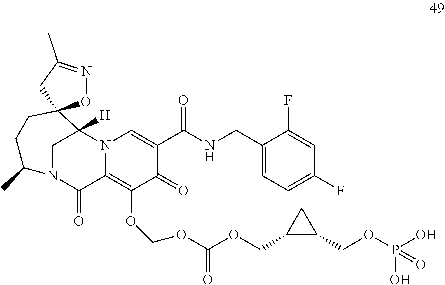 [SMILES: CC1=NO[C@@]2(CC[C@H](C)N3C[C@H]2n2cc(C(=O)NCc4ccc(F)cc4F)c(=O)c(OCOC(=O)OC[C@@H]4C[C@@H]4COP(=O)(O)O)c2C3=O)C1]

The title compound was made following the same method as Example 44, except in step 1, (1S,2S)-cyclohexane-1,2-diol was replaced by [(1S,2R)-2-(hydroxymethyl)cyclopropyl]methanol. MS (m/z) 724.83 [M+H]+I. ¹H NMR (400 MHz, CDCl₃) δ 10.44 (s, 1H), 8.55-8.39 (m, 1H), 7.44-7.31 (m, 1H), 6.91-6.67 (m, 2H), 5.90-5.64 (m, 1H), 5.43-4.72 (m, 9H), 4.69-4.60 (m, 2H), 4.43-4.27 (m, 1H), 4.29-4.18 (m, 1H), 4.10-3.98 (m, 1H), 3.90-3.65 (m, 2H), 3.09 (d, J=17.9 Hz, 1H), 2.63 (d, J=17.8 Hz, 1H), 1.98-1.84 (m, 3H), 1.70-1.49 (m, 1H), 1.46-1.31 (m, 2H), 1.30-1.17 (m, 3H), 1.04-0.84 (m, 1H), 0.39 (s, 1H).

### Intermediates G and H: Preparation of (1R,2S)-2-((bis(benzyloxy)phosphoryl)oxy)cyclohexyl (chloromethyl) carbonate (Intermediate G) and (1S,2R)-2-((bis(benzyloxy)phosphoryl)oxy)cyclohexyl (chloromethyl) carbonate (Intermediate H)

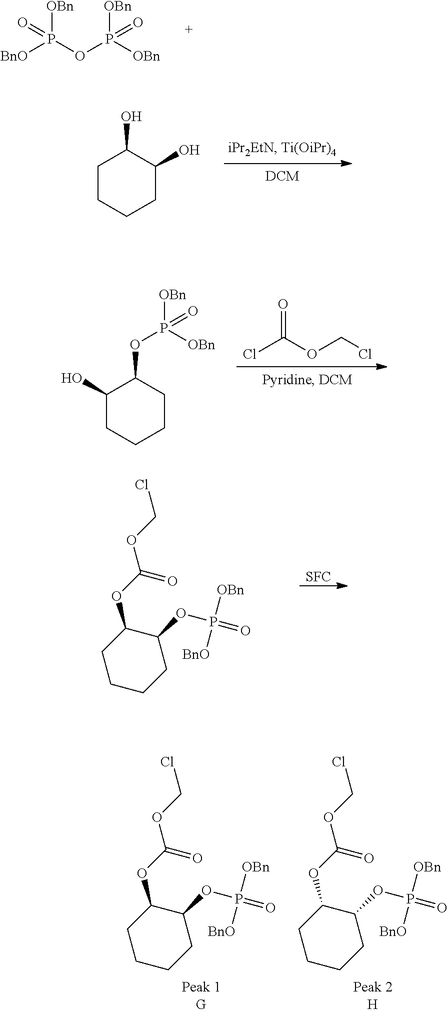

### Step 1: Preparation of dibenzyl ((cis-2-hydroxycyclohexyl) phosphate

To the solution of meso-cyclohexane-1,2-diol (1.0 g, 8.61 mmol) was added N,N-diisopropylethylamine (2.25 ml, 12.9 mmol) followed by dibenzyl dibenzyloxyphosphoryl phosphate (2.78 g, 5.17 mmol) and titanium(IV) isopropoxide (0.245 g, 0.861 mmol) sequentially. The resulting mixture was stirred at room temperature for 4 hrs. Then the reaction mixture was filtered through a pad of silica gel/magnesium sulfate mixture (20:1), the solid was rinsed with ethyl acetate/hexanes (75%, 50 ml). The filtrate was dried over sodium sulfate, filtered, concentrated under reduced pressure. The residue was purified by normal phase chromatography on a silica gel (0-100% EtOAc in Hexanes) to afford the title compound. MS (m/z) 377.2 [M+H]+.

### Step 2: Preparation of cis-2-((bis(benzyloxy)phosphoryl)oxy)cyclohexyl (chloromethyl) carbonate

Dibenzyl ((1S,2R)-2-hydroxycyclohexyl) phosphate (0.850 g, 2.26 mmol) was added to a stirred mixture of chloromethyl chloroformate (0.22 mL, 2.48 mmol) in dichloromethane (10 mL) then followed by the addition of pyridine (0.228 ml, 2.82 mmol) drop-wisely at room temperature. The reaction was stirred for overnight before it was diluted with DCM and washed sequentially with HCl (0.5M) and water. The organic layer was dried over magnesium sulfate, filtered and evaporate to obtain title compound. MS (m/z) 469.8 [M+H]+.

### Step 3: Preparation of (1R,2S)-2-((bis(benzyloxy)phosphoryl)oxy)cyclohexyl (chloromethyl) carbonate (Intermediate G, Peak 1) and (1S,2R)-2-((bis(benzyloxy)phosphoryl)oxy)cyclohexyl (chloromethyl) carbonate (Intermediate H, Peak 2)

SFC separation of cis-2-((bis(benzyloxy)phosphoryl)oxy)cyclohexyl (chloromethyl) carbonate (Column: AD-H, 5 μm 21×250 mm 60 mL/min, 15% MeOH)

Intermediate G (Peak 1): (1R,2S)-2-((bis(benzyloxy)phosphoryl)oxy)cyclohexyl (chloromethyl) carbonate: MS (m/z)=469.8 [M+H]+

Intermediate H (Peak 2): (1S,2R)-2-((bis(benzyloxy)phosphoryl)oxy)cyclohexyl (chloromethyl) carbonate: MS (m/z)=469.8 [M+H]+

### Example 50: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((1R,2S)-2-(phosphonooxy)cyclohexyl) carbonate (50)

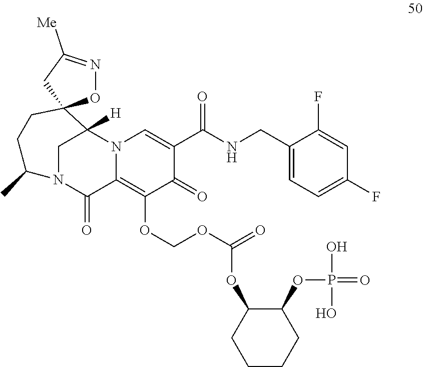 [SMILES: CC1=NO[C@@]2(CC[C@H](C)N3C[C@H]2n2cc(C(=O)NCc4ccc(F)cc4F)c(=O)c(OCOC(=O)O[C@@H]4CCCC[C@@H]4OP(=O)(O)O)c2C3=O)C1]

The title compound was made following the same method as Example 44, except in step 3, (1S,2S)-2-((bis(benzyloxy)phosphoryl)oxy)cyclohexyl (chloromethyl) carbonate was replaced by Intermediate G. MS (m/z) 739.0 [M+H]+. 1H NMR (400 MHz, DMSO) δ 10.27 (t, J=6.0 Hz, 1H), 8.71 (s, 1H), 7.48-7.36 (m, 1H), 7.31-7.18 (m, 1H), 7.15-7.00 (m, 1H), 5.81-5.77 (m, 1H), 5.73-5.67 (m, 1H), 4.77 (s, 1H), 4.70-4.45 (m, 4H), 4.26 (s, 1H), 3.71-3.66 (m, 4H), 3.01 (d, J=17.5 Hz, 1H), 2.65 (d, J=17.4 Hz, 1H), 1.95 (s, 3H), 1.87-1.62 (m, 6H), 1.58-1.39 (m, 2H), 1.33-1.28 (m, 4H), 1.15 (d, J=6.6 Hz, 3H).

### Example 51: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((1S,2R)-2-(phosphonooxy)cyclohexyl) carbonate (51)

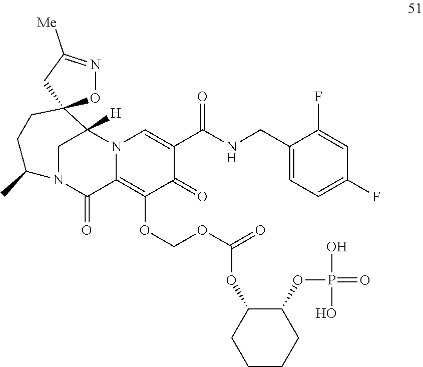 [SMILES: CC1=NO[C@@]2(CC[C@H](C)N3C[C@H]2n2cc(C(=O)NCc4ccc(F)cc4F)c(=O)c(OCOC(=O)O[C@H]4CCCC[C@H]4OP(=O)(O)O)c2C3=O)C1]

The title compound was made following the same method as Example 44, except in step 3, (1S,2S)-2-((bis(benzyloxy)phosphoryl)oxy)cyclohexyl (chloromethyl) carbonate was replaced by Intermediate H. MS (m/z) 739.1 [M+H]+. 1H NMR (400 MHz, DMSO) δ 10.40-10.17 (m, 1H), 8.70 (s, 1H), 7.52-7.35 (m, 1H), 7.35-7.16 (m, 1H), 7.16-6.95 (m, 1H), 5.90 (d, J=6.4 Hz, 1H), 5.57 (d, J=6.4 Hz, 1H), 4.81-4.48 (m, 5H), 4.31-4.17 (m, 1H), 3.79-3.60 (m, 4H), 3.00 (d, J=17.5 Hz, 1H), 2.74-2.55 (m, 1H), 1.95 (s, 3H), 1.88-1.23 (m, 12H), 1.16 (d, J=6.7 Hz, 3H).

### Example 52: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (((1S,2S)-2-(phosphonooxy)cyclohexyl)methyl) carbonate (52)

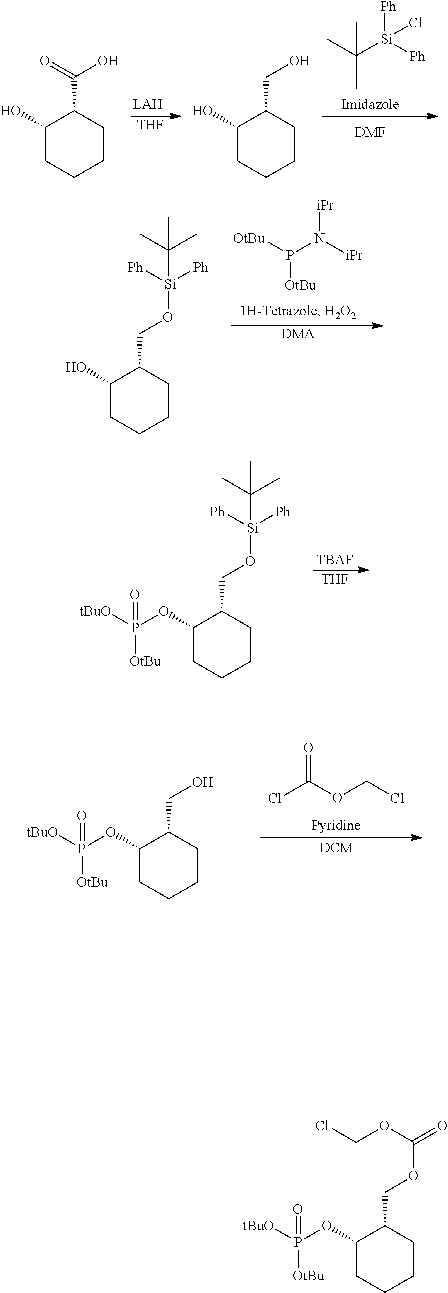

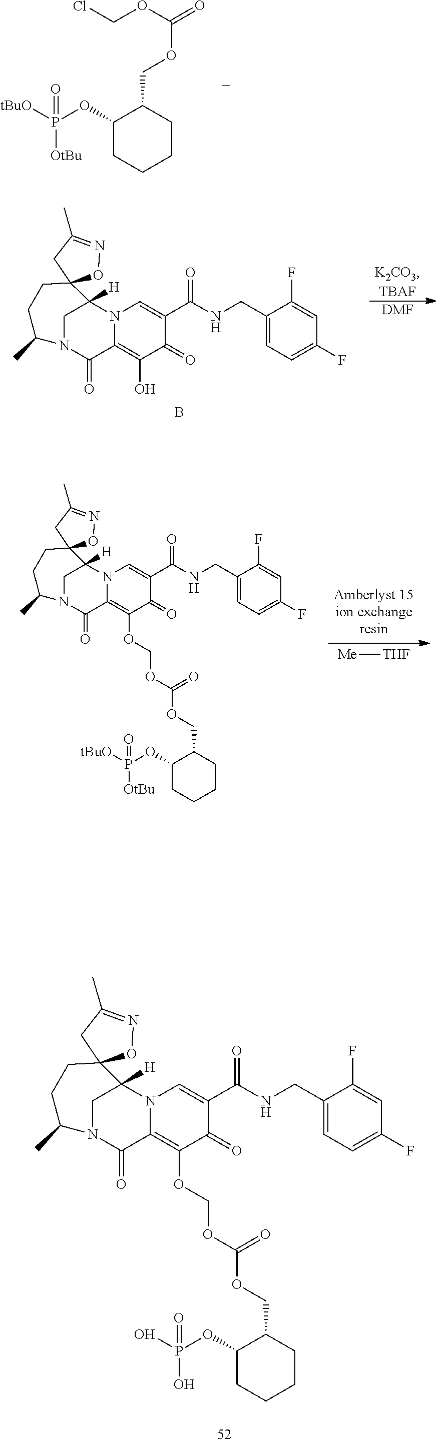

### Step 1: Preparation of (1S,2S)-2-(hydroxymethyl)cyclohexan-1-ol

(1R,2S)-2-Hydroxycyclohexane-1-carboxylic acid (2.5 g, 17.34 mmol) was dissolved in tetrahydrofuran (60 mL) under argon atmosphere. The solution was cooled to 0° C. with a water/ice bath. Lithium aluminum hydride (2.3 M in THF) (12.7 mL, 29.5 mmol) dropwise to the mixture with evolution of gas. The reaction mixture was stirred at 0° C. for a further 15 min. The reaction mixture was then heated at 70° C. for 17 h. Then reaction mixture was cooled to 0° C. with ice-water bath. Na₂SO₄·10 H₂O (55 g) was added portion-wise. The resulting mixture was stirred at room temperature for 2 h. The slurry was filtered through celite plug. The filter top was washed with THF (3×20 mL). The filtrate was collected and concentrated to afford the title compound. MS (m/z) 131.00 [M+H]⁺.

### Step 2: Preparation of (1S,2S)-2-(((tert-butyldiphenylsilyl)oxy)methyl)cyclohexan-1-ol

(1S,2S)-2-(hydroxymethyl)cyclohexan-1-ol (0.98 g, 7.38 mmol) was dissolved in DMF at room temperature under argon atmosphere. Imidazole (0.66 g, 9.69 mmol) was added. Then TBDPS-Cl (2.03 g, 7.38 mmol) was added dropwise. The resulting reaction mixture was stirred at room temperature for 17 h. Reaction mixture was then diluted with EtOAc (20 mL). Treated with saturated ammonium chloride (20 ml), extracted with EtOAc (20 mL) and washed with brine (10 mL). The reaction mixture was dried, filtered, concentrated, and purified by silica gel chromatography (0-100% EtOAc in Hexanes) to provide the title compound. ¹H NMR (400 MHz, CD3CN) δ 7.79-7.66 (m, 4H), 7.53-7.37 (m, 6H), 4.07-4.02 (m, 1H), 3.74 (dd, J=9.9, 6.8 Hz, 1H), 3.60 (dd, J=9.9, 6.2 Hz, 1H), 2.60 (d, J=3.9 Hz, 1H), 1.75-1.25 (m, 9H), 1.06 (s, 9H).

### Step 3: Preparation of di-tert-butyl ((1S,2S)-2-(((tert-butyldiphenylsilyl)oxy)methyl)cyclohexyl) phosphate

To a solution of (1S,2S)-2-(((tert-butyldiphenylsilyl)oxy)methyl)cyclohexan-1-ol (0.546 g, 1.43 mmol) and 1H-tetrazole (0.25 g, 3.56 mmol) in N,N-dimethylacetamide (20 mL) was added di-tert-butyl diisopropylphosphoramidite (0.611 g, 2.14 mmol) at room temperature and stirred at room temperature for 24 h. Another portion of 1H-tetrazole (0.5 g, 7.12 mmol) and di-tert-butyl diisopropylphosphoramidite (1.22 g, 4.28 mmol) were added. The reaction mixture was stirred at room temperature for an additional 17 h. The slurry was then cooled down to 0° C. H₂O₂ (50%, 3.32 g, 34.2 mmol) was added dropwise and the reaction was warmed to room temperature for 3 h. The reaction mixture was then diluted with EtOAc (20 mL) and was washed with water (20 mL) and brine (10 mL), dried, filtered, concentrated, and purified by silica gel chromatography (0-100% EtOAc) to afford the title compound. MS (m/z) 583.20 [M+Na]⁺.

### Step 4: Preparation of di-tert-butyl ((1S,2S)-2-(hydroxymethyl)cyclohexyl) phosphate

Di-tert-butyl ((1S,2S)-2-(((tert-butyldiphenylsilyl)oxy)methyl)cyclohexyl) phosphate (0.64 g, 1.12 mmol) was dissolved in Me-THF (6.3 mL) under argon atmosphere and the solution was cooled down to 0° C. TBAF (1 M in THF, 1.68 mL, 1.68 mmol) was added dropwise. The reaction mixture was stirred for 3 h. Reaction mixture was cooled to 0° C., and a second portion of TBAF (1 M in THF, 1.68 mL, 1.68 mmol) was added dropwise and the reaction mixture was stirred for an additional 3 h. Reaction mixture was then diluted with EtOAc, washed with water and brine dried, filtered, concentrated, and purified by silica gel chromatography (0-100% EtOAc) to afford the title compound. ¹H NMR (400 MHz, CD3CN) S 4.67 (dt, J=8.3, 2.8 Hz, 1H), 4.16 (dd, J=9.1, 5.2 Hz, 1H), 3.43 (ddd, J=11.6, 9.8, 5.0 Hz, 1H), 3.32 (ddd, J=11.6, 8.9, 5.7 Hz, 1H), 2.03 (dtd, J=11.3, 3.8, 1.8 Hz, 1H), 1.77-1.51 (m, 5H), 1.49 (dd, J=5.6, 0.7 Hz, 18H), 1.42-1.13 (m, 3H).

### Step 5: Preparation of chloromethyl (((1S,2S)-2-((di-tert-butoxyphosphoryl)oxy(cyclohexyl) methyl) carbonate

di-tert-Butyl ((1S,2S)-2-(hydroxymethyl)cyclohexyl) phosphate (227 mg, 0.859 mmol) was dissolved in DCM (8 mL) at 0° C. Pyridine (136 mg, 1.72 mmol) was added. And then a solution of chloromethyl chloroformate (0.115 mg, 1.29 mmol) in DCM (0.5 mL) was added and reaction mixture was stirred for 17 h, washed with water, and saturated ammonium chloride, dried, and concentrated to afford the title compound. MS (m/z) 414.87 [M+H]

### Step 6: Preparation of ((1S,2S)-2-((di-tert-butoxyphosphoryl)oxy)cyclohexyl)methyl ((((3′S,5S,7′R)-10′-((2,4-difluorobenzyl)carbamoyl)-3,3′-dimethyl-1,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

(((1S,2S)-2-((di-tert-butoxyphosphoryl)oxy)cyclohexyl)methyl) carbonate (190 mg, 0.458 mmol) and (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 130 mg, 0.267 mmol) were mixed with DMF (2.5 mL) at room temperature. Potassium carbonate (102 mg, 0.739 mmol) and tetrabutylammonium iodide (99 mg, 0.267 mmol) were added sequentially and reaction mixture was stirred at 60° C. for 4 h and then room temperature overnight. The reaction mixture was then diluted with EtOAc (20 mL), washed with water (10 mL) and brine (10 mL). The reaction mixture was then, dried, filtered, concentrated and purified by silica gel chromatography (0-100% EtOAc) to afford the title compound. MS (m/z) 864.52 [M+H]⁺.

### Step 7: Preparation of product (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (((1S,2S)-2-(phosphonooxy)cyclohexyl)methyl) carbonate (52)

((1S,2S)-2-((Di-tert-butoxyphosphoryl)oxy)cyclohexyl)methyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (58 mg, 0.0613 mmol) was dissolved in Me-THF (12.2 mL) at room temperature. Amberlyst 15, ion exchange resin (183 mg, 0.582 mmol) was added. The reaction mixture was vigorously stirred at room temperature for 3 h. The second portion of Amberlyst 15 ion exchange resin (183 mg, 0.582 mmol) was added. The reaction was continued with vigorous stirring for 17 h. The resin was removed by filtration and washed with THF (3×10 mL). The combined filtrate was collected and was concentrated to afford the title compound. MS (m/z) 752.90 [M+H]⁺. ¹H NMR (400 MHz, DMSO) δ 10.31-10.20 (m, 1H), 8.71 (s, 1H), 7.51-7.31 (m, 1H), 7.31-7.15 (m, 1H), 7.12-7.00 (m, 1H), 5.80 (d, J=6.5 Hz, 1H), 5.68 (d, J=6.5 Hz, 1H), 4.69-4.58 (m, 2H), 4.58-4.52 (m, 1H), 4.49-4.43 (m, 2H), 4.40-4.34 (m, 1H), 4.06-3.98 (m, 1H), 3.69 (s, 1H), 3.42-3.32 (m, 2H), 3.26-3.15 (m, 2H), 3.06-2.96 (m, 1H), 1.97-1.89 (m, 5H), 1.86-1.30 (m, 14H).

### Example 53: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((1S,2S)-2-((phosphonooxy)methyl)cyclohexyl) carbonate (53)

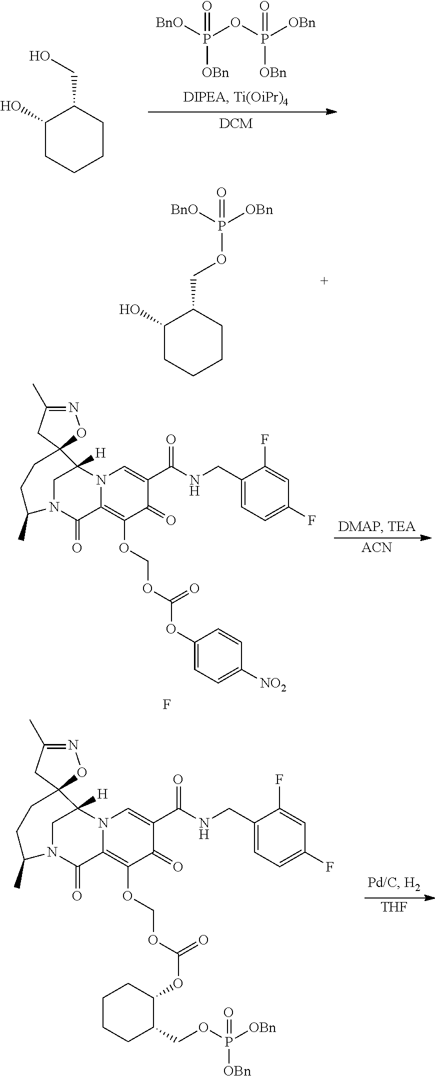

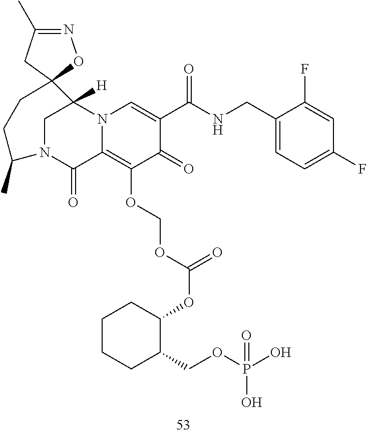 [SMILES: CC1=NO[C@@]2(CC[C@H](C)N3C[C@H]2n2cc(C(=O)NCc4ccc(F)cc4F)c(=O)c(OCOC(=O)O[C@H]4CCCC[C@H]4COP(=O)(O)O)c2C3=O)C1]

### Step 1: Preparation of (((1S,2S)-2-hydroxycyclohexyl)methyl) phosphate

To a solution of tetrabenzyl diphosphate (5.1 g, 9.19 mmol) and (1S,2S)-2-(hydroxymethyl)cyclohexan-1-ol (1.13 g, 8.51 mmol) in dichloromethane (110 mL) were added N,N-diisopropylethylamine (4.11 mL, 22.9 mmol) and titanium(IV) isopropoxide (0.444 g, 1.56 mmol). The mixture was stirred at room temperature for 17 h. The reaction mixture was concentrated and was purified by column chromatography on a silica gel column (0-100% EtOAc) to afford the title compound. MS (m/z) 390.96 [M+H⁺].

### Step 2: Preparation of (S,2S)-2-(((bis(benzyloxy)phosphoryl)oxy)methyl)cyclohexyl ((((3′S, 5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

(((3′S,5S,7′R)-10′-((2,4-Difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate F, 200 mg, 0.293 mmol) was dissolved in acetonitrile (5 mL) at room temperature under argon atmosphere. Triethylamine (0.286 mL, 2.05 mmol) and DMAP (7.2 mg, 0.0587 mmol) were added sequentially. Then a solution of dibenzyl (((1S,2S)-2-hydroxycyclohexyl)methyl) phosphate (0.687 g, 1.76 mmol) in ACN (2 ml) was added dropwise over 15 min and the reaction mixture was stirred at room temperature for 24 h. The reaction mixture was diluted with EtOAc (10 mL) and washed with saturated ammonium chloride in water, dried, filtered concentrated and purified by silica gel chromatography to afford a mixture of two regio-isomers. The material was then taken up in acetonitrile (5 mL) and was purified on reverse phase preparative chromatography (5-100% MeCN/H2O) to afford the title compound. MS (m/z) 932.85 [M+H]⁺.

### Step 3: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-v)oxy)methyl ((1S,2S)-2-((phosphonooxy)methyl)cyclohexyl) carbonate (53)

((((3′S,5S,7′R)-10′-((2,4-Difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (76 mg, 0.083 mmol) was dissolved in THF (10 mL) at room temperature. Palladium on activated carbon (5%) (20 mg, 0.0083 mmol) was added under H₂ balloon. The vacuum-fill process was taken 3 times, followed by bubbling of hydrogen gas for 7 min. The reaction mixture was then stirred at room temperature under H₂ atmosphere for 1 hr. The reaction mixture was filtered through syringe filter and washed with THF (10 mL). The combined filtrate was concentrated to dryness to provide the title compound. MS (m/z) 752.88 [M+H]⁺. ¹H NMR (400 MHz, DMSO) δ 10.28 (t, J=6.0 Hz, 1H), 8.71 (s, 1H), 7.47-7.37 (m, 1H), 7.30-7.20 (m, 1H), 7.13-7.03 (m, 1H), 5.84-5.63 (m, 2H), 4.95-4.43 (m, 5H), 3.95-3.53 (m, 12H), 3.01 (d, J=17.5 Hz, 1H), 2.65 (d, J=17.7 Hz, 1H), 1.95 (s, 3H), 1.69-1.57 (m, 6H), 1.57-1.48 (m, 1H), 1.16-1.13 (m, 3H).

### Example 54: (((3′S,5S,7′R)-10′-((2,4-difluorobenzyl)carbamoyl)-3,3′-dimethyl-1′11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((cis)-3-(phosphonooxy)cyclopentyl) carbonate (54)

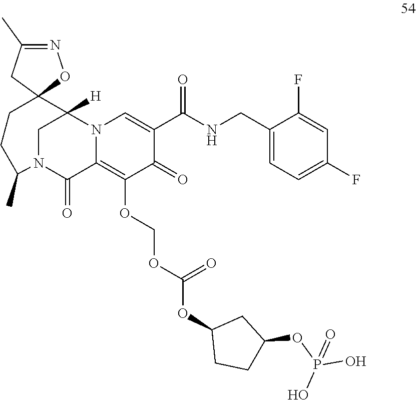 [SMILES: CC1=NO[C@@]2(CC[C@H](C)N3C[C@H]2n2cc(C(=O)NCc4ccc(F)cc4F)c(=O)c(OCOC(=O)O[C@@H]4CC[C@H](OP(=O)(O)O)C4)c2C3=O)C1]

The title compound was made following the same method as Example 53, except in step 1, (1S,2S)-2-(hydroxymethyl)cyclohexan-1-ol was replaced by (cis)-cyclopentane-1,3-diol. MS (m/z) 724.84 [M+H]⁺. ¹H NMR (400 MHz, DMSO) δ 10.36-10.14 (m, 1H), 8.83-8.64 (m, 1H), 7.51-7.34 (m, 1H), 7.34-7.21 (m, 1H), 7.11-7.02 (m, 1H), 5.89-5.72 (m, 1H), 5.72-5.57 (m, 1H), 4.99-4.77 (m, 1H), 4.77-4.42 (m, 5H), 3.73-3.65 (m, 2H), 3.00 (d, J=17.5 Hz, 1H), 2.64 (d, J=17.6 Hz, 1H), 1.95 (s, 3H), 1.91-1.77 (m, 10H), 1.36-1.22 (m, 2H), 1.19-1.13 (m, 3H).

### Intermediate I: Preparation of dibenzyl [4-(hydroxymethyl)-3-methyl-2-oxo-oxazolidin-4-yl]methyl phosphate (G)

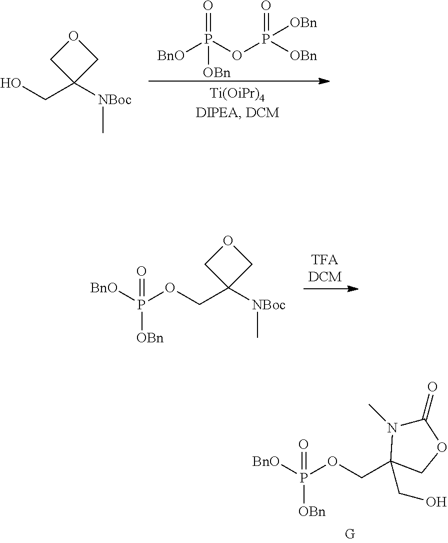 [SMILES: CN(C(=O)OC(C)(C)C)C1(CO)COC1 ; O=P(OCc1ccccc1)(OCc1ccccc1)OP(=O)(OCc1ccccc1)OCc1ccccc1 ; CN(C(=O)OC(C)(C)C)C1(COP(=O)(OCc2ccccc2)OCc2ccccc2)COC1 ; CN1C(=O)OCC1(CO)COP(=O)(OCc1ccccc1)OCc1ccccc1]

### Step 1: Preparation of tert-butyl (3-(((bis(benzyloxy)phosphoryl)oxy)methyl)oxetan-3-yl)(methyl)carbamate

tert-butyl (3-(hydroxymethyl)oxetan-3-yl)(methyl)carbamate (468 mg, 2.15 mmol) in DCM (5 mL), dibenzyl dibenzyloxyphosphoryl phosphate (1.16 g, 2.15 mmol), DIEPA (0.94 mL, 5.4 mmol) and tetraisopropoxytitanium (61.2 mg, 0.215 mmol) was added to the mixture. The reaction mixture was stirring vigorously for 16 h. The organic layer was then separated and washed with NaHCO₃ solution and dried over anhydrous MgSO₄. After removing the solvent, the residue was concentrated and purified by flash column (30 to 90)% EtOAc/Hexanes) to get title compound. MS (m/z) 478.0. [M+H]+. 1H NMR (400 MHz, Chloroform-d) δ 7.37 (s, 10H), 5.17-4.99 (m, 4H), 4.67 (d, J=6.7 Hz, 2H), 4.35 (ddd, J=43.2, 36.3, 6.0 Hz, 4H), 2.69 (d, J=5.9 Hz, 3H), 1.41 (d, J=12.0 Hz, 9H).

### Step 2: Preparation of dibenzyl (4-(hydroxymethyl)-3-methyl-2-oxo-oxazolidin-4-yl methyl phosphate

To a stirred solution of tert-butyl N-[3-(dibenzyloxyphosphoryloxymethyl)oxetan-3-yl]-N-methyl-carbamate (0.1 g, 0.21 mmol) in DCM (1.5 mL) and 0.375 mL of TFA at 0° C. was added to the mixture. The mixture was stirred for 1 h at 0° C. The reaction mixture was diluted with DCM and washed with saturated sodium bicarbonate solution and water. The organic layer was dried over anhydrous Na₂SO₄, filtered, and concentrated under reduced pressure to get the title compound. MS (m/z) 422.1 [M+H]+.

### Example 55: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((3-methyl-2-oxo-4-((phosphonooxy)methyl)oxazolidin-4-yl)methyl) carbonate (55)

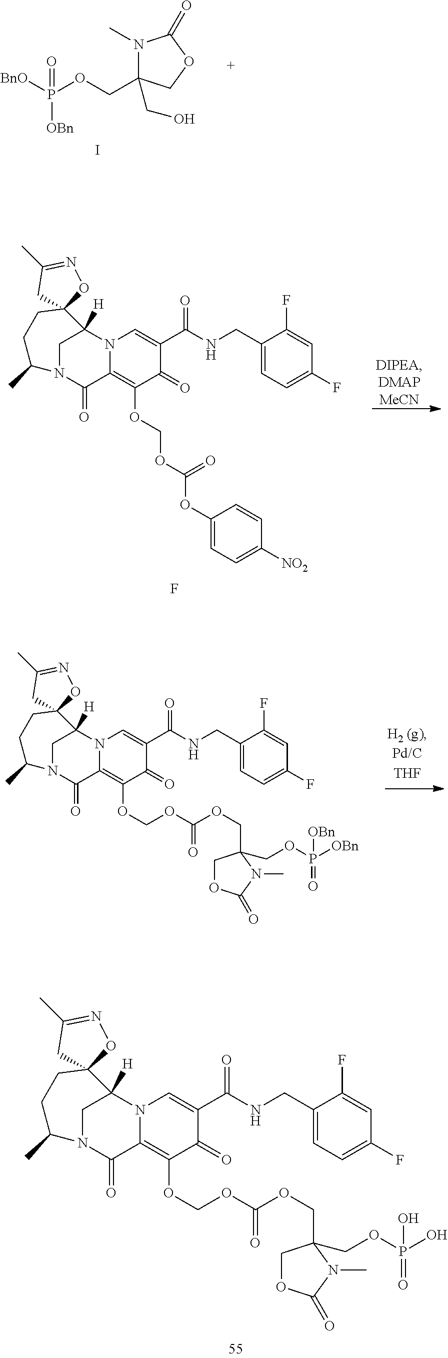

### Step 1: Preparation of (4-(((bis(benzyloxy)phosphoryl)oxy)methyl)-3-methyl-2-oxooxazolidin-4-yl)methyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

Into a mixture of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate F, 113 mg, 0.166 mmol) and dibenzyl [4-(hydroxymethyl)-3-methyl-2-oxo-oxazolidin-4-yl]methyl phosphate (Intermediate I, 70 mg, 0.166 mmol) in MeCN (2 mL) was added triethylamine (16.8 mg, 0.166 mmol) at rt. After 4 hi, the reaction mixture was extracted with ethyl acetate and washed with brine. After drying with anhydrous MgSO₄, the solvent was removed, and the residue was was purified by column chromatography on silica gel (0-100% EtOAc) to provide title product. MS (m/z) 964.3 [M+H]+.

### Step 2: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((3-methyl-2-oxo-4-((phosphonooxy)methyl)oxazolidin-4-yl)methyl) carbonate (55)

To a solution of (4-(((bis(benzyloxy)phosphoryl)oxy)methyl)-3-methyl-2-oxooxazolidin-4-yl)methyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (61.4 mg, 0.064 mmol) in 5 mL of THF was added 7 mg of 10% palladium on carbon. The flask was evacuated and backfilled with hydrogen gas (2×), then sparged with hydrogen for 2 min. The reaction mixture was left to stir under a hydrogen balloon atmosphere for 2 h. The reaction mixture was filtered, rinsed with THF, and concentrated to afford a residue, which was purified by reverse phase prep HPLC (5-100% MeCN/water) to afford the title compound. MS (m/z) 784.1 ¹H NMR (400 MHz, MeOD) δ 8.58 (s, 1H), 7.56-7.37 (m, 1H), 7.04-6.91 (m, 3H), 6.07-5.90 (m, 1H), 5.90-5.74 (m, 1H), 4.88-4.72 (m, 2H), 4.71-4.58 (m, 2H), 4.50-4.15 (m, 7H), 4.15-3.98 (m, 1H), 3.93-3.66 (m, 2H), 3.23-3.08 (m, 1H), 2.95-2.82 (m, 3H), 2.72 (d, J=17.9 Hz, 1H), 2.07 (s, 3H), 2.02-1.87 (m, 3H), 1.62-1.47 (m, 1H), 1.26 (d, J=6.7 Hz, 3H).

### Intermediate J: Preparation of dibenzyl ((3-(hydroxymethyl)oxetan-3-yl)methyl) phosphate

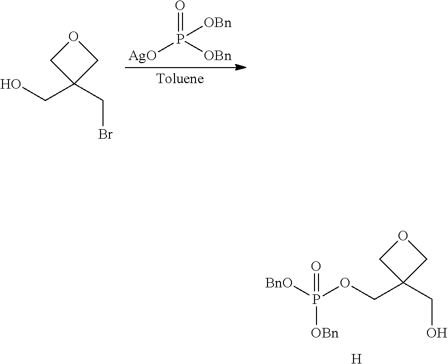

To a stirred solution of [3-(bromomethyl)oxetan-3-yl]methanol (0.5 g, 2.76 mmol) in toluene (5 mL) at room temperature under argon was added silver dibenzylphosphate (1.06 g, 2.76 mmol) under argon. The mixture was stirred at reflux for 16 h. The reaction mixture was allowed to cool to rt and was filtered, rinsing the solids with toluene (5V). The filtrate was concentrated under reduced pressure and purified by silica gel column chromatography (30-50% EtOAc/Hexanes) to afford the title compound. MS (m/z) 379.2 [M+H]⁺.

### Example 56: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((3-((phosphonooxy)methyl)oxetan-3-yl)methyl) carbonate (56)

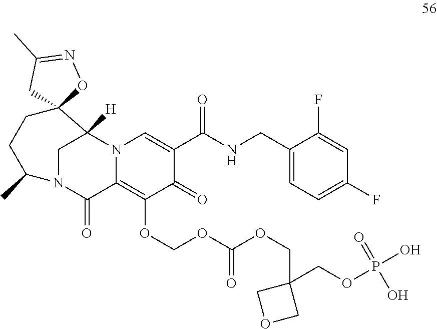 [SMILES: CC1=NO[C@@]2(CC[C@H](C)N3C[C@H]2n2cc(C(=O)NCc4ccc(F)cc4F)c(=O)c(OCOC(=O)OCC4(COP(=O)(O)O)COC4)c2C3=O)C1]

The title compound was made following the same method as Example 55, except using dibenzyl [3-(hydroxymethyl)oxetan-3-yl]methyl phosphate (Intermediate J) instead of dibenzyl [4-(hydroxymethyl)-3-methyl-2-oxo-oxazolidin-4-yl]methyl phosphate (Intermediate 1). MS (m/z) 741.0 [M+H]+. ¹H NMR (400 MHz, MeOD) δ 8.58 (s, 1H), 7.51-7.36 (m, 1H), 7.05-6.87 (m, 2H), 5.97 (d, J=6.6 Hz, 1H), 5.77 (d, J=6.6 Hz, 1H), 4.87-4.71 (m, 1H), 4.73-4.61 (m, 2H), 4.61-4.53 (m, 4H), 4.53-4.36 (m, 3H), 4.33-4.15 (m, 2H), 3.88-3.70 (m, 2H), 3.16 (d, J=17.8 Hz, 1H), 2.71 (d, J=17.9 Hz, 1H), 2.06 (s, 3H), 2.02-1.85 (m, 3H), 1.63-1.47 (m, 1H), 1.42 (s, 1H), 1.33-1.20 (m, 4H), 0.91 (d, J=9.4 Hz, 1H).

### Example 57: Preparation of (2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetyl)-D-proline (57)

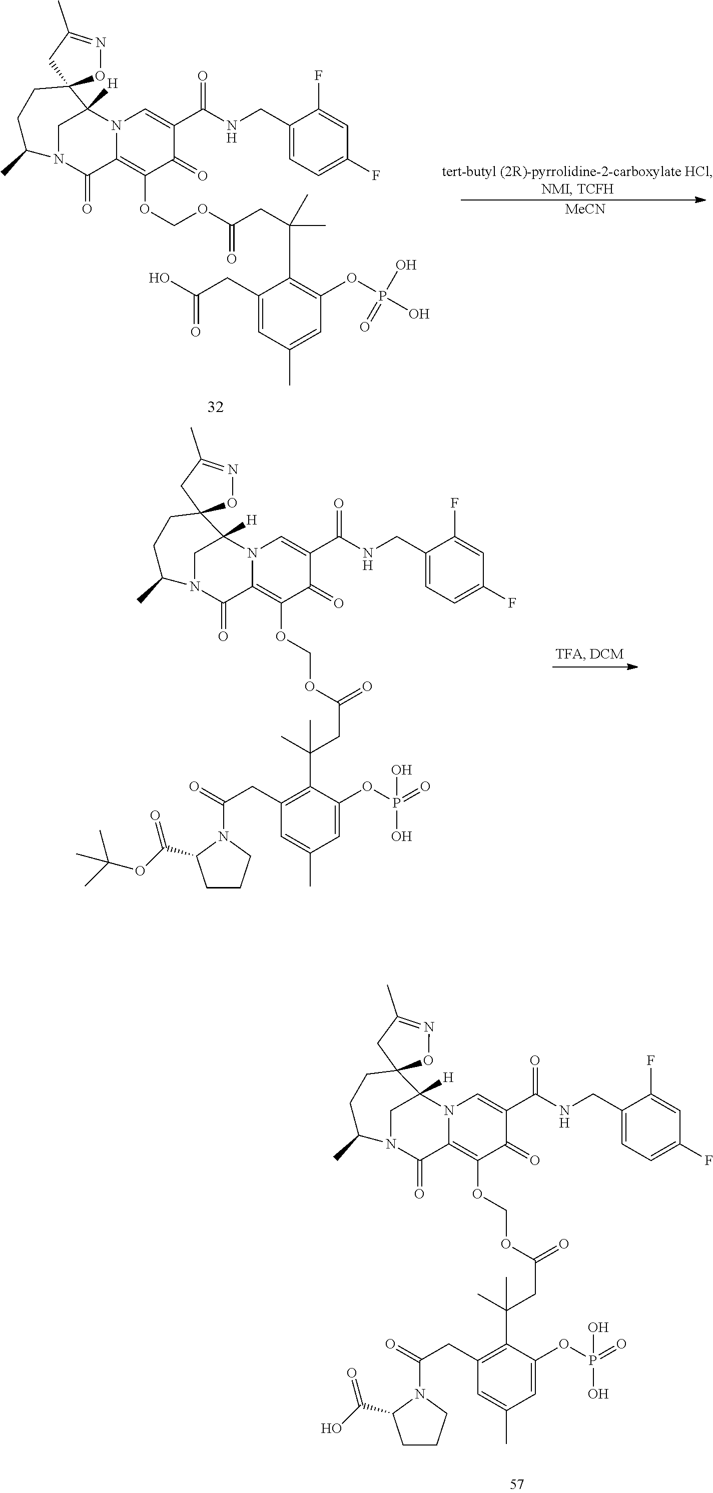 [SMILES: "MeCN" ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)O)cc(C)cc4OP(=O)(O)O)c2C3=O)C1 ; "tert-butyl (2R)-pyrrolidine-2-carboxylate HCl,NMI, TCFH" ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)N5CCC[C@@H]5C(=O)OC(C)(C)C)cc(C)cc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)N5CCC[C@@H]5C(=O)O)cc(C)cc4OP(=O)(O)O)c2C3=O)C1]

### Step 1: Preparation of tert-butyl (2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy-2-methyl-4-oxobutan-2-yl)-5-methyl-3-(phosphonooxy)phenyl)acetyl)-D-prolinate

To a mixture of 2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetic acid (Example 32, 55 mg, 0.065 mmol) and tert-butyl (2R)-pyrrolidine-2-carboxylate HCl (20 mg, 0.097 mmol) in ACN (2 mL) at 0° C. was added N-methylimidazole (19.8 mg, 0.19 mmol) followed by TCFH (21 mg, 0.078 mmol). The mixture was stirred at 0° C. for 30 minutes and then stirred at room temperature for 30 minutes. Then it was concentrated and the resulting residue was purified by silica gel chromatography (0-100% EtOAc) to afford the title compound. MS (m/z) 997.7 [M+H]⁺.

### Step 2: Preparation of (2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetyl)-D-proline (57)

The title compound was made following the same method as Example 23 Step 3, except using tert-butyl (2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetyl)-D-prolinate instead of tert-butyl 2-(3-((di-tert-butoxyphosphoryl)oxy)-2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methylphenyl)acetate. MS (m/z) 941.7 [M+H]1. ¹H NMR (400 MHz, MeOD) δ 8.55 (d, J=4.0 Hz, 1H), 7.50-7.39 (m, 1H), 7.26-7.11 (m, 1H), 7.11-6.88 (m, 2H), 6.82-6.67 (m, 1H), 5.84-5.70 (m, 1H), 5.55-5.41 (m, 1H), 4.88-4.74 (m, 2H), 4.71-4.50 (m, 3H), 4.51-4.38 (m, 2H), 4.11 (d, J=17.2 Hz, 1H), 3.99-3.88 (m, 1H), 3.85-3.70 (m, 3H), 3.65-3.48 (m, 2H), 3.24-3.09 (m, 2H), 3.00-2.90 (m, 1H), 2.78-2.56 (m, 1H), 2.34-2.18 (m, 5H), 2.08-1.85 (m, 8H), 1.59-1.45 (m, 8H), 1.34-1.12 (m, 3H).

### Example 58: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3-methyl-3-(4-methyl-2-(2-morpholino-2-oxoethyl)-6-(phosphonooxy)phenyl)butanoate

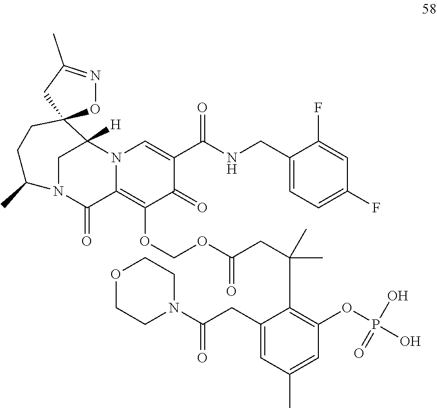 [SMILES: CC1=NO[C@@]2(CC[C@H](C)N3C[C@H]2n2cc(C(=O)NCc4ccc(F)cc4F)c(=O)c(OCOC(=O)CC(C)(C)c4c(CC(=O)N5CCOCC5)cc(C)cc4OP(=O)(O)O)c2C3=O)C1]

The title compound was made following the same method as Example 43, except in step 1 H-Asp(OBzl)-OBzl HCl was replaced with morpholine. MS (m/z) 914.7 [M+H]+. ¹H NMR (400 MHz, DMSO) δ 10.29 (t, J=5.9 Hz, 1H), 8.68 (s, 1H), 7.49-7.34 (m, 11H), 7.34-7.17 (m, 1H), 7.17-7.02 (m, 2H), 6.58-6.44 (m, 1H), 5.68 (d, J=6.2 Hz, 1H), 5.46 (d, J=6.2 Hz, 1H), 4.71-4.44 (m, 4H), 3.98-3.86 (m, 2H), 3.69-3.66 (m, 3H), 3.59-3.49 (m, 3H), 3.49-3.36 (m, 6H), 3.12-2.93 (m, 2H), 2.93-2.79 (m, 1H), 2.70-2.53 (m, 1H), 2.16 (s, 3H), 1.95 (s, 3H), 1.89-1.69 (m, 3H), 1.54-1.39 (m, 6H), 1.39-1.25 (m, 1H), 1.15 (d, J=6.6 Hz, 3H).

### Example 59: Preparation of N-((2-(((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)-N-(((phosphonooxy)methoxy)carbonyl)glycine (59)

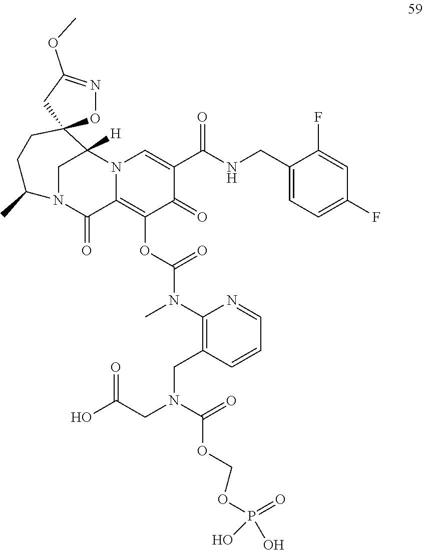 [SMILES: COC1=NO[C@@]2(CC[C@H](C)N3C[C@H]2n2cc(C(=O)NCc4ccc(F)cc4F)c(=O)c(OC(=O)N(C)c4ncccc4CN(CC(=O)O)C(=O)OCOP(=O)(O)O)c2C3=O)C1]

The title compound was synthesized following the same procedure as Steps 1-4 of Example 6, except starting from (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate C) instead of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B). MS (m/z) 877.7 [M+H]⁺. 1H NMR (400 MHz, MeOD) δ 8.88-8.62 (m, 1H), 8.53-8.37 (m, 1H), 8.22-7.91 (m, 1H), 7.60-7.36 (m, 2H), 7.03-6.85 (m, 2H), 5.73-5.42 (m, 2H), 5.20-4.99 (m, 1H), 4.86-4.48 (m, 5H), 4.43-4.02 (m, 1H), 3.96-3.64 (m, 5H), 3.64-3.43 (m, 1H), 3.42-3.35 (m, 7H), 3.23-3.07 (m, 1H), 2.82-2.56 (m, 1H), 2.17-1.80 (m, 3H), 1.79-1.43 (m, 1H), 1.34-1.20 (m, 3H).

### Example 60: Preparation of N-((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)-N-(((phosphonooxy)methoxy)carbonyl)glycyl-L-glutamic acid (60)

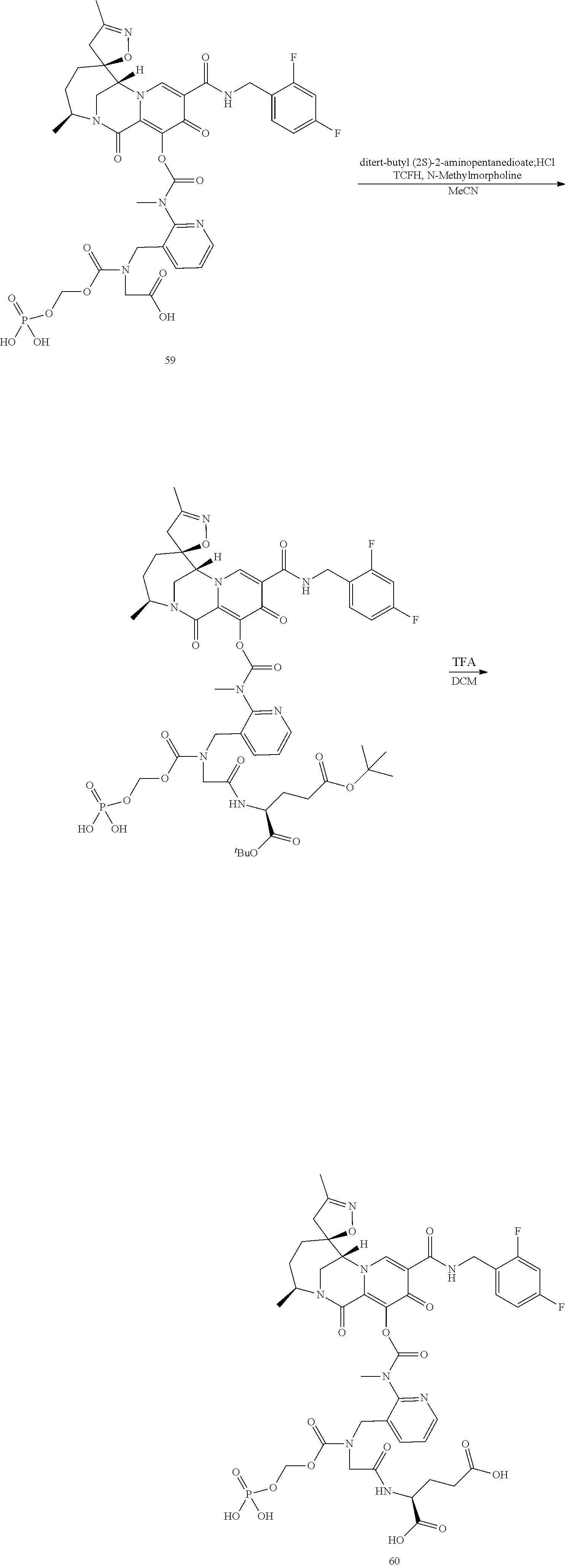

### Step 1: Preparation of di-tert-butyl N-((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)-N-(((phosphonooxy)methoxy)carbonyl)glycyl-L-glutamate

To a mixture of N-((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)-N-(((phosphonooxy)methoxy)carbonyl)glycine (79 mg, 0.088 mmol) and ditert-butyl (2S)-2-aminopentanedioate hydrochloride (Example 59, 39 mg, 0.133 mmol) in ACN (2 mL) at 0° C. was added N-Methylmorpholine (27 mg, 0.26 mmol) followed by TCFH (30 mg, 0.11 mmol). The mixture was stirred at 0° C. for 30 minutes and then stirred at room temperature for 30 minutes. Then the reaction mixture was concentrated and the resulting residue was purified by silica gel chromatography (0-100% EtOAc) to afford the title compound. MS (m/z) 1132.7 [M+H]⁺.

### Step 2: Preparation of N-((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)-N-(((phosphonooxyi)methoxy)carbonyl)glycyl-L-gilutamnic acid (60)

The title compound was made following the same method as Example 23 Step 3, except using di-tert-butyl N-((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)-N-(((phosphonooxy)methoxy)carbonyl)glycyl-L-glutamate instead of tert-butyl 2-(3-((di-tert-butoxyphosphoryl)oxy)-2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methylphenyl)acetate. MS (m/z) 1020.6 [M+H]⁺. 1H NMR (400 MHz, MeOD) δ 8.54 (s, 1H), 8.49-8.22 (m, 2H), 8.04-7.83 (m, 1H), 7.57-7.32 (m, 3H), 6.96 (s, 3H), 6.00-5.48 (m, 5H), 4.76-4.27 (m, 5H), 4.26-3.94 (m, 3H), 3.81 (s, 3H), 3.27-3.04 (m, 3H), 2.86-2.53 (m, 1H), 2.53-2.09 (m, 3H), 2.09-2.00 (m, 3H), 2.01-1.76 (m, 6H), 1.65-1.52 (m, 1H), 1.28 (d, J=6.6 Hz, 4H).

### Example 61: Preparation of (phosphonooxy)methyl ((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)(methyl)carbamate (61)

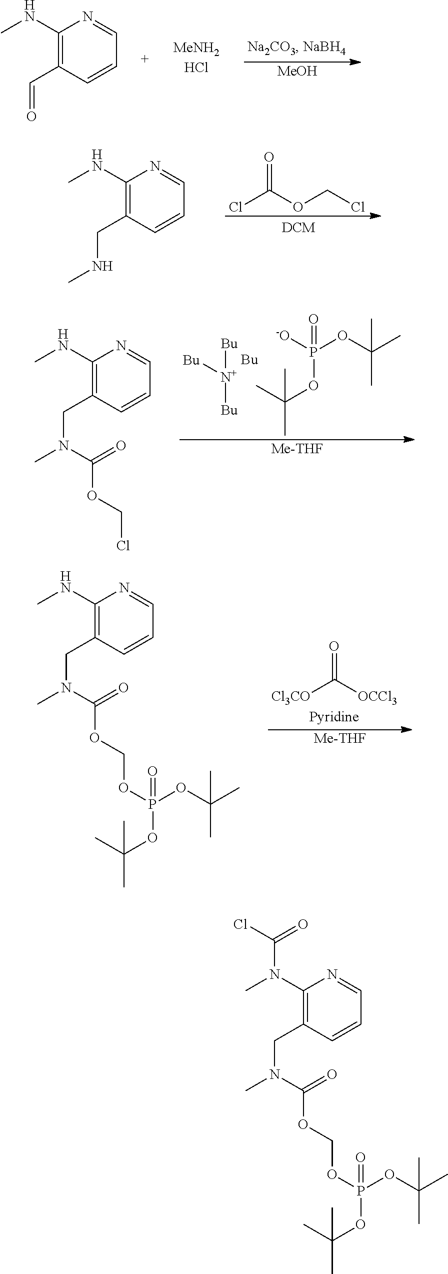

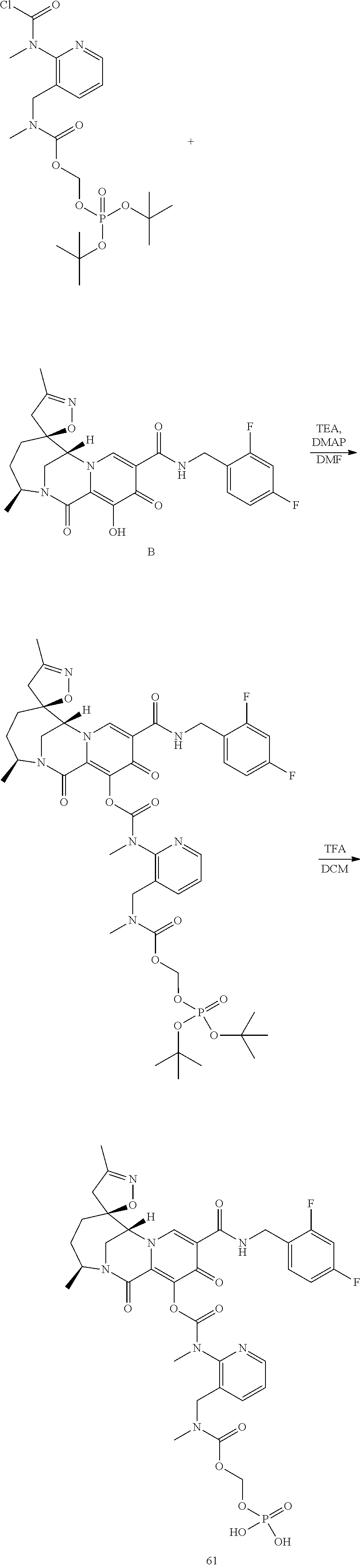

### Step 1: Preparation of N-methyl-3-((methylamino)methyl)pyridin-2-amine

Methylamine hydrochloride (15.6 g, 231 mmol) was mixed with MeOH (40 ml) at room temperature. K₂CO₃ (16.2 g, 118 mmol) was added in one portion. The reaction mixture was stirred at room temperature in a sealed reaction vessel for 30 min. Then to this reaction mixture was added a solution of 2-(methylamino)nicotinaldehyde (10.0 g, 73.4 mmol) in MeOH (5 mL). This reaction mixture was stirred at room temperature for 5 h and was then cooled down to 0° C. And NaBH₄ (2.83 g, 73.3 mmol) was added slowly. The resulting reaction mixture was then warmed up to room temperature and was stirred for 3 h. The final reaction mixture was then filtered through celite, concentrated to afford the title compound, concentrated, and used directly in the next step without purification. MS (m/z) 151.93 [M+H]+.

### Step 2: Preparation of chloromethyl methyl((2-(methylamino)pyridin-3-yl)methyl)carbamate

The crude product from step 1, containing N-methyl-3-((methylamino)methyl)pyridin-2-amine (10.8 g, 71.4 mmol) was mixed with DCM (200 ml) and the slurry was cooled down to −5° C. Then a solution of chloromethyl chloroformate (8.75 g, 67.9 mmol) in DCM (5 ml) was added slowly over 15 min. Let the reaction be stirred at the same temperature for 1 h. To the reaction mixture was added a saturated aqueous solution of NaHCO₃ (50 mL) at 0° C. The resulting reaction mixture was then stirred at the same temp for 10 min. EtOAc (500 ml) was added. Organic layer was separated and was washed with brine (50 ml). The organic phase was dried over Na₂SO₄ and was filtered. The filtrate was concentrated to dryness to afford the title compound. MS (m/z) 244.10 [M+H]+.

### Step 3: Preparation of ((di-tert-butoxyphosphoryl)oxy)methyl methyl((2-(methylamino)pyridin-3-yl)methyl)carbamate

Chloromethyl methyl((2-(methylamino)pyridin-3-yl)methyl)carbamate (17.4 g, 71.4 mmol) and tetrabutylammonium di-tert-butyl phosphate (67 g, 148 mmol) were mixed with Me-THF (200 ml). The slurry was heated up to 78° C., and kept at that temp with stirring for 2 h. The reaction mixture was cooled down to room temperature and was partitioned between water (200 ml) and EtOAc (200 ml). The organic phase was separated and washed with brine (50 ml). The organic extraction was concentrated. The residue was purified on silica gel with (0-100% EtOAc in Hexanes) to afford the title compound. MS (m/z) 417.97 [M+H]+.

### Step 4: Preparation of ((di-tert-butoxyphosphoryl)oxy)methyl ((2-((chlorocarbonyl)(methyl)amino)pyridin-3-yl)methyl)(methyl)carbamate

((Di-tert-butoxyphosphoryl)oxy)methyl methyl((2-(methylamino)pyridin-3-yl)methyl)carbamate (4 g, 9.58 mmol) was dissolved in Me-THF (80 ml) at room temperature. Under argon balloon, the solution was cooled down to 0° C. Then pyridine (1.14 g, 14.4 mmol) was added with stirring. Ten minutes later, triphosgene (2.61 g, 8.62 mmol) was added in one portion as solid. The reaction mixture was stirred at room temperature overnight and was filtered. The filter cake was washed with Me-THF (10 ml) and EtOAc (10 ml). The filtrate was treated with HCl (IN) (20 ml) and brine (20 ml) and then was dried over Na₂SO₄. The resulting solution was concentrated to afford the title compound. MS (m/z) 479.49 [M+H]+.

### Step 5: Preparation of ((di-tert-butoxyphosphoryl)oxy)methyl ((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl) (methyl)carbamate

((Di-tert-butoxyphosphoryl)oxy)methyl ((2-((chlorocarbonyl)(methyl)amino)pyridin-3-yl)methyl)(methyl)carbamate (4.6 g, 9.59 mmol) and (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (intermediate B, 3 g, 6.17 mmol) were dissolved in DMF (30 ml) at room temperature. Triethylamine (1.87 g, 18.5 mmol) and DMAP (0.45 g, 3.7 mmol) were added sequentially. The reaction mixture was stirred at room temperature for 2 h. Reaction mixture was then diluted with EtOAc (30 ml) and was treated with saturated aqueous solution of NH₄Cl (10 ml). The aqueous layer was extracted with EtOAc, washed with brine and concentrated. The residue was purified by silica gel chromatography (0-100% EtOAc) to afford the title compound. MS (m/z) 929.60 [M+H]+.

### Step 6: Preparation of (phosphonooxy)methyl ((2-(((((3′S,5S,7′R)-10′-((2,4-difluorobenzyl)carbamoyl)-3,3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)(methyl)carbamate (61)

((Di-tert-butoxyphosphoryl)oxy)methyl ((2-(((((3′S,5S,7′R)-10′-((2,4-difluorobenzyl)carbamoyl)-3,3′-dimethyl-1′,11′-dioxo-646′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)(methyl)carbamate (3.7 g, 3.98 mmol) was dissolved in DCM (160 ml) at room temperature. The solution was cooled down to 0° C. under argon atmosphere. Trifluoroacetic acid (25.8 g, 228 mmol) was added dropwise over 25 min. Reaction was stirred at 0° C. for 70 min. Then TFA and DCM was removed under reduced pressure. The residue was triturated with hexane to afford the title compound. MS (m/z): 817.93 [M+H]+. ¹H NMR (400 MHz, DMSO) δ 10.18 (s, 1H), 8.77-8.73 (m, 1H), 8.47-8.43 (m, 1H), 7.67-7.63 (m, 1H), 7.43-7.36 (m, 2H), 7.28-7.18 (m, 1H), 7.12-7.02 (m, 1H), 6.88 (s, 1H), 5.56-5.48 (m, 2H), 4.75-4.53 (m, 5H), 3.93-3.59 (m, 3H), 3.48-3.32 (m, 3H), 3.29-3.10 (m, 2H), 3.10-2.72 (m, 3H), 2.72-2.59 (m, 1H), 2.19 (s, 1H), 1.94 (s, 4H), 1.83-1.68 (m, 1H), 1.60-1.48 (m, 1H), 1.17-1.13 (m, 3H).

### Example 62: Preparation of (phosphonooxy)methyl ((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)(methyl)carbamate (62)

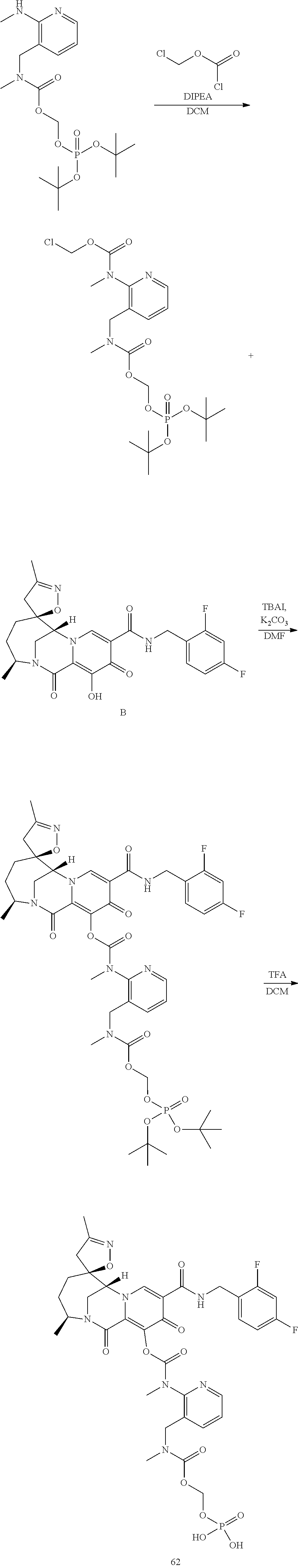

### Step 1: Preparation of ((di-tert-butoxyphosphoryl)oxy)methyl (2-(((chloromethoxy)carbonyl)(methyl) amino)pyridin-3-yl)methyl)(methyl)carbamate

((Di-tert-butoxyphosphoryl)oxy)methyl methyl((2-(methylamino)pyridin-3-yl)methyl)carbamate (100 mg, 0.24 mmol) was dissolved in DCM (10 mL) at 0° C. DIPEA (49.4 mg, 0.383 mmol) was added. And then a solution of chloromethyl chloroformate (15 mg, 0.12 mmol) in DCM (0.5 ml) was added. Reaction mixture was stirred at room temperature for 1 h. Then the second portion of DIEA (18.5 mg, 0.144 mmol) and a solution of chloromethyl chloroformate (15 mg, 0.12 mmol) in DCM (0.5 ml) were added sequentially. The reaction mixture was concentrated for the removal of DCM. The residue was taken up in EtOAc (10 ml) and was treated with saturated ammonium chloride (10 ml), concentrated and the residue was purified on silica gel column with (0-100% EtOAc) to afford the title compound. MS (m/z) 509.87 [M+H]+.

### Step 2: Preparation of ((di-tert-butoxyphosphoryl)oxy)methyl ((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)(methyl)carbamate

((Di-tert-butoxyphosphoryl)oxy)methyl ((2-(((chloromethoxy)carbonyl)(methyl) amino)pyridin-3-yl)methyl)(methyl)carbamate (58 mg, 0.114 mg) and (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 40 mg, 0.0822 mmol) were mixed with DMF (2 mL) at room temperature. Potassium carbonate (32 mg, 0.232 mmol) and tetrabutylammonium iodide (30.4 mg, 0.0822 mmol) were added sequentially and the reaction mixture was at 60° C. for 4 hr. Then the reaction mixture was stirred at room temperature overnight. The reaction mixture was then diluted with EtOAc (20 mL), washed with water (10 mL) and brine (10 mL), dried, concentrated and purified by silica gel chromatography (0-100% EtOAc followed by 10% MeOH/EtOAc) to afford the title compound. MS (m/z) 960.10 [M+H]+.

### Step 3: Preparation of (phosphonooxy)methyl ((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)(methyl)carbamate (62)

((Di-tert-butoxyphosphoryl)oxy)methyl ((2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)pyridin-3-yl)methyl)(methyl)carbamate (62 mg, 0.0646 mmol) was dissolved in DCM (3 mL) at room temperature. The solution was cooled down to 0° C. Then trifluoroacetic acid (0.3 mL, 3.92 mmol) was added dropwise. The reaction mixture was then stirred at the same temperature for 90 min. The solvent and toluene by product were removed under high vacuum to provide the title compound. MS (m/z) 847.90 [M+H]+. ¹H NMR (400 MHz, DMSO) δ 10.48-10.15 (m, 1H), 8.88-8.54 (m, 1H), 8.54-8.28 (m, 1H), 7.81-7.58 (m, 1H), 7.48-7.33 (m, 2H), 7.30-7.19 (m, 1H), 7.12-7.03 (m, 1H), 5.92-5.66 (m, 2H), 5.57-5.29 (m, 2H), 4.78-4.58 (m, 1H), 4.62-4.49 (m, 2H), 4.46-4.36 (m, 2H), 3.80-3.66 (m, 2H), 3.66-3.58 (m, 1H), 3.45-3.26 (m, 1H), 3.26-3.07 (m, 3H), 3.07-2.96 (m, 1H), 2.96-2.55 (m, 4H), 1.95 (s, 3H), 1.89-1.67 (m, 4H), 1.61-1.44 (m, 1H), 1.18 (d, J=6.7 Hz, 3H).

### Example 63: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (3R)-3-((phosphonooxy)methyl)morpholine-4-carboxylate (63)

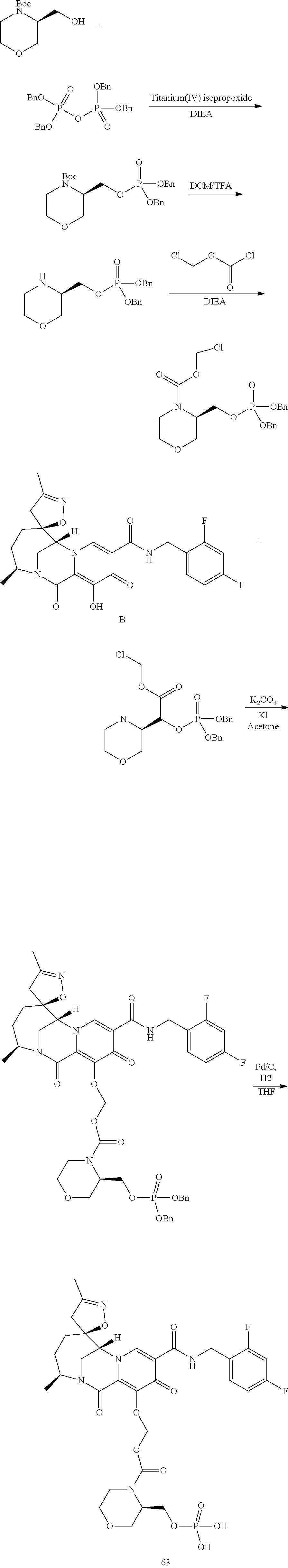

### Step 1: Synthesis of tert-butyl (R)-3-((bis(benzyloxy)phosphoryl)oxy)methylmorpholine-4-carboxylate

To a mixture of tert-butyl (S)-3-(hydroxymethyl)morpholine-4-carboxylate (332 mg, 1.53 mmol) in DCM (1.5 mL) at room temperature was added DIEA (494 mg, 3.82 mmol) followed by tetrabenzyl diphosphate (741 mg, 1.38 mmol) and Titanium(IV) isopropoxide (73.8 mg, 0.26 mmol). The resulting mixture was stirred for 1 hr. A mixture of Magnesium sulfate and silica gel (4:1) was added to the reaction and stirred for 10 minutes. The mixture was filtered and filter cake was rinsed with EtOAc/Hexane (4:3), concentrated and purified by normal phase silica chromatography to provide title compound (0-100% EtOAc). MS (m/z) 477.828 [M+H]+

### Step 2: Synthesis of (R)-dibenzyl (morpholin-3-ylmethyl) phosphate

tert-butyl (R)-3-(((bis(benzyloxy)phosphoryl)oxy)methyl)morpholine-4-carboxylate (230 mg, 0.482 mmol) was dissolved in DCM (2.0 mL) and cooled to 0° C. To this cold solution was added a mixture of TFA (0.75 mL) in DCM (1.0 mL) slowly and stirred for 30 minutes at 0° C. before it was removed from cooling bath and stirred for another 30 minutes at room temperature. The reaction was then concentrated, residue redissolved in DMF, filtered and purified by reverse phase prep HPLC (5-100% MeCN/H₂O w/ 0.1% TFA) to give title compound as TFA salt. MS (m/z) 378.81 [M+H]+

### Step 3: Synthesis of chloromethyl (R)-3-(((bis(benzyloxy)phosphoryl)oxy)methyl)morpholine-4-carboxylate

(R)-dibenzyl (morpholin-3-ylmethyl) phosphate TFA salt (95 mg, 0.193 mmol) was dissolved in DCM and cooled to 0° C., DIEA (75.0 mg, 0.580 mmol) was added followed by chloromethyl carbonochloridate (29.9 mg, 0.232 mmol). After 30 minutes, the reaction was diluted with DCM, washed successively with water, IN HCl, brine, dried over sodium sulfate, filtered and concentrated to give title compound, used directly in next step. MS (m/z) 469.778 [M]+

### Step 4: Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (3R)-3-(((bis(benzyloxy)phosphoryl)oxy)methyl)morpholine-4-carboxylate

To a mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 70.0 mg, 0.144 mmol) and chloromethyl (R)-3-(((bis(benzyloxy)phosphoryl)oxy)methyl)morpholine-4-carboxylate (91.3 mg, 0.194 mmol) in acetone (5.0 mL) was added potassium carbonate (39.8 mg, 0.288 mmol) and potassium iodide (32.2 mg, 0.194 mmol). The resulting mixture was stirred for 18 hours before it was diluted with EtOAc, washed with water, brine, dried over sodium sulfate, filtered and concentrated and used directly in next step. MS (m/z) 919.939 [M+H]+

### Step 5: Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl oxy)methyl (3R)-3-((phosphonooxy)methyl)morpholine-4-carboxylate (63)

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (3R)-3-(((bis(benzyloxy)phosphoryl)oxy)methyl)morpholine-4-carboxylate (50 mg, 0.054 mmol) was dissolved in THF (5 mL) at room temperature. To this stirred solution was added 10% Pd/C (5 mg). The mixture was degassed and flushed with nitrogen three times and then degassed and flushed with hydrogen three times before it was hydrogenated under hydrogen balloon for 3 hrs. the reaction was then degassed and flushed with nitrogen, filtered through a pad of Celite®, concentrated, the residue was redissolved in DMF, filtered and purified by reverse phase prep HPLC (5-100% MeCN/H₂O w/ 0.1% TFA) to give title compound. MS (m/z) 739.909 [M+H]+. ¹H NMR (400 MHz, MeOD) δ 8.57 (s, 1H), 7.59-7.36 (m, 1H), 7.10-6.81 (m, 2H), 6.02-5.85 (m, 1H), 5.85-5.60 (m, 1H), 4.88-4.80 (m, 2H), 4.79-4.56 (m, 2H), 4.56-4.33 (m, 2H), 4.28-4.15 (m, 3H), 4.15-4.06 (m, 1H), 4.04-3.88 (m, 1H), 3.88-3.71 (m, 4H), 3.65-3.35 (m, 2H), 3.22-3.03 (m, 2H), 2.70 (d, J=17.9 Hz, 1H), 2.06 (s, 3H), 2.02-1.85 (m, 3H), 1.53 (d, J=13.5 Hz, 1H), 1.27 (d, J=6.7 Hz, 3H).

### Example 64: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (3S)-3-((phosphonooxy)methyl)morpholine-4-carboxylate (64)

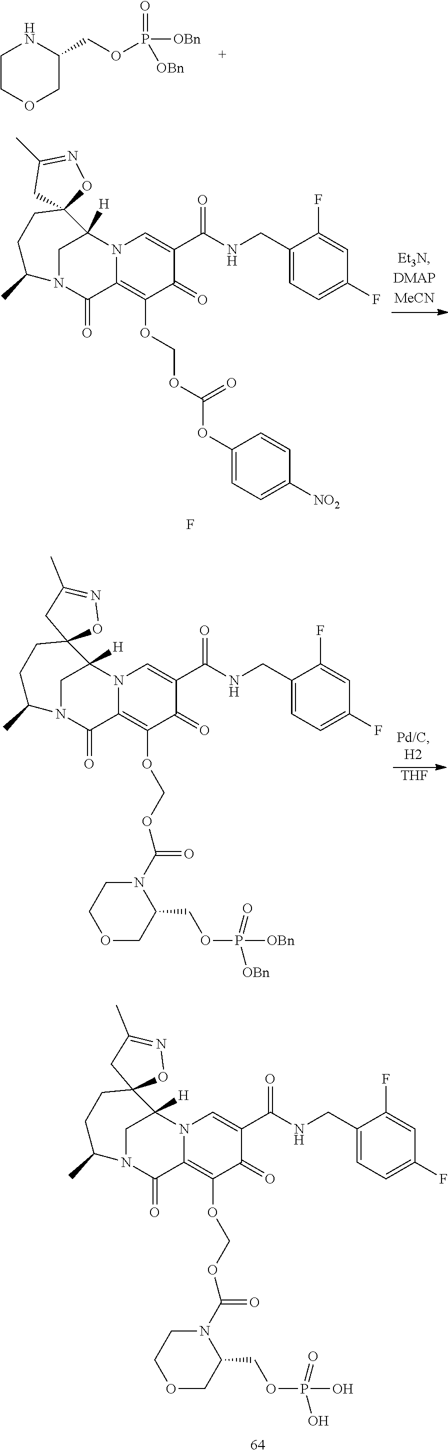

### Step 1: Synthesis of (S)-dibenzyl (morpholin-3-ylmethyl) phosphate

The title compound was prepared in similar manner as (R)-dibenzyl (morpholin-3-ylmethyl) phosphate, except (R)-3-(hydroxymethyl)morpholine-4-carboxylate was used in step 1. MS (m/z) 378.05 [M+H]+

### Step 2: Synthesis of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (3S)-3-(((bis(benzyloxy)phosphoryl)oxy)methyl)morpholine-4-carboxylate

The title compound was prepared following the same method as in Example 37 step 2, except (S)-dibenzyl (morpholin-3-ylmethyl) phosphate was used in place of di-tert-butyl (2-((methylamino)methyl)phenyl) phosphate and triethylamine was used in place of DIEA. MS (m/z) 919.81 [M+H]+

### Step 3: Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (3S)-3-(phosphonooxy)methyl)morpholine-4-carboxylate (64)

The title compound was prepared in a similar manner as Example 63. MS (m/z) 739.92 [M+H]+. ¹H NMR (400 MHz, MeOD) δ 8.68 (s, 1H), 7.52-7.30 (m, 1H), 7.08-6.83 (m, 2H), 4.80-4.41 (m, 6H), 4.36-4.02 (m, 5H), 3.98-3.58 (m, 8H), 3.22-3.10 (m, 1H), 2.70 (d, J=17.9 Hz, 1H), 2.07 (s, 3H), 2.01-1.87 (m, 2H), 1.70-1.48 (m, 1H), 1.27 (d, J=6.7 Hz, 3H).

### Example 65: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3S)-3-((phosphonooxy)methyl)morpholine-4-carboxylate (65)

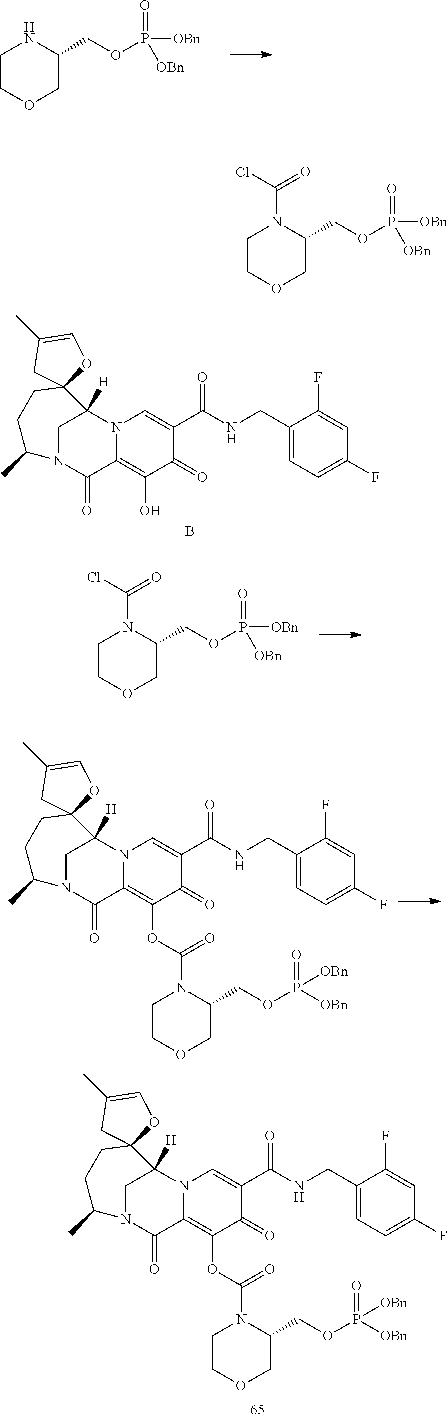

### Step 1: Synthesis of (S)-dibenzyl ((4-(chlorocarbonyl)morpholin-3-yl)methyl) phosphate

The title compound was prepared in a similar manner as Example 33 step 5, except replaced benzyl N-((((bis(benzyloxy)phosphoryl)oxy)methoxy)carbonyl)-N-((2-((chlorocarbonyl)(methyl)amino)pyridin-3-yl)methyl)glycinate with (R)-3-(hydroxymethyl)morpholine-4-carboxylate. MS (m/z) 439.86 [M+H]+

### Step 2: Synthesis of (3′S,5S,7′R)-10′-((2,4-difluorobenzyl)carbamoyl)-3,3′-dimethyl-1′,11′-dioxo-1′,4′,5′,11′-tetrahydro-3′H,4H7′H-spiro[isoxazole-5,6′-[2,7]methanopyrido[1,2-a][1,4]diazonin]-12′-yl (3S)-3-(((bis(benzyloxy)phosphoryl)oxy)methyl)morpholine-4-carboxylate

The title compound was prepared in a similar manner as Example 33 step 6, except (S)-dibenzyl ((4-(chlorocarbonyl)morpholin-3-yl)methyl) phosphate was used instead of benzyl N-((((bis(benzyloxy)phosphoryl)oxy)methoxy)carbonyl)-N-((2-((chlorocarbonyl)(methyl)amino)pyridin-3-yl)methyl)glycinate. MS (m/z) 889.89 [M+H]+.

### Step 3: Synthesis of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3S)-3-((phosphonooxy)methyl)morpholine-4-carboxylate (65)

The title compound was prepared in the same manner as Example 33 step 7, except (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3S)-3-(((bis(benzyloxy)phosphoryl)oxy)methyl)morpholine-4-carboxylate was used. MS (m/z) 709.86 [M+H]+. 1H NMR (400 MHz, MeOD) δ 8.68 (s, 1H), 7.54-7.28 (m, 1H), 7.04-6.91 (m, 2H), 4.86-3.54 (m, 19H), 3.23-3.06 (m, 1H), 2.70 (d, J=17.9 Hz, 1H), 2.13-2.01 (m, 3H), 2.01-1.79 (m, 2H), 1.73-1.48 (m, 1H), 1.36-1.20 (m, 3H).

### Example 66: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2S)-2-((phosphonooxy)methyl)pyrrolidine-1-carboxylate (66)

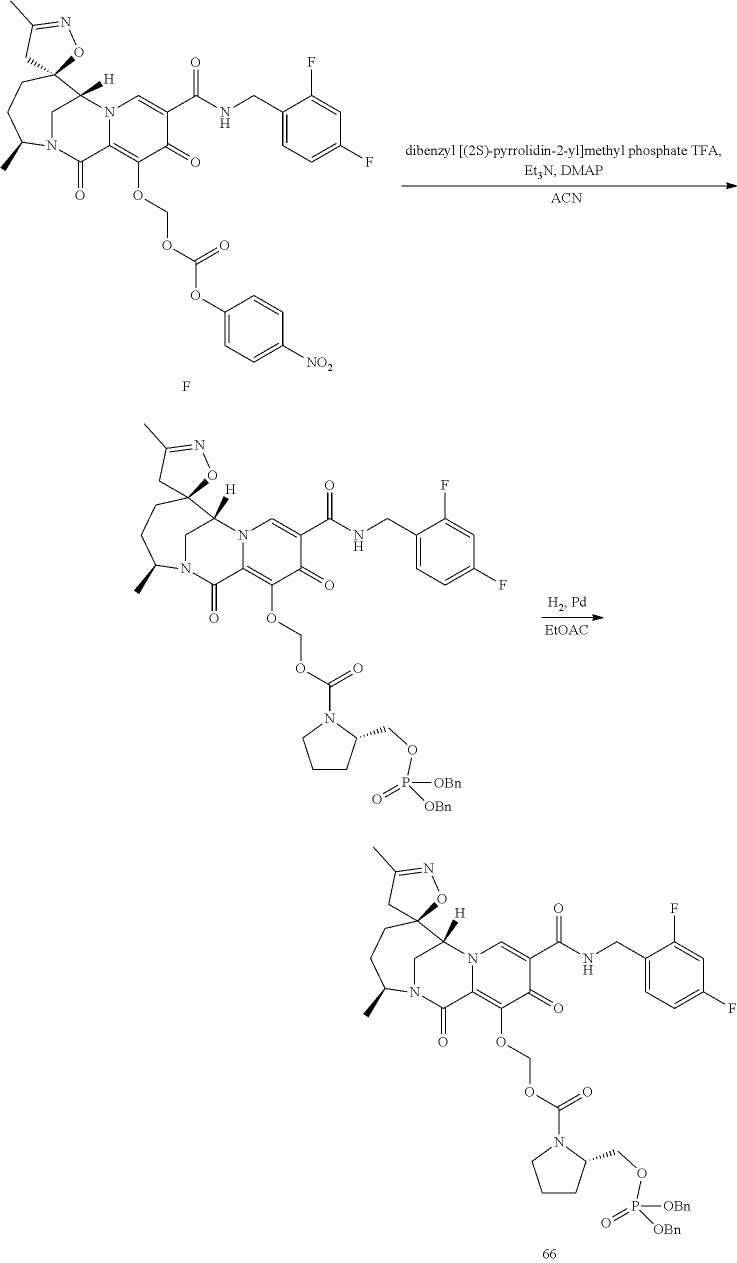

### Step 1: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2S)-2-(((bis(benzyloxy)phosphoryl)oxy)methyl)pyrrolidine-1-carboxylate

To a mixture of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (intermediate F, 100 mg, 0.15 mmol) and dibenzyl [(2S)-pyrrolidin-2-yl]methyl phosphate TFA (135 mg, 0.29 mmol) in ACN (2 mL) at RT was added Et₃N (59 mg, 0.58 mmol) followed by DMAP (2 mg, 0.014 mmol). The mixture was stirred at room temperature for 30 minutes. Then it was concentrated and the resulting residue was purified by silica gel chromatography (0-100% EtOAc) to afford the title compound. MS (m/z) 903.7 [M+H]⁺.

### Step 2: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2S)-2-((phosphonooxy)methyl)pyrrolidine-1-carboxylate (66)

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2S)-2-(((bis(benzyloxy)phosphoryl)oxy)methyl)pyrrolidine-1-carboxylate (108 mg, 0.095 mmol) was dissolved in EtAOc (2 mL) and to the solution was added Pd (10% Pd on carbon, 20 mg), the reaction flask was then vacuumed and charged with H₂using hydrogen balloon. The reaction mixture was stirred at RT for 3 h and then filtered. The filtrate was then evaporated and purified by reverse phase preparative HPLC (5-100% MeCN/H₂O w/ 0.1% TFA) to afford the title compound. MS (m/z) 723.8 [M+H]⁺. ¹H NMR (400 MHz, MeOD) δ 8.57 (s, 1H), 7.52-7.38 (m, 1H), 7.08-6.87 (m, 2H), 6.04-5.79 (m, 11H), 5.79-5.56 (m, 1H), 4.94-4.90 (m, 1H), 4.86-4.75 (m, 1H), 4.74-4.57 (m, 3H), 4.47 (s, 1H), 4.15-3.99 (m, 1H), 3.99-3.69 (m, 4H), 3.54-3.40 (m, 1H), 3.40-3.34 (m, OH), 3.25-3.06 (m, 1H), 2.79-2.63 (m, 1H), 2.11-1.81 (m, 8H), 1.66-1.49 (m, 1H), 1.26 (d, J=6.7 Hz, 3H).

### Example 67: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2S)-2-((phosphonooxy)methyl)azetidine-1-carboxylate (67)

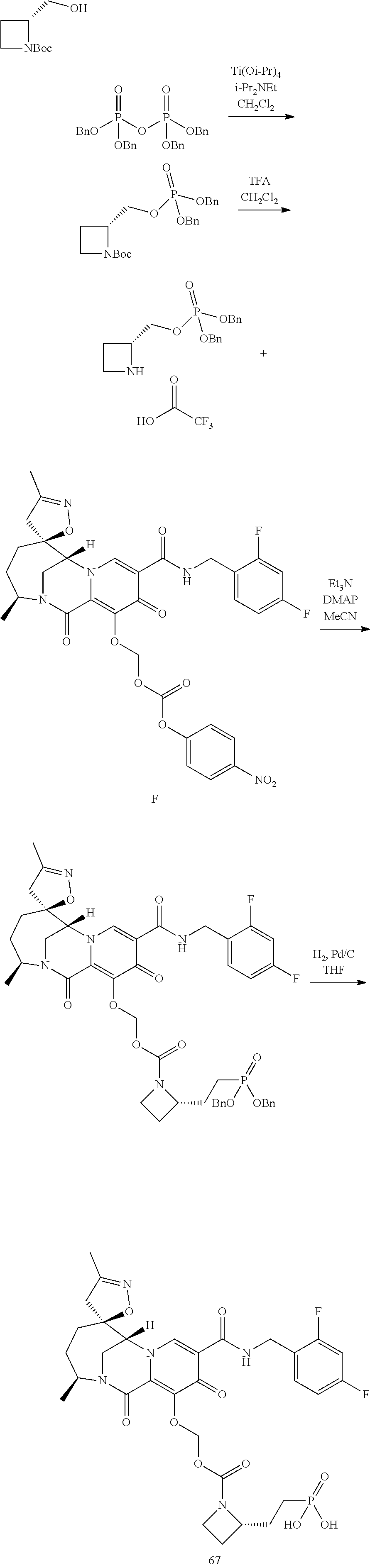

### Step 1: Preparation of tert-butyl (R)-2-(((bis(benzyloxy)phosphoryl)oxy)methyl)azetidine-1-carboxylate

To a mixture of tert-butyl (R)-2-(hydroxymethyl)azetidine-1-carboxylate (0.389 g, 2.08 mmol) in CH₂C12 (1 mL) was added DIPEA (0.415 mL, 2.31 mmol) followed by tetrabenzylpyrophosphate (0.500 g, 0.929 mmol) and Ti(iPrO)4 (0.047 mL, 0.158 mmol). The mixture was stirred at rt overnight. MgSO4 and silica gel (4:1) were added to reaction and stirred for 10 min. The mixture was filtered and the filter cake was washed with EtOAc/hexanes (4:3). The filtrate was concentrated and purified by reverse phase preparative HPLC (10-100% MeCN/water) and lyophilized to afford tert-butyl (R)-2-(((bis(benzyloxy)phosphoryl)oxy)methyl)azetidine-1-carboxylate. MS (m/z) 447.72 [M+H]+.

### Step 2: Preparation of (R)-azetidin-2-ylmethyl dibenzyl phosphate; trifluoroacetic acid salt

To a flask containing tert-butyl (R)-2-(((bis(benzyloxy)phosphoryl)oxy)methyl)azetidine-1-carboxylate (0.190 g, 0.425 mmol) at 0° C. was added a 0° C. solution of TFA (0.162 mL, 2.12 mmol) in CH₂C₁₋₂ (4.2 mL). The reaction mixture was allowed to warm to rt, stir for 3 d, and concentrated to afford (R)-azetidin-2-ylmethyl dibenzyl phosphate; trifluoroacetic acid salt. MS (m/z) 348.08 [M+H]⁺.

### Step 3: Preparation of (((3′S,5S,7′R)-10′-((2,4-difluorobenzyl)carbamoyl)-3,3′-dimethyl-1′,11′-dioxo-1′,4′,5′,11′-terrahydro-3′H,4H,7′H-spiro[isoxazole-5,6′-[2,7]methanopyrido[1,2-a][1,4]diazonin]-12′-yl)oxy)methyl (2S)-2-(((bis(benzyloxy)phosphoryl)oxy)methyl)azetidine-1-carboxylate

To a solution of Intermediate F (0.120 g, 0.176 mmol) in MeCN (4 mL) was added Et₃N (0.147 mL, 1.06 mmol) and DMAP (0.002 g, 0.018 mmol), followed by (R)-azetidin-2-ylmethyl dibenzyl phosphate; trifluoroacetic acid salt (0.244 mL, 0.528 mmol). The reaction mixture was left to stir at rt overnight and was concentrated. The residue was purified by silica gel column chromatography (0-100% EtOAc/hexanes, then 0-20% MeOH/DCM) to afford (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2S)-2-(((bis(benzyloxy)phosphoryl)oxy)methyl)azetidine-1-carboxylate. MS (m/z) 889.92 [M+H]⁺.

### Step 4: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2S)-2-((phosphonooxy)methyl)azetidine-1-carboxylate

To a solution of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2S)-2-(((bis(benzyloxy)phosphoryl)oxy)methyl)azetidine-1-carboxylate (0.152 g, 0.171 mmol) in THF (17 mL) was added 10 wt % Pd/C (0.018 g, 0.017 mmol). The mixture was evacuated and backfilled with H₂ (g) (2×) then sparged with H₂ for 5 min. The reaction mixture was left to stir overnight, filtered through Celite, and concentrated. The residue was purified by reverse phase preparative HPLC (10-100% MeCN/water w/ 0.1% TFA) to afford (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2S)-2-((phosphonooxy)methyl)azetidine-1-carboxylate. MS (m z) 709.93 [M+H]⁺. 1H NMR (400 MHz, DMSO-d6) δ 10.31 (t, J=6.0 Hz, 1H), 8.68 (s, 1H), 7.43 (td, J=8.7, 6.6 Hz, 1H), 7.25 (ddd, J=10.5, 9.3, 2.6 Hz, 1H), 7.08 (ddd, J=10.5, 7.9, 2.4 Hz, 1H), 5.91-5.35 (m, 2H), 4.77-4.60 (m, 2H), 4.60-4.48 (m, 2H), 4.30 (s, 1H), 3.95 (s, 2H), 3.77-3.56 (m, 3H), 3.00 (d, J=17.5 Hz, 1H), 2.64 (d, J=17.7 Hz, 1H), 2.26-2.04 (m, 2H), 1.95 (s, 3H), 1.90-1.68 (m, 3H), 1.32 (dd, J=15.5, 11.0 Hz, 1H), 1.18 (d, J=6.7 Hz, 3H).

### Example 68: Preparation of phosphonooxymethyl (2S)-2-[[[(1R,10S,13S)-4-[(2,4-difluorophenyl)methylcarbamoyl]-3′,10-dimethyl-5,8-dioxo-spiro[2,9-diazatricyclo[7.4.1.02,7]tetradeca-3,6-diene-13,5′-4H-isoxazole]-6-yl]oxycarbonyl-methylamino]methyl]pyrrolidine-1-carboxylate (68)

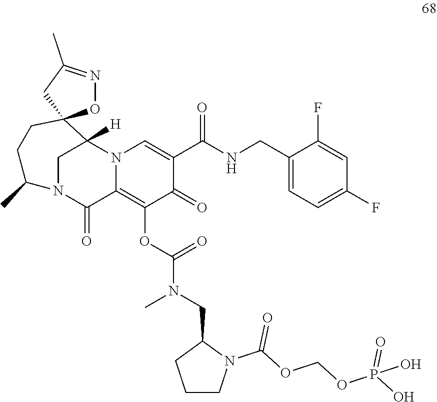 [SMILES: CC1=NO[C@@]2(CC[C@H](C)N3C[C@H]2n2cc(C(=O)NCc4ccc(F)cc4F)c(=O)c(OC(=O)N(C)C[C@@H]4CCCN4C(=O)OCOP(=O)(O)O)c2C3=O)C1]

Phosphonooxymethyl (2S)-2-[[[(1R,10S,13S)-4-[(2,4-difluorophenyl)methylcarbamoyl]-3′,10-dimethyl-5,8-dioxo-spiro[2,9-diazatricyclo[7.4.1.02,7]tetradeca-3,6-diene-13,5′-4H-isoxazole]-6-yl]oxycarbonyl-methylamino]methyl]pyrrolidine-1-carboxylate was made following the same method as KCl, except using tert-butyl N-methyl-N-[[(2S)-pyrrolidin-2-yl]methyl]carbamate instead of tert-butyl (3R)-3-[[(2-benzyloxy-2-oxo-ethyl)amino]methyl]morpholine-4-carboxylate. MS (m/z) 781.1 [M+H]. 1H NMR (400 MHz, Methanol-d4) δ 8.65 (d, J=5.7 Hz, 1H), 7.46 (td, J=8.5, 6.4 Hz, 1H), 7.09-6.85 (m, 2H), 5.79-5.48 (m, 2H), 4.82-4.47 (m, 4H), 4.38-4.08 (m, 1H), 3.94-3.38 (m, 7H), 3.30-3.00 (m, 5H), 2.70 (d, J=17.9 Hz, 1H), 2.13-1.83 (m, 9H), 1.60 (s, 1H), 1.26 (d, J=6.7 Hz, 3H).

### Example 69: Preparation of 2-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl)phenyl dihydrogen phosphate (69)

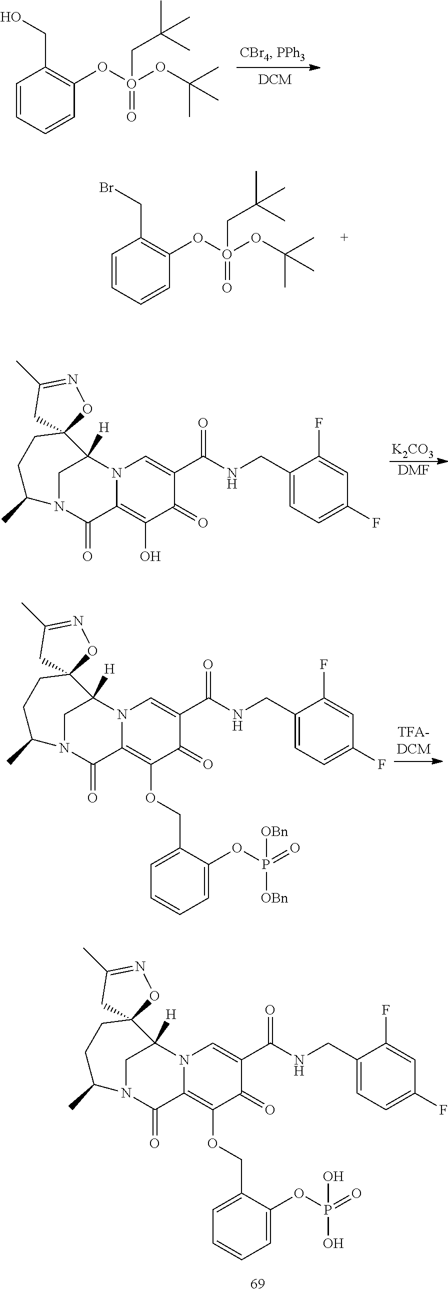

### Step 1: Preparation of 2-(bromomethyl)phenyl di-tert-butyl phosphate

To a solution of ditert-butyl [2-(hydroxymethyl)phenyl]phosphate (518 mg, 1.64 mmol), PPh₃ (472 mg, 1.8 mmol) in 10 mL dry DCM, the carbon tetrabromide (597 mg, 1.8 mmol) was added with several portion at 0° C. The mixture was stirred for overnight at room temperature. After that, the petroleum ether (100 mL) was added to the mixture solution, and the solid was filtered through a short the celite. The solvent was removed by vacuum, and the residue was purified by column chromatography (ethyl acetate in hexane from 0% to 40%) to provide 2-(bromomethyl)phenyl di-tert-butyl phosphate. 1H NMR (400 MHz, Chloroform-d) δ 7.44 (ddt, J=10.7, 7.6, 1.3 Hz, 2H), 7.33-7.29 (m, 1H), 7.17-7.09 (m, 1H), 4.61 (s, 2H), 1.55 (d, J=0.6 Hz, 18H).

### Steps 2 and 3: Preparation of 2-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl)phenyl dihydrogen phosphate (69)

2-((((3′S,5S,7R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl)phenyl dihydrogen phosphate was synthesized following the same procedure as Example 71, starting from 2-(bromomethyl)phenyl di-tert-butyl phosphate and (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide. MS (m/z) 673.8 [M+H]+. ¹H NMR (4(0) MHz, DMSO-d6) δ 10.36 (d, J=6.9 Hz, 1H), 8.70 (s, 1H), 7.70 (d, J=7.6 Hz, 1H), 7.42 (d, J=8.7 Hz, 1H), 7.31-7.17 (m, 4H), 7.07 (s, 1H), 5.33 (d, J=12.6 Hz, 1H), 5.21 (d, J=12.8 Hz, 1H), 4.63 (s, 2H), 4.55 (d, J=8.8 Hz, 2H), 3.73 (t, J=16.6 Hz, 2H), 3.04 (d, J=17.5 Hz, 1H), 2.67 (d, J=17.4 Hz, 1H), 1.95 (s, 3H), 1.76 (d, J=24.9 Hz, 3H), 1.35 (s, 1H), 1.15 (d, J=6.5 Hz, 3H).

### Intermediate K: Preparation ditert-butyl [4-(hydroxymethyl)-3-pyridyl] phosphate

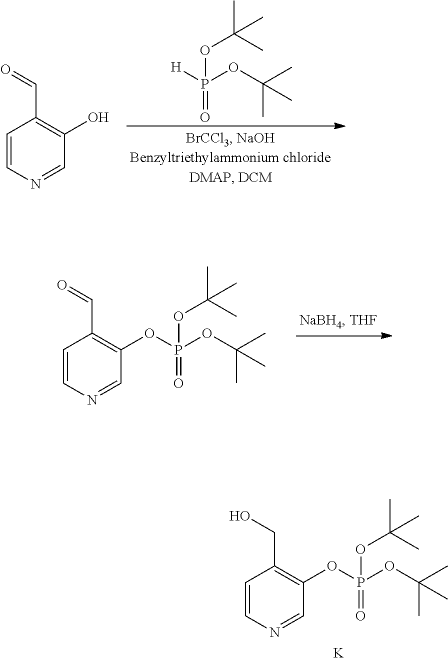

### Step 1: Synthesis of di-tert-butyl (4-formylpyridin-3-yl) phosphate

Benzyltriethylammonium chloride (139 mg, 0.61 mmol), DCM (10 mL), and bromotrichloromethane (1.127 g, 5.69 mmol), were added to a solution of sodium hydroxide (1.624 g, 40.6 mmol) in water (10 mL). To this biphasic mixture, vigorously stirred at 0° C., was added a solution of 2-tert-butoxyphosphonoyloxy-2-methyl-propane (1.025 g, 5.28 mmol) in DCM (10 mL) dropwise over 5 min. The reaction mixture was then allowed to warm to room temperature w % bile stirring vigorously for 2 h. at which point, a solution of 3-hydroxypyridine-4-carbaldehyde (0.5 g, 4.06 mmol) in DCM (10 mL) and DMAP (49.6 mg, 0.406 mmol) were added. The mixture was stirred for 1 h. The organic layer was then separated and washed with 10% aqueous citric acid solution and dried over anhydrous MgSO₄. After removing the solvent, the residue was concentrated to provide di-tert-butyl (4-formylpyridin-3-yl) phosphate. MS (m/z) 315.8 [M+H]⁺.

### Step 2: ditert-butyl [4-(hydroxymethyl)-3-pyridyl] phosphate (Intermediate K)

di-tert-butyl (4-formylpyridin-3-yl) phosphate (0.85 g, 2.68 mmol) was dissolved in THF (10 mL) and cooled to 0° C. followed by addition of a solution of NaBH₄ (154 mg, 4.06 mmol) in water (3 mL). The resulting reaction mixture was stirred for 30 min then diluted with water (15 ml). The reaction mixture was extracted with EtOAc, washed with saturated aqueous NaHCO₃, and dried over anhydrous MgSO₄. After removal of the solvent, the residue was purified by column chromatography on silica gel to provide ditert-butyl [4-(hydroxymethyl)-3-pyridyl] phosphate. MS (m/z) 317.9 [M+H]⁺.

### Example 70: Preparation of 4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl)pyridin-3-yl dihydrogen phosphate (70)

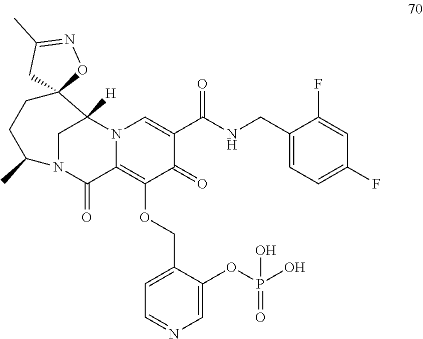 [SMILES: CC1=NO[C@@]2(CC[C@H](C)N3C[C@H]2n2cc(C(=O)NCc4ccc(F)cc4F)c(=O)c(OCc4ccncc4OP(=O)(O)O)c2C3=O)C1]

4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl)pyridin-3-yl dihydrogen phosphate was made following the same method as Example 41, except using ditert-butyl [4-(hydroxymethyl)-3-pyridyl] phosphate (Intermediate K) instead of di-tert-butyl (4-(hydroxymethyl)phenyl) phosphate in Step 1. MS (m/z) 674.1 [M+H]. 1H NMR (400 MHz, Methanol-d4) δ 8.82 (s, 1H), 8.60 (d, J=10.8 Hz, 2H), 8.45 (d, J=5.9 Hz, 1H), 7.51-7.34 (m, 1H), 7.10-6.80 (m, 214), 5.76-5.50 (m, 2H), 4.85-4.74 (m, 1H), 4.74-4.57 (m, 2H), 4.50 (s, 1H), 3.84 (t, J=1.9 Hz, 2H), 3.23-3.13 (m, 1H), 2.72 (d, J=17.9 Hz, 11H), 2.08 (s, 3H), 2.02-1.92 (m, 3H), 1.59 (d, J=9.9 Hz, 1H), 1.26 (d, J=6.7 Hz, 3H).

### Example 71: Preparation of 6-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl)pyridin-3-yl dihydrogen phosphate (71)

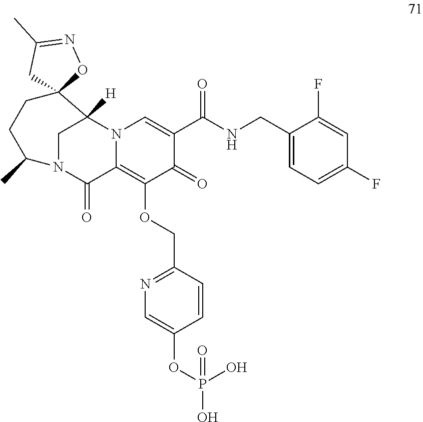 [SMILES: CC1=NO[C@@]2(CC[C@H](C)N3C[C@H]2n2cc(C(=O)NCc4ccc(F)cc4F)c(=O)c(OCc4ccc(OP(=O)(O)O)cn4)c2C3=O)C1]

6-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl)pyridin-3-yl dihydrogen phosphate was made following the same method as Example 41, except using ditert-butyl [6-(hydroxymethyl)-3-pyridyl]phosphate instead of di-tert-butyl (4-(hydroxymethyl)phenyl) phosphate. MS (m/z) 674.3 [M+H]⁺. 1H NMR (400 MHz, Methanol-d4) δ 8.83 (d, J=2.5 Hz, 1H), 8.66 (s, 1H), 8.36 (ddd, J=8.8, 2.6, 0.9 Hz, 1H), 8.05 (d, J=8.9 Hz, 1H), 7.46 (td, J=8.5, 6.3 Hz, 1H), 7.05-6.87 (m, 2H), 5.74-5.56 (m, 2H), 4.81-4.91 (m, 1H), 4.79-4.61 (m, 2H), 4.54 (d, J=2.2 Hz, 1H), 3.85 (qd, J=15.2, 2.3 Hz, 2H), 3.17 (dd, J=17.6, 1.2 Hz, 1H), 2.70 (dd, J=17.8, 1.2 Hz, 1H), 2.07 (s, 3H), 1.97 (dd, J=12.8, 6.7 Hz, 3H), 1.65-1.51 (m, 11H), 1.29 (d, J=6.7 Hz, 3H).

### Example 72: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((3-(phosphonooxy)pyridin-4-yl)methyl) carbonate (72)

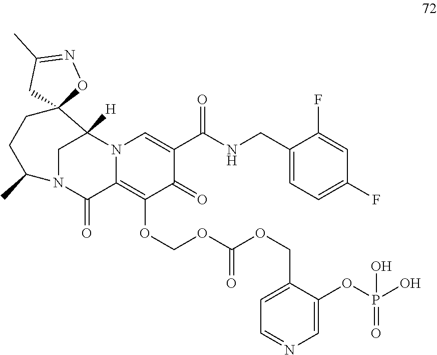 [SMILES: CC1=NO[C@@]2(CC[C@H](C)N3C[C@H]2n2cc(C(=O)NCc4ccc(F)cc4F)c(=O)c(OCOC(=O)OCc4ccncc4OP(=O)(O)O)c2C3=O)C1]

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((3-(phosphonooxy)pyridin-4-yl)methyl) carbonate was made following the same method as Example 37, except using ditert-butyl [4-(hydroxymethyl)-3-pyridyl]phosphate instead of di-tert-butyl (2-((methylamino)methyl)phenyl) phosphate in Step 2. MS (m/z) 748.0 [M+H]. 1H NMR (400 MHz, Methanol-d4) δ 8.82 (s, 11H), 8.56 (s, 11H), 8.50 (d, J=5.9 Hz, 1H), 8.11 (d, J=5.9 Hz, 1H), 7.50-7.33 (m, 11H), 7.01-6.78 (m, 2H), 6.05 (d, J=6.6 Hz, 1H), 5.77 (d, J=6.6 Hz, 1H), 5.54 (s, 2H), 4.72 (q, J=7.6 Hz, 1H), 4.63 (s, 2H), 4.43 (s, 1H), 3.77 (qd, J=15.2, 2.3 Hz, 2H), 3.23-3.09 (m, 1H), 2.79-2.62 (m, 1H), 2.07 (s, 3H), 1.93 (dd, J=12.9, 7.6 Hz, 3H), 1.48 (dd, J=14.1, 9.0 Hz, 1H), 1.20 (d, J=6.7 Hz, 3H).

### Example 73: Preparation of 4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)methyl)-3-(phosphonooxy)benzoic acid (73)

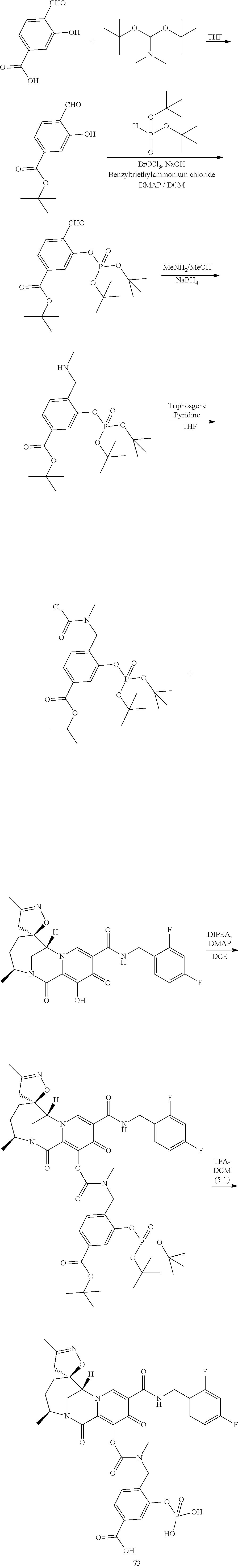

### Step 1: Preparation of tert-butyl 4-formyl-3-hydroxybenzoate

To a solution of 4-formyl-3-hydroxybenzoic acid (2 g, 12 mmol) in 10 mL dry THF (24 ml), 1,1-di-tert-butoxy-N,N-dimethylmethanamine (7.34 g, 36.1 mmol) was added at reflux. The mixture was stirred for 2 h at reflux. Then the solvent was removed and the residue was purified by column chromatography (ethyl acetate in hexane from 0% to 5%) to provide tert-butyl 4-formyl-3-hydroxybenzoate. ¹H NMR (400 MHz, Chloroform-d) δ 10.96 (s, 1H), 10.00 (d, J=0.7 Hz, 1H), 7.64 (d, J=1.3 Hz, 2H), 7.62-7.59 (m, 1H), 1.62 (s, 9H).

### Step 2: Preparation of tert-butyl 3-((di-tert-butoxyphosphoryl)oxy)-4-formylbenzoate

tert-butyl 3-((di-tert-butoxyphosphoryl)oxy)-4-formylbenzoate was synthesized following the same procedure as Example 31 synthesis, starting from tert-butyl 4-formyl-3-hydroxybenzoate.

### Step 3: Preparation of tert-butyl 3-((di-tert-butoxyphosphoryl)oxy)-4-((methylamino)methyl)benzoate

tert-butyl 3-((di-tert-butoxyphosphoryl)oxy)-4-((methylamino)methyl)benzoate was synthesized following the same procedure as Example 37 synthesis, starting from tert-butyl 3-((di-tert-butoxyphosphoryl)oxy)-4-formylbenzoate. MS (m/z) 430.814 [M+H].

### Step 4: Preparation of tert-butyl 4-(((chlorocarbonyl)(methyl)amino)methyl)-3-((di-tert-butoxyphosphoryl)oxy)benzoate

tert-butyl 4-(((chlorocarbonyl)(methyl)amino)methyl)-3-((di-tert-butoxyphosphoryl)oxy)benzoate was synthesized followed the same procedure as Example 38 synthesis, starting from tert-butyl 3-((di-tert-butoxyphosphoryl)oxy)-4-((methylamino)methyl)benzoate.

### Step 5: Preparation of tert-butyl 3-((di-tert-butoxyphosphoryl)oxy)-4-((((((3′S,5S,7′R)-0′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)methyl)benzoate

tert-butyl 3-((di-tert-butoxyphosphoryl)oxy)-4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)methyl)benzoate was synthesized following the same procedure as Example 38 synthesis, starting from tert-butyl 4-(((chlorocarbonyl)(methyl)amino)methyl)-3-((di-tert-butoxyphosphoryl)oxy)benzoate and (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide. MS (m/z) 942.626 [M+H]⁺.

### Step 6: Synthesis of 4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)methyl)-3-(phosphonooxy)benzoic acid

4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)methyl)-3-(phosphonooxy)benzoic acid was synthesized following the same procedure as Example 38 synthesis, starting from tert-butyl 3-((di-tert-butoxyphosphoryl)oxy)-4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)methyl)benzoate. MS (m/z) 774.7 [M+H]⁺. ¹H NMR (400 MHz, DMSO-d6) δ 10.24 (d, J=16.5 Hz, 1H), 8.82 (s, 1H), 7.89 (s, 1H), 7.73 (s, 2H), 7.51-7.40 (m, 1H), 7.34-7.16 (m, 1H), 7.09 (t, J=8.6 Hz, 1H), 4.88-4.41 (m, 7H), 3.74 (s, 1H), 3.05 (s, 2H), 2.89 (s, 2H), 2.69 (s, 1H), 1.95 (s, 3H), 1.82 (s, 3H), 1.24 (s, 1H), 1.18 (d, J=6.5 Hz, 3H).

### Example 74: Preparation of 4-(((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)methyl)-3-(phosphonooxy)benzoic acid (74)

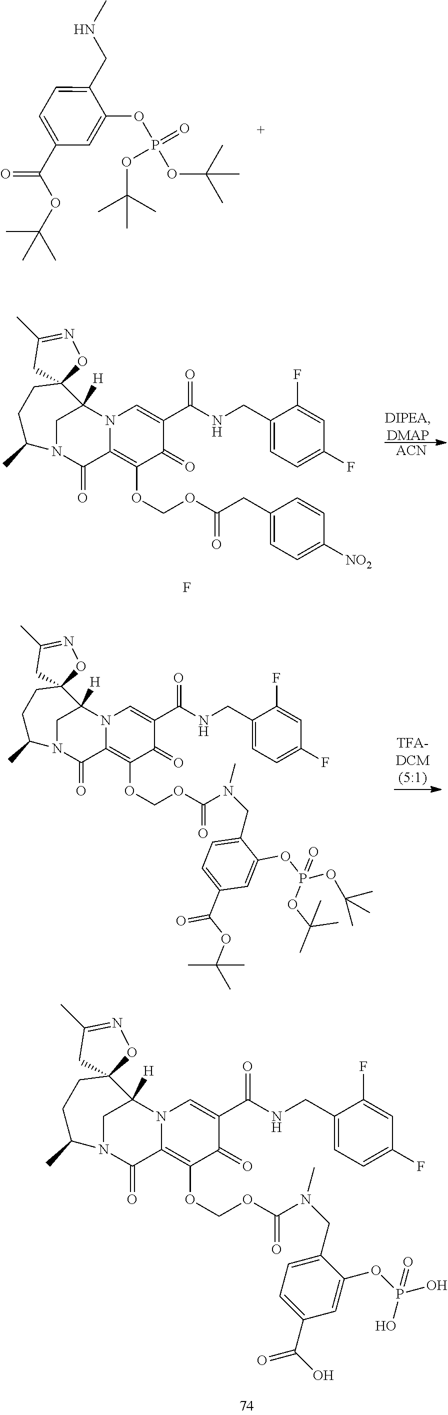

4-(((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)methyl)-3-(phosphonooxy)benzoic acid was synthesized following the same procedure as Example 37, starting from tert-butyl 3-((di-tert-butoxyphosphoryl)oxy)-4-((methylamino)methyl)benzoate and (((3′S,5S,7′R)-10′-((2,4-difluorobenzyl)carbamoyl)-3,3′-dimethyl-1′,11′-dioxo-1′,4′,5′,11′-tetrahydro-3′H,4H,77′H-spiro[isoxazole-5,6′-[2,7]methanopyrido[1,2-a][1,4]diazonin]-12′-yl)oxy)methyl 2-(4-nitrophenyl)acetate. MS (m/z) 804.88 [M+H]+. ¹H NMR (400 MHz, DMSO-d6) δ 10.33 (q, J=3.9, 2.2 Hz, 1H), 8.70 (s, 1H), 7.92-7.82 (m, 1H), 7.69 (s, 1H), 7.50-7.19 (m, 3H), 7.13-7.01 (m, 11H), 5.87 (d, J=6.4 Hz, 11H), 5.63 (d, J=6.3 Hz, 11H), 4.81-4.35 (m, 6H), 3.68 (s, 2H), 3.00 (d, J=17.5 Hz, 1H), 2.80 (d, J=13.5 Hz, 3H), 2.69-2.59 (m, 1H), 1.94 (d, J=3.2 Hz, 3H), 1.88-1.70 (m, 3H), 1.31 (d, J=11.2 Hz, 1H), 1.15-1.03 (m, 3H).

### Example 75: Preparation of 3-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)methyl)-4-(phosphonooxy)benzoic acid (75)

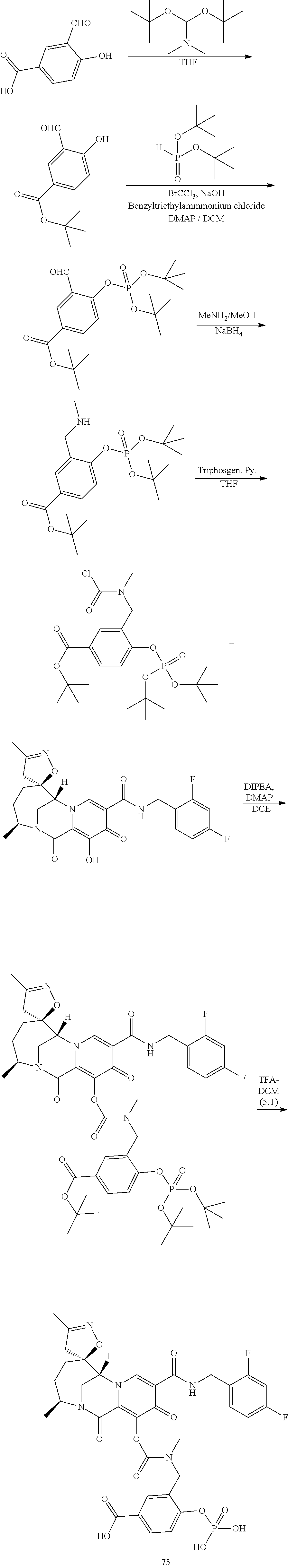

3-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)methyl)-4-(phosphonooxy)benzoic acid was synthesized following the same procedure as Example 73 synthesis, starting from 3-formyl-4-hydroxybenzoic acid. MS (m/z) 774.702 [M+H]+. ¹H NMR (400 MHz, DMSO-d6) δ 10.25 (d, J=18.7 Hz, 1H), 8.80 (s, 1H), 8.04 (s, 1H), 7.89 (d, J=8.5 Hz, 1H), 7.55-7.34 (m, 2H), 7.24 (td, J=9.9, 2.6 Hz, 1H), 7.17-7.02 (m, 11H), 4.77-4.64 (m, 2H), 4.55 (d, J=7.3 Hz, 4H), 3.69 (s, 3H), 3.02 (d, J=16.9 Hz, 2H), 2.90 (s, 1H), 2.66 (d, J=17.0 Hz, 1H), 1.95 (s, 3H), 1.80 (s, 3H), 1.22 (d, J=12.2 Hz, 1H), 1.16 (d, J=6.6 Hz, 3H).

### Example 76: Preparation of 3-(((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)methyl)-4-(phosphonooxy)benzoic acid (76)

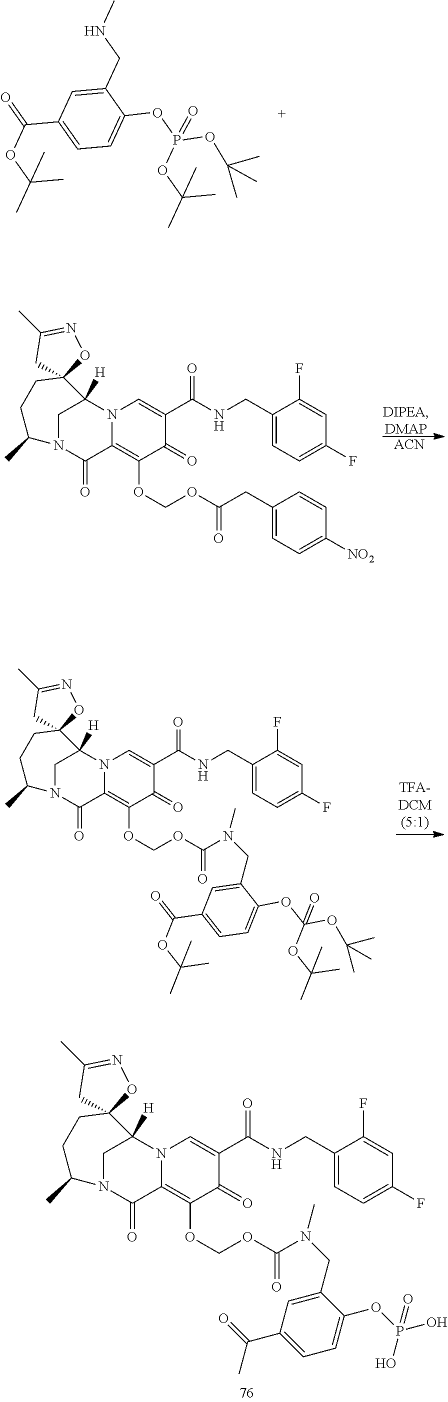

3-(((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)methyl)-4-(phosphonooxy)benzoic acid was synthesized following the same procedure as Example 74 synthesis (Step 1 and Step 2), starting from tert-butyl 4-((di-tert-butoxyphosphoryl)oxy)-3-((methylamino)methyl)benzoate and (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 2-(4-nitrophenyl)acetate. MS (m/z) 804.729 [M+H]⁺. ¹H NMR (400 MHz, DMSO-d6) δ 10.38-10.29 (m, 1H), 8.68 (s, 1H), 7.95-7.62 (m, 2H), 7.40 (dt, J=6.6, 3.2 Hz, 2H), 7.29-7.14 (m, 1H), 7.05 (dd, J=9.9, 7.4 Hz, 1H), 5.78 (dd, J=24.8, 6.3 Hz, 1H), 5.63 (td, J=6.1, 3.7 Hz, 1H), 4.70-4.41 (m, 6H), 3.72-3.65 (m, 2H), 3.01 (s, 1H), 2.80 (d, J=11.5 Hz, 3H), 2.62 (dd, J=17.6, 5.7 Hz, 1H), 1.94 (s, 3H), 1.74 (d, J=17.5 Hz, 3H), 1.27 (dd, J=11.7, 5.5 Hz, 1H), 1.16-1.03 (m, 3H).

### Example 77: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((1S,2S)-2-(phosphonooxy)cycloheptyl) carbonate (77)

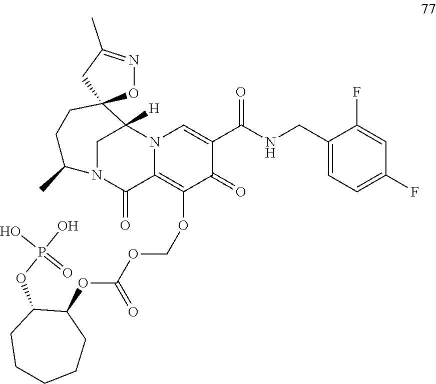 [SMILES: CC1=NO[C@@]2(CC[C@H](C)N3C[C@H]2n2cc(C(=O)NCc4ccc(F)cc4F)c(=O)c(OCOC(=O)O[C@H]4CCCCC[C@@H]4OP(=O)(O)O)c2C3=O)C1]

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((1S,2S)-2-(phosphonooxy)cycloheptyl) carbonate was made following the same method as Example 44, except in Step 1, (1S,2S)-cyclohexane-1,2-diol was replaced by (1S,2S)-cycloheptane-1,2-diol. MS (m/z) 753.0 [M+H]+. ¹H NMR (400 MHz, DMSO) δ 10.28 (d, J=5.4 Hz, 1H), 8.88-8.57 (m, 1H), 7.57-7.33 (m, 1H), 7.33-7.22 (m, 1H), 7.22-6.89 (m, 1H), 5.92-5.85 (m, 1H), 5.78-5.68 (m, 1H), 5.57-5.52 (m, 1H), 4.72-4.47 (m, 5H), 4.37-4.21 (m, 1H), 3.76-3.63 (m, 2H), 3.00 (d, J=17.2 Hz, 1H), 2.66-2.59 (m, 1H), 1.95 (s, 4H), 1.90-1.21 (m, 14H), 1.16 (d, J=6.6 Hz, 3H).

### Preparation of Intermediates L and M: 1-benzyl 2-(tert-butyl) (1R,2R,3S)-3-(((di-tert-butoxyphosphoryl)oxy)methyl)cyclopropane-1,2-dicarboxylate (Intermediate L) and 1-benzyl 2-(tert-butyl) (1S,2S,3R)-3-(((di-tert-butoxyphosphoryl)oxy)methyl)cyclopropane-1,2-dicarboxylate (Intermediate M)

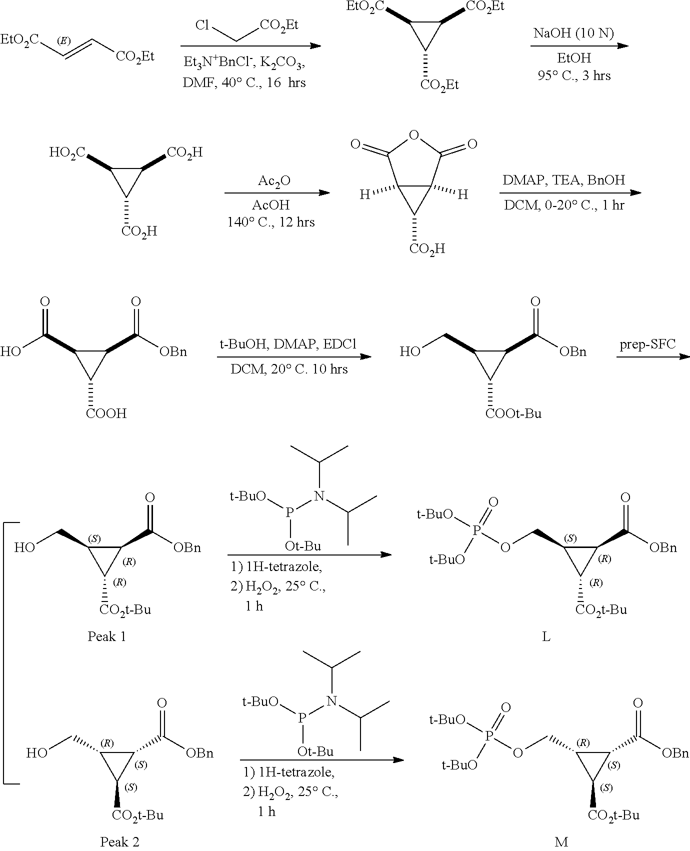

### Step 1: Preparation of triethyl (r)-cyclopropane-1,2,3-tricarboxylate

Added diethyl fumarate (2.00 kg, 11.6 mol, 1.00 eq), ethyl 2-chloroacetate (2.60 kg, 15.7 mol, 1.35 eq) in DMF (10.0 L), then added K₂CO₃ (3.52 kg, 25.5 mol, 2.20 eq), Et₃N⁻BnCl⁻ (2.64 kg, 1.6 mol, 1.00 eq) to the mixture and stirred at 40° C. for 16 hrs. Quenched the reaction with H₂O (30.0 L) and extract the mixture with MTBE (12.0 L×3). Dry over the combined organic layers with Na₂SO₄, filter and concentrate the filtrate under reduced pressure. The residue was purified with flash silica gel chromatography (ISCO®; 3.30 kg SepaFlash® Silica Flash Column, Eluent of 0˜10% Ethyl acetate/Petroleum ether gradient @200 mL/min) to obtain methyl (r)-cyclopropane-1,2,3-tricarboxylate.

### Step 2: Preparation of (r)-cyclopropane-1,2,3-tricarboxylic acid

Added triethyl (r)-cyclopropane-1,2,3-tricarboxylate (1.05 kg, 3.87 mol, 1.00 eq) in EtOH (525 mL), then added NaOH (10.0 M, 500 g, 3.30 eq) to the mixture and stirred at 95° C. for 3 hrs. Cooled the reaction mixture to 0° C., added HCl (4 M, 2.00 L) dropwise to adjust pH=1. Concentrated the reaction mixture to remove water. Then added toluene and continued to concentrate until most of water was removed, slurried the residue with MeOH (2.00 L) and filter the mixture, concentrate the filtrate in vacuum to obtain (r)-cyclopropane-1,2,3-tricarboxylic acid (601 g, crude).

### Step 3: Preparation of (1R,5S,6s)-2,4-dioxo-3-oxabicyclo[3.1.0]hexane-6-carboxylic acid

Added (r)-cyclopropane-1,2,3-tricarboxylic acid (601 g, 3.44 mol, 1.00 eq), Ac2O (704 g, 6.88 mol, 2.00 eq) in AcOH (1.14 L) and stirred at 140° C. for 12 hrs. Then concentrated in vacuum to remove acetic acid, followed by trituration of the residue with EtOAc (1.00 L) at 0-5° C., and stirred for 0.5 hr. Then it was filtered, washed with EtOAc (500 mL) and dried under vacuum to obtain (R,5S,6s)-2,4-dioxo-3-oxabicyclo[3.1.0]hexane-6-carboxylic acid.

### Step 4: Preparation of (1R,2R)-3-((benzyloxy)carbonyl)cyclopropane-1,2-dicarboxylic acid

Added (1R,5S,6s)-2,4-dioxo-3-oxabicyclo[3.1.0]hexane-6-carboxylic acid (430 g, 2.81 mol, 1.0) eq), BnOH (292 g, 2.81 mol, 1.00 eq) in DCM (4.30 L), then to the mixture was added DMAP (33.6 g, 0.281 mol, 0.10 eq) and TEA (560 g, 5.62 mol, 2.00 eq) at 0° C., stirred it at 20° C. for 1 hr. Then the reaction was poured to NaHCO₃ (3×1.00 L) and adjust with 4M HCl to pH=1-2. The organic layers was dried over Na₂SO₄, filtered and concentrated under reduced pressure to obtain (1R,2R)-3-((benzyloxy)carbonyl)cyclopropane-1,2-dicarboxylic acid.

### Step 5: Preparation of (1S,2R,3R)-2-((benzyloxy)carbonyl)-3-(tert-butoxycarbonyl)cyclopropane-1-carboxylic acid

Added (1R,2R)-3-((benzyloxy)carbonyl)cyclopropane-1,2-dicarboxylic acid (267 g, 181 mmol, 1.00 eq), t-BuOH (48.5 g, 217 mmol, 1.20 eq) in DCM (1.86 L). Then added DMAP (6.65 g, 18.1 mol, 0.10 eq), EDCI (52.2 g, 272 mmol, 1.50 eq) to the mixture and stirred at 20° C. for 10 hrs. Then the reaction mixture was poured into H₂O (300 mL), then extracted the mixture with EtOAc (300 mL×2). The combined organic layers were dried over Na₂SO₄, filtered and the mixture was concentrated under reduced pressure to obtain (1S,2R,3R)-2-((benzyloxy)carbonyl)-3-(tert-butoxycarbonyl)cyclopropane-1-carboxylic acid. 1H NMR: (400 MHz, CDCl₃-d) δ 7.42-7.30 (m, 5H), 5.18-5.14 (m, 2H), 2.79-2.71 (m, 1H), 2.64-2.56 (m, 1H), 2.55-2.46 (m, 1H), 1.44 (d, J=18.8 Hz, 9H).

### Step 6: Preparation of 1-benzyl 2-(tert-butyl) (1R,2R,3S)-3-(hydroxymethyl)cyclopropane-1,2-dicarboxylate

Added (1S,2R,3R)-2-((benzyloxy)carbonyl)-3-(tert-butoxycarbonyl)cyclopropane-1-carboxylic acid (91.1 g, 31.2 mmol, 1.00 eq) in THF (450 mL), cooled to 0-5° C., added dropwise BH3-THF (108 g, 156 mmol, 5.00 eq) to the mixture at 0-5° C., and stirred at 20° C. for 12 hrs. Then added MeOH (500 mL) at 0-5° C., and concentrated under reduced pressure to give a residue. The residue was purified with flash silica gel chromatography (ISCO®; 100 g SepaFlash®, Silica Flash Column, Eluent of 0-100% Ethyl acetate/Petroleum ether gradient @r 20.0 mL/min) to obtain 1-benzyl 2-(tert-butyl) (1R,2R,3S)-3-(hydroxymethyl)cyclopropane-1,2-dicarboxylate. ¹H NMR: (CDCl₃-d 400 MHz) δ 7.43-7.33 (m, 5H), 5.17 (d, J=0.8 Hz, 2H), 3.97 (dd, J=5.2, 12.0 Hz, 1H), 3.79 (dd, J=7.8, 12.0 Hz, 1H), 2.38-2.25 (m, 2H), 2.13-2.01 (m, 1H), 1.45 (s, 9H).

### Step 7: Perspiration of 1-benzyl 2-(tert-butyl) (1R,2R,3S)-3-(hydroxymethyl)cyclopropane-1,2-dicarboxylate & 1-benzyl 2-(tert-butyl) (1S,2S,3R)-3-(hydroxymethyl)cyclopropane-1,2-dicarboxylate

The mixture of 1-benzyl 2-(tert-butyl) (1R,2R,3S)-3-(hydroxymethyl)cyclopropane-1,2-dicarboxylate and 1-benzyl 2-(tert-butyl) (1S,2S,3R)-3-(hydroxymethyl)cyclopropane-1,2-dicarboxylate was purified by prep-SFC (column: DAICEL CHIRALPAK IG (250 mm*50 mm, 10 um); mobile phase: [0.1% NH3H2O ETOH]; B %: 25%-25%, 9.3 min; column: DAICEL CHIRALPAK IG (250 mm*50 mm, 10 um); mobile phase: [0.1% NH3H2O ETOH]; B %: 30%-30%, 2.5 min) to obtain 1-benzyl 2-(tert-butyl) (1R,2R,3S)-3-(hydroxymethyl)cyclopropane-1,2-dicarboxylate (Peak1) and (1S,2S,3R)-3-(hydroxymethyl)cyclopropane-1,2-dicarboxylate (Peak2).

Peak 1: ¹H NMR: (CHCl₃-d 400 MHz): δ 7.47-7.29 (m, 5H), 5.17 (s, 2H), 3.97 (dd, J=5.2, 12.0 Hz, 1H), 3.79 (dd, J=7.8, 12.0 Hz, 1H), 2.36-2.22 (m, 2H), 2.14-2.00 (m, 1H), 1.51-1.41 (m, 9H).

Peak 2: ¹H NMR: (CHCl₃-d 400 MHz): δ 7.43-7.34 (m, 5H), 5.17 (s, 2H), 3.97 (dd, J=5.2, 12.0 Hz, 1H), 3.79 (dd, J=7.8, 12.0 Hz, 1H), 2.42-2.21 (m, 2H), 2.16-2.00 (m, 2H), 1.46-1.44 (m, 9H).

### Step 8: Preparation of 1-benzyl 2-(tert-butyl) (1R,2R,3S)-3-(((di-tert-butoxyphosphoryl)oxy)methyl)cyclopropane-1,2-dicarboxylate (Intermediate L)

Added 1-benzyl 2-(tert-butyl) (1R,2R,3S)-3-(hydroxymethyl)cyclopropane-1,2-dicarboxylate (11.0 g, 35.9 mmol, 1.00 eq), di-tert-butyl diisopropylphosphoramidite (21.7 g, 35.9 mmol, 1.00 eq) and 1H-tetrazole (6.00 g, 85.6 mmol, 2.20 eq) in THF (110 mL) and stirred at 25° C. for 1 hr. Then added H₂O₂ (17.7 g, 156 mmol, 4.00 eq) dropwise to the mixture and stirred at 25° C. for 1 hr. The reaction was quenched with Na₂SO₃ (300 mL) slowly and extracted with DCM (100 mL×2). The combined organic layers were dried over Na₂SO₄, filtered and the mixture was concentrated under reduced pressure. The residue was purified by prep-HPLC (column: Welch Xtimate C18 250*70 mm #10 um; mobile phase: [water (ammonia hydroxide v/v)-ACN]; B %: 55%-85%, 20 min) to obtain 1-benzyl 2-(tert-butyl) (1R,2R,3S)-3-(((di-tert-butoxyphosphoryl)oxy)methyl)cyclopropane-1,2-dicarboxylate (Intermediate L). LCMS (m/z): 331.0 [M-3t-Bu+H]⁺. ¹H NMR: (CHCl₃-d 400 MHz): δ 7.42-7.31 (m, 51H), 5.25-5.07 (m, 2H), 4.36-4.23 (m, 1H), 4.10 (td, J=8.0, 11.0 Hz, 1H), 3.48-3.32 (m, 1H), 2.42-2.32 (m, 1H), 2.29-2.13 (m, 2H), 2.02 (s, 1H), 1.48 (s, 16H), 1.44 (s, 8H), 1.22 (d, J=6.8 Hz, 1H).

### Step 9: Preparation of 1-benzyl 2-(tert-butyl) (1S,2S,3R)-3-(((di-tert-butoxyphosphoryl)oxy)methyl)cyclopropane-1,2-dicarboxylate (Intermediate M)

Added 1-benzyl 2-(tert-butyl) (1S,2S,3R)-3-(hydroxymethyl)cyclopropane-1,2-dicarboxylate (10.0 g, 35.9 mmol, 1.00 eq), di-tert-butyl diisopropylphosphoramidite (19.7 g, 35.9 mmol, 1.00 eq) and 1H-tetrazole (5.45 g, 79.0 mmol, 2.20 eq) in THF (100 mL) and stirred at 25° C. for 1 hr. Then added H₂O₂ (16.1 g, 144 mmol, 4.00 eq) dropwise to the mixture and stirred at 25° C. for 1 hr. The reaction was quenched with Na₂SO₃ (300 mL) slowly and extracted with DCM (100O mL x 2). The combined organic layer was dried over Na₂SO₄, filtered and concentrated under reduced pressure. The residue was purified by prep-HPLC (column: Welch Xtimate C18 250*70 mm #10 um; mobile phase: [water (ammonia hydroxide v/v)-ACN]; B %: 55%-85%, 20 min) to obtain 1-benzyl 2-(tert-butyl) (1S,2S,3R)-3-(((di-tert-butoxyphosphoryl)oxy)methyl)cyclopropane-1,2-dicarboxylate (Intermediate M). LCMS (m/z): 331.0 [M-3t-Bu+H]. 1H NMR: (CHCl3-d 400 MHz): δ 7.40-7.27 (m, 5H), 5.19-5.10 (m, 2H), 4.31-4.28 (m, 1H), 4.10 (td, J=8.0, 11.0 Hz, 1H), 3.42-3.34 (m, 1H), 2.38-2.34 (m, 1H), 2.25-2.18 (m, 2H), 2.02 (s, 1H), 1.48 (s, 16H), 1.44 (s, 8H), 1.22 (d, J=6.8 Hz, 1H).

### Example 78: Preparation of (1R,2R,3R)-2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)-3-((phosphonooxy)methyl)cyclopropane-1-carboxylic acid (78)

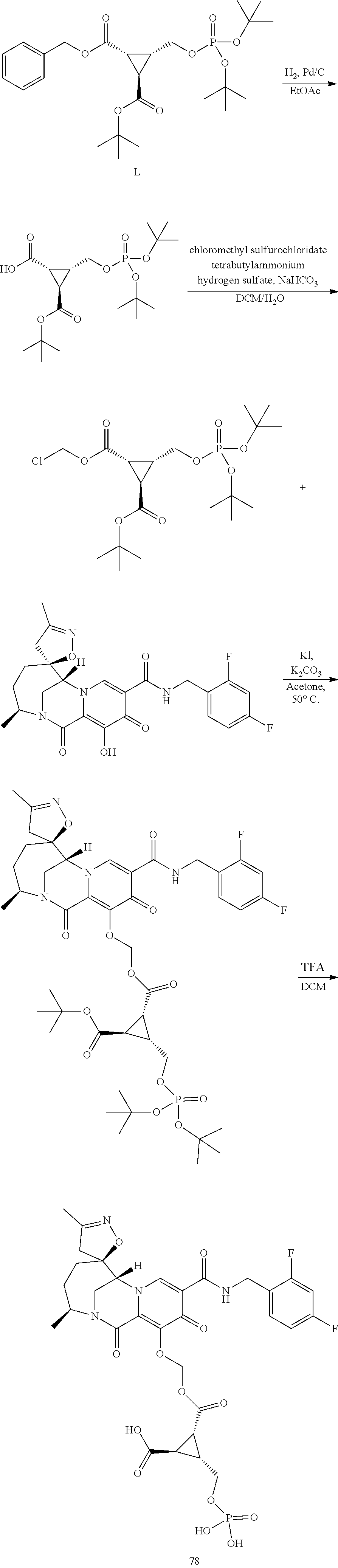

### Step 1: Preparation of (1R,2R,35)-2-(tert-butoxy carbonyl)-3-(((di-tert-butoxyphosphoryl)oxy)methyl)cyclopropane-1-carboxylic acid

To a solution of 1-benzyl 2-(tert-butyl) (1R,2R,3S)-3-(((di-tert-butoxyphosphoryl)oxy)methyl)cyclopropane-1,2-dicarboxylate (Intermediate L, 1000 mg, 2 mmol) in EtOAc (10 mL) was added 10% Pd/C (427 g, 0.4 mmol). The flask was evacuated and backfilled with hydrogen gas (2×), then sparged with hydrogen for 2 min. The reaction mixture was left to stir under a hydrogen balloon atmosphere overnight. The reaction mixture was filtered, rinsed with EtOAc, and concentrated to afford crude product (1R,2R,3S)-2-(tert-butoxycarbonyl)-3-(((di-tert-butoxyphosphoryl)oxy)methyl)cyclopropane-1-carboxylic acid. MS (m/z) 409.0 [M+H]⁺.

### Step 2: Preparation of 1-(tert-butyl) 2-(chloromethyl) (1R,2R,3R)-3-(((di-tert-butoxyphosphoryl)oxy)methyl)cyclopropane-1,2-dicarboxylate

A biphasic mixture of the (1R,2R,3S)-2-(tert-butoxycarbonyl)-3-(((di-tert-butoxyphosphoryl)oxy)methyl)cyclopropane-1-carboxylic acid (0.56 g, 1.37 mmol), tetrabutylammonium hydrogen sulfate (46 mg, 0.14 mmol), and sodium bicarbonate (0.93 g, 11 mmol) in water (5 mL) and DCM (5 mL) was cooled to 0° C. While stirring, chloromethyl chlorosulfate (0.45 g, 2.75 mmol) was added dropwise. The reaction mixture was allowed to stir at RT overnight. Brine was added and the aqueous phase was extracted with DCM (3×). The combined organic phase was concentrated. The crude product was purified by column chromatography (0-100% EtOAc/hexanes) to afford 1-(tert-butyl) 2-(chloromethyl) (1R,2R,3R)-3-(((di-tert-butoxyphosphoryl)oxy)methyl)cyclopropane-1,2-dicarboxylate. MS (m/z) 456.8 [M+H]⁺.

### Step 3: Preparation of 1-(tert-butyl) 2-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) (1R,2R,3R)-3-(((di-tert-butoxyphosphoryl)oxy)methyl)cyclopropane-1,2-dicarboxylate

Intermediate B (161 mg, 0.33 mmol), K₂CO₃ (91 mg, 0.66 mmol) and KI (71 mg, 0.43 mmol) were mixed in acetone (3 mL) at room temperature. Then a solution of 1-(tert-butyl) 2-(chloromethyl) (1R,2R,3R)-3-(((di-tert-butoxyphosphoryl)oxy)methyl)cyclopropane-1,2-dicarboxylate (302 mg, 0.66 mmol) in acetone (1 mL) was added. The reaction mixture was heated at 50 degree for 3 h. The solvent was evaporated and then dissolved in DCM, washed with aqueous saturated NH₄Cl (10 mL). The organic layer was separated and concentrated to dryness. The residue was purified with silica gel column eluted with 80% MeOH/DCM to afford 1-(tert-butyl) 2-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) (1R,2R,3R)-3-(((di-tert-butoxyphosphoryl)oxy)methyl)cyclopropane-1,2-dicarboxylate. MS (m/z) 906.8 [M+H]⁺.

### Step 4: Preparation of (1R,2R,3R)-2-(((((3′S,5S,7′R)-10′-((2,4-difluorobenzyl)carbamoyl)-3,3′-dimethyl-1′,11′-dioxo-1′,4′,5′,11′-tetrahydro-3′H,4H7′H-spiro[isoxazole-5,6′-[2,7]methanopyrido[1,2-a][1,4]diazonin]-12′-yl)oxy)methoxy)carbonyl)-3-((phosphonooxymethyl)cyclopropane-1-carboxylic acid (78)

1-(tert-butyl) 2-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) (1R,2R,3R)-3-(((di-tert-butoxyphosphoryl)oxy)methyl)cyclopropane-1,2-dicarboxylate (100 mg, 0.11 mmol) was dissolved in DCM (3 mL), to the above solution was added TFA (0.3 ml), The reaction mixture was stirred at RT for 5 h. The solvent was evaporated, the residue was purified with C-18 column on Combi-Flash to afford (1R,2R,3R)-2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)-3-((phosphonooxy)methyl)cyclopropane-1-carboxylic acid. MS (m/z) 738.7 [M+H]⁺. 1H NMR (400 MHz, Methanol-d4) δ 8.58 (s, 1H), 7.45 (td, J=8.4, 6.3 Hz, 1H), 7.06-6.83 (m, 2H), 5.96-5.70 (m, 2H), 4.88-4.76 (m, 1H), 4.73-4.56 (m, 2H), 4.49 (d, J=2.3 Hz, 1H), 4.35-4.09 (m, 2H), 3.92-3.69 (m, 2H), 3.16 (d, J=17.9 Hz, 1H), 2.76-2.59 (m, 1H), 2.43-2.15 (m, 3H), 2.06 (s, 3H), 2.03-1.85 (m, 3H), 1.62-1.42 (m, 1H), 1.27 (d, J=6.7 Hz, 3H).

### Example 79: Preparation of (1S,2S,3S)-2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)-3-((phosphonooxy)methyl)cyclopropane-1-carboxylic acid (79)

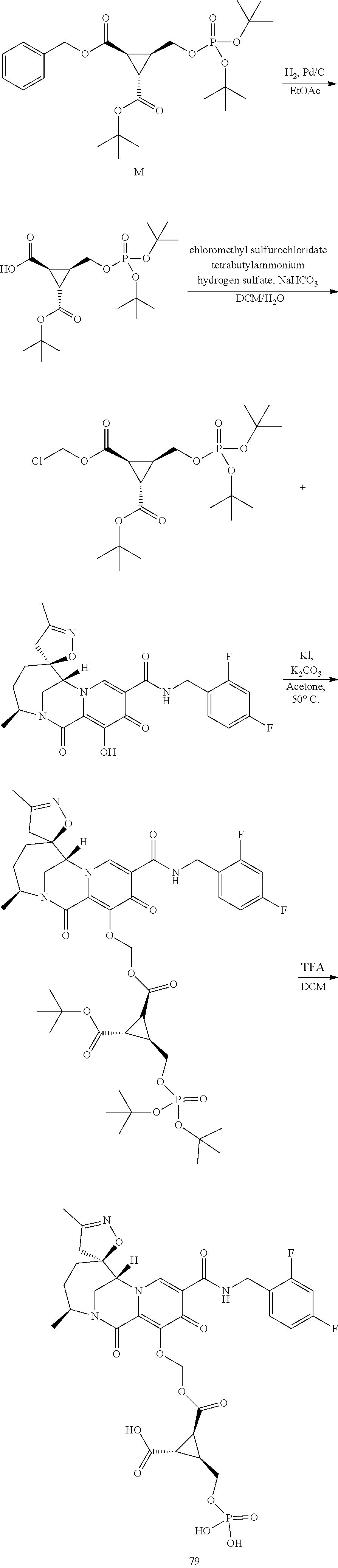

(1S,2S,3S)-2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)-3-((phosphonooxy)methyl)cyclopropane-1-carboxylic acid was synthesized following the same procedure as Steps 1-4 of Example 78, except using 1-benzyl 2-(tert-butyl) (1S,2S,3R)-3-(((di-tert-butoxyphosphoryl)oxy)methyl)cyclopropane-1,2-dicarboxylate (Intermediate M) instead of 1-benzyl 2-(tert-butyl) (1R,2R,3S)-3-(((di-tert-butoxyphosphoryl)oxy)methyl)cyclopropane-1,2-dicarboxylate (Intermediate L). MS (m/z) 738.8 [M+H]⁺. 1H NMR (400 MHz, Methanol-d4) δ 8.58 (s, 1H), 7.45 (td, J=8.5, 6.4 Hz, 1H), 7.08-6.85 (m, 2H), 6.08 (d, J=6.5 Hz, 1H), 5.59 (d, J=6.5 Hz, 1H), 4.82 (q, J=8.0, 7.2 Hz, 1H), 4.74-4.56 (m, 2H), 4.48 (d, J=2.3 Hz, 1H), 4.24 (dt, J=11.4, 7.1 Hz, 1H), 4.14 (ddd, J=11.4, 8.5, 7.3 Hz, 1H), 3.80 (d, J=2.3 Hz, 2H), 3.16 (d, J=17.8 Hz, 1H), 2.76-2.64 (m, 1H), 2.37 (dd, J=9.3, 4.8 Hz, 1H), 2.30-2.11 (m, 2H), 2.06 (s, 3H), 1.94 (q, J=9.0 Hz, 3H), 1.63-1.44 (m, 1H), 1.25 (d, J=6.7 Hz, 3H).

### Intermediate N: Preparation of (1R,2S)-2-(((tert-butoxycarbonyl)amino)methyl)cyclobutane-1-carboxylic acid

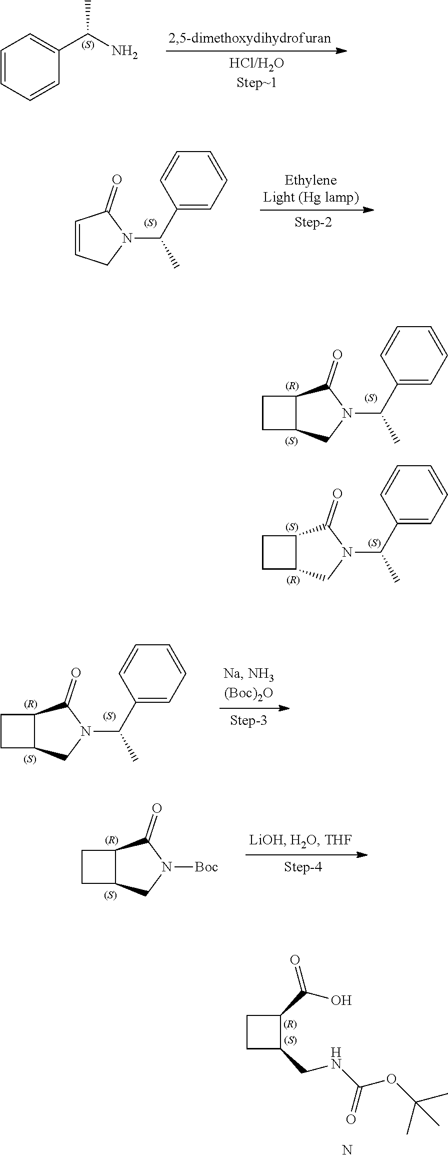

### Step 1: Preparation of (S)-1-(1-phenylethyl)-1,5-dihydro-2H-pyrrol-2-one

The (S)-1-phenylethan-1-amine (4.225 mmol) reacted with a mixture of 2,5-dimethoxydihydrofuran (0.500 g, 3.841 mmol) and conc. HCl (0.10 mL, 1.207 mmol) as a solution in H₂O (3.0 mL) at RT for 6 h. After the end of reaction, saturated solution of NaHCO₃ was added until pH 6 and the mixture was extracted with EtOAc (3×50 mL). The organic layers were dried with Na₂SO₄, filtered, concentrated in vacuum and crude product was then purified by column chromatography eluted with EtOAc to afford (S)-1-(1-phenylethyl)-1,5-dihydro-2H-pyrrol-2-one.

### Step 2: Preparation of (1R,5S)-3-((S)-1-phenylethyl)-3-azabicyclo[3.2.0]heptan-2-one and (1S,5R)-3-((S)-1-phenylethyl)-3-azabicyclo[3.2.0]heptan-2-one

A solution of (S)-1-(1-phenylethyl)-1,5-dihydro-2H-pyrrol-2-one (2.00 g; 10.7 mmol) in acetone (1 L) in a cylindrical water-cooled photochemical reactor was degassed with an argon stream for 30 min then saturated with an ethylene stream for 30 min. The solution was then irradiated for 9 h with a 400 W medium-pressure mercury vapor lamp fitted with a Pyrex filter while a slow stream of ethylene was maintained. The solvent was evaporated under reduced pressure and the residue was chromatographed (Combiflash) using EtOAc/petroleum ether as eluent (gradient 40:601t 100:0) to obtain (1R,5S)-3-((S)-1-phenylethyl)-3-azabicyclo[3.2.0]heptan-2-one [a]_{D}²⁰ −189 (c 0.99, CHCl₃); Rf 0.42 (EtOAc/petroleum ether 50:50) and (1S,5R)-3-((S)-1-phenylethyl)-3-azabicyclo[3.2.0]heptan-2-one [a]_{D}²⁰ −123 (c 0.97, CHCl₃); Rf 0.33 (EtOAc/petroleum ether 50:50).

### Step 3: Preparation of tert-butyl (1R,5S)-2-oxo-3-azabicyclo[3.2.0]heptane-3-carboxylate

Ammonia (˜100 mL) was condensed and retained at −33° C. in a three-neck flask fitted with a cold-finger condenser (−78° C.). Small sticks of sodium (484 mg; 21.0 mmol) were washed in petroleum ether and added to the flask. A solution of (1R,5S)-3-((S)-1-phenylethyl)-3-azabicyclo[3.2.0]heptan-2-one (647 mg; 3.0 mmol) and t-BuOH (643 mL; 6.6 mmol; 2.2 equiv) in THF (12 mL) was added slowly and the mixture was stirred for 45 min. Solid NH₄Cl was then added until the mixture became colorless. Cooling was removed and the ammonia was allowed to evaporate, then water (2 mL) and 1 M HCl (30 mL) were added slowly, successively. The aqueous phase was extracted with CH₂Cl₂ (6×20 mL) and the combined extracts dried over Na₂SO₄. After filtration and evaporation of the solvent under reduced pressure, the residue (379 mg) was dissolved in acetonitrile (25 mL). This solution was cooled to 0° C., and DMAP (41.7 mg; 0.31 mmol) and Boc₂O (1.5 g; 6.8 mmol) were added successively. The solution was then allowed to warm to rt and stirred overnight. The solvent was evaporated under reduced pressure and the crude product chromatographed (Combiflash) using Et₂O/petroleum ether as eluent (gradient 5:95 to 80:20) to obtain tert-butyl (1R,5S)-2-oxo-3-azabicyclo[3.2.0]heptane-3-carboxylate. MS (CI—NH₃) m/z 229 [MH+NH₃]⁺, 212 [MH]⁺. ¹H NMR (CDCl₃; 360 MHz) δ (ppm) 1.55 (s, 9H), 1.93-2.08 (m, 1H), 2.08-2.21 (m, 1H), 2.28-2.41 (m, 1H), 2.41-2.58 (m, 1H), 2.91 (q, J=7 Hz, 1H), 3.05-3.15 (m, 1H), 3.62 (d, J=11 Hz, 1H), 3.79 (dd, J=11 Hz and 7 Hz, 1H); ¹³C NMR (CDCl₃; 90 MHz) d (ppm) 23.6, 25.7, 27.7, 28.5, 42.3, 52.6, 82.4, 150.3, 177.3.

### Step 4: Preparation of (1R,2S)-2-(((tert-butoxycarbonyl)amino)methyl)cyclobutane-1-carboxylic acid

A solution of tert-butyl (1R,5S)-2-oxo-3-azabicyclo[3.2.0]heptane-3-carboxylate (150 mg; 0.71 mmol) in THF (7 mL) and H₂O (7 mL) was treated with solid LiOH (170 mg; 7.1 mmol) and the mixture was stirred at rt for 24 h. The THF was evaporated under reduced pressure. The remaining solution was digested with EtOAc (20 mL) and 1 M oxalic acid (20 mL). The organic layer was collected and the aqueous phase was extracted with more EtOAc (5×20 mL). The combined organic extracts were dried over MgSO₄. The solution was filtered and the solvent evaporated under reduced pressure to provide (1R,2S)-2-(((tert-butoxycarbonyl)amino)methyl)cyclobutane-1-carboxylic acid (Intermediate N). MS (CI—NH₃) m/z 247 [MH+NH_{3], 230} [MH]+; HRMS (ESI): C₁₁H₁₉NNaO₄ requires m/z 252.1206 [M+Na]⁺; found 252.1. ¹H NMR (CDCl₃; 360 MHz) δ (ppm) 1.47 (s, 9H), 1.73-1.90 (m, 1H), 2.00-2.12 (m, 2H), 2.28-2.39 (m, 1H), 2.80-2.98 (m, 1H), 3.20-3.45 (m, 3H), 4.91 (br s, 1H), 9.70 (br s, 1H).

### Example 80: Preparation of N-(((1S,2R)-2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)cyclobutyl)methyl)-N-(((phosphonooxy)methoxy)carbonyl)glycine (80)

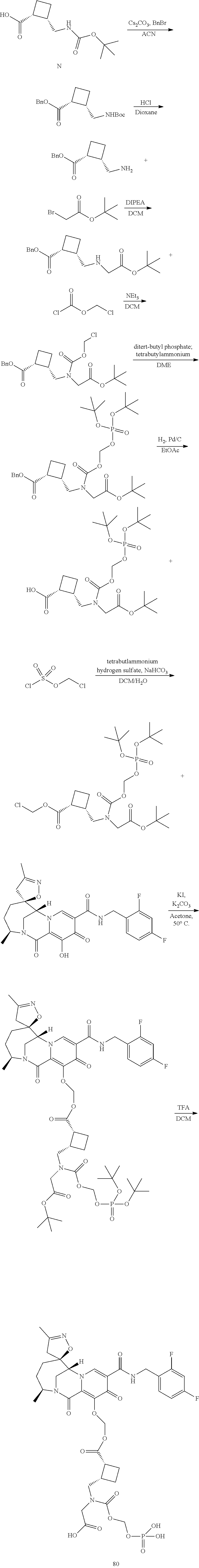

### Step 1: Preparation of benzyl (1R,2S)-2-[(tert-butoxycarbonylamino)methyl]cyclobutanecarboxylate

(1R,2S)-2-[(tert-butoxycarbonylamino)methyl]cyclobutanecarboxylic acid (Intermediate N, 2 g, 8.72 mmol) was dissolved in DMF (10 mL), to the solution was added Cs₂CO₃ (3.4 g, 10.5 mmol) and BnBr (1.79 g, 10.5 mmol) and stirred at RT overnight. The reaction mixture was then filtered and concentrated. The residue was purified with Combi-Flash column to afford benzyl (1R,2S)-2-[(tert-butoxycarbonylamino)methyl]cyclobutanecarboxylate. MS (m/z) 264.0 [M-isobutene]⁺.

### Step 2: Preparation of benzyl (JR 2S)-2-[(tert-butoxycarbonylamino)methyl]cyclobutanecarboxylate

Benzyl (1R,2S)-2-[(tert-butoxycarbonylamino)methyl]cyclobutanecarboxylate (2.4 g, 7.5 mmol) was treated with 4N HCl (30 mL) in dioxane (5 mL). After stirring for 1 h, the reaction mixture was concentrated to afford benzyl (1R,2S)-2-[(tert-butoxycarbonylamino)methyl]cyclobutanecarboxylate. MS (m/z) 220.0 [M+H]⁺.

### Step 3: Preparation of benzyl (1R,2S)-2-[[(2-tert-butoxy-2-oxo-ethyl)amino]methyl]cyclobutanecarboxylate

To a stirred suspension of benzyl (1R,2S)-2-[(tert-butoxycarbonylamino)methyl]cyclobutanecarboxylate (2.3 g, 7.2 mmol) in CH₂C₁₋₂ (50 mL) at 0° C. was added DIPEA (2.8 g, 21 mmol), t-butyl bromoacetate (0.7 g, 3.6 mmol) in CH₂Cl₂ (100 mL) was added dropwise over 30 min, then warmed to rt. The reaction mixture was then washed with 0.5M aq. HCl solution and brine. The organic layer was dried over Na₂SO₄, filtered, and concentrated. The residue was purified with Combi-Flash column to afford benzyl (1R,2S)-2-[[(2-tert-butoxy-2-oxo-ethyl)amino]methyl]cyclobutanecarboxylate. MS (m/z) 334.10 [M+H]⁺.

### Step 4: Preparation of benzyl (1R,2S)-2-[[(2-tert-butoxy-2-oxo-ethyl)-(chloromethoxycarbonyl)amino]methyl]cyclobutanecarboxylate

To a solution of benzyl (1R,2S)-2-[[(2-tert-butoxy-2-oxo-ethyl)amino]methyl]cyclobutanecarboxylate (776 mg, 2.3 mmol) in DCM (15 mL) at 0° C. was added NEt₃ (0.35 g, 3.5 mmol), followed by addition of chloromethyl chloroformate (0.45 g, 3.5 mmol) dropwise. After stirring at 0° C. for 10 min. the reaction was washed with brine. The organic layer was dried over anhydrous MgSO₄, filtered, and concentrated. The reside was purified with combi-flash column to afford benzyl (1R,2S)-2-[[(2-tert-butoxy-2-oxo-ethyl)-(chloromethoxycarbonyl)amino]methyl]cyclobutanecarboxylate. MS (m/z) 369.90 [M-isobutene]⁺.

### Step 5: Preparation of benzyl (1R,2S)-2-[[(2-tert-butoxy-2-oxo-ethyl)-(ditert-butoxyphosphoryloxymethoxycarbonyl)amino]methyl]cyclobutanecarboxylate

To a solution of benzyl (1R,2S)-2-[[(2-tert-butoxy-2-oxo-ethyl)-(chloromethoxycarbonyl)amino]methyl]cyclobutanecarboxylate (477 mg, 1.1 mmol) in DME (5 mL) was added ditert-butyl phosphate; tetrabutylammonium (0.71 g, 1.6 mmol) and the reaction was heated to 80° C. for 2h. the reaction was then washed with brine. The organic layer was dried over anhydrous MgSO₄, filtered, and concentrated. The reside was purified with combi-flash column to afford benzyl (1R,2S)-2-[[(2-tert-butoxy-2-oxo-ethyl)-(ditert-butoxyphosphoryloxymethoxycarbonyl)amino]methyl]cyclobutanecarboxylate. MS (m/z) 621.9 [M+Na]⁺.

### Step 6: Preparation of (1R,2S)-2-[[(2-tert-butoxy-2-oxo-ethyl)-(ditert-butoxyphosphoryloxymethoxycarbonyl)amino]methyl]cyclobutanecarboxylic acid

To a solution of benzyl (1R,2S)-2-[[(2-tert-butoxy-2-oxo-ethyl)-(ditert-butoxyphosphoryloxymethoxycarbonyl)amino]methyl]cyclobutanecarboxylate (148 mg, 0.25 mmol) in EtOAc (3 mL) was added 10% Pd/C (0.05 g, 0.049 mmol). The flask was evacuated and backfilled with hydrogen gas (2×), then sparged with hydrogen for 2 min. The reaction mixture was left to stir under a hydrogen balloon atmosphere for 4 h. The reaction mixture was filtered, rinsed with EtOAc, and concentrated to afford (1R,2S)-2-[[(2-tert-butoxy-2-oxo-ethyl)-(ditert-butoxyphosphoryloxymethoxycarbonyl)amino]methyl]cyclobutanecarboxylic acid. MS (m/z) 531.9 [M+Na]⁺.

### Step 7: Preparation of chloromethyl (1R,2S)-2-[[(2-tert-butoxy-2-oxo-ethyl)-(ditert-butoxyphosphoryloxymethoxycarbonyl)amino]methyl]cyclobutanecarboxylate

A biphasic mixture of the (1R,2S)-2-[[(2-tert-butoxy-2-oxo-ethyl)-(ditert-butoxyphosphoryloxymethoxycarbonyl)amino]methyl]cyclobutanecarboxylic acid (0.12 g, 0.24 mmol), tetrabutylammonium hydrogen sulfate (8 mg, 0.02 mmol) and sodium bicarbonate (0.16 g, 1.88 mmol) in water and DCM was cooled to 0° C. While stirring, chloromethyl chlorosulfate (0.078 g, 0.47 mmol) was added dropwise. The reaction mixture was allowed to stir at RT overnight. Brine was added and the aqueous phase was extracted with DCM (3×). The combined organic phase was concentrated. The crude product was purified by column chromatography (0-100% EtOAc/hexanes) to afford chloromethyl (1R,2S)-2-[[(2-tert-butoxy-2-oxo-ethyl)-(ditert-butoxyphosphoryloxymethoxycarbonyl)amino]methyl]cyclobutanecarboxylate. MS (m/z) 579.9 [M+Na]⁺.

### Step 8: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-v)oxy)methyl (1R,2S)-2-(((2-(tert-butoxy)-2-oxoethyl)((((di-tert-butoxyphosphoryl)oxy)methoxy)carbonyl)amino)methyl)cyclobutane-1-carboxylate

Intermediate B (100 mg, 0.21 mmol). K₂CO₃ (57 mg, 0.41 mmol) and KI (44 mg, 0.27 mmol) were mixed in acetone (2 mL) at room temperature. Then a solution of chloromethyl (1R,2S)-2-[[(2-tert-butoxy-2-oxo-ethyl)-(ditert-butoxyphosphoryloxymethoxycarbonyl)amino]methyl]cyclobutanecarboxylate (109 mg, 0.19 mmol) in acetone (1 mL) was added. The reaction mixture was heated at 50 degree for 3 h. The solvent was evaporated and then dissolved in DCM, washed with aqueous saturated NH₄Cl (10 mL). The organic layer was separated and concentrated to dryness. The residue was purified with silica gel column eluted with 80% MeOH/DCM to afford (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (1R,2S)-2-(((2-(tert-butoxy)-2-oxoethyl)((((di-tert-butoxyphosphoryl)oxy)methoxy)carbonyl)amino)methyl)cyclobutane-1-carboxylate. MS (m/z) 1007.8 [M+H]⁺.

### Step 9: Preparation of N-(((1S,2R)-2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)cyclobutyl)methyl)-N-(((phosphonooxy)methoxy)carbonyl)glycine (80)

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 1-(2-((2-(tert-butoxy)-2-oxoethyl)((((di-tert-butoxyphosphoryl)oxy)methoxy)carbonyl)amino)ethyl)cyclopropane-1-carboxylate (51 mg, 0.05 mmol) was dissolved in DCM (2 mL), to the above solution was added TFA (0.3 ml), The reaction mixture was stirred at RT for 5 h. The solvent was evaporated, the residue was purified with C-18 column on Combi-Flash to afford N-(((1S,2R)-2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)cyclobutyl)methyl)-N-(((phosphonooxy)methoxy)carbonyl)glycine. MS (m/z) 839.70 [M+H]⁺. 1H NMR (400 MHz, Methanol-d4) δ 8.57 (d, J=3.4 Hz, 1H), 7.55-7.35 (m, 1H), 7.05-6.85 (m, 2H), 6.00 (dd, J=12.2, 6.3 Hz, 1H), 5.69-5.44 (m, 3H), 4.81 (s, 1H), 4.64 (q, J=15.2 Hz, 2H), 4.50 (d, J=2.6 Hz, 1H), 4.10-3.90 (m, 2H), 3.83 (p, J=1.9 Hz, 2H), 3.60-3.37 (m, 2H), 3.26 (q, J=7.6, 5.3 Hz, 1H), 3.17 (d, J=17.9 Hz, 1H), 2.99 (dq, J=31.4, 7.9 Hz, 1H), 2.71 (dd, J=18.1, 3.5 Hz, 1H), 2.35-2.18 (m, 1H), 2.06 (s, 5H), 2.00-1.77 (m, 5H), 1.60-1.43 (m, 1H), 1.27 (d, J=6.6 Hz, 3H).

### Example 81: Preparation of ((2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-al][1,4]diazonin]-12′-yl)oxy)methoxy)-2-methyl-4-oxobutan-2-yl)-5-methyl-3-(phosphonooxy)phenyl)acetamido)methyl)phosphonic acid (81)

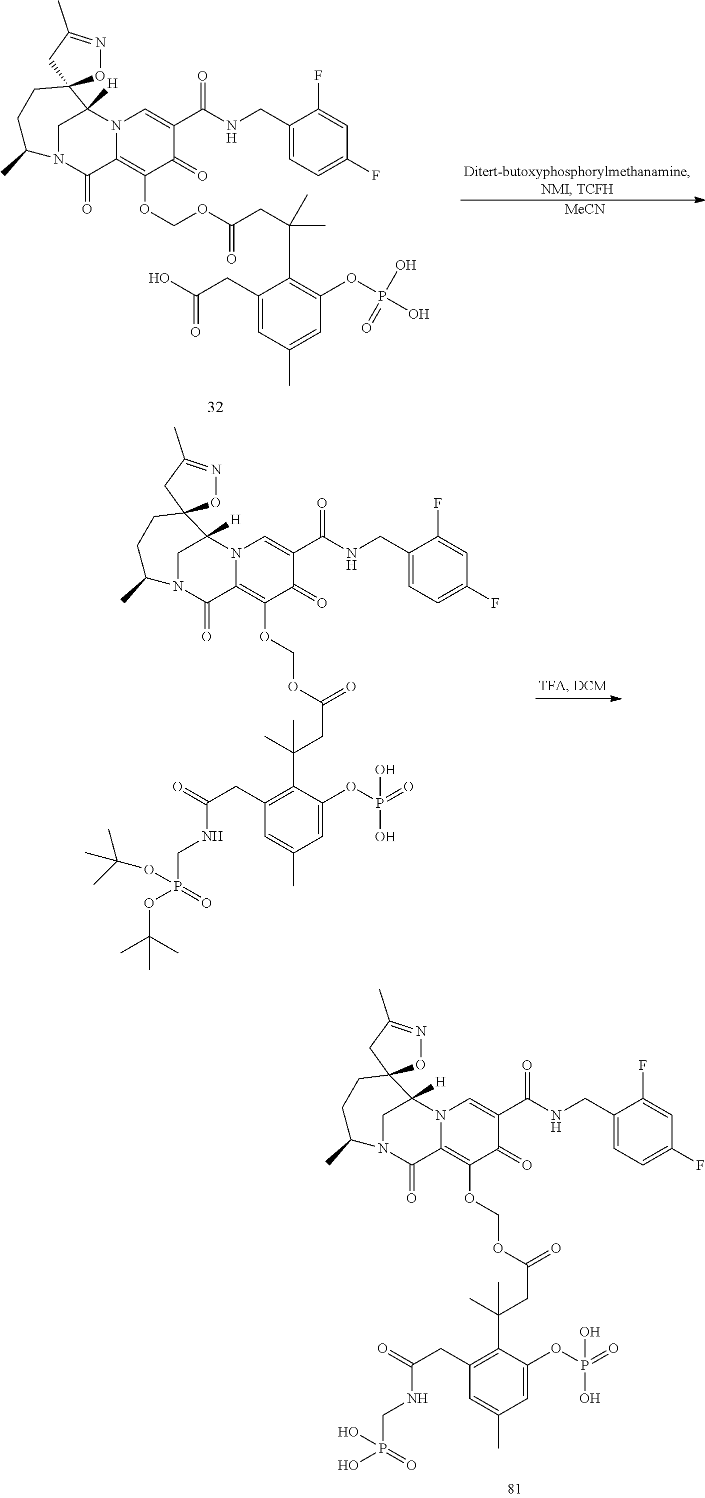 [SMILES: "MeCN" ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)O)cc(C)cc4OP(=O)(O)O)c2C3=O)C1 ; "Ditert-butoxyphosphorylmethanamine,NMI, TCFH" ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)NCP(=O)(OC(C)(C)C)OC(C)(C)C)cc(C)cc4OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)c4c(CC(=O)NCP(=O)(O)O)cc(C)cc4OP(=O)(O)O)c2C3=O)C1]

((2-(2-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2-methyl-4-oxobutan-2-yl)-5-methyl-3-(phosphonooxy)phenyl)acetamido)methyl)phosphonic acid was synthesized following the same procedure as Steps 1-2 of Example 57, except using ditert-butoxy phosphoryl methanamine instead of tert-butyl (2R)-pyrrolidine-2-carboxylate HCl. MS (m/z) 937.6 [M+H]⁺. 1H NMR (400 MHz, Methanol-d4) δ 8.56 (s, 1H), 7.52-7.34 (m, 1H), 7.20 (s, 1H), 7.11-6.87 (m, 2H), 6.76 (d, J=1.9 Hz, 1H), 5.75 (d, J=6.4 Hz, 1H), 5.52 (d, J=6.4 Hz, 1H), 4.81 (q, J=8.1 Hz, 1H), 4.65 (d, J=4.2 Hz, 2H), 4.46 (s, 1H), 3.89 (s, 2H), 3.86-3.70 (m, 2H), 3.70-3.45 (m, 2H), 3.15 (dd, J=17.3, 8.1 Hz, 2H), 3.02 (d, J=17.0 Hz, 1H), 2.69 (d, J=17.9 Hz, 1H), 2.25 (s, 3H), 2.07 (s, 3H), 2.01-1.84 (m, 3H), 1.56 (d, J=5.4 Hz, 7H), 1.26 (d, J=6.7 Hz, 3H).

### Example 82: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl methyl(3-(((((phosphonooxy)methoxy)carbonyl)(3,3,3-trifluoropropyl)amino)methyl)pyridin-2-yl)carbamate (82)

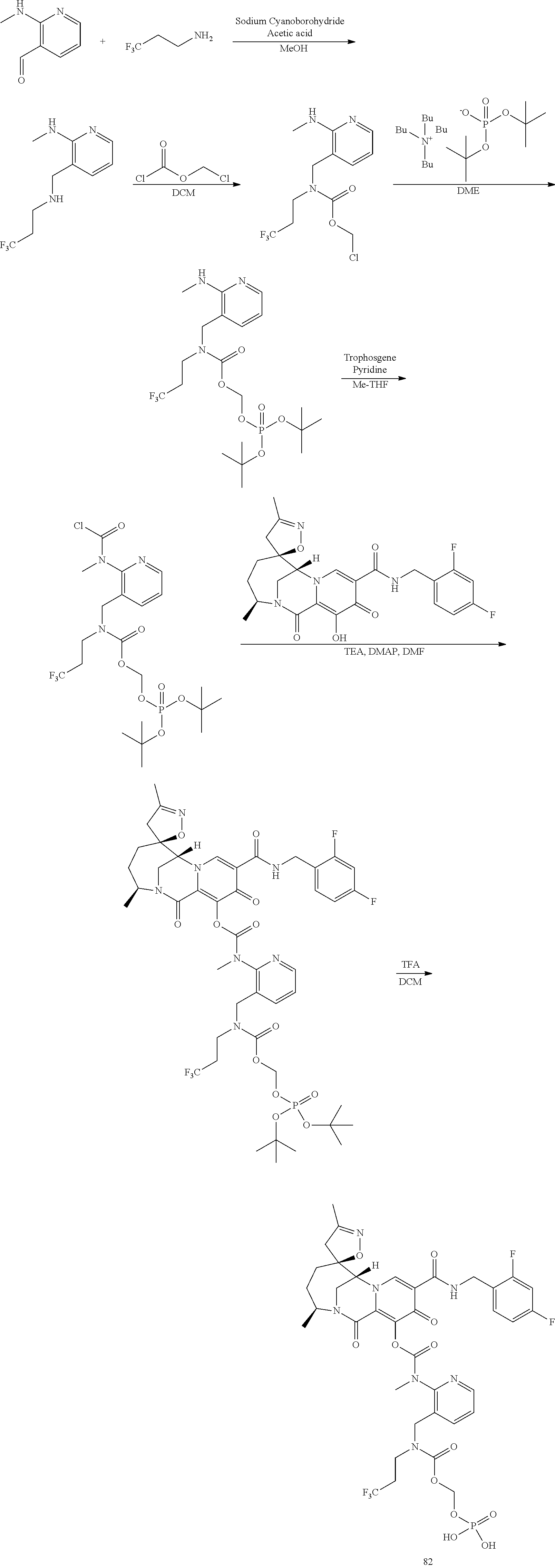

### Step 1: Preparation of N-methyl-3-(propylamino)methyl)pyridin-2-amine

2-(Methylamino)nicotinaldehyde (1.5 g, 11.0 mmol) and 3,3,3-trifluoropropan-1-amine (3.73 g, 33.1 mmol) were dissolved in MeOH (35 mL) at room temperature. Acetic acid (0.806 g, 13.4 mmol) was then added. The reaction mixture was stirred at 40° C. for 90 min and was then cooled down to 0° C. with ice-water bath. To this reaction mixture was added carefully sodium cyanoborohydride (1.39 g, 22 mmol) as solid in one portion. The resulting reaction mixture was then stirred at rt for 90 min. The reaction mixture was then partitioned between EtOAc (10 mL) and saturated NaHCO₃ (10 ml). The organic phase was separated and concentrated. The residue was purified on silica gel column with 0-100% EtOAc/Hex to afford product N-methyl-3-((propylamino)methyl)pyridin-2-amine. MS (m/z): calculated for C₁₀H₁₄F₃N₃, 233.11; found, 234.10 [M+H]⁺.

### Step 2: Preparation of chloromethyl methyl((2-(methylamino)pyridin-3-yl)methyl)carbamate

Chloromethyl ((2-(methylamino)pyridin-3-yl)methyl)(3,3,3-trifluoropropyl)carbamate (530 mg, 2.27 mmol) was dissolved in DCM (7 mL) and the solution was cooled down to −5° C.-0° C. with NaCl-ice bath. Then a solution of chloromethyl chloroformate (272 mg, 2.11 mmol) in DCM (3 ml) was added slowly over 15 min. Let the reaction be stirred at the same temperature for 1 hr. To the reaction mixture was added a saturated aqueous solution of NaHCO₃ (10 mL) at 0° C. The resulting reaction mixture was then stirred at the same temp for 10 min. EtOAc (10 mL) was added. Organic layer was separated and was washed with brine (10 mL). The organic phase was dried over Na₂SO₄ and was filtered. The filtrate was concentrated to dryness to afford product chloromethyl methyl((2-(methylamino)pyridin-3-yl)methyl)carbamate which was used without further purification. MS (m/z): calculated for C₁₂H₁₅ClF₃N₃O₂, 325.08; found, 326.18 [M+H]⁺.

### Step 3: Preparation of ((di-tert-butoxyphosphoryl)oxy)methyl ((2-(methylamino)pyridin-3-yl)methyl)(3,3,3-trifluoropropyl)carbamate

Chloromethyl methyl((2-(methylamino)pyridin-3-yl)methyl)carbamate (720 mg, 2.21 mmol) and tetrabutylammonium di-tert-butyl phosphate (2 g, 4.42 mmol) were mixed with DME (10 mL). The slurry was heated up to 78° C., and kept at that temp with stirring for 1.5 hr. The reaction mixture was cooled down to rt and was partitioned between water (10 mL) and EtOAc (10 mL). The organic phase was separated and washed with brine (10 mL). The organic extraction was concentrated. The residue was purified on silica gel with 0-100% EtOAc/Hex to afford product ((di-tert-butoxyphosphoryl)oxy)methyl ((2-(methylamino)pyridin-3-yl)methyl)(3,3,3-trifluoropropyl)carbamate. MS (m/z): calculated for C₂₀H₃₃F₃N₃O₆P, 499.21; found, 499.98 [M+H]⁺.

### Step 4: Preparation of ((di-tert-butoxyphosphoryl)oxy)methyl ((2-((chlorocarbonyl)(methyl)amino)pyridin-3-yl)methyl)(3,3,3-trifluoropropyl)carbamate

((Di-tert-butoxyphosphoryl)oxy)methyl ((2-(methylamino)pyridin-3-yl)methyl)(3,3,3-trifluoropropyl)carbamate (110 mg, 0.22 mmol) was dissolved in Me-THF (3.3 mL) at room temperature. Under argon balloon, the solution was cooled down to 0° C. Then Pyridine (30 mg, 0.374 mmol) was added with stirring. Ten minutes later, triphosgene (56 mg, 0.18 mmol) was added in one portion. The reaction mixture was stirred at rt overnight and was filtered. The filter cake was washed with Me-THF (10 mL) and EtOAc (10 mL). The filtrate was treated with HCl (1N) (10 mL) and brine (10 mL) and then was dried over Na₂SO₄. The resulting solution was concentrated to afford the product ((di-tert-butoxyphosphoryl)oxy)methyl ((2-((chlorocarbonyl)(methyl)amino)pyridin-3-yl)methyl)(3,3,3-trifluoropropyl)carbamate which was used directly for next step. MS (m/z): calculated for C₂₁H₃₂ClF₃N₃O₇P, 561.16; found, 583.84 [M+Na]⁺.

### Step 5: Preparation of ((di-tert-butoxyphosphoryl)oxy)methyl ((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl) (3,3,3-trifluoropropyl)carbamate

((Di-tert-butoxyphosphoryl)oxy)methyl ((2-((chlorocarbonyl)(methyl)amino)pyridin-3-yl)methyl)(3,3,3-trifluoropropyl)carbamate (129 mg, 0.23 mmol) and (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (70 mg, 0.144 mmol) were dissolved in DMF (5 mL) at rt. Triethylamine (44 mg, 0.432 mmol) and DMAP (11 mg, 0.0863 mmol) were added sequentially. The reaction mixture was stirred at rt for 2 hrs. Reaction mixture was then diluted with EtOAc (10 ml) and was treated with saturated aqueous solution of NH₄Cl (10 mL). Organic phase was separated. The aqueous layer was extracted with EtOAc (1×10 mL). The combined organic phases were washed with water (10 mL) and brine (10 mL) and was concentrated. The residue was purified on silica gel column with 0-100% EtOAc/Hex to afford product ((di-tert-butoxyphosphoryl)oxy)methyl ((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)(3,3,3-trifluoropropyl)carbamate. MS (m/z): calculated for C₄₅H55F₅N₇O₁₂P, 1011.36; found, 1011.62 [M+H]⁺.

### Step 6: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl methyl(3-(((((phosphonooxy)methoxy)carbonyl)(3,3,3-trifluoropropylamino)methyl)pyridin-2-yl)carbamate (82)

((Di-tert-butoxyphosphoryl)oxy)methyl ((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)(3,3,3-trifluoropropyl)carbamate (91 mg, 0.1 mmol) was dissolved in DCM (5 mL) at rt. The solution was cooled down to 0° C. under argon atmosphere. Trifluoroacetic acid (0.5 mL, 6.53 mmol) was added dropwise over 25 min. Reaction was stirred at 0° C. for 70 min. Then TFA and DCM was removed under reduced pressure to afford product (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl methyl(3-(((((phosphonooxy)methoxy)carbonyl)(3,3,3-trifluoropropyl)amino)methyl)pyridin-2-yl)carbamate as a TFA salt. MS (m/z): calculated for C37H39F5N7O12P, 899.23: found, 899.96 [M+H]⁺. 1H NMR (400 MHz, d6-DMSO) δ 10.18 (br, 1H), 8.93-8.66 (m, 1H), 8.46 (s, 1H), 7.92-7.62 (m, 1H), 7.40 (m, 2H), 7.28-7.18 (m, 1H), 7.06 (t, J=8.5 Hz, 1H), 5.68-5.22 (m, 2H), 4.95-4.38 (m, 5H), 3.94-2.51 (m, 12H), 1.94 (s, 3H), 1.83-1.73 (m, 3H), 1.42-1.01 (m, 4H).

### Example 83: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3-(((2-methoxyethyl)(((phosphonooxy)methoxy)carbonyl)amino)methyl)pyridin-2-yl)(methyl)carbamate (83)

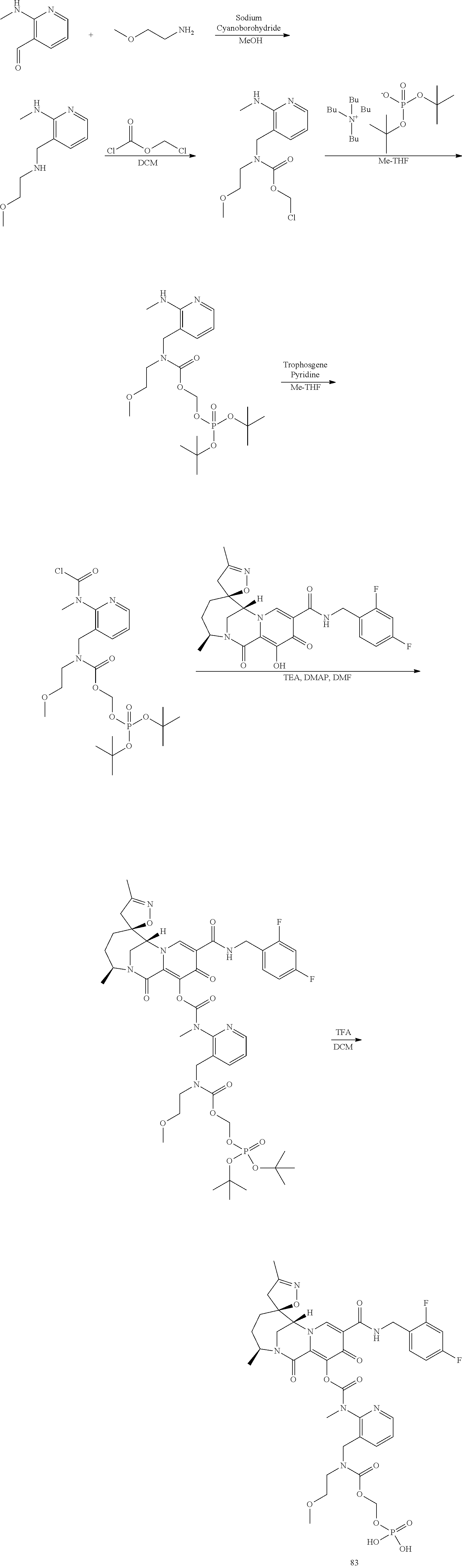

### Step 1: Preparation of 3-((2-methoxyethyl)amino)methyl)-N-methylpyridin-2-amine

2-(Methylamino)nicotinaldehyde (2 g, 14.7 mmol) and 2-methoxyethan-1-amine (3.31 g, 44.1 mmol) were dissolved in MeOH (35 mL) at room temperature. Acetic acid (1.08 g, 17.9 mmol) was then added. The reaction mixture was stirred at 40° C. for 90 min and was then cooled down to 00° C. with ice-water bath. To this reaction mixture was added carefully sodium cyanoborohydride (1.85 g, 29.4 mmol) as solid in one portion. The resulting reaction mixture was then stirred at rt for 90) min. The reaction mixture was then partitioned between EtOAc (10 mL) and saturated NaHCO₃ (10 mL). The organic phase was separated and concentrated. The residue was purified on silica gel column with 0-100% EtOAc/Hex to afford product 3-(((2-methoxyethyl)amino)methyl)-N-methylpyridin-2-amine. MS (m/z): calculated for C10H17N3O, 195.14; found, 196.07 [M+H]⁺.

### Step 2: Preparation of chloromethyl (2-methoxyethyl)((2-(methylamino)pyridin-3-yl)methyl)carbamate

3-(((2-Methoxyethyl)amino)methyl)-N-methylpyridin-2-amine (350 mg, 1.79 mmol) was dissolved in DCM (7 mL) and the solution was cooled down to −5° C.-0° C. with NaCl-ice bath. Then a solution of chloromethyl chloroformate (185 mg, 1.45 mmol) in DCM (3 mL) was added slowly over 15 min. The reaction was stirred at the same temperature for 1 hr. To the reaction mixture was added a saturated aqueous solution of NaHCO₃ (10 mL) at 0° C. The resulting reaction mixture was then stirred at the same temp for 10 min. EtOAc (10 mL) was added. Organic layer was separated and was washed with brine (10 mL). The organic phase was dried over Na₂SO₄ and was filtered. The filtrate was concentrated to dryness to afford product chloromethyl (2-methoxyethyl)((2-(methylamino)pyridin-3-yl)methyl)carbamate which was used without further purification. MS (m/z): calculated for C1₂H₁₈ClN₃O₃, 287.10; found, 288.21 [M+H]⁺.

### Step 3: Preparation of ((di-tert-butoxyphosphoryl)oxy)methyl (2-methoxyethyl)((2-(methylamino)pyridin-3-yl)methyl)carbamate

Chloromethyl (2-methoxyethyl)((2-(methylamino)pyridin-3-yl)methyl)carbamate (516 mg, 1.79 mmol) and tetrabutylammonium di-tert-butyl phosphate (1.62 g, 3.59 mmol) were mixed with Methyl-THF (10 mL). The slurry was heated up to 78° C., and kept at that temp with stirring for 1.5 hr. The reaction mixture was cooled down to rt and was partitioned between water (10 mL) and EtOAc (10 mL). The organic phase was separated and washed with brine (10 mL). The organic extraction was concentrated. The residue was purified on silica gel with 0-100% EtOAc/Hex to afford product ((di-tert-butoxyphosphoryl)oxy)methyl (2-methoxyethyl)((2-(methylamino)pyridin-3-yl)methyl)carbamate. MS (m/z): calculated for C20H36N3O7P, 461.23. found, 462.02 [M+H]+.

### Step 4: Preparation of ((di-tert-butoxyphosphoryl)oxy)methyl ((2-((chlorocarbonyl)(methylamino)pyridin-3-ylmethyl)(2-methoxyethyl)carbamate

((Di-tert-butoxyphosphoryl)oxy)methyl (2-methoxyethyl)((2-(methylamino)pyridin-3-yl)methyl)carbamate (216 mg, 0.468 mmol) was dissolved in Me-THF (5 mL) at room temperature. Under argon balloon, the solution was cooled down to 0° C. Then pyridine (63 mg, 0.796 mmol) was added with stirring. Ten minutes later, triphosgene (119 mg, 0.393 mmol) was added in one portion. The reaction mixture was stirred at rt overnight and was filtered. The filter cake was washed with Me-THF (10 mL) and EtOAc (10 mL). The filtrate was treated with HCl (1N) (10 mL) and brine (10 mL) and then was dried over Na₂SO₄. The resulting solution was concentrated to afford the product ((di-tert-butoxyphosphoryl)oxy)methyl ((2-((chlorocarbonyl)(methyl)amino)pyridin-3-yl)methyl)(2-methoxyethyl)carbamate which was used directly for next step. MS (m/z): calculated for C₂₁H₃₅ClN₃O₈P, 523.19; found, 545.87 [M+Na]⁺.

### Step 5: Preparation of ((di-tert-butoxyphosphoryl)oxy)methyl ((2-(((((3′S,5S,7′R)-10′-((2,4-difluorobenzyl)carbamoyl-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)(2-methoxyethyl)carbamate

((Di-tert-butoxyphosphoryl)oxy)methyl ((2-((chlorocarbonyl)(methyl)amino)pyridin-3-yl)methyl)(2-methoxyethyl)carbamate (207 mg, 0.395 mmol) and (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (120 mg, 0.247 mmol) were dissolved in DMF (5 mL) at rt. Triethylamine (76 mg, 0.76 mmol) and DMAP (19 mg, 0.15 mmol) were added sequentially. The reaction mixture was stirred at rt for 2 hrs. Reaction mixture was then diluted with EtOAc (10 mL) and was treated with saturated aqueous solution of NH₄Cl (10 mL). Organic phase was separated. The aqueous layer was extracted with EtOAc (1×10 mL). The combined organic phases were washed with water (10 mL) and brine (10 mL) and was concentrated. The residue was purified on silica gel column with 0-100% EtOAc/Hex to afford product ((di-tert-butoxyphosphoryl)oxy)methyl ((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)(2-methoxyethyl)carbamate. MS (m/z): calculated for C₄₅H₅₈F₂N₇O₁₃P, 973.38; found, 973.63 [M+H]⁺.

### Step 6: Preparation of (3′S,5S,7′R)-10′-((2,4-difluorobenzyl)carbamoyl)-3,3′-dimeth yl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3-(((2-methoxyethyl)(((phosphonooxy)methoxy)carbonyl)amino)methyl)pyridin-2-yl)(methyl)carbamate (83)

((Di-tert-butoxyphosphoryl)oxy)methyl ((2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)pyridin-3-yl)methyl)(2-methoxyethyl)carbamate (190 mg, 0.195 mmol) was dissolved in DCM (8 mL) at rt. The solution was cooled down to 0° C. under argon atmosphere. Trifluoroacetic acid (0.848 mL, 11.1 mmol) was added dropwise over 25 min. Reaction was stirred at 0° C. for 70 min. Then TFA and DCM was removed under reduced pressure to afford product (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3-(((2-methoxyethyl)(((phosphonooxy)methoxy)carbonyl)amino)methyl)pyridin-2-yl)(methyl)carbamate as a TFA salt. MS (m/z): calculated for C₃₇H₄₂F₂N₇O₁₃P, 861.25: found, 861.80 [M+H]⁺. 1H NMR (400 MHz, d6-DMSO) δ 10.09 (br, 1H), 8.72-7.72 (m, 3H), 7.68-7.45 (m, 1H), 7.41 (q, J=8.4 Hz, 1H), 7.01-6.92 (m, 2H), 5.68-5.36 (m, 2H), 4.87-4.31 (m, 6H), 3.85-3.24 (m, 9H), 3.19 (s, 3H), 3.00 (d, J=17.6 Hz, 1H), 2.59 (d, J=17.6 Hz, 1H), 1.95 (s, 3H), 1.92-1.72 (m, 3H), 1.51-1.29 (m, br, 1H), 1.18 (s, 3H).

### Example 84: Preparation of (6-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)-5-((phosphonooxy)methyl)nicotinoyl)-L-aspartic acid (84)

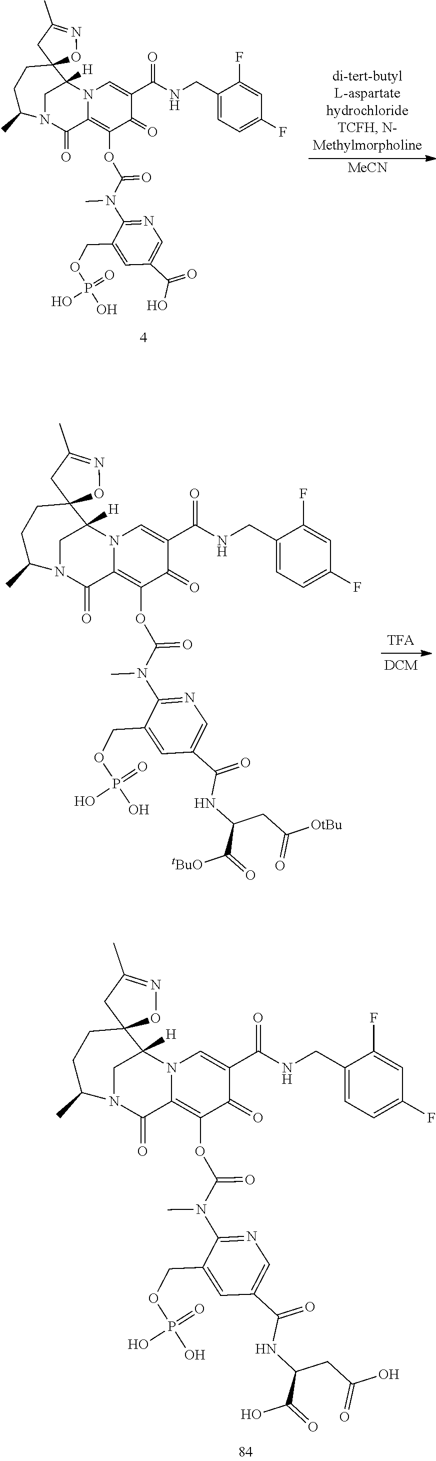 [SMILES: "MeCN" ; CC1=NO[C@@]2(CC[C@H](C)N3C[C@H]2n2cc(C(=O)NCc4ccc(F)cc4F)c(=O)c(OC(=O)N(C)c4ncc(C(=O)O)cc4COP(=O)(O)O)c2C3=O)C1 ; "di-tert-butyl L-aspartate hydrochlorideTCFH, N-Methylmorpholine" ; CC1=NO[C@@]2(CC[C@H](C)N3C[C@H]2n2cc(C(=O)NCc4ccc(F)cc4F)c(=O)c(OC(=O)N(C)c4ncc(C(=O)N[C@@H](CC(=O)OC(C)(C)C)C(=O)OC(C)(C)C)cc4COP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(C)c4ncc(C(=O)N[C@@H](CC(=O)O)C(=O)O)cc4COP(=O)(O)O)c2C3=O)C1]

### Step 1: Preparation of di-tert-butyl (6-(((((3′S,5S,7′R)-10′-((2,4-difluorobenzyl)carbamoyl)-3,3′-dimethyl-1,11′-dioxo-1′,4′,5′,11′-tetrahydro-3′H,4H,7′H-spiro[isoxazole-5,6′-[2,7]methanopyrido[f I2-a][1,4]diazonin]-12′-yl)oxy)carbonyl)(methyl)amino)-5-((phosphonooxy)methyl)nicotinoyl)-L-aspartate

To a mixture of 6-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)-5-((phosphonooxy)methyl)nicotinic acid (Example 4, 139 mg, 0.18 mmol) and di-tert-butyl L-aspartate hydrochloride (76 mg, 0.27 mmol) in ACN (10 mL) at 0° C. was added N-methylmorpholine (55 mg, 0.54 mmol) followed by TCFH (60 mg, 0.21 mmol). The mixture was stirred at 0° C. for 30 minutes and then stirred at room temperature for 30 minutes. Then the reaction mixture was concentrated and the resulting residue was purified by silica gel chromatography to afford di-tert-butyl (6-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)-5-((phosphonooxy)methyl)nicotinoyl)-L-aspartate. MS (m/z) 1002.2 [M+H]⁺.

### Step 2: Preparation of (6-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)-5-((phosphonooxy)methyl)nicotinoyl)-L-aspartic acid

The title compound was made following the same method as Example 23 Step 3, except using di-tert-butyl (6-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)-5-((phosphonooxy)methyl)nicotinoyl)-L-aspartate instead of tert-butyl 2-(3-((di-tert-butoxyphosphoryl)oxy)-2-(4-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylbutan-2-yl)-5-methylphenyl)acetate. MS (m/z) 889.7 [M+H]⁺. 1H NMR (400 MHz, Methanol-d4) δ 8.90 (d, J=2.3 Hz, 1H), 8.64 (d, J=30.9 Hz, 1H), 8.47 (s, 1H), 7.45 (q, J=8.0 Hz, 1H), 7.10-6.83 (m, 2H), 5.00 (dd, J=7.7, 5.2 Hz, 3H), 4.65 (s, 2H), 4.49 (d, J=27.4 Hz, 1H), 3.85 (s, 2H), 3.51 (d, J=60.6 Hz, 3H), 3.14 (d, J=17.7 Hz, 1H), 3.04 (dd, J=16.8, 5.3 Hz, 1H), 2.93 (dd, J=16.7, 7.7 Hz, 1H), 2.69 (d, J=17.9 Hz, 1H), 2.13-1.82 (m, 7H), 1.59 (s, 1H), 1.27 (s, 3H).

### Example 85: Preparation of N-(((3R)-4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)morpholin-3-yl)methyl)-N-(((phosphonooxy)methoxy)carbonyl)glycine (85)

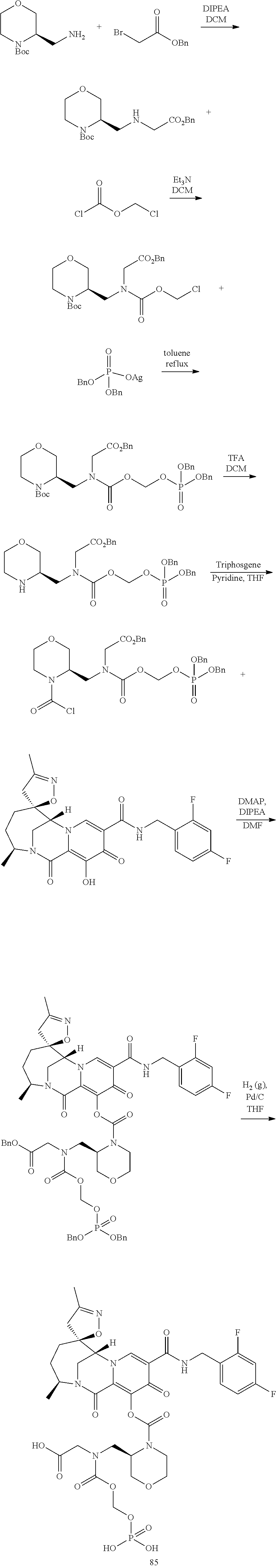

### Step 1: Preparation of tert-butyl (3R)-3-[[(2-benzyloxy-2-oxo-ethyl)amino]methyl]morpholine-4-carboxylate

To a stirred solution of tert-butyl (3R)-3-(aminomethyl)morpholine-4-carboxylate (1.0 g, 4.62 mmol) in DCM (10 mL) at 0° C. under argon was added benzyl 2-bromoacetate (0.53 g, 2.31 mmol) followed by DIPEA (0.89 mL, 5.1 mmol). The reaction mixture was stirred for 16 h at room temperature. The reaction mixture was diluted with DCM and washed with water. The organic layer was dried over anhydrous Na₂SO₄, filtered, and concentrated under reduced pressure. The residue was purified by silica gel column chromatography (30-70% EtOAc/Hexanes) to afford tert-butyl (3R)-3-[[(2-benzyloxy-2-oxo-ethyl)amino]methyl]morpholine-4-carboxylate. MS (m/z) 365.1 [M+H]⁺.

### Step 2: Preparation of tert-butyl (3R)-3-[[(2-benzyloxy-2-oxo-ethyl)-(chloromethoxycarbonyl)amino]methyl]morpholine-4-carboxylate

To a stirred solution of tert-butyl (3R)-3-[[(2-benzyloxy-2-oxo-ethyl)amino]methyl]morpholine-4-carboxylate (0.7 g, 1.92 mmol) in DCM (10 mL) at 0° C. under argon was added chloromethyl chloroformate (0.322 g, 2.5 mmol) followed by triethylamine (0.486 g, 4.8 mmol). The mixture was stirred for 2 h at room temperature. The reaction mixture was diluted with DCM and washed with water. The organic layer was dried over anhydrous Na₂SO₄, filtered, and concentrated under reduced pressure. The crude residue was purified by silica gel column chromatography (20-60% EtOAc/Hexanes) to afford tert-butyl (3R)-3-[[(2-benzyloxy-2-oxo-ethyl)-(chloromethoxycarbonyl)amino]methyl]morpholine-4-carboxylate. MS (m/z) 457.8 [M+H]⁺.

### Step 3: Preparation of tert-butyl (3R)-3-[[(2-benzyloxy-2-oxy-ethyl)-(dibenzyloxyphosphoryloxymethoxycarbonyl)amino]methyl]morpholine-4-carboxylate

To a stirred solution of tert-butyl (3R)-3-[[(2-benzyloxy-2-oxo-ethyl)-(chloromethoxycarbonyl)amino]methyl]morpholine-4-carboxylate (0.8 g, 1.8 mmol) in toluene (10 mL) at room temperature under argon was added silver dibenzylphosphate (1.01 g, 2.6 mmol) under argon. The mixture was stirred at reflux for 16 h. The reaction mixture was allowed to cool to rt and was filtered, rinsing the solids with toluene (5V). The filtrate was concentrated under reduced pressure and purified by silica gel column chromatography (30-70% EtOAc/Hexanes) to afford tert-butyl (3R)-3-[[(2-benzyloxy-2-oxo-ethyl)-(dibenzyloxyphosphoryloxymethoxycarbonyl)amino]methyl]morpholine-4-carboxylate. MS (m/z) 699.1 [M+H]⁺.

### Step 4: Preparation of benzyl 2-[dibenzyloxyphosphoryloxymethoxycarbonyl-[[(3R)-morpholin-3-yl]methyl]amino]acetate

To a stirred solution of tert-butyl (3R)-3-[[(2-benzyloxy-2-oxo-ethyl)-(dibenzyloxyphosphoryloxymethoxycarbonyl)amino]methyl]morpholine-4-carboxylate (1000 mg, 1.43 mmol) in DCM (4 mL) at 0° C. under argon was added 2,2,2-trifluoroacetic acid (1.1 mL) under argon. The mixture was stirred for 2 h at room temperature and concentrated under reduced pressure. The crude residue was washed with NaHCO₃(aq) twice. The organic layer was dried with Na₂SO₄ and filtered. The mixture was concentrated and dried under vacuum to obtain benzyl 2-[dibenzyloxyphosphoryloxymethoxycarbonyl-[[(3R)-morpholin-3-yl]methyl]amino]acetate. MS (m/z) 599.4 [M+H]⁺.

### Step 5: Preparation of benzyl, 2-[[(3R)-4-chlorocarbonylmorpholin-3-yl]methyl-(dibenzyloxyphosphoryloxymethoxycarbonyl)amino]acetate

To a solution of benzyl 2-[dibenzyloxyphosphoryloxymethoxycarbonyl-[[(3R)-morpholin-3-yl]methyl]amino]acetate (240 mg, 0.4 mmol) in THF (2 mL) was added triphosgene (119 mg, 0.4 mmol) at 0° C. Pyridine (0.042 mL, 0.52 mmol) was added to the mixture dropwise. The reaction mixture was warmed to room temperature and stirred for 4 h. It was diluted with EtOAc, washed with IN HCl, water, brine, dried over sodium sulfate, filtered and concentrated to give the title compound, which was used without further purification. MS (m/z) 662.1 [M+H]⁺.

### Step 6: Preparation of (3′S,5S,7′R)-10′-((2,4-difluorobenzyl)carbamoyl-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3R)-3-(((2-(benzyloxy)-2-oxoethyl)((((bis(benzyloxy)phosphoryl)oxy)methoxy)carbonyl)amino)methylImorpholine-4-carboxylate

To a solution of Intermediate B (150 mg, 0.31 mmol) in DMF (5 mL) was added benzyl 2-[[(3R)-4-chlorocarbonylmorpholin-3-yl]methyl-(dibenzyloxyphosphoryloxymethoxycarbonyl)amino]acetate (265 mg, 0.4 mmol) at 0° C. DMAP (22.6 mg, 0.19 mmol) and DIPEA (0.11 mL, 0.62 mmol) were added to the mixture. The reaction mixture was warmed to room temperature and stirred for overnight. It was diluted with EtOAc, washed with IN HCl, water, brine, dried over sodium sulfate, filtered and concentrated which was purified by reverse phase prep HPLC (5-100% MeCN/water) to afford (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3R)-3-(((2-(benzyloxy)-2-oxoethyl)((((bis(benzyloxy)phosphoryl)oxy)methoxy)carbonyl)amino)methyl)morpholine-4-carboxylate. MS (m/z) 1111.7 [M+H]⁺.

### Step 7: Preparation of N-((3RJ-4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)morpholin-3-ylmethyl-N-(((phosphonooxy)methoxy)carbonyl)glycine (85)

To a solution of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3R)-3-(((2-(benzyloxy)-2-oxoethyl)((((bis(benzyloxy)phosphoryl)oxy)methoxy)carbonyl)amino)methyl)morpholine-4-carboxylate (120 mg, 0.11 mmol) in THF (5 mL) was added 10% Pd/C (11.5 mg, 0.011 mmol). The flask was evacuated and backfilled with hydrogen gas (2×), then sparged with hydrogen for 2 min. The reaction mixture was left to stir under a hydrogen balloon atmosphere for 6 h. The reaction mixture was filtered, rinsed with THF, and concentrated to afford a residue, which was purified by reverse phase prep HPLC (5-100% MeCN/water) to afford the title compound. MS (m/z) 841.1 [M+H]⁺. 1H NMR (400 MHz, Methanol-d4) δ 8.66 (d, J=2.7 Hz, 1H), 7.54-7.32 (m, 1H), 7.08-6.86 (m, 2H), 5.89-5.43 (m, 3H), 4.87-4.84 (m, 1H), 4.83-4.31 (m, 5H), 4.32-3.48 (m, 1OH), 3.21-2.98 (m, 1H), 2.70 (d, J=17.8 Hz, 1H), 2.07 (s, 3H), 2.02-1.83 (m, 3H), 1.61 (s, 1H), 1.27 (d, J=6.7 Hz, 3H).

### Example 86: Preparation of N-(((3S)-4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)morpholin-3-yl)methyl)-N-(((phosphonooxy)methoxy)carbonyl)glycine (86)

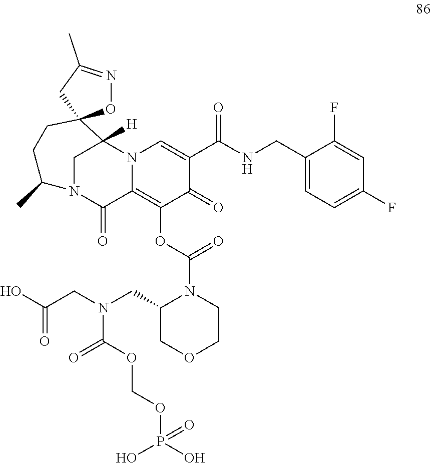 [SMILES: CC1=NO[C@@]2(CC[C@H](C)N3C[C@H]2n2cc(C(=O)NCc4ccc(F)cc4F)c(=O)c(OC(=O)N4CCOC[C@@H]4CN(CC(=O)O)C(=O)OCOP(=O)(O)O)c2C3=O)C1]

N-(((3S)-4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)morpholin-3-yl)methyl)-N-(((phosphonooxy)methoxy)carbonyl)glycine was made following the same method as Example 85, except using tert-butyl (3S)-3-(aminomethyl)morpholine-4-carboxylate instead of tert-butyl (3R)-3-(aminomethyl)morpholine-4-carboxylate. MS (m/z) 841.0 [M+H]⁺. 1H NMR (400 MHz, Methanol-d4) δ 8.66 (s, 1H), 7.46 (q, J=7.9 Hz, 1H), 7.10-6.82 (m, 2H), 5.86-5.47 (m, 3H), 4.78 (d, J=7.7 Hz, 1H), 4.66 (d, J=3.1 Hz, 2H), 4.51 (d, J=10.3 Hz, 1H), 4.40-3.37 (m, 12H), 3.23-2.99 (m, 1H), 2.70 (d, J=17.9 Hz, 1H), 2.07 (s, 3H), 2.03-1.83 (m, 3H), 1.59 (d, J=14.1 Hz, 1H), 1.29 (d, J=6.7 Hz, 3H).

### Example 87: Preparation of N-(((2S)-1-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)-4-methylpiperazin-2-yl)methyl)-N-(((phosphonooxy)methoxy)carbonyl)glycine (87)

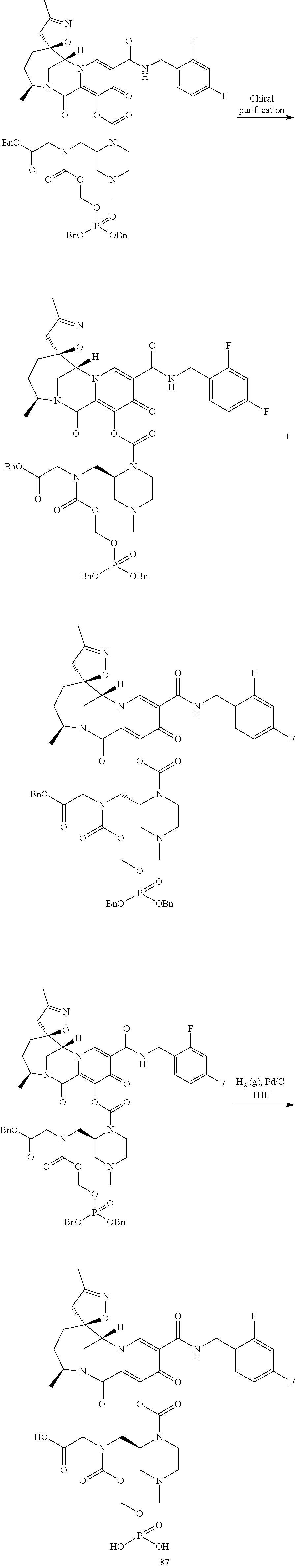

### Step 1: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2S)-2-(((2-(benzyloxy)-2-oxoethyl)((((bis(benzyloxy)phosphoryl)oxy)methoxy)carbonyl)amino)methyl)-4-methylpiperazine-1-carboxylate and (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2R)-2-(((2-(benzyloxy)-2-oxoethyl)((((bis(benzyloxy)phosphoryl)oxy)methoxy)carbonyl)amino)methyl-4-methylpiperazine-1-carboxylate

(3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-(((2-(benzyloxy)-2-oxoethyl)((((bis(benzyloxy)phosphoryl)oxy)methoxy)carbonyl)amino)methyl)-4-methylpiperazine-1-carboxylate, prepared according to the same method as Example 85 except using tert-butyl 2-(aminomethyl)-4-methyl-piperazine-1-carboxylate instead of tert-butyl (3R)-3-(aminomethyl)morpholine-4-carboxylate, was purified by chiral purification to afford the two title products. Peak 1. MS (m/z) 1124.1 [M+H]. Peak 2: MS (m/z) 1124.0 [M+H]⁺.

### Step 2: Preparation of N-(((2S)-1-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxycarbonyl)-4-methylpiperazin-2-yl)methyl)-N-(((phosphonooxy)methoxy)carbonyl)glycine (87)

To a solution of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2S)-2-(((2-(benzyloxy)-2-oxoethyl)((((bis(benzyloxy)phosphoryl)oxy)methoxy)carbonyl)amino)methyl)-4-methylpiperazine-1-carboxylate (15 mg, 0.13 mmol) in 3 mL of THF was added 7.1 mg of 10% palladium on carbon. The flask was evacuated and backfilled with hydrogen gas (2×), then sparged with hydrogen for 2 min. The reaction mixture was left to stir under a hydrogen balloon atmosphere for 2 h. The reaction mixture was filtered, rinsed with THF, and concentrated to afford a residue, which was purified by reverse phase prep HPLC (5-100% MeCN/water) to afford N-(((2S)-1-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)-4-methylpiperazin-2-yl)methyl)-N-(((phosphonooxy)methoxy)carbonyl)glycine. MS (m/z) 854.0 [M+H]⁺. 1H NMR (400 MHz, Methanol-d4) δ 10.38 (s, 1H), 8.69 (s, 1H), 7.46 (q, J=7.9 Hz, 1H), 7.22-6.81 (m, 2H), 5.67 (s, 2H), 4.90 (s, 1H), 4.83-4.48 (m, 6H), 4.48-3.45 (m, 8H), 3.22-3.06 (m, 2H), 3.01 (s, 3H), 2.71 (d, J=17.8 Hz, 1H), 2.07 (s, 3H), 2.02-1.82 (m, 3H), 1.56 (s, 1H), 1.28 (d, J=6.6 Hz, 3H).

### Example 88: Preparation of N-(((2R)-1-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)-4-methylpiperazin-2-yl)methyl)-N-(((phosphonooxy)methoxy)carbonyl)glycine (88)

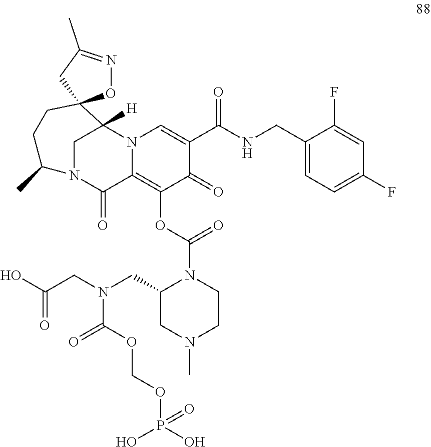 [SMILES: CC1=NO[C@@]2(CC[C@H](C)N3C[C@H]2n2cc(C(=O)NCc4ccc(F)cc4F)c(=O)c(OC(=O)N4CCN(C)C[C@@H]4CN(CC(=O)O)C(=O)OCOP(=O)(O)O)c2C3=O)C1]

N-(((2R)-1-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)-4-methylpiperazin-2-yl)methyl)-N-(((phosphonooxy)methoxy)carbonyl)glycine was prepared in a manner similar to Example 87, except using (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2R)-2-(((2-(benzyloxy)-2-oxoethyl)((((bis(benzyloxy)phosphoryl)oxy)methoxy)carbonyl)amino)methyl)-4-methylpiperazine-1-carboxylate instead of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2S)-2-(((2-(benzyloxy)-2-oxoethyl)((((bis(benzyloxy)phosphoryl)oxy)methoxy)carbonyl)amino)methyl)-4-methylpiperazine-1-carboxylate in Step 2. MS (m/z) 854.0 [M+H]⁺. 1H NMR (400 MHz, Methanol-d4) δ 10.37 (s, 1H), 8.70 (d, J=3.6 Hz, 1H), 7.54-7.37 (m, 1H), 7.09-6.85 (m, 2H), 5.68 (dt, J=37.3, 20.2 Hz, 2H), 4.84-4.41 (m, 6H), 4.44-4.04 (m, 3H), 4.05-3.69 (m, 4H), 3.71-3.46 (m, 2H), 3.37 (s, 2H), 3.19-3.07 (m, 2H), 3.01 (s, 3H), 2.70 (d, J=18.1 Hz, 1H), 2.00-1.82 (m, 3H), 1.57 (s, 1H), 1.26 (d, J=6.6 Hz, 3H).

### Example 89: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (tetrahydro-2H-pyran-4-yl) carbonate (89)

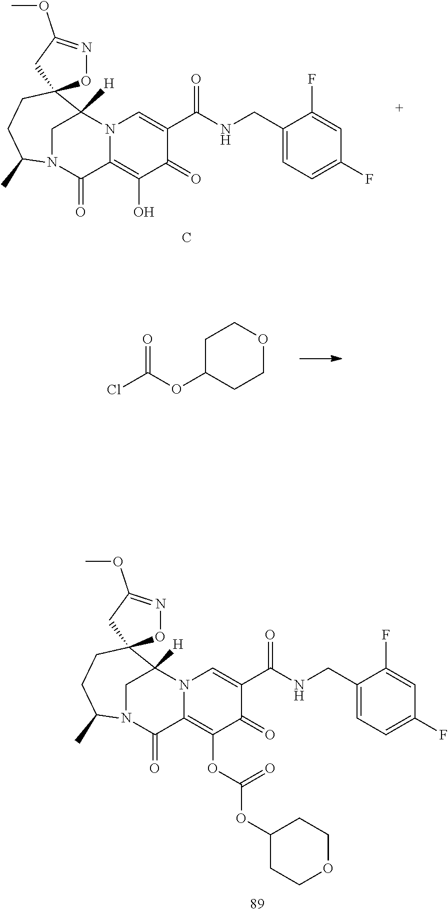

To a mixture of Intermediate C (0.2 g, 0.398 mmol) in a mixture of DCM (2.0 mL) and DMF (2.0 mL) at room temperature was added tetrahydro-2H-pyran-4-yl carbonochloridate (98.3 mg, 0.597 mmol). The resulting mixture was stirred for 16 hours before it was diluted with DCM, washed sequentially with water, 5% LiCl and brine. Then it was dried over sodium sulfate, filtered, concentrated and purified by reverse phase prep HPLC, eluted with 0-90% ACN/H2O with 0.1% TFA) to give title compound. MS (m/z) 631.2 [M+H]+. 1H NMR (400 MHz, Chloroform-d) δ 10.38 (t, J=5.9 Hz, 1H), 8.83 (s, 1H), 7.35 (td, J=8.4, 6.3 Hz, 1H), 6.91-6.76 (m, 2H), 4.97 (tt, J=8.1, 4.0 Hz, 1H), 4.89-4.76 (m, 1H), 4.70 (dd, J=15.0, 6.2 Hz, 1H), 4.57 (dd, J=15.1, 5.6 Hz, 1H), 4.45 (s, 1H), 4.00 (ddd, J=11.7, 6.0, 4.0 Hz, 2H), 3.86 (s, 3H), 3.81 (dd, J=15.2, 1.8 Hz, 1H), 3.69 (dd, J=15.2, 2.7 Hz, 1H), 3.61 (ddd, J=11.7, 8.3, 3.3 Hz, 2H), 3.10 (d, J=17.1 Hz, 1H), 2.66 (d, J=17.1 Hz, 1H), 2.16-1.83 (m, 7H), 1.67-1.55 (m, 1H), 1.26 (d, J=6.7 Hz, 3H).

### Example 90: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (tetrahydro-2H-pyran-4-yl) carbonate (90)

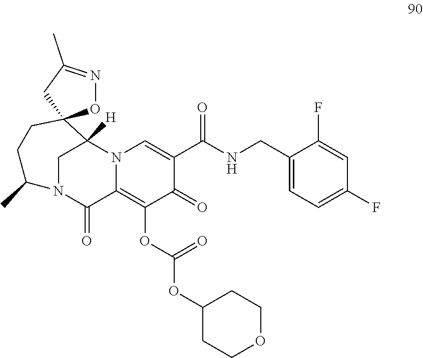 [SMILES: CC1=NO[C@@]2(CC[C@H](C)N3C[C@H]2n2cc(C(=O)NCc4ccc(F)cc4F)c(=O)c(OC(=O)OC4CCOCC4)c2C3=O)C1]

The title compound was made in a manner similar to Example 89 except Intermediate B was used instead of Intermediate C. MS (m/z) 615.21 [M+H]+. 1H NMR (400 MHz, Chloroform-d) δ 10.33 (t, J=6.0 Hz, 1H), 8.54 (s, 1H), 7.35 (td, J=8.7, 6.4 Hz, 1H), 6.92-6.76 (m, 2H), 4.98 (tt, J=8.1, 4.0 Hz, 1H), 4.82 (dq, J=10.1, 6.8 Hz, 1H), 4.68 (dd, J=15.0, 6.1 Hz, 1H), 4.59 (dd, J=15.1, 5.8 Hz, 1H), 4.17 (d, J=2.1 Hz, 1H), 3.99 (ddd, J=11.8, 5.9, 4.0 Hz, 2H), 3.82 (dd, J=15.2, 1.9 Hz, 1H), 3.69 (dd, J=15.2, 2.7 Hz, 1H), 3.60 (ddd, J=11.7, 8.3, 3.3 Hz, 2H), 3.08 (d, J=17.8 Hz, 1H), 2.66-2.56 (m, 1H), 2.16-2.02 (m, 5H), 2.01-1.83 (m, 5H), 1.67-1.53 (m, 1H), 1.26 (d, J=6.7 Hz, 3H).

### Example 91: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-((tetradecanoyloxy)methyl)benzoate (91)

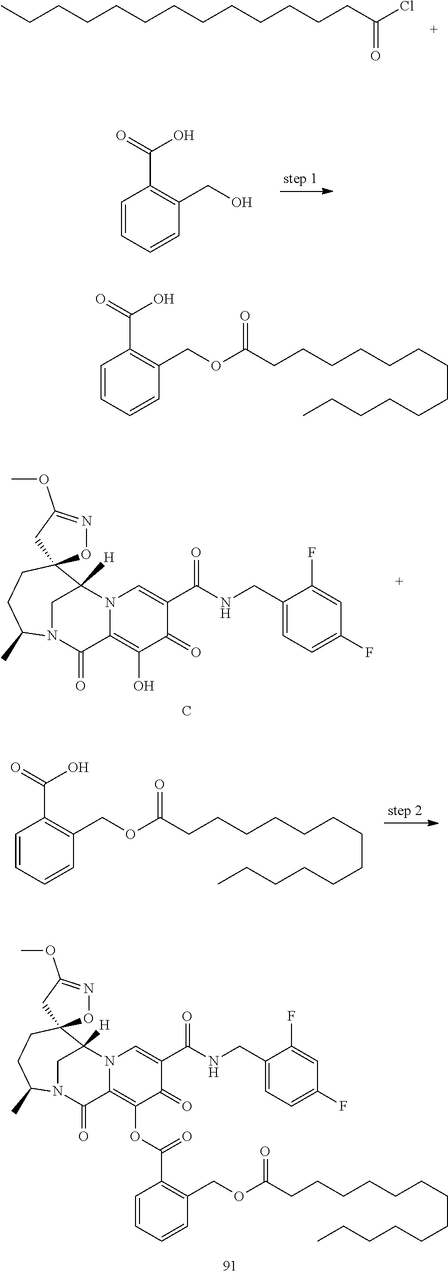

### Step 1: Synthesis of 2-((tetradecanoyloxy)methyl)benzoic acid

The mixture of 2-(hydroxymethyl)benzoic acid (2.0 g, 13.1 mmol) in 20% aqueous NaOH (12 mL) was heated at 35° C. for 30 min before it was cooled to 0° C. To this vigorously stirred mixture was added tetradecanoyl chloride (3.828 g, 15.5 mmol) quickly. The newly formed mixture was stirred 15 min. then it was filtered. The filter cake was rinsed with water and then transferred into a flask. The solid was suspended with water (15 mL) and acidified to pH ˜2 with IN HCL. The suspension was filtered and the filter cake was rinsed with water. The solid was then mixed with DCM and silica gel, concentrated to dryness, loaded into a cartridge, and purified by silica gel column chromatography, eluted with 0-50% EtOAc/Hexane to give title compound. 1H NMR (400 MHz, Chloroform-d) δ 8.15 (dd, J=7.8, 1.3 Hz, 1H), 7.64-7.58 (m, 1H), 7.55 (d, J=7.5 Hz, 1H), 7.47-7.40 (m, 1H), 5.60 (s, 2H), 2.43 (t, J=7.6 Hz, 2H), 1.77-1.63 (m, 2H), 1.33-1.24 (m, 21H), 0.89 (t, J=6.7 Hz, 3H).

### Step 2: Synthesis of (3′S,5S,7′RJ-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-((tetradecanoyloxy)methyl)benzoate (91)

To a mixture of Intermediate C (0.16 g, 0.318 mmol) and 2-((tetradecanoyloxy)methyl)benzoic acid (173 mg, 0.478 mmol) in DMF (5.0 mL) at room temperature was added 1-(3-dimethylaminopropyl)-3-ethylcarbodiimide hydrochloride (91.6 mg, 0.478 mmol) followed by DMAP (11.7 mg, 0.096 mmol) and DIEA (103 mg, 0.796 mmol). The resulting mixture was heated at 38° C. for 16 hours. The reaction was then diluted with EtOAc, washed with 5% aq LiCl, brine, dried over sodium sulfate, filtered and concentrated, purified by silica gel column chromatography, eluted with 0-100% EtOAc/Hexane, to provide the title compound. MS (m/z) 847.39 [M+H]+; 1H NMR (400 MHz, Chloroform-d) δ 10.27 (t, J=5.8 Hz, 1H), 8.94 (d, J=66.8 Hz, 1H), 8.49-8.14 (m, 1H), 7.66-7.53 (m, 2H), 7.48-7.31 (m, 2H), 6.89-6.73 (m, 2H), 5.60 (s, 2H), 4.86-4.43 (m, 4H), 3.88-3.68 (m, 5H), 3.15 (d, J=17.0 Hz, 1H), 2.63 (d, J=17.0 Hz, 1H), 2.43 (t, J=7.6 Hz, 2H), 2.04-1.85 (m, 3H), 1.74-1.58 (m, 3H), 1.27 (s, 20H), 1.24-1.19 (m, 3H), 0.89 (t, J=6.8 Hz, 3H).

### Example 92: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-((tetradecanoyloxy)methyl)benzoate (92)

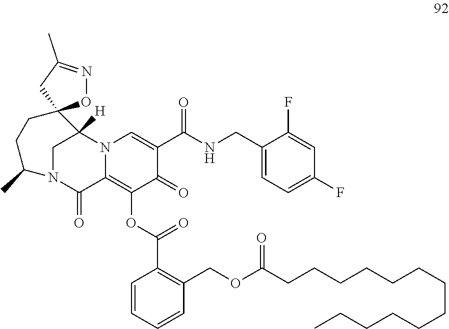 [SMILES: CCCCCCCCCCCCCC(=O)OCc1ccccc1C(=O)Oc1c2n(cc(C(=O)NCc3ccc(F)cc3F)c1=O)[C@@H]1CN(C2=O)[C@@H](C)CC[C@]12CC(C)=NO2]

The title compound was made in a manner similar to Example 91 except Intermediate B was used instead of Intermediate C. MS (m/z) 831.39 [M+H]+. 1H NMR (400 MHz, Chloroform-d) δ 10.26-10.12 (m, 1H), 8.48 (s, 1H), 8.41-8.17 (m, 1H), 7.66-7.55 (m, 2H), 7.49-7.40 (m, 1H), 7.36 (td, J=8.4, 6.3 Hz, 1H), 6.89-6.70 (m, 2H), 5.60 (s, 2H), 4.85-4.51 (m, 3H), 4.15 (s, 1H), 3.86-3.67 (m, 2H), 3.08 (d, J=17.8 Hz, 1H), 2.71-2.55 (m, 1H), 2.43 (t, J=7.6 Hz, 2H), 2.11 (s, 3H), 2.00-1.86 (m, 3H), 1.71-1.56 (m, 3H), 1.27 (s, 20H), 1.25-1.20 (m, 3H), 0.89 (t, J=6.8 Hz, 3H).

### Example 93: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-(4-tetradecanamidophenyl)acetate (93)

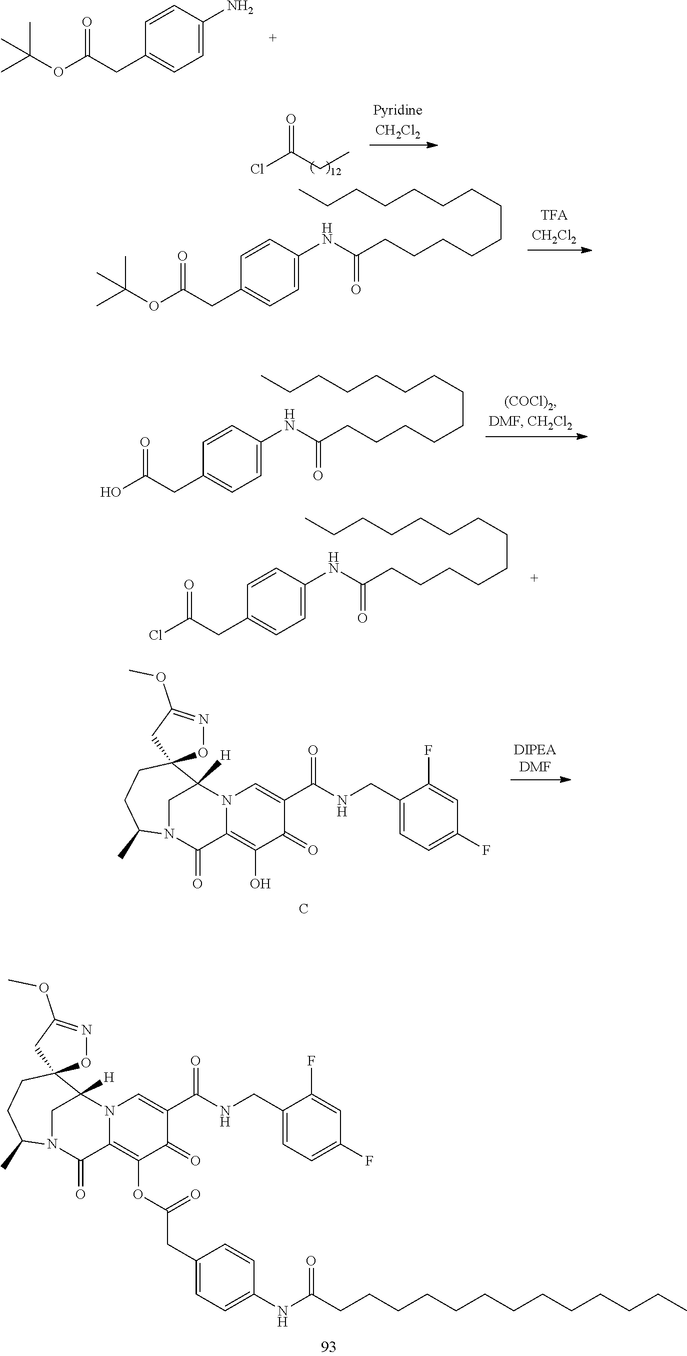

### Step 1: Synthesis of tert-butyl 2-(4-tetradecanamidophenyl)acetate

To a solution of tert-butyl 2-(4-aminphenyl)acetate (0.500 g, 2.41 mmol) in CH₂Cl₂ (5 mL) was added pyridine (0.291 mL, 3.62 mmol) and myristoyl chloride (0.721 mL, 2.65 mmol). The reaction mixture was stirred at rt for 6 h and quenched with water and 1 N HCl. The phases were separated, and the aqueous phase was extracted with CH₂Cl₂. The combined organic phase was washed with brine, dried over Na₂SO₄, filtered, and concentrated to afford the title compound. MS (m/z) 418.33 [M+H]+.

### Step 2: Synthesis of 2-(4-tetradecanamidophenyl)acetic acid

To a mixture of tert-butyl 2-(4-tetradecanamidophenyl)acetate (1.01 g, 2.42 mmol) in CH₂Cl₂ (20 mL) was added TFA (2.77 mL). The reaction mixture was stirred at rt overnight, concentrated, and used directly in the next step without further purification. MS (m/z) 362.28 [M+H]+.

### Step 3: Synthesis of 2-(4-tetradecanamidophenyl)acetyl chloride

To a solution of 2-(4-tetradecanamidophenyl)acetic acid (0.210 g, 0.581 mmol) in CH₂Cl₂ (3 mL) was added oxalyl chloride (0.123 mL, 1.45 mmol) followed by 1 drop of DMF. The reaction mixture was stirred at rt overnight, concentrated, and used directly in the next step without further purification.

### Step 4: Synthesis of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-(4-tetradecanamidophenyl)acetate (93)

To a vial containing Intermediate C (0.100 g, 0.199 mmol) was added pyridine (1.5 mL). The mixture was stirred briefly and concentrated to dryness. The residue was suspended in DMF (1.0 mL) and DIPEA (0.069 mL, 0.398 mmol) was added. A solution of 2-(4-tetradecanamidophenyl)acetyl chloride (0.108 g, 0.299 mmol) in DMF (0.5 mL) was added and the reaction mixture was left to stir at rt overnight. The reaction mixture was quenched with 5% aqueous LiCl, diluted with EtOAc, and the phases were separated. The organic phase was washed with water and brine, dried over Na₂SO₄, filtered, and concentrated. The residue was purified by silica gel column chromatography (0-100% EtOAc/hexane) to afford the title product. MS (m/z) 845.79 [M+H]+. 1H NMR (400 MHz, Chloroform-d) δ 10.16 (s, 1H), 8.48 (s, 1H), 7.52-7.41 (m, 2H), 7.34 (d, J=7.9 Hz, 2H), 7.31 (s, 1H), 6.81 (q, J=9.8, 9.1 Hz, 2H), 4.80 (q, J=7.8 Hz, 1H), 4.70-4.61 (m, 1H), 4.56 (d, J=14.9 Hz, 1H), 4.19 (s, 1H), 3.95 (d, J=9.1 Hz, 2H), 3.89 (s, 3H), 3.77 (d, J=15.2 Hz, 1H), 3.64 (d, J=14.7 Hz, 1H), 3.04 (d, J=17.1 Hz, 1H), 2.66 (d, J=17.5 Hz, 1H), 2.37 (dt, J=30.6, 7.5 Hz, 2H), 1.97 (dt, J=26.5, 10.5 Hz, 3H), 1.72 (q, J=7.4 Hz, 3H), 1.25 (s, 24H), 0.88 (t, J=6.8 Hz, 3H).

### Example 94: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-(4-tetradecanamidophenyl)acetate (94)

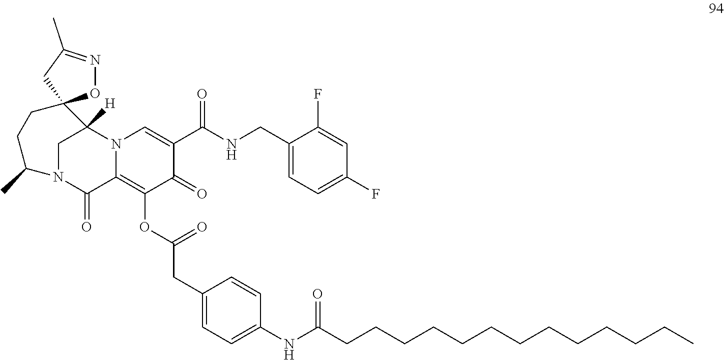 [SMILES: CCCCCCCCCCCCCC(=O)Nc1ccc(CC(=O)Oc2c3n(cc(C(=O)NCc4ccc(F)cc4F)c2=O)[C@@H]2CN(C3=O)[C@@H](C)CC[C@]23CC(C)=NO3)cc1]

The title compound was prepared in a similar manner to Example 93, except using Intermediate B instead of Intermediate C in Step 4. MS (m/z) 829.92 [M+H]+. 1H NMR (400 MHz, Chloroform-d) δ 10.16 (s, 1H), 8.39 (s, 1H), 7.64-7.42 (m, 2H), 7.32 (dd, J=17.8, 8.6 Hz, 3H), 6.81 (q, J=9.4, 8.7 Hz, 2H), 4.80 (d, J=7.4 Hz, 1H), 4.65 (d, J=15.4 Hz, 1H), 4.56 (d, J=15.8 Hz, 1H), 4.06 (d, J=9.6 Hz, 1H), 3.95 (d, J=9.1 Hz, 2H), 3.79 (d, J=15.1 Hz, 1H), 3.66 (d, J=15.1 Hz, 1H), 3.07-2.97 (m, 1H), 2.58 (d, J=17.8 Hz, 1H), 2.37 (dt, J=30.4, 7.6 Hz, 2H), 2.09 (s, 3H), 2.01-1.80 (m, 3H), 1.72 (q, J=7.3 Hz, 2H), 1.26 (s, 25H), 0.90-0.86 (m, 3H).

### Example 95: [4-[2-[(1R,10S,13S)-4-[(2,4-difluorophenyl)methylcarbamoyl]-3′-methoxy-10-methyl-5,8-dioxo-spiro[2,9-diazatricyclo[7.4.1.02,7]tetradeca-3,6-diene-13,5′-4H-isoxazole]-6-yl]oxy-2-oxo-ethyl]phenyl] tetradecanoate (95)

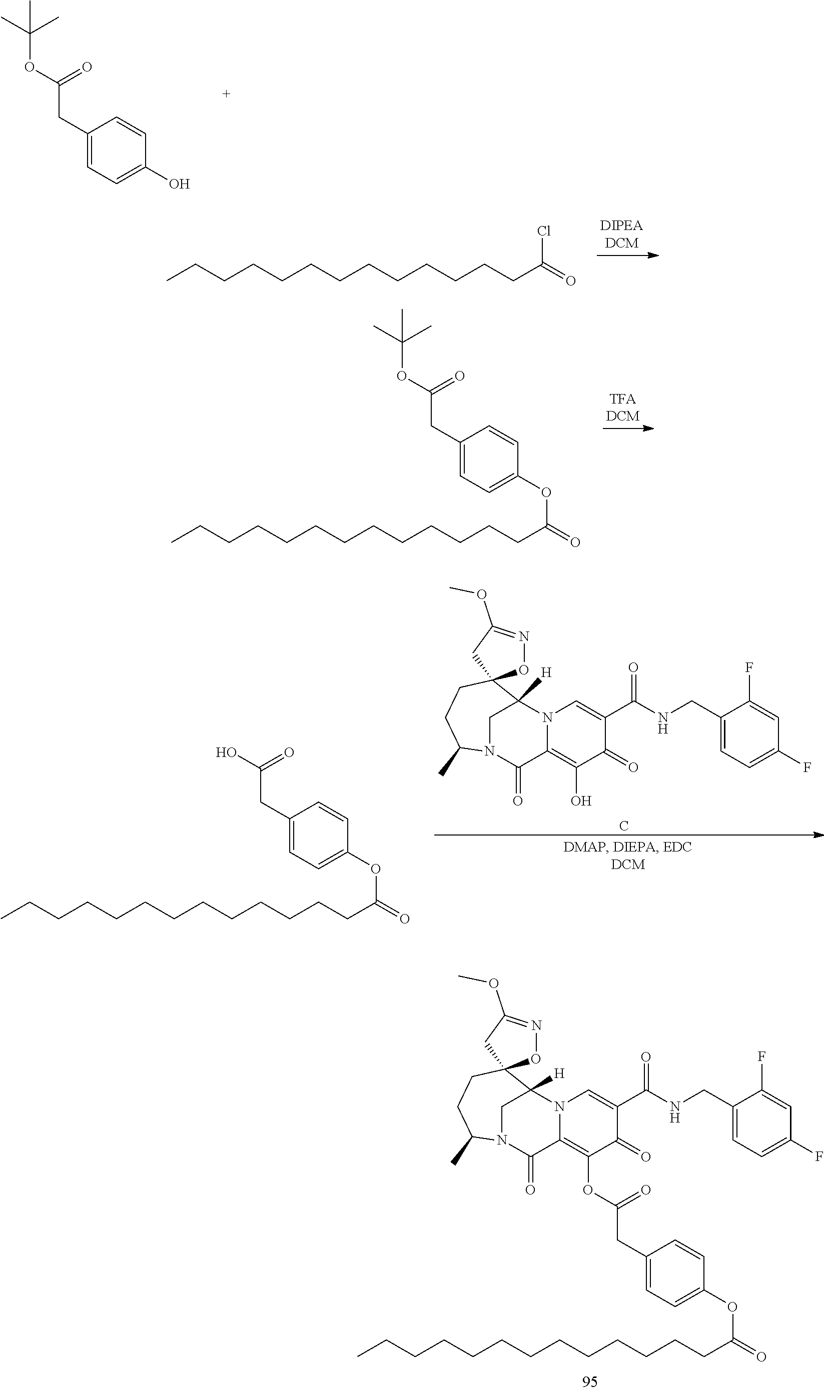

### Step 1: Preparation of [4-(2-tert-butoxy-2-oxo-ethyl)phenyl] tetradecanoate

To a stirred solution of tert-butyl 2-(4-hydroxyphenyl)acetate (1.1 g, 5.28 mmol) in DCM (10 mL) at 0° C. under argon was added tetradecanoyl chloride (1.96 g, 7.92 mmol) followed by DIPEA (2.3 mL, 13.2 mmol). The reaction mixture was stirred for 16 h at room temperature. The reaction mixture was diluted with DCM and washed with water. The organic layer was dried over anhydrous Na₂SO₄ filtered, and concentrated under reduced pressure. The residue was purified by silica gel column chromatography (10-70% EtOAc/Hexanes) to afford the title compound. MS (m/z) 419.1 [M+H]+.

### Step 2: Preparation of 2-(4-tetradecanoyloxyphenyl)acetic acid

To a stirred solution of [4-(2-tert-butoxy-2-oxo-ethyl)phenyl]tetradecanoate (0.45 g, 1.07 mmol) in DCM (2 mL) at 0° C. under argon was added TFA (0.5 mL). The mixture was stirred for 2 h at room temperature. The reaction mixture was concentrated. Used without further purification. MS (m/z) 363.2 [M+H]+.

### Step 3: Preparation of [4-[2-[(1R,10S,13S)-4-[(2,4-difluorophenyl)methylcarbamoyl]-3′-methoxy-10-methyl-5,8-dioxo-spiro[2,9-diazatricyclo[7.4.1.02,7]tetradeca-3,6-diene-13,5′-4H-isoxazole]-6-yl]oxy-2-oxo-ethyl]phenyl] tetradecanoate (95)

To a solution of Intermediate C (200 mg, 0.4 mmol) and 2-(4-tetradecanoyloxyphenyl)acetic acid (173 mg, 0.478 mmol) in DCM (3 mL) was added 3-(ethyliminomethyleneamino)-N,N-dimethyl-propan-1-amine; hydrochloride (114 mg, 0.6 mmol), DIEPA (0.14 mL, 0.8 mmol), and 4-Dimethylaminopyridine (48.6 mg, 0.4 mmol). The reaction mixture was stirred for 16 hours. The reaction mixture was filtered, rinsed with THF, and concentrated to afford a residue, which was purified by silica gel column chromatography (10-70% EtOAc/Hexanes) to afford the title compound. MS (m/z) 848.5 [M+H]+. 1H NMR (400 MHz, Chloroform-d) δ 10.27 (t, J=5.9 Hz, 1H), 8.98 (s, 1H), 7.42 (d, J=8.4 Hz, 2H), 7.37 (dd, J=8.4, 6.4 Hz, 1H), 7.14-7.00 (m, 2H), 6.83 (dtd, J=11.8, 8.6, 2.6 Hz, 2H), 4.82 (dt, J=10.4, 6.7 Hz, 1H), 4.73 (dd, J=15.2, 6.2 Hz, 1H), 4.62 (s, 1H), 4.52 (dd, J=15.3, 5.3 Hz, 1H), 4.01 (s, 2H), 3.83-3.60 (m, 6H), 3.10 (d, J=17.0 Hz, 1H), 2.64-2.48 (m, 3H), 2.09-1.84 (m, 3H), 1.75 (p, J=7.5 Hz, 2H), 1.68-1.50 (m, 1H), 1.50-1.17 (m, 22H), 0.97-0.81 (m, 3H).

### Example 96: [4-[2-[(1R,10S,13S)-4-[(2,4-difluorophenyl)methylcarbamoyl]-3′,10-dimethyl-5,8-dioxo-spiro[2,9-diazatricyclo[7.4.1.02,7]tetradeca-3,6-diene-13,5′-4H-isoxazole]-6-yl]oxy-2-oxo-ethyl]phenyl] tetradecanoate (96)

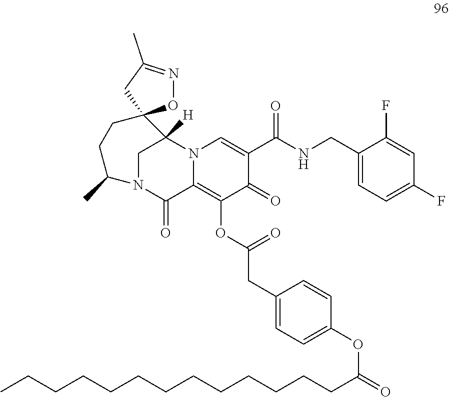 [SMILES: CCCCCCCCCCCCCC(=O)Oc1ccc(CC(=O)Oc2c3n(cc(C(=O)NCc4ccc(F)cc4F)c2=O)[C@@H]2CN(C3=O)[C@@H](C)CC[C@]23CC(C)=NO3)cc1]

The title compound was made following the same method as Example 95, except using Intermediate B instead of Intermediate C in Step 3. MS (m/z) 832.6 [M+H]+. 1H NMR (400 MHz, Chloroform-d) δ 10.19 (t, J=6.1 Hz, 1H), 8.45 (s, 1H), 7.42 (d, J=8.4 Hz, 2H), 7.35 (td, J=8.7, 6.5 Hz, 1H), 7.16-6.98 (m, 2H), 6.97-6.75 (m, 2H), 4.80 (dq, J=9.4, 6.8 Hz, 1H), 4.68 (dd, J=15.1, 6.0 Hz, 1H), 4.57 (dd, J=15.2, 5.6 Hz, 1H), 4. It (d, J=2.2 Hz, 1H), 4.01 (s, 2H), 3.78 (dd, J=15.2, 1.9 Hz, 1H), 3.67 (dd, J=15.1, 2.7 Hz, 1H), 3.04 (d, J=17.7 Hz, 1H), 2.68-2.49 (m, 3H), 2.08 (s, 3H), 2.03-1.86 (m, 3H), 1.75 (p, J=7.5 Hz, 2H), 1.59 (ddd, J=15.1, 8.6, 4.1 Hz, 1H), 1.50-1.14 (m, 23H), 0.90 (t, J=6.7 Hz, 3H).

### Example 97: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 4-((tetradecanoyloxy)methyl)benzoate (97)

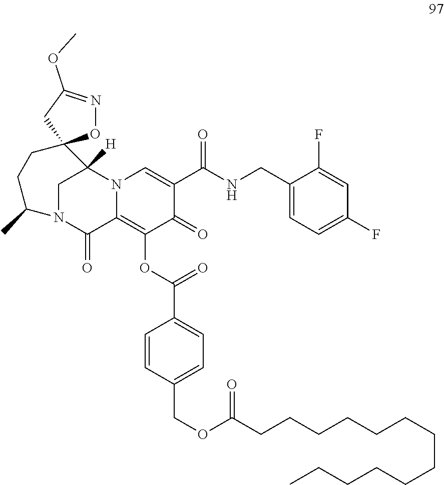 [SMILES: CCCCCCCCCCCCCC(=O)OCc1ccc(C(=O)Oc2c3n(cc(C(=O)NCc4ccc(F)cc4F)c2=O)[C@@H]2CN(C3=O)[C@@H](C)CC[C@]23CC(OC)=NO3)cc1]

The title compound was made in a manner similar to Example 91 except 4-(hydroxymethyl)benzoic acid is used instead of 2-(hydroxymethyl)benzoic acid in Step 1. MS (m/z) 847.4 [M+H]+. 1H NMR (400 MHz, Chloroform-d) δ 10.19 (t, J=5.7 Hz, 1H), 8.55 (s, 1H), 8.20 (d, J=7.4 Hz, 2H), 7.47 (d, J=8.1 Hz, 2H), 7.36 (td, J=8.3, 6.3 Hz, 1H), 6.88-6.75 (m, 2H), 5.19 (s, 2H), 4.79 (dt, J=10.4, 6.9 Hz, 1H), 4.68 (d, J=15.5 Hz, 1H), 4.60 (s, 1H), 4.26 (s, 1H), 3.94 (s, 3H), 3.83 (d, J=14.6 Hz, 1H), 3.11 (d, J=17.1 Hz, 1H), 2.78-2.65 (m, 1H), 2.40 (t, J=7.6 Hz, 2H), 2.08-1.99 (m, 1H), 2.01-1.91 (m, 2H), 1.66 (q, J=8.0 Hz, 3H), 1.26 (d, J=14.1 Hz, 24H), 0.90 (t, J=6.7 Hz, 3H).

### Example 98: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 4-((tetradecanoyloxy)methyl)benzoate (98)

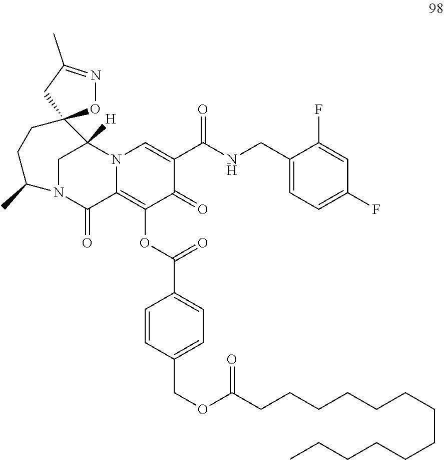 [SMILES: CCCCCCCCCCCCCC(=O)OCc1ccc(C(=O)Oc2c3n(cc(C(=O)NCc4ccc(F)cc4F)c2=O)[C@@H]2CN(C3=O)[C@@H](C)CC[C@]23CC(C)=NO3)cc1]

The title compound was made in a manner similar to Example 97, except Intermediate B was used instead of Intermediate C in step 2. MS (nm/z) 831.1 [M+H]+. 1H NMR (400) MHz, Chloroform-d) δ 10.20 (t, J=5.7 Hz, 1H), 8.46 (s, 1H), 8.21 (d, J=7.5 Hz, 2H), 7.48 (d, J=8.1 Hz, 2H), 7.36 (td, J=8.3, 6.3 Hz, 1H), 6.88-6.75 (m, 2H), 5.19 (s, 2H), 4.79 (s, 1H), 4.65 (s, 1H), 4.60 (s, 1H), 4.15-4.07 (m, 1H), 3.86-3.76 (m, 1H), 3.07 (t, J=16.3 Hz, 1H), 2.70-2.60 (in, 1H), 2.40 (t, J=7.6 Hz, 2H), 2.12 (d, J=6.9 Hz, 3H), 2.01-1.88 (m, 3H), 1.73-1.63 (m, 3H), 1.28 (s, 24H), 0.92-0.89 (m, 3H).

### Example 99: (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 4-(tetradecanoyloxy)benzoate (99)

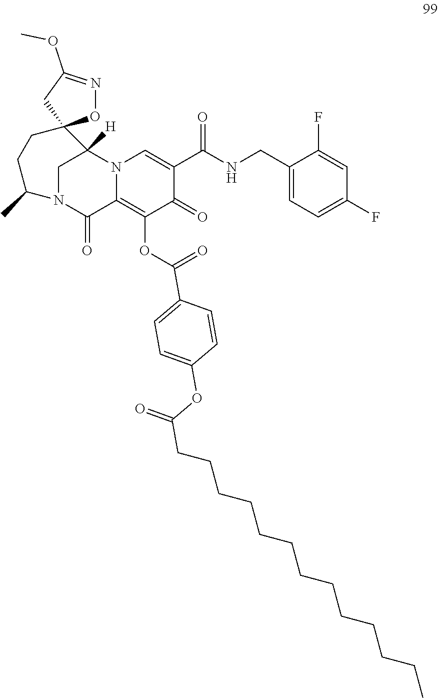 [SMILES: CCCCCCCCCCCCCC(=O)Oc1ccc(C(=O)Oc2c3n(cc(C(=O)NCc4ccc(F)cc4F)c2=O)[C@@H]2CN(C3=O)[C@@H](C)CC[C@]23CC(OC)=NO3)cc1]

The title compound was made in a manner similar to Example 95, except tert-butyl 4-hydroxybenzoate was used instead of tert-butyl 2-(4-hydroxyphenyl)acetate in Step 1. MS (m/z) 833.2 [M+H]⁺. 1H NMR (400 MHz, Chloroform-d) δ 10.28 (t, J=5.9 Hz, 1H), 8.95 (s, 1H), 7.36 (td, J=8.5, 6.3 Hz, 1H), 6.90-6.71 (m, 2H), 4.81 (dq, J=10.1, 6.7 Hz, 1H), 4.73 (dd, J=15.1, 6.2 Hz, 1H), 4.60 (s, 1H), 4.51 (dd, J=15.3, 5.4 Hz, 1H), 3.85-3.75 (m, 4H), 3.69 (dd, J=15.0, 2.6 Hz, 1H), 3.11 (d, J=16.9 Hz, 1H), 2.74-2.51 (m, 3H), 2.12-1.84 (m, 3H), 1.84-1.67 (m, 3H), 1.60 (ddd, J=15.2, 11.2, 2.2 Hz, 1H), 1.48-1.19 (m, 26H), 0.97-0.79 (m, 3H).

### Example 100: (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 4-(tetradecanoyloxy)benzoate (100)

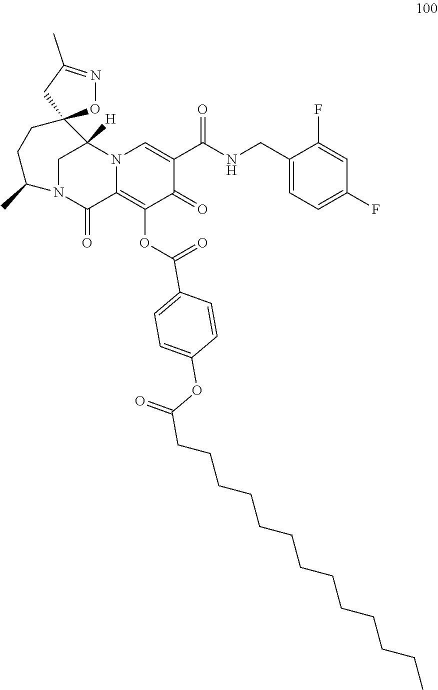 [SMILES: CCCCCCCCCCCCCC(=O)Oc1ccc(C(=O)Oc2c3n(cc(C(=O)NCc4ccc(F)cc4F)c2=O)[C@@H]2CN(C3=O)[C@@H](C)CC[C@]23CC(C)=NO3)cc1]

The title compound was made following the same method as Example 99, except using Intermediate B instead of Intermediate C in Step 3. MS (m/z) 817.10 [M+H]⁺. 1H NMR (400 MHz, Chloroform-d) δ 10.19 (t, J=6.0 Hz, 1H), 8.49 (s, 1H), 7.33 (td, J=8.7, 6.3 Hz, 1H), 6.90-6.72 (m, 2H), 4.78 (dq, J=9.9, 6.7 Hz, 1H), 4.67 (dd, J=15.1, 6.2 Hz, 1H), 4.54 (dd, J=15.1, 5.7 Hz, 1H), 4.18 (d, J=2.3 Hz, 1H), 3.71 (qd, J=15.1, 2.3 Hz, 2H), 3.04 (d, J=17.7 Hz, 1H), 2.68-2.51 (m, 3H), 2.05 (d, J=15.2 Hz, 3H), 1.98-1.81 (m, 4H), 1.76 (p, J=7.7 Hz, 2H), 1.58 (ddd, J=15.1, 9.1, 3.6 Hz, 1H), 1.44-1.20 (m, 26H), 0.93-0.80 (m, 3H).

### Example 101: (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-(tetradecanamidomethyl)benzoate (101)

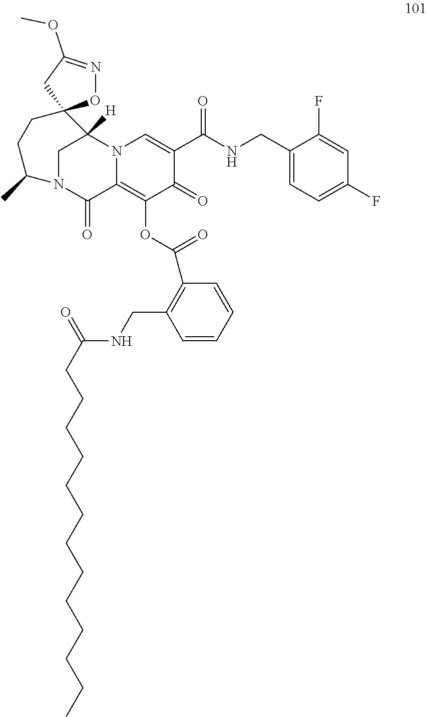 [SMILES: CCCCCCCCCCCCCC(=O)NCc1ccccc1C(=O)Oc1c2n(cc(C(=O)NCc3ccc(F)cc3F)c1=O)[C@@H]1CN(C2=O)[C@@H](C)CC[C@]12CC(OC)=NO2]

The title compound was made following the same method as Example 95, except using tert-butyl 2-(aminomethyl)benzoate instead of tert-butyl 2-(4-hydroxyphenyl)acetate in Step 1. MS (m/z) 846.2 [M+H]⁺. 1H NMR (400 MHz, Chloroform-d) δ 10.15 (t, J=6.0 Hz, 1H), 8.59 (s, 1H), 8.24-7.82 (m, 1H), 7.61 (d J=21.5 Hz, 2H), 7.45-7.34 (m, 2H), 6.94-6.71 (m, 2H), 4.95-4.52 (m, 3H), 4.29 (s, 1H), 3.95 (s, 3H), 3.86 (d, J=12.6 Hz, 1H), 3.11 (d, J=17.2 Hz, 1H), 2.76 (d, J=16.8 Hz, 1H), 2.27-1.84 (m, 5H), 1.67 (s, 7H), 1.24 (d, J=19.5 Hz, 23H), 0.90 (t, J=6.8 Hz, 3H).

### Example 102: (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-(tetradecanamidomethyl)benzoate (102)

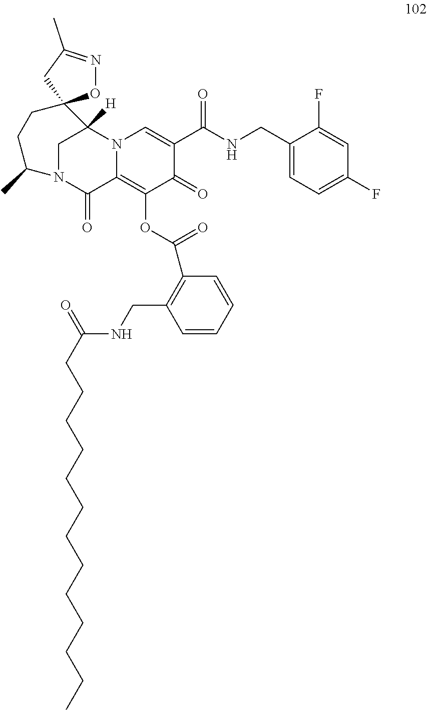 [SMILES: CCCCCCCCCCCCCC(=O)NCc1ccccc1C(=O)Oc1c2n(cc(C(=O)NCc3ccc(F)cc3F)c1=O)[C@@H]1CN(C2=O)[C@@H](C)CC[C@]12CC(C)=NO2]

The title compound was made following the same method as Example 101, except using Intermediate B instead of Intermediate C in Step 3. MS (m/z) 830.10 [M+H]⁺. 1H NMR (400 MHz, Chloroform-d) δ 10.15 (t, J=6.0 Hz, 1H), 8.52 (s, 1H), 8.23-7.82 (m, 1H), 7.68-7.50 (m, 2H), 7.50-7.32 (m, 2H), 6.91-6.70 (m, 2H), 4.89-4.47 (m, 5H), 4.18 (s, 1H), 3.78 (dd, J=53.2, 13.2 Hz, 2H), 3.09 (d, J=17.7 Hz, 1H), 2.66 (d, J=16.8 Hz, 1H), 2.24-2.07 (m, 5H), 2.07-1.88 (m, 3H), 1.83-1.50 (m, 4H), 1.34-1.13 (m, 23H), 0.89 (t, J=6.8 Hz, 3H).

### Example 103: Preparation of (3′S,5S,7′R)—N-(2,4-difluorobenzyl)-3-methoxy-3′-methyl-12′-((5-methyl-2-oxo-1,3-dioxol-4-yl)methoxy)-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (103)

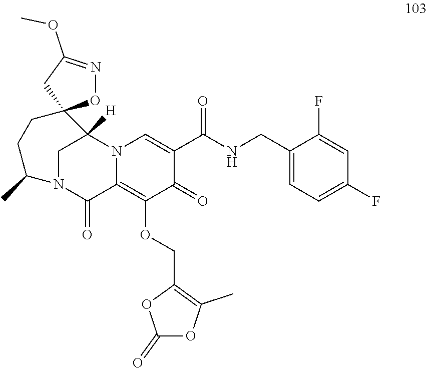 [SMILES: COC1=NO[C@@]2(CC[C@H](C)N3C[C@H]2n2cc(C(=O)NCc4ccc(F)cc4F)c(=O)c(OCc4oc(=O)oc4C)c2C3=O)C1]

The title compound was made in a manner similar to Example 22, except using Intermediate C instead of Intermediate B. MS (m/z) 615.179 [M+H]+. 1H NMR (400 MHz, Chloroform-d) δ 10.30 (t, J=59 Hz, 1H), 8.67 (s, 1H), 7.34 (td, J=8.6, 6.5 Hz, 11H), 6.89-6.72 (m, 2H), 5.19 (d, J=12.8 Hz, 1H), 5.01 (d, J=12.7 Hz, 11H), 4.89-4.77 (m, 1H), 4.67 (dd, J=15.1, 6.2 Hz, 1H), 4.54 (dd, J=15.1, 5.6 Hz, 1H), 4.43 (d, J=2.3 Hz, 1H), 3.81 (s, 3H), 3.72 (dd, J=15.3, 1.8 Hz, 1H), 3.60 (dd, J=15.2, 2.7 Hz, 1H), 3.10 (d, J=17.0 Hz, 1H), 2.61 (d, J=17.0 Hz 11H), 2.18 (s, 3H), 2.00-1.86 (m, 3H), 1.62-1.49 (m, 1H), 1.24-1.21 (m, 3H).

### Example 104: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl tetrahydrofuran-2-carboxylate (104)

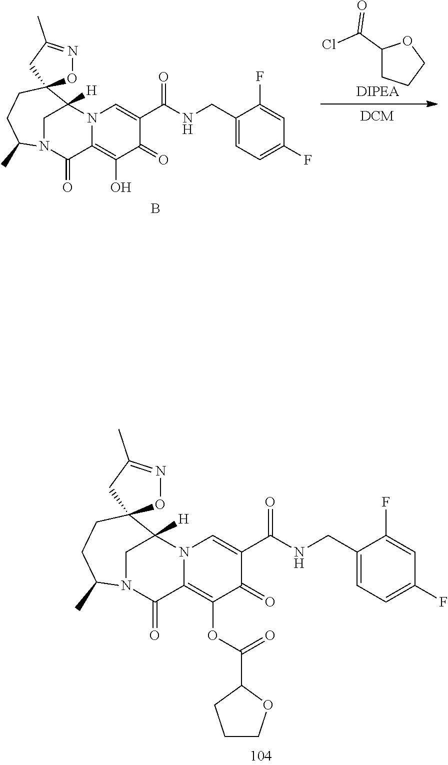

To a mixture of Intermediate B (0.300 g, 0.617 mmol) and tetrahydrofuran-2-carbonyl chloride (207 mg, 0.1.54 mmol) in DCM (5.0 mL) at room temperature was DIPEA (0.43 mL, 0.2.47 mmol). The resulting mixture was stirred at room temperature for 16 hours. The reaction was then concentrated, purified by normal phase chromatography, eluted with 0-100% EtOAc/Hexane to provide the title compound. MS (m/z) 585.0 [M+H]+, ¹H NMR (400 MHz, DMSO) δ 10.19-10.08 (m, 1H), 8.84 (s, 1H), 7.49-7.31 (m, 1H), 7.31-7.15 (m, 1H), 7.15-6.94 (m, 1H), 4.79-4.43 (m, 5H), 3.97-3.65 (m, 4H), 3.03 (d, J=17.4 Hz, 1H), 2.67 (d, J=16.8 Hz, 1H), 2.43-2.19 (m, 2H), 2.06-1.72 (m, 8H), 1.37-1.08 (m, 4H).

### Example 105: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl tetrahydrofuran-2-carboxylate (105)

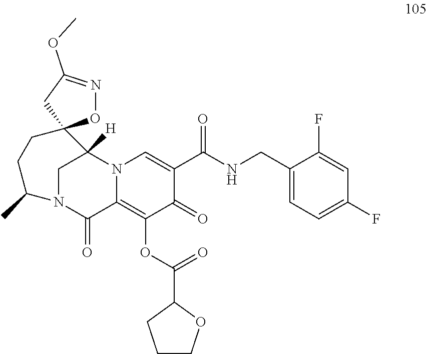 [SMILES: COC1=NO[C@@]2(CC[C@H](C)N3C[C@H]2n2cc(C(=O)NCc4ccc(F)cc4F)c(=O)c(OC(=O)C4CCCO4)c2C3=O)C1]

The title compound was prepared in a manner similar to Example 104, except using Intermediate C instead of Intermediate B. MS (m/z) 601.0 [M+H]+, ¹H NMR (400 MHz, DMSO) δ 10.22-10.00 (m, 1H), 8.88 (s, 1H), 7.50-7.35 (m, 1H), 7.35-7.15 (m, 1H), 7.15-6.98 (m, 1H), 4.77-4.53 (m, 5H), 3.89-3.65 (m, 6H), 3.08 (d, J=16.8 Hz, 1H), 2.79 (d, J=17.3 Hz, 1H), 2.36-2.24 (m, 1H), 1.97-1.78 (m, 6H), 1.28-1.13 (m, 5H).

### Example 106: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 3-methyltetrahydrofuran-2-carboxylate (106)

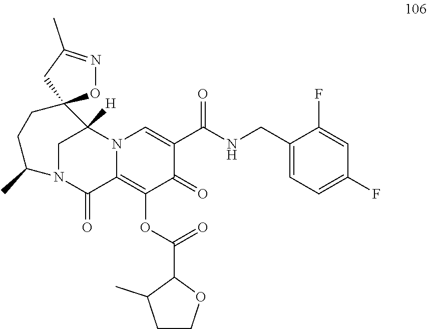 [SMILES: CC1=NO[C@@]2(CC[C@H](C)N3C[C@H]2n2cc(C(=O)NCc4ccc(F)cc4F)c(=O)c(OC(=O)C4OCCC4C)c2C3=O)C1]

The title compound was prepared in a manner similar to Example 104, except using 3-methyltetrahydrofuran-2-carbonyl chloride instead of tetrahydrofuran-2-carbonyl chloride. MS (m/z) 599.0 [M+H]+, ¹H NMR (400 MHz, DMSO) δ 10.36-10.23 (m, 1H), 8.90-8.75 (m, 1H), 7.48-7.35 (m, 1H), 7.35-7.19 (m, 1H), 7.19-6.99 (m, 1H), 4.73-4.36 (m, 6H), 3.84-3.53 (m, 4H), 3.02 (d, J=17.4 Hz, 1H), 2.81-2.61 (m, 2H), 2.03-1.87 (m, 3H), 1.89-1.63 (m, 3H), 1.42-1.15 (m, 8H).

### Example 107: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 3-methyltetrahydrofuran-2-carboxylate (107)

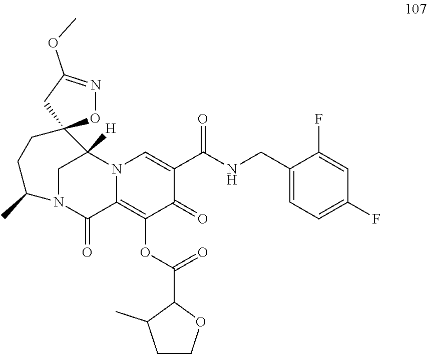 [SMILES: COC1=NO[C@@]2(CC[C@H](C)N3C[C@H]2n2cc(C(=O)NCc4ccc(F)cc4F)c(=O)c(OC(=O)C4OCCC4C)c2C3=O)C1]

The title compound was prepared in a manner similar to Example 106, except using Intermediate C instead of Intermediate B. MS (m/z) 614.9 [M+H]+, ¹H NMR (400 MHz, DMSO) δ 10.19-10.03 (m, 1H), 8.92 (s, 1H), 7.51-7.39 (m, 1H), 7.36-7.16 (m, 1H), 7.16-6.98 (m, 1H), 4.92-4.69 (m, 1H), 4.69-4.43 (m, 4H), 3.87-3.57 (m, 6H), 3.18-3.03 (m, 1H), 2.81 (d, J=17.0 Hz, 1H), 1.96-1.72 (m, 4H), 1.36-1.11 (m, 10H).

### Example 108: HIV MT-4 Antiviral and Cytotoxicity Assay

### Antiviral Assay in MT-4 Cells

Compounds were tested in a high-throughput 384-well assay format for their ability to inhibit the replication of HIV-1 (IIIB) in MT-4 cells. Compounds were serially diluted (1.3) in DMSO on 384-well polypropylene plates and further diluted 200-fold into complete RPMI media (10% FBS, 1% P/S) using the Biotek Micro Flow and Labcyte ECHO acoustic dispenser. Each plate contained up to 8 test compounds, with negative (No Drug Control) and 5 μAZT positive controls. MT-4 cells were pre-infected with 10 μL of either RPMI (mock-infected) or a fresh 1:250 dilution of HIV-1 IIIB concentrated virus stock. Infected and uninfected MT-4 cells were further diluted in complete RPMI media and added to each plate using a Micro Flow dispenser. After 5 days incubation in a humidified and temperature controlled incubator (37° C.), Cell Titer Glo (Promega) was added to the assay plates and chemiluminescence read using an Envision plate-reader. EC₅₀ values were defined as the compound concentration that causes a 50% decrease in luminescence signal, and were calculated using a sigmoidal dose-response model to generate curve fits. The EC₅₀ data for exemplary compounds is shown in Table 1.

### Cytotoxicity Assay in MT-4 Cells

Assays were performed as above except uninfected MT-4 cells were added to each well containing test compound. In addition, 10 μM puromycin was added to the last column of each assay plate to assess a base level of cytotoxicity. The CC₅₀ data for exemplary compounds is shown in Table 1.

TABLE 1

| Compound | MT4 | MT4 |
|---|---|---|
| No. | EC₅₀ (nM) | CC₅₀ (nM) |
| 1 | 3.479 | 15893 |
| 2 | 1.492 | 10238 |
| 3 | 2.15 | 12629 |
| 4 | 14.837 | 50000 |
| 5 | 1.241 | 10256 |
| 6 | 0.917 | 10081 |
| 7 | 1.476 | 10779 |
| 8 | 0.982 | 10396 |
| 9 | 1.4 | 10490 |
| 10 | 3.188 | 10207 |
| 11 | 1.902 | 14473 |
| 12 | 4.326 | 14596 |
| 13 | 0.762 | 10800 |
| 14 | 1.25 | 16758 |
| 15 | 1.614 | 14665 |
| 16 | 2.927 | 15951 |
| 17 | 0.93 | 12971 |
| 18 | 12.277 | 50000 |
| 19 | 0.71 | 10947 |
| 20 | 1.765 | 12482 |
| 21 | 2.537 | 12588 |
| 22 | 1.177 | 12381 |
| 23 | 2.108 | 14577 |
| 24 | 1.979 | 12258 |
| 25 | 1.432 | 11562 |
| 26 | 1.134 | 12225 |
| 27 | 2.751 | 16852 |
| 29 | 2.011 | 27766 |
| 33 | 2.337 | 12851 |
| 34 | 1.4 | 12772 |
| 35 | 2.035 | 11597 |
| 36 | 3.087 | 22009 |
| 37 | 13.273 | 33497 |
| 38 | 2.024 | 17092 |
| 39 | 88.658 | 50000 |
| 40 | 100 | 50000 |
| 41 | 0.727 | 11962 |
| 42 | 1.902 | 15727 |
| 43 | 2.638 | 30730 |
| 44 | 1.8 | 13548 |
| 45 | 1.73 | 11445 |
| 46 | 2.514 | 15078 |
| 47 | 2.219 | 11169 |
| 48 | 3.818 | 15738 |
| 49 | 4.173 | 23288 |
| 50 | 1.69 | 21636 |
| 51 | 2.005 | 21219 |
| 52 | 1.465 | 13011 |
| 53 | 4.963 | 35654 |
| 54 | 9.287 | 50000 |
| 55 | 2.5 | 12931 |
| 56 | 2.399 | 12791 |
| 57 | 2.546 | 14950 |
| 58 | 5.778 | 28994 |
| 59 | 1.2 | 10556 |
| 60 | 1.588 | 13751 |
| 61 | 1.394 | 15162 |
| 62 | 1.725 | 14612 |
| 63 | 18.711 | 50000 |
| 64 | 21.398 | 50000 |
| 65 | 23.288 | 50000 |
| 66 | 63.844 | 50000 |
| 67 | 21.003 | 39004 |
| 68 | 0.721 | 12980 |
| 69 | 1.779 | 14124 |
| 70 | 1.626 | 10154 |
| 71 | 1.016 | 9303.7 |
| 72 | 1.338 | 11238 |
| 73 | 0.634 | 8483.6 |
| 74 | 6.611 | 29425 |
| 75 | 0.691 | 9190.1 |
| 76 | 4.405 | 12915 |
| 77 | 2.048 | 19412 |
| 78 | 2.202 | 8045.6 |
| 79 | 2.232 | 12484 |
| 80 | 0.785 | 10127 |
| 81 | 2.094 | 14439 |
| 82 | 2.168 | 8518 |
| 83 | 1.356 | 13151 |
| 84 | 38.045 | 50000 |
| 85 | 1.424 | 8343.9 |
| 86 | 0.992 | 10531 |
| 87 | 2.385 | 10634 |
| 88 | 2.192 | 11114 |
| 89 | | |
| 90 | | |
| 91 | 5.068 | 50000 |
| 92 | 4.417 | 50000 |
| 93 | 1.365 | 50000 |
| 94 | 3.204 | 50000 |
| 95 | 3.493 | 50000 |
| 96 | 2.264 | 50000 |
| 97 | | |
| 98 | | |
| 99 | 3.032 | 50000 |
| 100 | 2.935 | 12822 |
| 101 | | 50000 |
| 102 | | 31774 |
| 103 | 1.91 | 18914 |
| 104 | | |
| 105 | | |
| 106 | | |
| 107 | | |

### Example 109: Thermodynamic Solubility Assay

Approximately 7 mg of each test compound as a dry powder was placed in a vial. Aliquots were weighed out for each assay media at 2 hour and 24 hour time points, to be analyzed. The appropriate buffer (FaSSiF or PBS) was added to each vial such that the final dose concentration of 5 mg/nL was achieved. Samples were then vortexed for 5-10 seconds. Following a 2-hour or a 24-hour incubation on a rotary shaker (200 RPM) at ambient temperature (22.3-23.8° C.), the samples were vacuum filtered through a Millipore solubility filter plate with 0.45 μM polycarbonate filter membrane and the filtrates were collected in a 96 well polypropylene plate. The plate was sealed with a pierceable heat seal and analyzed by HPLC-UV.

An Agilent 1290 UHPLC equipped with a micro-well plate autosampler, quaternary HPLC pump, and diode array detector was used for analysis. Each filtrate (1.5 μL) was injected onto the column (AQUASIL C18, 5 μM, 50×2.1 mm) and eluted using a gradient of 10-100% MeCN/water containing 0.1% formic acid. A system QC (0.01 mg/mL Caffeine in DMSO) was injected every 12-15 injections. Data was collected at 214, 254 and 280 nm. Results are reported using data obtained at 280 nm. The resulting peak areas were plotted against the known concentrations from the calibration and the filtrates are quantified with respect to the linear regression using Agilent OpenLab Intelligent Reporting Software. The results are reported electronically in mg/nL for exemplary compounds in Table 2. Comparative results for Intermediate B and Intermediate C are also included in Table 2.

TABLE 2

| | Thermodynamic |
|---|---|
| | FaSSIF |
| Example | Solubility @ |
| No. | 24 h (mg/mL) |
| 1 | 4.905 |
| 2 | 4.952 |
| 3 | 5.41 |
| 4 | 4.8 |
| 5 | 4.015 |
| 6 | 4.81 |
| 7 | 4.655 |
| 8 | 4.8 |
| 9 | 3.235 |
| 10 | 3.325 |
| 11 | 0.002 |
| 12 | 2.705 |
| 13 | 1.98 |
| 14 | 5.09 |
| 15 | 1.475 |
| 16 | |
| 17 | 2.545 |
| 18 | 4.33 |
| 19 | 0.39 |
| 20 | 4.41 |
| 21 | 3.75 |
| 22 | 0.09 |
| 23 | 4.75 |
| 24 | 4.895 |
| 25 | 4.76 |
| 26 | 4.295 |
| 27 | 2.26 |
| 28 | |
| 29 | 2.465 |
| 30 | |
| 31 | 3.75 |
| 32 | 1.33 |
| 33 | 4.17 |
| 34 | 4.925 |
| 35 | 4.46 |
| 36 | |
| 37 | 4.495 |
| 38 | 5.555 |
| 39 | 4.83 |
| 40 | 5.01 |
| 41 | 5.08 |
| 42 | |
| 43 | 5.28 |
| 44 | 4.44 |
| 45 | 5.325 |
| 46 | 4.85 |
| 47 | 4.855 |
| 48 | 4.915 |
| 49 | 4.89 |
| 50 | |
| 51 | 4.395 |
| 52 | 3.835 |
| 53 | 4.68 |
| 54 | 5.28 |
| 55 | 5.525 |
| 56 | 3.565 |
| 57 | 5.455 |
| 58 | 4.645 |
| 59 | 5.275 |
| 60 | 5.04 |
| 61 | 4.145 |
| 62 | 5.03 |
| 63 | 5 |
| 64 | 4.84 |
| 65 | |
| 66 | 4.825 |
| 67 | 5.47 |
| 68 | 4.63 |
| 69 | 5.135 |
| 70 | 2.465 |
| 71 | 3.47 |
| 72 | 2.56 |
| 73 | 5.07 |
| 74 | 4.865 |
| 75 | 4.74 |
| 76 | 4.06 |
| 77 | |
| 78 | 5.135 |
| 79 | 4.515 |
| 80 | 4.15 |
| 81 | |
| 82 | 4.62 |
| 83 | 4.12 |
| 84 | 5.575 |
| 85 | 4.725 |
| 86 | 4.9 |
| 87 | |
| 88 | |
| 89 | |
| 90 | |
| 91 | 0.0015 |
| 92 | 0.003 |
| 93 | 0.006 |
| 94 | 0.019 |
| 95 | 0.0145 |
| 96 | 0.0135 |
| 97 | |
| 98 | |
| 99 | 0.013 |
| 100 | 0.0645 |
| 101 | |
| 102 | |
| 103 | |
| 104 | |
| 105 | |
| 106 | |
| Intermediate B | 0.002 |
| Intermediate C | 0.002 |

FaSSIF = Fasted-state simulated intestinal fluid.

### Example 110: Pharmacokinetic Studies

Doses are expressed as “mg-eq”, referring to the mass (in mg) of Intermediate B or Intermediate C equivalents (eq) as a fixed dose or relative to body weight (in kg).

### Dog PK Dosing

Male beagle dogs were fasted overnight. Food was returned approximately 4 hours postdose. In some instances, each animal received a single 6 μg/kg intramuscular injection of pentagastrin approximately 30 minutes prior to test article administration to stimulate gastric secretion. The intramuscular dose was administered in a thigh muscle using a needle and syringe.

Solution or suspension oral doses were administered at a dose of 4 mg-eq/kg via gavage and the dosing tube was flushed with ˜10 mL water prior to removal. Solid or capsule oral doses were administered at a dose of 40 mg-eq fixed, by hand by deep throat deposition. Following each dose, the animals were offered approximately 5 mL of water to assist in swallowing. This was done by depositing ˜1-3 mL at a time into the back of the throat and holding the mouth closed until swallowing was observed. This was repeated until all 5 mL of water has been given. Once swallowed, 25 mL water was administered by gavage for a total of 30 mL water administered with each dose. All animals were observed at dosing and each scheduled collection.

Serial blood samples (through 168 h) were collected into pre-chilled K2EDTA with the appropriate volume of 40 mM dichlorvos added to result in a final dichlorvos concentration of 2 mM and stored on wet ice until processed. Whole blood was processed to plasma by centrifugation (3500 rpm for 10 minutes at 5° C.) within 30 minutes of collection. Plasma samples were transferred into Micronic 96 well tubes and stored at −70° C. as soon as possible and remained at −70° C. until shipped for bioanalysis.

### Bioanalysis of Plasma Samples

To a 20 μL aliquot of each plasma sample with exception of the matrix blanks, 120 μL of 100 ng/mL Carbamazepine and Chrysin in acetonitrile (ACN) was added. The matrix blank samples received 120 μL of acetonitrile only. The precipitated proteins were removed by centrifugation and 100 μL of supernatant was transferred into a clean 96-well plate. A 100 μL aliquot of water was added to each sample. An aliquot of 2-2.5 μL was injected into an Applied Biosystems API-6500 LC/MS/MS system, eluting with a gradient of water and acetonitrile (containing 0.1% formic acid).

AUC_{inf} was calculated as area under the plasma concentration vs. time curve from 0 h to infinity.

Bioavailability (% F) was calculated by comparing plasma concentration via PO dose (oral) vs. plasma concentration via IV dose (intravenous) using the following equation:

$$\% F = \frac{PO AUCinf}{IV AUCinf} \times \frac{IV Dose}{PO Dose} \times 100$$

TABLE 3
Oral Bioavailability of Intermediate B in Dog Following
a 4 mg-eq/kg Solution Dose of Test Compound.

| | | | Dog % F of |
|---|---|---|---|
| | | | Intermediate B |
| | Compound | Compound | (4 mg-eq/kg |
| Entry | Dosed | Measured | solution) |
| 1 | 1 | Intermediate B | 2.3 |
| 2 | 2 | Intermediate B | 10.3 |
| 3 | 9 | Intermediate B | 6.4 |

TABLE 4
Oral Bioavailability of Intermediate B in Dog
Following a 40 mg-eq Powder-in-Capsule
Fixed Dose of Test Compound.

| | | | Dog % F of |
|---|---|---|---|
| | Compound | Compound | Intermediate B |
| Entry | Dosed | Measured | (40 mg-eq PIC) |
| 1 | 2 | Intermediate B | 8.4 |
| 2 | 6 | Intermediate B | 32.2 |
| 3 | 7 | Intermediate B | 42.9 |
| 4 | 14 | Intermediate B | 6.7 |
| 5 | 23 | Intermediate B | 4 |
| 6 | 26 | Intermediate B | 6.4 |
| 7 | 27 | Intermediate B | 40.8 |

TABLE 5
Oral Bioavailability of Intermediate B in Dog Following
a 175 mg-eq Tablet Fixed Dose of Test Compound.

| | | | Dog % F of |
|---|---|---|---|
| | Compound | Compound | Intermediate B |
| Entry | Dosed | Measured | (175 mg-eq Tablet) |
| 1 | 4 | Intermediate B | 2.1 |
| 2 | 8 | Intermediate B | 20.9 |
| 3 | 24 | Intermediate B | 1.8 |
| 4 | 43 | Intermediate B | 6.7 |
| 5 | 47 | Intermediate B | 41.4 |
| 6 | 61 | Intermediate B | 13.2 |
| 7 | 69 | Intermediate B | 8.9 |
| 8 | 73 | Intermediate B | 1.1 |
| 9 | 85 | Intermediate B | 20.1 |

TABLE 6
Oral Bioavailability of Intermediate B or Intermediate
C in Dog Following a 300 mg-eq Powder-in-Capsule or
Tablet Fixed Dose of Test Compound.

| | | | Dog % F of |
|---|---|---|---|
| | | | Intermediate B or |
| | | | Intermediate C |
| | Compound | Compound | (300 mg-eq |
| Entry | Dosed | Measured | PIC or Tablet) |
| 1 | 6 | Intermediate B | 15.6 |
| 2 | 7 | Intermediate B | 14.5 |
| 3 | 20 | Intermediate B | 6.4 |
| 4 | 21 | Intermediate C | 8.5 |
| 5 | 27 | Intermediate B | 9.1 |

TABLE 7
Oral Bioavailability of Intermediate B or Intermediate
C in Dog Following a 175 mg Tablet Fixed Dose.

| | | Dog % F |
|---|---|---|
| Entry | Compound Dosed | (175 mg Tablet) |
| 1 | Intermediate B | 15.6 |
| 2 | Intermediate C | 14.5 |

All of the U.S. patents, U.S. patent application publications, U.S. patent applications, foreign patents, foreign patent applications and non-patent publications referred to in this specification are incorporated herein by reference, in their entirety to the extent non inconsistent with the present description.

From the forgoing it will be appreciated that, although specific embodiments have been described herein for purposes of illustration, various modifications may be made without deviating from the spirit and scope of the disclosure. Accordingly, the disclosure is not limited except as by the appended claims.

1. A compound of Formula I: 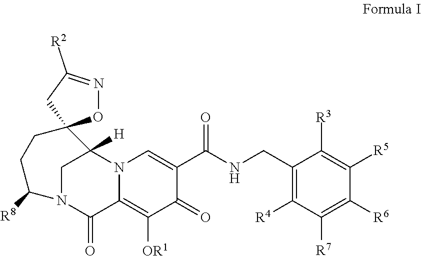
or a pharmaceutically acceptable salt thereof, wherein:
R¹ is —(CR^{1A}R^{1B}O)ₐ(Y)_{b}(CR^{1C}R^{1D})_{d}X
wherein a is 0 or 1;
b is 0 or 1;
d is 0, 1,2 or 3;
R^{1A} is H or C₁₋₃alkyl;
R^{1B} is H or C₁₋₃alkyl;
each R^{1C} is independently H or C₁₋₃alkyl;
each R^{1D} is independently H or C₁₋₃alkyl;
Y is —C(O)—, —C(O)O—, —C(O)NH—, —C(O)NR^{1L}—, or P(O)(OH)O—;
R^{1L} is a C₁₋₄alkyl optionally substituted with one or two substituents independently selected from a group consisting of —COOH, —OH, —NH₂, and —CONH₂;
X is selected from the group consisting of:
(a) phenyl or pyridyl; wherein the phenyl or pyridyl is optionally substituted with one, two or three substituents independently selected from the group consisting of —OP(O)(OR^{1E})₂, R^{1F}, —COOH, —OCO—C₁₋₂₀alkyl, —CONR^{1X}R^{1Y}, and —NHCO—C₁₋₂₀alkyl;
wherein each R^{1E} is independently H or phenyl;
each R^{1F} is independently a C₁₋₃alkyl optionally substituted with one or two substituents independently selected from the group consisting of —COOH, —P(O)(OH)₂, —OP(O)(OR^{1E})₂, —NR^{1G}R^{1H}, —CONR^{1G}R^{1H}, —OCOR^{1I}, and —NHCOR^{1I};
each R^{1G} is independently H, —COO(CR^{1N}R^{1O})ₑOP(O)(OH)₂, or C₁₋₄alkyl; wherein the C₁₋₄ alkyl is optionally substituted with one, two, or three substituents independently selected from a group consisting of halo, —COOR^{1M}, —OR^{1W}, —P(O)(OH)₂, —NH₂, —NR^{1J}R^{1K}, and —CONR^{1J}R^{1K};
each R^{1H} is independently H, —COO(CR^{1N}R^{1O})ₑOP(O)(OH)₂, or C₁₋₄alkyl; wherein the C₁₋₄ alkyl is optionally substituted with one, two, or three substituents independently selected from a group consisting of halo, —COOR^{1M}, —OR^{1W}, —P(O)(OH)₂, —NH₂, —NR^{1J}R^{1K}, and —CONR^{1J}R^{1K}; or
optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
each R^{1I} is independently C₁₋₂₀alkyl optionally substituted with one or two substituents selected independently from —P(O)(OH)₂, —COOH and —NR^{1J}R^{1K};
each R^{1J} is independently H or C₁₋₃alkyl, wherein the C₁₋₃alkyl is optionally substituted with one or two —COOH;
each R^{1K} is independently H or C₁₋₃alkyl, wherein the C₁₋₃alkyl is optionally substituted with one or two —COOH;
each R^{1M} is independently H or C₁₋₄alkyl, wherein the C₁₋₄alkyl is optionally substituted with phenyl;
each R^{1N} is independently H or C₁₋₃alkyl;
each R^{1O} is independently H or C₁₋₃alkyl;
each R^{1W} is independently H or C₁₋₃alkyl;
e is 1, 2, or 3;
each R^{1X} is H or C₁₋₄alkyl; wherein the C₁₋₄alkyl is optionally substituted with one or two —COOH; and
each R^{1Y} is H or C₁₋₄alkyl; wherein the C₁₋₄alkyl is optionally substituted with one or two —COOH;
(b) 4 to 7 membered heterocyclyl comprising 1, 2 or 3 heteroatoms selected from N, O, and S; wherein the heterocyclyl is optionally substituted with one, two or three substituents independently selected from the group consisting of C₁₋₄alkyl, —COOC₁₋₄alkyl, oxo, and —COOCH₂OP(O)(OH)₂, wherein the C₁₋₄alkyl is optionally substituted with —OP(O)(OH)₂ or —NR^{1Z}R^{1AA};
wherein R^{1Z} is H, —COO(CR^{1AB}R^{1AC})ₕOP(O)(OH)₂ or C₁₋₄alkyl, wherein the C₁₋₄ alkyl is optionally substituted with one or two —COOH;
R^{1AA} is H, —COO(CR^{1AD}R^{1AE})ᵢOP(O)(OH)₂ or C₁₋₄alkyl, wherein the C₁₋₄ alkyl is optionally substituted with one or two —COOH;
each R^{1AB} is independently H or C₁₋₃alkyl;
each R_{1AC} is independently H or C₁₋₃alkyl;
each R^{1AD} is independently H or C₁₋₃alkyl;
each R^{1AE} is independently H or C₁₋₃alkyl;
h is 1, 2, or 3; and
i is 1, 2, or 3; and
(c) C₃₋₇ cycloalkyl optionally substituted with one, two, or three substituents independently selected from the group consisting of C₁₋₄alkyl, —COOC₁₋₄alkyl, oxo, —COOR^{1P}, and —OP(O)(OH)₂, wherein the C₁₋₄alkyl is optionally substituted with —OP(O)(OH)₂ or —NR^{1Q}R^{1R};
wherein R^{1P} is H or C₁₋₃ alkyl;
R^{1Q} is H, —COO(CR^{1S}R^{1T})_{f}OP(O)(OH)₂, or C₁₋₄alkyl, wherein the C₁₋₄alkyl is optionally substituted with —COOH;
R^{1R} is H, —COO(CR^{1U}R^{1V})_{g}OP(O)OH)₂, or C₁₋₄alkyl, wherein the C₁₋₄alkyl is optionally substituted with —COOH;
each R^{1S}, R^{1T}, R^{1U}, and R^{1V} is independently H or C₁₋₃alkyl;
f is 1, 2, or 3; and
g is 1, 2, or 3;
R² is C₁₋₃ alkyl or C₁₋₃ alkoxy;
each R³, R⁴, R⁵, R⁶ and R⁷ is independently H or halo; and
R⁸ is H or C₁₋₃alkyl.

2. A compound of Formula I: 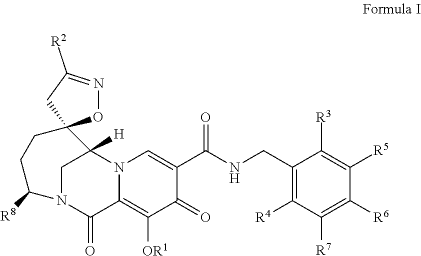
or a pharmaceutically acceptable salt thereof, wherein:
R¹ is —(CR^{1A}R^{1B}O)ₐ(Y)_{b}(CR^{1C}R^{1D})_{d}X
wherein a is 0 or 1;
b is 0 or 1;
d is 0, 1,2 or 3;
R^{1A} is H or C₁₋₃alkyl;
R^{1B} is H or C₁₋₃alkyl;
each R^{1C} is independently H or C₁₋₃alkyl;
each R^{1D} is independently H or C₁₋₃alkyl;
Y is —C(O)—, —C(O)O—, —C(O)NH—, —C(O)NR^{1L}, or P(O)(OH)O—;
R^{1L} is a C₁₋₄alkyl optionally substituted with one or two substituents independently selected from a group consisting of —COOH, —OH, —NH₂, and —CONH₂;
X is selected from the group consisting of:
(a) phenyl or pyridyl; wherein the phenyl or pyridyl is optionally substituted with one, two or three substituents independently selected from the group consisting of —OP(O)(OR^{1E})₂, R^{1F}, —COOH, —OCO—C₁₋₂₀alkyl, —NHCO—C₁₋₂₀alkyl,
wherein each R^{1E} is independently H or phenyl;
each R^{1F} is independently a C₁₋₃alkyl optionally substituted with one or two substituents independently selected from the group consisting of —COOH, —P(O)(OH)₂, —OP(O)(OR^{E})₂, —NR^{1G}R^{1H}, —CONR^{1G}R_{1H}, —OCOR^{1I}, and —NHCOR^{1I};
each R^{1G} is independently H, —COO(CR^{1N}R^{1O})ₑOP(O)(OH)₂, or C₁₋₄alkyl; wherein the C₁₋₄ alkyl is optionally substituted with one or two substituents independently selected from a group consisting of —COOR^{1M}, —OH, —NH₂, —NR^{1J}R^{1K}, and —CONR^{1J}R^{1K};
each R^{1H} is independently H, —COO(CR^{1N}R^{1O})ₑOP(O)(OH)₂, or C₁₋₄alkyl; wherein the C₁₋₄ alkyl is optionally substituted with one or two substituents independently selected from a group consisting of —COOR^{1M}, —OH, —NH₂, —NR^{1J}R^{1K}, and —CONR^{1J}R^{1K}; or
optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
each R^{1I} is independently C₁₋₂₀alkyl optionally substituted with one or two substituents selected independently from —COOH and —NR^{1J}R^{1K};
each R^{1J} is independently H or C₁₋₃alkyl, wherein the C₁₋₃alkyl is optionally substituted with one or two —COOH;
each R^{1K} is independently H or C₁₋₃alkyl, wherein the C₁₋₃alkyl is optionally substituted with one or two —COOH;
each R^{1M} is independently H or C₁₋₄alkyl, wherein the C₁₋₄alkyl is optionally substituted with phenyl;
each R^{1N} is independently H or C₁₋₃alkyl;
each R^{1O} is independently H or C₁₋₃alkyl;
e is 1, 2, or 3;
(b) 4 to 7 membered heterocyclyl comprising 1, 2 or 3 heteroatoms selected from N, O, and S; wherein the heterocyclyl is optionally substituted with one, two or three substituents independently selected from the group consisting of C₁₋₄alkyl, —COOC₁₋₄alkyl, oxo, and —COOCH₂OP(O)(OH)₂, wherein the C₁₋₄alkyl is optionally substituted with —OP(O)(OH)₂; and
(c) C₃₋₆ cycloalkyl optionally substituted with one, two or three substituents independently selected from the group consisting of C₁₋₄alkyl, —COOC₁₋₄alkyl, oxo, and —OP(O)(OH)₂, wherein the C₁₋₄alkyl is optionally substituted with —OP(O)(OH)₂;
R² is C₁₋₃ alkyl or C₁₋₃ alkoxy;
each R³, R⁴, R⁵, R⁶ and R⁷ is independently H or halo; and
R⁸ is H or C₁₋₃alkyl.

3. The compound of claim 1, or the pharmaceutically acceptable salt thereof, wherein R⁴, R⁵ and R⁷ are each H.

4. The compound of claim 1, or the pharmaceutically acceptable salt thereof, wherein R³ and R⁶ are each independently a halo.

5. (canceled)

6. The compound of claim 1, or the pharmaceutically acceptable salt thereof, wherein R^{1L} is —CH₃.

7. The compound of claim 1, or the pharmaceutically acceptable salt thereof, wherein Y is —C(O)—, —C(O)O—, —C(O)NH—, —C(O)NCH₃, or —P(O)(OH)O—.

8.-10. (canceled)

11. The compound of claim 1, or the pharmaceutically acceptable salt thereof, wherein Y is —C(O)NR^{1L}.

12.-13. (canceled)

14. The compound of claim 1, or the pharmaceutically acceptable salt thereof, wherein R⁸ is C₁₋₃alkyl.

15. (canceled)

16. The compound of claim 1, or the pharmaceutically acceptable salt thereof, wherein R² is methyl or methoxy.

17.-18.

19. The compound of claim 1, or the pharmaceutically acceptable salt thereof, wherein X is phenyl or pyridyl; wherein the phenyl or pyridyl is optionally substituted with one, two or three substituents independently selected from the group consisting of —OP(O)(OR^{1E})₂, R^{1F}, —COOH, —OCO—C₁₋₂₀alkyl, —CONR^{1X}R^{1Y}, and —NHCO—C₁₋₂₀alkyl;
wherein each R^{1E} is independently H or phenyl;
each R^{1F} is independently a C₁₋₃alkyl optionally substituted with one or two substituents independently selected from the group consisting of —COOH, —P(O)(OH)₂, —OP(O)(OR^{1E})₂, —NR^{1G}R^{1H}, —CONR^{1G}R^{1H}, —OCOR^{1I}, and —NHCOR^{1I};
each R^{1G} is independently H, —COO(CR^{1N}R^{1O})ₑOP(O)(OH)₂, or C₁₋₄alkyl; wherein the C₁₋₄ alkyl is optionally substituted with one, two, or three substituents independently selected from a group consisting of halo, —COOR^{1M}, —OR^{1W}, —P(O)(OH)₂, —NH₂, —NR^{1J}R^{1K}, and —CONR^{1J}R^{1K};
each R^{1H} is independently H, —COO(CR^{1N}R^{1O})ₑOP(O)(OH)₂, or C₁₋₄alkyl; wherein the C₁₋₄ alkyl is optionally substituted with one, two, or three substituents independently selected from a group consisting of halo, —COOR^{1M}, —OR^{1W}, —P(O)(OH)₂, —NH₂, —NR^{1J}R^{1K}, and —CONR^{1J}R^{1K}; or
optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
each R^{1I} is independently C₁₋₂₀alkyl optionally substituted with one or two substituents selected independently from —P(O)(OH)₂, —COOH and —NR^{1J}R^{1K};
each R^{1J} is independently H or C₁₋₃alkyl, wherein the C₁₋₃alkyl is optionally substituted with one or two —COOH;
each R^{1K} is independently H or C₁₋₃alkyl, wherein the C₁₋₃alkyl is optionally substituted with one or two —COOH;
each R^{1M} is independently H or C₁₋₄alkyl, wherein the C₁₋₄alkyl is optionally substituted with phenyl;
each R^{1N} is independently H or C₁₋₃alkyl;
each R^{1O} is independently H or C₁₋₃alkyl;
each R^{1W} is independently H or C₁₋₃alkyl;
e is 1, 2, or 3;
each R^{1X} is H or C₁₋₄alkyl; wherein the C₁₋₄alkyl is optionally substituted with one or two —COOH; and
each R^{1Y} is H or C₁₋₄alkyl; wherein the C₁₋₄alkyl is optionally substituted with one or two —COOH.

20. (canceled)

21. The compound of claim 1, or the pharmaceutically acceptable salt thereof, wherein X is phenyl; wherein the phenyl is optionally substituted with one, two or three substituents independently selected from the group consisting of —OP(O)(OR^{1E})₂, R^{1F}, —COOH, —OCO—C₁₋₂₀alkyl, —CONR^{1X}R^{1Y}, and —NHCO—C₁₋₂₀alkyl,
wherein each R^{1E} is independently H or phenyl;
each R^{1F} is independently a C₁₋₃alkyl optionally substituted with one or two substituents independently selected from the group consisting of —COOH, —P(O)(OH)₂, —OP(O)(OR^{1E})₂, —NR^{1G}R^{1H}, —CONR^{1G}R^{1H}, —OCOR^{1I}, and —NHCOR^{1I};
each R^{1G} is independently H, —COOCH₂OP(O)(OH)₂, or C₁₋₄alkyl; wherein the C₁₋₄ alkyl is optionally substituted with one, two, or three substituents independently selected from a group consisting of halo, —COOH, —OR^{1W}, —P(O)(OH)₂, —NH₂, —CONH₂ and —NR^{1J}R^{1K};
each R^{1H} is independently H, —COOCH₂OP(O)(OH)₂, or C₁₋₄alkyl; wherein the C₁₋₄ alkyl is optionally substituted with one, two, or three substituents independently selected from a group consisting of halo, —COOH, —OR^{1W}, —P(O)(OH)₂, —NH₂, —CONH₂ and —NR^{1J}R^{1K}; or
optionally R^{1G} and R^{1H} are joined to form a 4 to 6 membered heterocycle comprising 1, 2, or 3 heteroatoms selected from N, O, and S; wherein the 4 to 6 membered heterocycle is optionally substituted with one or two substituents independently selected from the group consisting of —OH and —COOH;
each R^{1I} is independently C₁₋₂₀alkyl optionally substituted with one or two substituents selected independently from —P(O)(OH)₂, —COOH and —NR^{1J}R^{1K};
each R^{1J} is independently H or C₁₋₃alkyl wherein the C₁₋₃alkyl is optionally substituted with one or two —COOH;
each R^{1K} is independently H or C₁₋₃alkyl wherein the C₁₋₃alkyl is optionally substituted with one or two —COOH;
each R^{1W} is independently H or C₁₋₃alkyl;
each R^{1X} is H or C₁₋₄alkyl; wherein the C₁₋₄alkyl is optionally substituted with one or two —COOH; and
each R^{1Y} is H or C₁₋₄alkyl; wherein the C₁₋₄alkyl is optionally substituted with one or two —COOH.

22.-27. (canceled)

28. The compound of claim 1, or the pharmaceutically acceptable salt thereof, wherein each R^{1F} is independently a —CH₃ optionally substituted with one or two substituents independently selected from the group consisting of —COOH, —P(O)(OH)₂, —OP(O)(OH)₂, —NR^{1G}R^{1H}, —CONR^{1G}R^{1H}, —OCOR^{1I}, and —NHCOR^{1I}.

29. (canceled)

30. The compound of claim 1, or the pharmaceutically acceptable salt thereof, wherein R^{1G} and R^{1H} are each independently H, —COO(CR^{1N}R^{1O})ₑOP(O)(OH)₂, or C₁₋₂alkyl, wherein the C₁₋₂alkyl is optionally substituted with one, two, or three substituents independently selected from a group consisting of halo, —COOR^{1M}, —OR^{1W}, —P(O)(OH)₂, and —CONR^{1J}R^{1K}.

31.-36. (canceled)

37. The compound of claim 1, or the pharmaceutically acceptable salt thereof, wherein X is pyridyl; wherein the pyridyl is optionally substituted with one, two or three substituents independently selected from the group consisting of —OP(O)(OH)₂, R^{1F}—COOH, and —CONR^{1X}R^{1Y};
each R^{1F} is independently a C₁₋₃alkyl optionally substituted with one or two substituents independently selected from the group consisting of —COOH, —P(O)(OH)₂, —OP(O)(OH)₂, —NR^{1G}R^{1H}, —CONR^{1G}R^{1H} and —OCOR^{1I};
each R^{1G} is independently H or —COO(CR^{1N}R^{1O})OP(O)(OH)₂;
each R^{1H} is independently CH₃ optionally substituted with one or two substituents independently selected from a group consisting of halo, —COOR^{1M}, —OR^{1W}, and —CONR^{1J}R^{1K};
each R^{1I} is independently C₁₋₆alkyl optionally substituted with one or two substituents selected independently from —COOH and —NR^{1J}R^{1K};
each R^{1J} is independently H or CH₃;
each R^{1K} is independently H or C₁₋₃alkyl, wherein the C₁₋₃alkyl is optionally substituted with one or two —COOH;
each R^{1M} is independently H, CH₃, tert-butyl, or benzyl;
each R^{1N} is independently H or CH₃;
each R^{1O} is independently H or CH₃;
each R^{1W} is independently H or CH₃;
e is 1 or 2;
each R^{1X} is H or C₁₋₄alkyl; wherein the C₁₋₄alkyl is optionally substituted with one or two —COOH; and
each R^{1Y} is H or C₁₋₄alkyl; wherein the C₁₋₄alkyl is optionally substituted with one or two —COOH.

38.-42. (canceled)

43. The compound of claim 1, or the pharmaceutically acceptable salt thereof, wherein X is 4 to 7 membered heterocyclyl comprising 1, 2 or 3 heteroatoms selected from N, O, and S; wherein the heterocyclyl is optionally substituted with one, two or three substituents independently selected from the group consisting of C₁₋₄alkyl, —COOC₁₋₄alkyl, oxo, and —COOCH₂OP(O)(OH)₂, wherein the C₁₋₄alkyl is optionally substituted with —OP(O)(OH)₂ or —NR^{1Z}R^{1AA};
wherein R^{1Z} is H, —COO(CR^{1AB}R^{1AC})ₕOP(O)(OH)₂ or C₁₋₄alkyl wherein the C14 alkyl is optionally substituted with one or two —COOH;
R^{1AA} is H, —COO(CR^{1AD}R^{1AE})ᵢOP(O)(OH)₂ or C₁₋₄alkyl wherein the C₁₋₄ alkyl is optionally substituted with one or two —COOH;
each R^{1AB} is independently H or C₁₋₃alkyl;
each R_{1AC} is independently H or C₁₋₃alkyl;
each R^{1AD} is independently H or C₁₋₃alkyl;
each R^{1AE} is independently H or C₁₋₃alkyl;
h is 1, 2, or 3; and
i is 1, 2, or 3.

44.-45. (canceled)

46. The compound of claim 1, or the pharmaceutically acceptable salt thereof, wherein X is 4 to 7 membered heterocyclyl comprising 1, 2 or 3 heteroatoms selected from N, O, and S; wherein the heterocyclyl is optionally substituted with one, two or three substituents independently selected from the group consisting of C₁₋₄alkyl, —COOC₁₋₄alkyl, oxo, and —COOCH₂OP(O)(OH)₂, wherein the C₁₋₄alkyl is optionally substituted with —OP(O)(OH)₂.

47.-59. (canceled)

60. The compound of claim 1, wherein X is C₃₋₇ cycloalkyl optionally substituted with one, two or three substituents independently selected from the group consisting of C₁₋₄alkyl, —COOC₁₋₄alkyl, oxo, —COOR^{1P}, and —OP(O)(OH)₂, wherein the C₁₋₄alkyl is optionally substituted with —OP(O)(OH)₂ or —NR^{1Q}R^{1R},
wherein R^{1P} is H or C₁₋₃ alkyl;
R^{1Q} is H, —COO(CR^{1S}R^{1T})_{f}OP(O)(OH)₂, or C₁₋₄alkyl; wherein the C₁₋₄alkyl is optionally substituted with —COOH;
R^{1R} is H, —COO(CR^{1U}R^{1V})_{g}OP(O)OH)₂, or C₁₋₄alkyl; wherein the C₁₋₄alkyl is optionally substituted with —COOH;
each R^{1S}, R^{1T}, R^{1U}, and R^{1V} is independently H or C₁₋₃alkyl;
f is 1, 2, or 3; and
g is 1, 2, or 3.

61.-63. (canceled)

64. The compound of claim 1, or the pharmaceutically acceptable salt thereof, wherein R¹ is selected from the group consisting of:

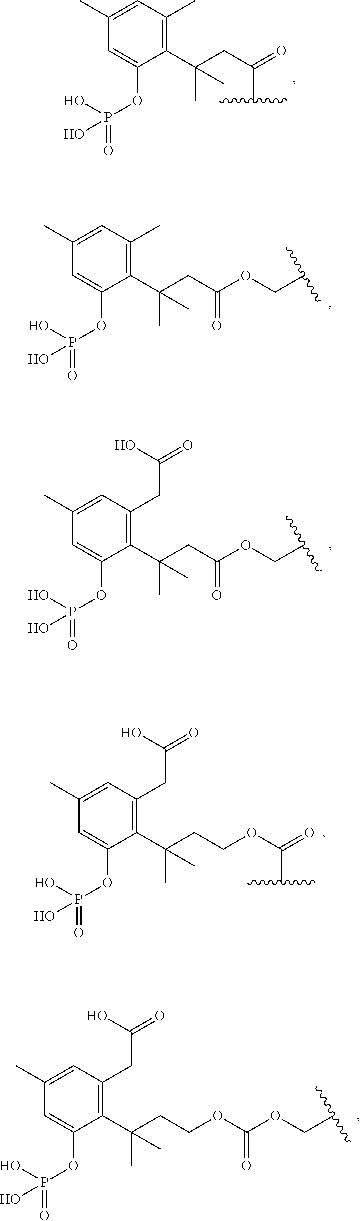 [SMILES: *C(=O)CC(C)(C)c1c(C)cc(C)cc1OP(=O)(O)O ; *COC(=O)CC(C)(C)c1c(C)cc(C)cc1OP(=O)(O)O ; *COC(=O)CC(C)(C)c1c(CC(=O)O)cc(C)cc1OP(=O)(O)O ; *C(=O)OCCC(C)(C)c1c(CC(=O)O)cc(C)cc1OP(=O)(O)O ; *COC(=O)OCCC(C)(C)c1c(CC(=O)O)cc(C)cc1OP(=O)(O)O]

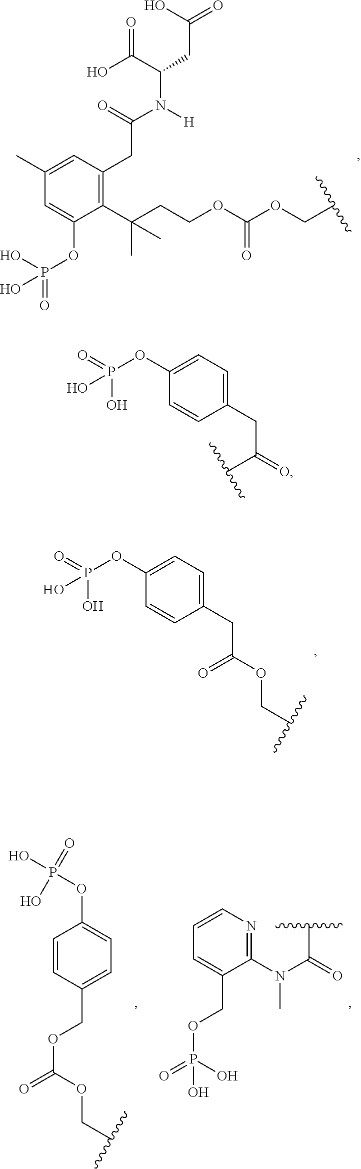 [SMILES: *COC(=O)OCCC(C)(C)c1c(CC(=O)N[C@@H](CC(=O)O)C(=O)O)cc(C)cc1OP(=O)(O)O ; *C(=O)Cc1ccc(OP(=O)(O)O)cc1 ; *COC(=O)Cc1ccc(OP(=O)(O)O)cc1 ; *COC(=O)OCc1ccc(OP(=O)(O)O)cc1 ; *C(=O)N(C)c1ncccc1COP(=O)(O)O]

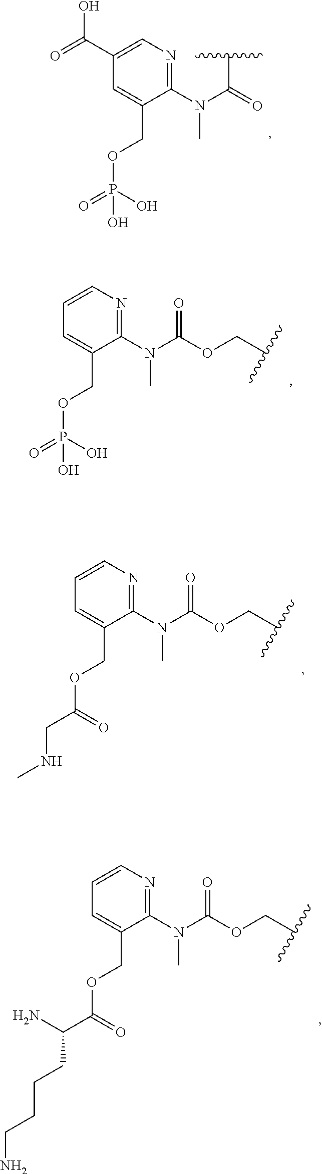 [SMILES: *C(=O)N(C)c1ncc(C(=O)O)cc1COP(=O)(O)O ; *COC(=O)N(C)c1ncccc1COP(=O)(O)O ; *COC(=O)N(C)c1ncccc1COC(=O)CNC ; *COC(=O)N(C)c1ncccc1COC(=O)[C@@H](N)CCCCN]

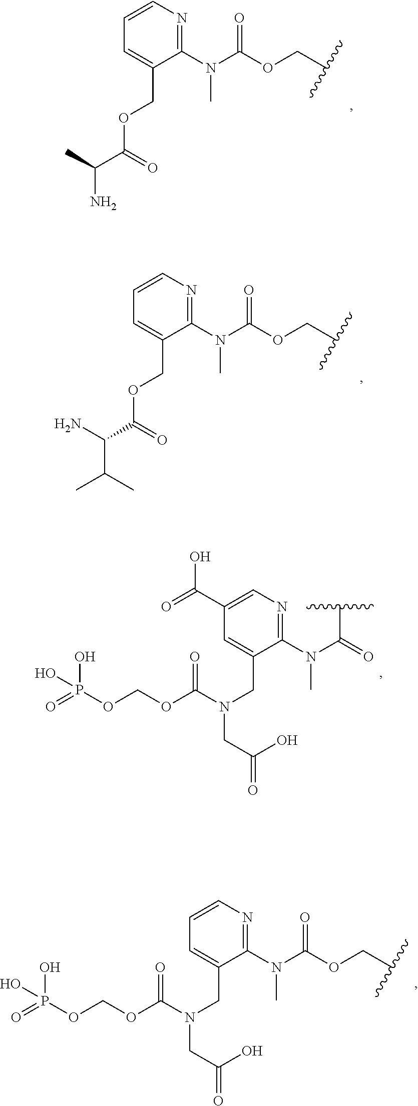 [SMILES: *COC(=O)N(C)c1ncccc1COC(=O)[C@H](C)N ; *COC(=O)N(C)c1ncccc1COC(=O)[C@@H](N)C(C)C ; *C(=O)N(C)c1ncc(C(=O)O)cc1CN(CC(=O)O)C(=O)OCOP(=O)(O)O ; *COC(=O)N(C)c1ncccc1CN(CC(=O)O)C(=O)OCOP(=O)(O)O]

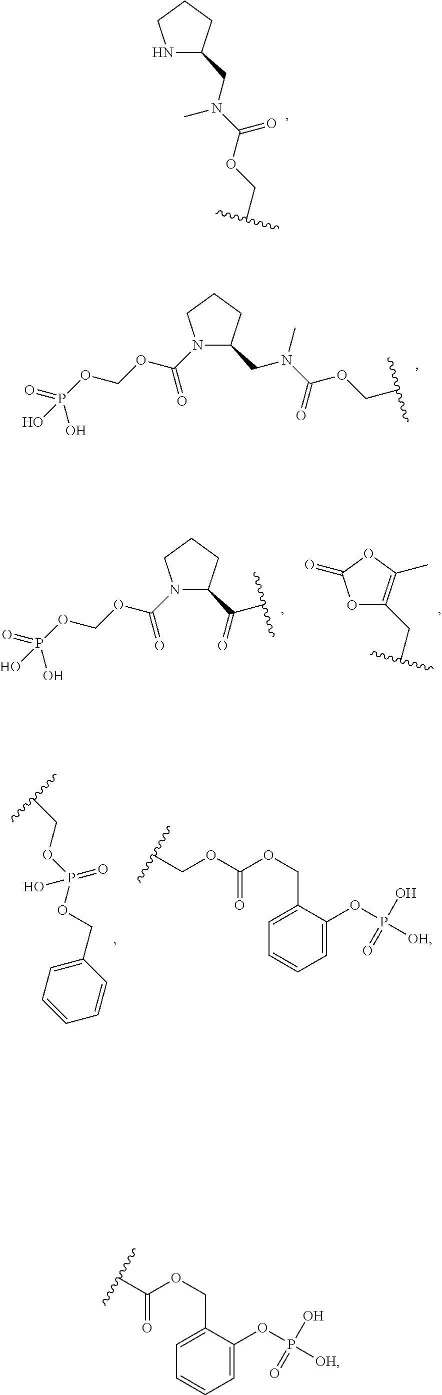

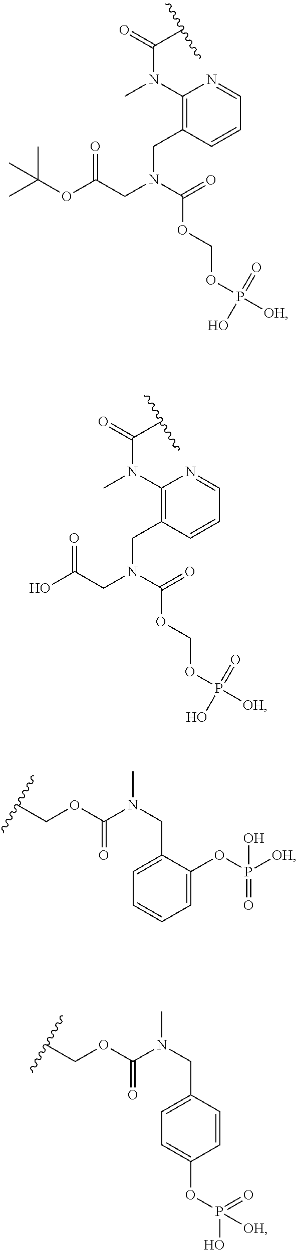 [SMILES: *C(=O)N(C)c1ncccc1CN(CC(=O)OC(C)(C)C)C(=O)OCOP(=O)(O)O ; *C(=O)N(C)c1ncccc1CN(CC(=O)O)C(=O)OCOP(=O)(O)O ; *COC(=O)N(C)Cc1ccccc1OP(=O)(O)O ; *COC(=O)N(C)Cc1ccc(OP(=O)(O)O)cc1]

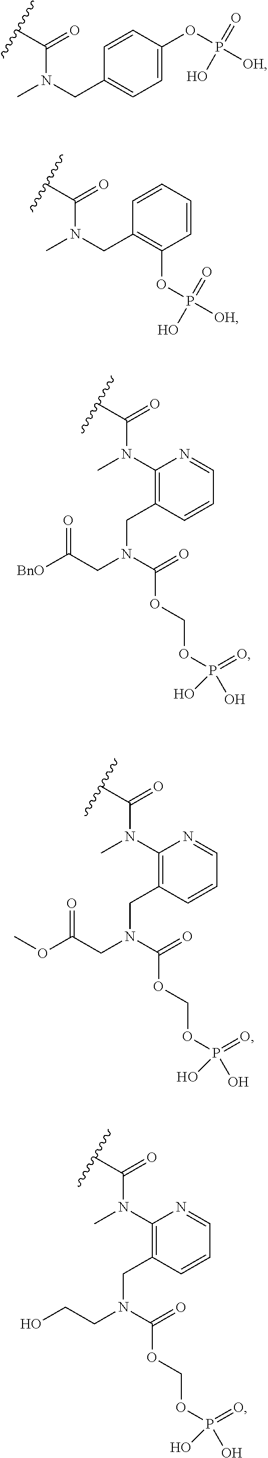 [SMILES: *C(=O)N(C)Cc1ccc(OP(=O)(O)O)cc1 ; *C(=O)N(C)Cc1ccccc1OP(=O)(O)O ; *C(=O)N(C)c1ncccc1CN(CC(=O)OCc1ccccc1)C(=O)OCOP(=O)(O)O ; *C(=O)N(C)c1ncccc1CN(CC(=O)OC)C(=O)OCOP(=O)(O)O ; *C(=O)N(C)c1ncccc1CN(CCO)C(=O)OCOP(=O)(O)O]

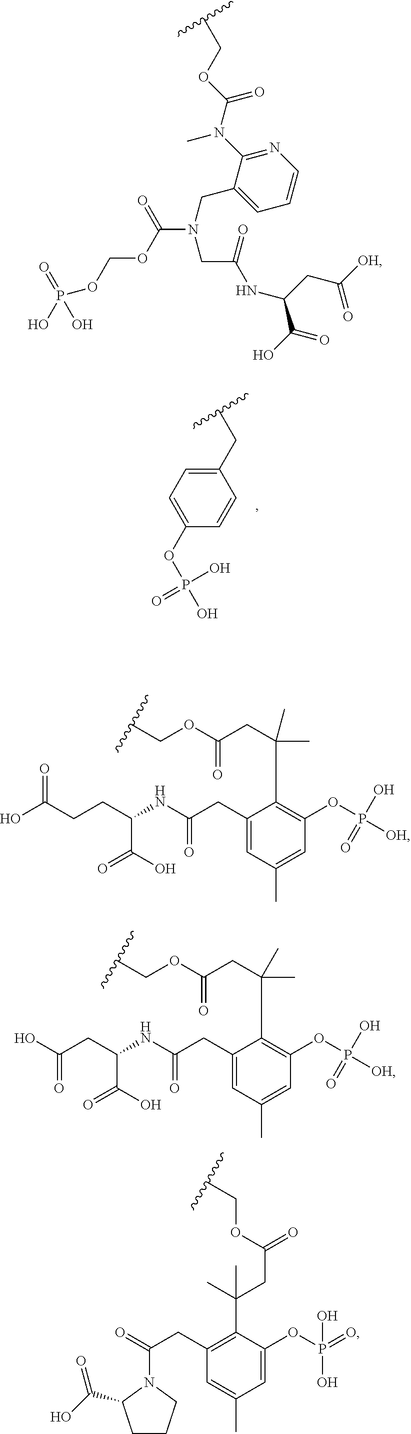

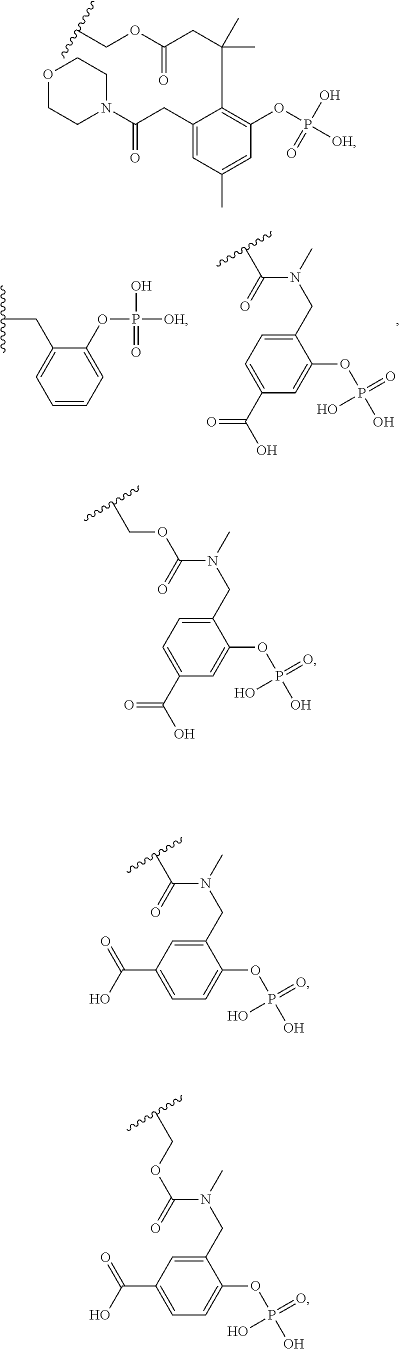 [SMILES: *COC(=O)CC(C)(C)c1c(CC(=O)N2CCOCC2)cc(C)cc1OP(=O)(O)O ; *Cc1ccccc1OP(=O)(O)O ; *C(=O)N(C)Cc1ccc(C(=O)O)cc1OP(=O)(O)O ; *COC(=O)N(C)Cc1ccc(C(=O)O)cc1OP(=O)(O)O ; *C(=O)N(C)Cc1cc(C(=O)O)ccc1OP(=O)(O)O ; *COC(=O)N(C)Cc1cc(C(=O)O)ccc1OP(=O)(O)O]

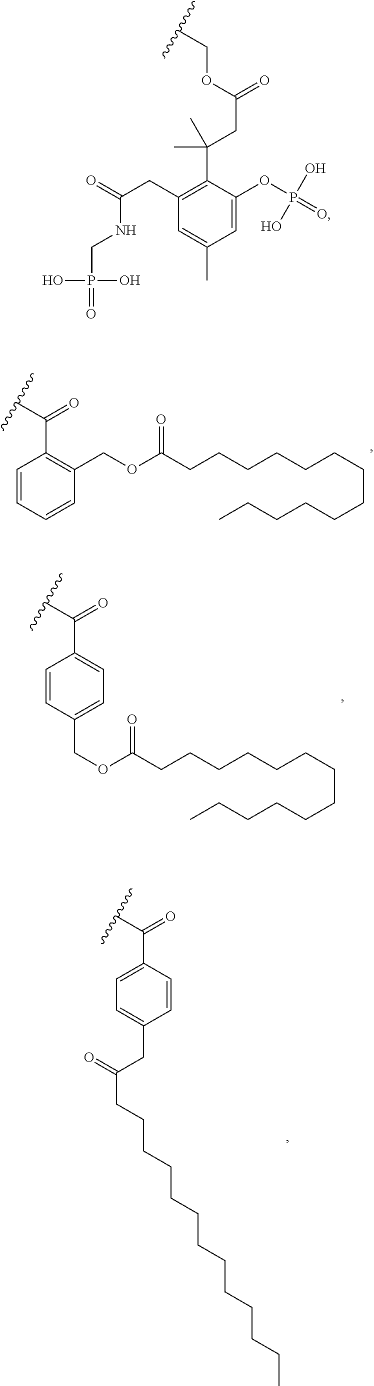 [SMILES: *COC(=O)CC(C)(C)c1c(CC(=O)NCP(=O)(O)O)cc(C)cc1OP(=O)(O)O ; *C(=O)c1ccccc1COC(=O)CCCCCCCCCCCCC ; *C(=O)c1ccc(COC(=O)CCCCCCCCCCCCC)cc1 ; *C(=O)c1ccc(CC(=O)CCCCCCCCCCCCC)cc1]

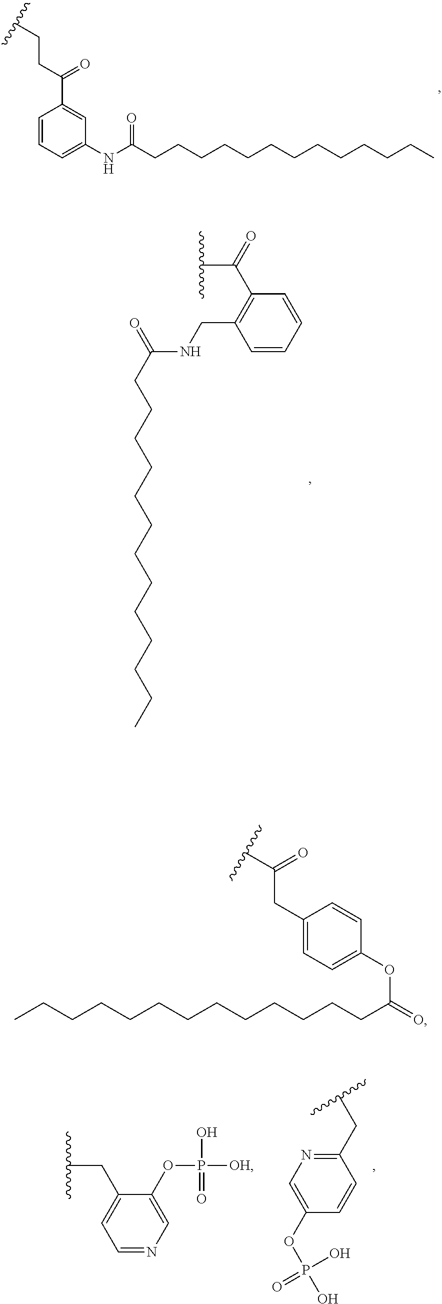

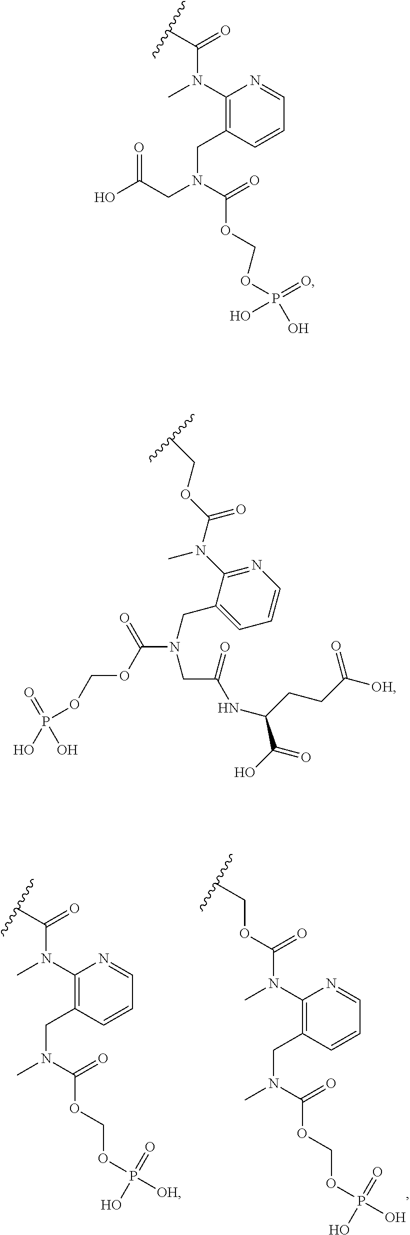 [SMILES: *C(=O)N(C)c1ncccc1CN(CC(=O)O)C(=O)OCOP(=O)(O)O ; *COC(=O)N(C)c1ncccc1CN(CC(=O)N[C@@H](CCC(=O)O)C(=O)O)C(=O)OCOP(=O)(O)O ; *C(=O)N(C)c1ncccc1CN(C)C(=O)OCOP(=O)(O)O ; *COC(=O)N(C)c1ncccc1CN(C)C(=O)OCOP(=O)(O)O]

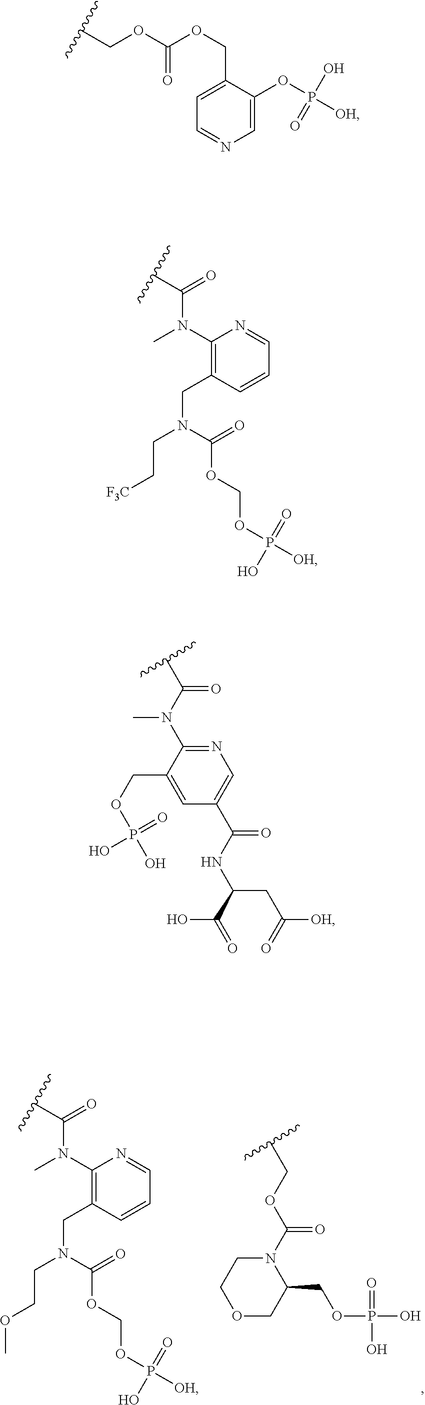 [SMILES: *COC(=O)OCc1ccncc1OP(=O)(O)O ; *C(=O)N(C)c1ncccc1CN(CCC(F)(F)F)C(=O)OCOP(=O)(O)O ; *C(=O)N(C)c1ncc(C(=O)N[C@@H](CC(=O)O)C(=O)O)cc1COP(=O)(O)O ; *C(=O)N(C)c1ncccc1CN(CCOC)C(=O)OCOP(=O)(O)O ; *COC(=O)N1CCOC[C@@H]1COP(=O)(O)O]

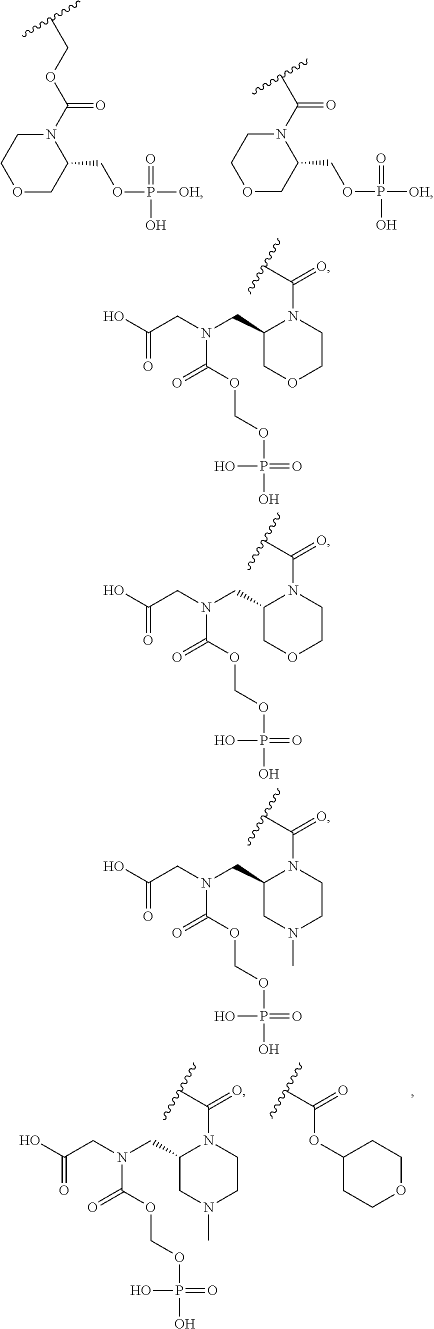

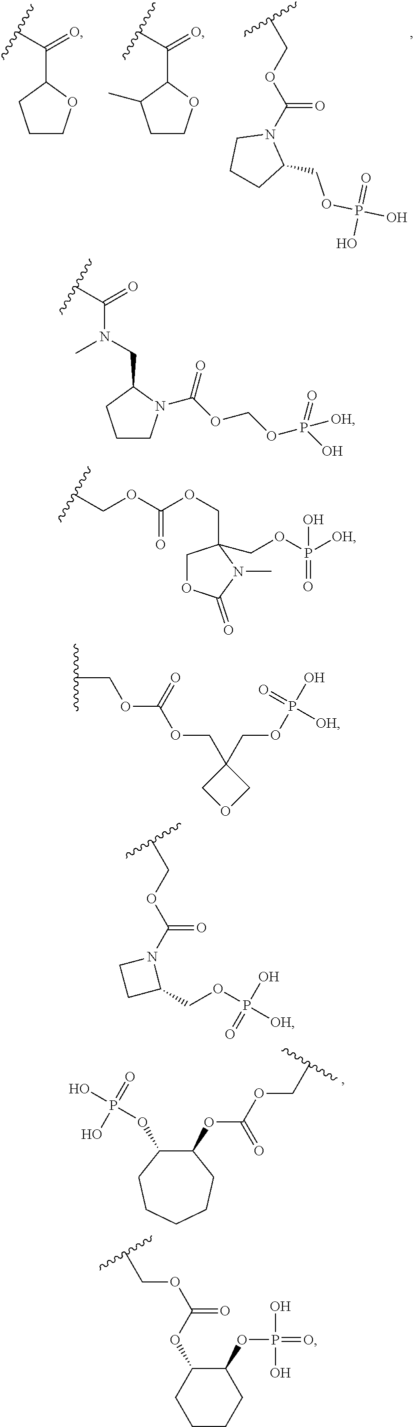

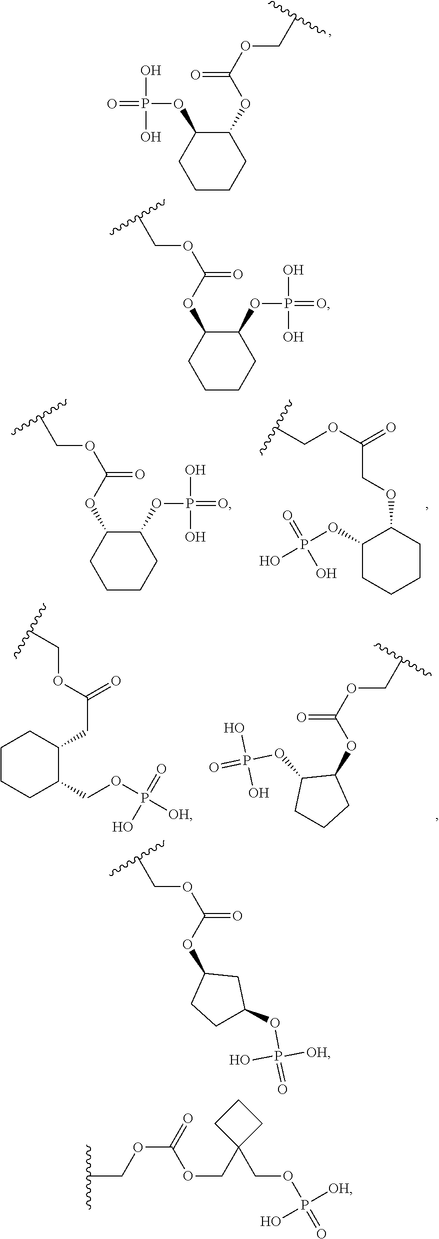

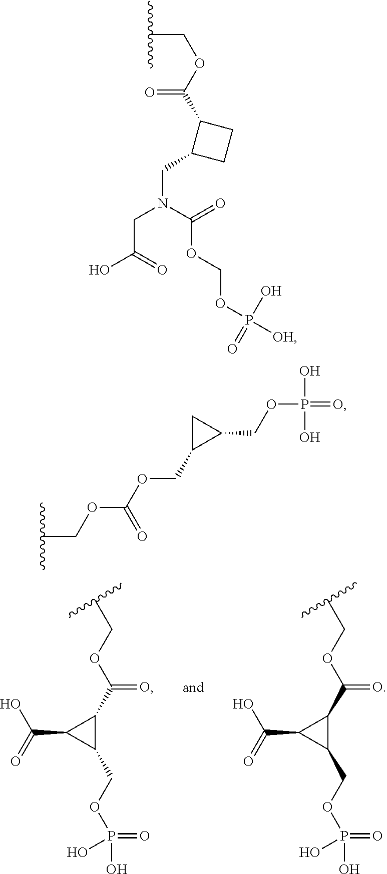

65.-72. (canceled)

73. A pharmaceutical composition comprising a therapeutically effective amount of a compound of claim 1, or pharmaceutically acceptable salt thereof, and a pharmaceutically acceptable excipient.

74.-83. (canceled)

84. A method of treating an HIV infection in a human having or at risk of having the infection, comprising administering to the human a therapeutically effective amount of a compound of claim 1, or a pharmaceutically acceptable salt thereof.

85.-93. (canceled)
